(19) United States
(12) Patent Application Publication
(10) Pub. No.: US 2024/0287487 A1
(43) Pub. Date: Aug. 29, 2024

(54) IMPROVED CYTOSINE TO GUANINE BASE EDITORS

(71) Applicants: The Broad Institute, Inc., Cambridge, MA (US); President and Fellows of Harvard College, Cambridge, MA (US); The Regents of the University of California, Oakland, CA (US); Massachusetts Institute of Technology, Cambridge, MA (US)

(72) Inventors: Luke W. Koblan, Cambridge, MA (US); David R. Liu, Cambridge, MA (US); Mandana Arbab, Cambridge, MA (US); Max Walt Shen, Cambridge, MA (US); Andrew Vito Anzalone, Cambridge, MA (US); Jeffrey Hussmann, Oakland, CA (US)

(73) Assignees: The Broad Institute, Inc., Cambridge, MA (US); President and Fellows of Harvard College, Cambridge, MA (US); The Regents of the University of California, Oakland, CA (US); Massachusetts Institute of Technology, Cambridge, MA (US)

(21) Appl. No.: 18/568,796

(22) PCT Filed: Jun. 10, 2022
(86) PCT No.: PCT/US2022/033121

§ 371 Date: Dec. 8, 2023

Related U.S. Application Data
(60) Provisional application No. 63/209,881, filed on Jun. 11, 2021.

(51) Int. Cl. C12N 9/22; A61K 38/00; C07K 14/35; C12N 9/10; C12N 9/12; C12N 9/78; C12N 15/11; C12N 15/90

(52) U.S. Cl. CPC C12N 9/22; C07K 14/35; C12N 9/104; C12N 9/1252; C12N 9/78; C12N 15/11; C12N 15/907; C12Y 207/07007; C12Y 305/04005; A61K 38/00; C07K 2319/00; C12N 2310/20; C12N 2800/80

(57) ABSTRACT

Aspects of this disclosure provide compositions, strategies, systems, reagents, methods, and kits that are useful for the targeted editing of nucleic acids, including editing a single site within the genome of a cell or subject, e.g., within the human genome. Fusion proteins capable of inducing a cytosine (C) to guanine (G) change (i.e., transversion changes) in a nucleic acid (e.g., genomic DNA) are provided. Fusion proteins of a nucleic acid programmable DNA binding protein (e.g., Cas9) and nucleic acid editing proteins or protein domains, e.g., deaminase domains, polymerase domains, base excision enzymes, and/or DNA repair proteins, are also provided. Methods for targeted nucleic acid editing are also provided. Reagents and kits for the generation of targeted nucleic acid editing proteins, e.g., fusion proteins of a nucleic acid programmable DNA binding protein (e.g., Cas9), and nucleic acid editing proteins or domains, are further provided in the present disclosure.

### RELATED APPLICATIONS

This application claims priority under 35 U.S.C. § 119(e) to U.S. Provisional Application, U.S. Ser. No. 63/209,881, filed Jun. 11, 2021, which is incorporated herein by reference.

### BACKGROUND OF INVENTION

Targeted editing of nucleic acid sequences, for example, the targeted cleavage or the targeted introduction of a specific modification into genomic DNA, is a highly promising approach for the study of gene function and also has the potential to provide new therapies for human genetic diseases. Since many genetic diseases in principle can be treated by effecting a specific nucleotide change at a specific location in the genome (for example, a C to G or a G to C change in a specific codon of a gene associated with a disease), the development of a programmable way to achieve such precise gene editing represents both a powerful new research tool, as well as a potential new approach to gene editing-based therapeutics.

Two primary classes of base editors have been generally described to date: cytosine base editors convert target C:G base pairs to T:A base pairs, and adenosine base editors convert A:T base pairs to G:C base pairs. Collectively, these two classes of base editors enable the targeted installation of all possible transition mutations (C-to-T, G-to-A, A-to-G, T-to-C, C-to-U, and A-to-U), which collectively account for about 61% of known human pathogenic single nucleotide polymorphisms (SNPs) in the ClinVar database. See Gaudelli, N. M. et al., Programmable base editing of A:T to G:C in genomic DNA without DNA cleavage. Nature 551, 464-471 (2017), which is incorporated herein by reference.

For instance, C-to-T base editors use a cytidine deaminase to convert cytidine to uracil in the single-stranded DNA loop created by the Cas9 (“CRISPR-associated protein 9”) domain. The opposite strand is nicked by Cas9 to stimulate DNA repair mechanisms that use the edited strand as a template, while a fused uracil glycosylase inhibitor slows excision of the edited base. Eventually, DNA repair leads to a C:G to T:A base pair conversion. This class of base editor is described in U.S. Patent Publication No. 2017/0121693, published May 4, 2017, which issued on Jan. 1, 2019, as U.S. Pat. No. 10,167,457, which is incorporated herein by reference. Cytosine and adenosine base editors are not capable, however, of generating transversion mutations. Accordingly, there is a need for transversion base editors.

### SUMMARY OF THE INVENTION

A major limitation of base editing is the inability to generate transversion (purine↔pyrimidine) changes, which are needed to correct the remaining ˜38% of known human pathogenic SNPs. See Komor, A. C. et al., Programmable editing of a target base in genomic DNA without double-stranded DNA cleavage, Nature 533, 420-424 (2016); and Landrum, M. J. et al., ClinVar: public archive of relationships among sequence variation and human phenotype, Nucleic Acids Res. 42, D980-985 (2014), each of which is incorporated herein by reference. Traditionally, transversions could only be repaired by nuclease-mediated formation of a double-stranded break (DSB) followed by homology directed repair (HDR), which is typically inefficient, especially in non-mitotic cells, and leads to undesired byproducts such as indels (insertions and deletions) and translocations. See Komor, A. C., Badran, A. H. & Liu, D. R. CRISPR-Based Technologies for the Manipulation of Eukaryotic Genomes, Cell 168, 20-36, (2017), herein incorporated by reference. Since nucleobase deamination alone cannot interconvert purines and pyrimidines, the development of transversion base editors has required the incorporation of novel editing strategies, such as the manipulation of endogenous DNA repair pathways or a different nucleobase chemical transformation. See for instance, International Publication Nos. WO 2018/165629, which published on Sep. 13, 2018, WO 2020/102659, which published on May 22, 2020, WO 2020/181178, which published on Sep. 10, 2020, WO 2020/181180, which published on Sep. 10, 2020, WO 2020/181195, which published on Sep. 10, 2020, and WO 2021/030666, which published on Feb. 18, 2021, each of which are incorporated herein in their entireties.

The disclosure provides CGBEs that exhibit higher editing yields, higher product purities, and/or lower bystander editing efficiencies than previously described CGBEs, such as those described in International Publication No. WO 2018/165629, published Sep. 13, 2018; Kurt, I. C. et al. Nature Biotechnology 39, 41-46 (2020); Zhao, D. et al. Nature Biotechnology 39, 35-40 (2020); and Chen, L. et al., Nature Communications 12 (2021), each of which is incorporated by reference herein. The presently disclosed CGBEs may contain multiple uracil binding protein (UBP) domains, whereas the previously described CGBEs contain a single uracil binding protein domain. Use of multiple UBPs, and in particular UBPs that bind tightly to uracil with minimal uracil excising activity, may increase the occurrence of C to G editing following formation of an abasic site.

In other aspects, the disclosed CGBEs may contain one or more domains containing a protein implicated in DNA repair (referred to herein as “DNA repair protein domains”) that are not present in previously described CGBEs. In other aspects, the disclosed CGBEs may contain a nucleic acid programmable DNA binding protein (napDNAbp) domain containing a Cas9 variant different from the Cas9 protein domains used in previously described CGBEs, including recently generated Cas9 variants that have expanded targeting scope or higher DNA base specificities. In some embodiments, the disclosed CGBEs contain a DNA repair protein domain and a napDNAbp domain containing a Cas9 variant. In some embodiments, these CGBEs contain a single UBP domain. In some embodiments, these CGBEs contain two or more UBP domains, such as a first UBP domain and a second UBP domain.

The disclosed CGBEs may exhibit broader sequence substrate scope, thus enabling efficient editing at a greater number of genomic loci, than previously described CGBEs. At several genomic loci, the disclosed CGBEs may outperform previously described CGBEs.

Accordingly, provided herein are improved base editors, vectors encoding these base editors, complexes of these base editors and a guide RNA, cells and compositions comprising these base editors, and methods of modifying a polynucleotide (e.g., DNA) for generating a cytosine to guanine substitution in the polynucleotide. As described in greater detail herein, base editing (e.g., C to G editing) is accomplished by deaminating a cytosine (C) nucleobase leading to excision of the resulting uracil, thereby generating an abasic site within a nucleic acid sequence. The nucleobase opposite the abasic site (e.g., guanine), is then replaced with a different nucleobase (e.g., cytosine), for example, by an endogenous translesion polymerase. Base editing fusion proteins described herein are capable of generating specific mutations (C to G mutations), within a nucleic acid (e.g., genomic DNA), which can be used, for example, to treat diseases involving nucleic acid mutations, e.g., C to G, or G to C mutations.

As disclosed in International Publication No. WO 2018/165629, published Sep. 13, 2018, which is incorporated herein by reference, an example of a C to G base editor includes a fusion protein containing a nucleic acid programmable DNA binding protein domain (e.g., a Cas9 domain), a uracil binding protein (UBP) domain, and a cytidine deaminase domain. This publication disclosed fusion proteins containing a single uracil binding protein domain, such as a single UdgX domain, an orthologue of Uracil N-glycosylase (UNG) identified to bind tightly to uracil. The UdgX domain has been shown to increase the amount of C to G editing. Without wishing to be bound by any particular theory, such base editing fusion proteins are capable of binding to a specific nucleic acid sequence (e.g., via the Cas9 domain), deaminating a cytosine within the nucleic acid sequence to a uracil, which is then excised from the nucleic acid molecule by the UDG domain. The nucleobase opposite the abasic site can then be replaced with another base (e.g., cytosine), for example, by an endogenous translesion polymerase. More often than 25% of the time, the cell's base repair machinery replaces a nucleobase opposite an abasic site with a cytosine.

Cytosine-to-guanine base editing fusion proteins include a nucleic acid programmable DNA binding protein (e.g., a Cas9 domain), and a base excision enzyme that removes a nucleobase (e.g., a cytosine). Rather than deaminating a cytosine to uracil and excising the uracil using a UDG, as described above, a base editor may include a base excision enzyme that recognizes and removes a nucleobase such as a cytosine or a thymine without first deaminating it. Accordingly, base editors (e.g., C to G base editors) have been engineered by fusing a nucleic acid programmable DNA binding protein (e.g., a Cas9 domain) to a base excision enzyme that removes cytosine or thymine from a nucleic acid molecule. Furthermore, as with the base editor described above, translesion polymerases may be incorporated into this base editor to increase the cytosine incorporation opposite an abasic site generated by the base excision enzyme of the base editor. Exemplary base editing proteins and schematic representations outlining cytosine-to-guanine base editing strategies can be seen, for example, in FIGS. 1-6, 33-36, 40, 48, and 52.

The improved CGBEs provided herein make use of fusion proteins that include additional domains not included in previously disclosed CGBEs. These domains may include multiple uracil binding proteins, such as multiple uracil DNA glycosylase proteins (e.g., multiple UdgX protein domains), proteins implicated in DNA repair, and/or Cas9 variants not included in previously disclosed CGBEs, including Cas9 variants having higher DNA base specificities.

Accordingly, in some embodiments, the disclosure provides fusion proteins that are capable of cytosine to guanine base editing. The presently disclosed CGBEs contain one or more UBP domains. In various embodiments, the UBP domain is a a UNG orthologue from Mycobacterium smegmatis (or B. smegmatis or M. smegmatis) (UdgX) protein. The inventors have demonstrated that efficient CGBE editing is achieved when, for instance, the fusion protein contains an architecture comprising NH₂-[cytidine deaminase domain]-[first UBP domain]-[napDNAbp domain]-COOH, wherein each instance of “]-[” comprises an optional linker. For instance, efficient CGBE editing is achieved when the fusion protein contains a structure that comprises NH₂-[APOBEC1 deaminase domain]-[UdgX domain]-[Cas9 domain]-COOH, which is an architecture referred to herein as the “AXC” architecture.

Thus, in some aspects, a CGBE fusion protein may comprise (i) a napDNAbp domain, (ii) a cytidine deaminase domain, (iii) a first UBP domain, and (iv) a second UBP domain. These fusion proteins may further comprise a third UBP domain. In various embodiments, at least one of the first, second, and third UBP domains is a a UNG orthologue from Mycobacterium smegmatis (UdgX) protein. In some embodiments, each of the first and second, and/or third, UBP domain is a UdgX protein.

The disclosure is based, at least in part, on a focused CRISPR interference (CRISPRi) screen to identify DNA repair genes that impact cytosine base editing efficiency and purity. Guided by these data, various fusions proteins were constructed containing deaminases and Cas proteins fused to DNA repair proteins to generate novel CGBEs. These DNA repair proteins include DNA polymerase D2 (POLD2), exonuclease 1 (EXO1), and RNA binding motif protein X-linked (RBMX). In some aspects, the improved CGBEs contain a DNA repair protein domain. Accordingly, in some aspects, the fusion protein includes (i) a napDNAbp domain, (ii) a cytidine deaminase domain, (iii) a first UBP domain, and (iv) a DNA repair protein. Without being bound to a particular theory, the protein of this domain may be implicated in DNA repair in the traditional sense. In other embodiments, the protein of this domain is implicated in DNA repair by virtue of the results of a CRISPRi screen to identify DNA repair genes that impact cytosine base editing efficiency and purity.

Accordingly, in some embodiments, the DNA repair protein is selected from a DNA polymerase, an exonuclease, an RNA binding motif protein, an E3 ligase, and a translesion polymerase. In particular embodiments, the DNA repair protein is one of POLD2, RBMX, and EXO1. In some embodiments, the DNA repair protein is a nucleic acid polymerase, such as a DNA polymerase (e.g., a translesion polymerase). In various embodiments, the DNA repair protein is selected from DNA polymerase D1 (POLD1), DNA polymerase D2 (POLD2), and DNA polymerase D3 (POLD3).

In some aspects, the CGBEs of the disclosure include a napDNAbp domain that is a Cas9 variant having a higher targeting specificity than the napDNAbp domains of previously disclosed CGBEs. In some embodiments, the napDNAbp domain is selected from a HypaCas9, an HF-nCas9-NG, a Sniper-Cas9, a Hypa-nCas9, an HF-Hypa-nCas9, an e-Cas9, an e-HF-Hypa-nCas9, and an e-Hypa-Cas9, or the napDNAbp is at least 80%, at least 85%, at least 90%, at least 95%, at least 96%, at least 97%, at least 98%, or at least 99% identical to the amino acid sequence of any one of HypaCas9, an HF-nCas9-NG, a Sniper-Cas9, a Hypa-nCas9, an HF-Hypa-nCas9, an e-Cas9, an e-HF-Hypa-nCas9, and an e-Hypa-Cas9. In some aspects, the napDNAbp domain is selected from an HF-nCas9-NG, an HF-Hypa-nCas9, and an e-HF-Hypa-nCas9. In some embodiments, the CGBEs of the disclosure may comprise: (i) a napDNAbp domain, (ii) a cytidine deaminase domain, (iii) a first uracil binding protein (UBP) domain, and (iv) a DNA repair protein; or (i) a napDNAbp domain, (ii) a cytidine deaminase domain, (iii) a first UBP domain, and (iv) a second UBP domain, wherein the napDNAbp domain is selected from a HypaCas9, a HF-nCas9-NG, a Sniper-Cas9, an HF-Hypa-nCas9, an e-Cas9, an e-HF-Hypa-nCas9, and an e-Hypa-Cas9. In some embodiments, the napDNAbp domain of any of the disclosed CGBEs comprises an amino acid sequence that is at least 85%, 90%, 92.5%, 95%, 97%, 98%, or 99% identical to any of the sequences set forth as SEQ ID NOs: 726-736. In some embodiments, the napDNAbp domain of any of the disclosed CGBEs is selected from SEQ ID NOs: 726-736.

In other aspects, it was found that incorporating into the base editor a nucleic acid polymerase (NAP) domain, such as a translesion polymerase, in place of or in addition to the DNA repair protein domain, can increase the percentage of cytosine incorporation opposite an abasic site. Accordingly, base editors were engineered to incorporate various translesion polymerase domains to improve base editing efficiency. Translesion polymerases that increase the preference for C integration opposite an abasic site can improve the efficiency of C to G nucleobase editing.

The present disclosure further provides complexes comprising the cytosine-to-guanine base editors described herein and a guide RNA associated with the napDNAbp domain of the base editor, such as a single guide RNA. The guide RNA may be 15-100 nucleotides in length, and/or the guide RNA comprise a sequence of at least 10, at least 15, or at least 20 contiguous nucleotides that is complementary to a target nucleotide sequence.

The present disclosure further provides methods of DNA editing that make use of the base editors disclosed herein. These methods may induce (or yield, provide, or cause) an actual or average efficiency of conversion of C to G of at least about 70%, 73%, 75%, 77%, 80%, 82%, 83%, 84%, 86%, 88%, 90%, 92.5%, 95%, or 98% when contacted with a DNA molecule comprising a target sequence.

In other aspects, the disclosure provides polynucleotides and vectors encoding any of the base editors described herein. In some embodiments, the polynucleotides and vectors encode a gRNA. The nucleic acid sequences may be codon-optimized for expression in the cells of any organism of interest (e.g., a human).

In other aspects, the disclosure provides kits for expressing and/or transducing host cells with an expression construct encoding the base editor and gRNA. It further provides kits for administration of expressed base editors and expressed gRNA molecules to a host cell (such as a mammalian cell, e.g., a human cell). The disclosure further provides cells stably or transiently expressing the base editor and gRNA, or a complex thereof.

It should be appreciated that any of the base editors described herein may be introduced into the cell in any suitable way, either stably or transiently. In some embodiments, a base editor may be transfected into the cell. In some embodiments, the cell may be transduced or transfected with a nucleic acid construct that encodes a base editor. For example, a cell may be transduced (e.g., with a viral particle containing a vector encoding a base editor) with a nucleic acid that encodes a base editor, or the translated base editor. As an additional example, a cell may be transfected (e.g., with a plasmid encoding a base editor) with a nucleic acid that encodes a base editor or the translated base editor.

In some embodiments, methods of treatment using the base editors described herein are provided. The methods described herein may comprise treating a subject having or at risk of developing a disease, disorder, or condition associated with a G:C to C:G point mutation comprising administering to the subject an base editor as described herein, a polynucleotide as described herein, a vector as described herein, or a pharmaceutical composition as described herein. In some embodiments, methods of treatment of Ehlers-Danlos syndrome, Sotos syndrome, Cornelia de Lange syndrome, or a cancer using the base editors described herein are provided. In some embodiments, the present disclosure provides uses of any of the fusion proteins, complexes, vectors, cells, and pharmaceutical compositions provided herein as a medicament.

Base editors and methods of using base editors are described below in further detail.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

FIG. 1 shows a general schematic illustrating C to T and C to G base editing. Certain DNA polymerases (e.g., translesion polymerases) are known to replace bases opposite abasic sites with G. One strategy to achieve C to G base editing is to induce the creation of an abasic site, then recruit or tether such a polymerase to replace the G opposite the abasic site with a C.

FIG. 2 shows a general schematic illustrating base editing via abasic site generation and base-specific repair for C to G editing.

FIG. 3 shows a schematic illustrating Scheme 1 from FIG. 1, where an abasic site is formed, for C to G base editing. If the abasic is generated efficiently, this can increase the total flux through the C to G editing pathway.

FIG. 4 shows a schematic illustrating approach 1 for C to G base editing where an increase in abasic site formation is used. If the abasic is generated efficiently, for example, by using a UDG domain and a translesion polymerase, this can increase the total flux through the C to G editing pathway.

FIG. 5 shows a schematic illustrating the effect of UdgX on base editing. UdgX, an orthologue of UDG. In 1) UdgX* is a variant of UDG which was determined to lack uracil binding activity via an in vitro assay. In 2) UdgX_On is a variant which was shown to increase uracil excision through an in vitro assay. In 3) UDG direct fusion excises uracil.

FIG. 6 shows a schematic (on the left) illustrating an exemplary C to T base editor (e.g., BE3), which contains a uracil glycosylase inhibitor (UGI), a Cas9 domain (e.g., nCas9), and a cytidine deaminase. On the right is a schematic illustrating a C to G base editor, which contains a uracil DNA glycosylase (UDG) (or variants thereof), a Cas9 domain (e.g., nCas9), and a cytidine deaminase.

FIG. 7 shows total editing percentages at the HEK2 site in WT Hap1 cells using seven base editors (BE3; BE3_UdgX; BE3_UdgX*; BE2_UdgX_On; BE3_UdgX_On; BE2_UDG; and BE3_UDG). Raw editing values are shown in the left panel. The panel on the right shows a graphical representation of the raw editing values, where C to G base editing is graphically shown by dotted bars (G) going to filled bars (C), as sequencing was performed on the DNA strand opposite of the strand containing the edited C.

FIG. 8 shows total editing percentages at the HEK2 site with additional C to G base editors (BE3; BE3_UdgX; BE3_REV7; and SMUG1, where BE3 and BE3_UdgX are repeated from FIG. 4) in WT Hap1 cells. The top panel shows the raw editing values. The bottom panel shows a graphical representation of the raw editing values, where C to G base editing is graphically shown by dotted bars (G) going to filled bars (C), as sequencing was performed on the DNA strand opposite of the strand containing the edited C.

FIG. 9 shows the editing specificity ratio at the HEK2 site with various C to G base editors (BE3; BE3_UdgX; BE3_UdgX*; BE3_REV7; BE2_UDG; BE3_UDG BE2_UdgX_On; BE3_UdgX_On; and SMUG1) in WT Hap1 cells. The top panel shows the total percentage of edits and the ratio of edits that have been made from G to A, C, or T. The bottom panel is a graphical representation of the specificity ratio values.

FIG. 10 shows total editing percentages at the RNF2 site in WT Hap1 cells using seven base editors (BE3; BE3_UdgX; BE3_UdgX*; BE2_UdgX_On; BE3_UdgX_On; BE2_UDG; and BE3_UDG). Raw editing values are shown in the left panel. The panel on the right shows a graphical representation of the raw editing values, where C to G base editing is graphically shown by dotted bars (G) going to filled bars (C), as sequencing was performed on the DNA strand opposite of the strand containing the edited C.

FIG. 11 shows total editing percentages at the RNF2 site with additional C to G base editors (BE3; BE3_UdgX; BE3_REV7; and SMUG1, where BE3 and BE3_UdgX are repeated from FIG. 7) in WT Hap1 cells. The top panel shows the raw editing values. The bottom panel shows a graphical representation of the raw editing values, where C to G base editing is graphically shown by dotted bars (G) going to filled bars (C), as sequencing was performed on the DNA strand opposite of the strand containing the edited C.

FIG. 12 shows editing specificity ratio at the RNF2 site with various C to G base editors (BE3; BE3_UdgX; BE3_UdgX*; BE3_REV7; BE2_UDG; BE3_UDG BE2_UdgX_On; BE3_UdgX_On; and SMUG1) in WT Hap1 cells. The top panel shows the total percentage of edits and the ratio of edits that have been made from G to A, C, or T. The bottom panel is a graphical representation of the specificity ratio values.

FIG. 13 shows total editing percentages at the FANCF site in WT Hap1 cells using seven base editors (BE3; BE3_UdgX; BE3_UdgX*; BE2_UdgX_On; BE3_UdgX_On; BE2_UDG; and BE3_UDG). Raw editing values are shown in the left panel. The panel on the right shows a graphical representation of the raw editing values, where C to G base editing is graphically shown by filled bars (C) going to dotted bars (G).

FIG. 14 shows total editing percentages at the FANCF site with additional C to G base editors (BE3; BE3_UdgX; BE3_REV7; and SMUG1, where BE3 and BE3_UdgX are repeated from FIG. 10) in WT Hap1 cells. The top panel shows the raw editing values. The bottom panel shows a graphical representation of the raw editing values, where C to G base editing is graphically shown by filled bars (C) going to dotted bars (G).

FIG. 15 shows the editing specificity ratio at the FANCF site with various C to G base editors (BE3; BE3_UdgX; BE3_UdgX*; BE3_REV7; BE2_UDG; BE3_UDG BE2_UdgX_On; BE3_UdgX_On; and SMUG1) in WT Hap1 cells. The top panel shows the total percentage of edits and the ratio of edits that have been made from C to A, G, or T. The bottom panel is a graphical representation of the specificity ratio values.

FIG. 16 shows total editing percentages at the HEK2 site in UDG^{-/-} Hap1 cells using seven base editors (BE3; BE3_UdgX; BE3_UdgX*; BE2_UdgX_On; BE3_UdgX_On; BE2_UDG; and BE3_UDG). Raw editing values are shown in the left panel. The panel on the right shows a graphical representation of the raw editing values, where C to G base editing is graphically shown by dotted bars (G) going to filled bars (C), as sequencing was performed on the DNA strand opposite of the strand containing the edited C.

FIG. 17 shows total editing percentages at the HEK2 site with additional C to G base editors (BE3; BE3_UdgX; BE3_REV7; and SMUG1, where BE3 and BE3_UdgX are repeated from FIG. 13) in UDG^{-/-} Hap1 cells. The top panel shows the raw editing values. The bottom panel shows a graphical representation of the raw editing values, where C to G base editing is graphically shown by dotted bars (G) going to filled bars (C), as sequencing was performed on the DNA strand opposite of the strand containing the edited C.

FIG. 18 shows editing specificity ratio at the HEK2 site with various C to G base editors (BE3; BE3_UdgX; BE3_UdgX*; BE3_REV7; BE2_UDG; BE3_UDG BE2_UdgX_On; BE3_UdgX_On; and SMUG1) in UDG^{-/-} Hap1 cells. The top panel shows the total percentage of edits and the ratio of edits that have been made from G to A, C, or T. The bottom panel is a graphical representation of the specificity ratio values.

FIG. 19 shows total editing percentages at the RNF2 site in UDG^{-/-} Hap1 cells using seven base editors (BE3; BE3_UdgX; BE3_UdgX*; BE2_UdgX_On; BE3_UdgX_On; BE2_UDG; and BE3_UDG). Raw editing values are shown in the left panel. The panel on the right shows a graphical representation of the raw editing values, where C to G base editing is graphically shown by dotted bars (G) going to filled bars (C), as sequencing was performed on the DNA strand opposite of the strand containing the edited C.

FIG. 20 shows total editing percentages at the RNF2 site with additional C to G base editors (BE3; BE3_UdgX; BE3_REV7; and SMUG1, where BE3 and BE3_UdgX are repeated from FIG. 16) in UDG^{-/-} Hap1 cells. The top panel shows the raw editing values. The bottom panel shows a graphical representation of the raw editing values, where C to G base editing is graphically shown by dotted bars (G) going to filled bars (C), as sequencing was performed on the DNA strand opposite of the strand containing the edited C.

FIG. 21 shows the editing specificity ratio at the RNF2 site with various C to G base editors (BE3; BE3_UdgX; BE3_UdgX*; BE3_REV7; BE2_UDG; BE3_UDG BE2_UdgX_On; BE3_UdgX_On; and SMUG1) in UDG^{-/-} Hap1 cells. The top panel shows the total percentage of edits and the ratio of edits that have been made from G to A, C, or T. The bottom panel is a graphical representation of the specificity ratio values.

FIG. 22 shows total editing percentages at the FANCF site in UDG^{-/-} Hap1 cells using seven base editors (BE3; BE3_UdgX; BE3_UdgX*; BE2_UdgX_On; BE3_UdgX_On; BE2_UDG; and BE3_UDG). Raw editing values are shown in the left panel. The panel on the right shows a graphical representation of the raw editing values, where C to G base editing is graphically shown by filled bars (C) going to dotted bars (G).

FIG. 23 shows total editing percentages at the FANCF site with additional C to G base editors (BE3; BE3_UdgX; BE3_REV7; and SMUG1, where BE3 and BE3_UdgX are repeated from FIG. 19) in UDG^{-/-} Hap1 cells. The top panel shows the raw editing values. The bottom panel shows a graphical representation of the raw editing values, where C to G base editing is graphically shown by filled bars (C) going to dotted bars (G).

FIG. 24 shows the editing specificity ratio at the FANCF site with various C to G base editors (BE3; BE3_UdgX; BE3_UdgX*; BE3_REV7; BE2_UDG; BE3_UDG BE2_UdgX_On; BE3_UdgX_On; and SMUG1) in UDG^{-/-} Hap1 cells. The top panel shows the total percentage of edits and the ratio of edits that have been made from C to A, G, or T. The bottom panel is a graphical representation of the specificity ratio values.

FIG. 25 shows total editing percentages at the HEK2 site with various C to G base editors (BE3; BE3_UdgX; BE2_UNG; BE3_UNG; BE2UdgX_On; BE3UdgX_On; and SMUG1) in REV1^{-/-} Hap1 cells. The top panel shows the raw editing values. The bottom panel shows a graphical representation of the raw editing values, where C to G base editing is graphically shown by dotted bars (G) going to filled bars (C), as sequencing was performed on the DNA strand opposite of the strand containing the edited C.

FIG. 26 shows editing specificity ratio at the HEK2 site with various C to G base editors (BE3; BE3_UdgX; BE2_UNG; BE3_UNG; BE2UdgX_On; BE3UdgX_On; and SMUG1) in REV1^{-/-} Hap1 cells. The top panel shows the total percentage of edits and the ratio of edits that have been made from G to A, C, or T. The bottom panel is a graphical representation of the specificity ratio values.

FIG. 27 shows total editing percentages at the RNF2 site with various C to G base editors (BE3; BE3_UdgX; BE2_UNG; BE3_UNG; BE2UdgX_On; BE3UdgX_On; and SMUG1) in REV1^{-/-} Hap1 cells. The top panel shows the raw editing values. The bottom panel shows a graphical representation of the raw editing values, where C to G base editing is graphically shown by dotted bars (G) going to filled bars (C), as sequencing was performed on the DNA strand opposite of the strand containing the edited C.

FIG. 28 shows editing specificity ratio at the RNF2 site with various C to G base editors (BE3; BE3_UdgX; BE2_UNG; BE3_UNG; BE2UdgX_On; BE3UdgX_On; and SMUG1) in REV1^{-/-} Hap1 cells. The top panel shows the total percentage of edits and the ratio of edits that have been made from G to A, C, or T. The bottom panel is a graphical representation of the specificity ratio values.

FIG. 29 shows total editing percentages at the FANCF site with various C to G base editors (BE3; BE3_UdgX; BE2_UNG; BE3_UNG; BE2UdgX_On; BE3UdgX_On; and SMUG1) in REV1^{-/-} Hap1 cells. The top panel shows the raw editing values. The bottom panel shows a graphical representation of the raw editing values, where C to G base editing is graphically shown by filled bars (C) going to dotted bars (G).

FIG. 30 shows editing specificity ratio at the FANCF site with various C to G base editors (BE3; BE3_UdgX; BE2_UNG; BE3_UNG; BE2UdgX_On; BE3UdgX_On; and SMUG1) in REV1^{-/-} Hap1 cells. The top panel shows the total percentage of edits and the ratio of edits that have been made from C to A, G, or T. The bottom panel is a graphical representation of the specificity ratio values.

FIG. 31 shows a graphical representation of the raw editing values for the percent of total editing at the HEK2, RNF2, and FANCF sites using the indicated C to G base editors.

FIG. 32 shows a graphical representation of the specificity ratio for the percent of total editing at the HEK2, RNF2, and FANCF sites.

FIG. 33 shows a schematic illustrating an approach to increase in the incorporation of C opposite an abasic site, for C to G base editing. If the preference for C integration opposite an abasic site is increased, for example by using a polymerase (e.g., a translesion polymerase), the total C to G base editing will also be increased.

FIG. 34 shows a schematic illustrating an approach to increase in the incorporation of C opposite an abasic site, for C to G base editing. If the preference for C integration opposite an abasic site is increased, for example by incorporating a translesion polymerase into the base editor, the total C to G base editing may also be increased.

FIG. 35 shows a schematic illustrating the different polymerases that can be used in the C to G base editing approach of FIGS. 33 and 34.

FIG. 36 shows a schematic (on the left) illustrating an exemplary C to T base editor (e.g., BE3), which contains a uracil glycosylase inhibitor (UGI), a Cas9 domain (e.g., nCas9), and a cytidine deaminase. On the right is a schematic illustrating a C to G base editor, which contains a translesion polymerase, a Cas9 domain (e.g., nCas9), and a cytidine deaminase.

FIG. 37 shows base editing at the HEK2 site in WT cells using base editors tethered to REV1, Pol Kappa, Pol Eta, and Pol Iota. C to G editing is graphically shown by dotted bars (G) going to filled bars (C) in the graphical representation on the right panel. Pol Kappa tethering dramatically increases the efficiency of C to G editing. Raw editing values are shown on the left panel.

FIG. 38 shows base editing at the RNF2 site in WT cells using base editors tethered to REV1, Pol Kappa, Pol Eta, and Pol Iota. C to G editing is graphically shown by dotted bars (G) going to filled bars (C) in the graphical representation on the right panel. Pol Kappa tethering dramatically increases the efficiency of C to G editing. Raw editing values are shown on the left panel.

FIG. 39 shows base editing at the FANCF site in WT cells using base editors tethered to REV1, Pol Kappa, Pol Eta, and Pol Iota. C to G editing is graphically shown by filled bars (C) going to dotted bars (G) in the graphical representation on the right panel. Pol Kappa tethering dramatically increases the efficiency of C to G editing. Raw editing values are shown on the left panel.

FIG. 40 shows a schematic (on the left) illustrating an exemplary C to G base editor, which contains a uracil DNA glycosylase (UDG), a translesion polymerase, a Cas9 domain (e.g., nCas9), and a cytidine deaminase. On the right is a schematic illustrating a C to G base editor, which contains a translesion polymerase, a Cas9 domain (e.g., nCas9), and a base excision enzyme (e.g., a UDG variant capable of excising a C or T residue).

FIG. 41 shows C to G base editing using the base editor illustrated in the left panel of FIG. 40 (base editor containing a uracil DNA glycosylase (UDG), a translesion polymerase, a Cas9 domain, and a cytidine deaminase) at HEK2, RNF2, and FANCF sites using either Pol Kappa or Pol Iota tethered constructs. C to G editing is graphically shown by dotted bars (G) going to filled bars (C) for HEK2 and RNF2, and filled bars (C) going to dotted bars (G) for FANCF.

FIG. 42 shows base editing at the HEK2 site in WT cells using base editors tethered to either Pol Kappa, Pol Eta, Pol Iota, and REV1, which are shown in the right panel of FIG. 40 (base editor containing a translesion polymerase, a Cas9 domain, and base excision enzyme (UDG 147) which excises T). The amount C to G is graphically illustrated at specific residues in the HEK2 site. UDG 147 is a UDG variant that directly removes T.

FIG. 43 shows base editing at the RNF2 site in WT cells using base editors tethered to either Pol Kappa, Pol Eta, Pol Iota, and REV1, which are shown in the right panel of FIG. 40 (base editor containing a translesion polymerase, a Cas9 domain, and base excision enzyme (UDG 147) which excises T). The amount C to G is graphically illustrated at specific residues in the HEK2 site. UDG 147 is a UDG variant that directly removes T.

FIG. 44 shows base editing at the FANCF site in WT cells using base editors tethered to either Pol Kappa, Pol Eta, Pol Iota, and REV1, which are shown in the right panel of FIG. 40 (base editor containing a translesion polymerase, a Cas9 domain, and base excision enzyme (UDG 147) which excises T). The amount C to G is graphically illustrated at specific residues in the HEK2 site. UDG 147 is a UDG variant that directly removes T.

FIG. 45 shows base editing at the HEK2 site in WT cells using base editors tethered to either Pol Kappa, Pol Eta, Pol Iota, and REV1, which are shown in the right panel of FIG. 40 (base editor containing a translesion polymerase, a Cas9 domain, and base excision enzyme (UDG 204) which excises C). The amount C to G is graphically illustrated at specific residues in the HEK2 site. UDG 204 is a UDG variant that directly removes C.

FIG. 46 shows base editing at the RNF2 site in WT cells using base editors tethered to either Pol Kappa, Pol Eta, Pol Iota, and REV1, which are shown in the right panel of FIG. 40 (base editor containing a translesion polymerase, a Cas9 domain, and base excision enzyme (UDG 204) which excises C). The amount C to G is graphically illustrated at specific residues in the HEK2 site. UDG 204 is a UDG variant that directly removes C.

FIG. 47 shows base editing at the FANCF site in WT cells using base editors tethered to either Pol Kappa, Pol Eta, Pol Iota, and REV1, which are shown in the right panel of FIG. 40 (base editor containing a translesion polymerase, a Cas9 domain, and base excision enzyme (UDG 204) which excises C). The amount C to G is graphically illustrated at specific residues in the HEK2 site. UDG 204 is a UDG variant that directly removes C.

FIG. 48 shows a schematic illustrating a role of MSH2 in base repair, where MSH2 may facilitate the conversion of a uracil (U) to a cytosine (C) in DNA.

FIG. 49 shows base editing at the HEK2 site in MSH2−/− cells using six base editors (BE3; BE3_UdgX; BE3_UdgX*; BE2_UdgX_On; BE3_UdgX_On; and BE3_UDG). Raw editing values are shown in the left panel. The panel on the right shows a graphical representation of the raw editing values, where C to G base editing is graphically shown by dotted bars (G) going to filled bars (C).

FIG. 50 shows base editing at the RNF2 site in MSH2−/− cells using six base editors (BE3; BE3_UdgX; BE3_UdgX*; BE2_UdgX_On; BE3_UdgX_On; and BE3_UDG). Raw editing values are shown in the left panel. The panel on the right shows a graphical representation of the raw editing values, where C to G base editing is graphically shown by dotted bars (G) going to filled bars (C).

FIG. 51 shows base editing at the FANCF site in MSH2−/− cells using six base editors (BE3; BE3_UdgX; BE3_UdgX*; BE2_UdgX_On; BE3_UdgX_On; and BE3_UNG). Raw editing values are shown in the left panel. The panel on the right shows a graphical representation of the raw editing values, where C to G base editing is graphically shown by filled bars (C) going to dotted bars (G).

FIG. 52 shows a schematic illustrating a base editing approach where a C to G base editor containing a UDG (or a UDG variant), a Cas9 (e.g., nCas9) domain, and a cytidine deaminase is expressed in trans with a translesion polymerase.

FIG. 53 shows base editing at the HEK2 site in HEK293 cells using five base editors (BE3; BE3_UdgX; BE3_UdgX*; BE2_UdgX_On; and BE3_UDG) expressed, in trans, with various polymerases (Pol Kappa, Pol Eta, Pol Iota, REV1, Pol Beta, and Pol Delta). C to G base editing is graphically shown by dotted bars (G) going to filled bars (C).

FIG. 54 shows base editing at the RNF2 site in HEK293 cells using five base editors (BE3; BE3_UdgX; BE3_UdgX*; BE2_UdgX_On; and BE3_UDG) expressed, in trans, with various polymerases (Pol Kappa, Pol Eta, Pol Iota, REV1, Pol Beta, and Pol Delta). C to G base editing is graphically shown by dotted bars (G) going to filled bars (C).

FIG. 55 shows base editing at the FANCF site in HEK293 cells using five base editors (BE3; BE3_UdgX; BE3_UdgX*; BE2_UdgX_On; and BE3_UDG) expressed, in trans, with various polymerases (Pol Kappa, Pol Eta, Pol Iota, REV1, Pol Beta, and Pol Delta). C to G base editing is graphically shown by filled bars (C) going to dotted bars (G).

FIGS. 56A-56C show development of prototype C•G-to-G•C base editors. FIG. 56A: Potential pathway for C•G-to-G•C conversion. FIG. 56B: C•G-to-G•C editing outcomes in HEK293T cells for C-terminal fusions of DNA glycosylases to BE4B (AC, APOBEC1 cytidine deaminase-Cas9 nickase). FIG. 56C: Different fusion protein architectures lead to different C•G-to-G•C editing properties in HEK293T cells at the HEK3 locus for the Apo-UdgX-Cas9n (AXC) architecture. Values and error bars reflect the mean and standard deviation of three biological replicates, shown as individual data points. HEK2=HEK site 2; HEK3=HEK site 3; HEK4=HEK site 4. C4, C6, and similar annotations indicate the in-window target nucleotides where the SpCas9 PAM is at positions 21-23.

FIGS. 57A-57D show a CRISPRi knockdown screen across 476 genes enriched for those with roles in DNA repair to identify candidate regulators of C•G-to-G•C editing. FIG. 57A: Schematic of screen design. FIG. 57B: Summary of base editing outcomes in BE4B (also AC) screen. Bottom left—all editing outcomes containing only point mutations present at >=1% frequency for non-targeting CRISPRi guide RNAs. Line plots above the individual outcomes show the total editing frequency (black line) and the frequencies of each single base edit (C-to-T=“★”, C-to-G=“Δ”, C-to-A=“⋆”, and G-to-C=“⋄”) at each position. Line plots to the right show frequencies of outcomes for specific CRISPRi guide RNAs (blue−average of all non-targeting guide+/−standard deviation across individual non-targeting guide RNAs; top 2 most active UNG guide RNAs are labeled according to the legend provided). Heatmaps show log 2 fold changes in outcome frequencies for top 2 UNG guide RNAs relative to non-targeting guide RNAs. FIG. 57C: Log₂ fold changes in frequency of outcomes containing C-to-T or C-to-G edits for each CRISPRi guide compared to non-targeting guide RNAs. Upper left—comparison of changes in C-to-T editing between two biological replicates. Lower right—comparison of changes in C-to-G editing between replicates. Upper right—comparison of changes in C-to-G editing to changes in C-to-T editing in replicate 1. All guide RNAs with at least 500 recovered UMIs in each replicate are plotted. Blue dots: individual non-targeting guide RNAs, orange dots: UNG guide RNAs, green dots: ASCC3 guide RNAs, red dots: RFWD3 guide RNAs, grey dots: all other guide RNAs. FIG. 57D: Effects of gene knockdown on relative C-to-G editing frequencies in BE4B screen. Each dot represents a gene, with the x-value representing the average of the two strongest Log₂ fold changes in normalized C-to-G editing for guide RNAs targeting the gene from the average of all non-targeting guide RNAs, and the y-value representing a gene-level p-value summarizing the combined statistical significance of all guide RNAs targeting each gene (two-sided, uncorrected for multiple comparisons). Rep.=replicate.

FIGS. 58A-58B show the effect of varying the cytidine deaminase and Cas9 components of CGBEs on C•G-to-G•C editing outcomes in HEK293T cells. FIG. 58A: C•G-to-G•C editing outcomes for catalytically impaired, narrow-window cytidine deaminases show higher editing purity at HEK2 and RNF2. FIG. 58B: C•G-to-G•C editing outcomes for high-fidelity Cas9 variants show altered editing windows and improved CGBE performance at some positions. “Cas9” represents the Cas9 D10A nickase variant of each Cas effector. Values and error bars reflect the mean and standard deviation of three biological replicates, shown as individual data points. HEK2=HEK site 2; HEK3=HEK site 3; HEK4=HEK site 4. C4, C6, and similar annotations indicate the in-window target nucleotides where the SpCas9 PAM is at positions 21-23.

FIGS. 59A-59B show that novel engineered CGBEs with various DNA repair proteins, deaminases, Cas proteins, and architectures offer diverse editing performance on different target sites. FIG. 59A: C•G-to-G•C editing performance of CGBEs at eight genomic loci in HEK293T cells. FIG. 59B: Further characterization of C•G-to-G•C editing outcomes for 12 variants from FIG. 59A at various genomic loci in HEK293T cells. Values and error bars reflect the mean and standard deviation of three biological replicates. HEK2=HEK293T cells site 2; HEK3=HEK293T cells site 3; HEK4=HEK293T cells site 4. C nucleotide annotations indicate the target nucleotide positions in the protospacer, where the SpCas9 PAM is at positions 21-23.

FIGS. 60A-60I show target library characterization and machine learning modeling of 10 CGBE variants. FIG. 60A: Overview of genome-integrated target library assay. Libraries of 12,000 or 4,000 pairs of sgRNAs and corresponding target sites are integrated into the genomes of mammalian cells using Tol2 transposase and treated with base editors. Edited cells are enriched by antibiotic selection, and library cassettes are amplified for high-throughput sequencing. FIG. 60B: Base editing windows. Values are C•G-to-G•C editing efficiencies normalized to a maximum of 100. The protospacer is at positions 1-20, with the SpCas9 PAM at positions 21-23. All data are in mES cells except for eA3A-nCas9, which is in HEK293T cells. FIG. 60C: C•G-to-G•C editing purity in the comprehensive context library in mES cells. Box plots indicate median and interquartile range, whiskers indicate extrema, and black dots indicate mean. Two-sided Welch's T-test*P≤5.1×10-9. FIG. 60D: Heatmap of observed C•G-to-G•C purities by CGBE in target contexts from the comprehensive context library in mES cells. Black nucleotides indicate the cytosine for which purity is calculated. Target sites were sorted by outcome variance and manually selected. FIG. 60E: Clustering of CGBEs based on measured C•G-to-G•C purity in core window cytosines across the comprehensive context library in mESCs. Values are Pearson correlation. FIG. 60F: Purity of editing outcomes across core window nucleotides in the comprehensive context library, ranked by C•G-to-G•C purity, averaged across CGBEs in mESCs. Trend lines and shading show the rolling mean and standard deviation across 1% intervals. FIG. 60G: Representative sequence motifs for editing efficiency and C•G-to-G•C purity from logistic regression models. The sign of each learned weight indicates a contribution above (positive sign) or below (negative sign) the mean activity. Logo opacity is proportional to the motif's Pearson's R on held-out sequence contexts. FIG. 60H: Observed C•G-to-G•C purity across CGBEs in mESCs compared to CGBE-Hive predictions. Trend lines and shading show the rolling mean and standard deviation. FIG. 60I: Sequence motifs for C•G-to-G•C editing yield.

FIGS. 61A-61F show target library characterization and machine learning modeling of CGBE variants. FIG. 61A: Observed C-to-G purity by CGBE at SNVs predicted to have >80% C-to-G purity. Box plot indicates median and interquartile range, and whiskers indicate extrema. FIG. 61B: Observed number of disease-related sgRNA-target pairs corrected at varying genotype precision and amino acid precision thresholds by various strategies for selecting CGBEs.. FIG. 61C: Comparison of predicted versus observed correction yield of disease-related transversion SNVs in mES cells. Trend lines and shading show the rolling mean and standard deviation. FIG. 61D: Comparison of predicted versus observed correction precision of disease-related transversion SNVs in mES cells. Trend lines and shading show the rolling mean and standard deviation. FIG. 61E: Observed number of sgRNA-target pairs containing disease-related transversion SNVs corrected at various thresholds for genotype and amino acid precision. FIG. 61F: Installation of disease-associated SNPs using CGBEs.

FIGS. 62A-62D show that HAP1 cells lacking UNG, APE1, REV1, or MLH1 show minimal differences in C•G-to-G•C editing outcomes. C•G-to-G•C editing yield and product purity of BE1 (nuclease inactive, no UGIs), BE4B (D10A nickase, no UGIs; also AC) and AXC (APOBEC1-UdgX-Cas9 D10A, the prototype CGBE), in HAP1 knockout haploid human cell lines lacking (FIG. 62A) UNG, (FIG. 62B) APE1, (FIG. 62C) REV1, and (FIG. 62D) MLH1. Values and error bars reflect the mean and standard deviation of three biological replicates, shown as individual data points, except HEK2 editing in REV1-cells shows two biological replicates. HEK2=HEK293T cells site 2; HEK3=HEK293T cells site 3; HEK4=HEK293T cells site 4. C4, C6, and similar annotations indicate the in-window target nucleotides where the SpCas9 PAM is at positions 21-23.

FIGS. 63A-63B show the effects of polymerase or GFP fusions on C•G-to-G•C editing outcomes. FIG. 63A: C•G-to-G•C editing outcomes in HEK293T cells using N-terminal polymerase fusions to AXC (Polymerase-AXC). GFP-AXC and AXC are shown as controls. FIG. 63B: C•G-to-G•C editing outcomes in HEK293T cells using C-terminal polymerase fusions to AXC (AXC-Polymerase). AXC-GFP is shown as a control with AXC reproduced from FIG. 63A for ease of comparison. C•G-to-G•C editing yield is shown on the x-axis and product purity is shown on the y-axis. Window position annotations indicate the in-window target nucleotides where the SpCas9 PAM is at positions 21-23. Values and error bars reflect the mean and standard deviation of three biological replicates. HEK2=HEK293T cells site 2; HEK3=HEK293T cells site 3; HEK4=HEK293T cells site 4.

FIGS. 64A-64C show additional CRISPRi screen outcomes. FIG. 64A: Summary of base editing outcomes in BE1 screen. Bottom left: all editing outcomes containing only point mutations present at >1% frequency for non-targeting control CRISPRi guide RNAs, ordered by frequency. Line plots above the individual outcomes show the total editing frequency (black line) and the frequencies of each type of single-base mutation (C-to-T=“★”, C-to-G=“Δ”, C-to-A=“⋆”, and G-to-C=“⋄”) at each position. Right: frequencies of outcomes for specific CRISPRi guide RNAs (blue=mean±SD of all non-targeting CRISPRi guide RNAs; orange=the top two most active UNG-targeting CRISPRi guide RNAs). Heatmaps show log₂ fold changes in outcome frequencies for the two most active UNG-targeting CRISPRi guide RNAs relative to non-targeting control CRISPRi guide RNAs. FIG. 64B: Frequency of editing outcome categories in screens. FIG. 64C: Log₂ fold changes in frequency of specific editing outcomes containing C-to-T mutations for UNG-targeting CRISPRi guide RNAs in BE1 (orange) and BE4B (blue) screens. Intervals are 95% Clopper-Pearson binomial confidence intervals for the observed frequencies of each outcome category given the number of UMIs recovered for each CRISPRi guide RNA, converted into log 2 fold changes. Rep.=replicate.

FIGS. 65A-65E show the effects of gene knockdown on editing outcomes by category. Each dot in scatter plots represents a gene, with the x-value representing the average of the two strongest log 2 fold changes in the frequency of the relevant outcome category for CRISPRi guide RNAs targeting that gene compared to the average of all non-targeting guide RNAs, and the y-value representing a gene-level p-value summarizing the combined statistical significance of all guide RNAs targeting each gene. In each panel, the genes with the largest negative (blue) and positive (red) average log 2 fold changes across two replicates that achieve a p-value less than or equal to 10-5 in either replicate are labeled (up to 5 genes labeled). Additional genes with phenotypes referenced in the text are also labeled (black). P-values represent two-sided tests without correction for multiple comparisons. Outcome categories are as follows: FIG. 65A: Outcomes containing any deletion. FIG. 65B: Outcomes containing C•G-to-T•A point mutations, as a fraction of outcomes containing any point mutations. FIG. 65C: Outcomes containing point mutations at specific positions, as a fraction of outcomes containing any point mutation (where the SaCas9 NNGRRT (SEQ ID NO: 223) PAM occupies positions 22-27). The 5 most highly modified positions were included. FIG. 65D: Outcomes containing C•G-to-G•C point mutations, as a fraction of outcomes containing any point mutations. FIG. 65E: Outcomes containing only point mutations. Rep.=replicate.

FIGS. 66A-66B show phenotypes for CRISPRi guide RNAs targeting RECQL and HLTF. FIG. 66A: Effect of RECQL knockdown on editing window in BE4B screens. Bottom left: most frequent point mutation editing outcomes, ordered by average log₂ fold changes in frequency from non-targeting caused by two most active RECQL guide RNAs in replicate 1. Heatmaps show log 2 fold changes from non-targeting guide RNAs. Line plots above outcome diagrams show differences in total editing rates at each position between the top two CRISPRi RECQL guide RNAs and non-targeting guide RNAs. FIG. 66B: Effect of HLTF knockdown on editing window in BE4 (top) and BE1 (bottom) screens. Diagrams show the three most frequent outcomes with an edit at position +3 (where positions 22-27 are the SaCas9 NNGRRT (SEQ ID NO: 223) PAM) for non-targeting CRISPRi guide RNAs. Line plots above outcomes show differences in total editing rates at each position between HLTF guide RNAs and non-targeting guide RNAs. Line plots to the right of outcomes show frequencies of outcomes for specific CRISPRi guide RNAs in replicate 1 (blue (darker shade)=average frequency of each outcome across all non-targeting guide RNAs+/−standard deviation across individual non-targeting guide RNAs; pink (lighter shade)=frequency of each outcome for top 2 HLTF guide RNAs). Heatmaps show log 2 fold changes from non-targeting CRISPRi guide RNAs. Rep.=replicate.

FIGS. 67A-67B show that fusion of proteins to AXC scaffold alters C•G-to-G•C editing outcomes in HEK293T cells. FIG. 67A: C•G-to-G•C editing outcomes of CGBE candidates containing proteins identified in the screen as N-terminal fusions. FIG. 67B: C•G-to-G•C editing outcomes of CGBE candidates containing tandem fusion of proteins identified in the screen. C•G-to-G•C editing yield is shown on the x-axis and product purity is shown on the y-axis. Values and error bars reflect the mean and standard deviation of three biological replicates. Window position annotations indicate the in-window target nucleotides where the SpCas9 PAM is at positions 21-23. HEK2=HEK293T cells site 2; HEK3=HEK293T cells site 3; HEK4=HEK293T cells site 4.

FIG. 68 shows the optimization of linkers between CGBE components. C•G-to-G•C editing outcomes for CGBE candidates with 1-aa, 32-aa, or 60-aa linkers. Values and error bars reflect the mean and standard deviation of three biological replicates, shown as individual data points. HEK2=HEK293T cells site 2; HEK3=HEK293T cells site 3; HEK4=HEK293T cells site 4. C4, C6, and similar annotations indicate the in-window target nucleotides where the SpCas9 PAM is at positions 21-23.

FIG. 69 shows that split-intein and non-split CGBE variants edit with similar yield and product purity. C•G-to-G•C editing outcomes for split-intein (light bars) and non-split (dark bars) CGBE variants tested in HEK293T cells at five genomic loci. Values and error bars reflect the mean and standard deviation of three biological replicates, shown as individual data points. HEK2=HEK293T cells site 2; HEK3=HEK293T cells site 3; HEK4=HEK293T cells site 4. C4, C6, and similar annotations indicate the in-window target nucleotides where the SpCas9 PAM is at positions 21-23.

FIGS. 70A-70B show performance of CGBE variants in K562, U2OS, and HeLa cells. C•G-to-G•C editing outcomes in K562 cells (left column), U2OS cells (middle column), and HeLa cells (right column) at six target cytosines across five genomic loci. Editor identities are depicted at the bottom of the figure. C•G-to-G•C editing yield is shown on the x-axis and product purity is shown on the y-axis. Window position annotations indicate the in-window target nucleotides where the SpCas9 PAM is at positions 21-23. Values and error bars reflect the mean and standard deviation of three biological replicates. HEK2=HEK293T cells site 2; HEK3=HEK293T cells site 3.

FIG. 71 shows CGBE activity using Cas9-NG. C•G-to-G•C editing outcomes in HEK293T cells using CGBE variants containing Cas9-NG at eight target cytosines across seven genomic loci. C•G-to-G•C editing yield is shown on the x-axis and product purity is shown on the y-axis. Values and error bars reflect the mean and standard deviation of three biological replicates. Window position annotations indicate the in-window target nucleotides where the SpCas9 PAM is at positions 21-23. HEK2=HEK293T cells site 2; HEK3=HEK293T cells site 3; HEK4=HEK293T cells site 4; HEK4.1=HEK293T cells site 4.1.

FIG. 72 shows on-target CGBE editing profiles for off-target analyses. C•G-to-G•C editing outcomes in HEK293T cells using nicking CGBEs at eight target cytosines across seven genomic loci). Editor identities are depicted at the bottom of the figure. C•G-to-G•C editing yield is shown on the x-axis and product purity is shown on the y-axis. Values and error bars reflect the mean and standard deviation of three biological replicates. Window position annotations indicate the in-window target nucleotides where the SpCas9 PAM is at positions 21-23. HEK2=HEK293T cells site 2; HEK3=HEK293T cells site 3; HEK4=HEK293T cells site 4; HEK4.1=HEK293T cells site 4.1.

FIGS. 73A-73D show transversion-enriched SNV library analysis. FIG. 73A: Heatmap of observed C•G-to-G•C purities by CGBE variants in target contexts from the transversion-enriched SNV library in mES cells. Underlined nucleotides indicate the cytosine for which purity is calculated. Target sites were sorted by outcome variance and manually selected. FIG. 73B: Replicate consistency statistics. FIG. 73C: Scatter plots of base editing efficiency between experimental replicates. Each point represents a single target site. FIG. 73D: Scatter plots of editing purities between experimental replicates. Each point represents a unique editing pattern in a target site. Scatter plot is plotted across 30 library members.

FIG. 74 shows a comparison of CGBEs developed herein with recently described CGBEs. C•G-to-G•C editing outcomes for CGBEs reported in this study compared with that of mini CGBE1¹⁴, CGBE1¹⁴, APO1-nCas9-UNG¹⁵, and APO1-nCas9-XRCC1¹¹ at 11 different target cytidines across eight genomic loci. C•G-to-G•C editing yield is shown on the x-axis and product purity is shown on the y-axis. Values and error bars reflect the mean and standard deviation of three biological replicates. Window position annotations indicate the in-window target nucleotides where the SpCas9 PAM is at positions 21-23. HEK2=HEK293T cells site 2; HEK3=HEK293T cells site 3; HEK4=HEK293T cells site 4; HEK4.1=HEK293T cells site 4.1.

FIGS. 75A-75B show a comparison of prime editing and CGBE editing outcomes. FIG. 75A: C•G-to-G•C editing outcomes in HEK293T cells using prime editor 2 (PE2) to identify the best-performing pegRNA to make six different edits at four genomic loci (HEK site 3, FANCF, RNF2, and HBBa). FIG. 75B: Comparison of CGBE variants with PE2 and prime editor 3 (PE3) editors at four genomic loci. PE3 editors use an additional sgRNA to nick the non-edited DNA strand. Values and error bars reflect the mean and standard deviation of three biological replicates. C•G-to-G•C editing yield is shown on the x-axis and product purity is shown on the y-axis in FIG. 75B. HEK3=HEK site 3. C4, C6, and similar annotations indicate the in-window target nucleotides where the SpCas9 PAM is at positions 21-23.

FIGS. 76A-76B show off-target DNA editing activities of CGBEs. CGBE activity at 13 off-target loci. Values and error bars reflect the mean and standard deviation of three biological replicates. HEK2=HEK293T cells site 2; HEK3=HEK293T cells site 3; HEK4=HEK293T cells site 4. X=UdgX, D2=POLD2, RB=RBMX, 689=Anc689, HF=HF-nCas9, eA3A*=eA3A T31A.

### DEFINITIONS

As used herein and in the claims, the singular forms “a,” “an,” and “the” include the singular and the plural unless the context clearly indicates otherwise. Thus, for example, a reference to “an agent” includes a single agent and a plurality of such agents.

The term “deaminase” or “deaminase domain,” as used herein, refers to a protein or enzyme that catalyzes a deamination reaction. In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

The term “base editor (BE),” or “nucleobase editor (NBE)” refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. In some embodiments, the base editor is capable of deaminating a cytosine (C) in DNA. In some embodiments, the base editor is capable of excising a base within a DNA molecule. In some embodiments, the base editor is capable of excising an adenine, guanine, cytosine, thymine or uracil within a nucleic acid (e.g., DNA or RNA) molecule. In some embodiments, the base editor is a protein (e.g., a fusion protein) comprising a nucleic acid programmable DNA binding protein (napDNAbp) fused to a cytidine deaminase. In some embodiments, the base editor is fused to a uracil binding protein (UBP), such as a uracil DNA glycosylase (UDG). In some embodiments, the base editor is fused to a nucleic acid polymerase (NAP) domain. In some embodiments, the NAP domain is a translesion DNA polymerase. In some embodiments, the base editor comprises a napDNAbp, a cytidine deaminase and a UBP (e.g., UDG). In some embodiments, the base editor comprises a napDNAbp, a cytidine deaminase and a nucleic acid polymerase (e.g., a translesion DNA polymerase). In some embodiments, the base editor comprises a napDNAbp, a cytidine deaminase, a UBP (e.g., UDG), and a nucleic acid polymerase (e.g., a translesion DNA polymerase).

In some embodiments, the napDNAbp of the base editor is a Cas9 domain. In some embodiments, the base editor comprises a Cas9 protein fused to a cytidine deaminase. In some embodiments, the base editor comprises a Cas9 nickase (nCas9) fused to a cytidine deaminase. In some embodiments, the Cas9 nickase comprises a D10A mutation and comprises a histidine at residue 840 of SEQ ID NO: 6, or a corresponding mutation in any Cas9 provided herein, such as any one of SEQ ID NOs: 4-26, which renders Cas9 capable of cleaving only one strand of a nucleic acid duplex. In some embodiments, the base editor comprises a nuclease-inactive Cas9 (dCas9) fused to a cytidine deaminase.

In some embodiments, the dCas9 domain comprises a D10A and a H840A mutation of SEQ ID NO: 6, or a corresponding mutation in any Cas9 provided herein, such as any one of SEQ ID NOs: 4-26, which inactivates the nuclease activity of the Cas9 protein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017 and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28; 152(5):1173-83 (2013), each of which are incorporated by reference herein).

In some embodiments, a base editor is a macromolecule or macromolecular complex that results primarily (e.g., more than 80%, more than 85%, more than 90%, more than 95%, more than 99%, more than 99.9%, or 100%) in the conversion of a nucleobase in a polynucleic acid sequence into another nucleobase (i.e., a transition or transversion) using a combination of 1) a nucleotide-, nucleoside-, or nucleobase-modifying enzyme and 2) a nucleic acid binding protein that can be programmed to bind to a specific nucleic acid sequence.

In some embodiments, the base editor comprises a DNA binding domain (e.g., a programmable DNA binding domain such as a dCas9 or nCas9) that directs it to a target sequence. In some embodiments, the base editor comprises a nucleobase modifying enzyme fused to a programmable DNA binding domain (e.g., a dCas9 or nCas9). A “nucleobase modifying enzyme” is an enzyme that can modify a nucleobase and convert one nucleobase to another (e.g., a cytidine deaminase). In some embodiments, the base editor may target cytosine (C) bases in a nucleic acid sequence and convert the C to guanine (G) base. In some embodiments, the C to G editing is carried out in part by a deaminase, e.g., a cytidine deaminase.

Base editors that deaminate a C, in some embodiments, comprise a cytidine deaminase. A “cytidine deaminase” refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U nucleobase change. In the context of a gene, such a nucleotide change, or mutation, may in turn lead to an amino acid change in the protein, which may affect the protein's function, e.g., loss-of-function or gain-of-function. In some embodiments, the CGBE comprises a dCas9 or nCas9 fused to a cytidine deaminase. In some embodiments, the cytidine deaminase domain is fused to the N-terminus of the dCas9 or nCas9. In some embodiments, the base editor further comprises a domain that inhibits uracil glycosylase, and/or a nuclear localization signal. Such base editors have been described in the art, e.g., in Rees & Liu, Nat Rev Genet. 2018; 19(12):770-788 and Koblan et al., Nat Biotechnol. 2018; 36(9):843-846; as well as. U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2018/165629, published Sep. 13, 2018; International Publication No. WO 2019/023680, published Jan. 31, 2019; International Publication No. WO 2019/226593, published Nov. 28, 2019; International Publication No. WO 2018/0176009, published Sep. 27, 2018, International Publication No. WO 2020/041751, published Feb. 27, 2020; International Publication No. WO 2020/051360, published Mar. 12, 2020; International Publication No. WO 2020/102659, published May 22, 2020; International Publication No. WO 2020/086908, published Apr. 30, 2020; International Publication No. WO 2020/181180, published Sep. 10, 2020; International Publication No. WO 2020/181195, published Sep. 10, 2020; International Publication No. WO 2020/214842, published Oct. 22, 2020; International Publication No. WO 2020/092453, published May 7, 2020; International Publication No. WO2020/236982, published Nov. 26, 2020; International Application No. PCT/US2020/624628, filed Nov. 25, 2020; International Publication No. WO 2021/108717, published Jun. 3, 2021, and International Application No. PCT/US2021/016827, which published as International Publication No. WO 2021/158921 on Aug. 12, 2021, the contents of each of which are incorporated herein by reference in their entireties.

The term “base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

The term “linker,” as used herein, refers to a bond (e.g., covalent bond), chemical group, or a molecule linking two molecules or moieties, e.g., two domains of a fusion protein, such as, for example, a nuclease-inactive Cas9 domain and a nucleic acid-editing domain (e.g., an cytidine deaminase). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, a linker comprises the amino acid sequence SGSETPGTSESATPES (SEQ ID NO: 102), which may also be referred to as the XTEN linker. In some embodiments, a linker comprises the amino acid sequence SGGS (SEQ ID NO: 103). In some embodiments, a linker comprises (SGGS)ₙ(SEQ ID NO: 103), (GGGS)ₙ(SEQ ID NO: 104), (GGGGS)ₙ (SEQ ID NO: 105), (G)ₙ (SEQ ID NO: 121), (EAAAK)ₙ (SEQ ID NO: 106), (GGS)ₙ (SEQ ID NO: 122), SGSETPGTSESATPES (SEQ ID NO: 102), (XP)ₙ motif (SEQ ID NO: 123), SGGSSGSETPGTSESATPESSGGS (SEQ ID NO: 107), SGGSSGGSSGSETPGTSESATPESSGGSSGGS (SEQ ID NO: 108), GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 109), SGGSGGSGGS (SEQ ID NO: 120), or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “uracil binding protein” or “UBP,” as used herein, refers to a protein that is capable of binding to uracil. In some embodiments, the uracil binding protein is a uracil modifying enzyme. In some embodiments, the uracil binding protein is a uracil base excision enzyme. In some embodiments, the uracil binding protein is a uracil DNA glycosylase (UDG). In some embodiments, a uracil binding protein binds uracil with an affinity that is at least 1%, 2%, 3%, 5%, 10%, 15%, 20%, 30%, 40%, 50%, 60%, 70%, 80%, 90%, or at least 95% of the affinity that a wild type UDG (e.g., a human UDG) binds to uracil.

The term “base excision enzyme” or “BEE,” as used herein, refers to a protein that is capable of removing a base (e.g., A, T, C, G, or U) from a nucleic acid molecule (e.g., DNA or RNA). In some embodiments, a BEE is capable of removing a cytosine from DNA.

In some embodiments, a BEE is capable of removing a thymine from DNA. Exemplary BEEs include, without limitation UDG Tyr147Ala, and UDG Asn204Asp as described in Sang et al., “A Unique Uracil-DNA binding protein of the uracil DNA glycosylase superfamily,” Nucleic Acids Research, Vol. 43, No. 17 2015; the entire contents of which are hereby incorporated by reference.

The term “nucleic acid polymerase” or “NAP,” refers to an enzyme that synthesizes nucleic acid molecules (e.g., DNA and RNA) from nucleotides (e.g., deoxyribonucleotides and ribonucleotides). In some embodiments, the NAP is a DNA polymerase. In some embodiments, the NAP is a translesion polymerase. Translesion polymerases play a role in mutagenesis, for example, by restarting replication forks or filling in gaps that remain in the genome due to the presence of DNA lesions. Exemplary translesion polymerases include, without limitation, Pol Beta, Pol Lambda, Pol Eta, Pol Mu, Pol Iota, Pol Kappa, Pol Alpha, Pol Delta, Pol Gamma, and Pol Nu.

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. In some embodiments, the NLS is a monopartite NLS. In some embodiments, the NLS is a bipartite NLS. Bipartite NLSs are separated by a relatively short spacer sequence (e.g., from 2-20 amino acids, from 5-15 amino acids, or from 8-12 amino acids). For example, NLS sequences are described in Plank et al., international PCT application, PCT/EP2000/011690, filed Nov. 23, 2000, published as WO 2001/038547 on May 31, 2001; and Kethar, K. M. V., et al., “Application of bioinformatics-coupled experimental analysis reveals a new transport-competent nuclear localization signal in the nucleoptotein of Influenza A virus strain” BMC Cell Biol, 2008, 9: 22; the contents of each of which are incorporated herein by reference for their disclosure of exemplary nuclear localization sequences. In some embodiments, a NLS comprises the amino acid sequence PKKKRKV (SEQ ID NO: 41), MDSLLMNRRKFLYQFKNVRWAKGRRETYLC (SEQ ID NO: 42), KRTADGSEFESPKKKRKV (SEQ ID NO: 43), KRGINDRNFWRGENGRKTR (SEQ ID NO: 44), KKTGGPIYRRVDGKWRR (SEQ ID NO: 45), RRELILYDKEEIRRIWR (SEQ ID NO: 46), or AVSRKRKA (SEQ ID NO: 47).

The term “nucleic acid programmable DNA binding protein” or “napDNAbp” refers to a protein that associates with a nucleic acid (e.g., DNA or RNA), such as a guide nuclic acid, that guides the napDNAbp to a specific nucleic acid sequence. For example, a Cas9 protein can associate with a guide RNA that guides the Cas9 protein to a specific DNA sequence that has complementary to the guide RNA. In some embodiments, the napDNAbp is a class 2 microbial CRISPR-Cas effector. In some embodiments, the napDNAbp is a Cas9 domain, for example a nuclease active Cas9, a Cas9 nickase (nCas9), or a nuclease inactive Cas9 (dCas9). Examples of nucleic acid programmable DNA binding proteins include, without limitation, Cas9 (e.g., dCas9 and nCas9), CasX, CasY, Cpf1, C2c1, C2c2, C2C3, and Argonaute. It should be appreciated, however, that nucleic acid programmable DNA binding proteins also include nucleic acid programmable proteins that bind RNA. For example, the napDNAbp may be associated with a nucleic acid that guides the napDNAbp to an RNA. Other nucleic acid programmable DNA binding proteins are also within the scope of this disclosure, though they may not be specifically listed in this disclosure.

The term “Cas9” or “Cas9 domain” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.

A nuclease-inactivated Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6,7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, the fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or 1300 amino acids in length. In some embodiments, wild type Cas9 corresponds to Cas9 from Streptococcus pyogenes (NCBI Reference Sequence: NC_017053.1, SEQ ID NO: 1 (nucleotide); SEQ ID NO: 4 (amino acid)).

(SEQ ID NO: 1)
ATGGATAAGAAATACTCAATAGGCTTAGATATCGGCACAAATAGC
GTCGGATGGGCGGTGATCACTGATGATTATAAGGTTCCGTCTAAA
AAGTTCAAGGTTCTGGGAAATACAGACCGCCACAGTATCAAAAAA
AATCTTATAGGGGCTCTTTTATTTGGCAGTGGAGAGACAGCGGAA
GCGACTCGTCTCAAACGGACAGCTCGTAGAAGGTATACACGTCGG
AAGAATCGTATTTGTTATCTACAGGAGATTTTTTCAAATGAGATG
GCGAAAGTAGATGATAGTTTCTTTCATCGACTTGAAGAGTCTTTT
TTGGTGGAAGAAGACAAGAAGCATGAACGTCATCCTATTTTTGGA
AATATAGTAGATGAAGTTGCTTATCATGAGAAATATCCAACTATC
TATCATCTGCGAAAAAAATTGGCAGATTCTACTGATAAAGCGGAT
TTGCGCTTAATCTATTTGGCCTTAGCGCATATGATTAAGTTTCGT
GGTCATTTTTTGATTGAGGGAGATTTAAATCCTGATAATAGTGAT
GTGGACAAACTATTTATCCAGTTGGTACAAATCTACAATCAATTA
TTTGAAGAAAACCCTATTAACGCAAGTAGAGTAGATGCTAAAGCG
ATTCTTTCTGCACGATTGAGTAAATCAAGACGATTAGAAAATCTC
ATTGCTCAGCTCCCCGGTGAGAAGAGAAATGGCTTGTTTGGGAAT
CTCATTGCTTTGTCATTGGGATTGACCCCTAATTTTAAATCAAAT
TTTGATTTGGCAGAAGATGCTAAATTACAGCTTTCAAAAGATACT
TACGATGATGATTTAGATAATTTATTGGCGCAAATTGGAGATCAA
TATGCTGATTTGTTTTTGGCAGCTAAGAATTTATCAGATGCTATT
TTACTTTCAGATATCCTAAGAGTAAATAGTGAAATAACTAAGGCT
CCCCTATCAGCTTCAATGATTAAGCGCTACGATGAACATCATCAA
GACTTGACTCTTTTAAAAGCTTTAGTTCGACAACAACTTCCAGAA
AAGTATAAAGAAATCTTTTTTGATCAATCAAAAAACGGATATGCA
GGTTATATTGATGGGGGAGCTAGCCAAGAAGAATTTTATAAATTT
ATCAAACCAATTTTAGAAAAAATGGATGGTACTGAGGAATTATTG
GTGAAACTAAATCGTGAAGATTTGCTGCGCAAGCAACGGACCTTT
GACAACGGCTCTATTCCCCATCAAATTCACTTGGGTGAGCTGCAT
GCTATTTTGAGAAGACAAGAAGACTTTTATCCATTTTTAAAAGAC
AATCGTGAGAAGATTGAAAAAATCTTGACTTTTCGAATTCCTTAT
TATGTTGGTCCATTGGCGCGTGGCAATAGTCGTTTTGCATGGATG
ACTCGGAAGTCTGAAGAAACAATTACCCCATGGAATTTTGAAGAA
GTTGTCGATAAAGGTGCTTCAGCTCAATCATTTATTGAACGCATG
ACAAACTTTGATAAAAATCTTCCAAATGAAAAAGTACTACCAAAA
CATAGTTTGCTTTATGAGTATTTTACGGTTTATAACGAATTGACA
AAGGTCAAATATGTTACTGAGGGAATGCGAAAACCAGCATTTCTT
TCAGGTGAACAGAAGAAAGCCATTGTTGATTTACTCTTCAAAACA
AATCGAAAAGTAACCGTTAAGCAATTAAAAGAAGATTATTTCAAA
AAAATAGAATGTTTTGATAGTGTTGAAATTTCAGGAGTTGAAGAT
AGATTTAATGCTTCATTAGGCGCCTACCATGATTTGCTAAAAATT
ATTAAAGATAAAGATTTTTTGGATAATGAAGAAAATGAAGATATC
TTAGAGGATATTGTTTTAACATTGACCTTATTTGAAGATAGGGGG
ATGATTGAGGAAAGACTTAAAACATATGCTCACCTCTTTGATGAT
AAGGTGATGAAACAGCTTAAACGTCGCCGTTATACTGGTTGGGGA
CGTTTGTCTCGAAAATTGATTAATGGTATTAGGGATAAGCAATCT
GGCAAAACAATATTAGATTTTTTGAAATCAGATGGTTTTGCCAAT
CGCAATTTTATGCAGCTGATCCATGATGATAGTTTGACATTTAAA
GAAGATATTCAAAAAGCACAGGTGTCTGGACAAGGCCATAGTTTA
CATGAACAGATTGCTAACTTAGCTGGCAGTCCTGCTATTAAAAAA
GGTATTTTACAGACTGTAAAAATTGTTGATGAACTGGTCAAAGTA
ATGGGGCATAAGCCAGAAAATATCGTTATTGAAATGGCACGTGAA
AATCAGACAACTCAAAAGGGCCAGAAAAATTCGCGAGAGCGTATG
AAACGAATCGAAGAAGGTATCAAAGAATTAGGAAGTCAGATTCTT
AAAGAGCATCCTGTTGAAAATACTCAATTGCAAAATGAAAAGCTC
TATCTCTATTATCTACAAAATGGAAGAGACATGTATGTGGACCAA
GAATTAGATATTAATCGTTTAAGTGATTATGATGTCGATCACATT
GTTCCACAAAGTTTCATTAAAGACGATTCAATAGACAATAAGGTA
CTAACGCGTTCTGATAAAAATCGTGGTAAATCGGATAACGTTCCA
AGTGAAGAAGTAGTCAAAAAGATGAAAAACTATTGGAGACAACTT
CTAAACGCCAAGTTAATCACTCAACGTAAGTTTGATAATTTAACG
AAAGCTGAACGTGGAGGTTTGAGTGAACTTGATAAAGCTGGTTTT
ATCAAACGCCAATTGGTTGAAACTCGCCAAATCACTAAGCATGTG
GCACAAATTTTGGATAGTCGCATGAATACTAAATACGATGAAAAT
GATAAACTTATTCGAGAGGTTAAAGTGATTACCTTAAAATCTAAA
TTAGTTTCTGACTTCCGAAAAGATTTCCAATTCTATAAAGTACGT
GAGATTAACAATTACCATCATGCCCATGATGCGTATCTAAATGCC
GTCGTTGGAACTGCTTTGATTAAGAAATATCCAAAACTTGAATCG
GAGTTTGTCTATGGTGATTATAAAGTTTATGATGTTCGTAAAATG
ATTGCTAAGTCTGAGCAAGAAATAGGCAAAGCAACCGCAAAATAT
TTCTTTTACTCTAATATCATGAACTTCTTCAAAACAGAAATTACA
CTTGCAAATGGAGAGATTCGCAAACGCCCTCTAATCGAAACTAAT
GGGGAAACTGGAGAAATTGTCTGGGATAAAGGGCGAGATTTTGCC
ACAGTGCGCAAAGTATTGTCCATGCCCCAAGTCAATATTGTCAAG
AAAACAGAAGTACAGACAGGCGGATTCTCCAAGGAGTCAATTTTA
CCAAAAAGAAATTCGGACAAGCTTATTGCTCGTAAAAAAGACTGG
GATCCAAAAAAATATGGTGGTTTTGATAGTCCAACGGTAGCTTAT
TCAGTCCTAGTGGTTGCTAAGGTGGAAAAAGGGAAATCGAAGAAG
TTAAAATCCGTTAAAGAGTTACTAGGGATCACAATTATGGAAAGA
AGTTCCTTTGAAAAAAATCCGATTGACTTTTTAGAAGCTAAAGGA
TATAAGGAAGTTAAAAAAGACTTAATCATTAAACTACCTAAATAT
AGTCTTTTTGAGTTAGAAAACGGTCGTAAACGGATGCTGGCTAGT
GCCGGAGAATTACAAAAAGGAAATGAGCTGGCTCTGCCAAGCAAA
TATGTGAATTTTTTATATTTAGCTAGTCATTATGAAAAGTTGAAG
GGTAGTCCAGAAGATAACGAACAAAAACAATTGTTTGTGGAGCAG
CATAAGCATTATTTAGATGAGATTATTGAGCAAATCAGTGAATTT
TCTAAGCGTGTTATTTTAGCAGATGCCAATTTAGATAAAGTTCTT
AGTGCATATAACAAACATAGAGACAAACCAATACGTGAACAAGCA
GAAAATATTATTCATTTATTTACGTTGACGAATCTTGGAGCTCCC
GCTGCTTTTAAATATTTTGATACAACAATTGATCGTAAACGATAT
ACGTCTACAAAAGAAGTTTTAGATGCCACTCTTATCCATCAATCC
ATCACTGGTCTTTATGAAACACGCATTGATTTGAGTCAGCTAGGA
GGTGACTGA
(SEQ ID NO: 4)
MDKKYSIGLDIGTNSVGWAVITDDYKVPSKKFKVLGNTDRHSIKK
NLIGALLEGSGETAEATRLKRTARRRYTRRKNRICYLQEIFSNEM
AKVDDSFFHRLEESFLVEEDKKHERHPIFGNIVDEVAYHEKYPTI
YHLRKKLADSTDKADLRLIYLALAHMIKFRGHFLIEGDLNPDNSD
VDKLFIQLVQIYNQLFEENPINASRVDAKAILSARLSKSRRLENL
IAQLPGEKRNGLFGNLIALSLGLTPNFKSNFDLAEDAKLQLSKDT
YDDDLDNLLAQIGDQYADLFLAAKNLSDAILLSDILRVNSEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGAYHDLLKIIKDKDFLDNEENEDILEDIVLTLTLFEDRG
MIEERLKTYAHLFDDKVMKQLKRRRYTGWGRLSRKLINGIRDKQS
GKTILDFLKSDGFANRNFMQLIHDDSLTFKEDIQKAQVSGQGHSL
HEQIANLAGSPAIKKGILQTVKIVDELVKVMGHKPENIVIEMARE
NQTTQKGQKNSRERMKRIEEGIKELGSQILKEHPVENTQLQNEKL
YLYYLQNGRDMYVDQELDINRLSDYDVDHIVPQSFIKDDSIDNKV
LTRSDKNRGKSDNVPSEEVVKKMKNYWRQLLNAKLITQRKFDNLT
KAERGGLSELDKAGFIKRQLVETRQITKHVAQILDSRMNTKYDEN
DKLIREVKVITLKSKLVSDFRKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFDSPTVAYSVLVVAKVEKGKSKKLKSVKELLGITIMER
SSFEKNPIDFLEAKGYKEVKKDLIIKLPKYSLFELENGRKRMLAS
AGELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKRYTSTKEVLDATLIHQS
ITGLYETRIDLSQLGGD
(single underline: HNH domain;
double underline: RuvC domain)

In some embodiments, wild type Cas9 corresponds to, or comprises SEQ ID NO: 2 (nucleotide) and/or SEQ ID NO: 5 (amino acid):

(SEQ ID NO: 2)
ATGGATAAAAAGTATTCTATTGGTTTAGACATCGGCACTAATTCC
GTTGGATGGGCTGTCATAACCGATGAATACAAAGTACCTTCAAAG
AAATTTAAGGTGTTGGGGAACACAGACCGTCATTCGATTAAAAAG
AATCTTATCGGTGCCCTCCTATTCGATAGTGGCGAAACGGCAGAG
GCGACTCGCCTGAAACGAACCGCTCGGAGAAGGTATACACGTCGC
AAGAACCGAATATGTTACTTACAAGAAATTTTTAGCAATGAGATG
GCCAAAGTTGACGATTCTTTCTTTCACCGTTTGGAAGAGTCCTTC
CTTGTCGAAGAGGACAAGAAACATGAACGGCACCCCATCTTTGGA
AACATAGTAGATGAGGTGGCATATCATGAAAAGTACCCAACGATT
TATCACCTCAGAAAAAAGCTAGTTGACTCAACTGATAAAGCGGAC
CTGAGGTTAATCTACTTGGCTCTTGCCCATATGATAAAGTTCCGT
GGGCACTTTCTCATTGAGGGTGATCTAAATCCGGACAACTCGGAT
GTCGACAAACTGTTCATCCAGTTAGTACAAACCTATAATCAGTTG
TTTGAAGAGAACCCTATAAATGCAAGTGGCGTGGATGCGAAGGCT
ATTCTTAGCGCCCGCCTCTCTAAATCCCGACGGCTAGAAAACCTG
ATCGCACAATTACCCGGAGAGAAGAAAAATGGGTTGTTCGGTAAC
CTTATAGCGCTCTCACTAGGCCTGACACCAAATTTTAAGTCGAAC
TTCGACTTAGCTGAAGATGCCAAATTGCAGCTTAGTAAGGACACG
TACGATGACGATCTCGACAATCTACTGGCACAAATTGGAGATCAG
TATGCGGACTTATTTTTGGCTGCCAAAAACCTTAGCGATGCAATC
CTCCTATCTGACATACTGAGAGTTAATACTGAGATTACCAAGGCG
CCGTTATCCGCTTCAATGATCAAAAGGTACGATGAACATCACCAA
GACTTGACACTTCTCAAGGCCCTAGTCCGTCAGCAACTGCCTGAG
AAATATAAGGAAATATTCTTTGATCAGTCGAAAAACGGGTACGCA
GGTTATATTGACGGCGGAGCGAGTCAAGAGGAATTCTACAAGTTT
ATCAAACCCATATTAGAGAAGATGGATGGGACGGAAGAGTTGCTT
GTAAAACTCAATCGCGAAGATCTACTGCGAAAGCAGCGGACTTTC
GACAACGGTAGCATTCCACATCAAATCCACTTAGGCGAATTGCAT
GCTATACTTAGAAGGCAGGAGGATTTTTATCCGTTCCTCAAAGAC
AATCGTGAAAAGATTGAGAAAATCCTAACCTTTCGCATACCTTAC
TATGTGGGACCCCTGGCCCGAGGGAACTCTCGGTTCGCATGGATG
ACAAGAAAGTCCGAAGAAACGATTACTCCATGGAATTTTGAGGAA
GTTGTCGATAAAGGTGCGTCAGCTCAATCGTTCATCGAGAGGATG
ACCAACTTTGACAAGAATTTACCGAACGAAAAAGTATTGCCTAAG
CACAGTTTACTTTACGAGTATTTCACAGTGTACAATGAACTCACG
AAAGTTAAGTATGTCACTGAGGGCATGCGTAAACCCGCCTTTCTA
AGCGGAGAACAGAAGAAAGCAATAGTAGATCTGTTATTCAAGACC
AACCGCAAAGTGACAGTTAAGCAATTGAAAGAGGACTACTTTAAG
AAAATTGAATGCTTCGATTCTGTCGAGATCTCCGGGGTAGAAGAT
CGATTTAATGCGTCACTTGGTACGTATCATGACCTCCTAAAGATA
ATTAAAGATAAGGACTTCCTGGATAACGAAGAGAATGAAGATATC
TTAGAAGATATAGTGTTGACTCTTACCCTCTTTGAAGATCGGGAA
ATGATTGAGGAAAGACTAAAAACATACGCTCACCTGTTCGACGAT
AAGGTTATGAAACAGTTAAAGAGGCGTCGCTATACGGGCTGGGGA
CGATTGTCGCGGAAACTTATCAACGGGATAAGAGACAAGCAAAGT
GGTAAAACTATTCTCGATTTTCTAAAGAGCGACGGCTTCGCCAAT
AGGAACTTTATGCAGCTGATCCATGATGACTCTTTAACCTTCAAA
GAGGATATACAAAAGGCACAGGTTTCCGGACAAGGGGACTCATTG
CACGAACATATTGCGAATCTTGCTGGTTCGCCAGCCATCAAAAAG
GGCATACTCCAGACAGTCAAAGTAGTGGATGAGCTAGTTAAGGTC
ATGGGACGTCACAAACCGGAAAACATTGTAATCGAGATGGCACGC
GAAAATCAAACGACTCAGAAGGGGCAAAAAAACAGTCGAGAGCGG
ATGAAGAGAATAGAAGAGGGTATTAAAGAACTGGGCAGCCAGATC
TTAAAGGAGCATCCTGTGGAAAATACCCAATTGCAGAACGAGAAA
CTTTACCTCTATTACCTACAAAATGGAAGGGACATGTATGTTGAT
CAGGAACTGGACATAAACCGTTTATCTGATTACGACGTCGATCAC
ATTGTACCCCAATCCTTTTTGAAGGACGATTCAATCGACAATAAA
GTGCTTACACGCTCGGATAAGAACCGAGGGAAAAGTGACAATGTT
CCAAGCGAGGAAGTCGTAAAGAAAATGAAGAACTATTGGCGGCAG
CTCCTAAATGCGAAACTGATAACGCAAAGAAAGTTCGATAACTTA
ACTAAAGCTGAGAGGGGTGGCTTGTCTGAACTTGACAAGGCCGGA
TTTATTAAACGTCAGCTCGTGGAAACCCGCCAAATCACAAAGCAT
GTTGCACAGATACTAGATTCCCGAATGAATACGAAATACGACGAG
AACGATAAGCTGATTCGGGAAGTCAAAGTAATCACTTTAAAGTCA
AAATTGGTGTCGGACTTCAGAAAGGATTTTCAATTCTATAAAGTT
AGGGAGATAAATAACTACCACCATGCGCACGACGCTTATCTTAAT
GCCGTCGTAGGGACCGCACTCATTAAGAAATACCCGAAGCTAGAA
AGTGAGTTTGTGTATGGTGATTACAAAGTTTATGACGTCCGTAAG
ATGATCGCGAAAAGCGAACAGGAGATAGGCAAGGCTACAGCCAAA
TACTTCTTTTATTCTAACATTATGAATTTCTTTAAGACGGAAATC
ACTCTGGCAAACGGAGAGATACGCAAACGACCTTTAATTGAAACC
AATGGGGAGACAGGTGAAATCGTATGGGATAAGGGCCGGGACTTC
GCGACGGTGAGAAAAGTTTTGTCCATGCCCCAAGTCAACATAGTA
AAGAAAACTGAGGTGCAGACCGGAGGGTTTTCAAAGGAATCGATT
CTTCCAAAAAGGAATAGTGATAAGCTCATCGCTCGTAAAAAGGAC
TGGGACCCGAAAAAGTACGGTGGCTTCGATAGCCCTACAGTTGCC
TATTCTGTCCTAGTAGTGGCAAAAGTTGAGAAGGGAAAATCCAAG
AAACTGAAGTCAGTCAAAGAATTATTGGGGATAACGATTATGGAG
CGCTCGTCTTTTGAAAAGAACCCCATCGACTTCCTTGAGGCGAAA
GGTTACAAGGAAGTAAAAAAGGATCTCATAATTAAACTACCAAAG
TATAGTCTGTTTGAGTTAGAAAATGGCCGAAAACGGATGTTGGCT
AGCGCCGGAGAGCTTCAAAAGGGGAACGAACTCGCACTACCGTCT
AAATACGTGAATTTCCTGTATTTAGCGTCCCATTACGAGAAGTTG
AAAGGTTCACCTGAAGATAACGAACAGAAGCAACTTTTTGTTGAG
CAGCACAAACATTATCTCGACGAAATCATAGAGCAAATTTCGGAA
TTCAGTAAGAGAGTCATCCTAGCTGATGCCAATCTGGACAAAGTA
TTAAGCGCATACAACAAGCACAGGGATAAACCCATACGTGAGCAG
GCGGAAAATATTATCCATTTGTTTACTCTTACCAACCTCGGCGCT
CCAGCCGCATTCAAGTATTTTGACACAACGATAGATCGCAAACGA
TACACTTCTACCAAGGAGGTGCTAGACGCGACACTGATTCACCAA
TCCATCACGGGATTATATGAAACTCGGATAGATTTGTCACAGCTT
GGGGGTGACGGATCCCCCAAGAAGAAGAGGAAAGTCTCGAGCGAC
TACAAAGACCATGACGGTGATTATAAAGATCATGACATCGATTAC
AAGGATGACGATGACAAGGCTGCAGGA
(SEQ ID NO: 5)
MDKKYSIGLAIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITORKFDNL
TKAERGGLSELDKAGFIKRQLVETROITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD
(single underline: HNH domain;
double underline: RuvC domain)

In some embodiments, wild type Cas9 corresponds to Cas9 from Streptococcus pyogenes (NCBI Reference Sequence: NC_002737.2, SEQ ID NO: 3 (nucleotide); and Uniport Reference Sequence: Q99ZW2, SEQ ID NO: 6 (amino acid).

(SEQ ID NO: 3)
ATGGATAAGAAATACTCAATAGGCTTAGATATCGGCACAAATAGCGTCGGATGGGCGGT
GATCACTGATGAATATAAGGTTCCGTCTAAAAAGTTCAAGGTTCTGGGAAATACAGACC
GCCACAGTATCAAAAAAAATCTTATAGGGGCTCTTTTATTTGACAGTGGAGAGACAGCG
GAAGCGACTCGTCTCAAACGGACAGCTCGTAGAAGGTATACACGTCGGAAGAATCGTAT
TTGTTATCTACAGGAGATTTTTTCAAATGAGATGGCGAAAGTAGATGATAGTTTCTTTCA
TCGACTTGAAGAGTCTTTTTTGGTGGAAGAAGACAAGAAGCATGAACGTCATCCTATTTT
TGGAAATATAGTAGATGAAGTTGCTTATCATGAGAAATATCCAACTATCTATCATCTGCG
AAAAAAATTGGTAGATTCTACTGATAAAGCGGATTTGCGCTTAATCTATTTGGCCTTAGC
GCATATGATTAAGTTTCGTGGTCATTTTTTGATTGAGGGAGATTTAAATCCTGATAATAGT
GATGTGGACAAACTATTTATCCAGTTGGTACAAACCTACAATCAATTATTTGAAGAAAAC
CCTATTAACGCAAGTGGAGTAGATGCTAAAGCGATTCTTTCTGCACGATTGAGTAAATCA
AGACGATTAGAAAATCTCATTGCTCAGCTCCCCGGTGAGAAGAAAAATGGCTTATTTGG
GAATCTCATTGCTTTGTCATTGGGTTTGACCCCTAATTTTAAATCAAATTTTGATTTGGCA
GAAGATGCTAAATTACAGCTTTCAAAAGATACTTACGATGATGATTTAGATAATTTATTG
GCGCAAATTGGAGATCAATATGCTGATTTGTTTTTGGCAGCTAAGAATTTATCAGATGCT
ATTTTACTTTCAGATATCCTAAGAGTAAATACTGAAATAACTAAGGCTCCCCTATCAGCT
TCAATGATTAAACGCTACGATGAACATCATCAAGACTTGACTCTTTTAAAAGCTTTAGTT
CGACAACAACTTCCAGAAAAGTATAAAGAAATCTTTTTTGATCAATCAAAAAACGGATA
TGCAGGTTATATTGATGGGGGAGCTAGCCAAGAAGAATTTTATAAATTTATCAAACCAAT
TTTAGAAAAAATGGATGGTACTGAGGAATTATTGGTGAAACTAAATCGTGAAGATTTGCT
GCGCAAGCAACGGACCTTTGACAACGGCTCTATTCCCCATCAAATTCACTTGGGTGAGCT
GCATGCTATTTTGAGAAGACAAGAAGACTTTTATCCATTTTTAAAAGACAATCGTGAGAA
GATTGAAAAAATCTTGACTTTTCGAATTCCTTATTATGTTGGTCCATTGGCGCGTGGCAAT
AGTCGTTTTGCATGGATGACTCGGAAGTCTGAAGAAACAATTACCCCATGGAATTTTGAA
GAAGTTGTCGATAAAGGTGCTTCAGCTCAATCATTTATTGAACGCATGACAAACTTTGAT
AAAAATCTTCCAAATGAAAAAGTACTACCAAAACATAGTTTGCTTTATGAGTATTTTACG
GTTTATAACGAATTGACAAAGGTCAAATATGTTACTGAAGGAATGCGAAAACCAGCATT
TCTTTCAGGTGAACAGAAGAAAGCCATTGTTGATTTACTCTTCAAAACAAATCGAAAAGT
AACCGTTAAGCAATTAAAAGAAGATTATTTCAAAAAAATAGAATGTTTTGATAGTGTTGA
AATTTCAGGAGTTGAAGATAGATTTAATGCTTCATTAGGTACCTACCATGATTTGCTAAA
AATTATTAAAGATAAAGATTTTTTGGATAATGAAGAAAATGAAGATATCTTAGAGGATA
TTGTTTTAACATTGACCTTATTTGAAGATAGGGAGATGATTGAGGAAAGACTTAAAACAT
ATGCTCACCTCTTTGATGATAAGGTGATGAAACAGCTTAAACGTCGCCGTTATACTGGTT
GGGGACGTTTGTCTCGAAAATTGATTAATGGTATTAGGGATAAGCAATCTGGCAAAACA
ATATTAGATTTTTTGAAATCAGATGGTTTTGCCAATCGCAATTTTATGCAGCTGATCCATG
ATGATAGTTTGACATTTAAAGAAGACATTCAAAAAGCACAAGTGTCTGGACAAGGCGAT
AGTTTACATGAACATATTGCAAATTTAGCTGGTAGCCCTGCTATTAAAAAAGGTATTTTA
CAGACTGTAAAAGTTGTTGATGAATTGGTCAAAGTAATGGGGCGGCATAAGCCAGAAAA
TATCGTTATTGAAATGGCACGTGAAAATCAGACAACTCAAAAGGGCCAGAAAAATTCGC
GAGAGCGTATGAAACGAATCGAAGAAGGTATCAAAGAATTAGGAAGTCAGATTCTTAAA
GAGCATCCTGTTGAAAATACTCAATTGCAAAATGAAAAGCTCTATCTCTATTATCTCCAA
AATGGAAGAGACATGTATGTGGACCAAGAATTAGATATTAATCGTTTAAGTGATTATGAT
GTCGATCACATTGTTCCACAAAGTTTCCTTAAAGACGATTCAATAGACAATAAGGTCTTA
ACGCGTTCTGATAAAAATCGTGGTAAATCGGATAACGTTCCAAGTGAAGAAGTAGTCAA
AAAGATGAAAAACTATTGGAGACAACTTCTAAACGCCAAGTTAATCACTCAACGTAAGT
TTGATAATTTAACGAAAGCTGAACGTGGAGGTTTGAGTGAACTTGATAAAGCTGGTTTTA
TCAAACGCCAATTGGTTGAAACTCGCCAAATCACTAAGCATGTGGCACAAATTTTGGATA
GTCGCATGAATACTAAATACGATGAAAATGATAAACTTATTCGAGAGGTTAAAGTGATT
ACCTTAAAATCTAAATTAGTTTCTGACTTCCGAAAAGATTTCCAATTCTATAAAGTACGT
GAGATTAACAATTACCATCATGCCCATGATGCGTATCTAAATGCCGTCGTTGGAACTGCT
TTGATTAAGAAATATCCAAAACTTGAATCGGAGTTTGTCTATGGTGATTATAAAGTTTAT
GATGTTCGTAAAATGATTGCTAAGTCTGAGCAAGAAATAGGCAAAGCAACCGCAAAATA
TTTCTTTTACTCTAATATCATGAACTTCTTCAAAACAGAAATTACACTTGCAAATGGAGA
GATTCGCAAACGCCCTCTAATCGAAACTAATGGGGAAACTGGAGAAATTGTCTGGGATA
AAGGGCGAGATTTTGCCACAGTGCGCAAAGTATTGTCCATGCCCCAAGTCAATATTGTCA
AGAAAACAGAAGTACAGACAGGCGGATTCTCCAAGGAGTCAATTTTACCAAAAAGAAAT
TCGGACAAGCTTATTGCTCGTAAAAAAGACTGGGATCCAAAAAAATATGGTGGTTTTGAT
AGTCCAACGGTAGCTTATTCAGTCCTAGTGGTTGCTAAGGTGGAAAAAGGGAAATCGAA
GAAGTTAAAATCCGTTAAAGAGTTACTAGGGATCACAATTATGGAAAGAAGTTCCTTTG
AAAAAAATCCGATTGACTTTTTAGAAGCTAAAGGATATAAGGAAGTTAAAAAAGACTTA
ATCATTAAACTACCTAAATATAGTCTTTTTGAGTTAGAAAACGGTCGTAAACGGATGCTG
GCTAGTGCCGGAGAATTACAAAAAGGAAATGAGCTGGCTCTGCCAAGCAAATATGTGAA
TTTTTTATATTTAGCTAGTCATTATGAAAAGTTGAAGGGTAGTCCAGAAGATAACGAACA
AAAACAATTGTTTGTGGAGCAGCATAAGCATTATTTAGATGAGATTATTGAGCAAATCAG
TGAATTTTCTAAGCGTGTTATTTTAGCAGATGCCAATTTAGATAAAGTTCTTAGTGCATAT
AACAAACATAGAGACAAACCAATACGTGAACAAGCAGAAAATATTATTCATTTATTTAC
GTTGACGAATCTTGGAGCTCCCGCTGCTTTTAAATATTTTGATACAACAATTGATCGTAA
ACGATATACGTCTACAAAAGAAGTTTTAGATGCCACTCTTATCCATCAATCCATCACTGG
TCTTTATGAAACACGCATTGATTTGAGTCAGCTAGGAGGTGACTGA
(SEQ ID NO: 6)
MDKKYSIGLDIGTNSVGWAVITDEYKVPSKKFKVLGNTDRHSIKKNLIGALLEDSGETAEAT
RLKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGD

(single underline: HNH domain; double underline: RuvC domain)

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria meningitidis (NCBI Ref: YP_002342100.1) or to a Cas9 from any other organism.

In some embodiments, dCas9 corresponds to, or comprises in part or in whole, a Cas9 amino acid sequence having one or more mutations that inactivate the Cas9 nuclease activity. For example, in some embodiments, a dCas9 domain comprises D10A and an H840A mutation of SEQ ID NO: 6 or corresponding mutations in another Cas9. In some embodiments, the dCas9 comprises the amino acid sequence of SEQ ID NO: 7 dCas9 (D10A and H840A):

(SEQ ID NO: 7)
MDKKYSIGLAIGTNSVGWAVITDEYKVPSKKFKVLGNTDRHSIKKNLIGALLEDSGETAEAT
RLKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQ
NGRDMYVDQELDINRLSDYDVDAIVPQSFLKDDSIDNKVLTRSDKNRGKSDNVPSEEVVKK
MKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGD

(single underline: HNH domain; double underline: RuvC domain).

In some embodiments, the Cas9 domain comprises a D10A mutation, while the residue at position 840 remains a histidine in the amino acid sequence provided in SEQ ID NO: 6, or at corresponding positions in another Cas9, such as a Cas9 set forth in any of the amino acid sequences provided in SEQ ID NOs: 4-26. Without wishing to be bound by any particular theory, the presence of the catalytic residue H840 maintains the activity of the Cas9 to cleave the non-edited (e.g., non-deaminated) strand containing a T opposite the targeted A. Restoration of H840 (e.g., from A840 of a dCas9) does not result in the cleavage of the target strand containing the A. Such Cas9 variants are able to generate a single-strand DNA break (nick) at a specific location based on the gRNA-defined target sequence, leading to repair of the non-edited strand, ultimately resulting in a T to C change on the non-edited strand.

In other embodiments, dCas9 variants having mutations other than D10A and H840A are provided, which, e.g., result in nuclease inactivated Cas9 (dCas9). Such mutations, by way of example, include other amino acid substitutions at D10 and H840, or other substitutions within the nuclease domains of Cas9 (e.g., substitutions in the HNH nuclease subdomain and/or the RuvC1 subdomain). In some embodiments, variants or homologues of dCas9 (e.g., variants of SEQ ID NO: 6, 7, 8, 9, or 22) are provided which are at least about 70% identical, at least about 80% identical, at least about 90% identical, at least about 95% identical, at least about 98% identical, at least about 99% identical, at least about 99.5% identical, or at least about 99.9% identical to SEQ ID NO: 6, 7, 8, 9, or 22. In some embodiments, variants of dCas9 (e.g., variants of SEQ ID NO: 6, 7, 8, 9, or 22) are provided having amino acid sequences which are shorter, or longer than SEQ ID NO: 7, 8, 9, or 22, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, Cas9 fusion proteins as provided herein comprise the full-length amino acid sequence of a Cas9 protein, e.g., one of the Cas9 sequences provided herein. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or sgRNA, but does not comprise a functional nuclease domain, e.g., in that it comprises only a truncated version of a nuclease domain or no nuclease domain at all.

Exemplary amino acid sequences of suitable Cas9 domains and Cas9 fragments are provided herein, and additional suitable sequences of Cas9 domains and fragments will be apparent to those of skill in the art.

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1).

It should be appreciated that additional Cas9 proteins (e.g., a nuclease dead Cas9 (dCas9), a Cas9 nickase (nCas9), or a nuclease active Cas9), including variants and homologs thereof, are within the scope of this disclosure. Exemplary Cas9 proteins include, without limitation, those provided below. In some embodiments, the Cas9 protein is a nuclease dead Cas9 (dCas9). In some embodiments, the dCas9 comprises the amino acid sequence (SEQ ID NO: 7, 8, 9, or 22). In some embodiments, the Cas9 protein is a Cas9 nickase (nCas9). In some embodiments, the nCas9 comprises the amino acid sequence (SEQ ID NO: 10, 13, 16, or 21). In some embodiments, the Cas9 protein is a nuclease active Cas9. In some embodiments, the nuclease active Cas9 comprises the amino acid sequence (SEQ ID NO: 4, 5,6, 11, 12, 14, 15, 16, 17, 18, 19, 20, 23, 24, 25, or 26).

Exemplary catalytically inactive Cas9 (dCas9):

(SEQ ID NO: 8)
DKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPL
SASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQN
GRDMYVDQELDINRLSDYDVDAIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNT
KYDENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD

Exemplary Cas9 nickase (nCas9):

(SEQ ID NO: 10)
DKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQ
TYNQLFEENPINSGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPL
SASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNT
KYDENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD

Exemplary catalytically active Cas9:

(SEQ ID NO: 11)
DKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLF
EDREMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAVVGTA
LIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGD.

The term “Cas9 nickase,” as used herein, refers to a Cas9 protein that is capable of cleaving only one strand of a duplexed nucleic acid molecule (e.g., a duplexed DNA molecule). In some embodiments, a Cas9 nickase comprises a D10A mutation and has a histidine at position H840 of SEQ ID NO: 6, or a corresponding mutation in any Cas9 provided, such as any one of SEQ ID NOs: 4-26. For example, a Cas9 nickase may comprise the amino acid sequence as set forth in SEQ ID NO: 10, 13, 16, or 21. Such a Cas9 nickase has an active HNH nuclease domain and is able to cleave the non-targeted strand of DNA, i.e., the strand bound by the gRNA. Further, such a Cas9 nickase has an inactive RuvC nuclease domain and is not able to cleave the targeted strand of the DNA, i.e., the strand where base editing is desired.

In some embodiments, Cas9 refers to a Cas9 from archaea (e.g. nanoarchaea), which constitute a domain and kingdom of single-celled prokaryotic microbes. In some embodiments, Cas9 refers to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure.

In some embodiments, the nucleic acid programmable DNA binding protein (napDNAbp) of any of the fusion proteins provided herein may be a CasX or CasY protein.

In some embodiments, the napDNAbp is a CasX protein. In some embodiments, the CasX protein is a nuclease inactive CasX protein (dCasX), a CasX nickase (CasXn), or a nuclease active CasX. In some embodiments, the napDNAbp is a CasY protein. In some embodiments, the CasY protein is a nuclease inactive CasY protein (dCasY), a CasY nickase (CasYn), or a nuclease active CasY. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of SEQ ID NOs: 27-29. In some embodiments, the napDNAbp comprises an amino acid sequence of any one SEQ ID NOs: 27-29. It should be appreciated that CasX and CasY from other bacterial species may also be used in accordance with the present disclosure.

CasX (uniprot.org/uniprot/F0NN87; http://www.uniprot.org/uniprot/F0NH53)
>tr|F0NN87|F0NN87_SULIH CRISPR-associated Casx protein OS = Sulfolobus
islandicus (strain HVE10/4) GN = SiH_0402 PE = 4 SV = 1
(SEQ ID NO: 27)
MEVPLYNIFGDNYIIQVATEAENSTIYNNKVEIDDEELRNVLNLAYKIAKNNEDAAAERRGK
AKKKKGEEGETTTSNIILPLSGNDKNPWTETLKCYNFPTTVALSEVFKNFSQVKECEEVSAPS
FVKPEFYEFGRSPGMVERTRRVKLEVEPHYLIIAAAGWVLTRLGKAKVSEGDYVGVNVFTPT
RGILYSLIQNVNGIVPGIKPETAFGLWIARKVVSSVTNPNVSVVRIYTISDAVGQNPTTINGGFS
IDLTKLLEKRYLLSERLEAIARNALSISSNMRERYIVLANYIYEYLTGSKRLEDLLYFANRDLI
MNLNSDDGKVRDLKLISAYVNGELIRGEG
>tr|F0NH53|F0NH53_SULIR CRISPR associated protein, CasX OS = Sulfolobus
islandicus (strain REY15A) GN = SiRe_0771 PE = 4 SV = 1
(SEQ ID NO: 28)
MEVPLYNIFGDNYIIQVATEAENSTIYNNKVEIDDEELRNVLNLAYKIAKNNEDAAAERRGK
AKKKKGEEGETTTSNIILPLSGNDKNPWTETLKCYNFPTTVALSEVFKNFSQVKECEEVSAPS
FVKPEFYKFGRSPGMVERTRRVKLEVEPHYLIMAAAGWVLTRLGKAKVSEGDYVGVNVFTP
TRGILYSLIQNVNGIVPGIKPETAFGLWIARKVVSSVTNPNVSVVSIYTISDAVGQNPTTINGGF
SIDLTKLLEKRDLLSERLEAIARNALSISSNMRERYIVLANYIYEYLTGSKRLEDLLYFANRDLI
MNLNSDDGKVRDLKLISAYVNGELIRGEG
CasY (ncbi.nlm.nih.gov/protein/APG80656.1)
>APG80656.1 CRISPR-associated protein CasY [uncultured Parcubacteria group
bacterium]
(SEQ ID NO: 29)
MSKRHPRISGVKGYRLHAQRLEYTGKSGAMRTIKYPLYSSPSGGRTVPREIVSAINDDYVGL
YGLSNFDDLYNAEKRNEEKVYSVLDFWYDCVQYGAVESYTAPGLLKNVAEVRGGSYELTK
TLKGSHLYDELQIDKVIKFLNKKEISRANGSLDKLKKDIIDCFKAEYRERHKDQCNKLADDIK
NAKKDAGASLGERQKKLFRDFFGISEQSENDKPSFTNPLNLTCCLLPFDTVNNNRNRGEVLF
NKLKEYAQKLDKNEGSLEMWEYIGIGNSGTAFSNFLGEGFLGRLRENKITELKKAMMDITDA
WRGQEQEEELEKRLRILAALTIKLREPKFDNHWGGYRSDINGKLSSWLQNYINQTVKIKEDL
KGHKKDLKKAKEMINRFGESDTKEEAVVSSLLESIEKIVPDDSADDEKPDIPAIAIYRRFLSDG
RLTLNRFVQREDVQEALIKERLEAEKKKKPKKRKKKSDAEDEKETIDFKELFPHLAKPLKLVP
NFYGDSKRELYKKYKNAAIYTDALWKAVEKIYKSAFSSSLKNSFFDTDFDKDFFIKRLQKIFS
VYRRFNTDKWKPIVKNSFAPYCDIVSLAENEVLYKPKQSRSRKSAAIDKNRVRLPSTENIAKA
GIALARELSVAGFDWKDLLKKEEHEEYIDLIELHKTALALLLAVTETQLDISALDFVENGTVK
DFMKTRDGNLVLEGRFLEMFSQSIVFSELRGLAGLMSRKEFITRSAIQTMNGKQAELLYIPHE
FQSAKITTPKEMSRAFLDLAPAEFATSLEPESLSEKSLLKLKQMRYYPHYFGYELTRTGQGID
GGVAENALRLEKSPVKKREIKCKQYKTLGRGQNKIVLYVRSSYYQTQFLEWFLHRPKNVQT
DVAVSGSFLIDEKKVKTRWNYDALTVALEPVSGSERVFVSQPFTIFPEKSAEEEGQRYLGIDIG
EYGIAYTALEITGDSAKILDQNFISDPQLKTLREEVKGLKLDQRRGTFAMPSTKIARIRESLVH
SLRNRIHHLALKHKAKIVYELEVSRFEEGKQKIKKVYATLKKADVYSEIDADKNLQTTVWG
KLAVASEISASYTSQFCGACKKLWRAEMQVDETITTQELIGTVRVIKGGTLIDAIKDFMRPPIF
DENDTPFPKYRDFCDKHHISKKMRGNSCLFICPFCRANADADIQASQTIALLRYVKEEKKVED
YFERFRKLKNIKVLGQMKKI

The term “effective amount,” as used herein, refers to an amount of a biologically active agent that is sufficient to elicit a desired biological response. For example, in some embodiments, an effective amount of a nucleobase editor may refer to the amount of the nucleobase editor that is sufficient to induce a mutation of a target site specifically bound by the nucleobase editor. In some embodiments, an effective amount of a fusion protein provided herein, e.g., of a fusion protein comprising a nucleic acid programmable DNA binding protein and a deaminase domain (e.g., a cytidine deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a nucleobase editor, a deaminase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “proliferative disease,” as used herein, refers to any disease in which cell or tissue homeostasis is disturbed in that a cell or cell population exhibits an abnormally elevated proliferation rate. Proliferative diseases include hyperproliferative diseases, such as pre-neoplastic hyperplastic conditions and neoplastic diseases. Neoplastic diseases are characterized by an abnormal proliferation of cells and include both benign and malignant neoplasias. Malignant neoplasia is also referred to as cancer.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. As used herein, the term “fusion protein” may be synonymous with the term “base editor”. In exemplary embodiments, the fusion proteins of the disclosure are base editing fusion proteins, or base editors. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “RNA-programmable nuclease,” and “RNA-guided nuclease” are used interchangeably herein and refer to a nuclease that forms a complex with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), the entire contents of which is incorporated herein by reference. Other examples of gRNAs (e.g., those including domain 2) can be found in International Publication No. WO 2015/035,139, published Mar. 12, 2015, entitled “Switchable Cas9 Nucleases And Uses Thereof,” and International Publication No. WO 2015/035136, published Mar. 12, 2015, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csn1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference.

Because RNA-programmable nucleases (e.g., Cas9) use RNA:DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al., Multiplex genome engineering using CRISPR/Cas systems. Science 339, 819-823 (2013); Mali, P. et al., RNA-guided human genome engineering via Cas9. Science 339, 823-826 (2013); Hwang, W. Y. et al., Efficient genome editing in zebrafish using a CRISPR-Cas system. Nature biotechnology 31, 227-229 (2013); Jinek, M. et al., RNA-programmed genome editing in human cells. eLife 2, e00471 (2013); Dicarlo, J. E. et al., Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

A “nuclear localization signal or sequence” (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences may be of any size and composition, for example, more than 25, 25, 15, 12, 10, 8, 7, 6, 5, or 4 amino acids, but will preferably comprise at least a four to eight amino acid sequence known to function as a nuclear localization signal (NLS).

The term “host cell,” as used herein, refers to a cell that can host and replicate a vector encoding a base editor, guide RNA, and/or combination thereof, as described herein. In some embodiments, host cells are mammalian cells, such as human cells. Provided herein are methods of transducing and transfecting a host cell, such as a human cell, e.g., a human cell in a subject, with one or more vectors provided herein, such as one or more viral (e.g., rAAV) vectors provided herein.

It should be appreciated that any of the base editors, guide RNAs, and or combinations thereof, described herein may be introduced into a host cell in any suitable way, either stably or transiently. In some embodiments, a base editor may be transfected into the host cell. In some embodiments, the host cell may be transduced or transfected with a nucleic acid construct that encodes a base editor. For example, a host cell may be transduced (e.g., with a viral particle encoding a base editor) with a nucleic acid that encodes a base editor, or the translated base editor. As an additional example, a host cell may be transfected with a nucleic acid (e.g., a plasmid) that encodes a base editor or the translated base editor. Such transductions or transfections may be stable or transient. In some embodiments, host cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into host cells through electroporation, transient transfection (e.g., lipofection, such as with Lipofectamine 3000®), stable genome integration (e.g., piggybac), viral transduction, or other methods known to those of skill in the art.

Also provided herein are host cells for packaging of viral particles. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. In some embodiments, the host cell is a eukaryotic cell, for example, a yeast cell, an insect cell, or a mammalian cell. The type of host cell, will, of course, depend on the vector employed, and suitable host cell/vector combinations will be readily apparent to those of skill in the art.

As used herein, the term “intein” refers to auto-processing polypeptide domains found in organisms from all domains of life. An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. This process is also known as cis-protein splicing, as opposed to the natural process of trans-protein splicing with “split inteins.”

Split inteins are a sub-category of inteins. Unlike the more common contiguous inteins, split inteins are transcribed and translated as two separate polypeptides, the N-intein and C-intein, each fused to one extein. Upon translation, the intein fragments spontaneously and non-covalently assemble into the canonical intein structure to carry out protein splicing in trans.

Inteins and split inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al., EMBO J. 15(19):5146-5153 (1996)).

As used herein, the term “protein splicing” refers to a process in which an interior region of a precursor protein (an intein) is excised and the flanking regions of the protein (exteins) are ligated to form the mature protein. This natural process has been observed in numerous proteins from both prokaryotes and eukaryotes (Perler, F. B., Xu, M. Q., Paulus, H. Current Opinion in Chemical Biology 1997, 1, 292-299; Perler, F. B. Nucleic Acids Research 1999, 27, 346-347). The intein unit contains the necessary components needed to catalyze protein splicing and often contains an endonuclease domain that participates in intein mobility (Perler, F. B., Davis, E. O., Dean, G. E., Gimble, F. S., Jack, W. E., Neff, N., Noren, C. J., Thomer, J., Belfort, M. Nucleic Acids Research 1994, 22, 1127-1127). The resulting proteins are linked, however, not expressed as separate proteins. Protein splicing may also be conducted in trans with split inteins expressed on separate polypeptides spontaneously combine to form a single intein which then undergoes the protein splicing process to join to separate proteins.

The elucidation of the mechanism of protein splicing has led to a number of intein-based applications (Comb, et al., U.S. Pat. No. 5,496,714; Comb, et al., U.S. Pat. No. 5,834,247; Camarero and Muir, J. Amer. Chem. Soc., 121:5597-5598 (1999); Chong, et al., Gene, 192:271-281 (1997), Chong, et al., Nucleic Acids Res., 26:5109-5115 (1998); Chong, et al., J. Biol. Chem., 273:10567-10577 (1998); Cotton, et al. J. Am. Chem. Soc., 121:1100-1101 (1999); Evans, et al., J. Biol. Chem., 274:18359-18363 (1999); Evans, et al., J. Biol. Chem., 274:3923-3926 (1999); Evans, et al., Protein Sci., 7:2256-2264 (1998); Evans, et al., J. Biol. Chem., 275:9091-9094 (2000); Iwai and Pluckthun, FEBS Lett. 459:166-172 (1999); Mathys, et al., Gene, 231:1-13 (1999); Mills, et al., Proc. Natl. Acad. Sci. USA 95:3543-3548 (1998); Muir, et al., Proc. Natl. Acad. Sci. USA 95:6705-6710 (1998); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999); Severinov and Muir, J. Biol. Chem., 273:16205-16209 (1998); Shingledecker, et al., Gene, 207:187-195 (1998); Southworth, et al., EMBO J. 17:918-926 (1998); Southworth, et al., Biotechniques, 27:110-120 (1999); Wood, et al., Nat. Biotechnol., 17:889-892 (1999); Wu, et al., Proc. Natl. Acad. Sci. USA 95:9226-9231 (1998a); Wu, et al., Biochim Biophys Acta 1387:422-432 (1998b); Xu, et al., Proc. Natl. Acad. Sci. USA 96:388-393 (1999); Yamazaki, et al., J. Am. Chem. Soc., 120:5591-5592 (1998)). Each reference is incorporated herein by reference.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, cattle, a cat, or a dog. In some embodiments, the subject is a vertebrate, an amphibian, a reptile, a fish, an insect, a fly, or a nematode. In some embodiments, the subject is a research or experimental animal. In some embodiments, the subject is genetically engineered, e.g., a genetically engineered non-human subject. The subject may be of either sex and at any stage of development. In some embodiments, the subject is a domesticated animal. In some embodiments, the subject is a plant.

The term “target site” refers to a sequence within a nucleic acid molecule that is modified by a base editor, such as a fusion protein comprising a cytidine deaminase, (e.g., a dCas9-cytidine deaminase fusion protein provided herein).

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g. deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

The term “off-target editing frequency,” as used herein, refers to the number or proportion of unintended base pairs, e.g. DNA base pairs, that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Because the DNA target sequence and the Cas9-independent off-target sequences are known a priori in the methods disclosed herein, nucleic acid primers with sufficient complementarity to regions upstream or downstream of the target sequence and Cas9-independent off-target sequences of interest may be designed using techniques known in the art, such as the PhusionU PCR kit (Life Technologies), Phusion HS II kit (Life Technologies), and Illumina MiSeq kit. The number of off-target DNA edits may be measured by techniques known in the art, including high-throughput screening of sequencing reads, EndoV-Seq, GUIDE-Seq, CIRCLE-Seq, and Cas-OFFinder. Since many of the Cas9-dependent off-target sites have high sequence identity to the target site of interest, nucleic acid primers with sufficient complementarity to regions upstream or downstream of the Cas9-dependent off-target site may likewise be designed using techniques and kits known in the art. These kits make use of polymerase chain reaction (PCR) amplification, which produces amplicons as intermediate products. The target and off-target sequences may comprise genomic loci that further comprise protospacers and PAMs. Accordingly, the term “amplicons,” as used herein, may refer to nucleic acid molecules that constitute the aggregates of genomic loci, protospacers and PAMs. High-throughput sequencing techniques used herein may further include Sanger sequencing and Illumina-based next-generation genome sequencing (NGS).

The term “on-target editing,” as used herein, refers to the introduction of intended modifications (e.g., deaminations) to a nucleotide (e.g., cytosine) in a target sequence, such as using the base editors described herein. The term “off-target DNA editing,” as used herein, refers to the introduction of unintended modifications (e.g. deaminations) to nucleotides (e.g. cytosine) in a sequence outside the canonical base editor binding window (i.e., from one protospacer position to another, typically 2 to 8 nucleotides long). Off-target DNA editing can result from weak or non-specific binding of the gRNA sequence to the target sequence. As used herein, the term “bystander editing” refers to synonymous off-target point mutations at nucleobases that are near (proximate to) the target base and do not change the outcome of the intended editing method.

As used herein, the terms “purity” and “product purity” of a base editor refer to the percentage of edited sequencing reads (reads in which the target nucleobase has been converted to a different base) in which the intended conversion occurs (e.g., for a cytosine to guanine base editor, in which the target C is edited to a G). See Komor et al., Sci Adv 3 (2017).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

As used herein, the term “variant” refers to a protein having characteristics that deviate from what occurs in nature that retains at least one functional, i.e., binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild-type deaminase amino acid sequence, e.g., following ancestral sequence reconstruction of the deaminase. These changes include chemical modifications, including substitutions of different amino acid residues truncations, covalent additions (e.g., of a tag), and any other mutations. The term also encompasses circular permutants, mutants, truncations, or domains of a reference sequence, and which display the same or substantially the same functional activity or activities as the reference sequence. This term also embraces fragments of a wild-type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein.

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

The term “vector,” as used herein, refers to a nucleic acid that can be modified to encode a gene of interest and that is able to enter into a host cell and replicate within the host cell, and then transfer a replicated form of the vector into another host cell. Exemplary suitable vectors include viral vectors, such as AAV vectors or bacteriophages and filamentous phage, and conjugative plasmids. Additional suitable vectors will be apparent to those of skill in the art based on the present disclosure.

### DETAILED DESCRIPTION OF INVENTION

The present disclosure provides for cytosine-to-guanine or “CGBE” (or guanine-to-cytosine or “GCBE”) transversion base editors which comprise a napDNAbp, or more specifically, a napDNAbp (e.g., a dCas9 domain), fused to a nucleobase modification domain and a polymerase domain. The disclosed GGBE base editors are capable of converting a C:G nucleobase pair to a G:C nucleobase pair in a target nucleotide sequence of interest, e.g., a genome of a cell. The disclosed base editors may catalyze the conversion of a target cytosine to a guanine via an excision of the target cytosine nucleobase, which generates an abasic site.

In addition, the disclosure provides compositions comprising the GGBE base editors as described herein, e.g., fusion proteins comprising a napDNAbp domain, a cytidine deaminase domain, and multiple uracil binding protein (UBP) domains; and one or more guide RNAs, e.g., a single-guide RNA (“sgRNA”). In addition, the instant specification provides for nucleic acid molecules encoding and/or expressing the GGBE base editors as described herein, as well as expression vectors and constructs for expressing the GGBE base editors described herein and/or a gRNA, host cells comprising said nucleic acid molecules and expression vectors and optionally vectors encoding one or more gRNAs, host cells comprising said GGBE base editors and optionally one or more gRNAs, and methods for delivering and/or administering nucleic acid-based embodiments described herein.

Accordingly, in some embodiments, the disclosure provides fusion proteins that comprise (i) a nucleic acid programmable DNA binding protein (napDNAbp), (ii) a cytidine deaminase domain, (iii) a first uracil binding protein (UBP) domain, and (iv) a DNA repair protein. In some embodiments, the DNA repair protein is selected from a DNA polymerase, an exonuclease, an RNA binding motif protein, an E3 ligase, and a translesion polymerase.

In some embodiments, the DNA repair protein is a nucleic acid polymerase, such as a DNA polymerase (e.g., a translesion polymerase). In various embodiments, the DNA repair protein is selected from DNA polymerase D1 (POLD1), DNA polymerase D2 (POLD2), and DNA polymerase D3 (POLD3). In some embodiments, the fusion protein comprises (iv) a nucleic acid polymerase domain (NAP).

In some embodiments, the DNA repair protein is an RNA binding motif protein, such as RNA binding motif protein, X-linked (RBMX). In some embodiments, the DNA repair protein is an exonuclease, such as exonuclease 1 (EX01). In some embodiments, the DNA repair protein is an E3 ligase, such as RAD18 or RFWD3.

In some embodiments, the DNA repair protein is a protein encoded by a gene selected from DDX1, EXO1, POLD1, POLD2, POLD3, RAD18, RBMX, REV1, RFWD3, TIMELESS, PCNA, POLH, POLK, UBE2I, and UBE2T. In particular embodiments, the DNA repair protein is one of POLD2, RBMX, and EXO1.

The first UBP domain of any of the disclosed fusion proteins may be a UNG orthologue from Mycobacterium smegmatis (UdgX) protein, or a variant thereof. In some embodiments, the first UBP domain has an amino acid sequence that is at least 80%, 85%, 90%, 95%, 98%, or 99% identical to the amino acid sequence of SEQ ID NO: 49, or has an amino acid sequence identical to SEQ ID NO: 49. In some embodiments, the first UBP domain comprises the amino acid sequence of SEQ ID NO: 50 (UdgX*).

In some embodiments, these disclosed CGBEs further comprise a second DNA repair protein. The second DNA repair protein may be selected from POLD2, RBMX, and EX01. In some embodiments, the first DNA repair protein is a POLD2, and the second DNA repair protein is an RBMX.

In some aspects, the disclosed CGBE fusion proteins may comprise (i) a nucleic acid programmable DNA binding protein (napDNAbp) domain, (ii) a cytidine deaminase domain, (iii) a first UBP domain, and (iv) a second UBP domain. These fusion proteins may further comprise a third UBP domain. In various embodiments, at least one of the first, second, and third UBP domains is a UdgX protein, or a variant thereof. In some embodiments, each of the first and second, and/or third, UBP domain is a UdgX protein. In some embodiments, any of the first, second, and third UBP domains has an amino acid sequence that is at least 80%, 85%, 90%, 95%, 98%, or 99% identical to the amino acid sequence of SEQ ID NO: 49, or has an amino acid sequence identical to SEQ ID NO: 49. In some aspects, the disclosed CGBE fusion proteins comprise (i) a napDNAbp domain, (ii) a cytidine deaminase domain, (iii) a first UBP domain, (iv) a second UBP domain, and (v) a DNA repair protein.

The cytidine deaminase domain of any of the disclosed CGBEs may be selected from an APOBEC family deaminase, or a variant thereof. For instance, the deaminase may comprise rAPOBEC1 or a variant thereof (e.g., the EE double mutant variant of rAPOBEC1 or the ancestrally reconstructed rAPOBEC1 variant, Anc689); or human APOBEC3A or a variant thereof (e.g., evolved human APOBEC3A-T31A (eA3aA-T31A)). In some embodiments, the napDNAbp domain is a Cas9 domain, such as a S. pyogenes Cas9 nickase (SpCas9n) domain. In some embodiments, the napDNAbp domain is a high fidelity SpCas9 nickase, such as HF-nCas9 or HF-nCas9-NG.

In particular embodiments, the CGBEs the fusion protein comprises the structure:
[UdgX]-[Anc689 deaminase]-[UdgX]-[nCas9 domain];
[UdgX]-[Anc689 deaminase]-[UdgX]-[nCas9 domain]-[RBMX];
[UdgX]-[EE deaminase]-[UdgX]-[nCas9 domain]-[UdgX];
[UdgX]-[rAPOBEC1 deaminase]-[UdgX]-[HF-nCas9 domain];
[UdgX]-[rAPOBEC1 deaminase]-[UdgX]-[HF-nCas9 domain]-[UdgX];
[RBMX]-[eA3A deaminase]-[UdgX]-[nCas9 domain];
[RBMX]-[eA3A deaminase]-[UdgX]-[HF-nCas9 domain];
[POLD2]-[rAPOBEC1 deaminase]-[UdgX]-[nCas9 domain];
[POLD2]-[rAPOBEC1 deaminase]-[UdgX]-[nCas9 domain]-[UdgX];
[POLD2]-[rAPOBEC1 deaminase]-[UdgX]-[nCas9 domain]-[RBMX];
[EXO1]-[rAPOBEC1 deaminase]-[UdgX]-[nCas9 domain];
[UdgX]-[Anc689 deaminase]-[UdgX]-[nCas9-NG domain]-[RBMX];
[UdgX]-[rAPOBEC1 deaminase]-[UdgX]-[nCas9-NG domain]; and
[UdgX]-[rAPOBEC1 deaminase]-[UdgX]-[HF-nCas9-NG domain],
   wherein each instance of “]-[” comprises an optional linker.

In particular embodiments, the fusion protein comprises the structure: [POLD2]-[rAPOBEC1 deaminase]-[UdgX]-[nCas9 domain]-[UdgX]; [UdgX]-[EE deaminase]-[UdgX]-[nCas9 domain]-[UdgX]; or [UdgX]-[Anc689 deaminase]-[UdgX]-[nCas9 domain]-[RBMX].

In some aspects, the present disclosure provides for methods of generating the transversion base editors and methods of using the disclosed transversion base editors or nucleic acid molecules encoding the transversion base editors in applications including editing a nucleic acid molecule, e.g., a genome. The specification provides methods for e editing a target nucleic acid molecule, e.g., a single nucleotide within a genome, with a base editing system described herein (e.g., in the form of a base editor as described herein, or a vector or construct encoding a base editor). Such methods involve transducing (e.g., via transfection) cells with a plurality of complexes each comprising a base editor (e.g., a fusion protein comprising a Cas9 nickase (nCas9) domain, a cytidine deaminase domain, and first and second UBP domains) and optionally a gRNA molecule. In some embodiments, the gRNA is bound to the napDNAbp domain (e.g., dCas9 domain) of the fusion protein. In certain embodiments, the methods involve the transfection of nucleic acid constructs (e.g., plasmids) that each (or together) encode the components of a complex of a base editor and/or gRNA.

In certain embodiments, the disclosed methods comprise contacting a double-stranded DNA sequence with a complex comprising a fusion protein disclosed herein and a guide RNA, wherein the double-stranded DNA comprises a target C:G nucleobase pair; thereby substituting the cytosine (C) of the C:G pair with a guanine. The disclosed methods may alternatively result in substitution of the guanine (G) of the C:G pair with a guanine derivative; such that the cell thereby subsequently substitutes the guanine derivative with a thymine during a subsequent round of replication.

In certain embodiments, the methods described herein further comprise cutting (or nicking) one strand of the double-stranded DNA, for example, the strand that includes the guanine (G) of the target C:G nucleobase pair opposite the strand containing the target cytosine (C) that is being mutated. This nicking step serves to direct mismatch repair machinery to the non-edited strand, ensuring that the modified nucleotide is not interpreted as a lesion by the cell's machinery. This nick may be created by the use of an nCas9.

The target nucleotide sequence may comprise a target sequence (e.g., a point mutation) associated with a disease, disorder, or condition, such as Ehlers-Danlos syndrome, Sotos syndrome, Cornelia de Lange syndrome, or a cancer. The target sequence may comprise a G to C point mutation associated with a disease, disorder, or condition, and wherein the excision and exchange of the mutant C base results in mismatch repair-mediated correction to a sequence that is not associated with a disease, disorder, or condition. Alternatively, the target sequence may comprise a C to G point mutation associated with a disease, disorder, or condition, and wherein the CGBE-mediated excision and exchange of the C base that is paired with the mutant G base results in mismatch repair-mediated correction to a sequence that is not associated with a disease, disorder, or condition.

The target sequence can encode a protein, and where the point mutation is in a codon and results in a change in the amino acid encoded by the mutant codon as compared to a wild-type codon. The target sequence may also be at a splice site, and the point mutation results in a change in the splicing of an mRNA transcript as compared to the wild-type transcript. In addition, the target may be at a non-coding sequence of a gene, such as a gene promoter or gene repressor, and the point mutation results in increased or decreased expression of the gene.

Exemplary target genes include the COL3A1 gene, the BRCA2 gene, the NSD1 gene, or the NIPBL gene. It will be appreciated that additional target genes for use in the disclosed methods include any human genes for which an oncogenic phenotype is frequently caused by G:C to C:G point mutations. COL3A1 is associated with Ehlers-Danlos syndrome; BRCA2 is associated with familial breast and ovarian cancer; NSD1 is associated with Sotos syndrome; and NIPBL is associated with Cornelia de Lange syndrome. Additional exemplary target sequences include the CTNBB1 gene, which is associated with cancer, and the DIS3L2 gene, which is associated with Perlmen syndrome. For some of these target genes, G:C to C:G point mutations introduce premature stop codons (UAA, UAG, UGA), resulting in nonsense mutations in protein coding regions. For all of the genetic disorders associated with the point mutations in these target genes, morbidity is high, and current treatment is not curative. Exemplary CGBEs disclosed herein correct these disease alleles in somatic cells, reducing or removing morbidity. In other embodiments, exemplary CGBEs disclosed herein may install disease-suppressing alleles in somatic cells.

Thus, in some aspects, the conversion of a mutant C results in correction of the nonsense mutation and restoration of the wild-type codon, which may result in the expression of a full-length, wild-type peptide sequence. For instance, the application of the base editors to target genetic sequences may induce a change in the mRNA transcript, such as restoring the mRNA transcript to a wild-type state.

The methods described herein may involve contacting a base editor with a target nucleotide sequence in vitro, ex vivo, or in vivo. In certain embodiments, this step of contacting occurs in a subject. In certain embodiments, the subject has been diagnosed with a disease, disorder, or condition, such as, but not limited to, a disease, disorder, or condition associated with a point mutation in the COL3A1 gene, the BRCA2 gene, the NSD1 gene, or the NIPBL gene.

In another aspect, the specification discloses a pharmaceutical composition comprising any one of the presently disclosed base editors (or fusion proteins). In one aspect, the specification discloses a pharmaceutical composition comprising any one of the presently disclosed complexes of fusion proteins and gRNA. In one aspect, the specification discloses a pharmaceutical composition comprising polynucleotides encoding the fusion proteins disclosed herein and polynucleotides encoding a gRNA, or polynucleotides encoding both. In another aspect, the specification discloses a pharmaceutical composition comprising any one of the presently disclosed vectors.

In some aspects, the disclosure provides base editors comprising one or more adenosine deaminase variants disclosed herein and a napDNAbp domain.

In some embodiments, the napDNAbp domain comprises a Cas homolog. The napDNAbp domain may be selected from a Cas9, a Cas9n, a dCas9, a CasX, a CasY, a C2c1, a C2c2, a C2c3, a GeoCas9, a CjCas9, a Cas12a, a Cas12b, a Cas12g, a Cas12h, a Cas12i, a Cas13a, a Cas13b, a Cas13c, a Cas13d, a Cas14, a Csn2, an xCas9, an SpCas9-NG, an SpCas9-NG-CP1041, an SpCas9-NG-VRQR, a high-fidelity Cas9 (HFCas9), a HF-nCas9, a HypaCas9, a HF-nCas9-NG, a Sniper-nCas9, an HF-Hypa-nCas9, an e-Cas9, an e-nCas9, an e-HF-Hypa-nCas9, an e-Hypa-Cas9, an e-Hypa-nCas9, an e-HF-nCas9, an LbCas12a, an AsCas12a, a Cas9-KKH, a circularly permuted Cas9, an Argonaute (Ago) domain, a SmacCas9, a Spy-macCas9, an SpCas9-VRQR, an SpCas9-NRRH, an SpaCas9-NRTH, an SpCas9-NRCH. In certain embodiments, the napDNAbp domain comprises or is a Cas9 domain or a Cas12a domain derived from S. pyogenes or S. aureus.

In some embodiments, the napDNAbp domain is derived from S. pyogenes and is selected from an nCas9, an nCas9-NG, an HF-Cas9, a HypaCas9, a HF-nCas9, a HF-nCas9-NG, an HF-Hypa-nCas9, an e-HF-Hypa-nCas9, and an e-HypaCas9. In particular embodiments, the napDNAbp domain is a HypaCas9, a HF-nCas9-NG, an HF-Hypa-nCas9, or an e-HF-HypanCas9.

It will be appreciated that all of of these disclosed Cas9 variants for use in the napDNAbp domains of the provided CGBEs can be engineered to have nickase activity (e.g., to contain a D10A substitution) or can be engineered to be nuclease-inactive (e.g., to contain D10A and H840A substitutions). It will be appreciated that these substitutions may be made in the wild-type Cas9 sequence of SEQ ID NO: 6, or at corresponding positions in any homologous Cas protein.

In some embodiments, the napDNAbp domain comprises a nuclease dead Cas9 (dCas9) domain, a Cas9 nickase (nCas9) domain, or a nuclease active Cas9 domain.

Further provided herein are methods of contacting any of the disclosed base editors with a nucleic acid molecule, e.g., a nucleic acid molecule (e.g., DNA) comprising a target sequence. In some embodiments of the disclosed methods, low off-target DNA and/or RNA editing effects are observed. In some embodiments, the nucleic acid molecule comprises a DNA, e.g., a single-stranded DNA or a double-stranded DNA. The target sequence of the nucleic acid molecule may comprise a target nucleobase pair containing a cytosine (C). The target sequence may be comprised within a genome, e.g., a human genome. The target sequence may comprise a sequence, e.g., a target sequence with point mutation, associated with a disease or disorder. The target sequence with a point mutation may be associated with Ehlers-Danlos syndrome, Sotos syndrome, Cornelia de Lange syndrome, or a cancer. In some embodiments, this editor may be used to target and revert single nucleotide polymorphisms (SNPs) in disease-relevant genes, which require C to G reversion.

In some aspects, the disclosure provides complexes comprising the CGBEs as described herein and one or more guide RNAs, e.g., a single-guide RNA (“sgRNA”), as well as compositions comprising any of these complexes. In addition, the present disclosure provides for nucleic acid molecules encoding and/or expressing the base editors as described herein, as well as expression vectors and constructs for expressing the base editors described herein and/or a gRNA (e.g., AAV vectors), host cells comprising any of said nucleic acid molecules and expression vectors and optionally vectors encoding one or more gRNAs, host cells comprising any of said base editors and optionally one or more gRNAs, and methods for delivering and/or administering nucleic acid-based embodiments described herein. In particular, the disclosure provides improved methods of delivery of the disclosed base editors, e.g., to a subject. Delivery of the disclosed base editors as RNPs, rather than DNA plasmids, typically increases on-target:off-target DNA editing ratios. Delivery of the disclosed CGBEs as mRNA molecules (e.g., using electroporation) may increases editing efficiencies.

Still further, the present disclosure provides for methods of creating the base editors described herein, as well as methods of using the base editors or nucleic acid molecules encoding any of these base editors in applications including editing a nucleic acid molecule, e.g., a genome. In certain embodiments, methods of engineering the base editors (or fusion proteins) provided herein involve a yeast system that may be utilized to evolve one or more components of a base editor (e.g., a polymerase domain). In certain embodiments, following the successful evolution of one or more components of the base editor (e.g., a polymerase domain), methods of making the base editors comprise recombinant protein expression methodologies and techniques known to those of skill in the art.

In some embodiments, the presently disclosed fusion proteins do not consist (or do not consist essentially of) a napDNAbp domain, a deaminase domain, and a single uracil binding protein. In some embodiments, the presently disclosed fusion proteins do not consist (or do not consist essentially of) a napDNAbp domain, a deaminase domain, a single uracil binding protein, and a nucleic acid polymerase (NAP) domain. In some embodiments, the presently disclosed fusion proteins do not consist (or do not consist essentially of) a napDNAbp domain, a deaminase domain, a single uracil binding protein, and a base exicision enzyme (BEE) domain. In some embodiments, the presently disclosed fusion proteins do not contain a base excision repair inhibitor. In some embodiments, the presently disclosed fusion proteins do not contain a mismatch repair protein.

Nucleic Acid Programmable DNA Binding Proteins (napDNAbp)

The base editors described herein comprise a nucleic acid programmable DNA binding (napDNAbp) domain. The napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp domain to localize and bind to a complementary sequence of the target strand. Binding of the napDNAbp domain to a complementary sequence enables the nucleobase modification domain (i.e., the cytidine deaminase domain) of the base editor to access and enzymatically deaminate a target cytosine base in the target strand.

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), the entire contents of which is hereby incorporated by reference.

Without wishing to be bound by any particular theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guideRNA protospacer then hybridizes to the “target strand.” This displaces a “non-target strand” that is complementary to the target strand, which forms the single strand region of the R-loop. In some embodiments, the napDNAbp includes one or more nuclease activities, which cuts the DNA leaving various types of lesions (e.g., a nick in one strand of the DNA). For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand.

The below description of various napDNAbps which can be used in connection with the disclosed cytidine deaminases and other fusion protein domains is not meant to be limiting in any way. The disclosed base editors may comprise the canonical SpCas9, or any ortholog Cas9 protein, or any variant Cas9 protein-including any naturally occurring variant, mutant, or otherwise engineered version of Cas9-that is known or which can be made or evolved through a directed evolutionary or otherwise mutagenic process. In various embodiments, the napDNAbp has a nickase activity, i.e., only cleave one strand of the target DNA sequence. In other embodiments, the napDNAbp has an inactive nuclease, e.g., are “dead” proteins. Other variant Cas9 proteins that may be used are those having a smaller molecular weight than the canonical SpCas9 (e.g., for easier delivery) or having modified or rearranged primary amino acid sequence (e.g., the circular permutant forms). The base editors described herein may also comprise Cas9 equivalents, including Cas12a/Cpf1 and Cas12b proteins. The napDNAbps used herein (e.g., SpCas9, SaCas9, or SaCas9 variant or SpCas9 variant) may also may also contain various modifications that alter/enhance their PAM specifies. The disclosure contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequence (set forth in SEQ ID NO: 326), a reference SaCas9 canonical sequence (set forth in SEQ ID NO: 377) or a reference Cas9 equivalent (e.g., Cas12a/Cpf1).

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

In some embodiments, the napDNAbp domain may comprise more than one napDNAbp protein. Accordingly, in some embodiments, any of the disclosed base editors may contain a first napDNAbp domain and a second napDNAbp domain. In some embodiments, the napDNAbp domain (or the first and second napDNAbp domain, respectively) comprises a first Cas homolog or variant and a second Cas homolog or variant (e.g., the first Cas comprises a Cas9, and the second Cas variant comprises a SpCas9-VRQR).

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference.

The term “Cas9” or “Cas9 domain” embraces any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular napDNAbp that is employed in the base editors of the disclosure.

Additional Cas9 sequences and structures are well known to those of skill in the art (see, e.g., “Complete genome sequence of an M1 strain of Streptococcus pyogenes.” Ferretti et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference), and also provided below.

Examples of Cas9 and Cas9 equivalents are provided; however, these specific examples are not meant to be limiting. The base editors of the present disclosure may use any suitable napDNAbp, including any suitable Cas9 or Cas9 equivalent.

Also useful in the present compositions and methods are nuclease-inactive Cpf1 (dCpf1) variants that may be used as a guide nucleotide sequence-programmable DNA-binding protein domain. The Cpf1 protein has a RuvC-like endonuclease domain that is similar to the RuvC domain of Cas9 but does not have a HNH endonuclease domain, and the N-terminal of Cpf1 does not have the alpha-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 (SEQ ID NO: 30) inactivates Cpf1 nuclease activity. In some embodiments, the dCpf1 of the present disclosure comprises mutations corresponding to D917A, E1006A, D1255A, D917A/E1006A, D917A/D1255A, E1006A/D1255A, or D917A/E1006A/D1255A in SEQ ID NO: 30, or corresponding mutation(s) in another Cpf1. It is to be understood that any mutations, e.g., substitution mutations, deletions, or insertions that inactivate the RuvC domain of Cpf1, may be used in accordance with the present disclosure.

In some embodiments, the nucleic acid programmable DNA binding protein (napDNAbp) of any of the fusion proteins provided herein may be a Cpf1 protein. In some embodiments, the Cpf1 protein is a Cpf1 nickase (nCpf1). In some embodiments, the Cpf1 protein is a nuclease inactive Cpf1 (dCpf1). In some embodiments, the Cpf1, the nCpf1, or the dCpf1 comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of SEQ ID NOs: 30-37. In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of SEQ ID NOs: 30-37, and comprises mutations corresponding to D917A, E1006A, D1255A, D917A/E1006A, D917A/D1255A, E1006A/D1255A, and or D917A/E1006A/D1255A in SEQ ID NO: 30 or corresponding mutation(s) in another Cpf1. In some embodiments, the dCpf1 comprises an amino acid sequence of any one SEQ ID NOs: 30-37. It should be appreciated that Cpf1 from other bacterial species may also be used in accordance with the present disclosure.

Wild type Francisella novicida Cpf1 (SEQ ID NO: 30) (D917, E1006, and D1255
are bolded and underlined)
(SEQ ID NO: 30)
MSIYQEFVNKYSLSKTLRFELIPQGKTLENIKARGLILDDEKRAKDYKKAKQIIDKYHQFFIEEI
LSSVCISEDLLQNYSDVYFKLKKSDDDNLQKDFKSAKDTIKKQISEYIKDSEKFKNLFNQNLID
AKKGQESDLILWLKQSKDNGIELFKANSDITDIDEALEIIKSFKGWTTYFKGFHENRKNVYSS
NDIPTSIIYRIVDDNLPKFLENKAKYESLKDKAPEAINYEQIKKDLAEELTFDIDYKTSEVNQR
VFSLDEVFEIANFNNYLNQSGITKFNTIIGGKFVNGENTKRKGINEYINLYSQQINDKTLKKYK
MSVLFKQILSDTESKSFVIDKLEDDSDVVTTMQSFYEQIAAFKTVEEKSIKETLSLLFDDLKAQ
KLDLSKIYFKNDKSLTDLSQQVFDDYSVIGTAVLEYITQQIAPKNLDNPSKKEQELIAKKTEK
AKYLSLETIKLALEEFNKHRDIDKQCRFEEILANFAAIPMIFDEIAQNKDNLAQISIKYQNQGK
KDLLQASAEDDVKAIKDLLDQTNNLLHKLKIFHISQSEDKANILDKDEHFYLVFEECYFELAN
IVPLYNKIRNYITQKPYSDEKFKLNFENSTLANGWDKNKEPDNTAILFIKDDKYYLGVMNKK
NNKIFDDKAIKENKGEGYKKIVYKLLPGANKMLPKVFFSAKSIKFYNPSEDILRIRNHSTHTK
NGSPQKGYEKFEFNIEDCRKFIDFYKQSISKHPEWKDFGFRFSDTQRYNSIDEFYREVENQGY
KLTFENISESYIDSVVNQGKLYLFQIYNKDFSAYSKGRPNLHTLYWKALFDERNLQDVVYKL
NGEAELFYRKQSIPKKITHPAKEAIANKNKDNPKKESVFEYDLIKDKRFTEDKFFFHCPITINF
KSSGANKFNDEINLLLKEKANDVHILSIDRGERHLAYYTLVDGKGNIIKQDTFNIIGNDRMKT
NYHDKLAAIEKDRDSARKDWKKINNIKEMKEGYLSQVVHEIAKLVIEYNAIVVFEDLNFGFK
RGRFKVEKQVYQKLEKMLIEKLNYLVFKDNEFDKTGGVLRAYQLTAPFETFKKMGKQTGII
YYVPAGFTSKICPVTGFVNQLYPKYESVSKSQEFFSKFDKICYNLDKGYFEFSFDYKNFGDKA
AKGKWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDADANGAYHI
GLKGLMLLGRIKNNQEGKKLNLVIKNEEYFEFVQNRNN
Francisella novicida Cpf1 D917A (SEQ ID NO: 31) (A917, E1006, and D1255 are
bolded and underlined)
(SEQ ID NO: 31)
MSIYQEFVNKYSLSKTLRFELIPQGKTLENIKARGLILDDEKRAKDYKKAKQIIDKYHQFFIEEI
LSSVCISEDLLQNYSDVYFKLKKSDDDNLQKDFKSAKDTIKKQISEYIKDSEKFKNLFNQNLID
AKKGQESDLILWLKQSKDNGIELFKANSDITDIDEALEIIKSFKGWTTYFKGFHENRKNVYSS
NDIPTSIIYRIVDDNLPKFLENKAKYESLKDKAPEAINYEQIKKDLAEELTFDIDYKTSEVNQR
VFSLDEVFEIANFNNYLNQSGITKFNTIIGGKFVNGENTKRKGINEYINLYSQQINDKTLKKYK
MSVLFKQILSDTESKSFVIDKLEDDSDVVTTMQSFYEQIAAFKTVEEKSIKETLSLLFDDLKAQ
KLDLSKIYFKNDKSLTDLSQQVFDDYSVIGTAVLEYITQQIAPKNLDNPSKKEQELIAKKTEK
AKYLSLETIKLALEEFNKHRDIDKQCRFEEILANFAAIPMIFDEIAQNKDNLAQISIKYQNQGK
KDLLQASAEDDVKAIKDLLDQTNNLLHKLKIFHISQSEDKANILDKDEHFYLVFEECYFELAN
IVPLYNKIRNYITQKPYSDEKFKLNFENSTLANGWDKNKEPDNTAILFIKDDKYYLGVMNKK
NNKIFDDKAIKENKGEGYKKIVYKLLPGANKMLPKVFFSAKSIKFYNPSEDILRIRNHSTHTK
NGSPQKGYEKFEFNIEDCRKFIDFYKQSISKHPEWKDFGFRFSDTQRYNSIDEFYREVENQGY
KLTFENISESYIDSVVNQGKLYLFQIYNKDFSAYSKGRPNLHTLYWKALFDERNLQDVVYKL
NGEAELFYRKQSIPKKITHPAKEAIANKNKDNPKKESVFEYDLIKDKRFTEDKFFFHCPITINF
KSSGANKFNDEINLLLKEKANDVHILSIARGERHLAYYTLVDGKGNIIKQDTFNIIGNDRMKT
NYHDKLAAIEKDRDSARKDWKKINNIKEMKEGYLSQVVHEIAKLVIEYNAIVVFEDLNFGFK
RGRFKVEKQVYQKLEKMLIEKLNYLVFKDNEFDKTGGVLRAYQLTAPFETFKKMGKQTGII
YYVPAGFTSKICPVTGFVNQLYPKYESVSKSQEFFSKFDKICYNLDKGYFEFSFDYKNFGDKA
AKGKWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDADANGAYHI
GLKGLMLLGRIKNNQEGKKLNLVIKNEEYFEFVQNRNN
Francisella novicida Cpf1 E1006A (SEQ ID NO: 32) (D917, A1006, and D1255
are bolded and underlined)
(SEQ ID NO: 32)
MSIYQEFVNKYSLSKTLRFELIPQGKTLENIKARGLILDDEKRAKDYKKAKQIIDKYHQFFIEEI
LSSVCISEDLLQNYSDVYFKLKKSDDDNLQKDFKSAKDTIKKQISEYIKDSEKFKNLFNQNLID
AKKGQESDLILWLKQSKDNGIELFKANSDITDIDEALEIIKSFKGWTTYFKGFHENRKNVYSS
NDIPTSIIYRIVDDNLPKFLENKAKYESLKDKAPEAINYEQIKKDLAEELTFDIDYKTSEVNQR
VFSLDEVFEIANFNNYLNQSGITKFNTIIGGKFVNGENTKRKGINEYINLYSQQINDKTLKKYK
MSVLFKQILSDTESKSFVIDKLEDDSDVVTTMQSFYEQIAAFKTVEEKSIKETLSLLFDDLKAQ
KLDLSKIYFKNDKSLTDLSQQVFDDYSVIGTAVLEYITQQIAPKNLDNPSKKEQELIAKKTEK
AKYLSLETIKLALEEFNKHRDIDKQCRFEEILANFAAIPMIFDEIAQNKDNLAQISIKYQNQGK
KDLLQASAEDDVKAIKDLLDQTNNLLHKLKIFHISQSEDKANILDKDEHFYLVFEECYFELAN
IVPLYNKIRNYITQKPYSDEKFKLNFENSTLANGWDKNKEPDNTAILFIKDDKYYLGVMNKK
NNKIFDDKAIKENKGEGYKKIVYKLLPGANKMLPKVFFSAKSIKFYNPSEDILRIRNHSTHTK
NGSPQKGYEKFEFNIEDCRKFIDFYKQSISKHPEWKDFGFRFSDTQRYNSIDEFYREVENQGY
KLTFENISESYIDSVVNQGKLYLFQIYNKDFSAYSKGRPNLHTLYWKALFDERNLQDVVYKL
NGEAELFYRKQSIPKKITHPAKEAIANKNKDNPKKESVFEYDLIKDKRFTEDKFFFHCPITINF
KSSGANKFNDEINLLLKEKANDVHILSIDRGERHLAYYTLVDGKGNIIKQDTFNIIGNDRMKT
NYHDKLAAIEKDRDSARKDWKKINNIKEMKEGYLSQVVHEIAKLVIEYNAIVVFADLNFGFK
RGRFKVEKQVYQKLEKMLIEKLNYLVFKDNEFDKTGGVLRAYQLTAPFETFKKMGKQTGII
YYVPAGFTSKICPVTGFVNQLYPKYESVSKSQEFFSKFDKICYNLDKGYFEFSFDYKNFGDKA
AKGKWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDADANGAYHI
GLKGLMLLGRIKNNQEGKKLNLVIKNEEYFEFVQNRNN
Francisella novicida Cpf1 D1255A (SEQ ID NO: 33) (D917, E1006, and A1255
are bolded and underlined)
(SEQ ID NO: 33)
MSIYQEFVNKYSLSKTLRFELIPQGKTLENIKARGLILDDEKRAKDYKKAKQIIDKYHQFFIEEI
LSSVCISEDLLQNYSDVYFKLKKSDDDNLQKDFKSAKDTIKKQISEYIKDSEKFKNLFNQNLID
AKKGQESDLILWLKQSKDNGIELFKANSDITDIDEALEIIKSFKGWTTYFKGFHENRKNVYSS
NDIPTSIIYRIVDDNLPKFLENKAKYESLKDKAPEAINYEQIKKDLAEELTFDIDYKTSEVNQR
VFSLDEVFEIANFNNYLNQSGITKFNTIIGGKFVNGENTKRKGINEYINLYSQQINDKTLKKYK
MSVLFKQILSDTESKSFVIDKLEDDSDVVTTMQSFYEQIAAFKTVEEKSIKETLSLLFDDLKAQ
KLDLSKIYFKNDKSLTDLSQQVEDDYSVIGTAVLEYITQQIAPKNLDNPSKKEQELIAKKTEK
AKYLSLETIKLALEEFNKHRDIDKQCRFEEILANFAAIPMIFDEIAQNKDNLAQISIKYQNQGK
KDLLQASAEDDVKAIKDLLDQTNNLLHKLKIFHISQSEDKANILDKDEHFYLVFEECYFELAN
IVPLYNKIRNYITQKPYSDEKFKLNFENSTLANGWDKNKEPDNTAILFIKDDKYYLGVMNKK
NNKIFDDKAIKENKGEGYKKIVYKLLPGANKMLPKVFFSAKSIKFYNPSEDILRIRNHSTHTK
NGSPQKGYEKFEFNIEDCRKFIDFYKQSISKHPEWKDFGFRFSDTQRYNSIDEFYREVENQGY
KLTFENISESYIDSVVNQGKLYLFQIYNKDFSAYSKGRPNLHTLYWKALFDERNLQDVVYKL
NGEAELFYRKQSIPKKITHPAKEAIANKNKDNPKKESVFEYDLIKDKRFTEDKFFFHCPITINF
KSSGANKFNDEINLLLKEKANDVHILSIDRGERHLAYYTLVDGKGNIIKQDTFNIIGNDRMKT
NYHDKLAAIEKDRDSARKDWKKINNIKEMKEGYLSQVVHEIAKLVIEYNAIVVFEDLNFGFK
RGRFKVEKQVYQKLEKMLIEKLNYLVFKDNEFDKTGGVLRAYQLTAPFETFKKMGKQTGII
YYVPAGFTSKICPVTGFVNQLYPKYESVSKSQEFFSKFDKICYNLDKGYFEFSFDYKNFGDKA
AKGKWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDAAANGAYHI
GLKGLMLLGRIKNNQEGKKLNLVIKNEEYFEFVQNRNN
Francisella novicida Cpf1 D917A/E1006A (SEQ ID NO: 34) (A917, A1006, and
D1255 are bolded and underlined)
(SEQ ID NO: 34)
MSIYQEFVNKYSLSKTLRFELIPQGKTLENIKARGLILDDEKRAKDYKKAKQIIDKYHQFFIEEI
LSSVCISEDLLQNYSDVYFKLKKSDDDNLQKDFKSAKDTIKKQISEYIKDSEKFKNLFNQNLID
AKKGQESDLILWLKQSKDNGIELFKANSDITDIDEALEIIKSFKGWTTYFKGFHENRKNVYSS
NDIPTSIIYRIVDDNLPKFLENKAKYESLKDKAPEAINYEQIKKDLAEELTFDIDYKTSEVNQR
VFSLDEVFEIANFNNYLNQSGITKFNTIIGGKFVNGENTKRKGINEYINLYSQQINDKTLKKYK
MSVLFKQILSDTESKSFVIDKLEDDSDVVTTMQSFYEQIAAFKTVEEKSIKETLSLLFDDLKAQ
KLDLSKIYFKNDKSLTDLSQQVEDDYSVIGTAVLEYITQQIAPKNLDNPSKKEQELIAKKTEK
AKYLSLETIKLALEEFNKHRDIDKQCRFEEILANFAAIPMIFDEIAQNKDNLAQISIKYQNQGK
KDLLQASAEDDVKAIKDLLDQTNNLLHKLKIFHISQSEDKANILDKDEHFYLVFEECYFELAN
IVPLYNKIRNYITQKPYSDEKFKLNFENSTLANGWDKNKEPDNTAILFIKDDKYYLGVMNKK
NNKIFDDKAIKENKGEGYKKIVYKLLPGANKMLPKVFFSAKSIKFYNPSEDILRIRNHSTHTK
NGSPQKGYEKFEFNIEDCRKFIDFYKQSISKHPEWKDFGFRFSDTQRYNSIDEFYREVENQGY
KLTFENISESYIDSVVNQGKLYLFQIYNKDFSAYSKGRPNLHTLYWKALFDERNLQDVVYKL
NGEAELFYRKQSIPKKITHPAKEAIANKNKDNPKKESVFEYDLIKDKRFTEDKFFFHCPITINF
KSSGANKFNDEINLLLKEKANDVHILSIARGERHLAYYTLVDGKGNIIKQDTFNIIGNDRMKT
NYHDKLAAIEKDRDSARKDWKKINNIKEMKEGYLSQVVHEIAKLVIEYNAIVVFADLNFGFK
RGRFKVEKQVYQKLEKMLIEKLNYLVFKDNEFDKTGGVLRAYQLTAPFETFKKMGKQTGII
YYVPAGFTSKICPVTGFVNQLYPKYESVSKSQEFFSKFDKICYNLDKGYFEFSFDYKNFGDKA
AKGKWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDADANGAYHI
GLKGLMLLGRIKNNQEGKKLNLVIKNEEYFEFVQNRNN
Francisella novicida Cpf1 D917A/D1255A (SEQ ID NO: 35) (A917, E1006, and
A1255 are bolded and underlined)
(SEQ ID NO: 35)
MSIYQEFVNKYSLSKTLRFELIPQGKTLENIKARGLILDDEKRAKDYKKAKQIIDKYHQFFIEEI
LSSVCISEDLLQNYSDVYFKLKKSDDDNLQKDFKSAKDTIKKQISEYIKDSEKFKNLFNQNLID
AKKGQESDLILWLKQSKDNGIELFKANSDITDIDEALEIIKSFKGWTTYFKGFHENRKNVYSS
NDIPTSIIYRIVDDNLPKFLENKAKYESLKDKAPEAINYEQIKKDLAEELTFDIDYKTSEVNQR
VFSLDEVFEIANFNNYLNQSGITKFNTIIGGKFVNGENTKRKGINEYINLYSQQINDKTLKKYK
MSVLFKQILSDTESKSFVIDKLEDDSDVVTTMQSFYEQIAAFKTVEEKSIKETLSLLFDDLKAQ
KLDLSKIYFKNDKSLTDLSQQVEDDYSVIGTAVLEYITQQIAPKNLDNPSKKEQELIAKKTEK
AKYLSLETIKLALEEFNKHRDIDKQCRFEEILANFAAIPMIFDEIAQNKDNLAQISIKYQNQGK
KDLLQASAEDDVKAIKDLLDQTNNLLHKLKIFHISQSEDKANILDKDEHFYLVFEECYFELAN
IVPLYNKIRNYITQKPYSDEKFKLNFENSTLANGWDKNKEPDNTAILFIKDDKYYLGVMNKK
NNKIFDDKAIKENKGEGYKKIVYKLLPGANKMLPKVFFSAKSIKFYNPSEDILRIRNHSTHTK
NGSPQKGYEKFEFNIEDCRKFIDFYKQSISKHPEWKDFGFRFSDTQRYNSIDEFYREVENQGY
KLTFENISESYIDSVVNQGKLYLFQIYNKDFSAYSKGRPNLHTLYWKALFDERNLQDVVYKL
NGEAELFYRKQSIPKKITHPAKEAIANKNKDNPKKESVFEYDLIKDKRFTEDKFFFHCPITINF
KSSGANKFNDEINLLLKEKANDVHILSIARGERHLAYYTLVDGKGNIIKQDTFNIIGNDRMKT
NYHDKLAAIEKDRDSARKDWKKINNIKEMKEGYLSQVVHEIAKLVIEYNAIVVFEDLNFGFK
RGRFKVEKQVYQKLEKMLIEKLNYLVFKDNEFDKTGGVLRAYQLTAPFETFKKMGKQTGII
YYVPAGFTSKICPVTGFVNQLYPKYESVSKSQEFFSKFDKICYNLDKGYFEFSFDYKNFGDKA
AKGKWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDAAANGAYHI
GLKGLMLLGRIKNNQEGKKLNLVIKNEEYFEFVQNRNN
Francisella novicida Cpf1 E1006A/D1255A (SEQ ID NO: 36) (D917, A1006, and
A1255 are bolded and underlined)
(SEQ ID NO: 36)
MSIYQEFVNKYSLSKTLRFELIPQGKTLENIKARGLILDDEKRAKDYKKAKQIIDKYHQFFIEEI
LSSVCISEDLLQNYSDVYFKLKKSDDDNLQKDFKSAKDTIKKQISEYIKDSEKFKNLFNQNLID
AKKGQESDLILWLKQSKDNGIELFKANSDITDIDEALEIIKSFKGWTTYFKGFHENRKNVYSS
NDIPTSIIYRIVDDNLPKFLENKAKYESLKDKAPEAINYEQIKKDLAEELTFDIDYKTSEVNQR
VFSLDEVFEIANFNNYLNQSGITKFNTIIGGKFVNGENTKRKGINEYINLYSQQINDKTLKKYK
MSVLFKQILSDTESKSFVIDKLEDDSDVVTTMQSFYEQIAAFKTVEEKSIKETLSLLFDDLKAQ
KLDLSKIYFKNDKSLTDLSQQVFDDYSVIGTAVLEYITQQIAPKNLDNPSKKEQELIAKKTEK
AKYLSLETIKLALEEFNKHRDIDKQCRFEEILANFAAIPMIFDEIAQNKDNLAQISIKYQNQGK
KDLLQASAEDDVKAIKDLLDQTNNLLHKLKIFHISQSEDKANILDKDEHFYLVFEECYFELAN
IVPLYNKIRNYITQKPYSDEKFKLNFENSTLANGWDKNKEPDNTAILFIKDDKYYLGVMNKK
NNKIFDDKAIKENKGEGYKKIVYKLLPGANKMLPKVFFSAKSIKFYNPSEDILRIRNHSTHTK
NGSPQKGYEKFEFNIEDCRKFIDFYKQSISKHPEWKDFGFRFSDTQRYNSIDEFYREVENQGY
KLTFENISESYIDSVVNQGKLYLFQIYNKDFSAYSKGRPNLHTLYWKALFDERNLQDVVYKL
NGEAELFYRKQSIPKKITHPAKEAIANKNKDNPKKESVFEYDLIKDKRFTEDKFFFHCPITINF
KSSGANKFNDEINLLLKEKANDVHILSIDRGERHLAYYTLVDGKGNIIKQDTFNIIGNDRMKT
NYHDKLAAIEKDRDSARKDWKKINNIKEMKEGYLSQVVHEIAKLVIEYNAIVVFADLNFGFK
RGRFKVEKQVYQKLEKMLIEKLNYLVFKDNEFDKTGGVLRAYQLTAPFETFKKMGKQTGII
YYVPAGFTSKICPVTGFVNQLYPKYESVSKSQEFFSKFDKICYNLDKGYFEFSFDYKNFGDKA
AKGKWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDAAANGAYHI
GLKGLMLLGRIKNNQEGKKLNLVIKNEEYFEFVQNRNN
Francisella novicida Cpf1 D917A/E1006A/D1255A (SEQ ID NO: 37) (A917,
A1006, and A1255 are bolded and underlined)
(SEQ ID NO: 37)
MSIYQEFVNKYSLSKTLRFELIPQGKTLENIKARGLILDDEKRAKDYKKAKQIIDKYHQFFIEEI
LSSVCISEDLLQNYSDVYFKLKKSDDDNLQKDFKSAKDTIKKQISEYIKDSEKFKNLFNQNLID
AKKGQESDLILWLKQSKDNGIELFKANSDITDIDEALEIIKSFKGWTTYFKGFHENRKNVYSS
NDIPTSIIYRIVDDNLPKFLENKAKYESLKDKAPEAINYEQIKKDLAEELTFDIDYKTSEVNQR
VFSLDEVFEIANFNNYLNQSGITKFNTIIGGKFVNGENTKRKGINEYINLYSQQINDKTLKKYK
MSVLFKQILSDTESKSFVIDKLEDDSDVVTTMQSFYEQIAAFKTVEEKSIKETLSLLFDDLKAQ
KLDLSKIYFKNDKSLTDLSQQVFDDYSVIGTAVLEYITQQIAPKNLDNPSKKEQELIAKKTEK
AKYLSLETIKLALEEFNKHRDIDKQCRFEEILANFAAIPMIFDEIAQNKDNLAQISIKYQNQGK
KDLLQASAEDDVKAIKDLLDQTNNLLHKLKIFHISQSEDKANILDKDEHFYLVFEECYFELAN
IVPLYNKIRNYITQKPYSDEKFKLNFENSTLANGWDKNKEPDNTAILFIKDDKYYLGVMNKK
NNKIFDDKAIKENKGEGYKKIVYKLLPGANKMLPKVFFSAKSIKFYNPSEDILRIRNHSTHTK
NGSPQKGYEKFEFNIEDCRKFIDFYKQSISKHPEWKDFGFRFSDTQRYNSIDEFYREVENQGY
KLTFENISESYIDSVVNQGKLYLFQIYNKDFSAYSKGRPNLHTLYWKALFDERNLQDVVYKL
NGEAELFYRKQSIPKKITHPAKEAIANKNKDNPKKESVFEYDLIKDKRFTEDKFFFHCPITINF
KSSGANKFNDEINLLLKEKANDVHILSIARGERHLAYYTLVDGKGNIIKQDTFNIIGNDRMKT
NYHDKLAAIEKDRDSARKDWKKINNIKEMKEGYLSQVVHEIAKLVIEYNAIVVFADLNFGFK
RGRFKVEKQVYQKLEKMLIEKLNYLVFKDNEFDKTGGVLRAYQLTAPFETFKKMGKQTGII
YYVPAGFTSKICPVTGFVNQLYPKYESVSKSQEFFSKFDKICYNLDKGYFEFSFDYKNFGDKA
AKGKWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDAAANGAYHI
GLKGLMLLGRIKNNQEGKKLNLVIKNEEYFEFVQNRNN

In some embodiments, the nucleic acid programmable DNA binding protein (napDNAbp) is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference. The sequence of Natronobacterium gregoryi Argonaute is provided in SEQ ID NO: 38.

Wild type Natronobacterium gregoryi Argonaute (SEQ ID NO: 38)
(SEQ ID NO: 38)
MTVIDLDSTTTADELTSGHTYDISVTLTGVYDNTDEQHPRMSLAFEQDNGERRYITLWKNTT
PKDVFTYDYATGSTYIFTNIDYEVKDGYENLTATYQTTVENATAQEVGTTDEDETFAGGEPL
DHHLDDALNETPDDAETESDSGHVMTSFASRDQLPEWTLHTYTLTATDGAKTDTEYARRTL
AYTVRQELYTDHDAAPVATDGLMLLTPEPLGETPLDLDCGVRVEADETRTLDYTTAKDRLL
ARELVEEGLKRSLWDDYLVRGIDEVLSKEPVLTCDEFDLHERYDLSVEVGHSGRAYLHINFR
HRFVPKLTLADIDDDNIYPGLRVKTTYRPRRGHIVWGLRDECATDSLNTLGNQSVVAYHRN
NQTPINTDLLDAIEAADRRVVETRRQGHGDDAVSFPQELLAVEPNTHQIKQFASDGFHQQAR
SKTRLSASRCSEKAQAFAERLDPVRLNGSTVEFSSEFFTGNNEQQLRLLYENGESVLTFRDGA
RGAHPDETFSKGIVNPPESFEVAVVLPEQQADTCKAQWDTMADLLNQAGAPPTRSETVQYD
AFSSPESISLNVAGAIDPSEVDAAFVVLPPDQEGFADLASPTETYDELKKALANMGIYSQMAY
FDRFRDAKIFYTRNVALGLLAAAGGVAFTTEHAMPGDADMFIGIDVSRSYPEDGASGQINIA
ATATAVYKDGTILGHSSTRPQLGEKLQSTDVRDIMKNAILGYQQVTGESPTHIVIHRDGFMNE
DLDPATEFLNEQGVEYDIVEIRKQPQTRLLAVSDVQYDTPVKSIAAINQNEPRATVATFGAPE
YLATRDGGGLPRPIQIERVAGETDIETLTRQVYLLSQSHIQVHNSTARLPITTAYADQASTHAT
KGYLVQTGAFESNVGFL

In some embodiments, the napDNAbp is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piezophila Argunaute (MpAgo) protein. The CRISPR-associated Marinitoga piezophila Argunaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other argonaute proteins may be used, and are within the scope of this disclosure.

In some embodiments, the nucleic acid programmable DNA binding protein (napDNAbp) is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins provided herein may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring C2c1, C2c2, or C2c3 protein. In some embodiments, the napDNAbp is a naturally-occurring C2c1, C2c2, or C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of SEQ ID NOs: 39-40. It should be appreciated that C2c1, C2c2, or C2c3 from other bacterial species may also be used in accordance with the present disclosure.

C2c1 (uniprot.org/uniprot/T0D7A2#)
sp|T0D7A2|C2C1_ALIAG CRISPR-associated endonuclease C2c1
OS = Alicyclobacillus acidoterrestris (strain ATCC 49025/DSM 3922/CIP 106132/
NCIMB 13137/GD3B) GN = c2c1 PE = 1 SV = 1
(SEQ ID NO: 39)
MAVKSIKVKLRLDDMPEIRAGLWKLHKEVNAGVRYYTEWLSLLRQENLYRRSPNGDGEQEC
DKTAEECKAELLERLRARQVENGHRGPAGSDDELLQLARQLYELLVPQAIGAKGDAQQIARKFLSPLA
DKDAVGGLGIAKAGNKPRWVRMREAGEPGWEEEKEKAETRKSADRTADVLRALADFGLKPLMRVY
TDSEMSSVEWKPLRKGQAVRTWDRDMFQQAIERMMSWESWNQRVGQEYAKLVEQKNRFEQKNFVG
QEHLVHLVNQLQQDMKEASPGLESKEQTAHYVTGRALRGSDKVFEKWGKLAPDAPFDLYDAEIKNV
QRRNTRRFGSHDLFAKLAEPEYQALWREDASFLTRYAVYNSILRKLNHAKMFATFTLPDATAHPIWTR
FDKLGGNLHQYTFLFNEFGERRHAIRFHKLLKVENGVAREVDDVTVPISMSEQLDNLLPRDPNEPIALY
FRDYGAEQHFTGEFGGAKIQCRRDQLAHMHRRRGARDVYLNVSVRVQSQSEARGERRPPYAAVFRLV
GDNHRAFVHFDKLSDYLAEHPDDGKLGSEGLLSGLRVMSVDLGLRTSASISVFRVARKDELKPNSKGR
VPFFFPIKGNDNLVAVHERSQLLKLPGETESKDLRAIREERQRTLRQLRTQLAYLRLLVRCGSEDVGRR
ERSWAKLIEQPVDAANHMTPDWREAFENELQKLKSLHGICSDKEWMDAVYESVRRVWRHMGKQVR
DWRKDVRSGERPKIRGYAKDVVGGNSIEQIEYLERQYKFLKSWSFFGKVSGQVIRAEKGSRFAITLREH
IDHAKEDRLKKLADRIIMEALGYVYALDERGKGKWVAKYPPCQLILLEELSEYQFNNDRPPSENNQLM
QWSHRGVFQELINQAQVHDLLVGTMYAAFSSRFDARTGAPGIRCRRVPARCTQEHNPEPFPWWLNKF
VVEHTLDACPLRADDLIPTGEGEIFVSPFSAEEGDFHQIHADLNAAQNLQQRLWSDFDISQIRLRCDWG
EVDGELVLIPRLTGKRTADSYSNKVFYTNTGVTYYERERGKKRRKVFAQEKLSEEEAELLVEADEARE
KSVVLMRDPSGIINRGNWTRQKEFWSMVNQRIEGYLVKQIRSRVPLQDSACENTGDI
C2c2 (uniprot.org/uniprot/P0DOC6)
>sp|P0DOC6|C2C2_LEPSD CRISPR-associated endoribonuclease C2c2
OS = Leptotrichia shahii (strain DSM 19757/CCUG 47503/CIP 107916/JCM 16776/
LB37) GN = c2c2 PE = 1 SV = 1
(SEQ ID NO: 40)
MGNLFGHKRWYEVRDKKDFKIKRKVKVKRNYDGNKYILNINENNNKEKIDNNKFIRKYINYK
KNDNILKEFTRKFHAGNILFKLKGKEGIIRIENNDDFLETEEVVLYIEAYGKSEKLKALGITKKKIIDEAIR
QGITKDDKKIEIKRQENEEEIEIDIRDEYTNKTLNDCSIILRIIENDELETKKSIYEIFKNINMSLYKIIEKIIE
NETEKVFENRYYEEHLREKLLKDDKIDVILTNFMEIREKIKSNLEILGFVKFYLNVGGDKKKSKNKKML
VEKILNINVDLTVEDIADFVIKELEFWNITKRIEKVKKVNNEFLEKRRNRTYIKSYVLLDKHEKFKIERE
NKKDKIVKFFVENIKNNSIKEKIEKILAEFKIDELIKKLEKELKKGNCDTEIFGIFKKHYKVNFDSKKFSK
KSDEEKELYKIIYRYLKGRIEKILVNEQKVRLKKMEKIEIEKILNESILSEKILKRVKQYTLEHIMYLGKL
RHNDIDMTTVNTDDFSRLHAKEELDLELITFFASTNMELNKIFSRENINNDENIDFFGGDREKNYVLDK
KILNSKIKIIRDLDFIDNKNNITNNFIRKFTKIGTNERNRILHAISKERDLQGTQDDYNKVINIIQNLKISDE
EVSKALNLDVVFKDKKNIITKINDIKISEENNNDIKYLPSFSKVLPEILNLYRNNPKNEPFDTIETEKIVLN
ALIYVNKELYKKLILEDDLEENESKNIFLQELKKTLGNIDEIDENIIENYYKNAQISASKGNNKAIKKYQK
KVIECYIGYLRKNYEELFDFSDFKMNIQEIKKQIKDINDNKTYERITVKTSDKTIVINDDFEYIISIFALLNS
NAVINKIRNRFFATSVWLNTSEYQNIIDILDEIMQLNTLRNECITENWNLNLEEFIQKMKEIEKDFDDFKI
QTKKEIFNNYYEDIKNNILTEFKDDINGCDVLEKKLEKIVIFDDETKFEIDKKSNILQDEQRKLSNINKKD
LKKKVDQYIKDKDQEIKSKILCRIIFNSDFLKKYKKEIDNLIEDMESENENKFQEIYYPKERKNELYIYKK
NLFLNIGNPNFDKIYGLISNDIKMADAKFLFNIDGKNIRKNKISEIDAILKNLNDKLNGYSKEYKEKYIKK
LKENDDFFAKNIQNKNYKSFEKDYNRVSEYKKIRDLVEFNYLNKIESYLIDINWKLAIQMARFERDMH
YIVNGLRELGIIKLSGYNTGISRAYPKRNGSDGFYTTTAYYKFFDEESYKKFEKICYGFGIDLSENSEINK
PENESIRNYISHFYIVRNPFADYSIAEQIDRVSNLLSYSTRYNNSTYASVFEVFKKDVNLDYDELKKKFK
LIGNNDILERLMKPKKVSVLELESYNSDYIKNLIIELLTKIENTNDTL

### Cas9 Domains of the Disclosed Base Editors

In some aspects, a nucleic acid programmable DNA binding protein (napDNAbp) is a Cas9 domain. Non-limiting, exemplary Cas9 domains are provided herein. The Cas9 domain may be a nuclease active Cas9 domain, a nuclease inactive Cas9 domain, or a Cas9 nickase. In some embodiments, the Cas9 domain is a nuclease active domain. For example, the Cas9 domain may be a Cas9 domain that cuts both strands of a duplexed nucleic acid (e.g., both strands of a duplexed DNA molecule). In some embodiments, the Cas9 domain comprises any one of the amino acid sequences as set forth in SEQ ID NOs: 4-29, 724-736. In some embodiments the Cas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any Cas9 provided herein, or to one of the amino acid sequences set forth in SEQ ID NOs: 4-29, 724-736. In some embodiments, the Cas9 domain comprises an amino acid sequence that has 1,2, 3,4, 5, 6,7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any Cas9 provided herein, or to any one of the amino acid sequences set forth in SEQ ID NOs: 4-29, 724-736. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous (or consecutive) amino acid residues as compared to any Cas9 provided herein or any one of the amino acid sequences set forth in SEQ ID NOs: 4-29, 724-736.

In some aspects, the CGBEs of the disclosure include a napDNAbp domain that is a Cas9 variant having a higher targeting specificity than the Cas9 domains of previously disclosed CGBEs. In some embodiments, the napDNAbp domain is selected from a HypaCas9, a HF-nCas9-NG, a Sniper-nCas9, an HF-Hypa-nCas9, an e-Cas9, an e-HF-Hypa-nCas9, and an e-Hypa-Cas9. In some aspects, the napDNAbp domain is selected from an HF-nCas9-NG, an HF-Hypa-nCas9, and an e-HF-Hypa-nCas9. In some embodiments, the CGBEs of the disclosure may comprise: (i) a napDNAbp domain, (ii) a cytidine deaminase domain, (iii) a first uracil binding protein (UBP) domain, and (iv) a DNA repair protein; or (i) a napDNAbp domain, (ii) a cytidine deaminase domain, (iii) a first UBP domain, and (iv) a second UBP domain, wherein the napDNAbp domain is selected from a HypaCas9, a HF-nCas9-NG, a Sniper-nCas9, an HF-Hypa-nCas9, an e-Cas9, an e-HF-Hypa-nCas9, and an e-Hypa-Cas9. In some embodiments, the napDNAbp domain of any of the disclosed CGBEs comprises an amino acid sequence that is at least 85%, 90%, 92.5%, 95%, 97%, 98%, or 99% identical to any of the sequences set forth as SEQ ID NOs: 724-736. In some embodiments, the napDNAbp domain of any of the disclosed CGBEs is selected from SEQ ID NOs: 724-736.

In some embodiments, the napDNAbp of any of the disclosed base editors comprises the amino acid sequence of SEQ ID NO: 9 (dCas9). In some embodiments, the napDNAbp of any of the disclosed base editors comprises the amino acid sequence of SEQ ID NO: 16 (nCas9).

In some embodiments, the disclosed base editors may comprise a catalytically inactive, or “dead,” napDNAbp domain. Exemplary catalytically inactive domains in the disclosed base editors are dead S. pyogenes Cas9 (dSpCas9), dead S. aureus Cas9 (dSaCas9) and dead Lachnospiraceae bacterium Cas12a (dLbCas12a).

In certain embodiments, the base editors described herein may include a dead Cas9, e.g., dead SpCas9, which has no nuclease activity due to one or more mutations that inactivate both nuclease domains of SpCas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

In certain embodiments, the base editors described herein may include a dead Cas9, e.g., dead SpCas9, which has no nuclease activity due to one or more mutations that inactivate both nuclease domains of SaCas9, namely the RuvC domain (which cleaves the non-protospacer DNA strand) and HNH domain (which cleaves the protospacer DNA strand). The D10A and N580A mutations in the wild-type S. aureus Cas9 amino acid sequence may be used to form a dSaCas9. Accordingly, in some embodiments, the napDNAbp domain of the base editors provided herein comprises a dSaCas9 that has D10A and N580A mutations relative to the wild-type SaCas9 sequence (SEQ ID NO: 377).

In some embodiments, the Cas9 domain is a nuclease-inactive Cas9 domain (dCas9). For example, the dCas9 domain may bind to a duplexed nucleic acid molecule (e.g., via a gRNA molecule) without cleaving either strand of the duplexed nucleic acid molecule. In some embodiments, the nuclease-inactive dCas9 domain comprises a D10X mutation and a H840X mutation of the amino acid sequence set forth in SEQ ID NO: 6, or a corresponding mutation in any Cas9 provided herein, such as one of the amino acid sequences provided in SEQ ID NOs: 4-26, wherein X is any amino acid change. In some embodiments, the nuclease-inactive dCas9 domain comprises a D10A mutation and a H840A mutation of the amino acid sequence set forth in SEQ ID NO: 6, or a corresponding mutation in any Cas9 provided herein, such as any one of the amino acid sequences provided in SEQ ID NOs: 4-26. As one example, a nuclease-inactive Cas9 domain comprises the amino acid sequence set forth in SEQ ID NO: 9 (Cloning vector pPlatTET-gRNA2, Accession No. BAV54124).

(SEQ ID NO: 9)
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFL
KSDGFANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVENTQLQ
NEKLYLYYLQNGRDMYVDQELDINRLSDYDVDAIVPQSFLKDDSIDNKVLTRSDKNRGKSD
NVPSEEVVKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHRDKPIRE
QAENIIHLFTLTNLGAPAAFKYFDTTIDRKRYTSTKEVLDATLIHQSITGLYETRIDLSQLGGD;

see, e.g., Qi et al., “Repurposing CRISPR as an RNA-guided platform for sequence-specific control of gene expression.” Cell. 2013; 152(5):1173-83, the entire contents of which are incorporated herein by reference).

In some embodiments, the napDNAbp domain of any of the disclosed base editors comprises a dead S. pyogenes Cas9 (dSpCas9). In some embodiments, the napDNAbp domain of any of the disclosed based editors is comprises at least 80%, at least 85%, at least 90%, at least 95%, or at least 99% sequence identity to SEQ ID NO: 8 or 9. In some embodiments, the napDNAbp domain of any of the disclosed base editors comprises the amino acid sequence of SEQ ID NO: 8 or 9.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference). In some embodiments the dCas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the dCas9 domains provided herein. In some embodiments, the Cas9 domain comprises an amino acid sequences that has 1,2, 3,4, 5, 6,7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 7, 8, 9, or 22. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NOs: 7, 8, 9, or 22.

In some embodiments, the disclosed CGBEs may comprise a napDNAbp domain that comprises a nickase. In some embodiments, the CGBEs described herein comprise a Cas9 nickase. The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof.

In some embodiments, the Cas9 domain is a Cas9 nickase. The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840 of SEQ ID NO: 6, or a mutation in any Cas9 provided herein, such as any one of SEQ ID NOs: 4-26. For example, a Cas9 nickase may comprise the amino acid sequence as set forth in SEQ ID NO: 10, 13, 16, or 21. In some embodiments, the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10 of SEQ ID NO: 6, or a corresponding mutation in any Cas9 provided herein, such as any one of SEQ ID NOs: 4-26. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

In some embodiments, the napDNAbp domain of any of the disclosed base editors comprises an S. pyogenes Cas9 nickase (SpCas9n). In some embodiments, the napDNAbp domain of any of the disclosed based editors is comprises at least 80%, at least 85%, at least 90%, at least 95%, or at least 99% sequence identity to SEQ ID NO: 10 or 16. In some embodiments, the napDNAbp domain of any of the disclosed base editors comprises the amino acid sequence of SEQ ID NO: 10 or 16.

In some embodiments, the napDNAbp domain of any of the disclosed base editors comprises an S. aureus Cas9 nickase (SaCas9n). In some embodiments, the napDNAbp domain of any of the disclosed based editors is comprises at least 80%, at least 85%, at least 90%, at least 95%, or at least 99% sequence identity to SEQ ID NO: 13. In some embodiments, the napDNAbp domain of any of the disclosed base editors comprises the amino acid sequence of SEQ ID NO: 13.

Cas9 Domains with Reduced PAM Exclusivity

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. This may limit the ability to edit desired bases within a genome. In some embodiments, the base editing fusion proteins provided herein need to be positioned at a precise location, for example, where a target base is within a 4 base region (e.g., a “deamination window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. In some embodiments, the deamination window is within a 2, 3, 4, 5, 6, 7, 8, 9, or 10 base region. In some embodiments, the deamination window is 5, 6, 7, 8, 9, 10, 11, 12, 13, 14, 15, 16, 17, 18, 19, 20, 21, 22, 23, 24, or 25 bases upstream of the PAM. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises the amino acid sequence SEQ ID NO: 12. In some embodiments, the SaCas9 comprises a N579X mutation of SEQ ID NO: 12, or a corresponding mutation in any of the amino acid sequences provided in SEQ ID NOs: 13-14, wherein X is any amino acid except for N. In some embodiments, the SaCas9 comprises a N579A mutation of SEQ ID NO: 12, or a corresponding mutation in any of the amino acid sequences provided in SEQ ID NOs: 13-14.

In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT (SEQ ID NO: 223) PAM sequence, where N=A, T, C, or G, and R=A or G. In some embodiments, the SaCas9 domain comprises one or more of E781X, N967X, and R1014X mutation of SEQ ID NO: 12, or a corresponding mutation in any of the amino acid sequences provided in SEQ ID NOs: 13-14, wherein X is any amino acid. In some embodiments, the SaCas9 domain comprises one or more of a E781K, a N967K, and a R1014H mutation of SEQ ID NO: 12, or one or more corresponding mutation in any of the amino acid sequences provided in SEQ ID NOs: 13-14. In some embodiments, the SaCas9 domain comprises a E781K, a N967K, or a R1014H mutation of SEQ ID NO: 12, or corresponding mutations in any of the amino acid sequences provided in SEQ ID NOs: 13-14.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 12-14. In some embodiments, the Cas9 domain of any of the fusion proteins provided herein comprises the amino acid sequence of any one of SEQ ID NOs: 12-14. In some embodiments, the Cas9 domain of any of the fusion proteins provided herein consists of the amino acid sequence of any one of SEQ ID NOs: 12-14.

### Exemplary SaCas9 Sequence

(SEQ ID NO: 12)
KRNYILGLDIGITSVGYGIIDYETRDVIDAGVRLFKEANVENNEGRRSKRGARRLKRR
RRHRIQRVKKLLFDYNLLTDHSELSGINPYEARVKGLSQKLSEEEFSAALLHLAKRRG
VHNVNEVEEDTGNELSTKEQISRNSKALEEKYVAELQLERLKKDGEVRGSINRFKTS
DYVKEAKQLLKVQKAYHQLDQSFIDTYIDLLETRRTYYEGPGEGSPFGWKDIKEWY
EMLMGHCTYFPEELRSVKYAYNADLYNALNDLNNLVITRDENEKLEYYEKFQIIENV
FKQKKKPTLKQIAKEILVNEEDIKGYRVTSTGKPEFTNLKVYHDIKDITARKEIIENAE
LLDQIAKILTIYQSSEDIQEELTNLNSELTQEEIEQISNLKGYTGTHNLSLKAINLILDEL
WHTNDNQIAIFNRLKLVPKKVDLSQQKEIPTTLVDDFILSPVVKRSFIQSIKVINAIIKK
YGLPNDIIIELAREKNSKDAQKMINEMQKRNRQTNERIEEIIRTTGKENAKYLIEKIKL
HDMQEGKCLYSLEAIPLEDLLNNPFNYEVDHIIPRSVSFDNSFNNKVLVKQEENSKK
GNRTPFQYLSSSDSKISYETFKKHILNLAKGKGRISKTKKEYLLEERDINRFSVQKDFI
NRNLVDTRYATRGLMNLLRSYFRVNNLDVKVKSINGGFTSFLRRKWKFKKERNKG
YKHHAEDALIIANADFIFKEWKKLDKAKKVMENQMFEEKQAESMPEIETEQEYKEIF
ITPHQIKHIKDFKDYKYSHRVDKKPNRELINDTLYSTRKDDKGNTLIVNNLNGLYDK
DNDKLKKLINKSPEKLLMYHHDPQTYQKLKLIMEQYGDEKNPLYKYYEETGNYLTK
YSKKDNGPVIKKIKYYGNKLNAHLDITDDYPNSRNKVVKLSLKPYRFDVYLDNGVY
KFVTVKNLDVIKKENYYEVNSKCYEEAKKLKKISNQAEFIASFYNNDLIKINGELYRV
IGVNNDLLNRIEVNMIDITYREYLENMNDKRPPRIIKTIASKTQSIKKYSTDILGNLYE
VKSKKHPQIIKKG

Residue N579 of SEQ ID NO: 12, which is underlined and in bold, may be mutated (e.g., to a A579) to yield a SaCas9 nickase.

### Exemplary SaCas9n Sequence

(SEQ ID NO: 13)
KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEEASKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMN
DKRPPRIIKTIASKTQSIKKYSTDILGNLYEVKSKKHPQIIKKG.

Residue A579 of SEQ ID NO: 13, which can be mutated from N579 of SEQ ID NO: 12 to yield a SaCas9 nickase, is underlined and in bold.

(SEQ ID NO: 14)
KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEEASKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYKNDLIKINGELYRVIGVNNDLLNRIEVNMIDITYREYLENMN
DKRPPHIIKTIASKTQSIKKYSTDILGNLYEVKSKKHPQIIKKG.

Residue A579 of SEQ ID NO: 14, which can be mutated from N579 of SEQ ID NO: 12 to yield a SaCas9 nickase, is underlined and in bold. Residues K781, K967, and H1014 of SEQ ID NO: 14, which can be mutated from E781, N967, and R1014 of SEQ ID NO: 12 to yield a SaKKH Cas9 are underlined and in italics.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises the amino acid sequence SEQ ID NO: 15. In some embodiments, the SpCas9 comprises a D9X mutation of SEQ ID NO: 15, or a corresponding mutation in any Cas9, such as any of the amino acid sequences provided in SEQ ID NOs: 4-26, wherein X is any amino acid except for D. In some embodiments, the SpCas9 comprises a D9A mutation of SEQ ID NO: 15, or a corresponding mutation in any Cas9 provided herein, such as any of the amino acid sequences provided in SEQ ID NOs: 4-26. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having a NGG, a NGA, or a NGCG PAM sequence. In some embodiments, the SpCas9 domain comprises one or more of a D1134X, a R1334X, and a T1336X mutation of SEQ ID NO: 15, or a corresponding mutation in any Cas9 provided herein, such as any of the amino acid sequences provided in SEQ ID NOs: 4-26, wherein X is any amino acid. In some embodiments, the SpCas9 domain comprises one or more of a D1134E, R1334Q, and T1336R mutation of SEQ ID NO: 15, or a corresponding mutation in any Cas9 provided herein, such as any of the amino acid sequences provided in SEQ ID NOs: 4-26. In some embodiments, the SpCas9 domain comprises a D1 134E, a R1334Q, and a T1336R mutation of SEQ ID NO: 15, or corresponding mutations in any Cas9 provided herein, such as any of the amino acid sequences provided in SEQ ID NOs: 4-26. In some embodiments, the SpCas9 domain comprises one or more of a D1134X, a R1334X, and a T1336X mutation of SEQ ID NO: 15, or a corresponding mutation in any Cas9 provided herein, such as any of the amino acid sequences provided in SEQ ID NOs: 4-26, wherein X is any amino acid. In some embodiments, the SpCas9 domain comprises one or more of a D1134V, a R1334Q, and a T1336R mutation of SEQ ID NO: 15, or a corresponding mutation in any Cas9 provided herein, such as any of the amino acid sequences provided in SEQ ID NOs: 4-26. In some embodiments, the SpCas9 domain comprises a D1 134V, a R1334Q, and a T1336R mutation of SEQ ID NO: 15, or corresponding mutations in any Cas9 provided herein, such as any of the amino acid sequences provided in SEQ ID NOs: 4-26. In some embodiments, the SpCas9 domain comprises one or more of a D1134X, a G1217X, a R1334X, and a T1336X mutation of SEQ ID NO: 15, or a corresponding mutation in any Cas9 provided herein, such as any one of the amino acid sequences provided in SEQ ID NOs: 4-26, wherein X is any amino acid. In some embodiments, the SpCas9 domain comprises one or more of a D1134V, a G1217R, a R1334Q, and a T1336R mutation of SEQ ID NO: 15, or a corresponding mutation in any Cas9 provided herein, such as any of the amino acid sequences provided in SEQ ID NOs: 4-26. In some embodiments, the SpCas9 domain comprises a D1134V, a G1217R, a R1334Q, and a T1336R mutation of SEQ ID NO: 15, or corresponding mutations in any Cas9 provided herein, such as any one of the amino acid sequences provided in SEQ ID NOs: 4-26.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 15-19. In some embodiments, the Cas9 domain of any of the fusion proteins provided herein comprises the amino acid sequence of any one of SEQ ID NOs: 15-19. In some embodiments, the Cas9 domain of any of the fusion proteins provided herein consists of the amino acid sequence of any one of SEQ ID NOs: 15-19.

### Exemplary SpCas9

(SEQ ID NO: 15)
DKKYSIGLDIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPL
SASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNT
KYDENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD

### Exemplary SpCas9n

(SEQ ID NO: 16)
DKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPL
SASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNT
KYDENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD

### Exemplary SpEQR Cas9

(SEQ ID NO: 17)
DKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPL
SASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNT
KYDENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKKDWDPK
KYGGFESPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFTLTN
LGAPAAFKYFDTTIDRKQYRSTKEVLDATLIHQSITGLYETRIDLSQLGGD

Residues E1134, Q1334, and R1336 of SEQ ID NO: 17, which can be mutated from D1134, R1334, and T1336 of SEQ ID NO: 15 to yield a SpEQR Cas9, are underlined and in bold.

### Exemplary SpVQR Cas9

(SEQ ID NO: 18)
DKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPL
SASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNT
KYDENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKKDWDPK
KYGGFVSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFTLTN
LGAPAAFKYFDTTIDRKQYRSTKEVLDATLIHQSITGLYETRIDLSQLGGD

Residues V1134, Q1334, and R1336 of SEQ ID NO: 18, which can be mutated from D1134, R1334, and T1336 of SEQ ID NO: 15 to yield a SpVQR Cas9, are underlined and in bold.

### Exemplary SpVRER Cas9

(SEQ ID NO: 19)
DKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPL
SASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNT
KYDENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKKDWDPK
KYGGFVSPTVAYSVLVVAKVEKGKSKKLKSVKELLGITIMERSSFEKNPIDFLEAKGYKEVK
KDLIIKLPKYSLFELENGRKRMLASARELQKGNELALPSKYVNFLYLASHYEKLKGSPEDNE
QKQLFVEQHKHYLDEIIEQISEFSKRVILADANLDKVLSAYNKHRDKPIREQAENIIHLFTLTN
LGAPAAFKYFDTTIDRKEYRSTKEVLDATLIHQSITGLYETRIDLSQLGGD

Residues V1134, R1217, Q1334, and R1336 of SEQ ID NO: 19, which can be mutated from D1134, G1217, R1334, and T1336 of SEQ ID NO: 15 to yield a SpVRER Cas9, are underlined and in bold.

In some embodiments, the disclosure provides napDNAbp domains that comprise SpCas9 variants that recognize and work best with NRRH, NRCH, and NRTH PAMs. See International Application No. PCT/US2019/47996, which published as International Publication No. WO 2020/041751 on Feb. 27, 2020, incorporated by reference herein. In some embodiments, the disclosed base editors comprise a napDNAbp domain selected from SpCas9-NRRH, SpCas9-NRTH, and SpCas9-NRCH.

In some embodiments, the disclosed base editors comprise a napDNAbp domain that has a sequence that is at least 90%, at least 95%, at least 98%, or at least 99% identical to SpCas9-NRRH. In some embodiments, the disclosed base editors comprise a napDNAbp domain that comprises SpCas9-NRRH. The SpCas9-NRRH has an amino acid sequence as presented in SEQ ID NO: 435 (underligned residues are mutated relative to SpCas9, as set

(SEQ ID NO: 435)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMVKRYDEHHQDLTLLKALVRQQLPE
KYKEIFFDQSKNGYAGYIDGGASQEEFYKFIKPILEKMDGTEELLVKLNREDLLRKQR
TFDNGIIPHQIHLGELHAILRRQGDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRLRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGGHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKGNSDKLIARKKDWDPKKYGGFNSPTAAYSVLVV
AKVEKGKSKKLKSVKELLGITIMERSSFEKNPIGFLEAKGYKEVKKDLIIKLPKYSLFE
LENGRKRMLASAGVLHKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGV
PAAFKYFDTTIDKKRYTSTKEVLDATLIHQSITGLYETRIDLSQLGGD

In some embodiments, the disclosed base editors comprise a napDNAbp domain that has a sequence that is at least 90%, at least 95%, at least 98%, or at least 99% identical to SpCas9-NRCH. In some embodiments, the disclosed base editors comprise a napDNAbp domain that comprises SpCas9-NRCH. An example of an NRCH PAM is CACC (5′-CACC-3′). The SpCas9-NRCH has an amino acid sequence as presented in SEQ ID NO: 436 (underligned residues are mutated relative to SpCas9):

(SEQ ID NO: 436)
MDKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEE
DKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQI
GDQYADLFLAAKNLSDAILLSDILRVNTEITKAPLSASMVKRYDEHHQDLTLLKALV
RQQLPEKYKEIFFDQSKNGYAGYIDGGASQEEFYKFIKPILEKMDGTEELLVKLNRED
LLRKQRTFDNGIIPHQIHLGELHAILRRQGDFYPFLKDNREKIEKILTFRIPYYVGPLAR
GNSRFAWMTRKSEETITPWNFEEVVDKGASAQSFIERMTNFDKNLPNEKVLPKHSLL
YEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDRE
MIEERLKTYAHLFDDKVMKQLKRLRYTGWGRLSRKLINGIRDKQSGKTILDFLKSDG
FANRNFMQLIHDDSLTFKEDIQKAQVSGQGDSLHEHIANLAGSPAIKKGILQTVKVV
DELVKVMGGHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVR
KVLSMPQVNIVKKTEVQTGGFSKESILPKGNSDKLIARKKDWDPKKYGGFNSPTVA
YSVLVVAKVEKGKSKKLKSVKELLGITIMERSSFEKNPIDFLEAKGYKEVKKDLIIKL
PKYSLFELENGRKRMLASAGVLQKGNELALPSKYVNFLYLASHYEKLKGSPEDNEQ
KQLFVEQHKHYLDEIIEQISEFSKRVILADANLDKVLSAYNKHRDKPIREQAENIIHLF
TLTNLGAPAAFKYFDTTINRKQYNTTKEVLDATLIRQSITGLYETRIDLSQLGGD

In some embodiments, the disclosed base editors comprise a napDNAbp domain that has a sequence that is at least 90%, at least 95%, at least 98%, or at least 99% identical to SpCas9-NRTH. In some embodiments, the disclosed base editors comprise a napDNAbp domain that comprises SpCas9-NRTH. The SpCas9-NRTH has an amino acid sequence as presented in SEQ ID NO: 437 (underligned residues are mutated relative to SpCas9):

(SEQ ID NO: 437)
MDKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEE
DKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQI
GDQYADLFLAAKNLSDAILLSDILRVNTEITKAPLSASMVKRYDEHHQDLTLLKALV
RQQLPEKYKEIFFDQSKNGYAGYIDGGASQEEFYKFIKPILEKMDGTEELLVKLNRED
LLRKQRTFDNGIIPHQIHLGELHAILRRQGDFYPFLKDNREKIEKILTFRIPYYVGPLAR
GNSRFAWMTRKSEETITPWNFEEVVDKGASAQSFIERMTNFDKNLPNEKVLPKHSLL
YEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDRE
MIEERLKTYAHLFDDKVMKQLKRLRYTGWGRLSRKLINGIRDKQSGKTILDFLKSDG
FANRNFMQLIHDDSLTFKEDIQKAQVSGQGDSLHEHIANLAGSPAIKKGILQTVKVV
DELVKVMGGHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVR
KVLSMPQVNIVKKTEVQTGGFSKESILPKGNSDKLIARKKDWDPKKYGGFNSPTVA
YSVLVVAKVEKGKSKKLKSVKELLGITIMERSSFEKNPIGFLEAKGYKEVKKDLIIKL
PKYSLFELENGRKRMLASASVLHKGNELALPSKYVNFLYLASHYEKLKGSSEDNKQ
KQLFVEQHKHYLDEIIEQISEFSKRVILADANLDKVLSAYNKHRDKPIREQAENIIHLF
TLTNLGASAAFKYFDTTIGRKLYTSTKEVLDATLIHQSITGLYETRIDLSQLGGD

In other embodiments, the napDNAbp of any of the disclosed base editors comprises a Cas9 derived from a Streptococcus macacae, e.g., Streptococcus macacae NCTC 11558, or SmacCas9, or a variant thereof. In some embodiments, the napDNAbp comprises a hybrid variant of SmacCas9 that incorporates an SpCas9 domain with the SmacCas9 domain and is known as Spy-macCas9, or a variant thereof. In some embodiments, the napDNAbp comprises a hybrid variant of SmacCas9 that incorporates an increased nucleolytic variant of an SpCas9 (iSpy Cas9) domain and is known as iSpy-macCas9. Relative to Spymac-Cas9, iSpyMac-Cas9 contains two mutations, R221K and N394K, that were identified by deep mutational scans of Spy Cas9 that raise modification rates of the protein on most targets. See Jakimo et al., bioRxiv, A Cas9 with Complete PAM Recognition for Adenine Dinucleotides (September 2018), herein incorporated by reference. Jakimo et al. showed that the hybrids Spy-macCas9 and iSpy-macCas9 recognize a short 5′-NAA-3′ PAM and recognized all evaluated adenine dinucleotide PAM sequences and posseseds robust editing efficiency in human cells. Liu et al. engineered base editors containing Spy-mac Cas9, and demonstrated that cytidine and adenine base editors containing Spymac domains can induce efficient C-to-T and A-to-G conversions in vivo. In addition, Liu et al. suggested that the PAM scope of Spy-mac Cas9 may be 5′-TAAA-3′, rather than 5′-NAA-3′ as reported by Jakimo et al. See Liu et al. Cell Discovery (2019) 5:58, herein incorporated by reference.

In some embodiments, the disclosed base editors comprise a napDNAbp domain that has a sequence that is at least 90%, at least 95%, at least 98%, or at least 99% identical to iSpyMac-Cas9. In some embodiments, the disclosed base editors comprise a napDNAbp domain that comprises iSpyMac-Cas9. The iSpyMac-Cas9 has an amino acid sequence as presented in SEQ ID NO: 439 (R221K and N394K mutations are underlined):

(SEQ ID NO: 439)
DKKYSIGLDIGTNSVGWAVITDEYKVPSKKFKVLGNTDRHSIKKNLIGALL
FDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGH
FLIEGDLNPDNSDVDKLFIQLVQTYNQLFEENPINASGVDAKAILSARLSKSRKLENLI
AQLPGEKKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLLKALVRQ
QLPEKYKEIFFDQSKNGYAGYIDGGASQEEFYKFIKPILEKMDGTEELLVKLKREDLL
RKQRTFDNGSIPHQIHLGELHAILRRQEDFYPFLKDNREKIEKILTFRIPYYVGPLARG
NSRFAWMTRKSEETITPWNFEEVVDKGASAQSFIERMTNFDKNLPNEKVLPKHSLLY
EYFTVYNELTKVKYVTEGMRKPAFLSGEQKKAIVDLLFKTNRKVTVKQLKEDYFKK
IECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSIDNKVLT
RSDKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRK
VLSMPQVNIVKKTEIQTVGQNGGLFDDNPKSPLEVTPSKLVPLKKELNPKKYGGYQK
PTTAYPVLLITDTKQLIPISVMNKKQFEQNPVKFLRDRGYQQVGKNDFIKLPKYTLVD
IGDGIKRLWASSKEIHKGNQLVVSKKSQILLYHAHHLDSDLSNDYLQNHNQQFDVLF
NEIISFSKKCKLGKEHIQKIENVYSNKKNSASIEELAESFIKLLGFTQLGATSPFNFLGV
KLNQKQYKGKKDYILPCTEGTLIRQSITGLYETRVDLSKIGE

In other embodiments, the napDNAbp of any of the disclosed base editors is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piezophila Argunaute (MpAgo) protein. The CRISPR-associated Marinitoga piezophila Argunaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other argonaute proteins may be used, and are within the scope of this disclosure.

In some embodiments, the napDNAbp is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

It will also be appreciated that Cas9 enzymes from different bacterial species (i.e., Cas9 orthologs) can have varying PAM specificities. For example, Cas9 from Staphylococcus aureus (SaCas9) recognizes NGRRT (SEQ ID NO: 201) or NGRRN (SEQ ID NO: 202). In addition, Cas9 from Neisseria meningitis (NmeCas and Nme2Cas9) recognizes NNNNGATT (SEQ ID NO: 203). A Cas9 from Staphylococcus auricularis (SauriCas9) recognizes NNGG (SEQ ID NO: 204) and NNNGG (SEQ ID NO: 205). A Cas9 from Streptococcus thermophilis (StCas9) recognizes NNAGAAW (SEQ ID NO: 206). A Cas9 from Treponema denticola (TdCas) recognizes NAAAAC (SEQ ID NO: 207). The compact Cas9 ortholog from derived from Campylobacter jejuni (CjCas9) recognizes recognizes NNNNACA (SEQ ID NO: 208) and NNNNACAC (SEQ ID NO: 209) PAMs. These are example are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference).

In some embodiments, the disclosed base editors comprise a napDNAbp domain comprising a SpCas9-NG, which has a PAM that corresponds to NGN. In some embodiments, the disclosed base editors comprise a napDNAbp domain that has a sequence that is at least 90%, at least 95%, at least 98%, or at least 99% identical to SpCas9-NG. The sequence of SpCas9-NG is illustrated below:

(SEQ ID NO: 210)
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESIRPKRNSDKLIARKKDWDPKKYGGFVSPTVAYSVLVV
AKVEKGKSKKLKSVKELLGITIMERSSFEKNPIDFLEAKGYKEVKKDLIIKLPKYSLFE
LENGRKRMLASARFLQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PRAFKYFDTTIDRKVYRSTKEVLDATLIHQSITGLYETRIDLSQLGGD

In some embodiments, the disclosed base editors comprise a napDNAbp domain comprising a SpCas9n-NG (or nCas9-NG), which has a PAM that corresponds to NGN. In some embodiments, the disclosed base editors comprise a napDNAbp domain that has a sequence that is at least 90%, at least 95%, at least 98%, or at least 99% identical to an nCas9-NG. In some embodiments, the disclosed base editors comprise a napDNAbp domain comprising a high fidelity SpCas9n-NG (or HF-nCas9-NG), which has a PAM that corresponds to NGN. In some embodiments, the disclosed base editors comprise a napDNAbp domain that has a sequence that is at least 90%, at least 95%, at least 98%, or at least 99% identical to an HF-nCas9-NG.

In some embodiments, the disclosed base editors comprise a napDNAbp domain comprising a S. aureus Cas9 nickase KKH, or SaCas9-KKH, which has a PAM that corresponds to NNNRRT (SEQ ID NO: 211). This Cas9 variant contains the amino acid substitutions D10A, E782K, N968K, and R1015H relative to wild-type SaCas9, set forth as SEQ ID NO: 377. In some embodiments, the disclosed base editors comprise a napDNAbp domain that has a sequence that is at least 90%, at least 95%, at least 98%, or at least 99% identical to SaCas9-KKH. The sequence of SaCas9-KKH is illustrated below:

(SEQ ID NO: 212)
MGKRNYILGLAIGITSVGYGIIDYETRDVIDAGVRLFKEANVENNEGRRSKRGARRL
KRRRRHRIQRVKKLLFDYNLLTDHSELSGINPYEARVKGLSQKLSEEEFSAALLHLAK
RRGVHNVNEVEEDTGNELSTKEQISRNSKALEEKYVAELQLERLKKDGEVRGSINRF
KTSDYVKEAKQLLKVQKAYHQLDQSFIDTYIDLLETRRTYYEGPGEGSPFGWKDIKE
WYEMLMGHCTYFPEELRSVKYAYNADLYNALNDLNNLVITRDENEKLEYYEKFQII
ENVFKQKKKPTLKQIAKEILVNEEDIKGYRVTSTGKPEFTNLKVYHDIKDITARKEIIE
NAELLDQIAKILTIYQSSEDIQEELTNLNSELTQEEIEQISNLKGYTGTHNLSLKAINLIL
DELWHTNDNQIAIFNRLKLVPKKVDLSQQKEIPTTLVDDFILSPVVKRSFIQSIKVINAI
IKKYGLPNDIIIELAREKNSKDAQKMINEMQKRNRQTNERIEEIIRTTGKENAKYLIEKI
KLHDMQEGKCLYSLEAIPLEDLLNNPFNYEVDHIIPRSVSFDNSFNNKVLVKQEENSK
KGNRTPFQYLSSSDSKISYETFKKHILNLAKGKGRISKTKKEYLLEERDINRFSVQKDF
INRNLVDTRYATRGLMNLLRSYFRVNNLDVKVKSINGGFTSFLRRKWKFKKERNKG
YKHHAEDALIIANADFIFKEWKKLDKAKKVMENQMFEEKQAESMPEIETEQEYKEIF
ITPHQIKHIKDFKDYKYSHRVDKKPNRKLINDTLYSTRKDDKGNTLIVNNLNGLYDK
DNDKLKKLINKSPEKLLMYHHDPQTYQKLKLIMEQYGDEKNPLYKYYEETGNYLTK
YSKKDNGPVIKKIKYYGNKLNAHLDITDDYPNSRNKVVKLSLKPYRFDVYLDNGVY
KFVTVKNLDVIKKENYYEVNSKCYEEAKKLKKISNQAEFIASFYKNDLIKINGELYRV
IGVNNDLLNRIEVNMIDITYREYLENMNDKRPPHIIKTIASKTQSIKKYSTDILGNLYE
VKSKKHPQIIKKG

In some embodiments, the disclosed base editors comprise a napDNAbp domain comprising a a S. pyogenes Cas9 nickase KKH, or SpCas9-KKH, which has a PAM that corresponds to NNNRRT (SEQ ID NO: 213).

In some embodiments, the disclosed base editors comprise a napDNAbp domain comprising a xCas9, an evolved variant of SpCas9. In some embodiments, the disclosed base editors comprise a napDNAbp domain that has a sequence that is at least 90%, at least 95%, at least 98%, or at least 99% identical to xCas9. The sequence of xCas9 is illustrated below:

(SEQ ID NO: 214)
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDTKLQLSKDTYDDDLDNLLAQIGDQYA
DLFLAAKNLSDAILLSDILRVNTEITKAPLSASMIKLYDEHHQDLTLLKALVRQQLPE
KYKEIFFDQSKNGYAGYIDGGASQEEFYKFIKPILEKMDGTEELLVKLNREDLLRKQR
TFDNGIIPHQIHLGELHAILRRQEDFYPFLKDNREKIEKILTFRIPYYVGPLARGNSRFA
WMTRKSEETITPWNFEKVVDKGASAQSFIERMTNFDKNLPNEKVLPKHSLLYEYFTV
YNELTKVKYVTEGMRKPAFLSGDQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FIQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKN
RGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLFEL
ENGRKRMLASAGVLQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGD

### Cas9 Circular Permutants

In various embodiments, the base editors disclosed herein may comprise a circular permutant of Cas9.

The term “circularly permuted Cas9” or “circular permutant” of Cas9 or “CP-Cas9”) refers to any Cas9 protein, or variant thereof, that occurs or has been modify to engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, and Huang, T. P. et al. Circularly permuted and PAM-modified Cas9 variants broaden the targeting scope of base editors. Nat. Biotechnol. 37, 626-631 (2019). each of are incorporated herein by reference. Reference is also made to International Publication No. WO 2020/041751, published Feb. 27, 2020, herein incorporated by reference. The present disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or naturally occurring Cas9 or equivalent thereof, may be reconfigured as a circular permutant variant.

In various embodiments, the circular permutants of Cas9 may have the following structure:
N-terminus-[original C-terminus]-[optional linker]-[original N-terminus]-C-terminus.

As an example, the present disclosure contemplates the following circular permutants of canonical S. pyogenes Cas9 (1368 amino acids of UniProtKB-Q99ZW2 (CAS9_STRP1) (numbering is based on the amino acid position in SEQ ID NO: 326)):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus; or
N-terminus-[10-1368]-[optional linker]-[1-9]-C-terminus, or the corresponding circularpermutants of other Cas9 proteins (including other Cas9 orthologs, variants, etc).

In particular embodiments, the circular permuant Cas9 has the following structure (based on S. pyogenes Cas9 (1368 amino acids of UniProtKB-Q99ZW2 (CAS9_STRP1) (numbering is based on the amino acid position in SEQ ID NO: 326):
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In still other embodiments, the circular permuant Cas9 has the following structure (based on S. pyogenes Cas9 (1368 amino acids of UniProtKB-Q99ZW2 (CAS9_STRP1) (numbering is based on the amino acid position in SEQ ID NO: 326):
N-terminus-[103-1368]-[optional linker]-[1-102]-C-terminus;
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc.).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the C-terminal 95% or more of the amino acids of a Cas9 (e.g., amino acids about 1300-1368), or the C-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., any one of SEQ ID NOs: 18-25). The N-terminal portion may correspond to the N-terminal 95% or more of the amino acids of a Cas9 (e.g., amino acids about 1-1300), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., of SEQ ID NO: 326).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus, includes or corresponds to the C-terminal 30% or less of the amino acids of a Cas9 (e.g., amino acids 1012-1368 of SEQ ID NO: 326). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., the Cas9 of SEQ ID NO: 326). In some embodiments, the C-terminal fragment that is rearranged to the N-terminus, includes or corresponds to the C-terminal 410 residues or less of a Cas9 (e.g., the Cas9 of SEQ ID NO: 326). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., the Cas9 of SEQ ID NO: 326). In some embodiments, the C-terminal portion that is rearranged to the N-terminus, includes or corresponds to the C-terminal 357, 341, 328, 120, or 69 residues of a Cas9 (e.g., the Cas9 of SEQ ID NO: 326).

In other embodiments, circular permutant Cas9 variants may be defined as a topological rearrangement of a Cas9 primary structure based on the following method, which is based on S. pyogenes Cas9 of SEQ ID NO: 326: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to preceed the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 326) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 326, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

Exemplary CP-Cas9 amino acid sequences, based on the Cas9 of SEQ ID NO: 326, are provided below in which linker sequences are indicated by underlining and optional methionine (M) residues are indicated in bold. It should be appreciated that the disclosure provides CP-Cas9 sequences that do not include a linker sequence or that include different linker sequences. It should be appreciated that CP-Cas9 sequences may be based on Cas9 sequences other than that of SEQ ID NO: 326 and any examples provided herein are not meant to be limiting. Exemplary CP-Cas9 sequences are as follows:

| CP name | Sequence | SEQ ID NO: |
|---|---|---|
| CP1012 | DYKVYDVRKMIAKSEQEIGKATAKYFFYSNIMNFFKTEITLA | SEQ ID NO: |
| | NGEIRKRPLIETNGETGEIVWDKGRDFATVRKVLSMPQVNIV | 396 |
| | KKTEVQTGGFSKESILPKRNSDKLIARKKDWDPKKYGGFDS | |
| | PTVAYSVLVVAKVEKGKSKKLKSVKELLGITIMERSSFEKNPI | |
| | DFLEAKGYKEVKKDLIIKLPKYSLFELENGRKRMLASAGEL | |
| | QKGNELALPSKYVNFLYLASHYEKLKGSPEDNEQKQLFVEQ | |
| | HKHYLDEIIEQISEFSKRVILADANLDKVLSAYNKHRDKPIRE | |
| | QAENIIHLFTLTNLGAPAAFKYFDTTIDRKRYTSTKEVLDATL | |
| | IHQSITGLYETRIDLSQLGGDGGSGGSGGSGGSGGSGGSGGD | |
| | KKYSIGLAIGTNSVGWAVITDEYKVPSKKFKVLGNTDRHSIK | |
| | KNLIGALLFDSGETAEATRLKRTARRRYTRRKNRICYLQEIFS | |
| | NEMAKVDDSFFHRLEESFLVEEDKKHERHPIFGNIVDEVAYH | |
| | EKYPTIYHLRKKLVDSTDKADLRLIYLALAHMIKFRGHFLIE | |
| | GDLNPDNSDVDKLFIQLVQTYNQLFEENPINASGVDAKAILS | |
| | ARLSKSRRLENLIAQLPGEKKNGLFGNLIALSLGLTPNFKSNF | |
| | DLAEDAKLQLSKDTYDDDLDNLLAQIGDQYADLFLAAKNL | |
| | SDAILLSDILRVNTEITKAPLSASMIKRYDEHHQDLTLLKALV | |
| | RQQLPEKYKEIFFDQSKNGYAGYIDGGASQEEFYKFIKPILEK | |
| | MDGTEELLVKLNREDLLRKQRTFDNGSIPHQIHLGELHAILR | |
| | RQEDFYPFLKDNREKIEKILTFRIPYYVGPLARGNSRFAWMT | |
| | RKSEETITPWNFEEVVDKGASAQSFIERMTNFDKNLPNEKV | |
| | LPKHSLLYEYFTVYNELTKVKYVTEGMRKPAFLSGEQKKAI | |
| | VDLLFKTNRKVTVKQLKEDYFKKIECFDSVEISGVEDRFNAS | |
| | LGTYHDLLKIIKDKDFLDNEENEDILEDIVLTLTLFEDREMIE | |
| | ERLKTYAHLFDDKVMKQLKRRRYTGWGRLSRKLINGIRDK | |
| | QSGKTILDFLKSDGFANRNFMQLIHDDSLTFKEDIQKAQVSG | |
| | QGDSLHEHIANLAGSPAIKKGILQTVKVVDELVKVMGRHKP | |
| | ENIVIEMARENQTTQKGQKNSRERMKRIEEGIKELGSQILKE | |
| | HPVENTQLQNEKLYLYYLQNGRDMYVDQELDINRLSDYDV | |
| | DHIVPQSFLKDDSIDNKVLTRSDKNRGKSDNVPSEEVVKKM | |
| | KNYWRQLLNAKLITQRKFDNLTKAERGGLSELDKAGFIKRQ | |
| | LVETRQITKHVAQILDSRMNTKYDENDKLIREVKVITLKSKL | |
| | VSDFRKDFQFYKVREINNYHHAHDAYLNAVVGTALIKKYPK | |
| | LESEFVYG | |
| CP1028 | EIGKATAKYFFYSNIMNFFKTEITLANGEIRKRPLIETNGETG | SEQ ID NO: |
| | EIVWDKGRDFATVRKVLSMPQVNIVKKTEVQTGGFSKESILP | 397 |
| | KRNSDKLIARKKDWDPKKYGGFDSPTVAYSVLVVAKVEKG | |
| | KSKKLKSVKELLGITIMERSSFEKNPIDFLEAKGYKEVKKDLI | |
| | IKLPKYSLFELENGRKRMLASAGELQKGNELALPSKYVNFL | |
| | YLASHYEKLKGSPEDNEQKQLFVEQHKHYLDEIIEQISEFSK | |
| | RVILADANLDKVLSAYNKHRDKPIREQAENIIHLFTLTNLGA | |
| | PAAFKYFDTTIDRKRYTSTKEVLDATLIHQSITGLYETRIDLS | |
| | QLGGDGGSGGSGGSGGSGGSGGSGGMDKKYSIGLAIGTNS | |
| | VGWAVITDEYKVPSKKFKVLGNTDRHSIKKNLIGALLFDSGE | |
| | TAEATRLKRTARRRYTRRKNRICYLQEIFSNEMAKVDDSFFH | |
| | RLEESFLVEEDKKHERHPIFGNIVDEVAYHEKYPTIYHLRKKL | |
| | VDSTDKADLRLIYLALAHMIKFRGHFLIEGDLNPDNSDVDK | |
| | LFIQLVQTYNQLFEENPINASGVDAKAILSARLSKSRRLENLI | |
| | AQLPGEKKNGLFGNLIALSLGLTPNFKSNFDLAEDAKLQLSK | |
| | DTYDDDLDNLLAQIGDQYADLFLAAKNLSDAILLSDILRVNT | |
| | EITKAPLSASMIKRYDEHHQDLTLLKALVRQQLPEKYKEIFF | |
| | DQSKNGYAGYIDGGASQEEFYKFIKPILEKMDGTEELLVKLN | |
| | REDLLRKQRTFDNGSIPHQIHLGELHAILRRQEDFYPFLKDN | |
| | REKIEKILTFRIPYYVGPLARGNSRFAWMTRKSEETITPWNFE | |
| | EVVDKGASAQSFIERMTNFDKNLPNEKVLPKHSLLYEYFTV | |
| | YNELTKVKYVTEGMRKPAFLSGEQKKAIVDLLFKTNRKVTV | |
| | KQLKEDYFKKIECFDSVEISGVEDRFNASLGTYHDLLKIIKD | |
| | KDFLDNEENEDILEDIVLTLTLFEDREMIEERLKTYAHLFDDK | |
| | VMKQLKRRRYTGWGRLSRKLINGIRDKQSGKTILDFLKSDG | |
| | FANRNFMQLIHDDSLTFKEDIQKAQVSGQGDSLHEHIANLA | |
| | GSPAIKKGILQTVKVVDELVKVMGRHKPENIVIEMARENQT | |
| | TQKGQKNSRERMKRIEEGIKELGSQILKEHPVENTQLQNEKL | |
| | YLYYLQNGRDMYVDQELDINRLSDYDVDHIVPQSFLKDDSI | |
| | DNKVLTRSDKNRGKSDNVPSEEVVKKMKNYWRQLLNAKLI | |
| | TQRKFDNLTKAERGGLSELDKAGFIKRQLVETRQITKHVAQI | |
| | LDSRMNTKYDENDKLIREVKVITLKSKLVSDFRKDFQFYKV | |
| | REINNYHHAHDAYLNAVVGTALIKKYPKLESEFVYGDYKVY | |
| | DVRKMIAKSEQ | |
| CP1041 | NIMNFFKTEITLANGEIRKRPLIETNGETGEIVWDKGRDFATV | SEQ ID NO: |
| | RKVLSMPQVNIVKKTEVQTGGFSKESILPKRNSDKLIARKKD | 398 |
| | WDPKKYGGFDSPTVAYSVLVVAKVEKGKSKKLKSVKELLGI | |
| | TIMERSSFEKNPIDFLEAKGYKEVKKDLIIKLPKYSLFELENG | |
| | RKRMLASAGELQKGNELALPSKYVNFLYLASHYEKLKGSPE | |
| | DNEQKQLFVEQHKHYLDEIIEQISEFSKRVILADANLDKVLS | |
| | AYNKHRDKPIREQAENIIHLFTLTNLGAPAAFKYFDTTIDRKR | |
| | YTSTKEVLDATLIHQSITGLYETRIDLSQLGGDGGSGGSGGS | |
| | GGSGGSGGSGGDKKYSIGLAIGTNSVGWAVITDEYKVPSKK | |
| | FKVLGNTDRHSIKKNLIGALLFDSGETAEATRLKRTARRRYT | |
| | RRKNRICYLQEIFSNEMAKVDDSFFHRLEESFLVEEDKKHER | |
| | HPIFGNIVDEVAYHEKYPTIYHLRKKLVDSTDKADLRLIYLA | |
| | LAHMIKFRGHFLIEGDLNPDNSDVDKLFIQLVQTYNQLFEEN | |
| | PINASGVDAKAILSARLSKSRRLENLIAQLPGEKKNGLFGNLI | |
| | ALSLGLTPNFKSNFDLAEDAKLQLSKDTYDDDLDNLLAQIG | |
| | DQYADLFLAAKNLSDAILLSDILRVNTEITKAPLSASMIKRYD | |
| | EHHQDLTLLKALVRQQLPEKYKEIFFDQSKNGYAGYIDGGA | |
| | SQEEFYKFIKPILEKMDGTEELLVKLNREDLLRKQRTFDNGSI | |
| | PHQIHLGELHAILRRQEDFYPFLKDNREKIEKILTFRIPYYVGP | |
| | LARGNSRFAWMTRKSEETITPWNFEEVVDKGASAQSFIERM | |
| | TNFDKNLPNEKVLPKHSLLYEYFTVYNELTKVKYVTEGMR | |
| | KPAFLSGEQKKAIVDLLFKTNRKVTVKQLKEDYFKKIECFDS | |
| | VEISGVEDRFNASLGTYHDLLKIIKDKDFLDNEENEDILEDIV | |
| | LTLTLFEDREMIEERLKTYAHLFDDKVMKQLKRRRYTGWGR | |
| | LSRKLINGIRDKQSGKTILDFLKSDGFANRNFMQLIHDDSLTF | |
| | KEDIQKAQVSGQGDSLHEHIANLAGSPAIKKGILQTVKVVDE | |
| | LVKVMGRHKPENIVIEMARENQTTQKGQKNSRERMKRIEEG | |
| | IKELGSQILKEHPVENTQLQNEKLYLYYLQNGRDMYVDQEL | |
| | DINRLSDYDVDHIVPQSFLKDDSIDNKVLTRSDKNRGKSDN | |
| | VPSEEVVKKMKNYWRQLLNAKLITQRKFDNLTKAERGGLS | |
| | ELDKAGFIKRQLVETRQITKHVAQILDSRMNTKYDENDKLIR | |
| | EVKVITLKSKLVSDFRKDFQFYKVREINNYHHAHDAYLNAV | |
| | VGTALIKKYPKLESEFVYGDYKVYDVRKMIAKSEQEIGKAT | |
| | AKYFFYS | |
| CP1249 | PEDNEQKQLFVEQHKHYLDEIIEQISEFSKRVILADANLDKV | SEQ ID NO: |
| | LSAYNKHRDKPIREQAENIIHLFTLTNLGAPAAFKYFDTTIDR | 399 |
| | KRYTSTKEVLDATLIHQSITGLYETRIDLSQLGGDGGSGGSG | |
| | GSGGSGGSGGSGGMDKKYSIGLAIGTNSVGWAVITDEYKVP | |
| | SKKFKVLGNTDRHSIKKNLIGALLFDSGETAEATRLKRTARR | |
| | RYTRRKNRICYLQEIFSNEMAKVDDSFFHRLEESFLVEEDKK | |
| | HERHPIFGNIVDEVAYHEKYPTIYHLRKKLVDSTDKADLRLI | |
| | YLALAHMIKFRGHFLIEGDLNPDNSDVDKLFIQLVQTYNQLF | |
| | EENPINASGVDAKAILSARLSKSRRLENLIAQLPGEKKNGLF | |
| | GNLIALSLGLTPNFKSNFDLAEDAKLQLSKDTYDDDLDNLL | |
| | AQIGDQYADLFLAAKNLSDAILLSDILRVNTEITKAPLSASMI | |
| | KRYDEHHQDLTLLKALVRQQLPEKYKEIFFDQSKNGYAGYI | |
| | DGGASQEEFYKFIKPILEKMDGTEELLVKLNREDLLRKQRTF | |
| | DNGSIPHQIHLGELHAILRRQEDFYPFLKDNREKIEKILTFRIP | |
| | YYVGPLARGNSRFAWMTRKSEETITPWNFEEVVDKGASAQ | |
| | SFIERMTNFDKNLPNEKVLPKHSLLYEYFTVYNELTKVKYV | |
| | TEGMRKPAFLSGEQKKAIVDLLFKTNRKVTVKQLKEDYFKK | |
| | IECFDSVEISGVEDRFNASLGTYHDLLKIIKDKDFLDNEENED | |
| | ILEDIVLTLTLFEDREMIEERLKTYAHLFDDKVMKQLKRRRY | |
| | TGWGRLSRKLINGIRDKQSGKTILDFLKSDGFANRNFMQLIH | |
| | DDSLTFKEDIQKAQVSGQGDSLHEHIANLAGSPAIKKGILQT | |
| | VKVVDELVKVMGRHKPENIVIEMARENQTTQKGQKNSRER | |
| | MKRIEEGIKELGSQILKEHPVENTQLQNEKLYLYYLQNGRD | |
| | MYVDQELDINRLSDYDVDHIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTKY | |
| | DENDKLIREVKVITLKSKLVSDFRKDFQFYKVREINNYHHA | |
| | HDAYLNAVVGTALIKKYPKLESEFVYGDYKVYDVRKMIAK | |
| | SEQEIGKATAKYFFYSNIMNFFKTEITLANGEIRKRPLIETNGE | |
| | TGEIVWDKGRDFATVRKVLSMPQVNIVKKTEVQTGGFSKES | |
| | ILPKRNSDKLIARKKDWDPKKYGGFDSPTVAYSVLVVAKVE | |
| | KGKSKKLKSVKELLGITIMERSSFEKNPIDFLEAKGYKEVKK | |
| | DLIIKLPKYSLFELENGRKRMLASAGELQKGNELALPSKYV | |
| | NFLYLASHYEKLKGS | |
| CP1300 | KPIREQAENIIHLFTLTNLGAPAAFKYFDTTIDRKRYTSTKEV | SEQ ID NO: |
| | LDATLIHQSITGLYETRIDLSQLGGDGGSGGSGGSGGSGGSG | 400 |
| | GSGGDKKYSIGLAIGTNSVGWAVITDEYKVPSKKFKVLGNT | |
| | DRHSIKKNLIGALLFDSGETAEATRLKRTARRRYTRRKNRIC | |
| | YLQEIFSNEMAKVDDSFFHRLEESFLVEEDKKHERHPIFGNIV | |
| | DEVAYHEKYPTIYHLRKKLVDSTDKADLRLIYLALAHMIKFR | |
| | GHFLIEGDLNPDNSDVDKLFIQLVQTYNQLFEENPINASGVD | |
| | AKAILSARLSKSRRLENLIAQLPGEKKNGLFGNLIALSLGLTP | |
| | NFKSNFDLAEDAKLQLSKDTYDDDLDNLLAQIGDQYADLFL | |
| | AAKNLSDAILLSDILRVNTEITKAPLSASMIKRYDEHHQDLTL | |
| | LKALVRQQLPEKYKEIFFDQSKNGYAGYIDGGASQEEFYKFI | |
| | KPILEKMDGTEELLVKLNREDLLRKQRTFDNGSIPHQIHLGE | |
| | LHAILRRQEDFYPFLKDNREKIEKILTFRIPYYVGPLARGNSR | |
| | FAWMTRKSEETITPWNFEEVVDKGASAQSFIERMTNFDKNL | |
| | PNEKVLPKHSLLYEYFTVYNELTKVKYVTEGMRKPAFLSGE | |
| | QKKAIVDLLFKTNRKVTVKQLKEDYFKKIECFDSVEISGVED | |
| | RFNASLGTYHDLLKIIKDKDFLDNEENEDILEDIVLTLTLFED | |
| | REMIEERLKTYAHLFDDKVMKQLKRRRYTGWGRLSRKLIN | |
| | GIRDKQSGKTILDFLKSDGFANRNFMQLIHDDSLTFKEDIQK | |
| | AQVSGQGDSLHEHIANLAGSPAIKKGILQTVKVVDELVKVM | |
| | GRHKPENIVIEMARENQTTQKGQKNSRERMKRIEEGIKELGS | |
| | QILKEHPVENTQLQNEKLYLYYLQNGRDMYVDQELDINRLS | |
| | DYDVDHIVPQSFLKDDSIDNKVLTRSDKNRGKSDNVPSEEV | |
| | VKKMKNYWRQLLNAKLITQRKFDNLTKAERGGLSELDKAG | |
| | FIKRQLVETRQITKHVAQILDSRMNTKYDENDKLIREVKVIT | |
| | LKSKLVSDFRKDFQFYKVREINNYHHAHDAYLNAVVGTALI | |
| | KKYPKLESEFVYGDYKVYDVRKMIAKSEQEIGKATAKYFFY | |
| | SNIMNFFKTEITLANGEIRKRPLIETNGETGEIVWDKGRDFAT | |
| | VRKVLSMPQVNIVKKTEVQTGGFSKESILPKRNSDKLIARKK | |
| | DWDPKKYGGFDSPTVAYSVLVVAKVEKGKSKKLKSVKELL | |
| | GITIMERSSFEKNPIDFLEAKGYKEVKKDLIIKLPKYSLFELE | |
| | NGRKRMLASAGELQKGNELALPSKYVNFLYLASHYEKLKG | |
| | SPEDNEQKQLFVEQHKHYLDEIIEQISEFSKRVILADANLDK | |
| | VLSAYNKHRD | |

The Cas9 circular permutants that may be useful in the base editor constructs described herein. Exemplary C-terminal fragments of Cas9, based on the Cas9 of SEQ ID NO: 326, which may be rearranged to an N-terminus of Cas9, are provided below. It should be appreciated that such C-terminal fragments of Cas9 are exemplary and are not meant to be limiting. These exemplary CP-Cas9 fragments have the following sequences:

| CP name | Sequence | SEQ ID NO: |
|---|---|---|
| CP1012 C- | DYKVYDVRKMIAKSEQEIGKATAKYFFYSNIMNFFKTEITLA | SEQ ID NO: |
| terminal | NGEIRKRPLIETNGETGEIVWDKGRDFATVRKVLSMPQVNIV | 401 |
| fragment | KKTEVQTGGFSKESILPKRNSDKLIARKKDWDPKKYGGFDS | |
| | PTVAYSVLVVAKVEKGKSKKLKSVKELLGITIMERSSFEKNPI | |
| | DFLEAKGYKEVKKDLIIKLPKYSLFELENGRKRMLASAGEL | |
| | QKGNELALPSKYVNFLYLASHYEKLKGSPEDNEQKQLFVEQ | |
| | HKHYLDEIIEQISEFSKRVILADANLDKVLSAYNKHRDKPIRE | |
| | QAENIIHLFTLTNLGAPAAFKYFDTTIDRKRYTSTKEVLDATL | |
| | IHQSITGLYETRIDLSQLGGD | |
| CP1028 C- | EIGKATAKYFFYSNIMNFFKTEITLANGEIRKRPLIETNGETG | SEQ ID NO: |
| terminal | EIVWDKGRDFATVRKVLSMPQVNIVKKTEVQTGGFSKESILP | 402 |
| fragment | KRNSDKLIARKKDWDPKKYGGFDSPTVAYSVLVVAKVEKG | |
| | KSKKLKSVKELLGITIMERSSFEKNPIDFLEAKGYKEVKKDLI | |
| | IKLPKYSLFELENGRKRMLASAGELQKGNELALPSKYVNFL | |
| | YLASHYEKLKGSPEDNEQKQLFVEQHKHYLDEIIEQISEFSK | |
| | RVILADANLDKVLSAYNKHRDKPIREQAENIIHLFTLTNLGA | |
| | PAAFKYFDTTIDRKRYTSTKEVLDATLIHQSITGLYETRIDLS | |
| | QLGGD | |
| CP1041 C- | NIMNFFKTEITLANGEIRKRPLIETNGETGEIVWDKGRDFATV | SEQ ID NO: |
| terminal | RKVLSMPQVNIVKKTEVQTGGFSKESILPKRNSDKLIARKKD | 403 |
| fragment | WDPKKYGGFDSPTVAYSVLVVAKVEKGKSKKLKSVKELLGI | |
| | TIMERSSFEKNPIDFLEAKGYKEVKKDLIIKLPKYSLFELENG | |
| | RKRMLASAGELQKGNELALPSKYVNFLYLASHYEKLKGSPE | |
| | DNEQKQLFVEQHKHYLDEIIEQISEFSKRVILADANLDKVLS | |
| | AYNKHRDKPIREQAENIIHLFTLTNLGAPAAFKYFDTTIDRKR | |
| | YTSTKEVLDATLIHQSITGLYETRIDLSQLGGD | |
| CP1249 C- | PEDNEQKQLFVEQHKHYLDEIIEQISEFSKRVILADANLDKV | SEQ ID NO: |
| terminal | LSAYNKHRDKPIREQAENIIHLFTLTNLGAPAAFKYFDTTIDR | 404 |
| fragment | KRYTSTKEVLDATLIHQSITGLYETRIDLSQLGGD | |
| CP1300 C- | KPIREQAENIIHLFTLTNLGAPAAFKYFDTTIDRKRYTSTKEV | SEQ ID NO: |
| terminal | LDATLIHQSITGLYETRIDLSQLGGD | 405 |
| fragment | | |

In some embodiments, the napDNAbp domain comprises a combination of more than one Cas homolog or variant, such as a circularly permuted Cas variant. In some embodiments, the napDNAbp domain comprises a first Cas variant and a second Cas variant. In some embodiments, the napDNAbp domain comprises a first Cas variant comprising a Cas9-NG and a second Cas variant comprising a Cas9-CP1041 variant. The combination of the CP1041 variant and the NG variant enables both broadened PAM targeting and an expanded editing window. Such a domain is referred to herein as “SpCas9-NG-CP1041.” In some embodiments, the napDNAbp domain comprises an amino acid sequence that has at least 80%, at least 8%, at least 90%, at least 92.5%, at least 95%, at least 97.5%, at least 98%, or at least 99% sequence identity to SEQ ID NO: 463. In some embodiments, the napDNAbp domain comprises the sequence of SEQ ID NO: 463.

(SEQ ID NO: 463)
NIMNFFKTEITLANGEIRKRPLIETNGETGEIVWDKGRDFATVRKVLSMPQVNIVKKT
EVQTGGFSKESIRPKRNSDKLIARKKDWDPKKYGGFVSPTVAYSVLVVAKVEKGKS
KKLKSVKELLGITIMERSSFEKNPIDFLEAKGYKEVKKDLIIKLPKYSLFELENGRKRM
LASARFLQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPRAFKYFDT
TIDRKVYRSTKEVLDATLIHQSITGLYETRIDLSQLGGDGGSGGSGGSGGSGGSGGSG
GDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRT
FDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYS

In some embodiments, the napDNAbp domain comprises a first Cas variant comprising a Cas9-VRQR and a second Cas variant comprising a Cas9-CP1041 variant. Such a domain is referred to herein as “SpCas9-NG-VRQR.” In some embodiments, the napDNAbp domain comprises an amino acid sequence that has at least 80%, at least 8%, at least 90%, at least 92.5%, at least 95%, at least 97.5%, at least 98%, or at least 99% sequence identity to SEQ ID NO: 464. In some embodiments, the napDNAbp domain comprises the sequence of SEQ ID NO: 464.

(SEQ ID NO: 464)
NIMNFFKTEITLANGEIRKRPLIETNGETGEIVWDKGRDFATVRKVLSMPQ
VNIVKKTEVQTGGFSKESIRPKRNSDKLIARKKDWDPKKYGGFVSPTVAYSVLVVA
KVEKGKSKKLKSVKELLGITIMERSSFEKNPIDFLEAKGYKEVKKDLIIKLPKYSLFEL
ENGRKRMLASARFLQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAP
RAFKYFDTTIDRKVYRSTKEVLDATLIHQSITGLYETRIDLSQLGGDGGSGGSGGSGG
SGGSGGSGGDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDR
EMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSE
QEIGKATAKYFFYS

High Fidelity Cas9 Domains and Variants Thereof that Display Higher Specificity

Some aspects of the disclosure provide high fidelity Cas9 (HFCas9) domains of the fusion proteins provided herein. In some embodiments, high fidelity Cas9 domains are engineered Cas9 domains comprising one or more mutations that decrease electrostatic interactions between the Cas9 domain and the sugar-phosphate backbone of DNA, as compared to a corresponding wild-type Cas9 domain. Without wishing to be bound by any particular theory, high fidelity Cas9 domains that have decreased electrostatic interactions with the sugar-phosphate backbone of DNA may have less off-target effects. In some embodiments, the Cas9 domain (e.g., a wild type Cas9 domain) comprises one or more mutations that decrease the association between the Cas9 domain and the sugar-phosphate backbone of DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, or more.

In some embodiments, any of the Cas9 fusion proteins provided herein comprise one or more of N497X, R661X, Q695X, and/or Q926X mutation of the amino acid sequence provided in SEQ ID NO: 6, or corresponding mutation(s) in any Cas9 provided herein, such as any of the amino acid sequences provided in SEQ ID NOs: 4-26, wherein X is any amino acid. In some embodiments, any of the Cas9 fusion proteins provided herein comprise one or more of N497A, R661A, Q695A, and/or Q926A mutation of the amino acid sequence provided in SEQ ID NO: 6, or a corresponding mutation in any Cas9 provided herein, such as any of the amino acid sequences provided in SEQ ID NOs: 4-26. In some embodiments, any of the Cas9 fusion proteins provided herein comprise one or more of D10A, N497A, R661A, Q695A, and/or Q926A mutation of the amino acid sequence provided in SEQ ID NO: 6, such as any of the amino acid sequences provided in SEQ ID NOs: 4-26. In some embodiments, the Cas9 domain (e.g., of any of the fusion proteins provided herein) comprises the amino acid sequence as set forth in SEQ ID NO: 20. In some embodiments, the Cas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to SEQ ID NO: 20. Cas9 domains with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference.

It should be appreciated that any of the base editors (or fusion proteins) provided herein, for example, any of the C to G base editors provided herein, may be converted into high fidelity base editors by modifying the Cas9 domain as described herein to generate high fidelity base editors, for example, a high fidelity C to G base editor. In some embodiments, the high fidelity Cas9 domain is a dCas9 domain. In some embodiments, the high fidelity Cas9 domain is a nCas9 domain (HF-nCas9) (i.e., HF1, SEQ ID NO: 20).

In some embodiments, the napDNAbp domain of any of the disclosed CGBEs is a Hypa-Cas9 domain. The Hypa-Cas9 domain contains N692A, M694A, Q695A, D1135E mutations in the amino acid sequence provided in SEQ ID NO: 6 (SEQ ID NO: 727), or a corresponding mutation in any Cas9 provided herein, such as any of the amino acid sequences provided in SEQ ID NOs: 4-26. Hypa-Cas9 is described in further detail in Ikeda et al., Communications Biology Vol. 2: 371 (2019) and Chen, J. S. et al., Nature 550, 407-410 (2017), each of which is incorporated bu reference herein. HypaCas9 demonstrates a high ratio of on-target to off-target cleavage activity. The Hypa-nCas9 domain contains D10A, N692A, M694A, Q695A, D1135E mutations relative to the amino acid sequence provided in SEQ ID NO: 6 (SEQ ID NO: 728), or a corresponding mutation in any Cas9 provided herein, such as any of the amino acid sequences provided in SEQ ID NOs: 4-26.

In some embodiments, the napDNAbp domain of any of the disclosed CGBEs contains a combination of substitutions from high fidelity Cas9 HF1 and from HypaCas9, or an HF-Hypa-Cas9 domain. In some embodiments, the napDNAbp domain is nickase domain that is an HF-Hypa-Cas9 nickase domain (SEQ ID NO: 731), which contains the D10A, N692A, M694A, Q695A, D1135E mutations relative to the amino acid sequence provided in SEQ ID NO: 6, or a corresponding mutation in any Cas9 provided herein, such as any of the amino acid sequences provided in SEQ ID NOs: 4-26.

In some embodiments, the napDNAbp domain of any of the disclosed CGBEs is an e-Cas9 domain, such as an e-SpCas9 domain, or e-SpCas9(1.1) (SEQ ID NO: 726). The e-Cas9 domain contains K848A, K1003A, and R1060A mutations in the amino acid sequence provided in SEQ ID NO: 6, or a corresponding mutation in any Cas9 provided herein, such as any of the amino acid sequences provided in SEQ ID NOs: 4-26. e-Cas9 is described in further detail in Anzalone, Koblan & Liu, Nature Biotechnology Vol. 38, 824-844 (2020), which is incorporated by reference herein. e-SpCas9(1.1) was discovered through alanine scanning of positively charged residues that line the non-target-strand binding groove, with the hypothesis that interrupting interactions between these residues and the negatively charged nucleic acid backbone would decrease binding affinity. After screening mutants, the combination of K848A, K1003A and R1060A mutations was chosen, and the resulting e-SpCas9(1.1) variant displayed efficient and precise genome editing in human cells. The e-Cas9 variant may also be provided as a nickase. The e-Cas9n domain contains D10A, K848A, K1003A, and R1060A mutations in the amino acid sequence provided in SEQ ID NO: 6, or a corresponding mutation in any Cas9 provided herein, such as any of the amino acid sequences provided in SEQ ID NOs: 4-26.

An enhanced fidelity variant has been engineered combining mutations found in e-SpCas9(1.1) and SpCas9-HF1 (see Kulcsir, P. I. et al. Genome Biol. 18, 190 (2017)). Accordingly, in some embodiments, the napDNAbp domain is Cas9 variant containing a combination of substitutions from e-Cas9 and HypaCas9, or an e-Hypa-Cas9 domain (or HeFSpCas9 domain). The e-Hypa-SpCas9 domain (SEQ ID NO: 730) contains K848A, K1003A, R1060A, N692A, M694A, Q695A, and D1135E substitutions in SEQ ID NO: 6. The e-Hypa-nCas9 domain contains D10A, K848A, K1003A, R1060A, N692A, M694A, Q695A, and D1135E substitutions in SEQ ID NO: 6. In some embodiments, the napDNAbp domain is an e-Cas9 combined with a HF-nCas9, i.e., an e-HF-nCas9 domain, such as the e-HF-SpCas9n domain of SEQ ID NO: 729. In some embodiments, the napDNAbp domain is an e-Cas9 combined with a HF-Hypa-nCas9, or an e-HF-Hypa-nCas9 domain. The e-Hypa-HF-SpCas9n domain (SEQ ID NO: 732) contains D10A, K848A, K1003A, R1060A, N497A, R661A, Q695A, Q926A, N692A, M694A, Q695A, and D1135E substitutions in SEQ ID NO: 6.

It will be appreciated that all of of the disclosed Cas9 variants for use in the napDNAbp domains of the provided CGBEs can be engineered to have nickase activity (e.g., to contain a D10A substitution) or can be engineered to be nuclease-inactive (e.g., to contain D10A and H840A substitutions).

In some embodiments, the napDNAbp domain of any of the disclosed CGBEs comprises an amino acid sequence that is at least 85%, 90%, 92.5%, 95%, 97%, 98%, or 99% identical to any of the sequences set forth as SEQ ID NOs: 20 and 726-727, 729-732. In some embodiments, the napDNAbp domain of any of the disclosed CGBEs is selected from SEQ ID NOs: 20 and 726-727, 729-732.

The High Fidelity Cas9 nickase domain (HF-nCas9), where mutations relative to Cas9 of SEQ ID NO: 6 are shown in bold and underline:

(SEQ ID NO: 20)
DKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTAFDKNLPNEKVLPKHSLLYEYFTVY
NELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERL
KTYAHLFDDKVMKQLKRRRYTGWGALSRKLINGIRDKQSGKTILDFLKSDGFANRN
FMALIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRAITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD

### Other Cas9 Equivalents

In some embodiments, the base editors described herein can include any Cas9 equivalent. As used herein, the term “Cas9 equivalent” is a broad term that encompasses any napDNAbp protein that serves the same function as Cas9 in the present base editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The base editors described here embrace any Cas9 equivalent that would provide the same or similar function as Cas9 despite that the Cas9 equivalent may be based on a protein that arose through convergent evolution.

For example, CasX is a Cas9 equivalent that reportedly has the same function as Cas9 but which evolved through convergent evolution. Thus, the CasX protein described in Liu et al., “CasX enzymes comprises a distinct family of RNA-guided genome editors,” Nature, 2019, Vol. 566: 218-223, is contemplated to be used with the base editors described herein. In addition, any variant or modification of CasX is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), CasX, CasY, Cpf1, C2c1, C2c2, C2C3, Argonaute, Cas12a, and Cas12b. One example of a nucleic acid programmable DNA-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN (SEQ ID NO: 215), or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference. The state of the art may also now refer to Cpf1 enzymes as Cas12a.

In still other embodiments, the Cas protein may include any CRISPR associated protein, including but not limited to, Cas12a, Cas12b, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type SpCas9 polypeptide of SEQ ID NO: 326).

In various other embodiments, the napDNAbp domain may be any of the following proteins: a Cas9, a Cpf1, a CasX, a CasY, a C2c1, a C2c2, a C2c3, a GeoCas9, a CjCas9, a Cas12a, a Cas12b, a Cas12g, a Cas12h, a Cas12i, a Cas13a, a Cas13b, a Cas13c, a Cas13d, a Cas14 (Cas12f), a Csn2, an xCas9, an SpCas9-NG, an nCas9-NG, a high-fidelity Cas9 (HFCas9), a HypaCas9, an e-Cas9, an e-HypaCas9, a HF-nCas9, a HF-nCas9-NG, a Sniper-nCas9, an HF-Hypa-nCas9, an e-HF-Hypa-nCas9, a circularly permuted Cas9 domain such as CP1012, CP1028, CP1041, CP1249, and CP1300, or an Argonaute (Ago) domain, a Cas9-KKH, a SmacCas9, a Spy-macCas9, an SpCas9-VRQR, an SpCas9-VRER, an SpCas9-VQR, an SpCas9-EQR, an SpCas9-NRRH, an SpaCas9-NRTH, an SpCas9-NRCH. In some embodiments, the napDNAbp domain may be any of the following proteins: an LbCas12a, an AsCas12a, a CeCas12a, an MbCas12a, a Cas(D (Cas12j), an SpCas9-NG-CP1041, an SpCas9-NG-VRQR, a CasMINI, a Cas7-11, an NmeCas9, an Nme2Cas9, a SauriCas9, an StCas9, a TdCas9, a SuperFi-Cas9, or a variant thereof.

In some embodiments, the napDNAbp domain is selected from an nCas9, an nCas9-NG, an HF-Cas9, a HypaCas9, a HF-nCas9, a HF-nCas9-NG, an HF-Hypa-nCas9, an e-HF-Hypa-nCas9, and an e-HypaCas9. In particular embodiments, the napDNAbp domain is an HF-nCas9, a HF-nCas9-NG, Hypa-nCas9, or an HF-Hypa-nCas9.

In certain embodiments, the base editors contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery. The canonical SpCas9 protein is 1368 amino acids in length and has a predicted molecular weight of 158 kilodaltons. The term “small-sized Cas9 variant”, as used herein, refers to any Cas9 variant-naturally occurring, engineered, or otherwise-that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein.

In various embodiments, the base editors disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein. Exemplary small-sized Cas9 variants include, but are not limited to, SaCas9 and LbCas12a.

In some embodiments, the napDNAbp domain of any of the disclosed base editors comprises an LbCas12a, such as a wild-type LbCas12a. In some embodiments, the napDNAbp domain of any of the disclosed based editors is comprises at least 80%, at least 85%, at least 90%, at least 95%, or at least 99% sequence identity to SEQ ID NO: 381. In some embodiments, the napDNAbp domain of any of the disclosed base editors comprises the amino acid sequence of SEQ ID NO: 381.

In some embodiments, the napDNAbp domain of any of the disclosed base editors comprises an AsCas12a, such as a wild-type AsCas12a. In some embodiments, the napDNAbp domain of any of the disclosed base editors comprises a mutant AsCas12a, such as an engineered AsCas12a, or enAsCas12a. In some embodiments, the napDNAbp domain of any of the disclosed based editors is comprises at least 80%, at least 85%, at least 90%, at least 95%, or at least 99% sequence identity to SEQ ID NO: 383. In some embodiments, the napDNAbp domain of any of the disclosed base editors comprises the amino acid sequence of SEQ ID NO: 383.

| Description | Sequence | SEQ ID NO: |
|---|---|---|
| SaCas9 | MGKRNYILGLDIGITSVGYGIIDYETRDVIDAGVRLFKEANVEN | SEQ ID NO: |
| Staphylococcuss | NEGRRSKRGARRLKRRRRHRIQRVKKLLFDYNLLTDHSELSGI | 377 |
| aureus | NPYEARVKGLSQKLSEEEFSAALLHLAKRRGVHNVNEVEEDT | |
| 1053 AA | GNELSTKEQISRNSKALEEKYVAELQLERLKKDGEVRGSINRF | |
| 123 kDa | KTSDYVKEAKQLLKVQKAYHQLDQSFIDTYIDLLETRRTYYEG | |
| | PGEGSPFGWKDIKEWYEMLMGHCTYFPEELRSVKYAYNADLY | |
| | NALNDLNNLVITRDENEKLEYYEKFQIIENVFKQKKKPTLKQIA | |
| | KEILVNEEDIKGYRVTSTGKPEFTNLKVYHDIKDITARKEIIENA | |
| | ELLDQIAKILTIYQSSEDIQEELTNLNSELTQEEIEQISNLKGYTG | |
| | THNLSLKAINLILDELWHTNDNQIAIFNRLKLVPKKVDLSQQKE | |
| | IPTTLVDDFILSPVVKRSFIQSIKVINAIIKKYGLPNDIIIELAREK | |
| | NSKDAQKMINEMQKRNRQTNERIEEIIRTTGKENAKYLIEKIKL | |
| | HDMQEGKCLYSLEAIPLEDLLNNPFNYEVDHIIPRSVSFDNSFN | |
| | NKVLVKQEENSKKGNRTPFQYLSSSDSKISYETFKKHILNLAK | |
| | GKGRISKTKKEYLLEERDINRFSVQKDFINRNLVDTRYATRGL | |
| | MNLLRSYFRVNNLDVKVKSINGGFTSFLRRKWKFKKERNKGY | |
| | KHHAEDALIIANADFIFKEWKKLDKAKKVMENQMFEEKQAES | |
| | MPEIETEQEYKEIFITPHQIKHIKDFKDYKYSHRVDKKPNRKLIN | |
| | DTLYSTRKDDKGNTLIVNNLNGLYDKDNDKLKKLINKSPEKLL | |
| | MYHHDPQTYQKLKLIMEQYGDEKNPLYKYYEETGNYLTKYS | |
| | KKDNGPVIKKIKYYGNKLNAHLDITDDYPNSRNKVVKLSLKP | |
| | YRFDVYLDNGVYKFVTVKNLDVIKKENYYEVNSKCYEEAKK | |
| | LKKISNQAEFIASFYKNDLIKINGELYRVIGVNNDLLNRIEVNMI | |
| | DITYREYLENMNDKRPPHIIKTIASKTQSIKKYSTDILGNLYEVK | |
| | SKKHPQIIKK | |
| NmeCas9 | MAAFKPNSINYILGLDIGIASVGWAMVEIDEEENPIRLIDLGVR | SEQ ID NO: |
| N. meningitidis | VFERAEVPKTGDSLAMARRLARSVRRLTRRRAHRLLRTRRLL | 378 |
| 1083 AA | KREGVLQAANFDENGLIKSLPNTPWQLRAAALDRKLTPLEWS | |
| 124.5 kDa | AVLLHLIKHRGYLSQRKNEGETADKELGALLKGVAGNAHALQ | |
| | TGDFRTPAELALNKFEKESGHIRNQRSDYSHTFSRKDLQAELIL | |
| | LFEKQKEFGNPHVSGGLKEGIETLLMTQRPALSGDAVQKMLG | |
| | HCTFEPAEPKAAKNTYTAERFIWLTKLNNLRILEQGSERPLTDT | |
| | ERATLMDEPYRKSKLTYAQARKLLGLEDTAFFKGLRYGKDNA | |
| | EASTLMEMKAYHAISRALEKEGLKDKKSPLNLSPELQDEIGTA | |
| | FSLFKTDEDITGRLKDRIQPEILEALLKHISFDKFVQISLKALRRI | |
| | VPLMEQGKRYDEACAEIYGDHYGKKNTEEKIYLPPIPADEIRNP | |
| | VVLRALSQARKVINGVVRRYGSPARIHIETAREVGKSFKDRKEI | |
| | EKRQEENRKDREKAAAKFREYFPNFVGEPKSKDILKLRLYEQQ | |
| | HGKCLYSGKEINLGRLNEKGYVEIDAALPFSRTWDDSFNNKVL | |
| | VLGSENQNKGNQTPYEYFNGKDNSREWQEFKARVETSRFPRS | |
| | KKQRILLQKFDEDGFKERNLNDTRYVNRFLCQFVADRMRLTG | |
| | KGKKRVFASNGQITNLLRGFWGLRKVRAENDRHHALDAVVV | |
| | ACSTVAMQQKITRFVRYKEMNAFDGKTIDKETGEVLHQKTHF | |
| | PQPWEFFAQEVMIRVFGKPDGKPEFEEADTLEKLRTLLAEKLSS | |
| | RPEAVHEYVTPLFVSRAPNRKMSGQGHMETVKSAKRLDEGVS | |
| | VLRVPLTQLKLKDLEKMVNREREPKLYEALKARLEAHKDDPA | |
| | KAFAEPFYKYDKAGNRTQQVKAVRVEQVQKTGVWVRNHNGI | |
| | ADNATMVRVDVFEKGDKYYLVPIYSWQVAKGILPDRAVVQGK | |
| | DEEDWQLIDDSFNFKFSLHPNDLVEVITKKARMFGYFASCHRG | |
| | TGNINIRIHDLDHKIGKNGILEGIGVKTALSFQKYQIDELGKEIR | |
| | PCRLKKRPPVR | |
| CjCas9 | MARILAFDIGISSIGWAFSENDELKDCGVRIFTKVENPKTGESL | SEQ ID NO: |
| C. jejuni | ALPRRLARSARKRLARRKARLNHLKHLIANEFKLNYEDYQSF | 379 |
| 984 AA | DESLAKAYKGSLISPYELRFRALNELLSKQDFARVILHIAKRRG | |
| 114.9 kDa | YDDIKNSDDKEKGAILKAIKQNEEKLANYQSVGEYLYKEYFQ | |
| | KFKENSKEFTNVRNKKESYERCIAQSFLKDELKLIFKKQREFGF | |
| | SFSKKFEEEVLSVAFYKRALKDFSHLVGNCSFFTDEKRAPKNSP | |
| | LAFMFVALTRIINLLNNLKNTEGILYTKDDLNALLNEVLKNGTL | |
| | TYKQTKKLLGLSDDYEFKGEKGTYFIEFKKYKEFIKALGEHNL | |
| | SQDDLNEIAKDITLIKDEIKLKKALAKYDLNQNQIDSLSKLEFK | |
| | DHLNISFKALKLVTPLMLEGKKYDEACNELNLKVAINEDKKDF | |
| | LPAFNETYYKDEVTNPVVLRAIKEYRKVLNALLKKYGKVHKI | |
| | NIELAREVGKNHSQRAKIEKEQNENYKAKKDAELECEKLGLKI | |
| | NSKNILKLRLFKEQKEFCAYSGEKIKISDLQDEKMLEIDHIYPYS | |
| | RSFDDSYMNKVLVFTKQNQEKLNQTPFEAFGNDSAKWQKIEV | |
| | LAKNLPTKKQKRILDKNYKDKEQKNFKDRNLNDTRYIARLVL | |
| | NYTKDYLDFLPLSDDENTKLNDTQKGSKVHVEAKSGMLTSAL | |
| | RHTWGFSAKDRNNHLHHAIDAVIIAYANNSIVKAFSDFKKEQE | |
| | SNSAELYAKKISELDYKNKRKFFEPFSGFRQKVLDKIDEIFVSKP | |
| | ERKKPSGALHEETFRKEEEFYQSYGGKEGVLKALELGKIRKVN | |
| | GKIVKNGDMFRVDIFKHKKTNKFYAVPIYTMDFALKVLPNKAV | |
| | ARSKKGEIKDWILMDENYEFCFSLYKDSLILIQTKDMQEPEFV | |
| | YYNAFTSSTVSLIVSKHDNKFETLSKNQKILFKNANEKEVIAKS | |
| | IGIQNLKVFEKYIVSALGEVTKAEFRQREDFKK | |
| GeoCas9 | MRYKIGLDIGITSVGWAVMNLDIPRIEDLGVRIFDRAENPQTGE | SEQ ID NO: |
| G. | SLALPRRLARSARRRLRRRKHRLERIRRLVIREGILTKEELDKLF | 380 |
| stearothermophilus | EEKHEIDVWQLRVEALDRKLNNDELARVLLHLAKRRGFKSNR | |
| 1087 AA | KSERSNKENSTMLKHIEENRAILSSYRTVGEMIVKDPKFALHK | |
| 127 kDa | RNKGENYTNTIARDDLEREIRLIFSKQREFGNMSCTEEFENEYI | |
| | TIWASQRPVASKDDIEKKVGFCTFEPKEKRAPKATYTFQSFIAW | |
| | EHINKLRLISPSGARGLTDEERRLLYEQAFQKNKITYHDIRTLLH | |
| | LPDDTYFKGIVYDRGESRKQNENIRFLELDAYHQIRKAVDKVY | |
| | GKGKSSSFLPIDFDTFGYALTLFKDDADIHSYLRNEYEQNGKR | |
| | MPNLANKVYDNELIEELLNLSFTKFGHLSLKALRSILPYMEQG | |
| | EVYSSACERAGYTFTGPKKKQKTMLLPNIPPIANPVVMRALTQ | |
| | ARKVVNAIIKKYGSPVSIHIELARDLSQTFDERRKTKKEQDENR | |
| | KKNETAIRQLMEYGLTLNPTGHDIVKFKLWSEQNGRCAYSLQP | |
| | IEIERLLEPGYVEVDHVIPYSRSLDDSYTNKVLVLTRENREKGN | |
| | RIPAEYLGVGTERWQQFETFVLINKQFSKKKRDRLLRLHYDEN | |
| | EETEFKNRNLNDTRYISRFFANFIREHLKFAESDDKQKVYTVN | |
| | GRVTAHLRSRWEFNKNREESDLHHAVDAVIVACTTPSDIAKVT | |
| | AFYQRREQNKELAKKTEPHFPQPWPHFADELRARLSKHPKESI | |
| | KALNLGNYDDQKLESLQPVFVSRMPKRSVTGAAHQETLRRYV | |
| | GIDERSGKIQTVVKTKLSEIKLDASGHFPMYGKESDPRTYEAIR | |
| | QRLLEHNNDPKKAFQEPLYKPKKNGEPGPVIRTVKIIDTKNQVI | |
| | PLNDGKTVAYNSNIVRVDVFEKDGKYYCVPVYTMDIMKGILP | |
| | NKAIEPNKPYSEWKEMTEDYTFRFSLYPNDLIRIELPREKTVKT | |
| | AAGEEINVKDVFVYYKTIDSANGGLELISHDHRFSLRGVGSRT | |
| | LKRFEKYQVDVLGNIYKVRGEKRVGLASSAHSKPGKTIRPLQS | |
| | TRD | |
| LbCas12a | MSKLEKFTNCYSLSKTLRFKAIPVGKTQENIDNKRLLVEDEKR | SEQ ID NO: |
| L. bacterium | AEDYKGVKKLLDRYYLSFINDVLHSIKLKNLNNYISLFRKKTR | 381 |
| 1228 AA | TEKENKELENLEINLRKEIAKAFKGNEGYKSLFKKDIIETILPEF | |
| 143.9 kDa | LDDKDEIALVNSFNGFTTAFTGFFDNRENMFSEEAKSTSIAFRCI | |
| | NENLTRYISNMDIFEKVDAIFDKHEVQEIKEKILNSDYDVEDFF | |
| | EGEFFNFVLTQEGIDVYNAIIGGFVTESGEKIKGLNEYINLYNQ | |
| | KTKQKLPKFKPLYKQVLSDRESLSFYGEGYTSDEEVLEVFRNT | |
| | LNKNSEIFSSIKKLEKLFKNFDEYSSAGIFVKNGPAISTISKDIFG | |
| | EWNVIRDKWNAEYDDIHLKKKAVVTEKYEDDRRKSFKKIGSF | |
| | SLEQLQEYADADLSVVEKLKEIIIQKVDEIYKVYGSSEKLFDAD | |
| | FVLEKSLKKNDAVVAIMKDLLDSVKSFENYIKAFFGEGKETNR | |
| | DESFYGDFVLAYDILLKVDHIYDAIRNYVTQKPYSKDKFKLYF | |
| | QNPQFMGGWDKDKETDYRATILRYGSKYYLAIMDKKYAKCL | |
| | QKIDKDDVNGNYEKINYKLLPGPNKMLPKVFFSKKWMAYYN | |
| | PSEDIQKIYKNGTFKKGDMFNLNDCHKLIDFFKDSISRYPKWS | |
| | NAYDFNFSETEKYKDIAGFYREVEEQGYKVSFESASKKEVDKL | |
| | VEEGKLYMFQIYNKDFSDKSHGTPNLHTMYFKLLFDENNHGQ | |
| | IRLSGGAELFMRRASLKKEELVVHPANSPIANKNPDNPKKTTTL | |
| | SYDVYKDKRFSEDQYELHIPIAINKCPKNIFKINTEVRVLLKHD | |
| | DNPYVIGIDRGERNLLYIVVVDGKGNIVEQYSLNEIINNENGIRI | |
| | KTDYHSLLDKKEKERFEARQNWTSIENIKELKAGYISQVVHKI | |
| | CELVEKYDAVIALEDLNSGFKNSRVKVEKQVYQKFEKMLIDKL | |
| | NYMVDKKSNPCATGGALKGYQITNKFESFKSMSTQNGFIFYIP | |
| | AWLTSKIDPSTGFVNLLKTKYTSIADSKKFISSFDRIMYVPEEDL | |
| | FEFALDYKNFSRTDADYIKKWKLYSYGNRIRIFRNPKKNNVFD | |
| | WEEVCLTSAYKELFNKYGINYQQGDIRALLCEQSDKAFYSSFM | |
| | ALMSLMLQMRNSITGRTDVDFLISPVKNSDGIFYDSRNYEAQE | |
| | NAILPKNADANGAYNIARKVLWAIGQFKKAEDEKLDKVKIAIS | |
| | NKEWLEYAQTSVKH | |
| BhCas12b | MATRSFILKIEPNEEVKKGLWKTHEVLNHGIAYYMNILKLIRQE | SEQ ID NO: |
| B. hisashii | AIYEHHEQDPKNPKKVSKAEIQAELWDFVLKMQKCNSFTHEV | 382 |
| 1108 AA | DKDEVFNILRELYEELVPSSVEKKGEANQLSNKFLYPLVDPNSQ | |
| 130.4 kDa | SGKGTASSGRKPRWYNLKIAGDPSWEEEKKKWEEDKKKDPL | |
| | AKILGKLAEYGLIPLFIPYTDSNEPIVKEIKWMEKSRNQSVRRL | |
| | DKDMFIQALERFLSWESWNLKVKEEYEKVEKEYKTLEERIKE | |
| | DIQALKALEQYEKERQEQLLRDTLNTNEYRLSKRGLRGWREII | |
| | QKWLKMDENEPSEKYLEVFKDYQRKHPREAGDYSVYEFLSK | |
| | KENHFIWRNHPEYPYLYATFCEIDKKKKDAKQQATFTLADPIN | |
| | HPLWVRFEERSGSNLNKYRILTEQLHTEKLKKKLTVQLDRLIYP | |
| | TESGGWEEKGKVDIVLLPSRQFYNQIFLDIEEKGKHAFTYKDE | |
| | SIKFPLKGTLGGARVQFDRDHLRRYPHKVESGNVGRIYFNMTV | |
| | NIEPTESPVSKSLKIHRDDFPKVVNFKPKELTEWIKDSKGKKLK | |
| | SGIESLEIGLRVMSIDLGQRQAAAASIFEVVDQKPDIEGKLFFPI | |
| | KGTELYAVHRASFNIKLPGETLVKSREVLRKAREDNLKLMNQK | |
| | LNFLRNVLHFQQFEDITEREKRVTKWISRQENSDVPLVYQDELI | |
| | QIRELMYKPYKDWVAFLKQLHKRLEVEIGKEVKHWRKSLSDG | |
| | RKGLYGISLKNIDEIDRTRKFLLRWSLRPTEPGEVRRLEPGQRF | |
| | AIDQLNHLNALKEDRLKKMANTIIMHALGYCYDVRKKKWQA | |
| | KNPACQIILFEDLSNYNPYEERSRFENSKLMKWSRREIPRQVAL | |
| | QGEIYGLQVGEVGAQFSSRFHAKTGSPGIRCSVVTKEKLQDNR | |
| | FFKNLQREGRLTLDKIAVLKEGDLYPDKGGEKFISLSKDRKCVT | |
| | THADINAAQNLQKRFWTRTHGFYKVYCKAYQVDGQTVYIPES | |
| | KDQKQKIIEEFGEGYFILKDGVYEWVNAGKLKIKKGSSKQSSS | |
| | ELVDSDILKDSFDLASELKGEKLMLYRDPSGNVFPSDKWMAA | |
| | GVFFGKLERILISKLTNQYSISTIEDDSSKQSM | |

Additional exemplary Cas9 equivalent protein sequences can include the following:

| Description | Sequence |
|---|---|
| AsCas12a | MTQFEGFTNLYQVSKTLRFELIPQGKTLKHIQEQGFIEEDKARNDHYKELKPII |
| (previously | DRIYKTYADQCLQLVQLDWENLSAAIDSYRKEKTEETRNALIEEQATYRNAIH |
| known as | DYFIGRTDNLTDAINKRHAEIYKGLFKAELFNGKVLKQLGTVTTTEHENALLR |
| Cpf1) | SFDKFTTYFSGFYENRKNVFSAEDISTAIPHRIVQDNFPKFKENCHIFTRLITAV |
| Acidaminococcus | PSLREHFENVKKAIGIFVSTSIEEVFSFPFYNQLLTQTQIDLYNQLLGGISREAG |
| sp. (strain | TEKIKGLNEVLNLAIQKNDETAHIIASLPHRFIPLFKQILSDRNTLSFILEEFKSD |
| BV3L6) | EEVIQSFCKYKTLLRNENVLETAEALFNELNSIDLTHIFISHKKLETISSALCDH |
| UniProtKB | WDTLRNALYERRISELTGKITKSAKEKVQRSLKHEDINLQEIISAAGKELSEAF |
| U2UMQ6 | KQKTSEILSHAHAALDQPLPTTLKKQEEKEILKSQLDSLLGLYHLLDWFAVDE |
| | SNEVDPEFSARLTGIKLEMEPSLSFYNKARNYATKKPYSVEKFKLNFQMPTLA |
| | SGWDVNKEKNNGAILFVKNGLYYLGIMPKQKGRYKALSFEPTEKTSEGFDK |
| | MYYDYFPDAAKMIPKCSTQLKAVTAHFQTHTTPILLSNNFIEPLEITKEIYDLN |
| | NPEKEPKKFQTAYAKKTGDQKGYREALCKWIDFTRDFLSKYTKTTSIDLSSLR |
| | PSSQYKDLGEYYAELNPLLYHISFQRIAEKEIMDAVETGKLYLFQIYNKDFAKG |
| | HHGKPNLHTLYWTGLFSPENLAKTSIKLNGQAELFYRPKSRMKRMAHRLGE |
| | KMLNKKLKDQKTPIPDTLYQELYDYVNHRLSHDLSDEARALLPNVITKEVSH |
| | EIIKDRRFTSDKFFFHVPITLNYQAANSPSKFNQRVNAYLKEHPETPIIGIDRGE |
| | RNLIYITVIDSTGKILEQRSLNTIQQFDYQKKLDNREKERVAARQAWSVVGTI |
| | KDLKQGYLSQVIHEIVDLMIHYQAVVVLENLNFGFKSKRTGIAEKAVYQQFE |
| | KMLIDKLNCLVLKDYPAEKVGGVLNPYQLTDQFTSFAKMGTQSGFLFYVPAP |
| | YTSKIDPLTGFVDPFVWKTIKNHESRKHFLEGFDFLHYDVKTGDFILHFKMNR |
| | NLSFQRGLPGFMPAWDIVFEKNETQFDAKGTPFIAGKRIVPVIENHRFTGRYR |
| | DLYPANELIALLEEKGIVFRDGSNILPKLLENDDSHAIDTMVALIRSVLQMRNS |
| | NAATGEDYINSPVRDLNGVCFDSRFQNPEWPMDADANGAYHIALKGQLLLN |
| | HLKESKDLKLQNGISNQDWLAYIQELRN (SEQ ID NO: 383) |
| AsCas12a | MTQFEGFTNLYQVSKTLRFELIPQGKTLKHIQEQGFIEEDKARNDHYKELKPII |
| nickase (e.g., | DRIYKTYADQCLQLVQLDWENLSAAIDSYRKEKTEETRNALIEEQATYRNAIH |
| R1226A) | DYFIGRTDNLTDAINKRHAEIYKGLFKAELFNGKVLKQLGTVTTTEHENALLR |
| | SFDKFTTYFSGFYENRKNVFSAEDISTAIPHRIVQDNFPKFKENCHIFTRLITAV |
| | PSLREHFENVKKAIGIFVSTSIEEVFSFPFYNQLLTQTQIDLYNQLLGGISREAG |
| | TEKIKGLNEVLNLAIQKNDETAHIIASLPHRFIPLFKQILSDRNTLSFILEEFKSD |
| | EEVIQSFCKYKTLLRNENVLETAEALFNELNSIDLTHIFISHKKLETISSALCDH |
| | WDTLRNALYERRISELTGKITKSAKEKVQRSLKHEDINLQEIISAAGKELSEAF |
| | KQKTSEILSHAHAALDQPLPTTLKKQEEKEILKSQLDSLLGLYHLLDWFAVDE |
| | SNEVDPEFSARLTGIKLEMEPSLSFYNKARNYATKKPYSVEKFKLNFQMPTLA |
| | SGWDVNKEKNNGAILFVKNGLYYLGIMPKQKGRYKALSFEPTEKTSEGFDK |
| | MYYDYFPDAAKMIPKCSTQLKAVTAHFQTHTTPILLSNNFIEPLEITKEIYDLN |
| | NPEKEPKKFQTAYAKKTGDQKGYREALCKWIDFTRDFLSKYTKTTSIDLSSLR |
| | PSSQYKDLGEYYAELNPLLYHISFQRIAEKEIMDAVETGKLYLFQIYNKDFAKG |
| | HHGKPNLHTLYWTGLFSPENLAKTSIKLNGQAELFYRPKSRMKRMAHRLGE |
| | KMLNKKLKDQKTPIPDTLYQELYDYVNHRLSHDLSDEARALLPNVITKEVSH |
| | EIIKDRRFTSDKFFFHVPITLNYQAANSPSKFNQRVNAYLKEHPETPIIGIDRGE |
| | RNLIYITVIDSTGKILEQRSLNTIQQFDYQKKLDNREKERVAARQAWSVVGTI |
| | KDLKQGYLSQVIHEIVDLMIHYQAVVVLENLNFGFKSKRTGIAEKAVYQQFE |
| | KMLIDKLNCLVLKDYPAEKVGGVLNPYQLTDQFTSFAKMGTQSGFLFYVPAP |
| | YTSKIDPLTGFVDPFVWKTIKNHESRKHFLEGFDFLHYDVKTGDFILHFKMNR |
| | NLSFQRGLPGFMPAWDIVFEKNETQFDAKGTPFIAGKRIVPVIENHRFTGRYR |
| | DLYPANELIALLEEKGIVFRDGSNILPKLLENDDSHAIDTMVALIRSVLQMANS |
| | NAATGEDYINSPVRDLNGVCFDSRFQNPEWPMDADANGAYHIALKGQLLLN |
| | HLKESKDLKLQNGISNQDWLAYIQELRN (SEQ ID NO: 384) |
| LbCas12a | MNYKTGLEDFIGKESLSKTLRNALIPTESTKIHMEEMGVIRDDELRAEKQQEL |
| (previously | KEIMDDYYRTFIEEKLGQIQGIQWNSLFQKMEETMEDISVRKDLDKIQNEKR |
| known as | KEICCYFTSDKRFKDLFNAKLITDILPNFIKDNKEYTEEEKAEKEQTRVLFQRF |
| Cpf1) | ATAFTNYFNQRRNNFSEDNISTAISFRIVNENSEIHLQNMRAFQRIEQQYPEEV |
| Lachnospiraceae | CGMEEEYKDMLQEWQMKHIYSVDFYDRELTQPGIEYYNGICGKINEHMNQF |
| bacterium | CQKNRINKNDFRMKKLHKQILCKKSSYYEIPFRFESDQEVYDALNEFIKTMK |
| GAM79 | KKEIIRRCVHLGQECDDYDLGKIYISSNKYEQISNALYGSWDTIRKCIKEEYM |
| Ref Seq. | DALPGKGEKKEEKAEAAAKKEEYRSIADIDKIISLYGSEMDRTISAKKCITEIC |
| WP_119623382.1 | DMAGQISIDPLVCNSDIKLLQNKEKTTEIKTILDSFLHVYQWGQTFIVSDIIEKD |
| | SYFYSELEDVLEDFEGITTLYNHVRSYVTQKPYSTVKFKLHFGSPTLANGWSQ |
| | SKEYDNNAILLMRDQKFYLGIFNVRNKPDKQIIKGHEKEEKGDYKKMIYNLL |
| | PGPSKMLPKVFITSRSGQETYKPSKHILDGYNEKRHIKSSPKFDLGYCWDLID |
| | YYKECIHKHPDWKNYDFHFSDTKDYEDISGFYREVEMQGYQIKWTYISADEI |
| | QKLDEKGQIFLFQIYNKDFSVHSTGKDNLHTMYLKNLFSEENLKDIVLKLNG |
| | EAELFFRKASIKTPIVHKKGSVLVNRSYTQTVGNKEIRVSIPEEYYTEIYNYLN |
| | HIGKGKLSSEAQRYLDEGKIKSFTATKDIVKNYRYCCDHYFLHLPITINFKAKS |
| | DVAVNERTLAYIAKKEDIHIIGIDRGERNLLYISVVDVHGNIREQRSFNIVNGY |
| | DYQQKLKDREKSRDAARKNWEEIEKIKELKEGYLSMVIHYIAQLVVKYNAV |
| | VAMEDLNYGFKTGRFKVERQVYQKFETMLIEKLHYLVFKDREVCEEGGVLR |
| | GYQLTYIPESLKKVGKQCGFIFYVPAGYTSKIDPTTGFVNLFSFKNLTNRESRQ |
| | DFVGKFDEIRYDRDKKMFEFSFDYNNYIKKGTILASTKWKVYTNGTRLKRIV |
| | VNGKYTSQSMEVELTDAMEKMLQRAGIEYHDGKDLKGQIVEKGIEAEIIDIFR |
| | LTVQMRNSRSESEDREYDRLISPVLNDKGEFFDTATADKTLPQDADANGAYCI |
| | ALKGLYEVKQIKENWKENEQFPRNKLVQDNKTWFDFMQKKRYL (SEQ ID |
| | NO: 385) |
| PcCas12a- | MAKNFEDFKRLYSLSKTLRFEAKPIGATLDNIVKSGLLDEDEHRAASYVKVK |
| previously | KLIDEYHKVFIDRVLDDGCLPLENKGNNNSLAEYYESYVSRAQDEDAKKKF |
| known at Cpf1 | KEIQQNLRSVIAKKLTEDKAYANLFGNKLIESYKDKEDKKKIIDSDLIQFINTAE |
| Prevotella | STQLDSMSQDEAKELVKEFWGFVTYFYGFFDNRKNMYTAEEKSTGIAYRLV |
| copri | NENLPKFIDNIEAFNRAITRPEIQENMGVLYSDFSEYLNVESIQEMFQLDYYN |
| Ref Seq. | MLLTQKQIDVYNAIIGGKTDDEHDVKIKGINEYINLYNQQHKDDKLPKLKAL |
| WP_119227726.1 | FKQILSDRNAISWLPEEFNSDQEVLNAIKDCYERLAENVLGDKVLKSLLGSLA |
| | DYSLDGIFIRNDLQLTDISQKMFGNWGVIQNAIMQNIKRVAPARKHKESEEDY |
| | EKRIAGIFKKADSFSISYINDCLNEADPNNAYFVENYFATFGAVNTPTMQRENL |
| | FALVQNAYTEVAALLHSDYPTVKHLAQDKANVSKIKALLDAIKSLQHFVKPL |
| | LGKGDESDKDERFYGELASLWAELDTVTPLYNMIRNYMTRKPYSQKKIKLNF |
| | ENPQLLGGWDANKEKDYATIILRRNGLYYLAIMDKDSRKLLGKAMPSDGEC |
| | YEKMVYKFFKDVTTMIPKCSTQLKDVQAYFKVNTDDYVLNSKAFNKPLTIT |
| | KEVFDLNNVLYGKYKKFQKGYLTATGDNVGYTHAVNVWIKFCMDFLNSYDS |
| | TCIYDFSSLKPESYLSLDAFYQDANLLLYKLSFARASVSYINQLVEEGKMYLF |
| | QIYNKDFSEYSKGTPNMHTLYWKALFDERNLADVVYKLNGQAEMFYRKKSI |
| | ENTHPTHPANHPILNKNKDNKKKESLFDYDLIKDRRYTVDKFMFHVPITMNF |
| | KSVGSENINQDVKAYLRHADDMHIIGIDRGERHLLYLVVIDLQGNIKEQYSLN |
| | EIVNEYNGNTYHTNYHDLLDVREEERLKARQSWQTIENIKELKEGYLSQVIH |
| | KITQLMVRYHAIVVLEDLSKGFMRSRQKVEKQVYQKFEKMLIDKLNYLVDK |
| | KTDVSTPGGLLNAYQLTCKSDSSQKLGKQSGFLFYIPAWNTSKIDPVTGFVNL |
| | LDTHSLNSKEKIKAFFSKFDAIRYNKDKKWFEFNLDYDKFGKKAEDTRTKWT |
| | LCTRGMRIDTFRNKEKNSQWDNQEVDLTTEMKSLLEHYYIDIHGNLKDAISA |
| | QTDKAFFTGLLHILKLTLQMRNSITGTETDYLVSPVADENGIFYDSRSCGNQLP |
| | ENADANGAYNIARKGLMLIEQIKNAEDLNNVKFDISNKAWLNFAQQKPYKN |
| | G (SEQ ID NO: 386) |
| ErCas12a- | MFSAKLISDILPEFVIHNNNYSASEKEEKTQVIKLESRFATSFKDYFKNRANCF |
| previously | SANDISSSSCHRIVNDNAEIFFSNALVYRRIVKNLSNDDINKISGDMKDSLKEM |
| known at Cpf1 | SLEEIYSYEKYGEFITQEGISFYNDICGKVNLFMNLYCQKNKENKNLYKLRKL |
| Eubacterium | HKQILCIADTSYEVPYKFESDEEVYQSVNGFLDNISSKHIVERLRKIGENYNG |
| rectale | YNLDKIYIVSKFYESVSQKTYRDWETINTALEIHYNNILPGNGKSKADKVKK |
| Ref Seq. | AVKNDLQKSITEINELVSNYKLCPDDNIKAETYIHEISHILNNFEAQELKYNPEI |
| WP_119223642.1 | HLVESELKASELKNVLDVIMNAFHWCSVFMTEELVDKDNNFYAELEEIYDEI |
| | YPVISLYNLVRNYVTQKPYSTKKIKLNFGIPTLADGWSKSKEYSNNAIILMRD |
| | NLYYLGIFNAKNKPDKKIIEGNTSENKGDYKKMIYNLLPGPNKMIPKVFLSSK |
| | TGVETYKPSAYILEGYKQNKHLKSSKDFDITFCHDLIDYFKNCIAIHPEWKNF |
| | GFDFSDTSTYEDISGFYREVELQGYKIDWTYISEKDIDLLQEKGQLYLFQIYNK |
| | DFSKKSSGNDNLHTMYLKNLFSEENLKDIVLKLNGEAEIFFRKSSIKNPIIHKK |
| | GSILVNRTYEAEEKDQFGNIQIVRKTIPENIYQELYKYFNDKSDKELSDEAAKL |
| | KNVVGHHEAATNIVKDYRYTYDKYFLHMPITINFKANKTSFINDRILQYIAKE |
| | KDLHVIGIDRGERNLIYVSVIDTCGNIVEQKSFNIVNGYDYQIKLKQQEGARQI |
| | ARKEWKEIGKIKEIKEGYLSLVIHEISKMVIKYNAIIAMEDLSYGFKKGRFKVE |
| | RQVYQKFETMLINKLNYLVFKDISITENGGLLKGYQLTYIPDKLKNVGHQCG |
| | CIFYVPAAYTSKIDPTTGFVNIFKFKDLTVDAKREFIKKFDSIRYDSDKNLFCFT |
| | FDYNNFITQNTVMSKSSWSVYTYGVRIKRRFVNGRFSNESDTIDITKDMEKTL |
| | EMTDINWRDGHDLRQDIIDYEIVQHIFEIFKLTVQMRNSLSELEDRDYDRLISP |
| | VLNENNIFYDSAKAGDALPKDADANGAYCIALKGLYEIKQITENWKEDGKFS |
| | RDKLKISNKDWFDFIQNKRYL (SEQ ID NO: 387) |
| CsCas12a- | MNYKTGLEDFIGKESLSKTLRNALIPTESTKIHMEEMGVIRDDELRAEKQQEL |
| previously | KEIMDDYYRAFIEEKLGQIQGIQWNSLFQKMEETMEDISVRKDLDKIQNEKR |
| known at Cpf1 | KEICCYFTSDKRFKDLFNAKLITDILPNFIKDNKEYTEEEKAEKEQTRVLFQRF |
| Clostridium sp. | ATAFTNYFNQRRNNFSEDNISTAISFRIVNENSEIHLQNMRAFQRIEQQYPEEV |
| AF34-10BH | CGMEEEYKDMLQEWQMKHIYLVDFYDRVLTQPGIEYYNGICGKINEHMNQF |
| Ref Seq. | CQKNRINKNDFRMKKLHKQILCKKSSYYEIPFRFESDQEVYDALNEFIKTMK |
| WP_118538418.1 | EKEIICRCVHLGQKCDDYDLGKIYISSNKYEQISNALYGSWDTIRKCIKEEYM |
| | DALPGKGEKKEEKAEAAAKKEEYRSIADIDKIISLYGSEMDRTISAKKCITEIC |
| | DMAGQISTDPLVCNSDIKLLQNKEKTTEIKTILDSFLHVYQWGQTFIVSDIIEK |
| | DSYFYSELEDVLEDFEGITTLYNHVRSYVTQKPYSTVKFKLHFGSPTLANGWS |
| | QSKEYDNNAILLMRDQKFYLGIFNVRNKPDKQIIKGHEKEEKGDYKKMIYNL |
| | LPGPSKMLPKVFITSRSGQETYKPSKHILDGYNEKRHIKSSPKFDLGYCWDLI |
| | DYYKECIHKHPDWKNYDFHFSDTKDYEDISGFYREVEMQGYQIKWTYISAD |
| | EIQKLDEKGQIFLFQIYNKDFSVHSTGKDNLHTMYLKNLFSEENLKDIVLKLN |
| | GEAELFFRKASIKTPVVHKKGSVLVNRSYTQTVGDKEIRVSIPEEYYTEIYNYL |
| | NHIGRGKLSTEAQRYLEERKIKSFTATKDIVKNYRYCCDHYFLHLPITINFKAK |
| | SDIAVNERTLAYIAKKEDIHIIGIDRGERNLLYISVVDVHGNIREQRSFNIVNGY |
| | DYQQKLKDREKSRDAARKNWEEIEKIKELKEGYLSMVIHYIAQLVVKYNAV |
| | VAMEDLNYGFKTGRFKVERQVYQKFETMLIEKLHYLVFKDREVCEEGGVLR |
| | GYQLTYIPESLKKVGKQCGFIFYVPAGYTSKIDPTTGFVNLFSFKNLTNRESRQ |
| | DFVGKFDEIRYDRDKKMFEFSFDYNNYIKKGTMLASTKWKVYTNGTRLKRI |
| | VVNGKYTSQSMEVELTDAMEKMLQRAGIEYHDGKDLKGQIVEKGIEAEIIDI |
| | FRLTVQMRNSRSESEDREYDRLISPVLNDKGEFFDTATADKTLPQDADANGA |
| | YCIALKGLYEVKQIKENWKENEQFPRNKLVQDNKTWFDFMQKKRYL (SEQ |
| | ID NO: 388) |
| BhCas 12b | MATRSFILKIEPNEEVKKGLWKTHEVLNHGIAYYMNILKLIRQEAIYEHHEQD |
| Bacillus | PKNPKKVSKAEIQAELWDFVLKMQKCNSFTHEVDKDEVFNILRELYEELVPSS |
| hisashii | VEKKGEANQLSNKFLYPLVDPNSQSGKGTASSGRKPRWYNLKIAGDPSWEEE |
| Ref Seq. | KKKWEEDKKKDPLAKILGKLAEYGLIPLFIPYTDSNEPIVKEIKWMEKSRNQS |
| WP_095142515.1 | VRRLDKDMFIQALERFLSWESWNLKVKEEYEKVEKEYKTLEERIKEDIQALK |
| | ALEQYEKERQEQLLRDTLNTNEYRLSKRGLRGWREIIQKWLKMDENEPSEK |
| | YLEVFKDYQRKHPREAGDYSVYEFLSKKENHFIWRNHPEYPYLYATFCEIDK |
| | KKKDAKQQATFTLADPINHPLWVRFEERSGSNLNKYRILTEQLHTEKLKKKLT |
| | VQLDRLIYPTESGGWEEKGKVDIVLLPSRQFYNQIFLDIEEKGKHAFTYKDESI |
| | KFPLKGTLGGARVQFDRDHLRRYPHKVESGNVGRIYFNMTVNIEPTESPVSK |
| | SLKIHRDDFPKVVNFKPKELTEWIKDSKGKKLKSGIESLEIGLRVMSIDLGQRQ |
| | AAAASIFEVVDQKPDIEGKLFFPIKGTELYAVHRASFNIKLPGETLVKSREVLR |
| | KAREDNLKLMNQKLNFLRNVLHFQQFEDITEREKRVTKWISRQENSDVPLVY |
| | QDELIQIRELMYKPYKDWVAFLKQLHKRLEVEIGKEVKHWRKSLSDGRKGL |
| | YGISLKNIDEIDRTRKFLLRWSLRPTEPGEVRRLEPGQRFAIDQLNHLNALKED |
| | RLKKMANTIIMHALGYCYDVRKKKWQAKNPACQIILFEDLSNYNPYEERSRF |
| | ENSKLMKWSRREIPRQVALQGEIYGLQVGEVGAQFSSRFHAKTGSPGIRCSV |
| | VTKEKLQDNRFFKNLQREGRLTLDKIAVLKEGDLYPDKGGEKFISLSKDRKC |
| | VTTHADINAAQNLQKRFWTRTHGFYKVYCKAYQVDGQTVYIPESKDQKQKI |
| | IEEFGEGYFILKDGVYEWVNAGKLKIKKGSSKQSSSELVDSDILKDSFDLASEL |
| | KGEKLMLYRDPSGNVFPSDKWMAAGVFFGKLERILISKLTNQYSISTIEDDSS |
| | KQSM (SEQ ID NO: 389) |
| ThCas12b | MSEKTTQRAYTLRLNRASGECAVCQNNSCDCWHDALWATHKAVNRGAKAF |
| Thermomonas | GDWLLTLRGGLCHTLVEMEVPAKGNNPPQRPTDQERRDRRVLLALSWLSVE |
| hydrothermalis | DEHGAPKEFIVATGRDSADDRAKKVEEKLREILEKRDFQEHEIDAWLQDCGPS |
| Ref Seq. | LKAHIREDAVWVNRRALFDAAVERIKTLTWEEAWDFLEPFFGTQYFAGIGDG |
| WP_072754838 | KDKDDAEGPARQGEKAKDLVQKAGQWLSARFGIGTGADFMSMAEAYEKIA |
| | KWASQAQNGDNGKATIEKLACALRPSEPPTLDTVLKCISGPGHKSATREYLKT |
| | LDKKSTVTQEDLNQLRKLADEDARNCRKKVGKKGKKPWADEVLKDVENSC |
| | ELTYLQDNSPARHREFSVMLDHAARRVSMAHSWIKKAEQRRRQFESDAQKL |
| | KNLQERAPSAVEWLDRFCESRSMTTGANTGSGYRIRKRAIEGWSYVVQAWA |
| | EASCDTEDKRIAAARKVQADPEIEKFGDIQLFEALAADEAICVWRDQEGTQN |
| | PSILIDYVTGKTAEHNQKRFKVPAYRHPDELRHPVFCDFGNSRWSIQFAIHKEI |
| | RDRDKGAKQDTRQLQNRHGLKMRLWNGRSMTDVNLHWSSKRLTADLALD |
| | QNPNPNPTEVTRADRLGRAASSAFDHVKIKNVFNEKEWNGRLQAPRAELDRI |
| | AKLEEQGKTEQAEKLRKRLRWYVSFSPCLSPSGPFIVYAGQHNIQPKRSGQYA |
| | PHAQANKGRARLAQLILSRLPDLRILSVDLGHRFAAACAVWETLSSDAFRREI |
| | QGLNVLAGGSGEGDLFLHVEMTGDDGKRRTVVYRRIGPDQLLDNTPHPAPW |
| | ARLDRQFLIKLQGEDEGVREASNEELWTVHKLEVEVGRTVPLIDRMVRSGFG |
| | KTEKQKERLKKLRELGWISAMPNEPSAETDEKEGEIRSISRSVDELMSSALGT |
| | LRLALKRHGNRARIAFAMTADYKPMPGGQKYYFHEAKEASKNDDETKRRD |
| | NQIEFLQDALSLWHDLFSSPDWEDNEAKKLWQNHIATLPNYQTPEEISAELKR |
| | VERNKKRKENRDKLRTAAKALAENDQLRQHLHDTWKERWESDDQQWKER |
| | LRSLKDWIFPRGKAEDNPSIRHVGGLSITRINTISGLYQILKAFKMRPEPDDLR |
| | KNIPQKGDDELENFNRRLLEARDRLREQRVKQLASRIIEAALGVGRIKIPKNG |
| | KLPKRPRTTVDTPCHAVVIESLKTYRPDDLRTRRENRQLMQWSSAKVRKYLK |
| | EGCELYGLHFLEVPANYTSRQCSRTGLPGIRCDDVPTGDFLKAPWWRRAINT |
| | AREKNGGDAKDRFLVDLYDHLNNLQSKGEALPATVRVPRQGGNLFIAGAQL |
| | DDTNKERRAIQADLNAAANIGLRALLDPDWRGRWWYVPCKDGTSEPALDRI |
| | EGSTAFNDVRSLPTGDNSSRRAPREIENLWRDPSGDSLESGTWSPTRAYWDT |
| | VQSRVIELLRRHAGLPTS (SEQ ID NO: 390) |
| LsCas12b | MSIRSFKLKLKTKSGVNAEQLRRGLWRTHQLINDGIAYYMNWLVLLRQEDLF |
| Laceyella | IRNKETNEIEKRSKEEIQAVLLERVHKQQQRNQWSGEVDEQTLLQALRQLYEE |
| sacchari | IVPSVIGKSGNASLKARFFLGPLVDPNNKTTKDVSKSGPTPKWKKMKDAGDP |
| WP_132221894.1 | NWVQEYEKYMAERQTLVRLEEMGLIPLFPMYTDEVGDIHWLPQASGYTRTW |
| | DRDMFQQAIERLLSWESWNRRVRERRAQFEKKTHDFASRFSESDVQWMNKL |
| | REYEAQQEKSLEENAFAPNEPYALTKKALRGWERVYHSWMRLDSAASEEAY |
| | WQEVATCQTAMRGEFGDPAIYQFLAQKENHDIWRGYPERVIDFAELNHLQRE |
| | LRRAKEDATFTLPDSVDHPLWVRYEAPGGTNIHGYDLVQDTKRNLTLILDKFI |
| | LPDENGSWHEVKKVPFSLAKSKQFHRQVWLQEEQKQKKREVVFYDYSTNLP |
| | HLGTLAGAKLQWDRNFLNKRTQQQIEETGEIGKVFFNISVDVRPAVEVKNGR |
| | LQNGLGKALTVLTHPDGTKIVTGWKAEQLEKWVGESGRVSSLGLDSLSEGLR |
| | VMSIDLGQRTSATVSVFEITKEAPDNPYKFFYQLEGTEMFAVHQRSFLLALPG |
| | ENPPQKIKQMREIRWKERNRIKQQVDQLSAILRLHKKVNEDERIQAIDKLLQK |
| | VASWQLNEEIATAWNQALSQLYSKAKENDLQWNQAIKNAHHQLEPVVGKQI |
| | SLWRKDLSTGRQGIAGLSLWSIEELEATKKLLTRWSKRSREPGVVKRIERFETF |
| | AKQIQHHINQVKENRLKQLANLIVMTALGYKYDQEQKKWIEVYPACQVVLF |
| | ENLRSYRFSFERSRRENKKLMEWSHRSIPKLVQMQGELFGLQVADVYAAYSS |
| | RYHGRTGAPGIRCHALTEADLRNETNIIHELIEAGFIKEEHRPYLQQGDLVPWS |
| | GGELFATLQKPYDNPRILTLHADINAAQNIQKRFWHPSMWFRVNCESVMEGE |
| | IVTYVPKNKTVHKKQGKTFRFVKVEGSDVYEWAKWSKNRNKNTFSSITERK |
| | PPSSMILFRDPSGTFFKEQEWVEQKTFWGKVQSMIQAYMKKTIVQRMEE |
| | (SEQ ID NO: 391) |
| DtCas12b | MVLGRKDDTAELRRALWTTHEHVNLAVAEVERVLLRCRGRSYWTLDRRGDP |
| Dsulfonatronum | VHVPESQVAEDALAMAREAQRRNGWPVVGEDEEILLALRYLYEQIVPSCLLD |
| thiodismutans | DLGKPLKGDAQKIGTNYAGPLFDSDTCRRDEGKDVACCGPFHEVAGKYLGA |
| WP_031386437 | LPEWATPISKQEFDGKDASHLRFKATGGDDAFFRVSIEKANAWYEDPANQDA |
| | LKNKAYNKDDWKKEKDKGISSWAVKYIQKQLQLGQDPRTEVRRKLWLELGL |
| | LPLFIPVFDKTMVGNLWNRLAVRLALAHLLSWESWNHRAVQDQALARAKR |
| | DELAALFLGMEDGFAGLREYELRRNESIKQHAFEPVDRPYVVSGRALRSWTR |
| | VREEWLRHGDTQESRKNICNRLQDRLRGKFGDPDVFHWLAEDGQEALWKE |
| | RDCVTSFSLLNDADGLLEKRKGYALMTFADARLHPRWAMYEAPGGSNLRTY |
| | QIRKTENGLWADVVLLSPRNESAAVEEKTFNVRLAPSGQLSNVSFDQIQKGSK |
| | MVGRCRYQSANQQFEGLLGGAEILFDRKRIANEQHGATDLASKPGHVWFKL |
| | TLDVRPQAPQGWLDGKGRPALPPEAKHFKTALSNKSKFADQVRPGLRVLSVD |
| | LGVRSFAACSVFELVRGGPDQGTYFPAADGRTVDDPEKLWAKHERSFKITLPG |
| | ENPSRKEEIARRAAMEELRSLNGDIRRLKAILRLSVLQEDDPRTEHLRLFMEAI |
| | VDDPAKSALNAELFKGFGDDRFRSTPDLWKQHCHFFHDKAEKVVAERFSRW |
| | RTETRPKSSSWQDWRERRGYAGGKSYWAVTYLEAVRGLILRWNMRGRTYGE |
| | VNRQDKKQFGTVASALLHHINQLKEDRIKTGADMIIQAARGFVPRKNGAGW |
| | VQVHEPCRLILFEDLARYRFRTDRSRRENSRLMRWSHREIVNEVGMQGELYG |
| | LHVDTTEAGFSSRYLASSGAPGVRCRHLVEEDFHDGLPGMHLVGELDWLLP |
| | KDKDRTANEARRLLGGMVRPGMLVPWDGGELFATLNAASQLHVIHADINAA |
| | QNLQRRFWGRCGEAIRIVCNQLSVDGSTRYEMAKAPKARLLGALQQLKNGD |
| | APFHLTSIPNSQKPENSYVMTPTNAGKKYRAGPGEKSSGEEDELALDIVEQAE |
| | ELAQGRKTFFRDPSGVFFAPDRWLPSEIYWSRIRRRIWQVTLERNSSGRQERA |
| | EMDEMPY (SEQ ID NO: 392) |

The base editors described herein may also comprise Cas12a/Cpf1 (dCpf1) variants that may be used as a guide nucleotide sequence-programmable DNA-binding protein domain. The Cas12a/Cpf1 protein has a RuvC-like endonuclease domain that is similar to the RuvC domain of Cas9 but does not have a HNH endonuclease domain, and the N-terminal of Cpf1 does not have the alpha-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity.

Recently, a more specific SpCas9 variant termed Sniper-Cas9 was generated, in Lee, J. K. et al., Nat. Commun. 9, 3048 (2018), which is incorporated by reference herein. Sniper-Cas9 was shown to significantly lower off-target editing than with SpCas9 and wild-type-like levels of on-target activities with truncated sgRNAs or sgRNAs with 5′-G-extended mismatched spacers. The Sniper-SpCas9 contains D10A, F539S, M763I, and K890N substitutions in the amino acid sequence of SEQ ID NO: 6 (and is thus also a nickase, and is thus referred to herein also as “Sniper-nCas9”). Accordingly, in some embodiments, the napDNAbp domain of any of the disclosed CGBEs is a Sniper-nCas9, such as a Sniper-SpCas9n (SEQ ID NO: 733).

Recently, Cas9 variants SpG and SpRY that were generated from the SpCas9 sequence that can target almost all PAMs, exhibiting robust activities on a wide range of sites with NRN PAMs in human cells and lower but substantial activity on those with NYN PAMs, in Walton et al., Science. 2020; 368(6488): 290-296, which is incorporated by reference herein. The SpG Cas9 variant contains D1135L, S1136W, G1218K, E1219Q, R1335Q, and T1337R substitutions in the amino acid sequence of SEQ ID NO: 6. The SpRY Cas9 variant contains L1111R, D1135L, S1136W, G1218K, E1219Q, N1317R, A1322R, R1333P, R1335Q, and T1337R substitutions in the amino acid sequence of SEQ ID NO: 6. Accordingly, in some embodiments, the napDNAbp domain of any of the disclosed CGBEs is an SpG or an SpRY Cas9 variant, or a variant thereof.

The disclosure also provides fragments of napDNAbps, such as truncations of any of the napDNAbps provided herein. In some embodiments, the napDNAbp is an N-terminal truncation, where one or more amino acids are absent from the N-terminus of the napDNAbp. In some embodiments, the napDNAbp is absent 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the N-terminus of the napDNAbp. For example, the N-terminal truncation of the napDNAbp may be an N-terminal truncation of any napDNAbp provided herein, such as any one of the napDNAbps provided in any one of SEQ ID NOs: 4-40, 726-736. In some embodiments, the napDNAbp is a C-terminal truncation, where one or more amino acids are absent from the C-terminus of the napDNAbp. In some embodiments, the napDNAbp is absent 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the C-terminus of the napDNAbp. For example, the C-terminal truncation of the napDNAbp may be a C-terminal truncation of any napDNAbp provided herein, such as any one of the NAPs provided in any one of SEQ ID NOs: 4-40, 726-736.

In some embodiments, any of the napDNAbps provided herein have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to any napDNAbp provided herein, such as any one of the napDNAbps provided in SEQ ID NOs: 4-40, 726-736.

### Uracil Binding Proteins (UBP)

The disclosed CGBEs contain at least one uracil binding protein (UBP) domain(s). The disclosed CGBEs may comprise two or more UBP domains. In some embodiments, the disclosed CGBEs comprise two UBP domains, such as two UdgX protein domains. In particular embodiments, the disclosed CGBEs comprise one or two UBP domains each comprising the amino acid sequence of SEQ ID NO: 49. In some embodiments, the disclosed CGBEs comprise one or two UBP domains each comprising a variant of the UdgX protein.

A uracil binding protein, or UBP, refers to a protein that is capable of binding to uracil. In some embodiments, the uracil binding protein is a uracil modifying enzyme. In some embodiments, the uracil binding protein is a uracil base excision enzyme. In some embodiments, the uracil binding protein is a uracil DNA glycosylase (UDG). In some embodiments, a uracil binding protein binds uracil with an affinity that is at least 1%, 2%, 3%, 5%, 10%, 15%, 20%, 30%, 40%, 50%, 60%, 70%, 80%, 90%, or at least 95% of the affinity that a wild type UDG (e.g., a human UDG) binds to uracil. In some embodiments, the uracil binding protein may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type uracil binding protein such as a wild type UDG (e.g., a human UDG) binds to uracil.

In some embodiments, the UBP is a uracil modifying enzyme. In some embodiments, the UBP is a uracil base excision enzyme. In some embodiments, the UBP is a uracil DNA glycosylase. In some embodiments, the UBP is any of the uracil binding proteins provided herein. For example, the UBP may be a UDG, a UdgX, a UdgX*, a UdgX_On, or a SMUG1. In some embodiments, the UBP comprises an amino acid sequence that is at least 75%, 80%, 85%, 90%, 95%, 96%, 97%, 98%, 99%, or 99.5% identical to a uracil binding protein, a uracil base excision enzyme or a uracil DNA glycosylase (UDG) enzyme. In some embodiments, the UBP comprises an amino acid sequence that is at least 75%, 80%, 85%, 90%, 95%, 96%, 97%, 98%, 99%, or 99.5% identical to any of the uracil binding proteins provided herein, for example, any of the UBP and UBP variants provided below. In some embodiments, the UBP comprises an amino acid sequence that is at least 75%, 80%, 85%, 90%, 95%, 96%, 97%, 98%, 99%, or 99.5% identical to any one of SEQ ID NOs: 48-53. In some embodiments, the UBP comprises the amino acid sequence of any one of SEQ ID NOs: 48-53. In some embodiments, the uracil binding protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to any UBP provided herein, such as any one of SEQ ID NOs: 48-53.

The disclosed CGBEs may comprise one or two (or more) UBP domains each comprising an amino acid sequence that is at least 75%, 80%, 85%, 90%, 95%, 96%, 97%, 98%, 99%, or 99.5% identical to the sequence of SEQ ID NO: 49. In some embodiments, the disclosed CGBEs comprise one or two UBP domains each comprising the amino acid sequence of SEQ ID NO: 49.

The disclosure also provides fragments of UBPs, such as truncations of any of the UBPs provided herein. In some embodiments, the UBP is an N-terminal truncation, where one or more amino acids are absent from the N-terminus of the UBP. In some embodiments, the UBP is absent 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the N-terminus of the UBP. For example, the N-terminal truncation of the UBP may be an N-terminal truncation of any UBP provided herein, such as any one of the UBPs provided in any one of SEQ ID NOs: 48-53. In some embodiments, the UBP is a C-terminal truncation, where one or more amino acids are absent from the C-terminus of the UBP. In some embodiments, the UBP is absent 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the C-terminus of the UBP. For example, the C-terminal truncation of the UBP may be a C-terminal truncation of any UBP provided herein, such as any one of the UBPs provided in any one of SEQ ID NOs: 48-53.

It should be appreciated that other UBPs would be apparent to the skilled artisan and are within the scope of this disclosure. For example UBPs have been described previously in Sang et al., “A Unique Uracil-DNA binding protein of the uracil DNA glycosylase superfamily,” Nucleic Acids Research, Vol. 43, No. 17 2015; the entire contents of which are hereby incorporated by reference.

UDG
(SEQ ID NO: 48)
MIGQKTLYSFFSPSPARKRHAPSPEPAVQGTGVAGVPEESGDAAAIPAK
KAPAGQEEPGTPPSSPLSAEQLDRIQRNKAAALLRLAARNVPVGFGESW
KKHLSGEFGKPYFIKLMGFVAEERKHYTVYPPPHQVFTWTQMCDIKDVK
VVILGQDPYHGPNQAHGLCFSVQRPVPPPPSLENIYKELSTDIEDFVHP
GHGDLSGWAKQGVLLLNAVLTVRAHQANSHKERGWEQFTDAVVSWLNQN
SNGLVFLLWGSYAQKKGSAIDRKRHHVLQTAHPSPLSVYRGFFGCRHFS
KTNELLQKSGKKPIDWKEL
UdgX
(SEQ ID NO: 49)
MAGAQDFVPHTADLAELAAAAGECRGCGLYRDATQAVFGAGGRSARIMM
IGEQPGDKEDLAGLPFVGPAGRLLDRALEAADIDRDALYVTNAVKHFKF
TRAAGGKRRIHKTPSRTEVVACRPWLIAEMTSVEPDVVVLLGATAAKAL
LGNDFRVTQHRGEVLHVDDVPGDPALVATVHPSSLLRGPKEERESAFAG
LVDDLRVAADVRP
UdgX* (R107S)
(SEQ ID NO: 50)
MAGAQDFVPHTADLAELAAAAGECRGCGLYRDATQAVFGAGGRSARIMM
IGEQPGDKEDLAGLPFVGPAGRLLDRALEAADIDRDALYVTNAVKHFKF
TRAAGGKRSIHKTPSRTEVVACRPWLIAEMTSVEPDVVVLLGATAAKAL
LGNDFRVTQHRGEVLHVDDVPGDPALVATVHPSSLLRGPKEERESAFAG
LVDDLRVAADVRP
UdgX_On (H109S)
(SEQ ID NO: 51)
MAGAQDFVPHTADLAELAAAAGECRGCGLYRDATQAVFGAGGRSARIMM
IGEQPGDKEDLAGLPFVGPAGRLLDRALEAADIDRDALYVTNAVKHFKF
TRAAGGKRRISKTPSRTEVVACRPWLIAEMTSVEPDVVVLLGATAAKAL
LGNDFRVTQHRGEVLHVDDVPGDPALVATVHPSSLLRGPKEERESAFAG
LVDDLRVAADVRP
Rev7
(SEQ ID NO: 52)
MTTLTRQDLNFGQVVADVLCEFLEVAVHLILYVREVYPVGIFQKRKKYN
VPVQMSCHPELNQYIQDTLHCVKPLLEKNDVEKVVVVILDKEHRPVEKF
VFEITQPPLLSISSDSLLSHVEQLLRAFILKISVCDAVLDHNPPGCTFT
VLVHTREAATRNMEKIQVIKDFPWILADEQDVHMHDPRLIPLKTMTSDI
LKMQLYVEERAHKGS
Smug1
(SEQ ID NO: 53)
MPQAFLLGSIHEPAGALMEPQPCPGSLAESFLEEELRLNAELSQLQFSE
PVGIIYNPVEYAWEPHRNYVTRYCQGPKEVLFLGMNPGPFGMAQTGVPF
GEVSMVRDWLGIVGPVLTPPQEHPKRPVLGLECPQSEVSGARFWGFFRN
LCGQPEVFFHHCFVHNLCPLLFLAPSGRNLTPAELPAKQREQLLGICDA
ALCRQVQLLGVRLVVGVGRLAEQRARRALAGLMPEVQVEGLLHPSPRNP
QANKGWEAVAKERLNELGLLPLLLK

### DNA Repair Protein Domains

As used herein, a DNA repair protein refers to an enzyme or protein that is implicated in DNA repair. The DNA repair protein domains of this disclosure were identified following a CRISPR interference screen of mammalian genes implicated in DNA repair that further impact cytosine base editing efficiency and purity. It will be appreciated that DNA repair proteins other than those enumerated herein may be incorporated into the disclosed CGBEs. It will be appreciated that the DNA repair proteins for use in any of the disclosed CGBEs may be other protein components of DNA repair pathways and/or DNA repair enzymes or cofactors. The CRISPRi screen provided in Example 7 of this disclosure may provide additional hits for DNA repair proteins useful in any of the disclosed base editors and methods for editing. Other protein screens known to those in the art may provide additional hits for DNA repair proteins useful in any of the disclosed base editors and methods for editing.

In some embodiments, the DNA repair protein domain is a mammalian (such as a human) DNA repair protein. In some embodiments, the DNA repair protein domain is a human DNA polymerase, such as a human translesion polymerase. In some embodiments, the DNA repair protein is a human exonuclease. In some embodiments, the DNA repair protein is a human E3 ligase. The DNA repair protein may be selected from a DNA polymerase, an exonuclease, an RNA binding motif protein, an E3 ligase, and a translesion polymerase. In particular embodiments, the DNA repair protein is one of POLD2, RBMX, and EXO1.

In some embodiments, the DNA repair protein is a nucleic acid polymerase, such as a DNA polymerase (e.g., a translesion polymerase). In various embodiments, the DNA repair protein is selected from DNA polymerase D1 (POLD1), DNA polymerase D2 (POLD2), and DNA polymerase D3 (POLD3). In some embodiments, the DNA repair protein is an RNA binding motif protein, such as RNA binding motif protein, X-linked (RBMX). In some embodiments, the DNA repair protein is an exonuclease, such as exonuclease 1 (EX01). In some embodiments, the DNA repair protein is an E3 ligase, such as RAD18 or RFWD3.

In some embodiments, the DNA repair protein is a protein encoded by a gene selected from DDX1, EXO1, POLD1, POLD2, POLD3, RAD18, RBMX, REV1, RFWD3, TIMELESS, PCNA, POLH, POLK, UBE2I, and UBE2T.

In some embodiments, the DNA repair protein domain comprises an amino acid sequence that is at least 75%, 80%, 85%, 90%, 95%, 96%, 97%, 98%, 99%, or 99.5% identical to any one of SEQ ID NOs: 708-723. In some embodiments, the DNA repair protein domain comprises the amino acid sequence of any one of SEQ ID NOs: 708-723. In some embodiments, the DNA repair protein domain has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to any NAP provided herein, such as any one of SEQ ID NOs: 708-723.

In particular embodiments, the DNA repair protein is one of POLD2, RBMX, and EXO1. In some embodiments, the DNA repair protein domain comprises an amino acid sequence that is at least 75%, 80%, 85%, 90%, 95%, 96%, 97%, 98%, 99%, or 99.5% identical to any one of SEQ ID NOs: 709, 712, and 717. In some embodiments, the DNA repair protein domain comprises the amino acid sequence of any one of SEQ ID NOs: 709, 712, and 717.

### Nucleic Acid Polymerases (NAP)

A nucleic acid polymerase, or NAP, refers to an enzyme that synthesizes nucleic acid molecules (e.g., DNA and RNA) from nucleotides (e.g., deoxyribonucleotides and ribonucleotides). In some embodiments, the NAP is a DNA polymerase. In some embodiments, the NAP is a translesion polymerase. Translesion polymerases play a role in mutagenesis, for example, by restarting replication forks or filling in gaps that remain in the genome due to the presence of DNA lesions. Exemplary translesion polymerases include, without limitation, Pol Beta, Pol Lambda, Pol Eta, Pol Mu, Pol Iota, Pol Kappa, Pol Alpha, Pol Delta, Pol Gamma, and Pol Nu.

In some embodiments, the NAP is a eukaryotic nucleic acid polymerase. In some embodiments, the NAP is a DNA polymerase. In some embodiments, the NAP has translesion polymerase activity. In some embodiments, the NAP is a translesion DNA polymerase. In some embodiments, the NAP is a Rev7, Rev1 complex, polymerase iota, polymerase kappa, or polymerase eta. In some embodiments, the NAP is a eukaryotic polymerase alpha, beta, gamma, delta, epsilon, gamma, eta, iota, kappa, lambda, mu, or nu. In some embodiments, the NAP comprises an amino acid sequence that is at least 75%, 80%, 85%, 90%, 95%, 96%, 97%, 98%, 99%, or 99.5% identical to a naturally occurring nucleic acid polymerase (e.g., a translesion DNA polymerase). In some embodiments, the NAP comprises an amino acid sequence that is at least 75%, 80%, 85%, 90%, 95%, 96%, 97%, 98%, 99%, or 99.5% identical to any of the nucleic acid polymerases provided herein, e.g., below. For example, the NAP may comprise an amino acid sequence that is at least 75%, 80%, 85%, 90%, 95%, 96%, 97%, 98%, 99%, or 99.5% identical to any one of SEQ ID NOs: 54-64. In some embodiments, the NAP comprises the amino acid sequence of any one of SEQ ID NOs: 54-64. It should be appreciated that other NAPs would be apparent to the skilled artisan and are within the scope of this disclosure. In some embodiments, the NAP comprises the amino acid sequence of any one of SEQ ID NOs: 54-64. In some embodiments, the nucleic acid polymerase has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to any NAP provided herein, such as any one of SEQ ID NOs: 54-64.

It should be appreciated that other translesion polymerases that preferentially integrate non-C nucleobases (e.g., adenine, guanine, and thymine), may be used to generate alternative mutations (e.g., C to A mutations). Accordingly, in some embodiments, bases other than cytosine (e.g., adenine, guanine, or thymine) may replace a nucleobase opposite an abasic site.

The disclosure also provides fragments of NAPs, such as truncations of any of the NAPs provided herein. In some embodiments, the NAP is an N-terminal truncation, where one or more amino acids are absent from the N-terminus of the NAP. In some embodiments, the NAP is absent 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the N-terminus of the NAP. For example, the N-terminal truncation of the NAP may be an N-terminal truncation of any NAP provided herein, such as any one of the NAPs provided in any one of SEQ ID NOs: 54-64. In some embodiments, the NAP is a C-terminal truncation, where one or more amino acids are absent from the C-terminus of the NAP. In some embodiments, the NAP is absent 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the C-terminus of the NAP. For example, the C-terminal truncation of the NAP may be a C-terminal truncation of any NAP provided herein, such as any one of the NAPs provided in any one of SEQ ID NOs: 54-64.

Pol Beta
(SEQ ID NO: 54)
MSKRKAPQETLNGGITDMLTELANFEKNVSQAIHKYNAYRKAASVIAKYPHKIKSGAEAKK
LPGVGTKIAEKIDEFLATGKLRKLEKIRQDDTSSSINFLTRVSGIGPSAARKFVDEGIKTLEDLR
KNEDKLNHHQRIGLKYFGDFEKRIPREEMLQMQDIVLNEVKKVDSEYIATVCGSFRRGAESS
GDMDVLLTHPSFTSESTKQPKLLHQVVEQLQKVHFITDTLSKGETKFMGVCQLPSKNDEKEY
PHRRIDIRLIPKDQYYCGVLYFTGSDIFNKNMRAHALEKGFTINEYTIRPLGVTGVAGEPLPVD
SEKDIFDYIQWKYREPKDRSE
Pol Lambda
(SEQ ID NO: 55)
MDPRGILKAFPKRQKIHADASSKVLAKIPRREEGEEAEEWLSSLRAHVVRTGIGRARAELFEK
QIVQHGGQLCPAQGPGVTHIVVDEGMDYERALRLLRLPQLPPGAQLVKSAWLSLCLQERRL
VDVAGFSIFIPSRYLDHPQPSKAEQDASIPPGTHEALLQTALSPPPPPTRPVSPPQKAKEAPNTQ
AQPISDDEASDGEETQVSAADLEALISGHYPTSLEGDCEPSPAPAVLDKWVCAQPSSQKATN
HNLHITEKLEVLAKAYSVQGDKWRALGYAKAINALKSFHKPVTSYQEACSIPGIGKRMAEKII
EILESGHLRKLDHISESVPVLELFSNIWGAGTKTAQMWYQQGFRSLEDIRSQASLTTQQAIGL
KHYSDFLERMPREEATEIEQTVQKAAQAFNSGLLCVACGSYRRGKATCGDVDVLITHPDGRS
HRGIFSRLLDSLRQEGFLTDDLVSQEENGQQQKYLGVCRLPGPGRRHRRLDIIVVPYSEFACA
LLYFTGSAHFNRSMRALAKTKGMSLSEHALSTAVVRNTHGCKVGPGRVLPTPTEKDVFRLL
GLPYREPAERDW
Pol Eta
(SEQ ID NO: 56)
MATGQDRVVALVDMDCFFVQVEQRQNPHLRNKPCAVVQYKSWKGGGIIAVSYEARAFGVT
RSMWADDAKKLCPDLLLAQVRESRGKANLTKYREASVEVMEIMSRFAVIERASIDEAYVDL
TSAVQERLQKLQGQPISADLLPSTYIEGLPQGPTTAEETVQKEGMRKQGLFQWLDSLQIDNLT
SPDLQLTVGAVIVEEMRAAIERETGFQCSAGISHNKVLAKLACGLNKPNRQTLVSHGSVPQLF
SQMPIRKIRSLGGKLGASVIEILGIEYMGELTQFTESQLQSHFGEKNGSWLYAMCRGIEHDPV
KPRQLPKTIGCSKNFPGKTALATREQVQWWLLQLAQELEERLTKDRNDNDRVATQLVVSIR
VQGDKRLSSLRRCCALTRYDAHKMSHDAFTVIKNCNTSGIQTEWSPPLTMLFLCATKFSASA
PSSSTDITSFLSSDPSSLPKVPVTSSEAKTQGSGPAVTATKKATTSLESFFQKAAERQKVKEAS
LSSLTAPTQAPMSNSPSKPSLPFQTSQSTGTEPFFKQKSLLLKQKQLNNSSVSSPQQNPWSNCK
ALPNSLPTEYPGCVPVCEGVSKLEESSKATPAEMDLAHNSQSMHASSASKSVLEVTQKATPN
PSLLAAEDQVPCEKCGSLVPVWDMPEHMDYHFALELQKSFLQPHSSNPQVVSAVSHQGKRN
PKSPLACTNKRPRPEGMQTLESFFKPLTH
Pol Mu
(SEQ ID NO: 57)
MLPKRRRARVGSPSGDAASSTPPSTRFPGVAIYLVEPRMGRSRRAFLTGLARSKGFRVLDACS
SEATHVVMEETSAEEAVSWQERRMAAAPPGCTPPALLDISWLTESLGAGQPVPVECRHRLEV
AGPRKGPLSPAWMPAYACQRPTPLTHHNTGLSEALEILAEAAGFEGSEGRLLTFCRAASVLK
ALPSPVTTLSQLQGLPHFGEHSSRVVQELLEHGVCEEVERVRRSERYQTMKLFTQIFGVGVKT
ADRWYREGLRTLDDLREQPQKLTQQQKAGLQHHQDLSTPVLRSDVDALQQVVEEAVGQAL
PGATVTLTGGFRRGKLQGHDVDFLITHPKEGQEAGLLPRVMCRLQDQGLILYHQHQHSCCES
PTRLAQQSHMDAFERSFCIFRLPQPPGAAVGGSTRPCPSWKAVRVDLVVAPVSQFPFALLGW
TGSKLFQRELRRFSRKEKGLWLNSHGLFDPEQKTFFQAASEEDIFRHLGLEYLPPEQRNA
Pol Iota
(SEQ ID NO: 58)
MEKLGVEPEEEGGGDDDEEDAEAWAMELADVGAAASSQGVHDQVLPTPNASSRVIVHVDL
DCFYAQVEMISNPELKDKPLGVQQKYLVVTCNYEARKLGVKKLMNVRDAKEKCPQLVLVN
GEDLTRYREMSYKVTELLEEFSPVVERLGFDENFVDLTEMVEKRLQQLQSDELSAVTVSGHV
YNNQSINLLDVLHIRLLVGSQIAAEMREAMYNQLGLTGCAGVASNKLLAKLVSGVFKPNQQ
TVLLPESCQHLIHSLNHIKEIPGIGYKTAKCLEALGINSVRDLQTFSPKILEKELGISVAQRIQKL
SFGEDNSPVILSGPPQSFSEEDSFKKCSSEVEAKNKIEELLASLLNRVCQDGRKPHTVRLIIRRY
SSEKHYGRESRQCPIPSHVIQKLGTGNYDVMTPMVDILMKLFRNMVNVKMPFHLTLLSVCFC
NLKALNTAKKGLIDYYLMPSLSTTSRSGKHSFKMKDTHMEDFPKDKETNRDFLPSGRIESTR
TRESPLDTTNFSKEKDINEFPLCSLPEGVDQEVFKQLPVDIQEEILSGKSREKFQGKGSVSCPLH
ASRGVLSFFSKKQMQDIPINPRDHLSSSKQVSSVSPCEPGTSGFNSSSSSYMSSQKDYSYYLDN
RLKDERISQGPKEPQGFHFTNSNPAVSAFHSFPNLQSEQLFSRNHTTDSHKQTVATDSHEGLT
ENREPDSVDEKITFPSDIDPQVFYELPEAVQKELLAEWKRAGSDFHIGHK
Pol Kappa
(SEQ ID NO: 59)
MDSTKEKCDSYKDDLLLRMGLNDNKAGMEGLDKEKINKIIMEATKGSRFYGNELK
KEKQVNQRIENMMQQKAQITSQQLRKAQLQVDRFAMELEQSRNLSNTIVHIDMDAF
YAAVEMRDNPELKDKPIAVGSMSMLSTSNYHARRFGVRAAMPGFIAKRLCPQLIIVP
PNFDKYRAVSKEVKEILADYDPNFMAMSLDEAYLNITKHLEERQNWPEDKRRYFIK
MGSSVENDNPGKEVNKLSEHERSISPLLFEESPSDVQPPGDPFQVNFEEQNNPQILQN
SVVFGTSAQEVVKEIRFRIEQKTTLTASAGIAPNTMLAKVCSDKNKPNGQYQILPNRQ
AVMDFIKDLPIRKVSGIGKVTEKMLKALGIITCTELYQQRALLSLLFSETSWHYFLHIS
LGLGSTHLTRDGERKSMSVERTFSEINKAEEQYSLCQELCSELAQDLQKERLKGRTV
TIKLKNVNFEVKTRASTVSSVVSTAEEIFAIAKELLKTEIDADFPHPLRLRLMGVRISSF
PNEEDRKHQQRSIIGFLQAGNQALSATECTLEKTDKDKFVKPLEMSHKKSFFDKKRS
ERKWSHQDTFKCEAVNKQSFQTSQPFQVLKKKMNENLEISENSDDCQILTCPVCFRA
QGCISLEALNKHVDECLDGPSISENFKMFSCSHVSATKVNKKENVPASSLCEKQDYE
AHPKIKEISSVDCIALVDTIDNSSKAESIDALSNKHSKEECSSLPSKSFNIEHCHQNSSS
TVSLENEDVGSFRQEYRQPYLCEVKTGQALVCPVCNVEQKTSDLTLFNVHVDVCLN
KSFIQELRKDKFNPVNQPKESSRSTGSSSGVQKAVTRTKRPGLMTKYSTSKKIKPNNP
KHTLDIFFK
Pol Alpha
(SEQ ID NO: 60)
MAPVHGDDCEIGASALSDSGSFVSSRARREKKSKKGRQEALERLKKAKAGEKYKYEVEDFT
GVYEEVDEEQYSKLVQARQDDDWIVDDDGIGYVEDGREIFDDDLEDDALDADEKGKDGKA
RNKDKRNVKKLAVTKPNNIKSMFIACAGKKTADKAVDLSKDGLLGDILQDLNTETPQITPPP
VMILKKKRSIGASPNPFSVHTATAVPSGKIASPVSRKEPPLTPVPLKRAEFAGDDVQVESTEEE
QESGAMEFEDGDFDEPMEVEEVDLEPMAAKAWDKESEPAEEVKQEADSGKGTVSYLGSFLP
DVSCWDIDQEGDSSFSVQEVQVDSSHLPLVKGADEEQVFHFYWLDAYEDQYNQPGVVFLFG
KVWIESAETHVSCCVMVKNIERTLYFLPREMKIDLNTGKETGTPISMKDVYEEFDEKIATKYK
IMKFKSKPVEKNYAFEIPDVPEKSEYLEVKYSAEMPQLPQDLKGETFSHVFGTNTSSLELFLM
NRKIKGPCWLEVKSPQLLNQPVSWCKVEAMALKPDLVNVIKDVSPPPLVVMAFSMKTMQN
AKNHQNEIIAMAALVHHSFALDKAAPKPPFQSHFCVVSKPKDCIFPYAFKEVIEKKNVKVEV
AATERTLLGFFLAKVHKIDPDIIVGHNIYGFELEVLLQRINVCKAPHWSKIGRLKRSNMPKLG
GRSGFGERNATCGRMICDVEISAKELIRCKSYHLSELVQQILKTERVVIPMENIQNMYSESSQL
LYLLEHTWKDAKFILQIMCELNVLPLALQITNIAGNIMSRTLMGGRSERNEFLLLHAFYENNY
IVPDKQIFRKPQQKLGDEDEEIDGDTNKYKKGRKKAAYAGGLVLDPKVGFYDKFILLLDENS
LYPSIIQEFNICFTTVQRVASEAQKVTEDGEQEQIPELPDPSLEMGILPREIRKLVERRKQVKQL
MKQQDLNPDLILQYDIRQKALKLTANSMYGCLGFSYSRFYAKPLAALVTYKGREILMHTKE
MVQKMNLEVIYGDTDSIMINTNSTNLEEVFKLGNKVKSEVNKLYKLLEIDIDGVFKSLLLLK
KKKYAALVVEPTSDGNYVTKQELKGLDIVRRDWCDLAKDTGNFVIGQILSDQSRDTIVENIQ
KRLIEIGENVLNGSVPVSQFEINKALTKDPQDYPDKKSLPHVHVALWINSQGGRKVKAGDTV
SYVICQDGSNLTASQRAYAPEQLQKQDNLTIDTQYYLAQQIHPVVARICEPIDGIDAVLIATW
LGLDPTQFRVHHYHKDEENDALLGGPAQLTDEEKYRDCERFKCPCPTCGTENIYDNVFDGSG
TDMEPSLYRCSNIDCKASPLTFTVQLSNKLIMDIRRFIKKYYDGWLICEEPTCRNRTRHLPLQF
SRTGPLCPACMKATLQPEYSDKSLYTQLCFYRYIFDAECALEKLTTDHEKDKLKKQFFTPKV
LQDYRKLKNTAEQFLSRSGYSEVNLSKLFAGCAVKS
Pol Delta
(SEQ ID NO: 61)
MDGKRRPGPGPGVPPKRARGGLWDDDDAPRPSQFEEDLALMEEMEAEHRLQEQEEEELQSV
LEGVADGQVPPSAIDPRWLRPTPPALDPQTEPLIFQQLEIDHYVGPAQPVPGGPPPSHGSVPVL
RAFGVTDEGFSVCCHIHGFAPYFYTPAPPGFGPEHMGDLQRELNLAISRDSRGGRELTGPAVL
AVELCSRESMFGYHGHGPSPFLRITVALPRLVAPARRLLEQGIRVAGLGTPSFAPYEANVDFEI
RFMVDTDIVGCNWLELPAGKYALRLKEKATQCQLEADVLWSDVVSHPPEGPWQRIAPLRVL
SFDIECAGRKGIFPEPERDPVIQICSLGLRWGEPEPFLRLALTLRPCAPILGAKVQSYEKEEDLL
QAWSTFIRIMDPDVITGYNIQNFDLPYLISRAQTLKVQTFPFLGRVAGLCSNIRDSSFQSKQTG
RRDTKVVSMVGRVQMDMLQVLLREYKLRSYTLNAVSFHFLGEQKEDVQHSIITDLQNGND
QTRRRLAVYCLKDAYLPLRLLERLMVLVNAVEMARVTGVPLSYLLSRGQQVKVVSQLLRQ
AMHEGLLMPVVKSEGGEDYTGATVIEPLKGYYDVPIATLDFSSLYPSIMMAHNLCYTTLLRP
GTAQKLGLTEDQFIRTPTGDEFVKTSVRKGLLPQILENLLSARKRAKAELAKETDPLRRQVLD
GRQLALKVSANSVYGFTGAQVGKLPCLEISQSVTGFGRQMIEKTKQLVESKYTVENGYSTSA
KVVYGDTDSVMCRFGVSSVAEAMALGREAADWVSGHFPSPIRLEFEKVYFPYLLISKKRYA
GLLFSSRPDAHDRMDCKGLEAVRRDNCPLVANLVTASLRRLLIDRDPEGAVAHAQDVISDLL
CNRIDISQLVITKELTRAASDYAGKQAHVELAERMRKRDPGSAPSLGDRVPYVIISAAKGVAA
YMKSEDPLFVLEHSLPIDTQYYLEQQLAKPLLRIFEPILGEGRAEAVLLRGDHTRCKTVLTGK
VGGLLAFAKRRNCCIGCRTVLSHQGAVCEFCQPRESELYQKEVSHLNALEERFSRLWTQCQR
CQGSLHEDVICTSRDCPIFYMRKKVRKDLEDQEQLLRRFGPPGPEAW
Pol Gamma
(SEQ ID NO: 62)
MSRLLWRKVAGATVGPGPVPAPGRWVSSSVPASDPSDGQRRRQQQQQQQQQQQQQPQQPQ
VLSSEGGQLRHNPLDIQMLSRGLHEQIFGQGGEMPGEAAVRRSVEHLQKHGLWGQPAVPLP
DVELRLPPLYGDNLDQHFRLLAQKQSLPYLEAANLLLQAQLPPKPPAWAWAEGWTRYGPEG
EAVPVAIPEERALVFDVEVCLAEGTCPTLAVAISPSAWYSWCSQRLVEERYSWTSQLSPADLI
PLEVPTGASSPTQRDWQEQLVVGHNVSFDRAHIREQYLIQGSRMRFLDTMSMHMAISGLSSF
QRSLWIAAKQGKHKVQPPTKQGQKSQRKARRGPAISSWDWLDISSVNSLAEVHRLYVGGPP
LEKEPRELFVKGTMKDIRENFQDLMQYCAQDVWATHEVFQQQLPLFLERCPHPVTLAGMLE
MGVSYLPVNQNWERYLAEAQGTYEELQREMKKSLMDLANDACQLLSGERYKEDPWLWDL
EWDLQEFKQKKAKKVKKEPATASKLPIEGAGAPGDPMDQEDLGPCSEEEEFQQDVMARACL
QKLKGTTELLPKRPQHLPGHPGWYRKLCPRLDDPAWTPGPSLLSLQMRVTPKLMALTWDGF
PLHYSERHGWGYLVPGRRDNLAKLPTGTTLESAGVVCPYRAIESLYRKHCLEQGKQQLMPQ
EAGLAEEFLLTDNSAIWQTVEELDYLEVEAEAKMENLRAAVPGQPLALTARGGPKDTQPSY
HHGNGPYNDVDIPGCWFFKLPHKDGNSCNVGSPFAKDFLPKMEDGTLQAGPGGASGPRALE
INKMISFWRNAHKRISSQMVVWLPRSALPRAVIRHPDYDEEGLYGAILPQVVTAGTITRRAVE
PTWLTASNARPDRVGSELKAMVQAPPGYTLVGADVDSQELWIAAVLGDAHFAGMHGCTAF
GWMTLQGRKSRGTDLHSKTATTVGISREHAKIFNYGRIYGAGQPFAERLLMQFNHRLTQQE
AAEKAQQMYAATKGLRWYRLSDEGEWLVRELNLPVDRTEGGWISLQDLRKVQRETARKSQ
WKKWEVVAERAWKGGTESEMFNKLESIATSDIPRTPVLGCCISRALEPSAVQEEFMTSRVNW
VVQSSAVDYLHLMLVAMKWLFEEFAIDGRFCISIHDEVRYLVREEDRYRAALALQITNLLTR
CMFAYKLGLNDLPQSVAFFSAVDIDRCLRKEVTMDCKTPSNPTGMERRYGIPQGEALDIYQII
ELTKGSLEKRSQPGP
Pol Nu
(SEQ ID NO: 63)
MENYEALVGFDLCNTPLSSVAQKIMSAMHSGDLVDSKTWGKSTETMEVINKSSVKYSVQLE
DRKTQSPEKKDLKSLRSQTSRGSAKLSPQSFSVRLTDQLSADQKQKSISSLTLSSCLIPQYNQE
ASVLQKKGHKRKHFLMENINNENKGSINLKRKHITYNNLSEKTSKQMALEEDTDDAEGYLN
SGNSGALKKHFCDIRHLDDWAKSQLIEMLKQAAALVITVMYTDGSTQLGADQTPVSSVRGI
VVLVKRQAEGGHGCPDAPACGPVLEGFVSDDPCIYIQIEHSAIWDQEQEAHQQFARNVLFQT
MKCKCPVICFNAKDFVRIVLQFFGNDGSWKHVADFIGLDPRIAAWLIDPSDATPSFEDLVEKY
CEKSITVKVNSTYGNSSRNIVNQNVRENLKTLYRLTMDLCSKLKDYGLWQLFRTLELPLIPIL
AVMESHAIQVNKEEMEKTSALLGARLKELEQEAHFVAGERFLITSNNQLREILFGKLKLHLLS
QRNSLPRTGLQKYPSTSEAVLNALRDLHPLPKIILEYRQVHKIKSTFVDGLLACMKKGSISST
WNQTGTVTGRLSAKHPNIQGISKHPIQITTPKNFKGKEDKILTISPRAMFVSSKGHTFLAADFS
QIELRILTHLSGDPELLKLFQESERDDVFSTLTSQWKDVPVEQVTHADREQTKKVVYAVVYG
AGKERLAACLGVPIQEAAQFLESFLQKYKKIKDFARAAIAQCHQTGCVVSIMGRRRPLPRIHA
HDQQLRAQAERQAVNFVVQGSAADLCKLAMIHVFTAVAASHTLTARLVAQIHDELLFEVED
PQIPECAALVRRTMESLEQVQALELQLQVPLKVSLSAGRSWGHLVPLQEAWGPPPGPCRTES
PSNSLAAPGSPASTQPPPLHFSPSFCL
Rev1
(SEQ ID NO: 64)
MRRGGWRKRAENDGWETWGGYMAAKVQKLEEQFRSDAAMQKDGTSSTIFSGVAIYVNGY
TDPSAEELRKLMMLHGGQYHVYYSRSKTTHIIATNLPNAKIKELKGEKVIRPEWIVESIKAGR
LLSYIPYQLYTKQSSVQKGLSFNPVCRPEDPLPGPSNIAKQLNNRVNHIVKKIETENEVKVNG
MNSWNEEDENNDFSFVDLEQTSPGRKQNGIPHPRGSTAIFNGHTPSSNGALKTQDCLVPMVN
SVASRLSPAFSQEEDKAEKSSTDFRDCTLQQLQQSTRNTDALRNPHRTNSFSLSPLHSNTKING
AHHSTVQGPSSTKSTSSVSTFSKAAPSVPSKPSDCNFISNFYSHSRLHHISMWKCELTEFVNTL
QRQSNGIFPGREKLKKMKTGRSALVVTDTGDMSVLNSPRHQSCIMHVDMDCFFVSVGIRNR
PDLKGKPVAVTSNRGTGRAPLRPGANPQLEWQYYQNKILKGKAADIPDSSLWENPDSAQAN
GIDSVLSRAEIASCSYEARQLGIKNGMFFGHAKQLCPNLQAVPYDFHAYKEVAQTLYETLAS
YTHNIEAVSCDEALVDITEILAETKLTPDEFANAVRMEIKDQTKCAASVGIGSNILLARMATR
KAKPDGQYHLKPEEVDDFIRGQLVTNLPGVGHSMESKLASLGIKTCGDLQYMTMAKLQKEF
GPKTGQMLYRFCRGLDDRPVRTEKERKSVSAEINYGIRFTQPKEAEAFLLSLSEEIQRRLEAT
GMKGKRLTLKIMVRKPGAPVETAKFGGHGICDNIARTVTLDQATDNAKIIGKAMLNMFHTM
KLNISDMRGVGIHVNQLVPTNLNPSTCPSRPSVQSSHFPSGSYSVRDVFQVQKAKKSTEEEHK
EVFRAAVDLEISSASRTCTFLPPFPAHLPTSPDTNKAESSGKWNGLHTPVSVQSRLNLSIEVPSP
SQLDQSVLEALPPDLREQVEQVCAVQQAESHGDKKKEPVNGCNTGILPQPVGTVLLQIPEPQ
ESNSDAGINLIALPAFSQVDPEVFAALPAELQRELKAAYDQRQRQGENSTHQQSASASVPKNP
LLHLKAAVKEKKRNKKKKTIGSPKRIQSPLNNKLLNSPAKTLPGACGSPQKLIDGFLKHEGPP
AEKPLEELSASTSGVPGLSSLQSDPAGCVRPPAPNLAGAVEFNDVKTLLREWITTISDPMEEDI
LQVVKYCTDLIEEKDLEKLDLVIKYMKRLMQQSVESVWNMAFDFILDNVQVVLQQTYGSTL
KVT

### Base Excision Enzymes (BEE)

A base excision enzyme, or BEE, refers to a protein that is capable of removing a base (e.g., A, T, C, G, or U) from a nucleic acid molecule (e.g., DNA or RNA). In some embodiments, a BEE is capable of removing a cytosine from DNA. In some embodiments, a BEE is capable of removing a thymine from DNA. Exemplary BEEs include, without limitation UDG Tyr147Ala, and UDG Asn204Asp as described in Sang et al., “A Unique Uracil-DNA binding protein of the uracil DNA glycosylase superfamily,” Nucleic Acids Research, Vol. 43, No. 17 2015; the entire contents of which are hereby incorporated by reference.

In some embodiments, the base excision enzyme (BEE) is a cytosine, thymine, adenine, guanine, or uracil base excision enzyme. In some embodiments, the base excision enzyme (BEE) is a cytosine base excision enzyme. In some embodiments, the BEE is a thymine base excision enzyme. In some embodiments, the base excision enzyme comprises an amino acid sequence that is at least 75%, 80%, 85%, 90%, 95%, 96%, 97%, 98%, 99%, or 99.5% identical to a naturally-occurring BEE. In some embodiments, the base excision enzyme comprises an amino acid sequence that is at least 75%, 80%, 85%, 90%, 95%, 96%, 97%, 98%, 99%, or 99.5% identical to any of the BEEs provided herein, e.g., UDG (Tyr147Ala), or UDG (Asn204Asp), below. In some embodiments, the base excision enzyme comprises an amino acid sequence that is at least 75%, 80%, 85%, 90%, 95%, 96%, 97%, 98%, 99%, or 99.5% identical to any one of SEQ ID NOs: 65-66. In some embodiments, the base excision enzyme comprises the amino acid sequence of any one of SEQ ID NOs: 65-66. In some embodiments, the base excision enzyme has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to any BEE provided herein, such as any one of SEQ ID NOs: 65-66.

The disclosure also provides fragments of BEEs, such as truncations of any of the BEEs provided herein. In some embodiments, the BEE is an N-terminal truncation, where one or more amino acids are absent from the N-terminus of the BEE. In some embodiments, the BEE is absent 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the N-terminus of the BEE. For example, the N-terminal truncation of the BEE may be an N-terminal truncation of any BEE provided herein, such as any one of the BEEs provided in any one of SEQ ID NOs: 65-66. In some embodiments, the BEE is a C-terminal truncation, where one or more amino acids are absent from the C-terminus of the BEE. In some embodiments, the BEE is absent 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the C-terminus of the BEE. For example, the C-terminal truncation of the BEE may be a C-terminal truncation of any BEE provided herein, such as any one of the BEEs provided in any one of SEQ ID NOs: 65-66.

It should be appreciated that other BEEs would be apparent to the skilled artisan and are within the scope of this disclosure. For example BEEs have been described previously in Sang et al., “A Unique Uracil-DNA binding protein of the uracil DNA glycosylase superfamily,” Nucleic Acids Research, Vol. 43, No. 17 2015; the entire contents of which are hereby incorporated by reference.

UDG (Tyr147Ala)-The mutated residue is indicated
by bold and underlining.
(SEQ ID NO: 65)
MIGQKTLYSFFSPSPARKRHAPSPEPAVQGTGVAGVPEESGDAAAIPAK
KAPAGQEEPGTPPSSPLSAEQLDRIQRNKAAALLRLAARNVPVGFGESW
KKHLSGEFGKPYFIKLMGFVAEERKHYTVYPPPHQVFTWTQMCDIKDVK
VVILGQDPAHGPNQAHGLCFSVQRPVPPPPSLENIYKELSTDIEDFVHP
GHGDLSGWAKQGVLLLNAVLTVRAHQANSHKERGWEQFTDAVVSWLNQN
SNGLVFLLWGSYAQKKGSAIDRKRHHVLQTAHPSPLSVYRGFFGCRHFS
KTNELLQKSGKKPIDWKEL
UDG (Asn204Asp)-The mutated residue is indicated
by bold and underlining.
(SEQ ID NO: 66)
MIGQKTLYSFFSPSPARKRHAPSPEPAVQGTGVAGVPEESGDAAAIPAK
KAPAGQEEPGTPPSSPLSAEQLDRIQRNKAAALLRLAARNVPVGFGESW
KKHLSGEFGKPYFIKLMGFVAEERKHYTVYPPPHQVFTWTQMCDIKDVK
VVILGQDPYHGPNQAHGLCFSVQRPVPPPPSLENIYKELSTDIEDFVHP
GHGDLSGWAKQGVLLLDAVLTVRAHQANSHKERGWEQFTDAVVSWLNQN
SNGLVFLLWGSYAQKKGSAIDRKRHHVLQTAHPSPLSVYRGFFGCRHFS
KTNELLQKSGKKPIDWKEL

### Deaminase Domains

In some embodiments, any of the fusion proteins or base editors provided herein comprise a cytidine deaminase domain. In some embodiments, the cytidine deaminase domain can catalyze a C to U base change. In some embodiments, the cytidine deaminase domain is an apolipoprotein B mRNA-editing complex (APOBEC) family deaminase. In some embodiments, the cytidine deaminase domain is an APOBEC1 deaminase. In some embodiments, the cytidine deaminase domain is a rat APOBEC1 deaminase (rAPOBEC1). In some embodiments, the cytidine deaminase a variant of rAPOBEC1, such as the R126E+R132E double mutant known as EE deaminase. In some embodiments, the cytidine deaminase domain is a YEE, YE1 or YE2 variant of rAPOBEC1. See Kim et al. Nature Biotechnology (2018).

In some embodiments, the cytidine deaminase domain is an APOBEC2 deaminase. In some embodiments, the cytidine deaminase domain is an APOBEC3 deaminase. In some embodiments, the cytidine deaminase domain is an APOBEC3A deaminase. In some embodiments, the cytidine deaminase domain is an APOBEC3B deaminase. In some embodiments, the cytidine deaminase domain is an APOBEC3C deaminase. In some embodiments, the cytidine deaminase domain is an APOBEC3D deaminase. In some embodiments, the cytidine deaminase domain is an APOBEC3E deaminase. In some embodiments, the cytidine deaminase domain is an APOBEC3F deaminase. In some embodiments, the cytidine deaminase domain is an APOBEC3G deaminase. In some embodiments, the cytidine deaminase domain is an APOBEC3H deaminase. In some embodiments, the cytidine deaminase domain is an APOBEC4 deaminase. In some embodiments, the cytidine deaminase domain is an activation-induced deaminase (AID). In some embodiments, the cytidine deaminase domain is a vertebrate deaminase. In some embodiments, the cytidine deaminase domain is an invertebrate deaminase. In some embodiments, the cytidine deaminase domain is a human, chimpanzee, gorilla, monkey, cow, dog, rat, or mouse deaminase. In some embodiments, the cytidine deaminase domain is a human deaminase. In some embodiments, the cytidine deaminase domain is a rat deaminase, e.g., rAPOBEC1. In some embodiments, the cytidine deaminase domain is a Petromyzon marinus cytidine deaminase 1 (pmCDA1). In some embodiments, the cytidine deaminase domain is a human APOBEC3G (SEQ ID NO: 77). In some embodiments, the cytidine deaminase domain is a fragment of the human APOBEC3G (SEQ ID NO: 100). In some embodiments, the cytidine deaminase domain is a human APOBEC3G variant comprising a D316R_D317R mutation (SEQ ID NO: 99). In some embodiments, the cytidine deaminase domain is a frantment of the human APOBEC3G and comprising mutations corresponding to the D316R_D317R mutations in SEQ ID NO: 77 (SEQ ID NO: 101).

In some embodiments, the cytidine deaminase domain is a rat APOBEC3A, such as a human APOBEC3A deaminase. In some embodiments, the cytidine deaminase domain is an evolved human APOBEC3A (eA3A) deaminase (SEQ ID NO: 85). In some embodiments, the cytidine deaminase domain is aAPOBEC3A (eA3A) deaminase comprising a T31A mutation in SEQ ID NO: 93. See Gehrke et al. Nature Biotechnology (2019).

In some embodiments, the cytidine deaminase domain is an ancestrally reconstructed rAPOBEC1 node 689²⁹ (Anc689). See Koblan, L. W. et al. Nature Biotechnology 36, 843-846 (2018), which is incorporated by reference herein. In some embodiments, the cytidine deaminase domain is at least 80%, at least 85%, at least 90%, at least 92%, at least 95%, at least 96%, at least 97%, at least 98%, at least 99%, or at least 99.5% identical to a naturally-occurring cytidine deaminase. In some embodiments, the cytidine deaminase domain is at least 80%, at least 85%, at least 90%, at least 92%, at least 95%, at least 96%, at least 97%, at least 98%, at least 99%, or at least 99.5% identical to any of the cytidine deaminases provided herein. In some embodiments, the cytidine deaminase domain is at least 80%, at least 85%, at least 90%, at least 92%, at least 95%, at least 96%, at least 97%, at least 98%, at least 99%, or at least 99.5% identical to the deaminase domain of any one of SEQ ID NOs: 67-101. In some embodiments, the nucleic acid editing domain comprises the amino acid sequence of any one of SEQ ID NOs: 67-101. In some embodiments, the cytidine deaminase domain has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to any cytidine deaminase domain provided herein, such as any one of SEQ ID NOs: 67-101.

The disclosure also provides fragments of cytidine deaminase domains, such as truncations of any of the cytidine deaminase domains provided herein. In some embodiments, the cytidine deaminase domain is an N-terminal truncation, where one or more amino acids are absent from the N-terminus of the cytidine deaminase domain. In some embodiments, the cytidine deaminase domain is absent 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the N-terminus of the cytidine deaminase domain. For example, the N-terminal truncation of the cytidine deaminase domain may be an N-terminal truncation of any cytidine deaminase domain provided herein, such as any one of the cytidine deaminase domains provided in any one of SEQ ID NOs: 67-101. In some embodiments, the cytidine deaminase domain is a C-terminal truncation, where one or more amino acids are absent from the C-terminus of the cytidine deaminase domain. In some embodiments, the cytidine deaminase domain is absent 1, 2, 3, 4, 5, 6,7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, or 50 amino acids from the C-terminus of the cytidine deaminase domain. For example, the C-terminal truncation of the cytidine deaminase domain may be a C-terminal truncation of any cytidine deaminase domain provided herein, such as any one of the cytidine deaminase domains provided in any one of SEQ ID NOs: 67-101.

Some exemplary cytidine deaminase domains include, without limitation, those provided below. It should be understood that, in some embodiments, the active domain of the respective sequence can be used, e.g., the domain without a localizing signal (nuclear localization sequence, without nuclear export signal, cytoplasmic localizing signal).

Human AID:
(SEQ ID NO: 67)
MDSLLMNRRKFLYQFKNVRWAKGRRETYLCYVVKRRDSATSFSLDFGYLRN
KNGCHVELLFLRYISDWDLDPGRCYRVTWFTSWSPCYDCARHVADFLRGNPNLSLR
IFTARLYFCEDRKAEPEGLRRLHRAGVQIAIMTFKDYFYCWNTFVENHERTFKAWEG
LHENSVRLSRQLRRILLPLYEVDDLRDAFRTLGL
(underline: nuclear localization sequence; double underline: nuclear export signal)
Mouse AID:
(SEQ ID NO: 68)
MDSLLMKQKKFLYHFKNVRWAKGRHETYLCYVVKRRDSATSCSLDFGHLR
NKSGCHVELLFLRYISDWDLDPGRCYRVTWFTSWSPCYDCARHVAEFLRWNPNLSL
RIFTARLYFCEDRKAEPEGLRRLHRAGVQIGIMTFKDYFYCWNTFVENRERTFKAWE
GLHENSVRLTRQLRRILLPLYEVDDLRDAFRMLGE
(underline: nuclear localization sequence; double underline: nuclear export signal)
Dog AID:
(SEQ ID NO: 69)
MDSLLMKQRKFLYHFKNVRWAKGRHETYLCYVVKRRDSATSFSLDFGHLR
NKSGCHVELLFLRYISDWDLDPGRCYRVTWFTSWSPCYDCARHVADFLRGYPNLSL
RIFAARLYFCEDRKAEPEGLRRLHRAGVQIAIMTFKDYFYCWNTFVENREKTFKAWE
GLHENSVRLSRQLRRILLPLYEVDDLRDAFRTLGL
(underline: nuclear localization sequence; double underline: nuclear export signal)
Bovine AID:
(SEQ ID NO: 70)
MDSLLKKQRQFLYQFKNVRWAKGRHETYLCYVVKRRDSPTSFSLDFGHLRN
KAGCHVELLFLRYISDWDLDPGRCYRVTWFTSWSPCYDCARHVADFLRGYPNLSLR
IFTARLYFCDKERKAEPEGLRRLHRAGVQIAIMTFKDYFYCWNTFVENHERTFKAWE
GLHENSVRLSRQLRRILLPLYEVDDLRDAFRTLGL
(underline: nuclear localization sequence; double underline: nuclear export signal)
Rat AID
MAVGSKPKAALVGPHWERERIWCFLCSTGLGTQQTGQTSRWLRPAATQDPVSPPRS
LLMKQRKFLYHFKNVRWAKGRHETYLCYVVKRRDSATSFSLDFGYLRNKSGCHVE
LLFLRYISDWDLDPGRCYRVTWFTSWSPCYDCARHVADFLRGNPNLSLRIFTARLTG
WGALPAGLMSPARPSDYFYCWNTFVENHERTFKAWEGLHENSVRLSRRLRRILLPL
YEVDDLRDAFRTLGL (SEQ ID NO: 71)
(underline: nuclear localization sequence; double underline: nuclear export signal)
Mouse APOBEC-3:
(SEQ ID NO: 72)
MGPFCLGCSHRKCYSPIRNLISQETFKFHFKNLGYAKGRKDTFLCYEVTRKDC
DSPVSLHHGVFKNKDNIHAEICFLYWFHDKVLKVLSPREEFKITWYMSWSPCFECAEQI
VRFLATHHNLSLDIFSSRLYNVQDPETQQNLCRLVQEGAQVAAMDLYEFKKCWKKF
VDNGGRRFRPWKRLLTNFRYQDSKLQEILRPCYIPVPSSSSSTLSNICLTKGLPETRFC
VEGRRMDPLSEEEFYSQFYNQRVKHLCYYHRMKPYLCYQLEQFNGQAPLKGCLLSE
KGKQHAEILFLDKIRSMELSQVTITCYLTWSPCPNCAWQLAAFKRDRPDLILHIYTSRLY
FHWKRPFQKGLCSLWQSGILVDVMDLPQFTDCWTNFVNPKRPFWPWKGLEIISRRT
QRRLRRIKESWGLQDLVNDFGNLQLGPPMS
(italic: nucleic acid editing domain)
Rat APOBEC-3:
(SEQ ID NO: 73)
MGPFCLGCSHRKCYSPIRNLISQETFKFHFKNLRYAIDRKDTFLCYEVTRKDC
DSPVSLHHGVFKNKDNIHAEICFLYWFHDKVLKVLSPREEFKITWYMSWSPCFECAEQV
LRFLATHHNLSLDIFSSRLYNIRDPENQQNLCRLVQEGAQVAAMDLYEFKKCWKKF
VDNGGRRFRPWKKLLTNFRYQDSKLQEILRPCYIPVPSSSSSTLSNICLTKGLPETRFC
VERRRVHLLSEEEFYSQFYNQRVKHLCYYHGVKPYLCYQLEQFNGQAPLKGCLLSE
KGKQHAEILFLDKIRSMELSQVIITCYLTWSPCPNCAWQLAAFKRDRPDLILHIYTSRLY
FHWKRPFQKGLCSLWQSGILVDVMDLPQFTDCWTNFVNPKRPFWPWKGLEIISRRT
QRRLHRIKESWGLQDLVNDFGNLQLGPPMS
(italic: nucleic acid editing domain)
Rhesus macaque APOBEC-3G:
(SEQ ID NO: 74)
MVEPMDPRTFVSNENNRPILSGLNTVWLCCEVKTKDPSGPPLDAKIFQGKVY
SKAKYHPEMRFLRWFHKWRQLHHDQEYKVTWYVSWSPCTRCANSVATFLAKDPKVTL
TIFVARLYYFWKPDYQQALRILCQKRGGPHATMKIMNYNEFQDCWNKFVDGRGKP
FKPRNNLPKHYTLLQATLGELLRHLMDPGTFTSNFNNKPWVSGQHETYLCYKVERL
HNDTWVPLNQHRGFLRNQAPNIHGFPKGRHAELCFLDLIPFWKLDGQQYRVTCFTSWS
PCFSCAQEMAKFISNNEHVSLCIFAARIYDDQGRYQEGLRALHRDGAKIAMMNYSEF
EYCWDTFVDRQGRPFQPWDGLDEHSQALSGRLRAI
(italic: nucleic acid editing domain; underline: cytoplasmic localization signal)
Chimpanzee APOBEC-3G:
(SEQ ID NO: 75)
MKPHFRNPVERMYQDTESDNFYNRPILSHRNTVWLCYEVKTKGPSRPPLDAK
IFRGQVYSKLKYHPEMRFFHWFSKWRKLHRDQEYEVTWYISWSPCTKCTRDVATFLAE
DPKVTLTIFVARLYYFWDPDYQEALRSLCQKRDGPRATMKIMNYDEFQHCWSKFV
YSQRELFEPWNNLPKYYILLHIMLGEILRHSMDPPTFTSNFNNELWVRGRHETYLCY
EVERLHNDTWVLLNQRRGFLCNQAPHKHGFLEGRHAELCFLDVIPFWKLDLHQDYRV
TCFTSWSPCFSCAQEMAKFISNNKHVSLCIFAARIYDDQGRCQEGLRTLAKAGAKISI
MTYSEFKHCWDTFVDHQGCPFQPWDGLEEHSQALSGRLRAILQNQGN
(italic: nucleic acid editing domain; underline: cytoplasmic localization signal)
Green monkey APOBEC-3G:
(SEQ ID NO: 76)
MNPQIRNMVEQMEPDIFVYYENNRPILSGRNTVWLCYEVKTKDPSGPPLDAN
IFQGKLYPEAKDHPEMKFLHWFRKWRQLHRDQEYEVTWYVSWSPCTRCANSVATFLA
EDPKVTLTIFVARLYYFWKPDYQQALRILCQERGGPHATMKIMNYNEFQHCWNEFV
DGQGKPFKPRKNLPKHYTLLHATLGELLRHVMDPGTFTSNFNNKPWVSGQRETYLC
YKVERSHNDTWVLLNQHRGFLRNQAPDRHGFPKGRHAELCFLDLIPFWKLDDQQYR
VTCFTSWSPCFSCAQKMAKFISNNKHVSLCIFAARIYDDQGRCQEGLRTLHRDGAKIA
VMNYSEFEYCWDTFVDRQGRPFQPWDGLDEHSQALSGRLRAI
(italic: nucleic acid editing domain; underline: cytoplasmic localization signal)
Human APOBEC-3G:
(SEQ ID NO: 77)
MKPHFRNTVERMYRDTESYNFYNRPILSRRNTVWLCYEVKTKGPSRPPLDAK
IFRGQVYSELKYHPEMRFFHWFSKWRKLHRDQEYEVTWYISWSPCTKCTRDMATFLAE
DPKVTLTIFVARLYYFWDPDYQEALRSLCQKRDGPRATMKIMNYDEFQHCWSKFV
YSQRELFEPWNNLPKYYILLHIMLGEILRHSMDPPTFTFNFNNEPWVRGRHETYLCYE
VERMHNDTWVLLNQRRGFLCNQAPHKHGFLEGRHAELCFLDVIPFWKLDLDQDYRV
TCFTSWSPCFSCAQEMAKFISKNKHVSLCIFTARIYDDQGRCQEGLRTLAEAGAKISIM
TYSEFKHCWDTFVDHQGCPFQPWDGLDEHSQDLSGRLRAILQNQEN
(italic: nucleic acid editing domain; underline: cytoplasmic localization signal)
Human APOBEC-3F:
(SEQ ID NO: 78)
MKPHFRNTVERMYRDTFSYNFYNRPILSRRNTVWLCYEVKTKGPSRPRLDAK
IFRGQVYSQPEHHAEMCFLSWFCGNQLPAYKCFQITWFVSWTPCPDCVAKLAEFLAEH
PNVTLTISAARLYYYWERDYRRALCRLSQAGARVKIMDDEEFAYCWENFVYSEGQP
FMPWYKFDDNYAFLHRTLKEILRNPMEAMYPHIFYFHFKNLRKAYGRNESWLCFTM
EVVKHHSPVSWKRGVFRNQVDPETHCHAERCFLSWFCDDILSPNTNYEVTWYTSWSPC
PECAGEVAEFLARHSNVNLTIFTARLYYFWDTDYQEGLRSLSQEGASVEIMGYKDFK
YCWENFVYNDDEPFKPWKGLKYNFLFLDSKLQEILE
(italic: nucleic acid editing domain)
Human APOBEC-3B:
(SEQ ID NO: 79)
MNPQIRNPMERMYRDTFYDNFENEPILYGRSYTWLCYEVKIKRGRSNLLWDT
GVFRGQVYFKPQYHAEMCFLSWFCGNQLPAYKCFQITWFVSWTPCPDCVAKLAEFLS
EHPNVTLTISAARLYYYWERDYRRALCRLSQAGARVTIMDYEEFAYCWENFVYNEG
QQFMPWYKFDENYAFLHRTLKEILRYLMDPDTFTFNFNNDPLVLRRRQTYLCYEVE
RLDNGTWVLMDQHMGFLCNEAKNLLCGFYGRHAELRFLDLVPSLQLDPAQIYRVTWF
ISWSPCFSWGCAGEVRAFLQENTHVRLRIFAARIYDYDPLYKEALQMLRDAGAQVSI
MTYDEFEYCWDTFVYRQGCPFQPWDGLEEHSQALSGRLRAILQNQGN
(italic: nucleic acid editing domain)
Rat APOBEC3:
(SEQ ID NO: 80)
MQPQGLGPNAGMGPVCLGCSHRRPYSPIRNPLKKLYQQTFYFHFKNVRYAW
GRKNNFLCYEVNGMDCALPVPLRQGVFRKQGHIHAELCFIYWFHDKVLRVLSPMEE
FKVTWYMSWSPCSKCAEQVARFLAAHRNLSLAIFSSRLYYYLRNPNYQQKLCRLIQ
EGVHVAAMDLPEFKKCWNKFVDNDGQPFRPWMRLRINFSFYDCKLQEIFSRMNLLR
EDVFYLQFNNSHRVKPVQNRYYRRKSYLCYQLERANGQEPLKGYLLYKKGEQHVEI
LFLEKMRSMELSQVRITCYLTWSPCPNCARQLAAFKKDHPDLILRIYTSRLYFYWRK
KFQKGLCTLWRSGIHVDVMDLPQFADCWTNFVNPQRPFRPWNELEKNSWRIQRRLR
RIKESWGL
Bovine APOBEC-3B:
(SEQ ID NO: 81)
DGWEVAFRSGTVLKAGVLGVSMTEGWAGSGHPGQGACVWTPGTRNTMNL
LREVLFKQQFGNQPRVPAPYYRRKTYLCYQLKQRNDLTLDRGCFRNKKQRHAEIRFI
DKINSLDLNPSQSYKIICYITWSPCPNCANELVNFITRNNHLKLEIFASRLYFHWIKSFK
MGLQDLQNAGISVAVMTHTEFEDCWEQFVDNQSRPFQPWDKLEQYSASIRRRLQRI
LTAPI
Chimpanzee APOBEC-3B:
(SEQ ID NO: 82)
MNPQIRNPMEWMYQRTFYYNFENEPILYGRSYTWLCYEVKIRRGHSNLLWDTGVFR
GQMYSQPEHHAEMCFLSWFCGNQLSAYKCFQITWFVSWTPCPDCVAKLAKFLAEH
PNVTLTISAARLYYYWERDYRRALCRLSQAGARVKIMDDEEFAYCWENFVYNEGQP
FMPWYKFDDNYAFLHRTLKEIIRHLMDPDTFTFNFNNDPLVLRRHQTYLCYEVERLD
NGTWVLMDQHMGFLCNEAKNLLCGFYGRHAELRFLDLVPSLQLDPAQIYRVTWFIS
WSPCFSWGCAGQVRAFLQENTHVRLRIFAARIYDYDPLYKEALQMLRDAGAQVSIM
TYDEFEYCWDTFVYRQGCPFQPWDGLEEHSQALSGRLRAILQVRASSLCMVPHRPPP
PPQSPGPCLPLCSEPPLGSLLPTGRPAPSLPFLLTASFSFPPPASLPPLPSLSLSPGHLPVP
SFHSLTSCSIQPPCSSRIRETEGWASVSKEGRDLG
Human APOBEC-3C:
(SEQ ID NO: 83)
MNPQIRNPMKAMYPGTFYFQFKNLWEANDRNETWLCFTVEGIKRRSVVSWK
TGVFRNQVDSETHCHAERCFLSWFCDDILSPNTKYQVTWYTSWSPCPDCAGEVAEFLA
RHSNVNLTIFTARLYYFQYPCYQEGLRSLSQEGVAVEIMDYEDFKYCWENFVYNDN
EPFKPWKGLKTNFRLLKRRLRESLQ
(italic: nucleic acid editing domain)
Gorilla APOBEC3C
(SEQ ID NO: 84)
MNPQIRNPMKAMYPGTFYFQFKNLWEANDRNETWLCFTVEGIKRRSVVSWKTGVF
RNQVDSETHCHAERCFLSWFCDDILSPNTNYQVTWYTSWSPCPECAGEVAEFLARHSN
VNLTIFTARLYYFQDTDYQEGLRSLSQEGVAVKIMDYKDFKYCWENFVYNDDEPFK
PWKGLKYNFRFLKRRLQEILE
(italic: nucleic acid editing domain)
Human APOBEC-3A:
(SEQ ID NO: 85)
MEASPASGPRHLMDPHIFTSNFNNGIGRHKTYLCYEVERLDNGTSVKMDQHR
GFLHNQAKNLLCGFYGRHAELRFLDLVPSLQLDPAQIYRVTWFISWSPCFSWGCAGEVR
AFLQENTHVRLRIFAARIYDYDPLYKEALQMLRDAGAQVSIMTYDEFKHCWDTFVD
HQGCPFQPWDGLDEHSQALSGRLRAILQNQGN
(italic: nucleic acid editing domain)
Rhesus macaque APOBEC-3A:
(SEQ ID NO: 86)
MDGSPASRPRHLMDPNTFTFNFNNDLSVRGRHQTYLCYEVERLDNGTWVPMDERR
GFLCNKAKNVPCGDYGCHVELRFLCEVPSWQLDPAQTYRVTWFISWSPCFRRGCAGQ
VRVFLQENKHVRLRIFAARIYDYDPLYQEALRTLRDAGAQVSIMTYEEFKHCWDTF
VDRQGRPFQPWDGLDEHSQALSGRLRAILQNQGN
(italic: nucleic acid editing domain)
Bovine APOBEC-3A:
(SEQ ID NO: 87)
MDEYTFTENFNNQGWPSKTYLCYEMERLDGDATIPLDEYKGFVRNKGLDQPEKPCH
AELYFLGKIHSWNLDRNQHYRLTCFISWSPCYDCAQKLTTFLKENHHISLHILASRIYTH
NRFGCHQSGLCELQAAGARITIMTFEDFKHCWETFVDHKGKPFQPWEGLNVKSQAL
CTELQAILKTQQN
(italic: nucleic acid editing domain)
Human APOBEC-3H:
(SEQ ID NO: 88)
MALLTAETFRLQFNNKRRLRRPYYPRKALLCYQLTPQNGSTPTRGYFENKKK
CHAEICFINEIKSMGLDETQCYQVTCYLTWSPCSSCAWELVDFIKAHDHLNLGIFASRLY
YHWCKPQQKGLRLLCGSQVPVEVMGFPKFADCWENFVDHEKPLSFNPYKMLEELD
KNSRAIKRRLERIKIPGVRAQGRYMDILCDAEV
(italic: nucleic acid editing domain)
Rhesus macaque APOBEC-3H:
(SEQ ID NO: 89)
MALLTAKTFSLQFNNKRRVNKPYYPRKALLCYQLTPQNGSTPTRGHLKNKK
KDHAEIRFINKIKSMGLDETQCYQVTCYLTWSPCPSCAGELVDFIKAHRHLNLRIFAS
RLYYHWRPNYQEGLLLLCGSQVPVEVMGLPEFTDCWENFVDHKEPPSFNPSEKLEE
LDKNSQAIKRRLERIKSRSVDVLENGLRSLQLGPVTPSSSIRNSR
Human APOBEC-3D:
(SEQ ID NO: 90)
MNPQIRNPMERMYRDTFYDNFENEPILYGRSYTWLCYEVKIKRGRSNLLWDTGVFR
GPVLPKRQSNHRQEVYFRFENHAEMCFLSWFCGNRLPANRRFQITWFVSWNPCLPCVV
KVTKFLAEHPNVTLTISAARLYYYRDRDWRWVLLRLHKAGARVKIMDYEDFAYCW
ENFVCNEGQPFMPWYKFDDNYASLHRTLKEILRNPMEAMYPHIFYFHFKNLLKACG
RNESWLCFTMEVTKHHSAVFRKRGVFRNQVDPETHCHAERCFLSWFCDDILSPNTNY
EVTWYTSWSPCPECAGEVAEFLARHSNVNLTIFTARLCYFWDTDYQEGLCSLSQEGAS
VKIMGYKDFVSCWKNFVYSDDEPFKPWKGLQTNFRLLKRRLREILQ
(italic: nucleic acid editing domain)
Human APOBEC-1:
(SEQ ID NO: 91)
MTSEKGPSTGDPTLRRRIEPWEFDVFYDPRELRKEACLLYEIKWGMSRKIWRS
SGKNTTNHVEVNFIKKFTSERDFHPSMSCSITWFLSWSPCWECSQAIREFLSRHPGVT
LVIYVARLFWHMDQQNRQGLRDLVNSGVTIQIMRASEYYHCWRNFVNYPPGDEAH
WPQYPPLWMMLYALELHCIILSLPPCLKISRRWQNHLTFFRLHLQNCHYQTIPPHILL
ATGLIHPSVAWR
Mouse APOBEC-1:
(SEQ ID NO: 92)
MSSETGPVAVDPTLRRRIEPHEFEVFFDPRELRKETCLLYEINWGGRHSVWRH
TSQNTSNHVEVNFLEKFTTERYFRPNTRCSITWFLSWSPCGECSRAITEFLSRHPYVTL
FIYIARLYHHTDQRNRQGLRDLISSGVTIQIMTEQEYCYCWRNFVNYPPSNEAYWPR
YPHLWVKLYVLELYCIILGLPPCLKILRRKQPQLTFFTITLQTCHYQRIPPHLLWATGL
K
Rat APOBEC-1:
(SEQ ID NO: 93)
MSSETGPVAVDPTLRRRIEPHEFEVFFDPRELRKETCLLYEINWGGRHSIWRH
TSQNTNKHVEVNFIEKFTTERYFCPNTRCSITWFLSWSPCGECSRAITEFLSRYPHVTL
FIYIARLYHHADPRNRQGLRDLISSGVTIQIMTEQESGYCWRNFVNYSPSNEAHWPR
YPHLWVRLYVLELYCIILGLPPCLNILRRKQPQLTFFTIALQSCHYQRLPPHILWATGL
K
Human APOBEC-2:
(SEQ ID NO: 94)
MAQKEEAAVATEAASQNGEDLENLDDPEKLKELIELPPFEIVTGERLPANFFK
FQFRNVEYSSGRNKTFLCYVVEAQGKGGQVQASRGYLEDEHAAAHAEEAFFNTILP
AFDPALRYNVTWYVSSSPCAACADRIIKTLSKTKNLRLLILVGRLFMWEEPEIQAALK
KLKEAGCKLRIMKPQDFEYVWQNFVEQEEGESKAFQPWEDIQENFLYYEEKLADIL
K
Mouse APOBEC-2:
(SEQ ID NO: 95)
MAQKEEAAEAAAPASQNGDDLENLEDPEKLKELIDLPPFEIVTGVRLPVNFFK
FQFRNVEYSSGRNKTFLCYVVEVQSKGGQAQATQGYLEDEHAGAHAEEAFFNTILP
AFDPALKYNVTWYVSSSPCAACADRILKTLSKTKNLRLLILVSRLFMWEEPEVQAAL
KKLKEAGCKLRIMKPQDFEYIWQNFVEQEEGESKAFEPWEDIQENFLYYEEKLADIL
K
Rat APOBEC-2:
(SEQ ID NO: 96)
MAQKEEAAEAAAPASQNGDDLENLEDPEKLKELIDLPPFEIVTGVRLPVNFFK
FQFRNVEYSSGRNKTFLCYVVEAQSKGGQVQATQGYLEDEHAGAHAEEAFFNTILP
AFDPALKYNVTWYVSSSPCAACADRILKTLSKTKNLRLLILVSRLFMWEEPEVQAAL
KKLKEAGCKLRIMKPQDFEYLWQNFVEQEEGESKAFEPWEDIQENFLYYEEKLADIL
K
Bovine APOBEC-2:
(SEQ ID NO: 97)
MAQKEEAAAAAEPASQNGEEVENLEDPEKLKELIELPPFEIVTGERLPAHYFK
FQFRNVEYSSGRNKTFLCYVVEAQSKGGQVQASRGYLEDEHATNHAEEAFFNSIMP
TFDPALRYMVTWYVSSSPCAACADRIVKTLNKTKNLRLLILVGRLFMWEEPEIQAAL
RKLKEAGCRLRIMKPQDFEYIWQNFVEQEEGESKAFEPWEDIQENFLYYEEKLADIL
K
Petromyzon marinus CDA1 (pmCDA1)
(SEQ ID NO: 98)
MTDAEYVRIHEKLDIYTFKKQFFNNKKSVSHRCYVLFELKRRGERRACFWGYAVNK
PQSGTERGIHAEIFSIRKVEEYLRDNPGQFTINWYSSWSPCADCAEKILEWYNQELRG
NGHTLKIWACKLYYEKNARNQIGLWNLRDNGVGLNVMVSEHYQCCRKIFIQSSHNQ
LNENRWLEKTLKRAEKRRSELSIMIQVKILHTTKSPAV
Human APOBEC3G D316R_D317R
(SEQ ID NO: 99)
MKPHFRNTVERMYRDTFSYNFYNRPILSRRNTVWLCYEVKTKGPSRPPLDAKIFRGQ
VYSELKYHPEMRFFHWFSKWRKLHRDQEYEVTWYISWSPCTKCTRDMATFLAEDP
KVTLTIFVARLYYFWDPDYQEALRSLCQKRDGPRATMKIMNYDEFQHCWSKFVYS
QRELFEPWNNLPKYYILLHIMLGEILRHSMDPPTFTFNENNEPWVRGRHETYLCYEV
ERMHNDTWVLLNQRRGFLCNQAPHKHGFLEGRHAELCFLDVIPFWKLDLDQDYRV
TCFTSWSPCFSCAQEMAKFISKNKHVSLCIFTARIYRRQGRCQEGLRTLAEAGAKISI
MTYSEFKHCWDTFVDHQGCPFQPWDGLDEHSQDLSGRLRAILQNQEN
Human APOBEC3G chain A
(SEQ ID NO: 100)
MDPPTFTFNFNNEPWVRGRHETYLCYEVERMHNDTWVLLNQRRGFLCNQAPHKHG
FLEGRHAELCFLDVIPFWKLDLDQDYRVTCFTSWSPCFSCAQEMAKFISKNKHVSLCI
FTARIYDDQGRCQEGLRTLAEAGAKISIMTYSEFKHCWDTFVDHQGCPFQPWDGLD
EHSQDLSGRLRAILQ
Human APOBEC3G chain A D120R_D121R
(SEQ ID NO: 101)
MDPPTFTFNFNNEPWVRGRHETYLCYEVERMHNDTWVLLNQRRGFLCNQAP
HKHGFLEGRHAELCFLDVIPFWKLDLDQDYRVTCFTSWSPCFSCAQEMAKFISKNKH
VSLCIFTARIYRRQGRCQEGLRTLAEAGAKISIMTYSEFKHCWDTFVDHQGCPFQPW
DGLDEHSQDLSGRLRAILQ

Deaminase Domains that Modulate the Editing Window of Base Editors

Some aspects of the disclosure are based on the recognition that modulating the deaminase domain catalytic activity of any of the fusion proteins provided herein, for example by making point mutations in the deaminase domain, affect the processivity of the fusion proteins (e.g., base editors). For example, mutations that reduce, but do not eliminate, the catalytic activity of a deaminase domain within a base editing fusion protein can make it less likely that the deaminase domain will catalyze the deamination of a residue adjacent to a target residue, thereby narrowing the deamination window. The ability to narrow the deaminataion window may prevent unwanted deamination of residues adjacent of specific target residues, which may decrease or prevent off-target effects.

In some embodiments, any of the fusion proteins provided herein comprise a deaminase domain (e.g., a cytidine deaminase domain) that has reduced catalytic deaminase activity. In some embodiments, any of the fusion proteins provided herein comprise a deaminase domain (e.g., a cytidine deaminase domain) that has a reduced catalytic deaminase activity as compared to an appropriate control. For example, the appropriate control may be the deaminase activity of the deaminase prior to introducing one or more mutations into the deaminase. In other embodiments, the appropriate control may be a wild-type deaminase. In some embodiments, the appropriate control is a wild-type apolipoprotein B mRNA-editing complex (APOBEC) family deaminase. In some embodiments, the appropriate control is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, or an APOBEC3H deaminase. In some embodiments, the appropriate control is an activation induced deaminase (AID). In some embodiments, the appropriate control is a cytidine deaminase 1 from Petromyzon marinus (pmCDA1). In some embodiments, the deaminase domain may be a deaminase domain that has at least 1%, at least 5%, at least 15%, at least 20%, at least 25%, at least 30%, at least 40%, at least 50%, at least 60%, at least 70%, at least 80%, at least 90%, or at least 95% less catalytic deaminase activity as compared to an appropriate control.

In some embodiments, any of the fusion proteins provided herein comprise an APOBEC deaminase comprising one or more mutations selected from the group consisting of H121X, H122X, R126X, R126X, R118X, W90X, W90X, and R132X of rAPOBEC1 (SEQ ID NO: 93), or one or more corresponding mutations in another APOBEC deaminase, wherein X is any amino acid. In some embodiments, any of the fusion proteins provided herein comprise an APOBEC deaminase comprising one or more mutations selected from the group consisting of H121R, H122R, R126A, R126E, R118A, W90A, W90Y, and R132E of rAPOBEC1 (SEQ ID NO: 93), or one or more corresponding mutations in another APOBEC deaminase.

In some embodiments, any of the fusion proteins provided herein comprise an APOBEC deaminase comprising one or more mutations selected from the group consisting of D316X, D317X, R320X, R320X, R313X, W285X, W285X, R326X of hAPOBEC3G (SEQ ID NO: 77), or one or more corresponding mutations in another APOBEC deaminase, wherein X is any amino acid. In some embodiments, any of the fusion proteins provided herein comprise an APOBEC deaminase comprising one or more mutations selected from the group consisting of D316R, D317R, R320A, R320E, R313A, W285A, W285Y, R326E of hAPOBEC3G (SEQ ID NO: 77), or one or more corresponding mutations in another APOBEC deaminase.

In some embodiments, any of the fusion proteins provided herein comprise an APOBEC deaminase comprising a H121R and a H122R mutation of rAPOBEC1 (SEQ ID NO: 93), or one or more corresponding mutations in another APOBEC deaminase. In some embodiments, any of the fusion proteins provided herein comprise an APOBEC deaminase comprising a R126A mutation of rAPOBEC1 (SEQ ID NO: 93), or one or more corresponding mutations in another APOBEC deaminase. In some embodiments, any of the fusion proteins provided herein comprise an APOBEC deaminase comprising a R126E mutation of rAPOBEC1 (SEQ ID NO: 93), or one or more corresponding mutations in another APOBEC deaminase. In some embodiments, any of the fusion proteins provided herein comprise an APOBEC deaminase comprising a R118A mutation of rAPOBEC1 (SEQ ID NO: 93), or one or more corresponding mutations in another APOBEC deaminase. In some embodiments, any of the fusion proteins provided herein comprise an APOBEC deaminase comprising a W90A mutation of rAPOBEC1 (SEQ ID NO: 93), or one or more corresponding mutations in another APOBEC deaminase. In some embodiments, any of the fusion proteins provided herein comprise an APOBEC deaminase comprising a W90Y mutation of rAPOBEC1 (SEQ ID NO: 93), or one or more corresponding mutations in another APOBEC deaminase. In some embodiments, any of the fusion proteins provided herein comprise an APOBEC deaminase comprising a R132E mutation of rAPOBEC1 (SEQ ID NO: 93), or one or more corresponding mutations in another APOBEC deaminase. In some embodiments, any of the fusion proteins provided herein comprise an APOBEC deaminase comprising a W90Y and a R126E mutation of rAPOBEC1 (SEQ ID NO: 93), or one or more corresponding mutations in another APOBEC deaminase. In some embodiments, any of the fusion proteins provided herein comprise an APOBEC deaminase comprising a R126E and a R132E mutation of rAPOBEC1 (SEQ ID NO: 93), or one or more corresponding mutations in another APOBEC deaminase. In some embodiments, any of the fusion proteins provided herein comprise an APOBEC deaminase comprising a W90Y and a R132E mutation of rAPOBEC1 (SEQ ID NO: 93), or one or more corresponding mutations in another APOBEC deaminase. In some embodiments, any of the fusion proteins provided herein comprise an APOBEC deaminase comprising a W90Y, R126E, and R132E mutation of rAPOBEC1 (SEQ ID NO: 93), or one or more corresponding mutations in another APOBEC deaminase.

In some embodiments, any of the fusion proteins provided herein comprise an APOBEC deaminase comprising a D316R and a D317R mutation of hAPOBEC3G (SEQ ID NO: 77), or one or more corresponding mutations in another APOBEC deaminase. In some embodiments, any of the fusion proteins provided herein comprise an APOBEC deaminase comprising a R320A mutation of hAPOBEC3G (SEQ ID NO: 77), or one or more corresponding mutations in another APOBEC deaminase. In some embodiments, any of the fusion proteins provided herein comprise an APOBEC deaminase comprising a R320E mutation of hAPOBEC3G (SEQ ID NO: 77), or one or more corresponding mutations in another APOBEC deaminase. In some embodiments, any of the fusion proteins provided herein comprise an APOBEC deaminase comprising a R313A mutation of hAPOBEC3G (SEQ ID NO: 77), or one or more corresponding mutations in another APOBEC deaminase.

In some embodiments, any of the fusion proteins provided herein comprise an APOBEC deaminase comprising a W285A mutation of hAPOBEC3G (SEQ ID NO: 77), or one or more corresponding mutations in another APOBEC deaminase. In some embodiments, any of the fusion proteins provided herein comprise an APOBEC deaminase comprising a W285Y mutation of hAPOBEC3G (SEQ ID NO: 77), or one or more corresponding mutations in another APOBEC deaminase. In some embodiments, any of the fusion proteins provided herein comprise an APOBEC deaminase comprising a R326E mutation of hAPOBEC3G (SEQ ID NO: 77), or one or more corresponding mutations in another APOBEC deaminase. In some embodiments, any of the fusion proteins provided herein comprise an APOBEC deaminase comprising a W285Y and a R320E mutation of hAPOBEC3G (SEQ ID NO: 77), or one or more corresponding mutations in another APOBEC deaminase. In some embodiments, any of the fusion proteins provided herein comprise an APOBEC deaminase comprising a R320E and a R326E mutation of hAPOBEC3G (SEQ ID NO: 77), or one or more corresponding mutations in another APOBEC deaminase. In some embodiments, any of the fusion proteins provided herein comprise an APOBEC deaminase comprising a W285Y and a R326E mutation of hAPOBEC3G (SEQ ID NO: 77), or one or more corresponding mutations in another APOBEC deaminase. In some embodiments, any of the fusion proteins provided herein comprise an APOBEC deaminase comprising a W285Y, R320E, and R326E mutation of hAPOBEC3G (SEQ ID NO: 77), or one or more corresponding mutations in another APOBEC deaminase.

Fusion Proteins Comprising a Nuclease Programmable DNA Binding Protein (napDNAbp), a Cytidine Deaminase, and Multiple Uracil Binding Protein (UBP) Domains

Some aspects of the disclosure provide fusion proteins comprising a nucleic acid programmable DNA binding protein (napDNAbp), a cytidine deaminase, and a first and second uracil binding protein (UBP) domain. In some embodiments, any of the fusion proteins provided herein are base editors. In some embodiments, the UBP is a uracil modifying enzyme. In some embodiments, the UBP is a uracil base excision enzyme. In some embodiments, the UBP is a uracil DNA glycosylase. In some embodiments, the UBP is any of the uracil binding proteins provided herein. For example, the UBP may be a UDG, a UdgX, a UdgX*, a UdgX_On, or a SMUG1. In particular embodiments, the UBP domain is a UdgX. In some embodiments, the UBP comprises an amino acid sequence that is at least 75%, 80%, 85%, 90%, 95%, 96%, 97%, 98%, 99%, or 99.5% identical to a uracil binding protein, a uracil base excision enzyme or a uracil DNA glycosylase (UDG) enzyme. In some embodiments, the UBP comprises an amino acid sequence that is at least 75%, 80%, 85%, 90%, 95%, 96%, 97%, 98%, 99%, or 99.5% identical to any of the uracil binding proteins provided herein. For example, the UBP may comprise an amino acid sequence that is at least 75%, 80%, 85%, 90%, 95%, 96%, 97%, 98%, 99%, or 99.5% identical to any one of SEQ ID NOs: 48-53. In some embodiments, the UBP comprises the amino acid sequence of any one of SEQ ID NOs: 48-53.

In some embodiments, the napDNAbp is a Cas9 domain, a Cpf1 domain, a CasX domain, a CasY domain, a C2c1 domain, a C2c2 domain, aC2c3 domain, or an Argonaute domain. In some embodiments, the napDNAbp is any napDNAbp provided herein. In some embodiments, the napDNAbp is a Cas9 nickase, such as an nCas9-NG or a HF-nCas9 (or HF-nCas9-NG). The nCas9-NG variant has a PAM that corresponds to NGN. In some embodiments, the napDNAbp of any of the fusion proteins provided herein is a Cas9 domain. The Cas9 domain may be any of the Cas9 domains or Cas9 proteins (e.g., nCas9) provided herein. In some embodiments, any of the Cas9 domains or Cas9 proteins (e.g., nCas9) provided herein may be fused with any of the cytidine deaminases provided herein.

In some embodiments, the fusion protein wherein the fusion protein comprises the structure [cytidine deaminase domain]-[first UBP domain]-[napDNAbp domain], wherein each instance of “]-[” comprises an optional linker. The cytidine deaminase and the first UBP domain, and/or the first UBP domain and the napDNAbp domain, may be fused via a linker, such as a linker comprising the amino acid sequence of any one of SEQ ID NOs: 102-109 and 441. In particular embodiments, the fusion protein comprises the structure [cytidine deaminase domain]-[UdgX protein]-[Cas9 nickase], wherein each instance of “]-[” comprises an optional linker. In some embodiments, the fusion protein comprises the “AXC” architecture.

In some embodiments of the disclosed base editing fusion proteins, the second UBP domain and the cytidine deaminase domain are fused via a linker comprising the amino acid sequence of any one of SEQ ID NOs: 102-109 and 441. In some embodiments, the DNA repair protein and the cytidine deaminase domain are fused via a linker comprising the amino acid sequence of any one of SEQ ID NOs: 102-109 and 441. In some embodiments, the napDNAbp domain and the DNA repair protein are fused via a linker comprising the amino acid sequence of any one of SEQ ID NOs: 102-109 and 441. In some embodiments, the napDNAbp domain and the second DNA repair protein are fused via a linker comprising the amino acid sequence of any one of SEQ ID NOs: 102-109 and 441.

In some embodiments, any of the disclosed fusion proteins comprise the structure:
NH₂-[second UBP domain]-[cytidine deaminase domain]-[first UBP domain]-[napDNAbp domain]-COOH; or
NH₂-[second UBP domain]-[cytidine deaminase domain]-[first UBP domain]-[napDNAbp domain]-[third UBP domain]-COOH.

In some embodiments, the fusion proteins comprising a cytidine deaminase, a napDNAbp (e.g., Cas9 domain), and first and second UBP domains do not include a linker sequence. In some embodiments, a linker is present between the cytidine deaminase domain and the napDNAbp. In some embodiments, a linker is present between the cytidine deaminase domain and the UBP domains. In some embodiments, a linker is present between the napDNAbp and the UBP domains. In some embodiments, the “]-[” used in the general architecture above indicates the presence of an optional linker. In some embodiments, the cytidine deaminase and the napDNAbp, the cytidine deaminase and the first and/or second UBP domain, and/or the napDNAbp and the first and/or second UBP domain are fused via any of the linkers provided herein. For example, in some embodiments the cytidine deaminase and the napDNAbp, the cytidine deaminase and the first and/or second UBP domain, and/or the napDNAbp and the first and/or second UBP domain are fused via any of the linkers provided below in the section entitled “Linkers”. In some embodiments, the cytidine deaminase and the napDNAbp, the cytidine deaminase and the first and/or second UBP domain, and/or the napDNAbp and the first and/or second UBP domain are fused via a linker that comprises between 1 and 200 amino acids. In some embodiments, the cytidine deaminase and the napDNAbp, the cytidine deaminase and the first and/or second UBP domain, and/or the napDNAbp and the first and/or second UBP domain are fused via a linker that comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. In some embodiments, the cytidine deaminase and the napDNAbp, the cytidine deaminase and the first and/or second UBP domain, and/or the napDNAbp and the first and/or second UBP domain are fused via a linker that comprises 4, 16, 24, 32, 60, 91 or 104 amino acids in length. In some embodiments, the cytidine deaminase and the napDNAbp, the cytidine deaminase and the first and/or second UBP domain, and/or the napDNAbp and the first and/or second UBP domain are fused via a linker that comprises the amino acid sequence of SGSETPGTSESATPES (SEQ ID NO: 102), SGGS (SEQ ID NO: 103), SGGSSGSETPGTSESATPESSGGS (SEQ ID NO: 107), SGGSSGGSSGSETPGTSESATPESSGGSSGGS (SEQ ID NO: 108), SGGSSGGSSGSETPGTSESATPESAGSYPYDVPDYAGSAAPAAKKKKLDGSGSGGSS GGS (SEQ ID NO: 441), GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 109), or SGGSGGSGGS (SEQ ID NO: 120). In some embodiments, the cytidine deaminase and the napDNAbp, the cytidine deaminase and the first and/or second UBP domain, and/or the napDNAbp and the first and/or second UBP domain are fused via a linker comprising the amino acid sequence SGSETPGTSESATPES (SEQ ID NO: 102), which may also be referred to as the XTEN linker.

Fusion Proteins Comprising a Nuclease Programmable DNA Binding Protein (napDNAbp), a Cytidine Deaminase, a First Uracil Binding Protein Domain and a DNA Repair Protein

Some aspects of the disclosure provide fusion proteins comprising a nucleic acid programmable DNA binding protein (napDNAbp), a cytidine deaminase, a first UBP domain and a DNA repair protein. The DNA repair protein may be selected from a DNA polymerase, an exonuclease, an RNA binding motif protein, an E3 ligase, and a translesion polymerase. In particular embodiments, the DNA repair protein is one of POLD2, RBMX, and EXO1. In some embodiments, the DNA repair protein is a nucleic acid polymerase, such as a DNA polymerase (e.g., a translesion polymerase). In various embodiments, the DNA repair protein is selected from DNA polymerase D1 (POLD1), DNA polymerase D2 (POLD2), and DNA polymerase D3 (POLD3).

In some embodiments, the napDNAbp is a Cas9 nickase. In some embodiments, the napDNAbp is any napDNAbp provided herein. In some embodiments, the napDNAbp of any of the fusion proteins provided herein is a Cas9 domain. The Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases provided herein.

In some embodiments, any of the disclosed fusion proteins comprise the structure:
NH₂-[second UBP domain]-[cytidine deaminase domain]-[first UBP domain]-[napDNAbp domain]-[DNA repair protein]-COOH;
NH₂-[DNA repair protein]-[cytidine deaminase domain]-[first UBP domain]-[napDNAbp domain]-COOH;
NH₂-[DNA repair protein]-[cytidine deaminase domain]-[first UBP domain]-[napDNAbp domain]-[second UBP domain]-COOH; and
NH₂-[DNA repair protein]-[cytidine deaminase domain]-[first UBP domain]-[napDNAbp domain]-[second DNA repair protein]-COOH; or
NH₂-[DNA repair protein]-[cytidine deaminase domain]-[first UBP domain]-[napDNAbp domain]-[second DNA repair protein]-[second UBP domain]-COOH.

In some embodiments, the fusion proteins comprising a cytidine deaminase, a napDNAbp (e.g., Cas9 domain) domain, first UBP domain, and DNA repair protein do not include a linker sequence. In some embodiments, a linker is present between the cytidine deaminase domain and the napDNAbp domain. In some embodiments, a linker is present between the cytidine deaminase domain and the first UBP domain. In some embodiments, a linker is present between the cytidine deaminase domain, or the napDNAbp domain, and the DNA repair protein. In some embodiments, a linker is present between the napDNAbp domain and the first UBP domain. In some embodiments, the “]-[” used in the general architecture above indicates the presence of an optional linker. In some embodiments, the cytidine deaminase and the napDNAbp domain, the cytidine deaminase and the first UBP domain, the cytidine deaminase domain and the DNA repair protein, and/or the napDNAbp domain and the DNA repair protein are fused via any of the linkers provided herein, such as any of the linkers provided below in the section entitled “Linkers”. In some embodiments, cytidine deaminase and the napDNAbp domain, the cytidine deaminase and the first UBP domain, the cytidine deaminase domain and the DNA repair protein, and/or the napDNAbp domain and the DNA repair protein are fused via a linker that comprises between 1 and 200 amino acids. In some embodiments, the cytidine deaminase and the napDNAbp domain, the cytidine deaminase and the first UBP domain, the cytidine deaminase domain and the DNA repair protein, and/or the napDNAbp domain and the DNA repair protein are fused via a linker that comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60, 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. In some embodiments, cytidine deaminase and the napDNAbp domain, the cytidine deaminase and the first UBP domain, the cytidine deaminase domain and the DNA repair protein, and/or the napDNAbp domain and the DNA repair protein are fused via a linker that comprises 4, 16, 24, 32, 60, 91, or 104 amino acids in length. In some embodiments, the cytidine deaminase and the napDNAbp domain, the cytidine deaminase and the first UBP domain, the cytidine deaminase domain and the DNA repair protein, and/or the napDNAbp domain and the DNA repair protein are fused via a linker that comprises the amino acid sequence of SGSETPGTSESATPES (SEQ ID NO: 102), SGGS (SEQ ID NO: 103), SGGSSGSETPGTSESATPESSGGS (SEQ ID NO: 107), SGGSSGGSSGSETPGTSESATPESSGGSSGGS (SEQ ID NO: 108), SGGSSGGSSGSETPGTSESATPESAGSYPYDVPDYAGSAAPAAKKKKLDGSGSGGSS GGS (SEQ ID NO: 441), GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 109), or SGGSGGSGGS (SEQ ID NO: 120). In some embodiments, cytidine deaminase and the napDNAbp domain, the cytidine deaminase and the first UBP domain, the cytidine deaminase domain and the DNA repair protein, and/or the napDNAbp domain and the DNA repair protein are fused via a linker comprising the amino acid sequence SGSETPGTSESATPES (SEQ ID NO: 102), which may also be referred to as the XTEN linker.

### Nuclear Localization Sequences (NLS)

In some embodiments, any of the fusion proteins provided herein further comprise one or more nuclear targeting sequences, for example, a nuclear localization sequence (NLS).

In some embodiments, a NLS comprises an amino acid sequence that facilitates the importation of a protein, that comprises an NLS, into the cell nucleus (e.g., by nuclear transport). In some embodiments, the NLS is a bipartite NLS (BPNLS). Bipartite NLSs are separated by a relatively short spacer sequence (e.g., from 2-20 amino acids, from 5-15 amino acids, or from 8-12 amino acids).

In some embodiments, any of the fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the napDNAbp domain. In some embodiments, the NLS is fused to the C-terminus of the napDNAbp domain. In some embodiments, the NLS is fused to the N-terminus of the cytidine deaminase domain. In some embodiments, the NLS is fused to the C-terminus of the cytidine deaminase domain.

In some embodiments, the NLS is fused to the N-terminus of the first UBP domain or the second UBP domain. In some embodiments, the NLS is fused to the C-terminus of the the first UBP domain or the second UBP domain. In some embodiments, the NLS is fused to the N-terminus of the DNA repair protein. In some embodiments, the NLS is fused to the C-terminus of the DNA repair protein. In some embodiments, the NLS is fused to the C-terminus of the second DNA repair protein.

In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. In some embodiments, the NLS comprises an amino acid sequence as set forth in SEQ ID NO: 41 or SEQ ID NO: 42. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, a NLS comprises the amino acid sequence

(SEQ ID NO: 41)
PKKKRKV,
(SEQ ID NO: 42)
MDSLLMNRRKFLYQFKNVRWAKGRRETYLC,
(SEQ ID NO: 43)
KRTADGSEFESPKKKRKV,
(SEQ ID NO: 44)
KRGINDRNFWRGENGRKTR,
(SEQ ID NO: 45)
KKTGGPIYRRVDGKWRR,
(SEQ ID NO: 46)
RRELILYDKEEIRRIWR,
(SEQ ID NO: 47)
AVSRKRKA,
or
(SEQ ID NO: 440)
KRTADGSEFEPKKKRKV.

Exemplary fusion proteins of the disclosure comprising one or more NLSs may comprise one of the following structures:
NH₂-[BPNLS]-[second UBP domain]-[cytidine deaminase domain]-[first UBP domain]-[napDNAbp domain]-[BPNLS]-COOH;
NH₂-[BPNLS]-[second UBP domain]-[cytidine deaminase domain]-[first UBP domain]-[napDNAbp domain]-[DNA repair protein]-[BPNLS]-COOH;
NH₂-[BPNLS]-[DNA repair protein]-[cytidine deaminase domain]-[first UBP domain]-[napDNAbp domain]-[BPNLS]-COOH;
NH₂-[BPNLS]-[second UBP domain]-[cytidine deaminase domain]-[first UBP domain]-[napDNAbp domain]-[third UBP domain]-[BPNLS]-COOH;
NH₂-[BPNLS]-[DNA repair protein]-[cytidine deaminase domain]-[first UBP domain]-[napDNAbp domain]-[second UBP domain]-[BPNLS]-COOH; and
NH₂-[BPNLS]-[DNA repair protein]-[cytidine deaminase domain]-[first UBP domain]-[napDNAbp domain]-[second DNA repair protein]-[BPNLS]-COOH;
   wherein each instance of “]-[” comprises an optional linker.

### Linkers

In certain embodiments, linkers may be used to link any of the proteins or protein domains described herein. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond (e.g., a covalent bond), an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. In some embodiments, a linker comprises the amino acid sequence SGSETPGTSESATPES (SEQ ID NO: 102), which may also be referred to as the XTEN linker. In some embodiments, a linker comprises the amino acid sequence SGGS (SEQ ID NO: 103). In some embodiments, a linker comprises (SGGS)ₙ (SEQ ID NO: 103), (GGGS)ₙ (SEQ ID NO: 104), (GGGGS)ₙ (SEQ ID NO: 105), (G)ₙ(SEQ ID NO: 121), (EAAAK)ₙ (SEQ ID NO: 106), (GGS)ₙ(SEQ ID NO: 122), SGSETPGTSESATPES (SEQ ID NO: 102), SGGSGGSGGS (SEQ ID NO: 120), or (XP)ₙ motif (SEQ ID NO: 123), or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15. In some embodiments, a linker comprises SGSETPGTSESATPES (SEQ ID NO: 102), and SGGS (SEQ ID NO: 103). In some embodiments, a linker comprises SGGSSGSETPGTSESATPESSGGS (SEQ ID NO: 107).

In some embodiments, a linker comprises SGGSSGGSSGSETPGTSESATPESSGGSSGGS (SEQ ID NO: 108). In some embodiments, the linker comprises SGGSSGGSSGSETPGTSESATPESAGSYPYDVPDYAGSAAPAAKKKKLDGSGSGGSS GGS (SEQ ID NO: 441). In some embodiments, a linker comprises GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 109). In some embodiments, a linker comprises SGGSGGSGGS (SEQ ID NO: 120).

In some embodiments, the linker is 32 amino acids in length (e.g., the linker consists of SEQ ID NO: 108). In some embodiments, the linker is 60 amino acids in length (e.g., the linker consists of SEQ ID NO: 441).

### Guide Nucleic Acids

Some aspects of this disclosure provide complexes comprising any of the fusion proteins provided herein, and a guide nucleic acid bound to napDNAbp of the fusion protein. Some aspects of this disclosure provide complexes comprising any of the fusion proteins provided herein, and a guide RNA bound to a Cas9 domain (e.g., a dCas9, a nuclease active Cas9, or a Cas9 nickase) of fusion protein.

In various embodiments, the present disclosure further provides guide RNAs for use in accordance with the disclosed methods of editing. The disclosure provides guide RNAs that are designed to recognize target sequences. Such gRNAs may be designed to have guide sequences (or “spacers”) having complementarity to a protospacer within the target sequence.

Guide RNAs are also provided for use with one or more of the disclosed fusion proteins, e.g., in the disclosed methods of editing a nucleic acid molecule. Such gRNAs may be designed to have guide sequences having complementarity to a protospacer within a target sequence to be edited, and to have backbone sequences that interact specifically with the napDNAbp domains of any of the disclosed fusion proteins, such as Cas9 nickase domains of the disclosed fusion proteins.

In various embodiments, the fusion proteins may be complexed, bound, or otherwise associated with (e.g., via any type of covalent or non-covalent bond) one or more guide sequences. The guide sequence becomes associated or bound to the base editor and directs its localization to a specific target sequence having complementarity to the guide sequence or a portion thereof. The particular design embodiments of a guide sequence will depend upon the nucleotide sequence of a genomic target sequence (i.e., the desired site to be edited) and the type of napDNAbp (e.g., type of Cas9 protein) present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of the napDNAbp (e.g., a Cas9 or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a target sequence (or off-target site).

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence. Similarly, cleavage of a target polynucleotide sequence may be evaluated in situ by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target Cny target sequence. In some embodiments, the target sequence is a sequence within a genome of a cell. Exemplary target sequences include those that are unique in the target genome.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and P A Carr & G M Church, 2009, Nature Biotechnology 27(12): 1151-62). Additional algorithms may be found in Chuai, G. et al., DeepCRISPR: optimized CRISPR guide RNA design by deep learning, Genome Biol. 19:80 (2018), and U.S. Application Ser. No. 61/836,080 and U.S. Pat. No. 8,871,445, issued Oct. 28, 2014, the entireties of each of which are incorporated herein by reference.

The guide sequence of the gRNA is linked to a tracr mate (also known as a “backbone”) sequence which in turn hybridizes to a tracr sequence. A tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In certain embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides.

Non-limiting examples of single (DNA) polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence (6 Ts) represents the transcriptional terminator:
(1) NNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 216);
(2) NNNNNNNNNNNNNNNNNNgtttttgtactctcaGAAAtgcagaagctacaaagataaggcttcatgccgaaatca acaccctgtcattttatggcagggtgttttcgttatttaaTTTTTT (SEQ ID NO: 217);
(3) NNNNNNNNNNNNNNNNNNNNgtttttgtactctcaGAAAtgcagaagctacaaagataaggcttcatgccgaa atcaacaccctgtcattttatggcagggtgtTTTTT (SEQ ID NO: 218);
(4) NNNNNNNNNNNNNNNNNNNNgttttagagctaGAAAtagcaagttaaaataaggctagtccgttatcaacttg aaaaagtggcaccgagtcggtgcTTTTTT (SEQ ID NO: 219);
(5) NNNNNNNNNNNNNNNNNNNgttttagagctaGAAATAGcaagttaaaataaggctagtccgttatcaacttga aaaagtgTTTTTTT (SEQ ID NO: 220); and
(6) NNNNNNNNNNNNNNNNNNNNgttttagagctagAAATAGcaagttaaaataaggctagtccgttatcaTT TTTTTT (SEQ ID NO: 221). In some embodiments, sequences (1) to (3) are used in combination with Cas9 from S. Thermophiles CRISPR1. In some embodiments, sequences (4) to (6) are used in combination with Cas9 from S. pyogenes. In some embodiments, the tracr sequence is a separate transcript from a transcript comprising the tracr mate sequence.

In some embodiments, the guide RNAs for use in accordance with the disclosed methods of editing comprise synthetic single guide RNAs (sgRNAs) containing modified ribonucleotides. In some embodiments, the guide RNAs contain modifications such as 2′-O-methylated nucleotides and phosphorothioate linkages. In some embodiments, the guide RNAs contain 2′-O-methyl modifications in the first three and last three nucleotides, and phosphorothioate bonds between the first three and last three nucleotides. Exemplary modified synthetic sgRNAs are disclosed in Hendel A. et al., Nat. Biotechnol. 33, 985-989 (2015), herein incorporated by reference.

In some embodiments, the guide RNAs for use in accordance with the disclosed methods of editing comprise a backbone structure that is recognized by an S. pyogenes Cas9 protein or domain, such as an SpCas9 domain of the disclosed fusion proteins. The backbone structure recognized by an SpCas9 protein may comprise the sequence 5′-[guide sequence]-guuuuagagcuagaaauagcaaguuaaaauaaggcuaguccguuaucaacuugaaaaaguggcaccgagucggugcuuu uu-3′ (SEQ ID NO: 119), wherein the guide sequence comprises a sequence that is complementary to the protospacer of the target sequence. See U.S. Publication No. 2015/0166981, published Jun. 18, 2015, the disclosure of which is incorporated by reference herein. The guide sequence is typically 20 nucleotides long.

In other embodiments, the guide RNAs for use in accordance with the disclosed methods of editing comprise a backbone structure that is recognized by an S. aureus Cas9 protein. The backbone structure recognized by an SaCas9 protein may comprise the sequence 5′-[guide sequence]-guuuuaguacucuguaaugaaaauuacagaaucuacuaaaacaaggcaaaaugccguguuuaucucgucaacuuguugg cgagauuuuuuu-3′ (SEQ ID NO: 222).

In other embodiments, the guide RNAs for use in accordance with the disclosed methods of editing comprise a backbone structure that is recognized by an Lachnospiraceae bacterium Cas12a protein. The backbone structure recognized by an LbCas12a protein may comprise the sequence 5′-[guide sequence]-uaauuucuacuaaguguagau-3′ (SEQ ID NO: 445).

In other embodiments, the guide RNAs for use in accordance with the disclosed methods of editing comprise a backbone structure that is recognized by an Acidaminococcus sp. BV3L6 Cas12a protein. The backbone structure recognized by an AsCas12a protein may comprise the sequence 5′-[guide sequence]-uaauuucuacucuuguagau-3′ (SEQ ID NO: 446).

The sequences of suitable guide RNAs for targeting the disclosed ABEs to specific genomic target sites will be apparent to those of skill in the art based on the present disclosure. Such suitable guide RNA sequences typically comprise guide sequences that are complementary to a nucleic sequence within 50 nucleotides upstream or downstream of the target nucleobase pair to be edited. Some exemplary guide RNA sequences suitable for targeting any of the provided ABEs to specific target sequences are provided herein. Additional guide sequences are are well known in the art and may be used with the fusion proteins described herein. Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are incorporated herein by reference.

In some embodiments, the 3′ end of the target sequence is immediately adjacent to a canonical PAM sequence (NGG). In some embodiments, the guide nucleic acid (e.g., guide RNA) is complementary to a sequence associated with a disease or disorder. In some embodiments, the guide nucleic acid (e.g., guide RNA) is complementary to a sequence associated with a disease or disorder having a mutation in a gene associated with any of the diseases or disorders provided herein. In some embodiments, the guide nucleic acid (e.g., guide RNA) is complementary to any of the genes associated with a disease or disorder as provided herein.

### Vectors

Several aspects of the making and using the fusion proteins of the disclosure relate to vector systems comprising one or more vectors encoding the fusion proteins. Vectors may be designed to clone and/or express the fusion proteins of the disclosure. Vectors may also be designed to transfect the fusion proteins of the disclosure into one or more cells, e.g., a target diseased eukaryotic cell for treatment with the base editor systems and methods disclosed herein.

Vectors may be designed for expression of base editor transcripts (e.g. nucleic acid transcripts, proteins, or enzymes) in prokaryotic or eukaryotic cells. For example, base editor transcripts may be expressed in bacterial cells such as Escherichia coli, insect cells (using baculovirus expression vectors), yeast cells, plant cells, or mammalian cells. Suitable host cells are discussed further in Goeddel, Gene Expression Technology: Methods In Enzymology 185, Academic Press. San Diego, Calif. (1990). Alternatively, expression vectors encoding one or more fusion proteins described herein may be transcribed and translated in vitro, for example using T7 promoter regulatory sequences and T7 polymerase. Vectors encoding the fusion proteins provided herein may comprise any of the DNA plasmids identified at the Addgene webpage. Exemplary vectors include vectors encoding the the POLD2-rAPOBEC1-UdgX-nCas9-UdgX; UdgX-EE-UdgX-nCas9-UdgX, and UdgX-Anc689-UdgX-nCas9-RBMX base editing fusion proteins.

Vectors may be introduced and propagated in a prokaryotic cells. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins.

Fusion expression vectors also may be used to express the fusion proteins of the disclosure. Such vectors generally add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the base editor. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector drives protein expression in insect cells using baculovirus expression vectors. Baculovirus vectors available for expression of proteins in cultured insect cells (e.g., Sf9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (Campes and Tilghman, 1989. Genes Dev. 3: 537-546).

Eukaryotic Cell Systems for Determining Off-Target Effects of Fusion proteins

In some aspects, eukaryotic cell assays and systems for measuring off-target effects (e.g., off-target editing frequencies) of an fusion protein are provided. These systems may be used in accordance with the disclosed methods. These systems are referred to in the Examples as an “orthogonal R-loop assay.” Systems for determining the off-target editing frequency of a base editor may comprise one or more eukaryotic cells each comprising i) a first nucleic acid molecule encoding a base editor comprising a napDNAbp domain; (ii) a second nucleic acid molecule encoding a first guide RNA that is engineered to bind to the napDNAbp domain of the base editor, wherein the first guide RNA comprises a first sequence of at least 10 contiguous nucleotides that is complementary to a target sequence; (iii) a third nucleic acid molecule encoding a nuclease inactive napDNAbp protein; and (iv) a fourth nucleic acid molecule encoding a second gRNA that is engineered to bind to the nuclease inactive napDNAbp protein, wherein the second guide RNA comprises a second sequence of at least 10 contiguous nucleotides that is complementary to a third sequence, whereby the first complex and second complex generate two or more R-loops, and wherein the third sequence has about 60% or less sequence identity to the target sequence. Exemplary eukaryotic cell assays and systems for measuring off-target effects of the disclosed fusion proteins are disclosed in and International Application No. PCT/US2020/624628, filed Nov. 25, 2020, incorporate herein by reference.

The disclosed systems may further comprise a third, fourth, fifth, and/or sixth complex, wherein each of the third, fourth, fifth, and/or sixth complexes comprises (v) a second nuclease inactive napDNAbp protein, and (vi) a third guide RNA that is engineered to bind to the second nuclease inactive napDNAbp protein, wherein the third guide RNA comprises a fourth sequence of at least 10 contiguous nucleotides that is complementary to the third sequence. These complexes may be identical or essentially identical to each other, in that they are associated with identical or nearly identical gRNAs that have complementarity to the same off-target sequence. Any one of these complexes may be distinct or essentially identical to the second complex. The second and third guide RNA may share at least 95%, 98%, 98.5%, or 100% sequence identity, e.g., in the backbone of the guide RNA sequence. In certain embodiments, the second and third guide RNA share 100% identity or are the same. Likewise, the first nuclease inactive napDNAbp protein and the second nuclease inactive napDNAbp may be the same.

In some embodiments, any of the the nuclease inactive napDNAbp proteins of the described systems may be a dead Cas9 (dCas9) protein. Accordingly, in some embodiments, the second complex comprises a first dCas9 protein, and the third and subsequent complexes comprise a second dCas9 protein. In some embodiments, the nuclease inactive napDNAbp protein of any of the described complexes is a dead Cas9 protein from S. aureus. In some embodiments, the nuclease inactive napDNAbp protein is a dead Cas9 protein from S. pyogenes.

In some embodiments, the eukaryotic cells of the disclosed systems comprise mammalian cells. The eukaryotic cells may comprise human cells, e.g. HEK293T cells.

In some embodiments of these methods, transformed eukaryotic cells are sequenced to validate that mutations arise from cytosine-to-guanine conversions. This sequencing step may be achieved by Sanger sequencing, high-throughput sequencing, whole genome sequencing, and/or other sequencing methods known in the art.

### Methods of Using Fusion Proteins

Some aspects of this disclosure provide methods of using any of the fusion proteins (e.g., fusion proteins) provided herein, or complexes comprising a guide nucleic acid (e.g., gRNA) and a fusion protein (e.g., base editor) provided herein. For example, some aspects of this disclosure provide methods comprising contacting a DNA, or RNA molecule with any of the fusion proteins or fusion proteins provided herein, and with at least one guide nucleic acid (e.g., guide RNA), wherein the guide nucleic acid, (e.g., guide RNA) is about 15-100 nucleotides long and comprises a sequence of at least 10 contiguous nucleotides that is complementary to a target sequence. In some embodiments, the 3′ end of the target sequence is immediately adjacent to a canonical spCas9 PAM sequence (NGG). In some embodiments, the 3′ end of the target sequence is not immediately adjacent to a spCas9 canonical PAM sequence (NGG). In some embodiments, the 3′ end of the target sequence is immediately adjacent to an AGC, GAG, TTT, GTG, or CAA sequence.

In some embodiments, the target DNA sequence comprises a sequence associated with a disease or disorder. In some embodiments, the target DNA sequence comprises a point mutation associated with a disease or disorder. In some embodiments, the activity of the fusion protein (e.g., comprising a napDNAbp, a cytidine deaminase, and a uracil binding protein UBP), or the complex, results in a correction of the point mutation. In some embodiments, the target DNA sequence comprises a G to C, or C to G point mutation associated with a disease or disorder, and wherein deamination a mutant C base and excision of the resulting uracil results in a sequence that is not associated with a disease or disorder. In some embodiments, the target DNA sequence encodes a protein, and the point mutation is in a codon and results in a change in the amino acid encoded by the mutant codon as compared to the wild-type codon. In some embodiments, the deamination of the mutant C and excision of the resulting uracil results in a change of the amino acid encoded by the mutant codon. In some embodiments, the deamination of the mutant C and excision of the resulting uracil results in the codon encoding the wild-type amino acid. In some embodiments, the contacting is in vivo in a subject.

Some embodiments provide methods for using the DNA editing fusion proteins provided herein. In some embodiments, the fusion protein is used to introduce a point mutation into a nucleic acid by deaminating a target nucleobase, e.g., a C residue. In some embodiments, the fusion protein is used to deaminate a target C to U, which is then removed to create an abasic site previously occupied by the C residue. In some embodiments, the deamination of the target nucleobase, and a subsequent excision, results in the correction of a genetic defect, e.g., in the correction of a point mutation that leads to a loss of function in a gene product. In some embodiments, the methods provided herein are used to introduce a deactivating point mutation into a gene or allele that encodes a gene product that is associated with a disease or disorder. For example, in some embodiments, methods are provided herein that employ a DNA editing fusion protein to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provided herein is to restore the function of a dysfunctional gene via genome editing. The base editing fusion proteins provided herein can be validated for gene editing-based human therapeutics in vitro, e.g., by correcting a disease-associated mutation in human cell culture. It will be understood by the skilled artisan that the base editing fusion proteins provided herein, e.g., the fusion proteins comprising a nucleic acid programmable DNA binding protein (e.g., Cas9), a cytidine deaminase, and a uracil binding protein can be used to correct any single point C to G or G to C mutation. In the first case, deamination of the mutant C to U, and subsequent excision of the U, corrects the mutation, and in the latter case, deamination of the C to U, and subsequent excision of the U that is base-paired with the mutant G, followed by a round of replication, corrects the mutation.

The successful correction of point mutations in disease-associated genes and alleles opens up new strategies for gene correction with applications in therapeutics and basic research. Site-specific single-base modification systems like the disclosed fusion proteins comprising a nucleic acid programmable DNA binding protein (napDNAbp), a cytidine deaminase, and a uracil binding protein also have applications in “reverse” gene therapy, where certain gene functions are purposely suppressed or abolished. In these cases, site-specifically mutating residues that lead to inactivating mutations in a protein, or mutations that inhibit function of the protein can be used to abolish or inhibit protein function in vitro, ex vivo, or in vivo.

The instant disclosure provides methods for the treatment of a subject diagnosed with a disease associated with or caused by a point mutation that can be corrected by a DNA editing fusion protein provided herein. For example, in some embodiments, a method is provided that comprises administering to a subject having such a disease, e.g., a cancer associated with a point mutation as described above, an effective amount of a base editor fusion protein that corrects the point mutation (e.g., a C to G or G to C point mutation) or introduces a deactivating mutation into a disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

The instant disclosure provides lists of genes comprising pathogenic G to C or C to G mutations. Such pathogenic G to C or C to G mutations may be corrected using the methods and compositions provided herein, for example by mutating the C to a G, and/or the G to a C, thereby restoring gene function.

In some embodiments, a fusion protein recognizes canonical PAMs and therefore can correct the pathogenic G to C or C to G mutations with canonical PAMs, e.g., NGG, respectively, in the flanking sequences. For example, Cas9 proteins that recognize canonical PAMs comprise an amino acid sequence that is at least 80%, 85%, 90%, 95%, 97%, 98%, or 99% identical to the amino acid sequence of Streptococcus pyogenes Cas9 as provided by SEQ ID NO: 6, or to a fragment thereof comprising the RuvC and HNH domains of SEQ ID NO: 6.

Any of the fusion protein-gRNA complexes provided herein may be introduced into the cell for multiplexed base editing in any suitable way, either stably or transiently. In some embodiments, a base editor may be transfected into the cell. In some embodiments, the cell may be transduced or transfected with a nucleic acid construct that encodes the base editor. For example, a cell may be transduced (e.g. with a virus encoding a base editor) or transfected (e.g. with a plasmid encoding a base editor) with a nucleic acid that encodes the base editor. Alternatively, a cell may be introduced with the base editor itself. Such transduction may be a stable or transient transduction. In some embodiments, cells expressing a base editing base editor, or comprising a base editor, may be transduced or transfected with one or more gRNA molecules, for example, when the base editor comprises a Cas9 (e.g., nCas9) domain. In some embodiments, a plasmid expressing a base editor may be introduced into cells through electroporation (e.g., using an ATX MaxCyte electroporator), transient transfection (e.g. lipofection) or stable genome integration (e.g., piggybac), viral transduction, or other methods known to those of skill in the art.

In certain embodiments of the disclosed methods, the constructs that encode the fusion proteins are transfected into the cell separately from the constructs that encode the gRNAs. In certain embodiments, these components are encoded on a single construct and transfected together. In particular embodiments, these single constructs encoding the fusion proteins and gRNAs may be transfected into the cell iteratively, with each iteration associated with a subset of target sequences. In particular embodiments, these single constructs may be transfected into the cell over a period of days. In other embodiments, they may be transfected into the cell over a period of hours. In other embodiments, they may be transected into the cell over a period of weeks.

In the disclosed methods, target cells may be incubated with the base editor-gRNA complexes for two days, or 48 hours, after transfection to achieve multiplexed base editing. Target cells may be incubated for 30 hours, 40 hours, 54 hours, 60 hours, or 72 hours after transfection. Target cells may be incubated with the base editor-gRNA complexes for four days, five days, seven days, nine days, eleven days, or thirteen days or more after transfection.

In some aspects, the disclosure provides pharmaceutical compositions comprising a plurality of any of the fusion proteins described herein and a gRNA, wherein at least five of the fusion proteins of the plurality are each bound to a unique gRNA, and a pharmaceutically acceptable excipient.

In some aspects, the disclosure provides systematic and comprehensive predictive tools (e.g., one or more machine learning models, such as the BE-Hive model) that facilitate the selection of appropriate base editors to achieve any given desired predicted genotype outcome for a given target site through base editing. In another aspect, the predictive tools (e.g., machine learning models) disclosed herein may also be used to discover or identify previously unknown base editor properties (e.g., previously unknown preferences, such as a base editor's preference to make a transversion edit instead of a transition edit), which may facilitate the design of novel base editors with new capabilities. In various aspects, the disclosed machine learning models for selecting an appropriate base editor to achieve a desired genotype outcome may involve the consideration of one or more determinants of base editing, which can include, but are not limited to, the choice of the napDNAbp domain of the base editing system; the choice of the deaminase domain of the base editing system; the choice of the uracil binding protein(s) of the base editing system; the choice of the DNA repair protein of the base editing system; the choice of base editor; the target nucleotide sequence (e.g., guide RNA binding sites); the target genomic location; the transcriptional state of the target genomic location; locus-dependent activity of the choice napDNAbp; cell-type; transcriptional state of DNA repair proteins; and base editor modifications.

Accordingly, provided herein are methods of using at least one machine learning model to identify at least one fusion protein from among a set of fusion proteins, for use in a base editing system for introducing a desired cytosine-to-guanine edit into a nucleotide sequence, the at least one fusion protein comprising a napDNAbp domain, a cytidine deaminase domain, and at least one uracil binding protein, the method comprising: using software executing on at least one computer hardware processor to perform: obtaining input data indicative of the nucleotide sequence, one or more guide RNAs, and the set of fusion proteins; generating first input features from the input data; applying a first machine learning model to the first input features to obtain first output data indicative, for each fusion protein in the set, of a base editing efficiency at one or multiple locations in the nucleotide sequence, of the base editing system when using the each fusion protein; generating second input features from the input data; applying a second machine learning model to the second input features to obtain second output data indicative, for each fusion protein in the set, of a base editing product purity at one or multiple locations in the nucleotide sequence, by the base editing system when using the each fusion protein; and identifying, using the first output data and the second output data, at least one fusion protein for use in the base editing system for introducing the cytosine to guanine change in the nucleotide sequence. In some embodiments, the methods further comprise applying a third machine learning model to the second input features to obtain third output data indicative, for each fusion protein in the set, of a bystander editing efficiency at one or multiple locations in the nucleotide sequence, by the base editing system when using the each fusion protein.

In some embodiments, the set of fusion proteins comprises any of the fusion proteins disclosed herein. In some embodiments, the set of fusion proteins comprises any of the fusion proteins disclosed herein and any of the CGBEs disclosed in International Publication No. WO 2018/165629, published Sep. 13, 2018; Kurt, I. C. et al. Nature Biotechnology 39, 41-46 (2020); Zhao, D. et al. Nature Biotechnology 39, 35-40 (2020); and Chen, L. et al., Nature Communications 12 (2021), each of which are incorporated by reference herein. In some embodiments, the set of fusion proteins comprises mini CGBE1, CGBE1, APO1-nCas9-UNG, and APO1-nCas9-XRCC1.

Accordingly, provided herein are trained CGBE-Hive algorithms that accurately predict CGBE efficiency, C•G-to-G•C editing purity, and bystander editing patterns (R=0.90) to enable consistently pure CGBE editing that outperforms previously described CGBEs. Computational prediction of optimal CGBE-gRNA pairs enables high-purity C-to-G base editing at >4-fold more target sites than can be achieved using any single CGBE variant. Methods of Treatment

The present disclosure provides methods for the treatment of a subject diagnosed with a disease associated with or caused by a G:C to C:G point mutation that may be corrected by a DNA editing base editor provided herein. For example, in some embodiments, a method is provided that comprises administering to a subject having such a disease, e.g., a cancer associated with a point mutation as described above, an effective amount of an cytosine deaminase base editor that corrects the point mutation or introduces a deactivating mutation into a disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that may be treated by correcting a point mutation or introducing a deactivating mutation into a disease-associated gene will be known to those of skill in the art, and the disclosure is not limited in this respect.

In some embodiments, the deamination of the mutant C base and excision of the resulting uracil results in the codon encoding the wild-type amino acid. In some embodiments, the contacting is in vivo in a subject. In some embodiments, the subject has or has been diagnosed with a disease or disorder. In some embodiments, the disease or disorder is a hemoglobinopathy. In some embodiments, the disease or disorder is sickle cell disease. In some embodiments, the disease or disorder is Ehlers-Danlos syndrome, Sotos syndrome, Cornelia de Lange syndrome, Perlmen Syndrome, or a cancer.

Some embodiments provide methods for using the fusion proteins provided herein. In some embodiments, the fusion proteins are used to introduce a point mutation into a nucleic acid by deaminating a target nucleobase, e.g., a C residue. In some embodiments, the deamination of the target C base and excision of the resulting uracil results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a DNA editing base editor to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provided herein is to restore the function of a dysfunctional gene via genome editing. The nucleobase editing proteins provided herein can be validated for gene editing-based human therapeutics in vitro, e.g., by correcting a disease-associated mutation in human cell culture. It will be understood by the skilled artisan that the nucleobase editing proteins provided herein, e.g., the fusion proteins comprising a nucleic acid programmable DNA binding protein (e.g., Cas9) and an cytosine deaminase domain may be used to correct any single point C to G mutation.

The present disclosure provides methods for the treatment of additional diseases or disorders, e.g., diseases or disorders that are associated or caused by a G:C to C:G point mutation that may be corrected by any of the base editors or editing methods disclosed herein. Some such diseases are described herein, and additional suitable diseases that may be treated with the strategies and fusion proteins provided herein will be apparent to those of skill in the art based on the present disclosure. Exemplary suitable diseases and disorders are listed below. Exemplary suitable diseases and disorders include, without limitation: 2-methyl-3-hydroxybutyric aciduria; 3 beta-Hydroxysteroid dehydrogenase deficiency; 3-Methylglutaconic aciduria; 3-Oxo-5 alpha-steroid delta 4-dehydrogenase deficiency; 46, XY sex reversal, type 1, 3, and 5; 5-Oxoprolinase deficiency; 6-pyruvoyl-tetrahydropterin synthase deficiency; Aarskog syndrome; Aase syndrome; Achondrogenesis type 2; Achromatopsia 2 and 7; Acquired long QT syndrome; Acrocallosal syndrome, Schinzel type; Acrocapitofemoral dysplasia; Acrodysostosis 2, with or without hormone resistance; Acroerythrokeratoderma; Acromicric dysplasia; Acth-independent macronodular adrenal hyperplasia 2; Activated PI3K-delta syndrome; Acute intermittent porphyria; deficiency of Acyl-CoA dehydrogenase family, member 9; Adams-Oliver syndrome 5 and 6; Adenine phosphoribosyltransferase deficiency; Adenylate kinase deficiency; hemolytic anemia due to Adenylosuccinate lyase deficiency; Adolescent nephronophthisis; Renal-hepatic-pancreatic dysplasia; Meckel syndrome type 7; Adrenoleukodystrophy; Adult junctional epidermolysis bullosa; Epidermolysis bullosa, junctional, localisata variant; Adult neuronal ceroid lipofuscinosis; Adult neuronal ceroid lipofuscinosis; Adult onset ataxia with oculomotor apraxia; ADULT syndrome; Afibrinogenemia and congenital Afibrinogenemia; autosomal recessive Agammaglobulinemia 2; Age-related macular degeneration 3, 6, 11, and 12; Aicardi Goutieres syndromes 1, 4, and 5; Chilbain lupus 1; Alagille syndromes 1 and 2; Alexander disease; Alkaptonuria; Allan-Herndon-Dudley syndrome; Alopecia universalis congenital; Alpers encephalopathy; Alpha-1-antitrypsin deficiency; autosomal dominant, autosomal recessive, and X-linked recessive Alport syndromes; Alzheimer disease, familial, 3, with spastic paraparesis and apraxia; Alzheimer disease, types, 1, 3, and 4; hypocalcification type and hypomaturation type, IIA1 Amelogenesis imperfecta; Aminoacylase 1 deficiency; Amish infantile epilepsy syndrome; Amyloidogenic transthyretin amyloidosis; Amyloid Cardiomyopathy, Transthyretin-related; Cardiomyopathy; Amyotrophic lateral sclerosis types 1, 6, 15 (with or without frontotemporal dementia), 22 (with or without frontotemporal dementia), and 10; Frontotemporal dementia with TDP43 inclusions, TARDBP-related; Andermann syndrome; Andersen Tawil syndrome; Congenital long QT syndrome; Anemia, nonspherocytic hemolytic, due to G6PD deficiency; Angelman syndrome; Severe neonatal-onset encephalopathy with microcephaly; susceptibility to Autism, X-linked 3; Angiopathy, hereditary, with nephropathy, aneurysms, and muscle cramps; Angiotensin i-converting enzyme, benign serum increase; Aniridia, cerebellar ataxia, and mental retardation; Anonychia; Antithrombin III deficiency; Antley-Bixler syndrome with genital anomalies and disordered steroidogenesis; Aortic aneurysm, familial thoracic 4, 6, and 9; Thoracic aortic aneurysms and aortic dissections; Multisystemic smooth muscle dysfunction syndrome; Moyamoya disease 5; Aplastic anemia; Apparent mineralocorticoid excess; Arginase deficiency; Argininosuccinate lyase deficiency; Aromatase deficiency; Arrhythmogenic right ventricular cardiomyopathy types 5, 8, and 10; Primary familial hypertrophic cardiomyopathy; Arthrogryposis multiplex congenita, distal, X-linked; Arthrogryposis renal dysfunction cholestasis syndrome; Arthrogryposis, renal dysfunction, and cholestasis 2; Asparagine synthetase deficiency; Abnormality of neuronal migration; Ataxia with vitamin E deficiency; Ataxia, sensory, autosomal dominant; Ataxia-telangiectasia syndrome; Hereditary cancer-predisposing syndrome; Atransferrinemia; Atrial fibrillation, familial, 11, 12, 13, and 16; Atrial septal defects 2, 4, and 7 (with or without atrioventricular conduction defects); Atrial standstill 2; Atrioventricular septal defect 4; Atrophia bulborum hereditaria; ATR-X syndrome; Auriculocondylar syndrome 2; Autoimmune disease, multisystem, infantile-onset; Autoimmune lymphoproliferative syndrome, type 1a; Autosomal dominant hypohidrotic ectodermal dysplasia; Autosomal dominant progressive external ophthalmoplegia with mitochondrial DNA deletions 1 and 3; Autosomal dominant torsion dystonia 4; Autosomal recessive centronuclear myopathy; Autosomal recessive congenital ichthyosis 1, 2, 3, 4A, and 4B; Autosomal recessive cutis laxa type IA and 1B; Autosomal recessive hypohidrotic ectodermal dysplasia syndrome; Ectodermal dysplasia 11b; hypohidrotic/hair/tooth type, autosomal recessive; Autosomal recessive hypophosphatemic bone disease; Axenfeld-Rieger syndrome type 3; Bainbridge-Ropers syndrome; Bannayan-Riley-Ruvalcaba syndrome; PTEN hamartoma tumor syndrome; Baraitser-Winter syndromes 1 and 2; Barakat syndrome; Bardet-Biedl syndromes 1, 11, 16, and 19; Bare lymphocyte syndrome type 2, complementation group E; Bartter syndrome antenatal type 2; Bartter syndrome types 3, 3 with hypocalciuria, and 4; Basal ganglia calcification, idiopathic, 4; Beaded hair; Benign familial hematuria; Benign familial neonatal seizures 1 and 2; Seizures, benign familial neonatal, 1, and/or myokymia; Seizures, Early infantile epileptic encephalopathy 7; Benign familial neonatal-infantile seizures; Benign hereditary chorea; Benign scapuloperoneal muscular dystrophy with cardiomyopathy; Bernard-Soulier syndrome, types A1 and A2 (autosomal dominant); Bestrophinopathy, autosomal recessive; beta Thalassemia; Bethlem myopathy and Bethlem myopathy 2; Bietti crystalline corneoretinal dystrophy; Bile acid synthesis defect, congenital, 2; Biotinidase deficiency; Birk Barel mental retardation dysmorphism syndrome; Blepharophimosis, ptosis, and epicanthus inversus; Bloom syndrome; Borjeson-Forssman-Lehmann syndrome; Boucher Neuhauser syndrome; Brachydactyly types A1 and A2; Brachydactyly with hypertension; Brain small vessel disease with hemorrhage; Branched-chain ketoacid dehydrogenase kinase deficiency; Branchiootic syndromes 2 and 3; Breast cancer, early-onset; Breast-ovarian cancer, familial 1, 2, and 4; Brittle cornea syndrome 2; Brody myopathy; Bronchiectasis with or without elevated sweat chloride 3; Brown-Vialetto-Van laere syndrome and Brown-Vialetto-Van Laere syndrome 2; Brugada syndrome; Brugada syndrome 1; Ventricular fibrillation; Paroxysmal familial ventricular fibrillation; Brugada syndrome and Brugada syndrome 4; Long QT syndrome; Sudden cardiac death; Bull eye macular dystrophy; Stargardt disease 4; Cone-rod dystrophy 12; Bullous ichthyosiform erythroderma; Burn-Mckeown syndrome; Candidiasis, familial, 2, 5, 6, and 8; Carbohydrate-deficient glycoprotein syndrome type I and II; Carbonic anhydrase VA deficiency, hyperammonemia due to; Carcinoma of colon; Cardiac arrhythmia; Long QT syndrome, LQT1 subtype; Cardioencephalomyopathy, fatal infantile, due to cytochrome c oxidase deficiency; Cardiofaciocutaneous syndrome; Cardiomyopathy; Danon disease; Hypertrophic cardiomyopathy; Left ventricular noncompaction cardiomyopathy; Carnevale syndrome; Carney complex, type 1; Carnitine acylcarnitine translocase deficiency; Carnitine palmitoyltransferase I, II, II (late onset), and II (infantile) deficiency; Cataract 1, 4, autosomal dominant, autosomal dominant, multiple types, with microcornea, coppock-like, juvenile, with microcornea and glucosuria, and nuclear diffuse nonprogressive; Catecholaminergic polymorphic ventricular tachycardia; Caudal regression syndrome; Cd8 deficiency, familial; Central core disease; Centromeric instability of chromosomes 1,9 and 16 and immunodeficiency; Cerebellar ataxia infantile with progressive external ophthalmoplegi and Cerebellar ataxia, mental retardation, and dysequilibrium syndrome 2; Cerebral amyloid angiopathy, APP-related; Cerebral autosomal dominant and recessive arteriopathy with subcortical infarcts and leukoencephalopathy; Cerebral cavernous malformations 2; Cerebrooculofacioskeletal syndrome 2; Cerebro-oculo-facio-skeletal syndrome; Cerebroretinal microangiopathy with calcifications and cysts; Ceroid lipofuscinosis neuronal 2, 6, 7, and 10; Ch\xc3\xa9diak-Higashi syndrome, Chediak-Higashi syndrome, adult type; Charcot-Marie-Tooth disease types 1B, 2B2, 2C, 2F, 2I, 2U (axonal), 1C (demyelinating), dominant intermediate C, recessive intermediate A, 2A2, 4C, 4D, 4H, IF, IVF, and X; Scapuloperoneal spinal muscular atrophy; Distal spinal muscular atrophy, congenital nonprogressive; Spinal muscular atrophy, distal, autosomal recessive, 5; CHARGE association; Childhood hypophosphatasia; Adult hypophosphatasia; Cholecystitis; Progressive familial intrahepatic cholestasis 3; Cholestasis, intrahepatic, of pregnancy 3; Cholestanol storage disease; Cholesterol monooxygenase (side-chain cleaving) deficiency; Chondrodysplasia Blomstrand type; Chondrodysplasia punctata 1, X-linked recessive and 2 X-linked dominant; CHOPS syndrome; Chronic granulomatous disease, autosomal recessive cytochrome b-positive, types 1 and 2; Chudley-McCullough syndrome; Ciliary dyskinesia, primary, 7, 11, 15, 20 and 22; Citrullinemia type I; Citrullinemia type I and II; Cleidocranial dysostosis; C-like syndrome; Cockayne syndrome type A; Coenzyme Q10 deficiency, primary 1, 4, and 7; Coffin Siris/Intellectual Disability; Coffin-Lowry syndrome; Cohen syndrome; Cold-induced sweating syndrome 1; COLE-CARPENTER SYNDROME 2; Combined cellular and humoral immune defects with granulomas; Combined d-2- and 1-2-hydroxyglutaric aciduria; Combined malonic and methylmalonic aciduria; Combined oxidative phosphorylation deficiencies 1, 3, 4, 12, 15, and 25; Combined partial and complete 17-alpha-hydroxylase/17,20-lyase deficiency; Common variable immunodeficiency 9; Complement component 4, partial deficiency of, due to dysfunctional c1 inhibitor; Complement factor B deficiency; Cone monochromatism; Cone-rod dystrophy 2 and 6; Cone-rod dystrophy amelogenesis imperfecta; Congenital adrenal hyperplasia and Congenital adrenal hypoplasia, X-linked; Congenital amegakaryocytic thrombocytopenia; Congenital aniridia; Congenital central hypoventilation; Hirschsprung disease 3; Congenital contractural arachnodactyly; Congenital contractures of the limbs and face, hypotonia, and developmental delay; Congenital disorder of glycosylation types 1B, 1D, 1G, 1H, 1J, 1K, 1N, 1P, 2C, 2J, 2K, IIm; Congenital dyserythropoietic anemia, type I and II; Congenital ectodermal dysplasia of face; Congenital erythropoietic porphyria; Congenital generalized lipodystrophy type 2; Congenital heart disease, multiple types, 2; Congenital heart disease; Interrupted aortic arch; Congenital lipomatous overgrowth, vascular malformations, and epidermal nevi; Non-small cell lung cancer; Neoplasm of ovary; Cardiac conduction defect, nonspecific; Congenital microvillous atrophy; Congenital muscular dystrophy; Congenital muscular dystrophy due to partial LAMA2 deficiency; Congenital muscular dystrophy-dystroglycanopathy with brain and eye anomalies, types A2, A7, A8, All, and A14; Congenital muscular dystrophy-dystroglycanopathy with mental retardation, types B2, B3, B5, and B15; Congenital muscular dystrophy-dystroglycanopathy without mental retardation, type B5; Congenital muscular hypertrophy-cerebral syndrome; Congenital myasthenic syndrome, acetazolamide-responsive; Congenital myopathy with fiber type disproportion; Congenital ocular coloboma; Congenital stationary night blindness, type 1A, 1B, 1C, 1E, 1F, and 2A; Coproporphyria; Cornea plana 2; Corneal dystrophy, Fuchs endothelial, 4; Corneal endothelial dystrophy type 2; Corneal fragility keratoglobus, blue sclerae and joint hypermobility; Cornelia de Lange syndromes 1 and 5; Coronary artery disease, autosomal dominant 2; Coronary heart disease; Hyperalphalipoproteinemia 2; Cortical dysplasia, complex, with other brain malformations 5 and 6; Cortical malformations, occipital; Corticosteroid-binding globulin deficiency; Corticosterone methyloxidase type 2 deficiency; Costello syndrome; Cowden syndrome 1; Coxa plana; Craniodiaphyseal dysplasia, autosomal dominant; Craniosynostosis 1 and 4; Craniosynostosis and dental anomalies; Creatine deficiency, X-linked; Crouzon syndrome; Cryptophthalmos syndrome; Cryptorchidism, unilateral or bilateral; Cushing symphalangism; Cutaneous malignant melanoma 1; Cutis laxa with osteodystrophy and with severe pulmonary, gastrointestinal, and urinary abnormalities; Cyanosis, transient neonatal and atypical nephropathic; Cystic fibrosis; Cystinuria; Cytochrome c oxidase i deficiency; Cytochrome-c oxidase deficiency; D-2-hydroxyglutaric aciduria 2; Darier disease, segmental; Deafness with labyrinthine aplasia microtia and microdontia (LAMM); Deafness, autosomal dominant 3a, 4, 12, 13, 15, autosomal dominant nonsyndromic sensorineural 17, 20, and 65; Deafness, autosomal recessive 1A, 2, 3, 6, 8, 9, 12, 15, 16, 18b, 22, 28, 31, 44, 49, 63, 77, 86, and 89; Deafness, cochlear, with myopia and intellectual impairment, without vestibular involvement, autosomal dominant, X-linked 2; Deficiency of 2-methylbutyryl-CoA dehydrogenase; Deficiency of 3-hydroxyacyl-CoA dehydrogenase; Deficiency of alpha-mannosidase; Deficiency of aromatic-L-amino-acid decarboxylase; Deficiency of bisphosphoglycerate mutase; Deficiency of butyryl-CoA dehydrogenase; Deficiency of ferroxidase; Deficiency of galactokinase; Deficiency of guanidinoacetate methyltransferase; Deficiency of hyaluronoglucosaminidase; Deficiency of ribose-5-phosphate isomerase; Deficiency of steroid 11-beta-monooxygenase; Deficiency of UDPglucose-hexose-1-phosphate uridylyltransferase; Deficiency of xanthine oxidase; Dejerine-Sottas disease; Charcot-Marie-Tooth disease, types ID and IVF; Dejerine-Sottas syndrome, autosomal dominant; Dendritic cell, monocyte, B lymphocyte, and natural killer lymphocyte deficiency; Desbuquois dysplasia 2; Desbuquois syndrome; DFNA 2 Nonsyndromic Hearing Loss; Diabetes mellitus and insipidus with optic atrophy and deafness; Diabetes mellitus, type 2, and insulin-dependent, 20; Diamond-Blackfan anemia 1, 5, 8, and 10; Diarrhea 3 (secretory sodium, congenital, syndromic) and 5 (with tufting enteropathy, congenital); Dicarboxylic aminoaciduria; Diffuse palmoplantar keratoderma, Bothnian type; Digitorenocerebral syndrome; Dihydropteridine reductase deficiency; Dilated cardiomyopathy 1A, 1AA, 1C, 1G, 1BB, 1DD, 1FF, 1HH, 11, 1KK, 1N, 1S, 1Y, and 3B; Left ventricular noncompaction 3; Disordered steroidogenesis due to cytochrome p450 oxidoreductase deficiency; Distal arthrogryposis type 2B; Distal hereditary motor neuronopathy type 2B; Distal myopathy Markesbery-Griggs type; Distal spinal muscular atrophy, X-linked 3; Distichiasis-lymphedema syndrome; Dominant dystrophic epidermolysis bullosa with absence of skin; Dominant hereditary optic atrophy; Donnai Barrow syndrome; Dopamine beta hydroxylase deficiency; Dopamine receptor d2, reduced brain density of; Dowling-degos disease 4; Doyne honeycomb retinal dystrophy; Malattia leventinese; Duane syndrome type 2; Dubin-Johnson syndrome; Duchenne muscular dystrophy; Becker muscular dystrophy; Dysfibrinogenemia; Dyskeratosis congenita autosomal dominant and autosomal dominant, 3; Dyskeratosis congenita, autosomal recessive, 1, 3, 4, and 5; Dyskeratosis congenita X-linked; Dyskinesia, familial, with facial myokymia; Dysplasminogenemia; Dystonia 2 (torsion, autosomal recessive), 3 (torsion, X-linked), 5 (Dopa-responsive type), 10, 12, 16, 25, 26 (Myoclonic); Seizures, benign familial infantile, 2; Early infantile epileptic encephalopathy 2, 4, 7, 9, 10, 11, 13, and 14; Atypical Rett syndrome; Early T cell progenitor acute lymphoblastic leukemia; Ectodermal dysplasia skin fragility syndrome; Ectodermal dysplasia-syndactyly syndrome 1; Ectopia lentis, isolated autosomal recessive and dominant; Ectrodactyly, ectodermal dysplasia, and cleft lip/palate syndrome 3; Ehlers-Danlos syndrome type 7 (autosomal recessive), classic type, type 2 (progeroid), hydroxylysine-deficient, type 4, type 4 variant, and due to tenascin-X deficiency; Eichsfeld type congenital muscular dystrophy; Endocrine-cerebroosteodysplasia; Enhanced s-cone syndrome; Enlarged vestibular aqueduct syndrome; Enterokinase deficiency; Epidermodysplasia verruciformis; Epidermolysa bullosa simplex and limb girdle muscular dystrophy, simplex with mottled pigmentation, simplex with pyloric atresia, simplex, autosomal recessive, and with pyloric atresia; Epidermolytic palmoplantar keratoderma; Familial febrile seizures 8; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Epileptic encephalopathy, childhood-onset, early infantile, 1, 19, 23, 25, 30, and 32; Epiphyseal dysplasia, multiple, with myopia and conductive deafness; Episodic ataxia type 2; Episodic pain syndrome, familial, 3; Epstein syndrome; Fechtner syndrome; Erythropoietic protoporphyria; Estrogen resistance; Exudative vitreoretinopathy 6; Fabry disease and Fabry disease, cardiac variant; Factor H, VII, X, v and factor viii, combined deficiency of 2, xiii, a subunit, deficiency; Familial adenomatous polyposis 1 and 3; Familial amyloid nephropathy with urticaria and deafness; Familial cold urticarial; Familial aplasia of the vermis; Familial benign pemphigus; Familial cancer of breast; Breast cancer, susceptibility to; Osteosarcoma; Pancreatic cancer 3; Familial cardiomyopathy; Familial cold autoinflammatory syndrome 2; Familial colorectal cancer; Familial exudative vitreoretinopathy, X-linked; Familial hemiplegic migraine types 1 and 2; Familial hypercholesterolemia; Familial hypertrophic cardiomyopathy 1, 2, 3, 4, 7, 10, 23 and 24; Familial hypokalemia-hypomagnesemia; Familial hypoplastic, glomerulocystic kidney; Familial infantile myasthenia; Familial juvenile gout; Familial Mediterranean fever and Familial mediterranean fever, autosomal dominant; Familial porencephaly; Familial Porphyria cutanea tarda; Familial pulmonary capillary hemangiomatosis; Familial renal glucosuria; Familial renal hypouricemia; Familial restrictive cardiomyopathy 1; Familial type 1 and 3 hyperlipoproteinemia; Fanconi anemia, complementation group E, I, N, and O; Fanconi-Bickel syndrome; Favism, susceptibility to; Febrile seizures, familial, 11; Feingold syndrome 1; Fetal hemoglobin quantitative trait locus 1; FG syndrome and FG syndrome 4; Fibrosis of extraocular muscles, congenital, 1, 2, 3a (with or without extraocular involvement), 3b; Fish-eye disease; Fleck corneal dystrophy; Floating-Harbor syndrome; Focal epilepsy with speech disorder with or without mental retardation; Focal segmental glomerulosclerosis 5; Forebrain defects; Frank Ter Haar syndrome; Borrone Di Rocco Crovato syndrome; Frasier syndrome; Wilms tumor 1; Freeman-Sheldon syndrome; Frontometaphyseal dysplasia land 3; Frontotemporal dementia; Frontotemporal dementia and/or amyotrophic lateral sclerosis 3 and 4; Frontotemporal Dementia Chromosome 3-Linked and Frontotemporal dementia ubiquitin-positive; Fructose-biphosphatase deficiency; Fuhrmann syndrome; Gamma-aminobutyric acid transaminase deficiency; Gamstorp-Wohlfart syndrome; Gaucher disease type 1 and Subacute neuronopathic; Gaze palsy, familial horizontal, with progressive scoliosis; Generalized dominant dystrophic epidermolysis bullosa; Generalized epilepsy with febrile seizures plus 3, type 1, type 2; Epileptic encephalopathy Lennox-Gastaut type; Giant axonal neuropathy; Glanzmann thrombasthenia; Glaucoma 1, open angle, e, F, and G; Glaucoma 3, primary congenital, d; Glaucoma, congenital and Glaucoma, congenital, Coloboma; Glaucoma, primary open angle, juvenile-onset; Glioma susceptibility 1; Glucose transporter type 1 deficiency syndrome; Glucose-6-phosphate transport defect; GLUT1 deficiency syndrome 2; Epilepsy, idiopathic generalized, susceptibility to, 12; Glutamate formiminotransferase deficiency; Glutaric acidemia IIA and IIB; Glutaric aciduria, type 1; Gluthathione synthetase deficiency; Glycogen storage disease 0 (muscle), II (adult form), IXa2, IXc, type 1A; type II, type IV, IV (combined hepatic and myopathic), type V, and type VI; Goldmann-Favre syndrome; Gordon syndrome; Gorlin syndrome; Holoprosencephaly sequence; Holoprosencephaly 7; Granulomatous disease, chronic, X-linked, variant; Granulosa cell tumor of the ovary; Gray platelet syndrome; Griscelli syndrome type 3; Groenouw corneal dystrophy type I; Growth and mental retardation, mandibulofacial dysostosis, microcephaly, and cleft palate; Growth hormone deficiency with pituitary anomalies; Growth hormone insensitivity with immunodeficiency; GTP cyclohydrolase I deficiency; Hajdu-Cheney syndrome; Hand foot uterus syndrome; Hearing impairment; Hemangioma, capillary infantile; Hematologic neoplasm; Hemochromatosis type 1, 2B, and 3; Microvascular complications of diabetes 7; Transferrin serum level quantitative trait locus 2; Hemoglobin H disease, nondeletional; Hemolytic anemia, nonspherocytic, due to glucose phosphate isomerase deficiency; Hemophagocytic lymphohistiocytosis, familial, 2; Hemophagocytic lymphohistiocytosis, familial, 3; Heparin cofactor II deficiency; Hereditary acrodermatitis enteropathica; Hereditary breast and ovarian cancer syndrome; Ataxia-telangiectasia-like disorder; Hereditary diffuse gastric cancer; Hereditary diffuse leukoencephalopathy with spheroids; Hereditary factors II, IX, VIII deficiency disease; Hereditary hemorrhagic telangiectasia type 2; Hereditary insensitivity to pain with anhidrosis; Hereditary lymphedema type I; Hereditary motor and sensory neuropathy with optic atrophy; Hereditary myopathy with early respiratory failure; Hereditary neuralgic amyotrophy; Hereditary Nonpolyposis Colorectal Neoplasms; Lynch syndrome I and II; Hereditary pancreatitis; Pancreatitis, chronic, susceptibility to; Hereditary sensory and autonomic neuropathy type IIB amd IIA; Hereditary sideroblastic anemia; Hermansky-Pudlak syndrome 1, 3, 4, and 6; Heterotaxy, visceral, 2, 4, and 6, autosomal; Heterotaxy, visceral, X-linked; Heterotopia; Histiocytic medullary reticulosis; Histiocytosis-lymphadenopathy plus syndrome; Holocarboxylase synthetase deficiency; Holoprosencephaly 2, 3,7, and 9; Holt-Oram syndrome; Homocysteinemia due to MTHFR deficiency, CBS deficiency, and Homocystinuria, pyridoxine-responsive; Homocystinuria-Megaloblastic anemia due to defect in cobalamin metabolism, cblE complementation type; Howel-Evans syndrome; Hurler syndrome; Hutchinson-Gilford syndrome; Hydrocephalus; Hyperammonemia, type III; Hypercholesterolaemia and Hypercholesterolemia, autosomal recessive; Hyperekplexia 2 and Hyperekplexia hereditary; Hyperferritinemia cataract syndrome; Hyperglycinuria; Hyperimmunoglobulin D with periodic fever; Mevalonic aciduria; Hyperimmunoglobulin E syndrome; Hyperinsulinemic hypoglycemia familial 3, 4, and 5; Hyperinsulinism-hyperammonemia syndrome; Hyperlysinemia; Hypermanganesemia with dystonia, polycythemia and cirrhosis; Hyperornithinemia-hyperammonemia-homocitrullinuria syndrome; Hyperparathyroidism 1 and 2; Hyperparathyroidism, neonatal severe; Hyperphenylalaninemia, bh4-deficient, a, due to partial pts deficiency, BH4-deficient, D, and non-pku; Hyperphosphatasia with mental retardation syndrome 2, 3, and 4; Hypertrichotic osteochondrodysplasia; Hypobetalipoproteinemia, familial, associated with apob32; Hypocalcemia, autosomal dominant 1; Hypocalciuric hypercalcemia, familial, types 1 and 3; Hypochondrogenesis; Hypochromic microcytic anemia with iron overload; Hypoglycemia with deficiency of glycogen synthetase in the liver; Hypogonadotropic hypogonadism 11 with or without anosmia; Hypohidrotic ectodermal dysplasia with immune deficiency; Hypohidrotic X-linked ectodermal dysplasia; Hypokalemic periodic paralysis 1 and 2; Hypomagnesemia 1, intestinal; Hypomagnesemia, seizures, and mental retardation; Hypomyelinating leukodystrophy 7; Hypoplastic left heart syndrome; Atrioventricular septal defect and common atrioventricular junction; Hypospadias 1 and 2, X-linked; Hypothyroidism, congenital, nongoitrous, 1; Hypotrichosis 8 and 12; Hypotrichosis-lymphedema-telangiectasia syndrome; I blood group system; Ichthyosis bullosa of Siemens; Ichthyosis exfoliativa; Ichthyosis prematurity syndrome; Idiopathic basal ganglia calcification 5; Idiopathic fibrosing alveolitis, chronic form; Dyskeratosis congenita, autosomal dominant, 2 and 5; Idiopathic hypercalcemia of infancy; Immune dysfunction with T-cell inactivation due to calcium entry defect 2; Immunodeficiency 15, 16, 19, 30, 31C, 38, 40, 8, due to defect in cd3-zeta, with hyper IgM type 1 and 2, and X-Linked, with magnesium defect, Epstein-Barr virus infection, and neoplasia; Immunodeficiency-centromeric instability-facial anomalies syndrome 2; Inclusion body myopathy 2 and 3; Nonaka myopathy; Infantile convulsions and paroxysmal choreoathetosis, familial; Infantile cortical hyperostosis; Infantile GM1 gangliosidosis; Infantile hypophosphatasia; Infantile nephronophthisis; Infantile nystagmus, X-linked; Infantile Parkinsonism-dystonia; Infertility associated with multi-tailed spermatozoa and excessive DNA; Insulin resistance; Insulin-resistant diabetes mellitus and acanthosis nigricans; Insulin-dependent diabetes mellitus secretory diarrhea syndrome; Interstitial nephritis, karyomegalic; Intrauterine growth retardation, metaphyseal dysplasia, adrenal hypoplasia congenita, and genital anomalies; Iodotyrosyl coupling defect; IRAK4 deficiency; Iridogoniodysgenesis dominant type and type 1; Iron accumulation in brain; Ischiopatellar dysplasia; Islet cell hyperplasia; Isolated 17,20-lyase deficiency; Isolated lutropin deficiency; Isovaleryl-CoA dehydrogenase deficiency; Jankovic Rivera syndrome; Jervell and Lange-Nielsen syndrome 2; Joubert syndrome 1, 6, 7, 9/15 (digenic), 14, 16, and 17, and Orofaciodigital syndrome xiv; Junctional epidermolysis bullosa gravis of Herlitz; Juvenile GM>1<gangliosidosis; Juvenile polyposis syndrome; Juvenile polyposis/hereditary hemorrhagic telangiectasia syndrome; Juvenile retinoschisis; Kabuki make-up syndrome; Kallmann syndrome 1, 2, and 6; Delayed puberty; Kanzaki disease; Karak syndrome; Kartagener syndrome; Kenny-Caffey syndrome type 2; Keppen-Lubinsky syndrome; Keratoconus 1; Keratosis follicularis; Keratosis palmoplantaris striata 1; Kindler syndrome; L-2-hydroxyglutaric aciduria; Larsen syndrome, dominant type; Lattice corneal dystrophy Type III; Leber amaurosis; Zellweger syndrome; Peroxisome biogenesis disorders; Zellweger syndrome spectrum; Leber congenital amaurosis 11, 12, 13, 16, 4, 7, and 9; Leber optic atrophy; Aminoglycoside-induced deafness; Deafness, nonsyndromic sensorineural, mitochondrial; Left ventricular noncompaction 5; Left-right axis malformations; Leigh disease; Mitochondrial short-chain Enoyl-CoA Hydratase 1 deficiency; Leigh syndrome due to mitochondrial complex I deficiency; Leiner disease; Leri Weill dyschondrosteosis; Lethal congenital contracture syndrome 6; Leukocyte adhesion deficiency type I and III; Leukodystrophy, Hypomyelinating, 11 and 6; Leukoencephalopathy with ataxia, with Brainstem and Spinal Cord Involvement and Lactate Elevation, with vanishing white matter, and progressive, with ovarian failure; Leukonychia totalis; Lewy body dementia; Lichtenstein-Knorr Syndrome; Li-Fraumeni syndrome 1; Lig4 syndrome; Limb-girdle muscular dystrophy, type 1B, 2A, 2B, 2D, C1, C5, C9, C14; Congenital muscular dystrophy-dystroglycanopathy with brain and eye anomalies, type A14 and B14; Lipase deficiency combined; Lipid proteinosis; Lipodystrophy, familial partial, type 2 and 3; Lissencephaly 1, 2 (X-linked), 3, 6 (with microcephaly), X-linked; Subcortical laminar heterotopia, X-linked; Liver failure acute infantile; Loeys-Dietz syndrome 1, 2, 3; Long QT syndrome 1, 2, 2/9, 2/5, (digenic), 3, 5 and 5, acquired, susceptibility to; Lung cancer; Lymphedema, hereditary, id; Lymphedema, primary, with myelodysplasia; Lymphoproliferative syndrome 1, 1 (X-linked), and 2; Lysosomal acid lipase deficiency; Macrocephaly, macrosomia, facial dysmorphism syndrome; Macular dystrophy, vitelliform, adult-onset; Malignant hyperthermia susceptibility type 1; Malignant lymphoma, non-Hodgkin; Malignant melanoma; Malignant tumor of prostate; Mandibuloacral dysostosis; Mandibuloacral dysplasia with type A or B lipodystrophy, atypical; Mandibulofacial dysostosis, Treacher Collins type, autosomal recessive; Mannose-binding protein deficiency; Maple syrup urine disease type lA and type 3; Marden Walker like syndrome; Marfan syndrome; Marinesco-Sjxc3xb6gren syndrome; Martsolf syndrome; Maturity-onset diabetes of the young, type 1, type 2, type 11, type 3, and type 9; May-Hegglin anomaly; MYH9 related disorders; Sebastian syndrome; McCune-Albright syndrome; Somatotroph adenoma; Sex cord-stromal tumor; Cushing syndrome; McKusick Kaufman syndrome; McLeod neuroacanthocytosis syndrome; Meckel-Gruber syndrome; Medium-chain acyl-coenzyme A dehydrogenase deficiency; Medulloblastoma; Megalencephalic leukoencephalopathy with subcortical cysts land 2a; Megalencephaly cutis marmorata telangiectatica congenital; PIK3CA Related Overgrowth Spectrum; Megalencephaly-polymicrogyria-polydactyly-hydrocephalus syndrome 2; Megaloblastic anemia, thiamine-responsive, with diabetes mellitus and sensorineural deafness; Meier-Gorlin syndromes land 4; Melnick-Needles syndrome; Meningioma; Mental retardation, X-linked, 3, 21, 30, and 72; Mental retardation and microcephaly with pontine and cerebellar hypoplasia; Mental retardation X-linked syndromic 5; Mental retardation, anterior maxillary protrusion, and strabismus; Mental retardation, autosomal dominant 12, 13, 15, 24, 3, 30, 4, 5, 6,and 9; Mental retardation, autosomal recessive 15, 44, 46, and 5; Mental retardation, stereotypic movements, epilepsy, and/or cerebral malformations; Mental retardation, syndromic, Claes-Jensen type, X-linked; Mental retardation, X-linked, nonspecific, syndromic, Hedera type, and syndromic, wu type; Merosin deficient congenital muscular dystrophy; Metachromatic leukodystrophy juvenile, late infantile, and adult types; Metachromatic leukodystrophy; Metatrophic dysplasia; Methemoglobinemia types I and 2; Methionine adenosyltransferase deficiency, autosomal dominant; Methylmalonic acidemia with homocystinuria; Methylmalonic aciduria cblB type; Methylmalonic aciduria due to methylmalonyl-CoA mutase deficiency; Methylmalonic aciduria, mut(0) type; Microcephalic osteodysplastic primordial dwarfism type 2; Microcephaly with or without chorioretinopathy, lymphedema, or mental retardation; Microcephaly, hiatal hernia and nephrotic syndrome; Microcephaly; Hypoplasia of the corpus callosum; Spastic paraplegia 50, autosomal recessive; Global developmental delay; CNS hypomyelination; Brain atrophy; Microcephaly, normal intelligence and immunodeficiency; Microcephaly-capillary malformation syndrome; Microcytic anemia; Microphthalmia syndromic 5, 7, and 9; Microphthalmia, isolated 3, 5, 6, 8, and with coloboma 6; Microspherophakia; Migraine, familial basilar; Miller syndrome; Minicore myopathy with external ophthalmoplegia; Myopathy, congenital with cores; Mitchell-Riley syndrome; mitochondrial 3-hydroxy-3-methylglutaryl-CoA synthase deficiency; Mitochondrial complex I, II, III, III (nuclear type 2, 4, or 8) deficiency; Mitochondrial DNA depletion syndrome 11, 12 (cardiomyopathic type), 2, 4B (MNGIE type), 8B (MNGIE type); Mitochondrial DNA-depletion syndrome 3 and 7, hepatocerebral types, and 13 (encephalomyopathic type); Mitochondrial phosphate carrier and pyruvate carrier deficiency; Mitochondrial trifunctional protein deficiency; Long-chain 3-hydroxyacyl-CoA dehydrogenase deficiency; Miyoshi muscular dystrophy 1; Myopathy, distal, with anterior tibial onset; Mohr-Tranebjaerg syndrome; Molybdenum cofactor deficiency, complementation group A; Mowat-Wilson syndrome; Mucolipidosis III Gamma; Mucopolysaccharidosis type VI, type VI (severe), and type VII; Mucopolysaccharidosis, MPS-I-H/S, MPS-II, MPS-III-A, MPS-III-B, MPS-III-C, MPS-IV-A, MPS-IV-B; Retinitis Pigmentosa 73; Gangliosidosis GM1 typel (with cardiac involvenment) 3; Multicentric osteolysis nephropathy; Multicentric osteolysis, nodulosis and arthropathy; Multiple congenital anomalies; Atrial septal defect 2; Multiple congenital anomalies-hypotonia-seizures syndrome 3; Multiple Cutaneous and Mucosal Venous Malformations; Multiple endocrine neoplasia, types land 4; Multiple epiphyseal dysplasia 5 or Dominant; Multiple gastrointestinal atresias; Multiple pterygium syndrome Escobar type; Multiple sulfatase deficiency; Multiple synostoses syndrome 3; Muscle AMP deaminase deficiency; Muscle eye brain disease; Muscular dystrophy, congenital, megaconial type; Myasthenia, familial infantile, 1; Myasthenic Syndrome, Congenital, 11, associated with acetylcholine receptor deficiency; Myasthenic Syndrome, Congenital, 17, 2A (slow-channel), 4B (fast-channel), and without tubular aggregates; Myeloperoxidase deficiency; MYH-associated polyposis; Endometrial carcinoma; Myocardial infarction 1; Myoclonic dystonia; Myoclonic-Atonic Epilepsy; Myoclonus with epilepsy with ragged red fibers; Myofibrillar myopathy 1 and ZASP-related; Myoglobinuria, acute recurrent, autosomal recessive; Myoneural gastrointestinal encephalopathy syndrome; Cerebellar ataxia infantile with progressive external ophthalmoplegia; Mitochondrial DNA depletion syndrome 4B, MNGIE type; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Myopia 6; Myosclerosis, autosomal recessive; Myotonia congenital; Congenital myotonia, autosomal dominant and recessive forms; Nail-patella syndrome; Nance-Horan syndrome; Nanophthalmos 2; Navajo neurohepatopathy; Nemaline myopathy 3 and 9; Neonatal hypotonia; Intellectual disability; Seizures; Delayed speech and language development; Mental retardation, autosomal dominant 31; Neonatal intrahepatic cholestasis caused by citrin deficiency; Nephrogenic diabetes insipidus, Nephrogenic diabetes insipidus, X-linked; Nephrolithiasis/osteoporosis, hypophosphatemic, 2; Nephronophthisis 13, 15 and 4; Infertility; Cerebello-oculo-renal syndrome (nephronophthisis, oculomotor apraxia and cerebellar abnormalities); Nephrotic syndrome, type 3, type 5, with or without ocular abnormalities, type 7, and type 9; Nestor-Guillermo progeria syndrome; Neu-Laxova syndrome 1; Neurodegeneration with brain iron accumulation 4 and 6; Neuroferritinopathy; Neurofibromatosis, type land type 2; Neurofibrosarcoma; Neurohypophyseal diabetes insipidus; Neuropathy, Hereditary Sensory, Type IC; Neutral 1 amino acid transport defect; Neutral lipid storage disease with myopathy; Neutrophil immunodeficiency syndrome; Nicolaides-Baraitser syndrome; Niemann-Pick disease type C1, C2, type A, and type C1, adult form; Non-ketotic hyperglycinemia; Noonan syndrome 1 and 4, LEOPARD syndrome 1; Noonan syndrome-like disorder with or without juvenile myelomonocytic leukemia; Normokalemic periodic paralysis, potassium-sensitive; Norum disease; Epilepsy, Hearing Loss, And Mental Retardation Syndrome; Mental Retardation, X-Linked 102 and syndromic 13; Obesity; Ocular albinism, type I; Oculocutaneous albinism type 1B, type 3, and type 4; Oculodentodigital dysplasia; Odontohypophosphatasia; Odontotrichomelic syndrome; Oguchi disease; Oligodontia-colorectal cancer syndrome; Opitz G/BBB syndrome; Optic atrophy 9; Oral-facial-digital syndrome; Ornithine aminotransferase deficiency; Orofacial cleft 11 and 7, Cleft lip/palate-ectodermal dysplasia syndrome; Orstavik Lindemann Solberg syndrome; Osteoarthritis with mild chondrodysplasia; Osteochondritis dissecans; Osteogenesis imperfecta type 12, type 5, type 7, type 8, type I, type III, with normal sclerae, dominant form, recessive perinatal lethal; Osteopathia striata with cranial sclerosis; Osteopetrosis autosomal dominant type 1 and 2, recessive 4, recessive 1, recessive 6; Osteoporosis with pseudoglioma; Oto-palato-digital syndrome, types I and II; Ovarian dysgenesis 1; Ovarioleukodystrophy; Pachyonychia congenita 4 and type 2; Paget disease of bone, familial; Pallister-Hall syndrome; Palmoplantar keratoderma, nonepidermolytic, focal or diffuse; Pancreatic agenesis and congenital heart disease; Papillon-Lef\xc3\xa8vre syndrome; Paragangliomas 3; Paramyotonia congenita of von Eulenburg; Parathyroid carcinoma; Parkinson disease 14, 15, 19 (juvenile-onset), 2, 20 (early-onset), 6, (autosomal recessive early-onset, and 9; Partial albinism; Partial hypoxanthine-guanine phosphoribosyltransferase deficiency; Patterned dystrophy of retinal pigment epithelium; PC-K6a; Pelizaeus-Merzbacher disease; Pendred syndrome; Peripheral demyelinating neuropathy, central dysmyelination; Hirschsprung disease; Permanent neonatal diabetes mellitus; Diabetes mellitus, permanent neonatal, with neurologic features; Neonatal insulin-dependent diabetes mellitus; Maturity-onset diabetes of the young, type 2; Peroxisome biogenesis disorder 14B, 2A, 4A, 5B, 6A, 7A, and 7B; Perrault syndrome 4; Perry syndrome; Persistent hyperinsulinemic hypoglycemia of infancy; familial hyperinsulinism; Phenotypes; Phenylketonuria; Pheochromocytoma; Hereditary Paraganglioma-Pheochromocytoma Syndromes; Paragangliomas 1; Carcinoid tumor of intestine; Cowden syndrome 3; Phosphoglycerate dehydrogenase deficiency; Phosphoglycerate kinase 1 deficiency; Photosensitive trichothiodystrophy; Phytanic acid storage disease; Pick disease; Pierson syndrome; Pigmentary retinal dystrophy; Pigmented nodular adrenocortical disease, primary, 1; Pilomatrixoma; Pitt-Hopkins syndrome; Pituitary dependent hypercortisolism; Pituitary hormone deficiency, combined 1, 2, 3, and 4; Plasminogen activator inhibitor type 1 deficiency; Plasminogen deficiency, type I; Platelet-type bleeding disorder 15 and 8; Poikiloderma, hereditary fibrosing, with tendon contractures, myopathy, and pulmonary fibrosis; Polycystic kidney disease 2, adult type, and infantile type; Polycystic lipomembranous osteodysplasia with sclerosing leukoencephalopathy; Polyglucosan body myopathy 1 with or without immunodeficiency; Polymicrogyria, asymmetric, bilateral frontoparietal; Polyneuropathy, hearing loss, ataxia, retinitis pigmentosa, and cataract; Pontocerebellar hypoplasia type 4; Popliteal pterygium syndrome; Porencephaly 2; Porokeratosis 8, disseminated superficial actinic type; Porphobilinogen synthase deficiency; Porphyria cutanea tarda; Posterior column ataxia with retinitis pigmentosa; Posterior polar cataract type 2; Prader-Willi-like syndrome; Premature ovarian failure 4, 5, 7, and 9; Primary autosomal recessive microcephaly 10, 2, 3, and 5; Primary ciliary dyskinesia 24; Primary dilated cardiomyopathy; Left ventricular noncompaction 6; 4, Left ventricular noncompaction 10; Paroxysmal atrial fibrillation; Primary hyperoxaluria, type I, type, and type III; Primary hypertrophic osteoarthropathy, autosomal recessive 2; Primary hypomagnesemia; Primary open angle glaucoma juvenile onset 1; Primary pulmonary hypertension; Primrose syndrome; Progressive familial heart block type 1B; Progressive familial intrahepatic cholestasis 2 and 3; Progressive intrahepatic cholestasis; Progressive myoclonus epilepsy with ataxia; Progressive pseudorheumatoid dysplasia; Progressive sclerosing poliodystrophy; Prolidase deficiency; Proline dehydrogenase deficiency; Schizophrenia 4; Properdin deficiency, X-linked; Propionic academia; Proprotein convertase 1/3 deficiency; Prostate cancer, hereditary, 2; Protan defect; Proteinuria; Finnish congenital nephrotic syndrome; Proteus syndrome; Breast adenocarcinoma; Pseudoachondroplastic spondyloepiphyseal dysplasia syndrome; Pseudohypoaldosteronism type 1 autosomal dominant and recessive and type 2; Pseudohypoparathyroidism type 1A, Pseudopseudohypoparathyroidism; Pseudoneonatal adrenoleukodystrophy; Pseudoprimary hyperaldosteronism; Pseudoxanthoma elasticum; Generalized arterial calcification of infancy 2; Pseudoxanthoma elasticum-like disorder with multiple coagulation factor deficiency; Psoriasis susceptibility 2; PTEN hamartoma tumor syndrome; Pulmonary arterial hypertension related to hereditary hemorrhagic telangiectasia; Pulmonary Fibrosis And/Or Bone Marrow Failure, Telomere-Related, 1 and 3; Pulmonary hypertension, primary, 1, with hereditary hemorrhagic telangiectasia; Purine-nucleoside phosphorylase deficiency; Pyruvate carboxylase deficiency; Pyruvate dehydrogenase El-alpha deficiency; Pyruvate kinase deficiency of red cells; Raine syndrome; Rasopathy; Recessive dystrophic epidermolysis bullosa; Nail disorder, nonsyndromic congenital, 8; Reifenstein syndrome; Renal adysplasia; Renal carnitine transport defect; Renal coloboma syndrome; Renal dysplasia; Renal dysplasia, retinal pigmentary dystrophy, cerebellar ataxia and skeletal dysplasia; Renal tubular acidosis, distal, autosomal recessive, with late-onset sensorineural hearing loss, or with hemolytic anemia; Renal tubular acidosis, proximal, with ocular abnormalities and mental retardation; Retinal cone dystrophy 3B; Retinitis pigmentosa; Retinitis pigmentosa 10, 11, 12, 14, 15, 17, and 19; Retinitis pigmentosa 2, 20, 25, 35, 36, 38, 39, 4, 40, 43, 45, 48, 66, 7, 70, 72; Retinoblastoma; Rett disorder; Rhabdoid tumor predisposition syndrome 2; Rhegmatogenous retinal detachment, autosomal dominant; Rhizomelic chondrodysplasia punctata type 2 and type 3; Roberts-SC phocomelia syndrome; Robinow Sorauf syndrome; Robinow syndrome, autosomal recessive, autosomal recessive, with brachy-syn-polydactyly; Rothmund-Thomson syndrome; Rapadilino syndrome; RRM2B-related mitochondrial disease; Rubinstein-Taybi syndrome; Salla disease; Sandhoff disease, adult and infantil types; Sarcoidosis, early-onset; Blau syndrome; Schindler disease, type 1; Schizencephaly; Schizophrenia 15; Schneckenbecken dysplasia; Schwannomatosis 2; Schwartz Jampel syndrome type 1; Sclerocornea, autosomal recessive; Sclerosteosis; Secondary hypothyroidism; Segawa syndrome, autosomal recessive; Senior-Loken syndrome 4 and 5; Sensory ataxic neuropathy, dysarthria, and ophthalmoparesis; Sepiapterin reductase deficiency; SeSAME syndrome; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Partial cytosine deaminase deficiency; Severe congenital neutropenia; Severe congenital neutropenia 3, autosomal recessive or dominant; Severe congenital neutropenia and 6, autosomal recessive; Severe myoclonic epilepsy in infancy; Generalized epilepsy with febrile seizures plus, types 1 and 2; Severe X-linked myotubular myopathy; Short QT syndrome 3; Short stature with nonspecific skeletal abnormalities; Short stature, auditory canal atresia, mandibular hypoplasia, skeletal abnormalities; Short stature, onychodysplasia, facial dysmorphism, and hypotrichosis; Primordial dwarfism; Short-rib thoracic dysplasia 11 or 3 with or without polydactyly; Sialidosis type I and II; Silver spastic paraplegia syndrome; Slowed nerve conduction velocity, autosomal dominant; Smith-Lemli-Opitz syndrome; Snyder Robinson syndrome; Somatotroph adenoma; Prolactinoma; familial, Pituitary adenoma predisposition; Sotos syndrome 1 or 2; Spastic ataxia 5, autosomal recessive, Charlevoix-Saguenay type, 1,10, or 11, autosomal recessive; Amyotrophic lateral sclerosis type 5; Spastic paraplegia 15, 2, 3, 35, 39, 4, autosomal dominant, 55, autosomal recessive, and 5A; Bile acid synthesis defect, congenital, 3; Spermatogenic failure 11, 3, and 8; Spherocytosis types 4 and 5; Spheroid body myopathy; Spinal muscular atrophy, lower extremity predominant 2, autosomal dominant; Spinal muscular atrophy, type II; Spinocerebellar ataxia 14, 21, 35, 40,and 6; Spinocerebellar ataxia autosomal recessive 1 and 16; Splenic hypoplasia; Spondylocarpotarsal synostosis syndrome; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Parastremmatic dwarfism; Stargardt disease 1; Cone-rod dystrophy 3; Stickler syndrome type 1; Kniest dysplasia; Stickler syndrome, types 1(nonsyndromic ocular) and 4; Sting-associated vasculopathy, infantile-onset; Stormorken syndrome; Sturge-Weber syndrome, Capillary malformations, congenital, 1; Succinyl-CoA acetoacetate transferase deficiency; Sucrase-isomaltase deficiency; Sudden infant death syndrome; Sulfite oxidase deficiency, isolated; Supravalvar aortic stenosis; Surfactant metabolism dysfunction, pulmonary, 2 and 3; Symphalangism, proximal, lb; Syndactyly Cenani Lenz type; Syndactyly type 3; Syndromic X-linked mental retardation 16; Talipes equinovarus; Tangier disease; TARP syndrome; Tay-Sachs disease, B1 variant, Gm2-gangliosidosis (adult), Gm2-gangliosidosis (adult-onset); Temtamy syndrome; Tenorio Syndrome; Terminal osseous dysplasia; Testosterone 17-beta-dehydrogenase deficiency; Tetraamelia, autosomal recessive; Tetralogy of Fallot; Hypoplastic left heart syndrome 2; Truncus arteriosus; Malformation of the heart and great vessels; Ventricular septal defect 1; Thiel-Behnke corneal dystrophy; Thoracic aortic aneurysms and aortic dissections; Marfanoid habitus; Three M syndrome 2; Thrombocytopenia, platelet dysfunction, hemolysis, and imbalanced globin synthesis; Thrombocytopenia, X-linked; Thrombophilia, hereditary, due to protein C deficiency, autosomal dominant and recessive; Thyroid agenesis; Thyroid cancer, follicular; Thyroid hormone metabolism, abnormal; Thyroid hormone resistance, generalized, autosomal dominant; Thyrotoxic periodic paralysis and Thyrotoxic periodic paralysis 2; Thyrotropin-releasing hormone resistance, generalized; Timothy syndrome; TNF receptor-associated periodic fever syndrome (TRAPS); Tooth agenesis, selective, 3 and 4; Torsades de pointes; Townes-Brocks-branchiootorenal-like syndrome; Transient bullous dermolysis of the newborn; Treacher collins syndrome 1; Trichomegaly with mental retardation, dwarfism and pigmentary degeneration of retina; Trichorhinophalangeal dysplasia type I; Trichorhinophalangeal syndrome type 3; Trimethylaminuria; Tuberous sclerosis syndrome; Lymphangiomyomatosis; Tuberous sclerosis 1 and 2; Tyrosinase-negative oculocutaneous albinism; Tyrosinase-positive oculocutaneous albinism; Tyrosinemia type I; UDPglucose-4-epimerase deficiency; Ullrich congenital muscular dystrophy; Ulna and fibula absence of with severe limb deficiency; Upshaw-Schulman syndrome; Urocanate hydratase deficiency; Usher syndrome, types 1, 1B, 1D, 1G, 2A, 2C, and 2D; Retinitis pigmentosa 39; UV-sensitive syndrome; Van der Woude syndrome; Van Maldergem syndrome 2; Hennekam lymphangiectasia-lymphedema syndrome 2; Variegate porphyria; Ventriculomegaly with cystic kidney disease; Verheij syndrome; Very long chain acyl-CoA dehydrogenase deficiency; Vesicoureteral reflux 8; Visceral heterotaxy 5, autosomal; Visceral myopathy; Vitamin D-dependent rickets, types land 2; Vitelliform dystrophy; von Willebrand disease type 2M and type 3; Waardenburg syndrome type 1, 4C, and 2E (with neurologic involvement); Klein-Waardenberg syndrome; Walker-Warburg congenital muscular dystrophy; Warburg micro syndrome 2 and 4; Warts, hypogammaglobulinemia, infections, and myelokathexis; Weaver syndrome; Weill-Marchesani syndrome 1 and 3; Weill-Marchesani-like syndrome; Weissenbacher-Zweymuller syndrome; Werdnig-Hoffmann disease; Charcot-Marie-Tooth disease; Werner syndrome; WFS1-Related Disorders; Wiedemann-Steiner syndrome; Wilson disease; Wolfram-like syndrome, autosomal dominant; Worth disease; Van Buchem disease type 2; Xeroderma pigmentosum, complementation group b, group D, group E, and group G; X-linked agammaglobulinemia; X-linked hereditary motor and sensory neuropathy; X-linked ichthyosis with steryl-sulfatase deficiency; X-linked periventricular heterotopia; Oto-palato-digital syndrome, type I; X-linked severe combined immunodeficiency; Zimmermann-Laband syndrome and Zimmermann-Laband syndrome 2; and Zonular pulverulent cataract 3.

In some aspects, the present disclosure provides uses of any one of the fusion proteins described herein and a guide RNA targeting this base editor to a target C:G base pair in a nucleic acid molecule in the manufacture of a kit for nucleic acid editing, wherein the nucleic acid editing comprises contacting the nucleic acid molecule with the base editor and guide RNA under conditions suitable for the substitution of the cytosine (C) of the C:G nucleobase pair with an guanine (G). In some embodiments of these uses, the nucleic acid molecule is a double-stranded DNA molecule. In some embodiments, the step of contacting induces separation of the double-stranded DNA at a target region. In some embodiments, the step of contacting thereby comprises the nicking of one strand of the double-stranded DNA, wherein the one strand comprises an unmutated strand that comprises the G of the target C:G nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in an experimental animal, such as a rodent or monkey. In some embodiments, the step of contacting is performed in a cell, such as a human or non-human animal cell.

The present disclosure also provides uses of any one of the fusion proteins described herein as a medicament. The present disclosure also provides uses of any one of the complexes of fusion proteins and guide RNAs described herein as a medicament.

### Base Editor Efficiency

Some aspects of the disclosure are based on the recognition that any of the fusion proteins provided herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate fusion proteins that efficiently modify (e.g. mutate or deaminate) a specific nucleotide within a nucleic acid, without generating a large number of insertions or deletions (i.e., indels) in the nucleic acid. In certain embodiments, any of the fusion proteins provided herein are capable of generating a greater proportion of intended modifications (e.g., C-to-G editing) versus indels. In some embodiments, the fusion proteins provided herein are capable of generating a ratio of intended point mutations to indels that is greater than 1:1. In some embodiments, the fusion proteins provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method, for example the methods used in the below Examples. In some embodiments, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the fusion proteins provided herein are capable of limiting formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the fusion proteins provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation, such as a point mutation, in a nucleic acid (e.g. a nucleic acid within a genome of a subject) without generating a significant number of unintended mutations, such as unintended point mutations. In some embodiments, an intended mutation is a mutation that is generated by a specific base editor bound to a gRNA, specifically designed to generate the intended mutation. In some embodiments, the intended mutation is a mutation associated with a disease or disorder. In some embodiments, the intended mutation is a cytosine (C) to guanine (G) point mutation associated with a disease or disorder. In some embodiments, the intended mutation is a guanine (G) to cytosine (C) point mutation associated with a disease or disorder. In some embodiments, the intended mutation is a cytosine (C) to guanine (G) point mutation within the coding region of a gene. In some embodiments, the intended mutation is a Guanine (G) to cytosine (C) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described in the “Base Editor Efficiency” section, herein, may be applied to any of the fusion proteins, or methods of using the fusion proteins provided herein.

### Methods for Editing Nucleic Acids

Some aspects of the disclosure provide methods for editing a nucleic acid. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to a cytidine deaminase and a uracil binding protein) and a guide nucleic acid (e.g., gRNA), wherein the target region comprises a targeted nucleobase pair, b) inducing strand separation of said target region, c) converting a first nucleobase of said target nucleobase pair in a single strand of the target region to a second nucleobase, d) excising the second nucleobase, thereby creating an abasic site, and e) replacing a third nucleobase complementary to the first nucleobase base with a fourth nucleobase that is a cytosine (C). In some embodiments, the method results in less than 20% indel formation in the nucleic acid. It should be appreciated that in some embodiments, step b is omitted. In some embodiments, the first nucleobase is a cytosine (C). In some embodiments, the second nucleobase is a deaminated cytosine, or uracil. In some embodiments, the third nucleobase is a guanine (G). In some embodiments, the fourth nucleobase is a cytosine (C). In some embodiments, a fifth nucleobase is ligated into the abasic site generated in step (d). In some embodiments the fifth nucleobase is guanine (G). In some embodiments, the method results in less than 19%, 18%, 16%, 14%, 12%, 10%, 8%, 6%, 4%, 2%, 1%, 0.5%, 0.2%, or less than 0.1% indel formation. In some embodiments, at least 5% of the intended base pairs are edited. In some embodiments, at least 10%, 15%, 20%, 25%, 30%, 35%, 40%, 45%, or 50% of the intended base pairs are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand (nicked strand) is hybridized to the guide nucleic acid. In some embodiments, the nicked single strand is opposite to the strand comprising the first nucleobase. In some embodiments, the base editor comprises a Cas9 domain. In some embodiments, the base editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the fusion protein comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors provided herein. In some embodiments, a target window is a deamination window.

In some embodiments, the disclosure provides methods for editing a nucleotide. In some embodiments, the disclosure provides a method for editing a nucleobase pair of a double-stranded DNA sequence. In some embodiments, the method comprises a) contacting a target region of the double-stranded DNA sequence with a complex comprising a base editor and a guide nucleic acid (e.g., gRNA), where the target region comprises a target nucleobase pair, b) inducing strand separation of said target region, c) converting a first nucleobase of said target nucleobase pair in a single strand of the target region to a second nucleobase, d) excising the second nucleobase, thereby creating an abasic site, and e) replacing a third nucleobase complementary to the first nucleobase base with a fourth nucleobase that is a cytosine (C), thereby generating an intended edited base pair, wherein the efficiency of generating the intended edited base pair is at least 5%. It should be appreciated that in some embodiments, step b is omitted. In some embodiments, at least 5% of the intended base pairs are edited. In some embodiments, at least 10%, 15%, 20%, 25%, 30%, 35%, 40%, 45%, or 50% of the intended base pairs are edited. In some embodiments, the method causes less than 19%, 18%, 16%, 14%, 12%, 10%, 8%, 6%, 4%, 2%, 1%, 0.5%, 0.2%, or less than 0.1% indel formation. In some embodiments, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the nicked single strand is hybridized to the guide nucleic acid. In some embodiments, the fusion protein comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the linker is 1-25 amino acids in length. In some embodiments, the linker is 5-20 amino acids in length. In some embodiments, the linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair occurs within the target window. In some embodiments, the target window comprises the intended edited base pair.

### Reduced Off-Target DNA Editing Effects

In some aspects, provided herein are base editors and methods of editing DNA by contacting DNA with any of these disclosed base editors that generate (or cause) reduced off-target effects. In various embodiments, methods are designed for determining the off-target editing frequencies of napDNAbp domain-independent (e.g., Cas9-independent), or napDNAbp domain-dependent (e.g., Cas9-dependent), off-target editing events. Editing events may comprise deamination events and excision events mediated by any of the disclosed CGBEs. Off-target deamination events that are dependent on the napDNAbp-guide RNA complex tend to be in sequences that have high sequence identity (e.g., greater than 60% sequence identity) to the target sequence. These types of events arise because of imperfect hybridization of the napDNAbp-guide RNA complex to sequences that share identity with the target sequence. In contrast, off-target events that occur independently of the napDNAbp-guide RNA complex arise as a result of stochastic binding of the base editor to DNA sequences (often sequences that do not share high sequence identity with the target sequence) due to an intrinsic affinity of the base editor of the nucleotide modification domain (e.g., the deaminase domain) of the base editor with DNA. NapDNAbp-independent (e.g., Cas9-independent) editing events arise in particular when the base editor is overexpressed in the system under evaluation, such as a cell or a subject.

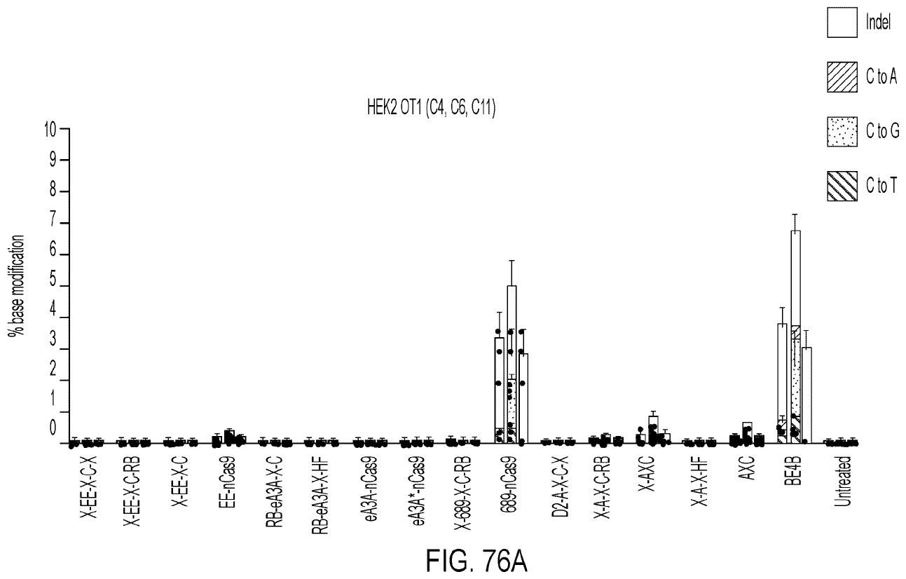

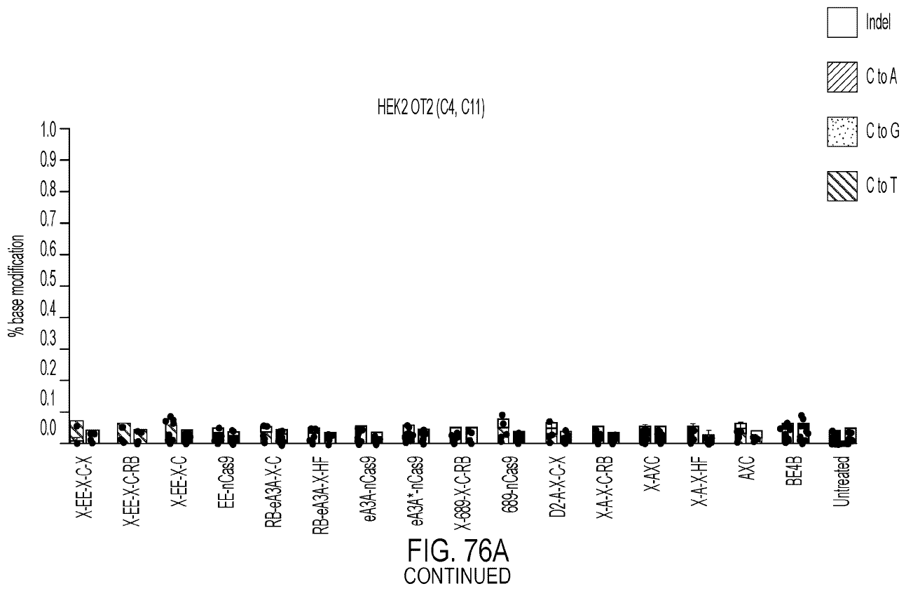

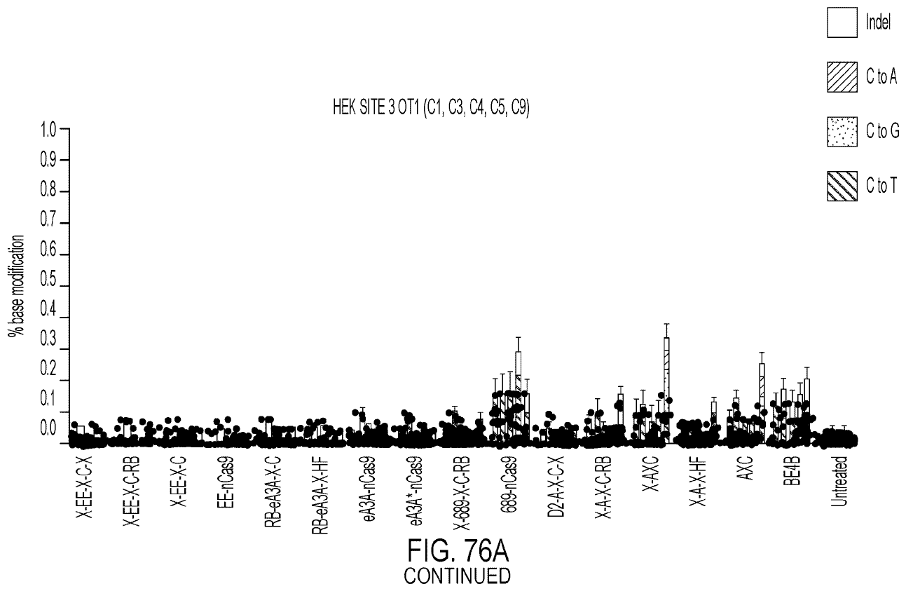

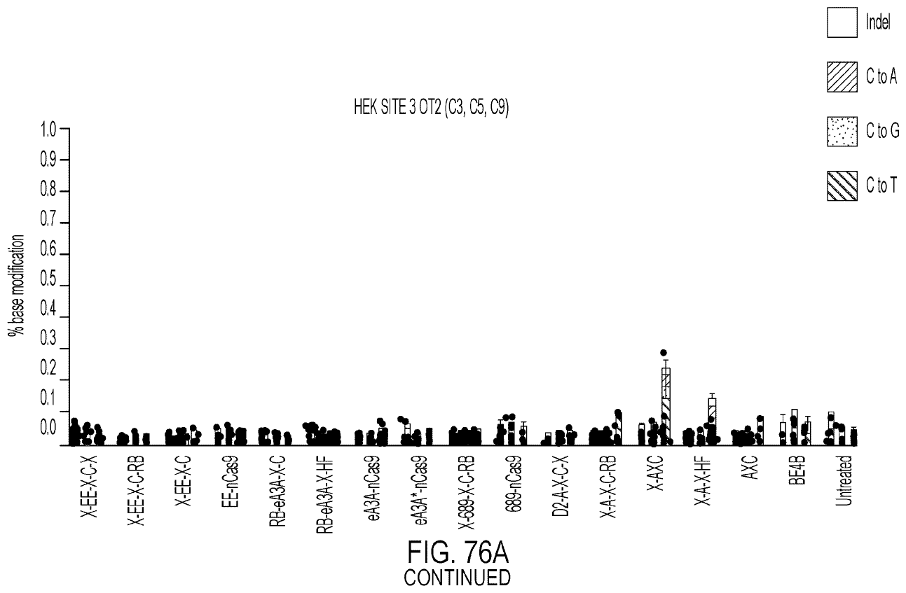

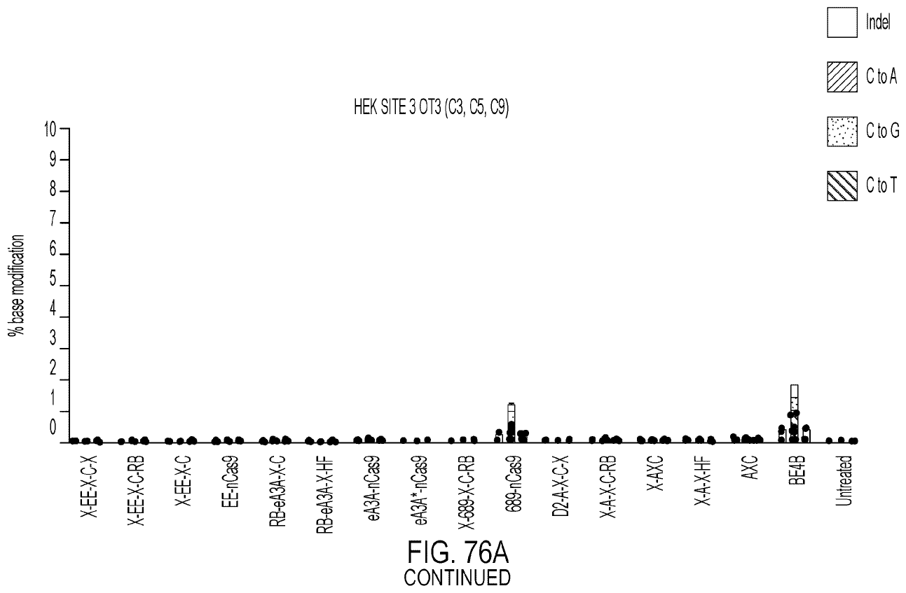

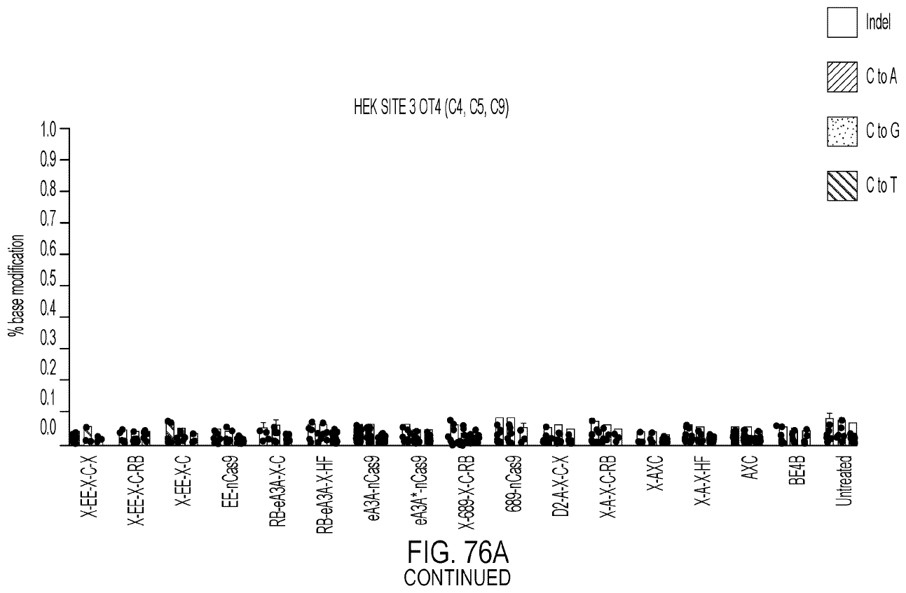

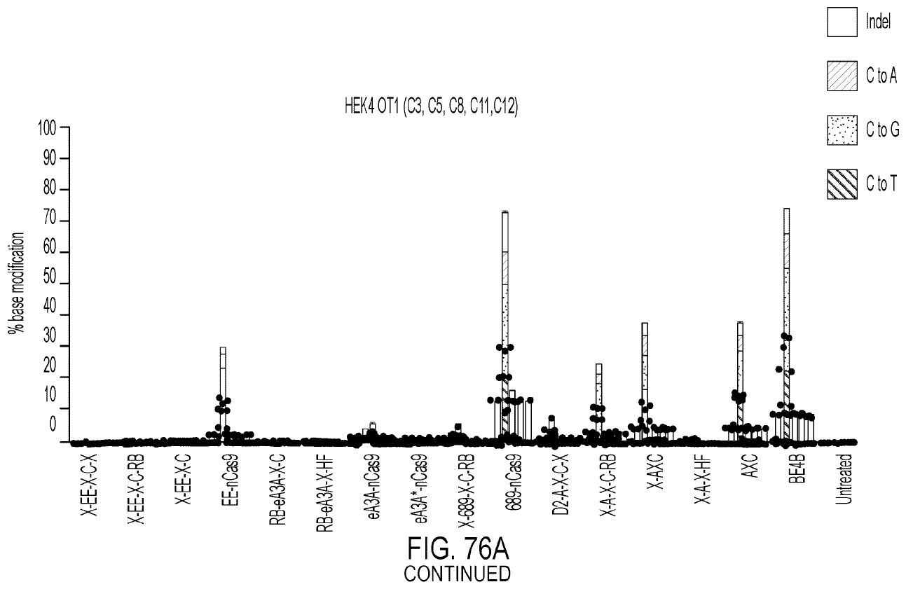

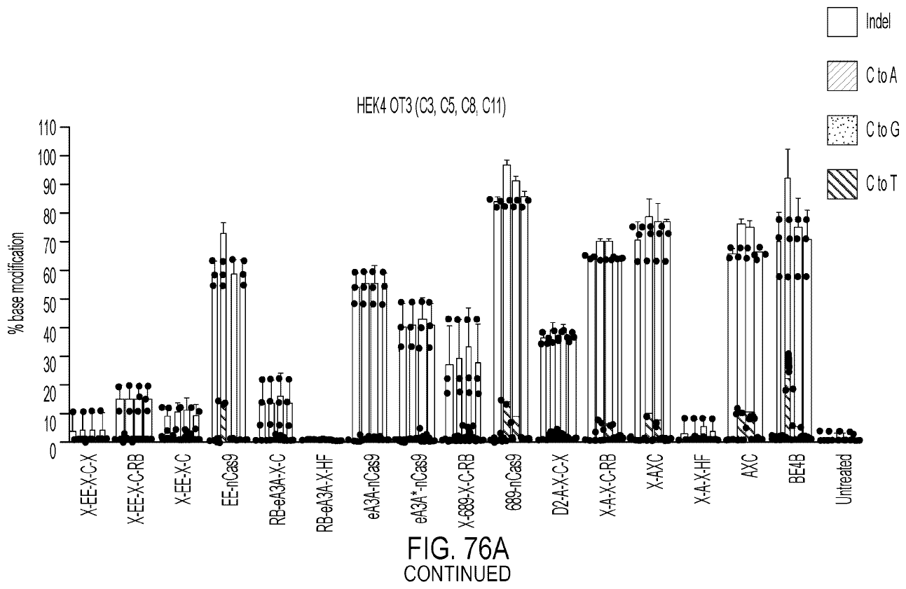

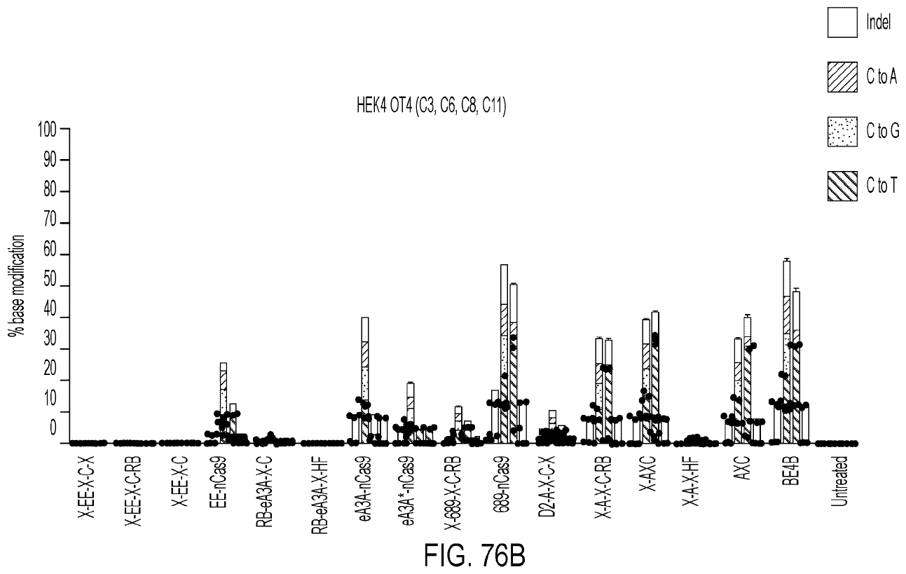

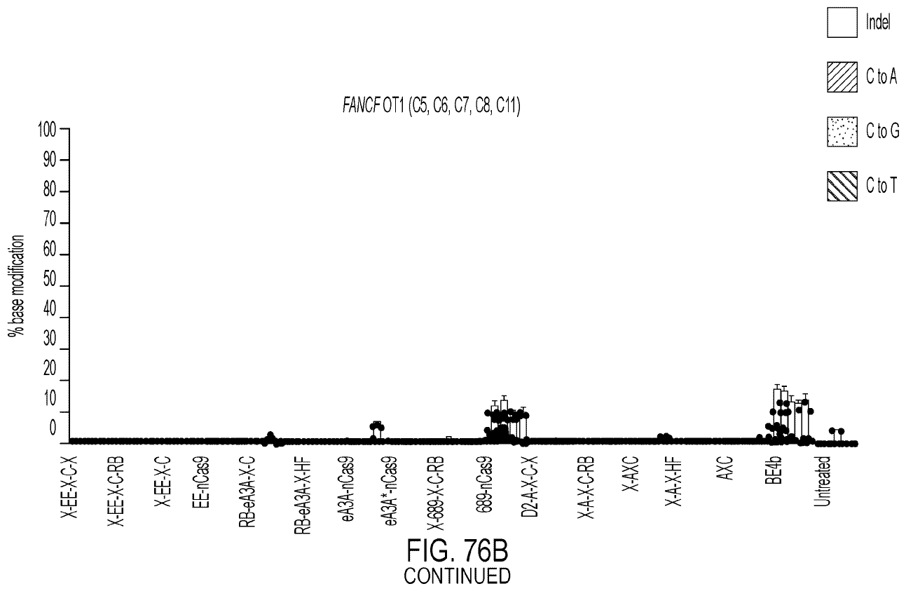

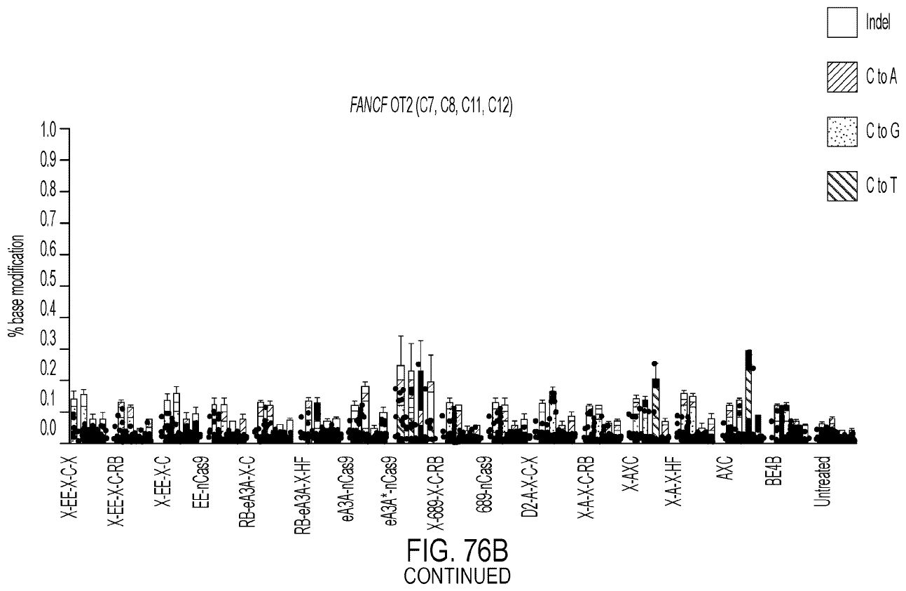

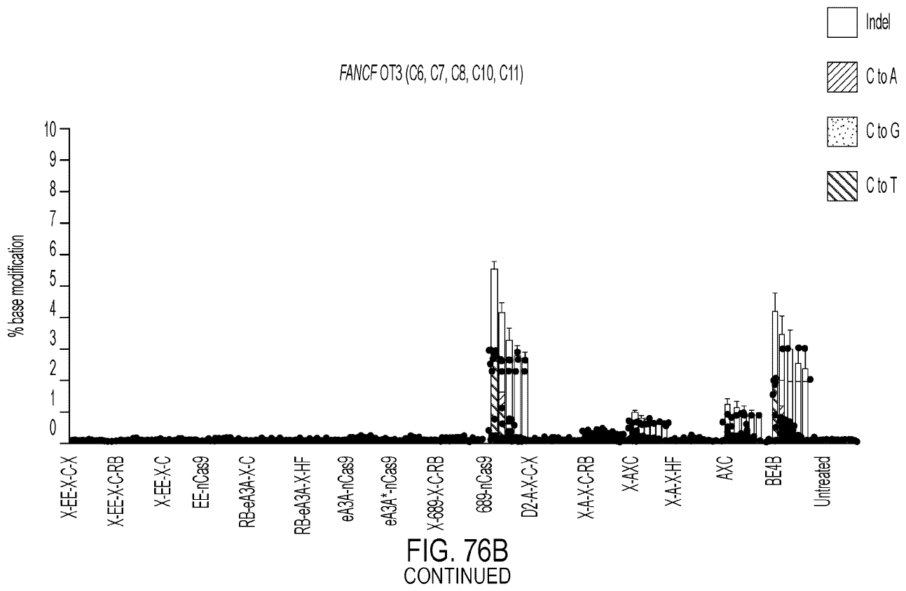

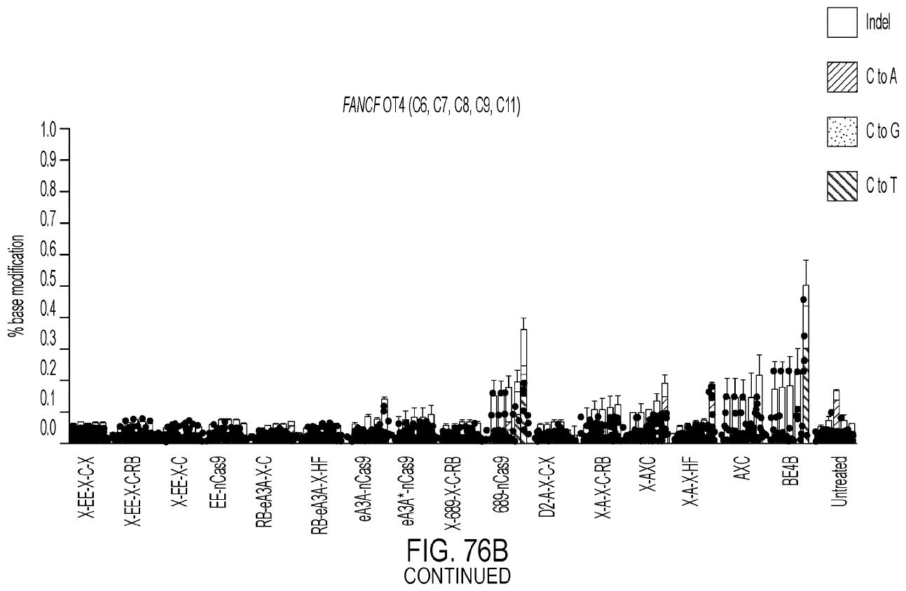

Guide RNA-dependent off-target base editing has been reduced through strategies including installation of mutations that increase DNA specificity into the Cas9 component of base editors, adding 5′ guanosine nucleotides to the sgRNA, or delivery of the base editor as a ribonucleoprotein complex (RNP). Guide RNA-independent off-target editing can arise from binding of the deaminase domain of a base editor to C or A bases in a Cas9-independent manner. The off-target effects of the disclosed base editors may be measured using assays and methods disclosed in and International Application No. PCT/US2020/624628, filed Nov. 25, 2020, incorporated herein by reference. Example 7 below establishes that the disclosed CGBEs exhibit reduced off-target editing relative to their counterpart simple deaminase-nCas9 fusions (i.e., their counterpart cytosine base editors, which lacks any uracil binding proteins). For instance, the RBMX-eA3A-UdgX-HF-nCas9 CGBE exhibited a 52-fold reduced off-target editing relative to the eA3A-nCas9 CBE (see FIGs. 76A and 76B).

Accordingly, in some embodiments, any of the disclosed base editors exhibit about 3-fold, 4-fold, 4.5-fold, 5-fold, 8-fold, 10-fold, 11-fold, 11.5-fold, 12-fold, 15-fold, 20-fold, 30-fold, 40-fold, 45-fold, 50-fold, 55-fold, or greater than 55-fold reduced average editing frequencies of non-target sequences relative to their counterpart cytosine base editors. In some embodiments, the disclosed base editors have 11.5-fold reduced average editing frequencies of non-target sequences relative to their counterpart cytosine base editors. In some embodiments, any of the disclosed base editors exhibit about 3-fold, 4-fold, 4.5-fold, 5-fold, 8-fold, 10-fold, 11-fold, 11.5-fold, 12-fold, 15-fold, 20-fold, 30-fold, 40-fold, 45-fold, 50-fold, 55-fold, or greater than 55-fold reduced editing at non-target cytosines within the editing window relative to their counterpart cytosine base editors. In some embodiments, any of the disclosed base editors exhibit about 3-fold, 5-fold, 8-fold, 10-fold, 11-fold, 12-fold, 15-fold, 20-fold, 30-fold, 40-fold, 45-fold, 50-fold, or greater than 50-fold reduced average editing frequencies of non-target sequences relative to previously described CGBEs.

The disclosed CGBEs may exhibit low off-target editing frequencies, and in particular low Cas9-dependent off-target editing frequencies, while exhibiting high on-target editing efficiencies, at one or more genomic loci. The disclosed CGBEs may exhibit low to no clinically relevant off-target effects (e.g., unintended point mutations in clinically relevant exons). In some embodiments, the disclosed base editors cause off-target DNA editing (e.g. at non-target cytosines) frequencies of less than 6%, less than 5%, less than 4%, less than 3%, less than 2%, less than 1.25%, less than 1%, less than 0.75%, less than 0.5%, less than 0.4%, less than 0.25%, less than 0.2%, less than 0.15%, or less than 0.1% (see FIGS. 76A and 76B). The disclosed base editors, and methods of editing that comprise the use of any of these base editors, may provide an on-target cytosine editing efficiency of greater than 50% and a frequency of off-target editing of less than 1.5%.

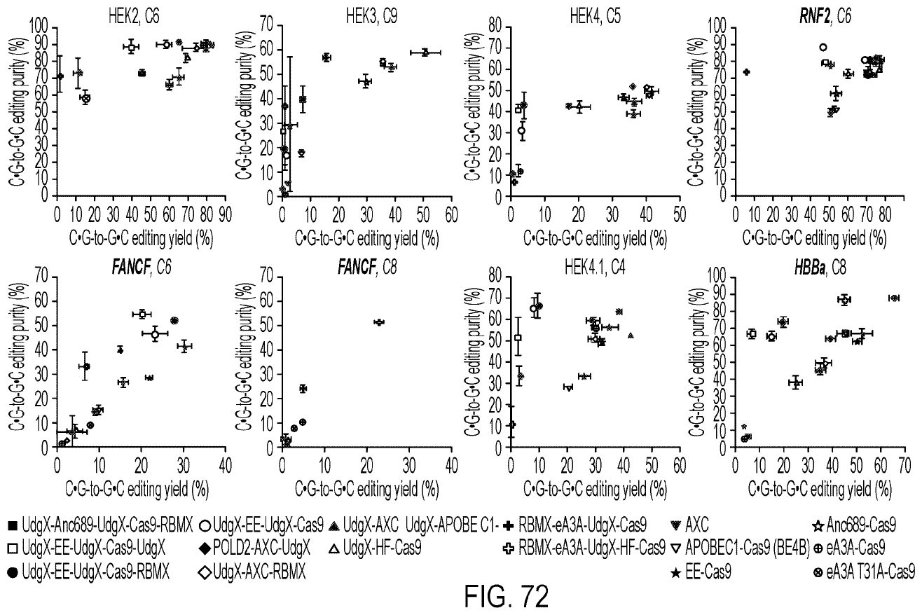

In various embodiments, the disclosed editing methods result in an on-target cytosine base editing efficiency of at least about 50%, 60%, 70%, 73%, 75%, 77%, 80%, 82%, 83%, 84%, 80%, 85%, 86%, 88%, 90%, 95%, 98%, or 99% at the target nucleobase pair. The step of contacting may result in in an efficiency of conversion of the C to a G is at least 70%, 73%, 75%, 77%, 80%, 82%, 83%, 84%, 86%, 88%, 90%, 92.5%, 95%, or 98% (see FIG. 72). In particular, the step of contacting may result in on-target base editing efficiencies of greater than 90%.

In various embodiments, the disclosed editing methods result in a product purity of conversion of the C to a G of at least about 65%, 70%, 73%, 75%, 77%, 80%, 82%, 83%, 84%, 86%, 88%, 90%, 92.5%, or 95%. In some embodiments, the step of contacting may result in a product purity of at least 83%. In some embodiments, the step of contacting may result in a product purity of at least 73%.

### Pharmaceutical Compositions

Other aspects of the present disclosure relate to pharmaceutical compositions comprising any of the base editors, fusion proteins, or the fusion protein-gRNA complexes described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use.

Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention (e.g., a fusion protein or a base editor) in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a CGBE. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the CGBE on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Delivery Methods

The disclosure also provides methods for delivering an cytosine base editor described herein (e.g., in the form of a base editor as described herein, or a vector or construct encoding same) into a cell. Such methods may involve transducing (e.g., via transfection) cells with a plurality of complexes each comprising a base editor and a gRNA molecule. In some embodiments, the gRNA is bound to the napDNAbp domain (e.g., nCas9 domain) of the base editor. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a target sequence. In certain embodiments, the methods involve the transfection of nucleic acid constructs (e.g., plasmids and mRNA constructs) that each (or together) encode the components of a complex of base editor and gRNA molecule. In certain embodiments, any of the disclosed base editors and a gRNA are administered as a protein:RNA complex, such as a ribonucleoprotein complex. In some embodiments, any of the disclosed base editors are administered as an mRNA construct, along with the gRNA molecule. In particular embodiments, administration to cells is achieved by electroporation or lipofection.

In certain embodiments of the disclosed methods, a nucleic acid construct (e.g., an mRNA construct) that encodes the base editor is transfected into the cell separately from the construct that encodes the gRNA molecule. In certain embodiments, these components are encoded on a single construct and transfected together. In other embodiments, the methods disclosed herein involve the introduction into cells of a complex comprising a base editor and gRNA molecule that has been expressed and cloned outside of these cells.

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell.

In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

In another aspect, the disclosure discloses a pharmaceutical composition comprising any one of the presently disclosed vectors. In certain embodiments, the pharmaceutical composition further comprises a pharmaceutically acceptable excipient. In certain embodiments, the pharmaceutical composition further comprises a lipid and/or polymer. In certain embodiments, the lipid and/or polymer is cationic. The preparation of such lipid particles is well known. See, e.g. U.S. Pat. Nos. 4,880,635; 4,906,477; 4,911,928; 4,917,951; 4,920,016; 4,921,757; and 9,737,604, each of which is incorporated herein by reference.

Exemplary methods of delivery of nucleic acids include lipofection, nucleofection, electoporation (e.g., MaxCyte electroporation), stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g. in vitro or ex vivo administration) or target tissues (e.g. in vivo administration). Delivery may be achieved through the use of RNP complexes.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In other embodiments, the method of delivery and vector provided herein is an RNP complex. RNP delivery of base editors markedly increases the DNA specificity of base editing. RNP delivery of base editors leads to decoupling of on- and off-target DNA editing. RNP delivery ablates off-target editing at non-repetitive sites while maintaining on-target editing comparable to plasmid delivery, and greatly reduces off-target DNA editing even at the highly repetitive VEGFA site 2. See Rees, H. A. et al., Improving the DNA specificity and applicability of base editing through protein engineering and protein delivery, Nat. Commun. 8, 15790 (2017), U.S. Pat. No. 9,526,784, issued Dec. 27, 2016, and U.S. Pat. No. 9,737,604, issued Aug. 22, 2017, each of which is incorporated by reference herein.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and Y² cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. Reference is made to US 2003/0087817, published May 8, 2003, International Patent Application No. WO 2016/205764, published Dec. 22, 2016, International Patent Application No. WO 2018/071868, published Apr. 19, 2018, U.S. Patent Publication No. 2018/0127780, published May 10, 2018, and International Publication No. WO2020/236982, published Nov. 26, 2020, the disclosures of each of which are incorporated herein by reference.

In various embodiments, the base editor constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editor that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VPlu, which has the genome of AAV2, capsid backbone of AAV5 and VPlu of AAV1. Other non-limiting example of derivatives and pseudotypes that have chimeric VP1 proteins are rAAV2/5-8VPlu, rAAV2/9-1VPlu, and rAAV2/9-8VPlu.

AAV derivatives/pseudotypes, and methods of producing such derivatives/pseudotypes are known in the art (see, e.g., Mol. Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

In some embodiments, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning CGBE.

These split intein-based methods overcome several barriers to in vivo delivery. For example, the DNA encoding base editors is larger than the recombinant AAV (rAAV) packaging limit, and so requires different solutions. One such solution is formulating the editor fused to split intein pairs that are packaged into two separate rAAV particles that, when co-delivered to a cell, reconstitute the functional editor protein. Several other special considerations to account for the unique features of base editing are described, including the optimization of second-site nicking targets and properly packaging base editors into virus vectors, including lentiviruses and rAAV.

Accordingly, the disclosure provides dual rAAV vectors and dual rAAV vector particles that comprise expression constructs that encode two halves of any of the disclosed base editors, wherein the encoded base editor is divided between the two halves at a split site. In some embodiments, the two halves may be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning CGBE.

In various embodiments, the base editors may be engineered as two half proteins (i.e., an CGBE N-terminal half and a CGBE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

Additional methods for the delivery of nucleic acids to cells are known to those skilled in the art. See, for example, US Pub. No. 2003/0087817, incorporated herein by reference.

It should be appreciated that any base editor, e.g., any of the base editors provided herein, may be introduced into the cell in any suitable way, either stably or transiently. In some embodiments, a base editor may be transfected into the cell. In some embodiments, the cell may be transduced or transfected with a nucleic acid construct that encodes a base editor. For example, a cell may be transduced (e.g., with a virus encoding a base editor), or transfected (e.g., with a plasmid encoding a base editor) with a nucleic acid that encodes a base editor, or the translated base editor. Such transduction may be a stable or transient transduction. In some embodiments, cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into cells through electroporation, transient (e.g., lipofection) and stable genome integration (e.g., piggybac) and viral transduction or other methods known to those of skill in the art.

### Kits and Cells

Some aspects of this disclosure provide kits comprising a nucleic acid construct comprising a nucleotide sequence encoding a cytosine deaminase capable of deaminating an adenosine in a deoxyribonucleic acid (DNA) molecule. In some embodiments, the nucleotide sequence encodes any of the cytosine deaminases provided herein. In some embodiments, the nucleotide sequence comprises a heterologous promoter that drives expression of the cytosine deaminase. The nucleotide sequence may further comprise a heterologous promoter that drives expression of the gRNA, or a heterologous promoter that drives expression of the base editor and the gRNA.

In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone.

The disclosure further provides kits comprising a nucleic acid construct, comprising (a) a nucleotide sequence encoding a napDNAbp (e.g., a Cas9 domain) fused to a cytosine deaminase, or a base editor comprising a napDNAbp (e.g., Cas9 domain) and an cytosine deaminase as provided herein; and (b) a heterologous promoter that drives expression of the sequence of (a). In some embodiments, the kit further comprises an expression construct encoding a guide nucleic acid backbone, (e.g., a guide RNA backbone), wherein the construct comprises a cloning site positioned to allow the cloning of a nucleic acid sequence identical or complementary to a target sequence into the guide nucleic acid (e.g., guide RNA backbone).

Some embodiments of this disclosure provide cells comprising any of the base editors or complexes provided herein. In some embodiments, the cells comprise nucleotide constructs that encodes any of the base editors provided herein. In some embodiments, the cells comprise any of the nucleotides or vectors provided herein. In some embodiments, the cell is a stem cell. In some embodiments, the cell is a mouse embryonic stem cell (mESC). In some embodiments, the cell is a human stem cell, such as a human stem and progenitor cell (HSPC).

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. In some embodiments, the cell has been removed from a subject and contacted ex vivo with any of the disclosed base editors, complexes, vectors, or polynucleotides.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO—IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK293, HAP-1, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### EXAMPLES

### Cytosine (C) to Guanine (G) Base Editors Through Abasic Site Generation and Engineered Specific Repair

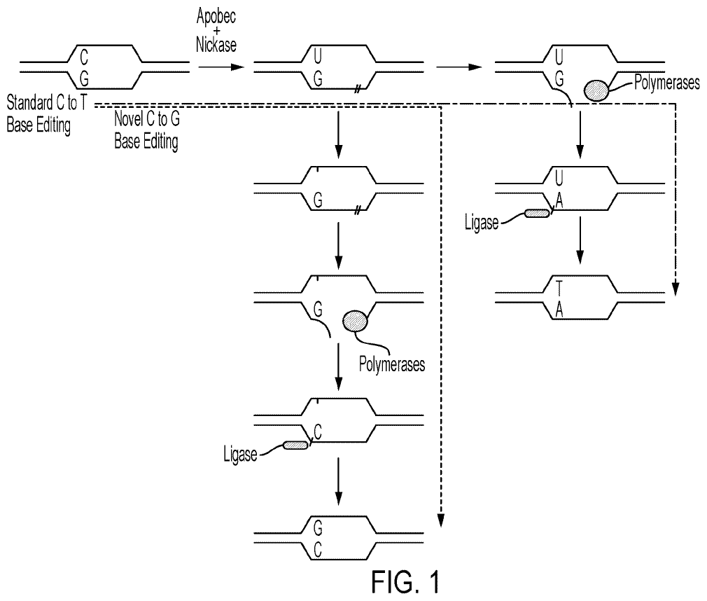

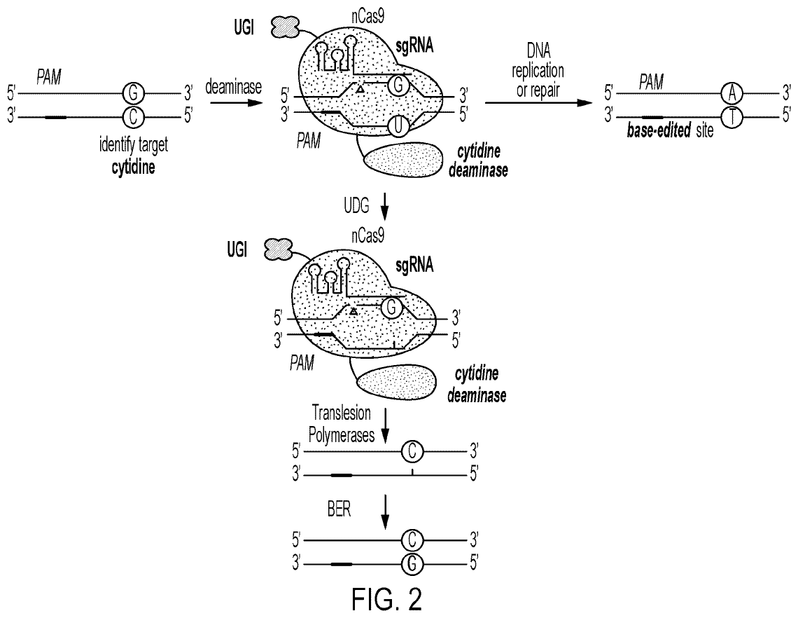

Sequencing data for the HEK2, RNF2, and FANCF sites is given below. Data presented represents base editing values for the most edited C in the window. This is C6 for HEK2, C6 for RNF2, and C6 for FANCF. The sequences for the three different sites before and after base editing are as follows: HEK2: GAACACAAAGCATAGACTGC (SEQ ID NO: 110) (sequencing reads CTTGTGTTTCGTATCTGACG (SEQ ID NO: 111)); RNF2: GTCATCTTAGTCATTACCTG (SEQ ID NO: 112) (sequencing reads CAGTAGAATCAGTAATGGAC (SEQ ID NO: 113)); and FANCF: GGAATCCCTTCTGCAGCACC (SEQ ID NO: 114) (sequencing reads the same). For both HEK2 and RNF2, the non-target strand was sequenced (this strand contains G's complementary to the target C's). For FANCF the target strand was sequenced (this strand contains the target C's). A schematic for C to T base editing (e.g., using BE3, which is a C to T base editor) and C to G base editing is shown in FIGS. 1 and 2. Certain DNA polymerases are known to replace bases opposite abasic sites with G. One strategy to achieve C to G base editing is to induce the creation of the abasic site, then recruit or tether such a polymerase to replace the G opposite the abasic site with a C. This could provide access to all editors, if C and T can be excised and repaired with all the polymerases based on the polymerases' predetermined base preferences.

Different fusion constructs are summarized below and are shown in Table 1. UdgX is an isoform of UDG known to bind tightly to uracil with minimal uracil-excision activity. UdgX* is a mutated version of UdgX (Sang et al. NAR, 2015) that was observed to lack uracil excision activity by an in vitro assay in Sang et al. UdgX_On is another mutated version of UdgX (Sang et al. NAR, 2015) observed to have an increased uracil excision activity in the same in vitro assay reported in Sang et al. UDG is the enzyme responsible for the excision of uracil from DNA to create an abasic site. Rev7 is a component of the Rev1/Rev3/Rev7 complex known to incorporate C opposite an abasic site. RevI is the enzymatic component of the above mentioned complex. Polymerases Alpha, Beta, Gamma, Delta, Epsilon, Gamma, Eta, Iota, Kappa, Lambda, Mu, and Nu are eukaryotic polymerases with different preferences for base incorporation opposite an abasic site.

TABLE 1
Construct Reference Key

| Construct | Definition |
|---|---|
| BE3 | Published base editing construct |
| BE3_UdgX | UGI replaced with Uracil binding protein, UdgX |
| BE3_UdgX* | UGI replaced with UdgX isoform with diminished |
| | binding affinity to Uracil |
| BE3_REV7 | UGI replaced with a component of C-integrating |
| | translesion synthesis machinery |
| BE2_UDG | dCas9 based construct (no nicking) where UGI is |
| | replaced with uracil deglycosylase |
| BE3_UDG | UGI is replaced with uracil deglycosylase (BE3) |
| BE2_UdgX_On | dCas9 construct where UGI is replaced with |
| | UdgX with an activating mutation that |
| | increases Uracil excision |
| BE3_UdgX_On | UGI replaced with UdgX with an activating |
| | mutation that increases Uracil excision |
| SMUG1 | UGI replaced with SMUG1, a ssDNA uracil |
| | deglycosylase |

### Constructs Used in the Examples:

BE3_Full Length—This is a C to T base editor construct comprising a cytidine deaminase, a nCas9, and a uracil glycosylase inhibitor (UGI) domain.

(SEQ ID NO: 115)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIARLYHHA
DPRNRQGLRDLISSGVTIQIMTEQESGYCWRNFVNYSPSNEAHWPRYPHLWVRLYVLELYCII
LGLPPCLNILRRKQPQLTFFTIALQSCHYQRLPPHILWATGLKSGSETPGTSESATPESDKKYSI
GLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKEDYFKKI
ECFDSVEISGVEDRFNASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGDSGGSTNLSDIIEKETG
KQLVIQESILMLPEEVEEVIGNKPESDILVHTAYDESTDENVMLLTSDAPEYKPWALVIQDSN
GENKIKMLSGGSPKKKRKV

BE3_No UGI—This construct is the above BE3 construct, lacking the UGI domain.

(SEQ ID NO: 116)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIARLYHHA
DPRNRQGLRDLISSGVTIQIMTEQESGYCWRNFVNYSPSNEAHWPRYPHLWVRLYVLELYCII
LGLPPCLNILRRKQPQLTFFTIALQSCHYQRLPPHILWATGLKSGSETPGTSESATPESDKKYSI
GLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKEDYFKKI
ECFDSVEISGVEDRFNASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGD

Cas9 Nickase Sequence—Used in BE3.

(SEQ ID NO: 21)
MDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLF
EDREMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAVVGTA
LIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGD

dCas9 Sequence—Used in BE2

(SEQ ID NO: 22)
MDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLF
EDREMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLY
LYYLQNGRDMYVDQELDINRLSDYDVDAIVPQSFLKDDSIDNKVLTRSDKNRGKSDNVPSEE
VVKKMKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAVVGTA
LIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGD

BE3_Replace UGI with UDG, UdgX variants, Polymerases—In the below construct, the NLS sequence is identified by underlining and linkers are identified in italics. The “[UGI]” indicated in the sequence below identifies the location where UDG, UDG variants (e.g., UDG, UdgX* (R107S), and UdgX_On (H109S)), Rev7, and Smug1, were inserted (rather than the UGI of BE3). The “[Polymerase]” indicated in the sequence below identifies the location where polymerases (e.g., Pol Beta, Pol Lambda, Pol Eta, Pol Mu, Pol Iota, Pol Kappa, Pol Alpha, Pol Delta, Pol Gamma, and Pol Nu), and Rev1 were inserted.

(SEQ ID NO: 117)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIARLYHHA
DPRNRQGLRDLISSGVTIQIMTEQESGYCWRNFVNYSPSNEAHWPRYPHLWVRLYVLELYCII
LGLPPCLNILRRKQPQLTFFTIALQSCHYQRLPPHILWATGLKSGSETPGTSESATPESDKKYSI
GLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKEDYFKKI
ECFDSVEISGVEDRFNASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLQNEKLYLYYLQN
GRDMYVDQELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNT
KYDENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSGGS
[UGI]
(SEQ ID NO: 120)
SGGSGGSGGS
[Polymerase]
(SEQ ID NO: 41)
PKKKRKV

N-terminal UDG (insert UDG (Tyr147Ala) or UDG (Asn204Asp))+Cas9 nickase and Polymerase at C-terminus—In the below construct, the NLS sequence is identified by underlining and linkers are identified in italics. The “[UDGvariants]” indicated in the sequence below identifies the location where UDG Tyr147Ala and UDG Asn204Asp, were inserted. The “[Polymerase]” indicated in the sequence below identifies the location where polymerases (e.g., Pol Beta, Pol Lambda, Pol Eta, Pol Mu, Pol Iota, Pol Kappa, Pol Alpha, Pol Delta, Pol Gamma, and Pol Nu), and Rev1 were inserted.

[UDGvariants]
(SEQ ID NO: 118)
SETPGTSESATPESDKKYSIGLAIGTNSVGWAVITDEYKVPSKKFKVL
GNTDRHSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLALAHM
IKFRGHFLIEGDLNPDNSDVDKLFIQLVQTYNQLFEENPINASGVDAKAILSARLSKSRRLENLI
AQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQRTFDNGSIPHQIH
LGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKIIKDK
DFLDNEENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQFYKVRE
INNYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKLKSVKEL
LGITIMERSSFEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIHQSITG
LYETRIDLSQLGGD
(SEQ ID NO: 103)
SGGS
[Polymerase]
(SEQ ID NO: 41)
PKKKRKV

### Example 1: C to G Approach 1—Increase Abasic Site Formation

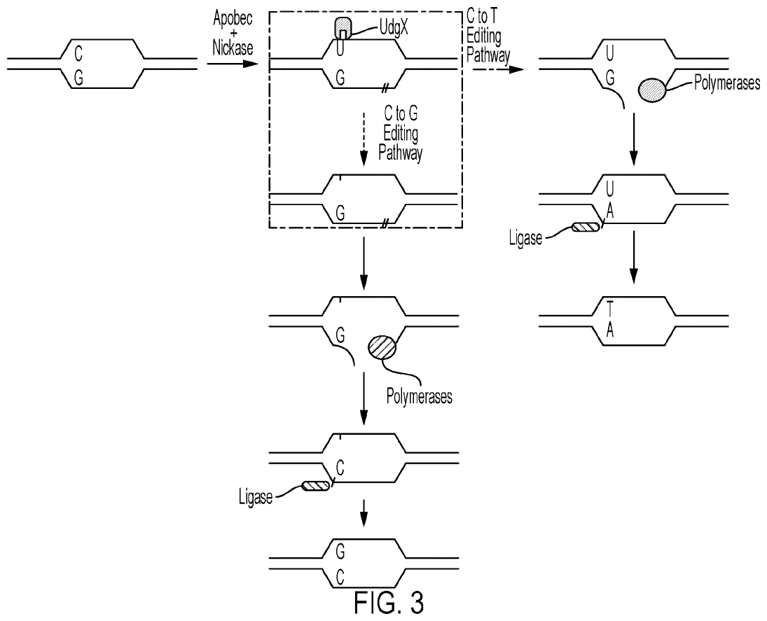

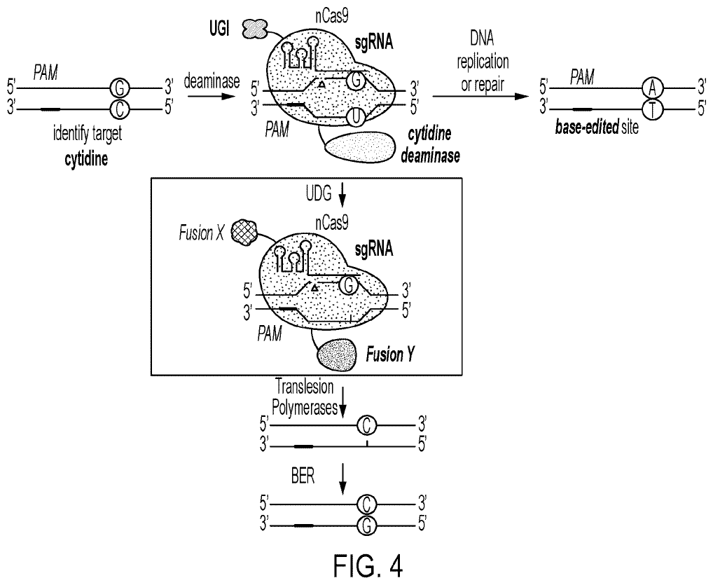

If an abasic site is more efficiently generated, it is expected that the total flux through the C to G base editing pathway will be increased. A schematic representation of base editors used in this approach is shown in FIGS. 3 and 4. Using UdgX, an orthologue of UDG identified to bind tightly to Uracil with minimal uracil excising activity, increases the amount of C to G editing. Without wishing to be bound by any particular theory, UdgX near-covalent binding to U mimics a lesion that instigates translesion polymerase-type repair.

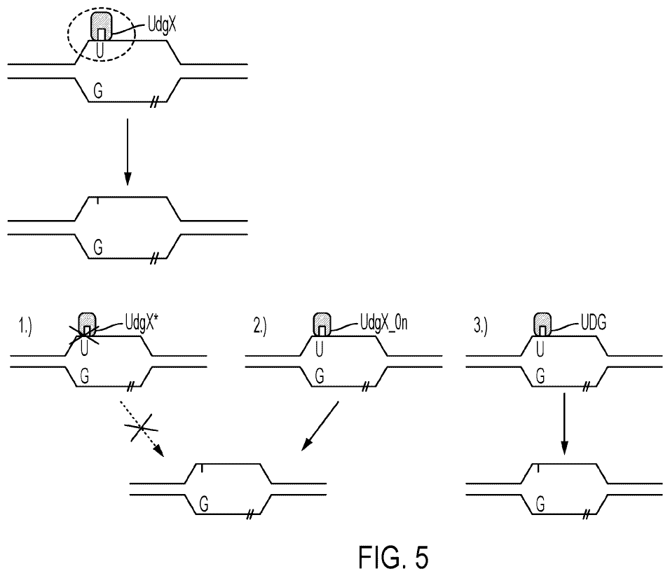

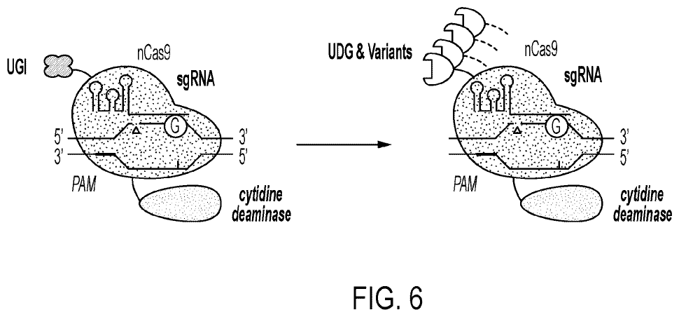

Further, UdgX has a low level catalytic activity which, in combination with tight binding, excises the U and leads to abasic site formation. Abasic site formation allows for off-target products and preferential generation of this lesion leads to more product. This is supported through different experiments and base editors, which are illustrated in FIGS. 5 and 6.

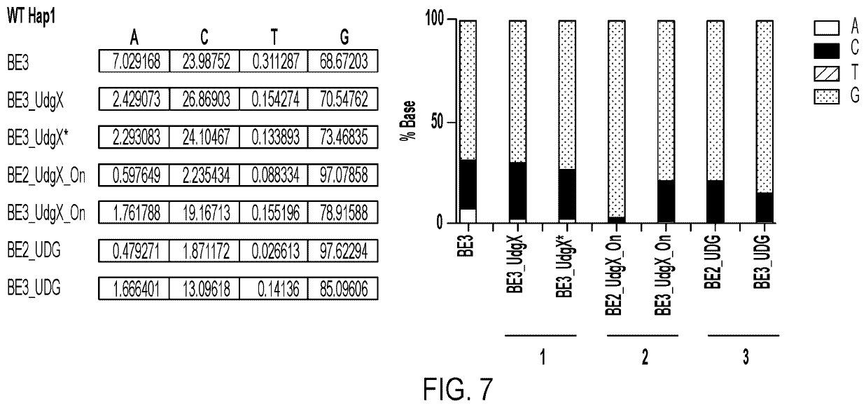

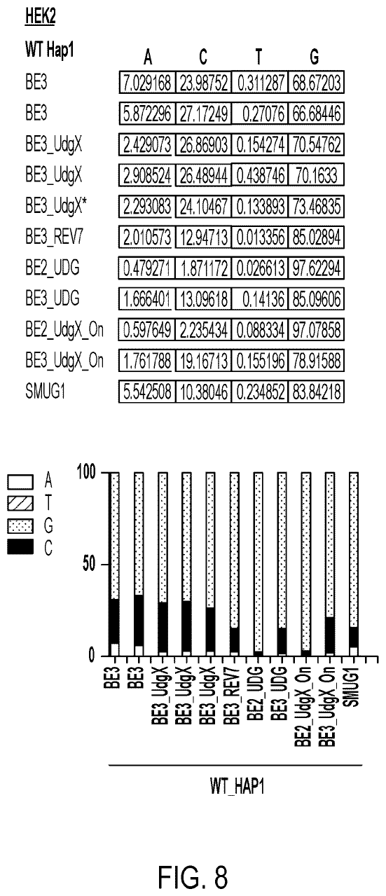

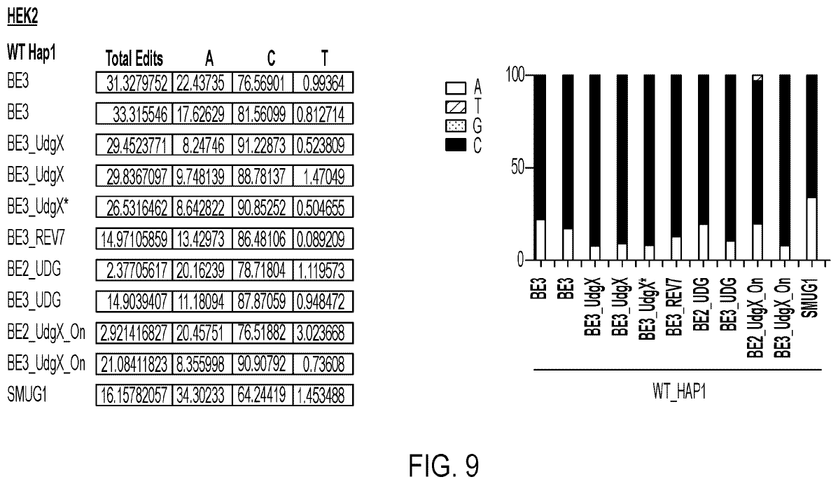

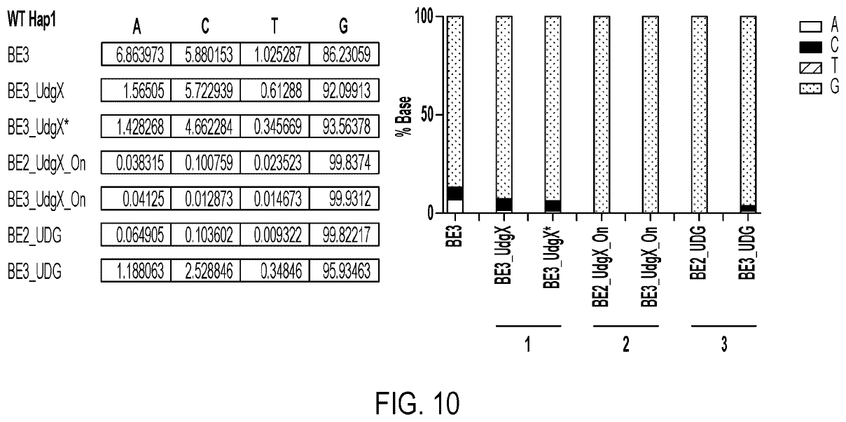

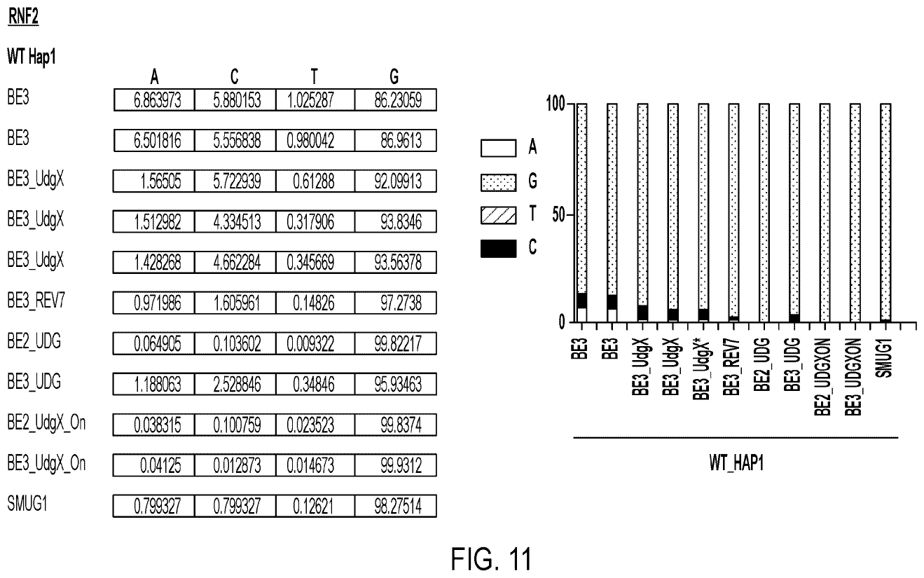

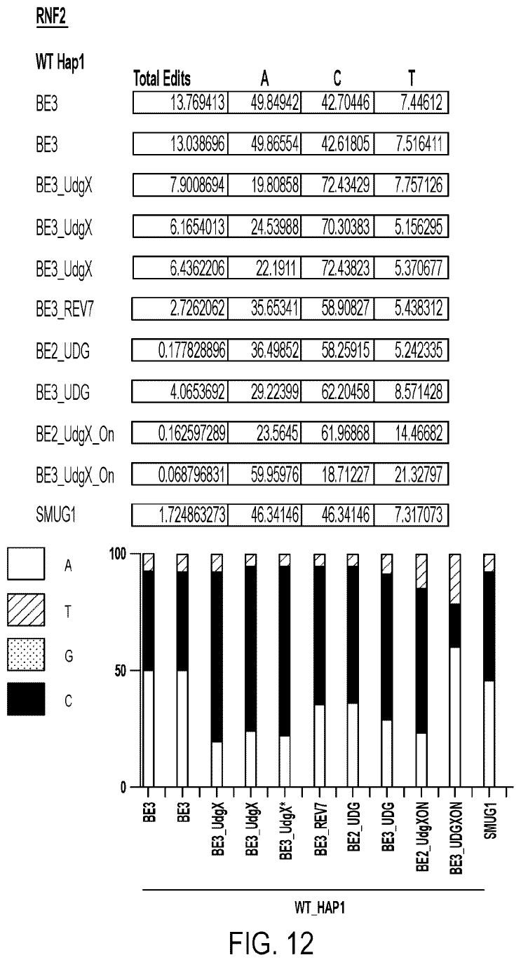

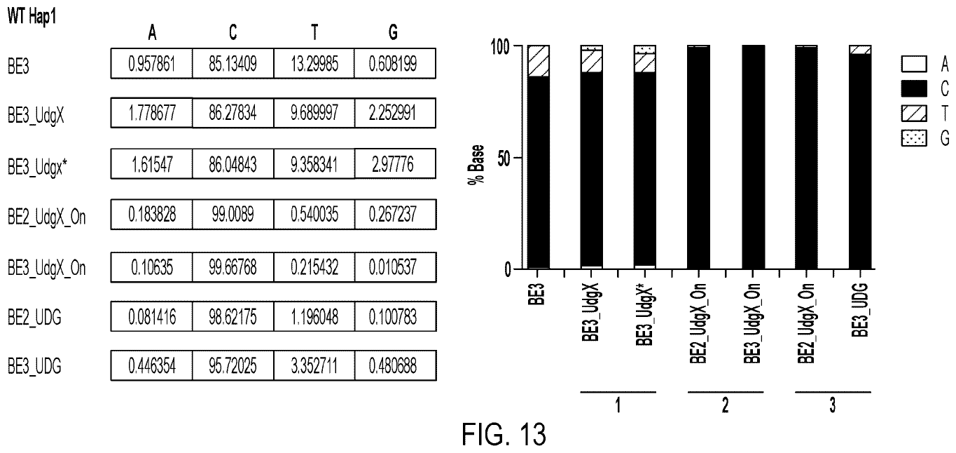

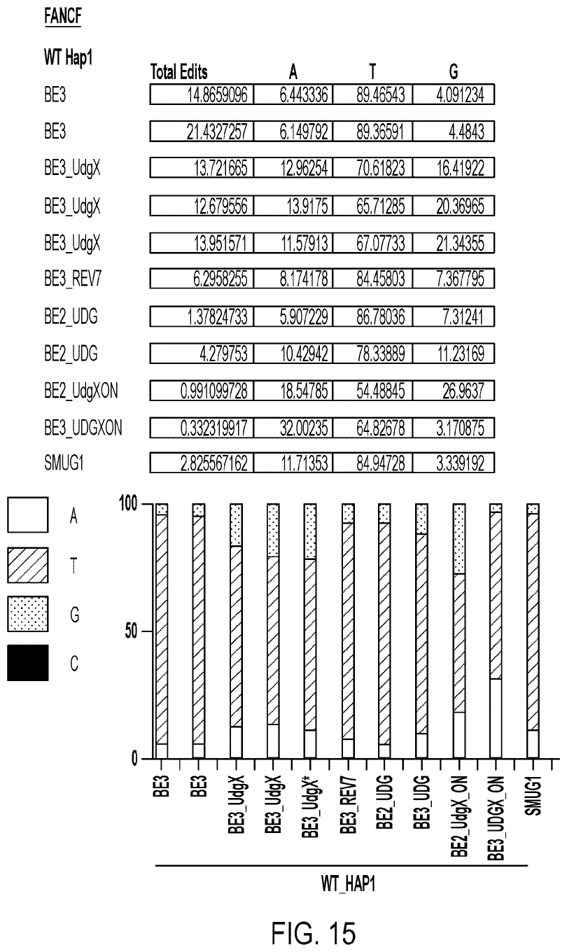

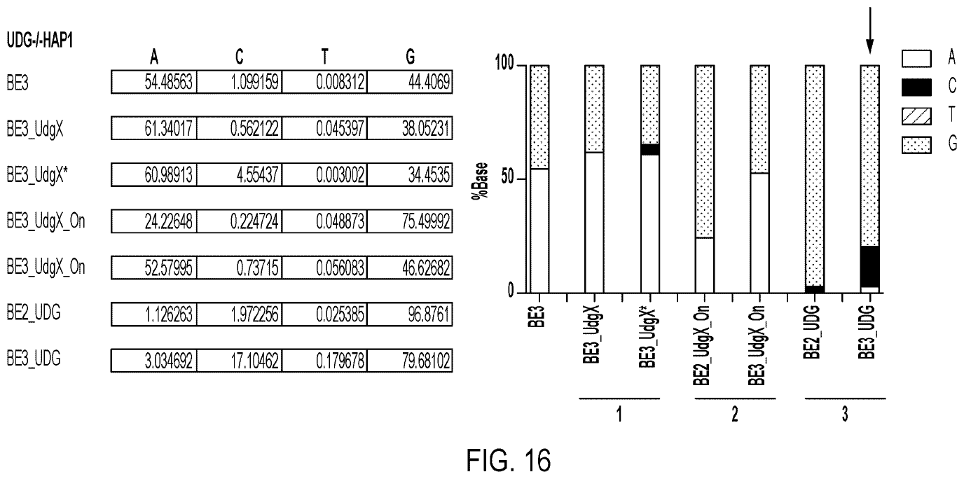

The results of C to G base editing at HEK2, RNF2, and FANCF sites in WT cells using seven base editors (BE3; BE3_UdgX; BE3_UdgX*; BE2_UdgX_On; BE3_UdgX_On; BE2_UDG; and BE3_UDG) are shown in FIGS. 7 through 15. These figures show the results for C to G editing at the most edited position (C6) at the three representative sites that have high, medium, and low tolerance to sequence perturbation from standard C to T editing.

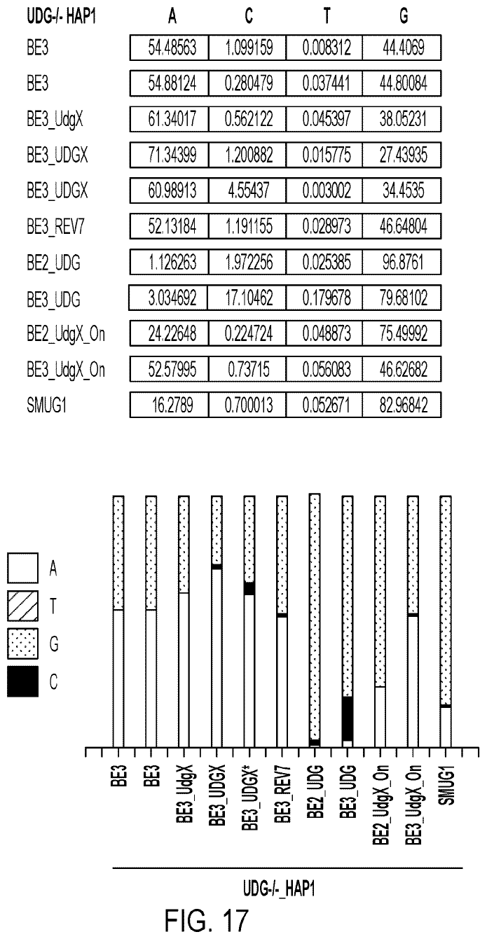

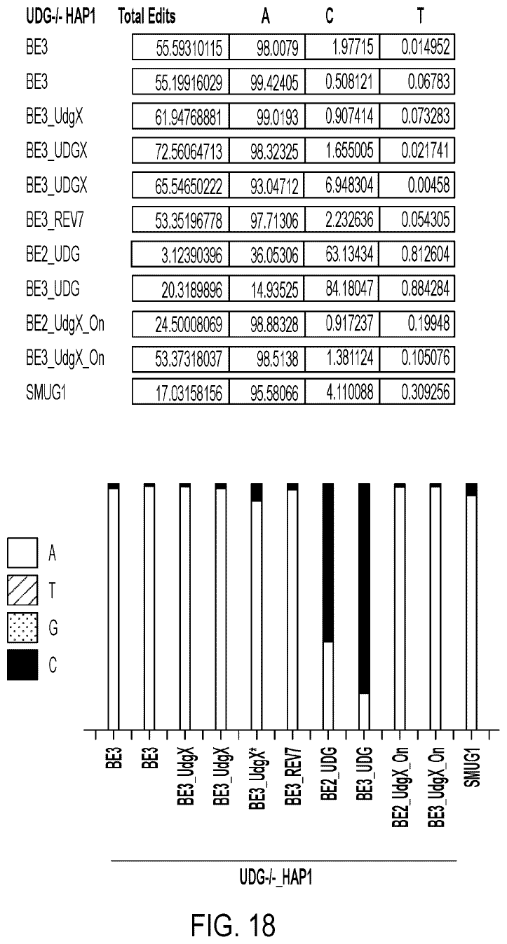

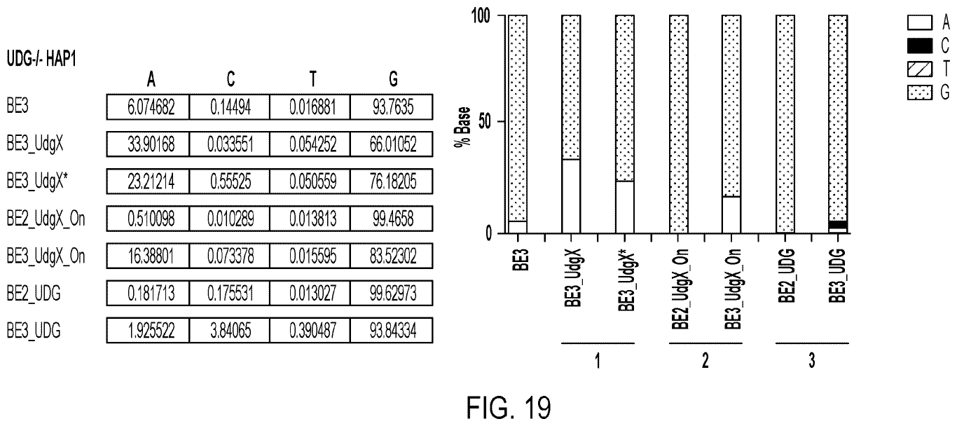

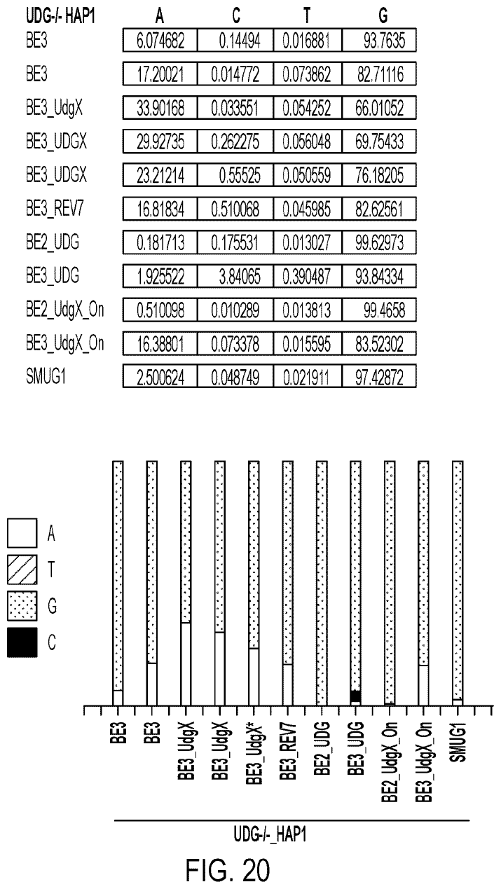

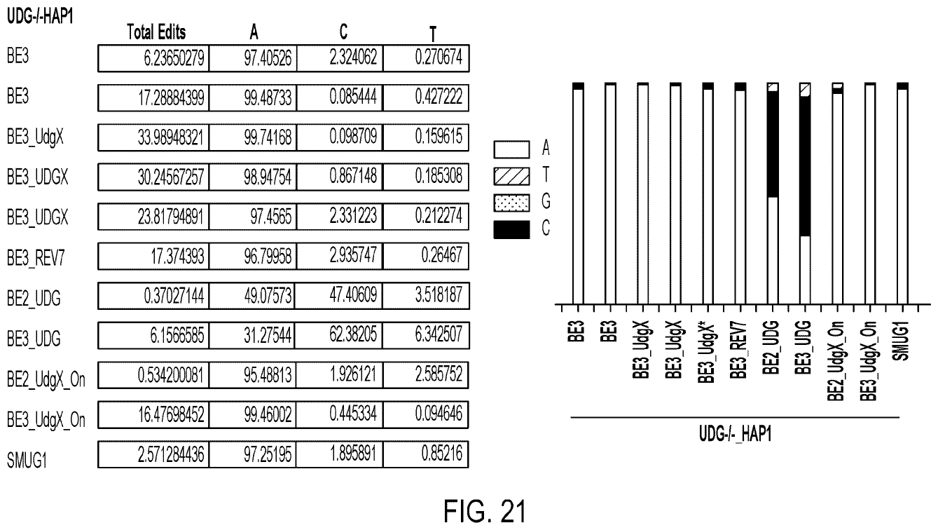

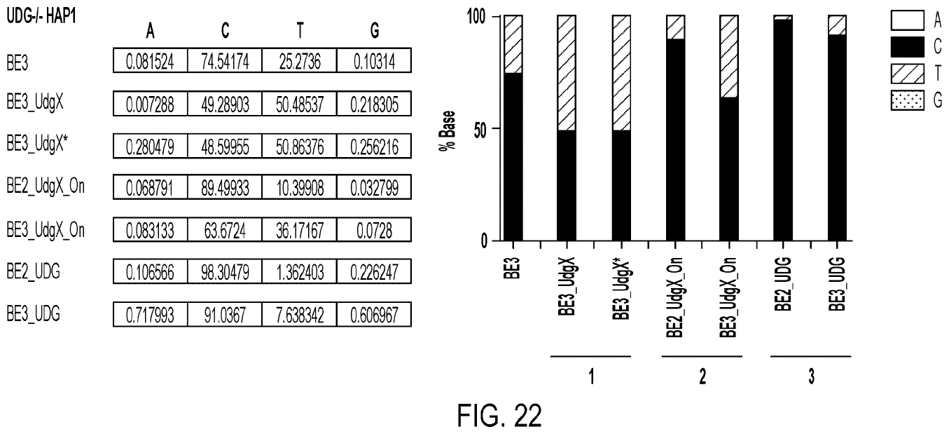

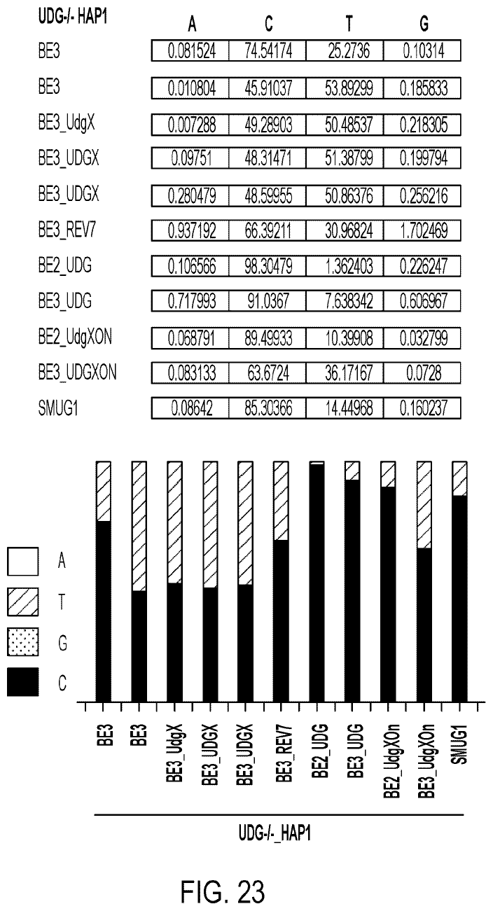

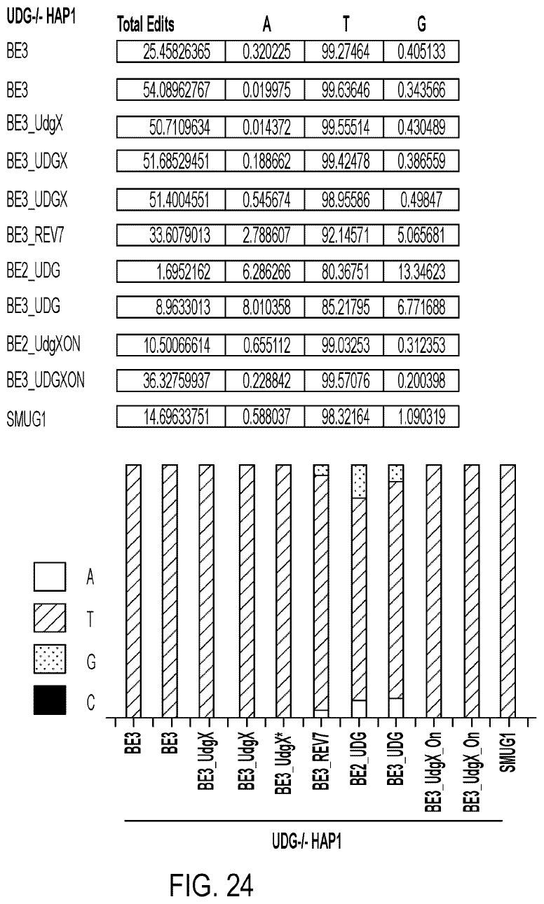

Results of C to G base editing at HEK2, RNF2, and FANCF sites in UDG−/− cells using various C to G base editors (BE3; BE3_UdgX; BE2_UNG; BE3_UNG; BE2UdgX_On; BE3UdgX_On; and SMUG1) are shown in FIGS. 16 through 24.

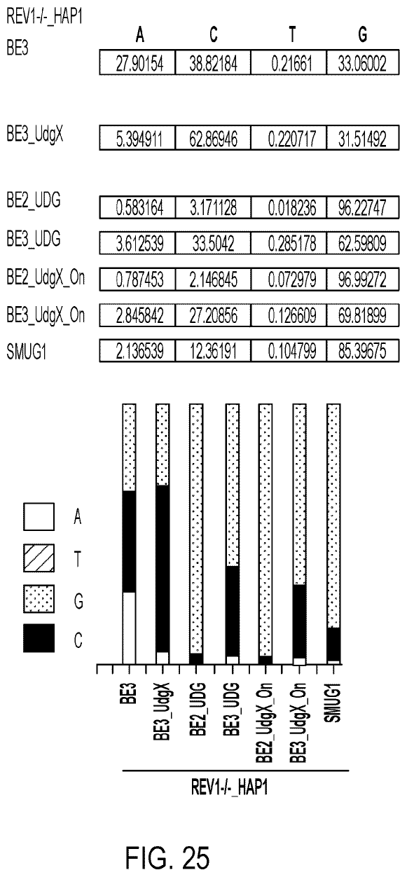

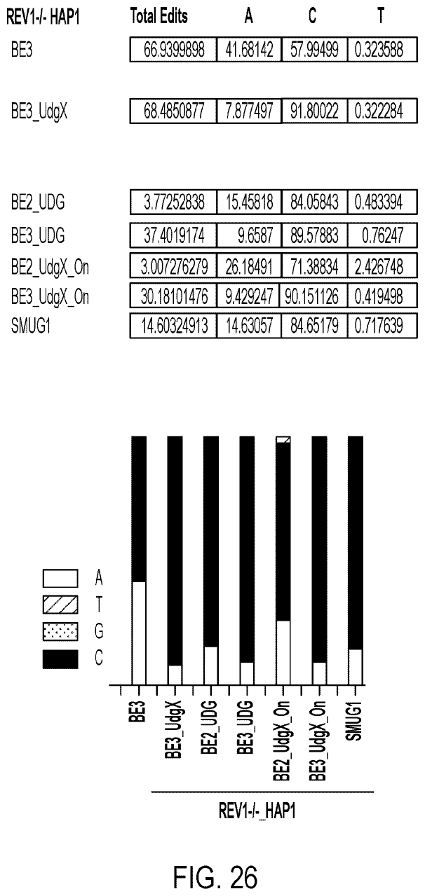

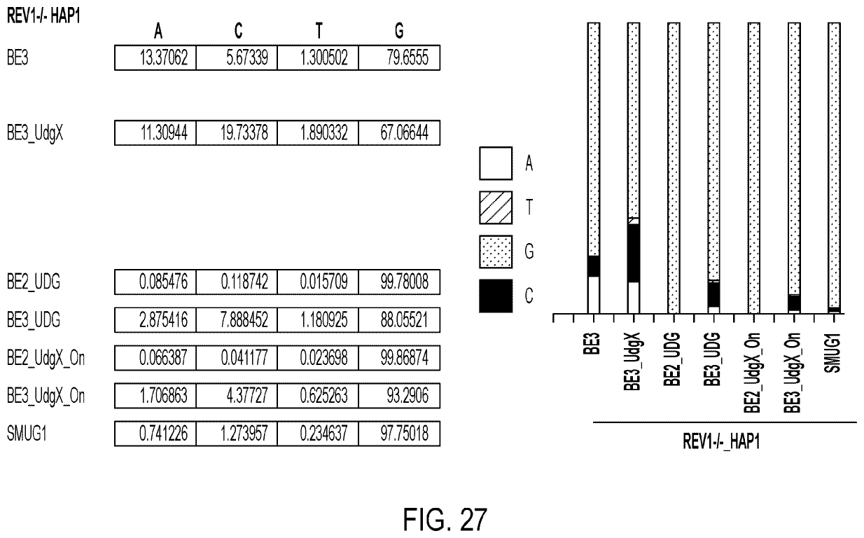

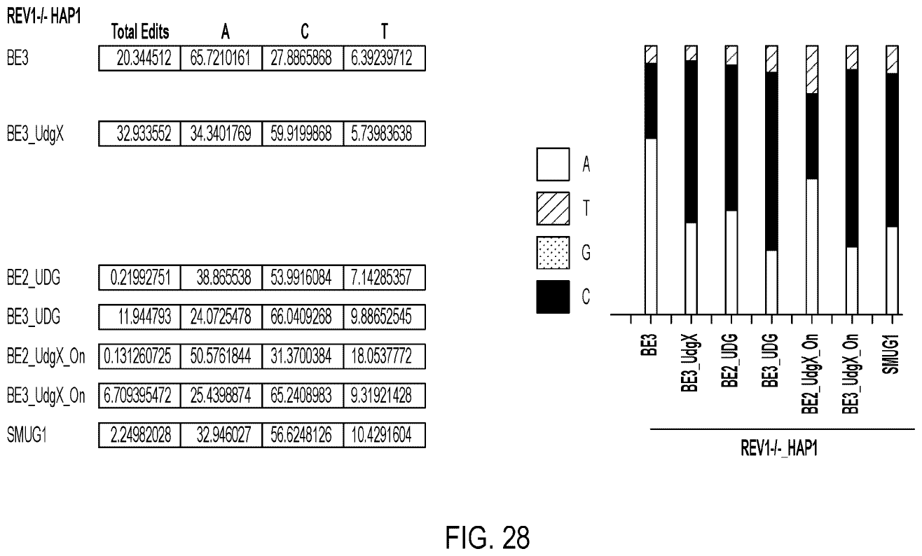

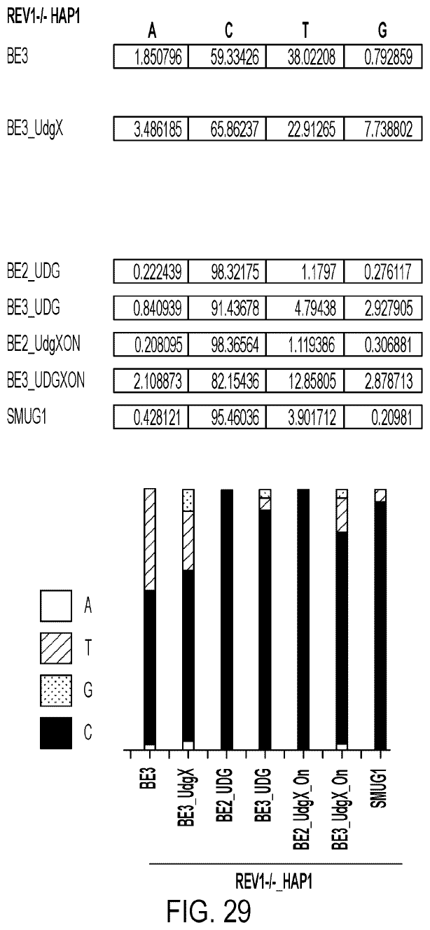

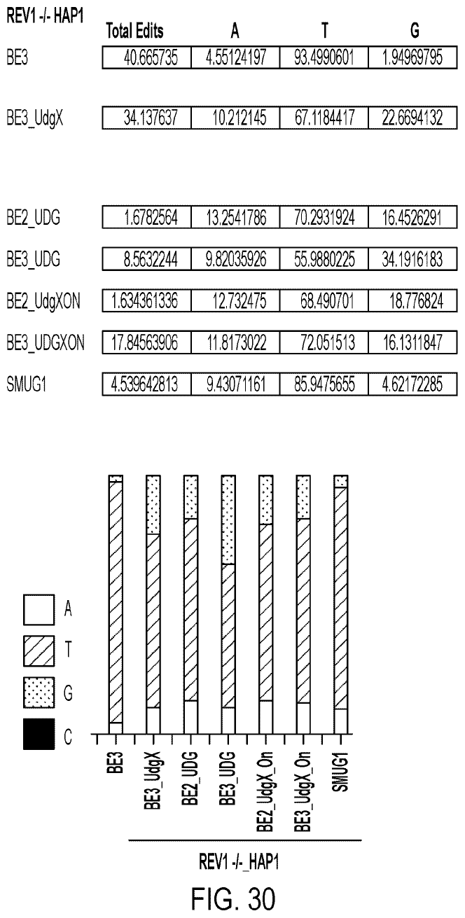

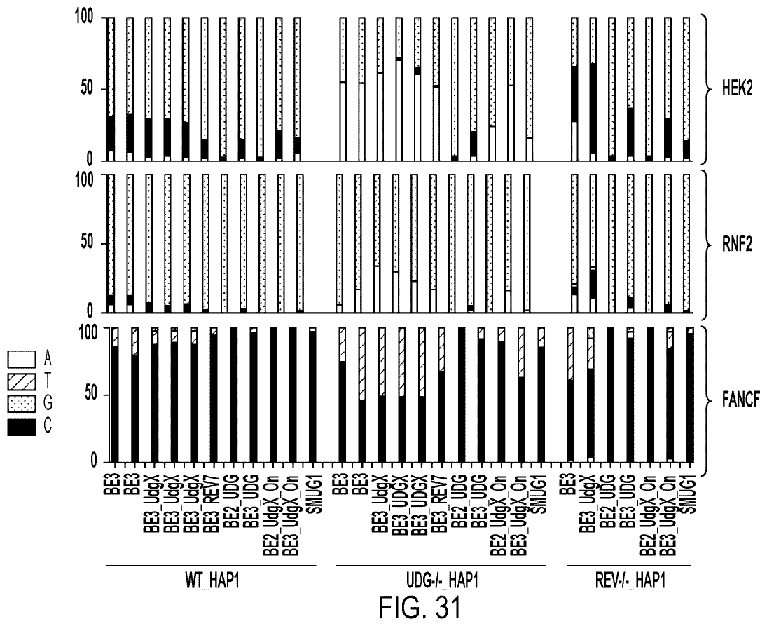

Results of C to G base editing at HEK2, RNF2, and FANCF sites in REV1^{-/-} cells using various C to G base editors (BE3; BE3_UdgX; BE2_UNG; BE3_UNG; BE2UdgX_On; BE3UdgX_On; and SMUG1) are shown in FIGS. 25 through 30.

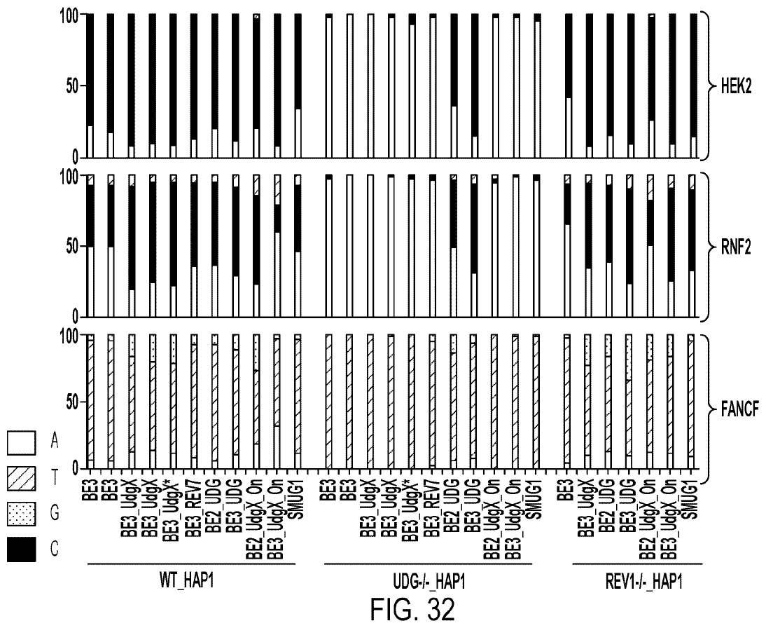

Results of C to G base editing at HEK2, RNF2, and FANCF sites in the three respective cell types (WT, UDG−/−, and REV1−/− cells) using various C to G base editors (BE3; BE3_UdgX; BE2_UNG; BE3_UNG; BE2UdgX_On; BE3UdgX_On; and SMUG1) are summarized in FIGS. 31 and 32.

### Example 2: C to G Approach 2—Increase C Incorporation Opposite an Abasic Site

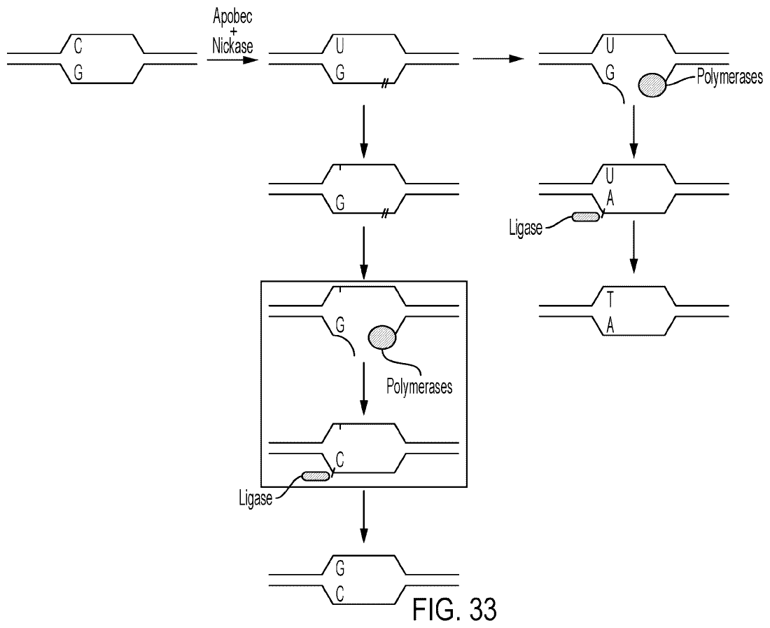

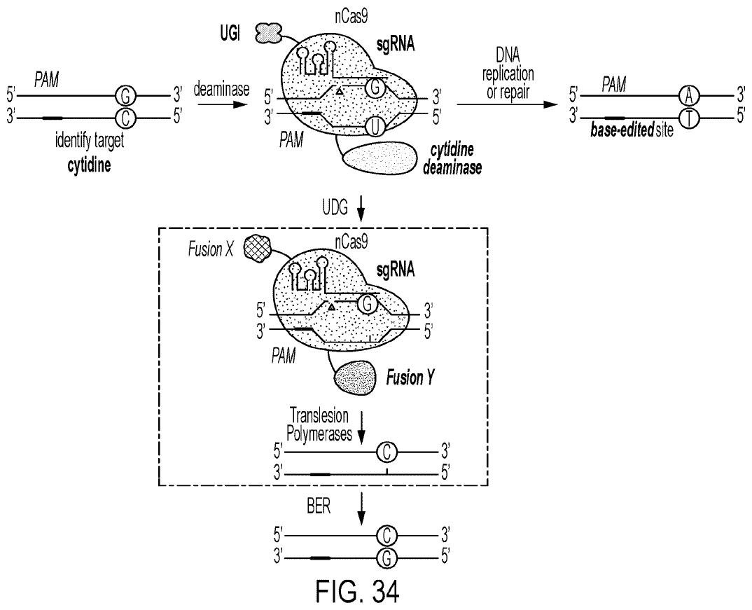

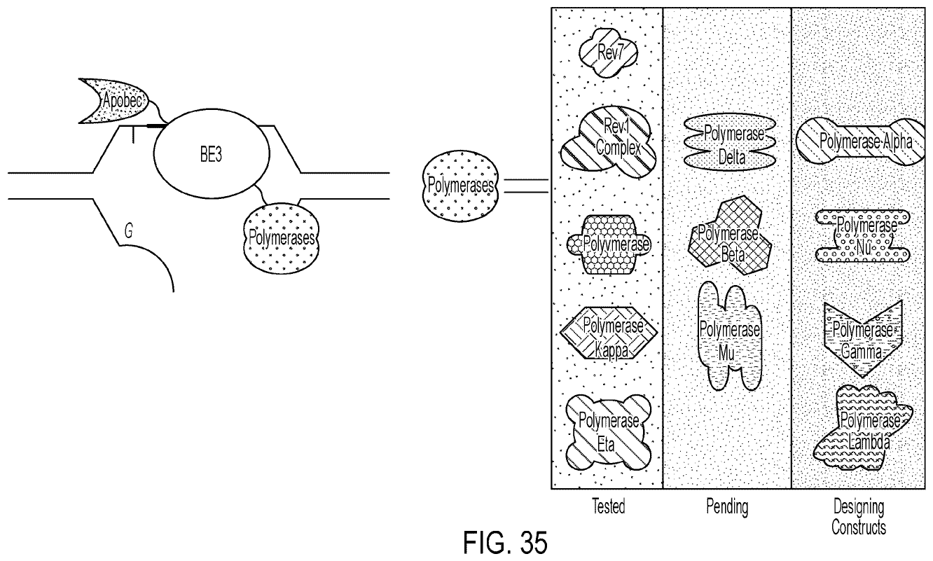

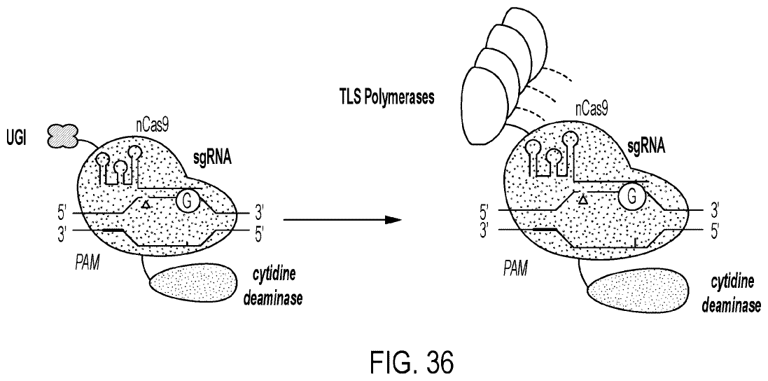

An increase in the preference for C integration opposite an abasic site should lead to an increase in total C to G base editing. A schematic for this approach and base editors used in this approach is illustrated in FIGS. 33 and 34. Various polymerases that can be used in this approach for C to G base editing are shown in FIG. 35. Briefly Abasic site generation leads to C to non-T product formation. Rev1 has dC transferase activity. Eliminating this pathway or altering how abasic lesions are repaired should lead to new base editors. Rev1−/− knockout cell lines should lack C to G editing if this pathway is solely responsible for formation of this product. The fusion of various polymerases should lead to repair of the opposite strand based on polymerase preference for repair opposite an abasic sites leading to increased C to G base editing. Exemplary base editors are illustrated in FIG. 36.

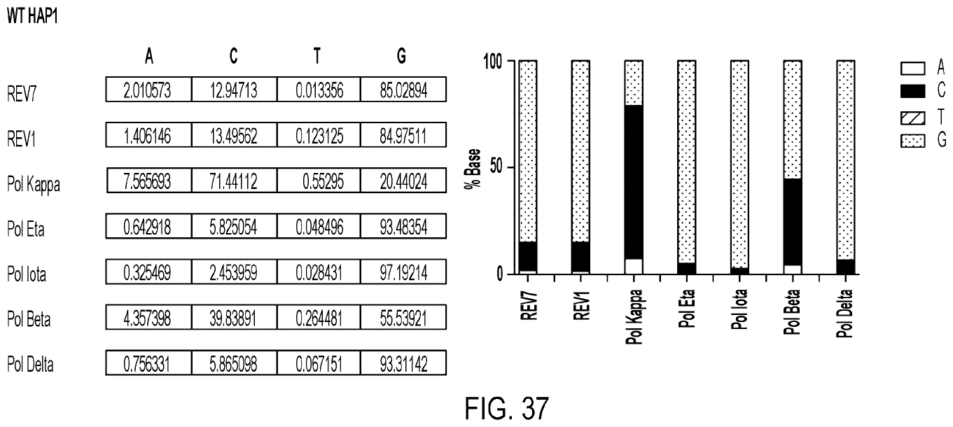

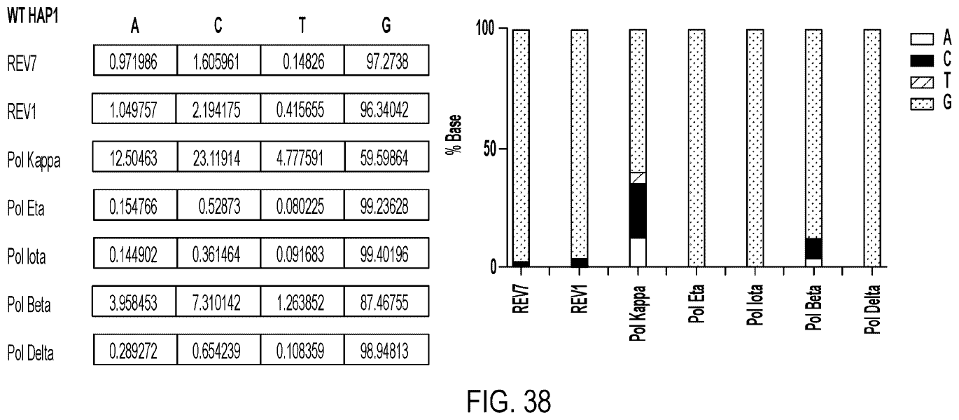

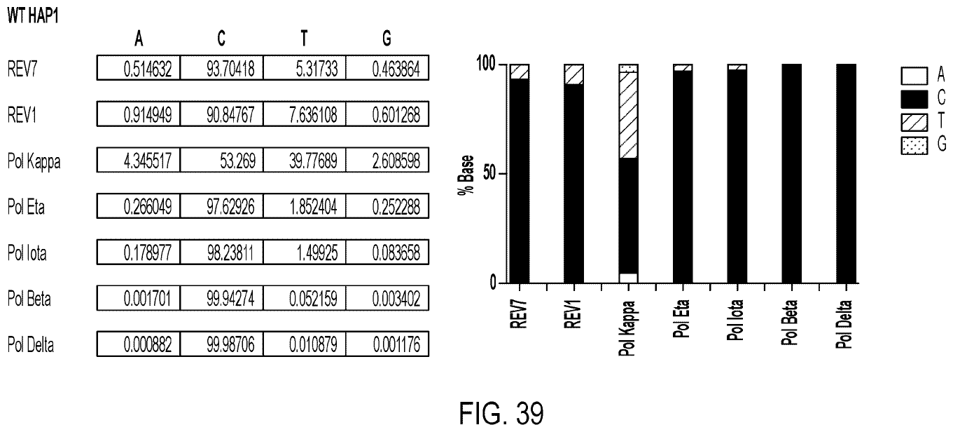

Results of C to G base editing at HEK2, RNF2, and FANCF sites in WT cells using various base editors (BE3; BE3_UdgX; BE2_UdgX_On; BE3_UdgX_On; BE2_UDG; and BE3_UDG) are shown in FIGS. 37 through 39.

Steady-state Kinetic parameters for one-base incorporation opposite an abasic site and G by human polymerases f, t, x, and REV1 are given in Table 2. See, Choi et al. J mol Bio. 2010).

TABLE 2
Steady-state Kinetic parameters for polymerases η, ι, κ, and REV1

| Poly- | | | | | k_{cat}/Kₘ | dNTP | Relative |
|---|---|---|---|---|---|---|---|
| merase | Template | dNTP | Kₘ (μM) | k_{cat} (s⁻¹) | (mM⁻¹ s⁻¹) | selectivity ratio^{a} | efficiency^{b} |
| η | AP site | A | 40 ± 6 | 0.12 ± 0.004 | 3.0 | 0.95 | 0.065 |
| | | T | 290 ± 50 | 0.92 ± 0.05 | 3.2 | 1 | 0.070 |
| | | G | 8.5 ± 1.0 | 0.005 ± 0.0001 | 0.59 | 0.19 | 0.013 |
| | | C | 210 ± 20 | 0.14 ± 0.01 | 0.67 | 0.21 | 0.015 |
| | G | C | 2.6 ± 0.1 | 0.12 ± 0.005 | 46 | | 1 |
| ι | AP site | A | 210 ± 40 | 0.54 ± 0.04 | 2.6 | 0.45 | 1.4 |
| | | T | 130 ± 20 | 0.74 ± 0.02 | 5.7 | 1 | 3.0 |
| | | G | 120 ± 10 | 0.47 ± 0.01 | 3.9 | 0.69 | 2.1 |
| | | C | 570 ± 140 | 0.77 ± 0.05 | 1.4 | 0.24 | 0.74 |
| | G | C | 300 ± 30 | 0.57 ± 8.02 | 1.9 | | 1 |
| κ | AP site | A | 1600 ± 200 | 0.077 ± 0.005 | 0.048 | 0.77 | 0.00065 |
| | | T | 2300 ± 700 | 0.017 ± 0.002 | 0.0074 | 0.12 | 0.00010 |
| | | G | 400 ± 70 | 0.0032 ± 0.0002 | 0.008 | 0.13 | 0.00011 |
| | | C | 780 ± 220 | 0.049 ± 0.005 | 0.063 | 1 | 0.00085 |
| | G | C | 3.8 ± 0.5 | 0.28 ± 0.01 | 74 | | 1 |
| REV1 | AP site | A | 140 ± 50 | 0.000025 ± 0.000002 | 0.00018 | 0.8031 | 0.00019 |
| | | T | 190 ± 30 | 0.000072 ± 0.000003 | 0.00038 | 0.0067 | 0.00040 |
| | | G | 190 ± 50 | 0.000031 ± 0.000003 | 0.00016 | 0.0029 | 0.00017 |
| | | C | 210 ± 30 | 0.012 ± 0.001 | 0.057 | 1 | 0.061 |
| | G | C | 12.8 ± 50 | 0.012 ± 0.0003 | 0.94 | | 1 |

^{a}dNTP selectivity ratio, calculated by dividing k_{cat}/Kₘ for each dNTP incorporation by the highest k_{cat}/Kₘ for dNTP incorporation opposite AP site.
bRelative efficiency, calculated by divifing k_{cat}/K_{m f}or each dNTP incorporation opposite AP site by k_{cat}/Kₘ for dCTP incorporation opposite G.

Steady-state kinetic parameters for one-base incorporation opposite an abasic site and G by human polymerases a and 6/PCNA are given in Table 3.

TABLE 3
Steady-state Kinetic parameters for polymerase α and δ/PCNA
Steady-state kinetic parameters for one-base incorporation opposite an AP site and G by human pols α and δ/PCNA

| Poly- | | | | | k_{cat}/Kₘ | dNTP | Relative |
|---|---|---|---|---|---|---|---|
| merase | Template | dNTP | Kₘ (μM) | k_{cat} (s⁻¹) | (mM⁻¹ s⁻¹) | selectivity ratio^{a} | efficiency^{b} |
| α | AP site | A | 570 ± 100 | 0.0083 ± 0.0001 | 0.015 | 1 | 0.0010 |
| | | T | 250 ± 60 | 0.00046 ± 0.00003 | 0.0018 | 0.12 | 0.00012 |
| | | G | 550 ± 120 | 0.00024 ± 0.00002 | 0.0004 | 0.027 | 0.00003 |
| | | C | 980 ± 50 | 0.00047 ± 0.000001 | 0.0005 | 0.033 | 0.00003 |
| | G | C | 0.42 ± 0.09 | 0.0064 ± 0.0003 | 15 | 1 | 1 |
| δ/PCNA | AP site | A | 25 ± 6 | 0.0067 ± 0.0004 | 0.27 | 0.36 | 0.012 |
| | | T | 62 ± 16 | 0.0060 ± 0.0004 | 0.097 | 0.34 | 0.0044 |
| | | G | 110 ± 20 | 0.010 ± 0.001 | 0.091 | 0.029 | 0.0041 |
| | | C | 880 ± 160 | 0.0069 ± 0.0006 | 0.0078 | | 0.0004 |
| | G | C | 0.27 ± 0.05 | 0.0059 ± 0.0002 | 22 | | 1 |

^{a}dNTP selectivity ratio, calculated by dividing k_{cat}/Kₘ for each dNTP incorporation by the highest k_{cat}/Kₘ for dNTP incorporation opposite AP site.
bRelative efficiency, calculated by dividing k_{cat}/Kₘ for each dNTP incorporation opposite AP site by k_{cat}/Kₘ for dCTP incorporation opposite G.

TABLE 4
Polymerases that can be used for base editing approach 2.

| | |
|---|---|
| Polymerase | Size (Amino Acids) |

Family X

| | |
|---|---|
| Beta | 335 |
| Lambda | 575 |
| Mu | 494 |

Family B

| | |
|---|---|
| Alpha | 1462 |
| Delta | 1107 |
| Epsilon | 2286 |

Family Y

| | |
|---|---|
| Eta | 713 |
| Iota | 740 |
| Kappa | 870 |
| Rev1 | 1251 |
| Zeta (Rev3/Rev7) | 3130 |

### Example 3: C to G Approach 3—Increase Both Abasic Site Formation and C Incorporation

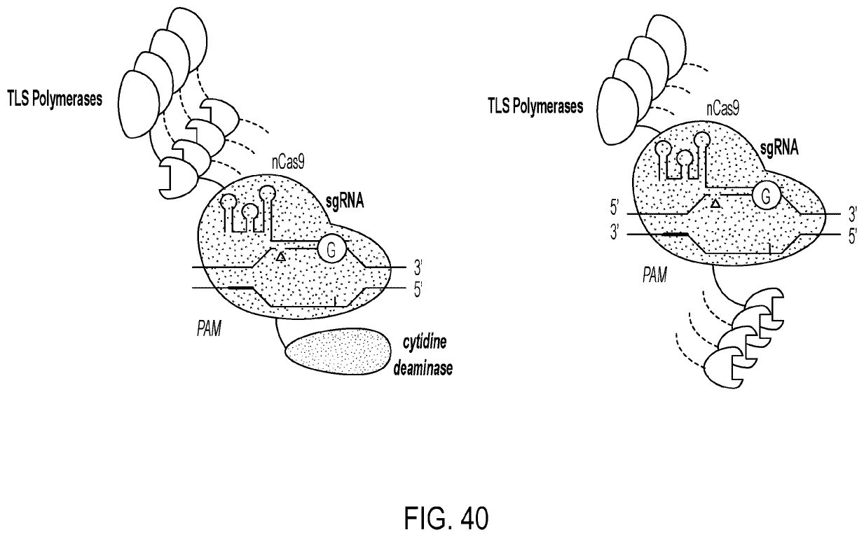

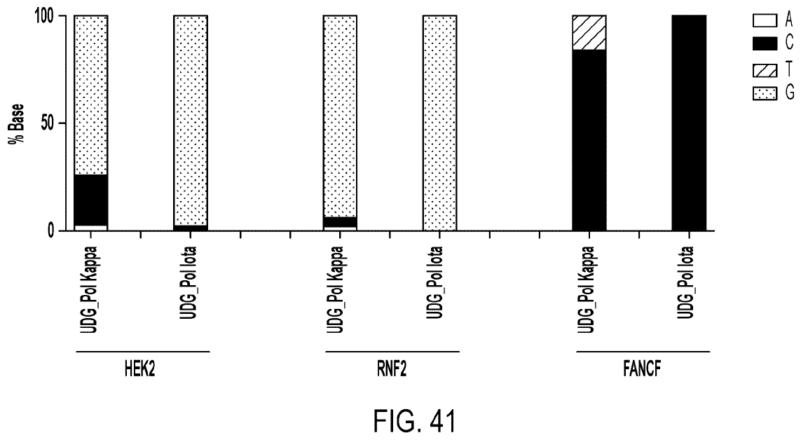

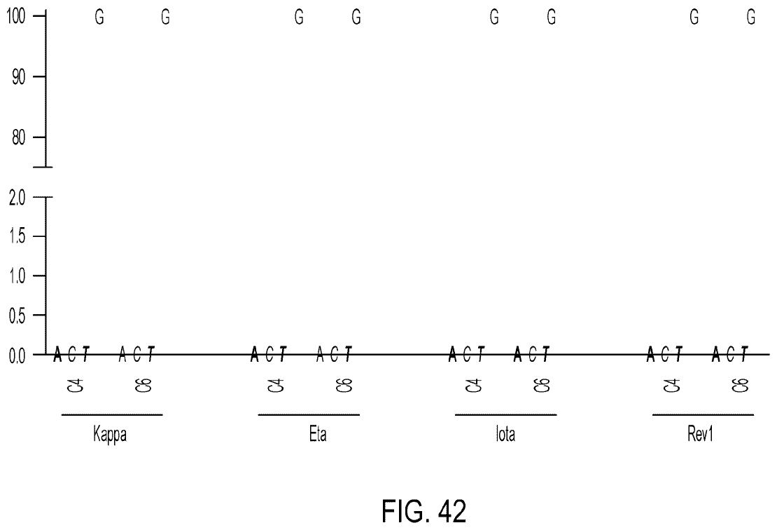

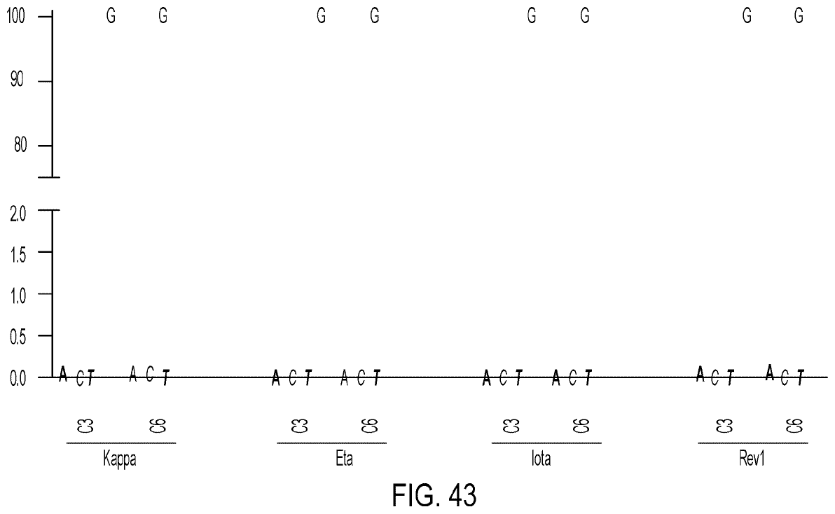

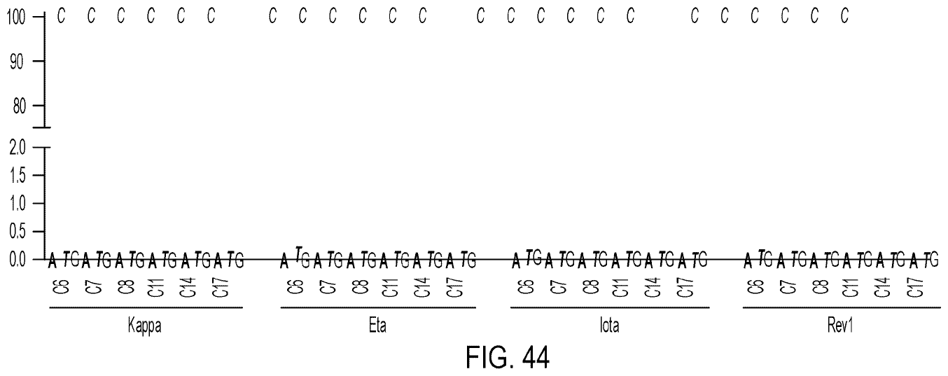

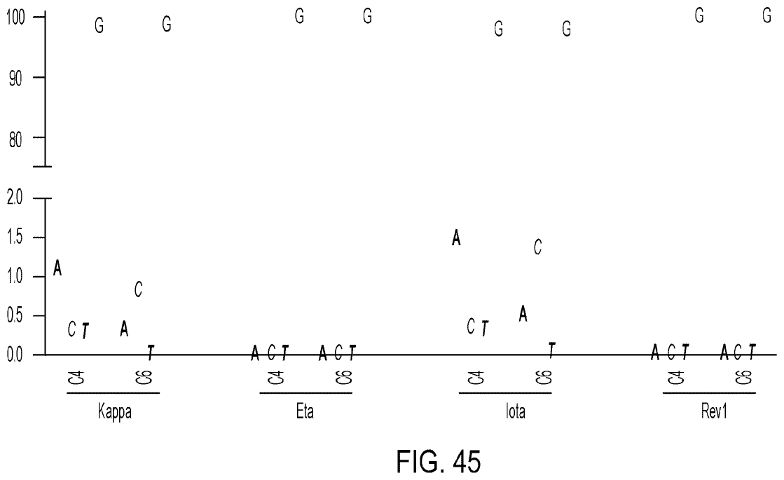

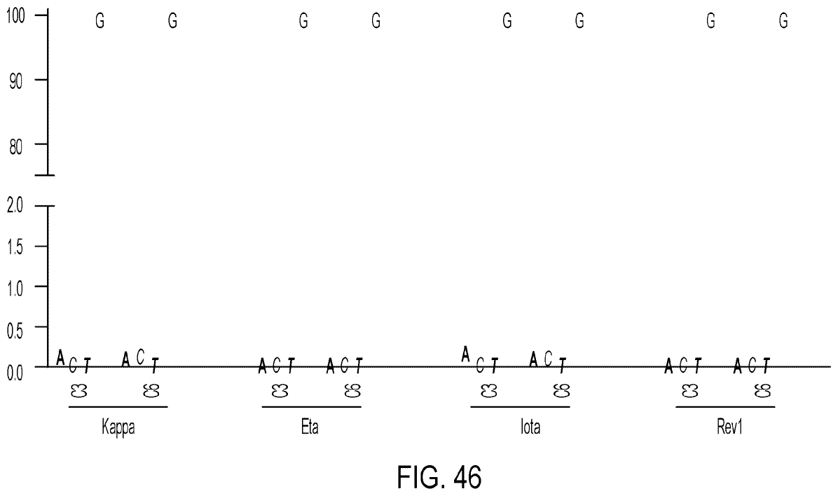

A schematic of a base editor for increasing both abasic site formation and C incorporation for increased C to G base editing is illustrated in FIG. 40. Addition of polymerase tethered constructs, particularly Pol Kappa, increases C to G base editing. Results of base editing at the HEK2, RNF2, and FANCF sites using either Pol Kappa for Pol Iota tethered constructs is shown in FIG. 41. Results of base editing using additional polymerase tethered constructs in WT cells at cytosine residues in the HEK2, RNF2, and FANCF sites are shown in FIGS. 42 through 47. UDG 147 is an enzyme that directly removes T and increases the C to G base editing (FIGS. 42 through 44), while UDG 204 is an enzyme that directly removes C and increases C to G base editing (FIGS. 45 through 47).

### Example 4: C to G Approach 4—Eliminate Alternative Repair Pathways to Increase C to G Flux

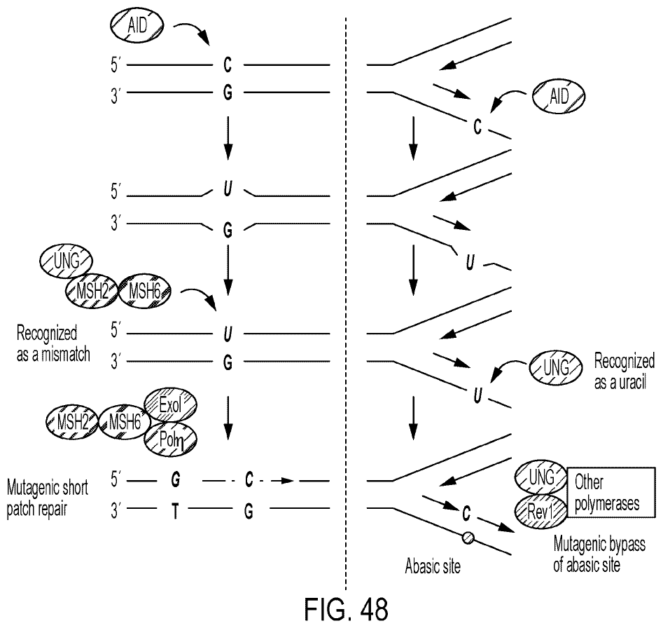

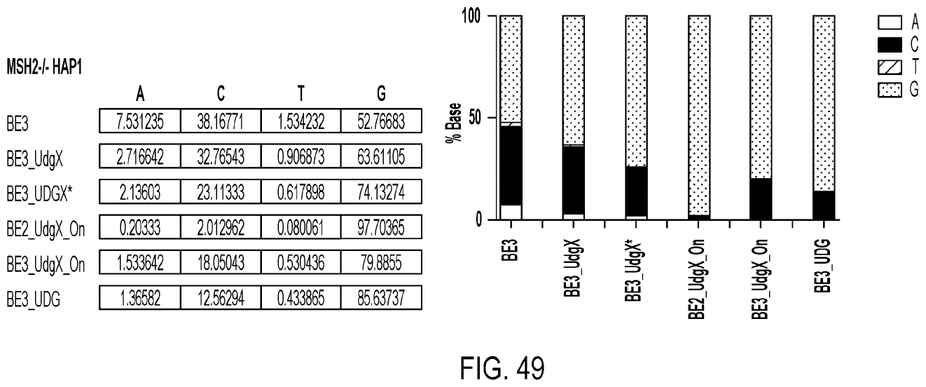

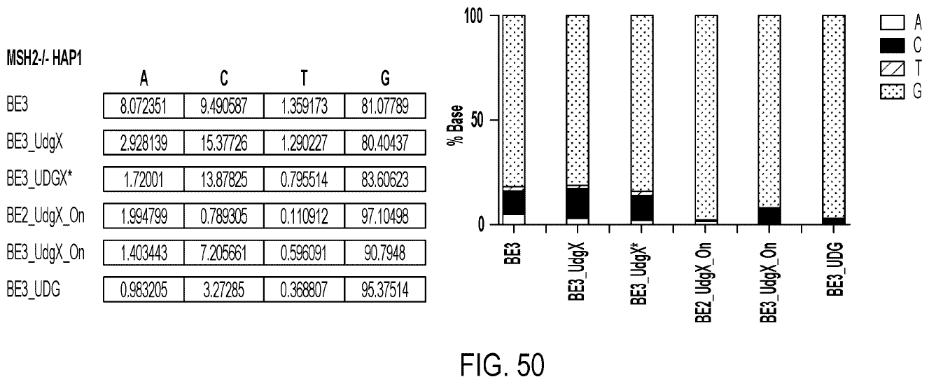

One way to improve C to G editing is to eliminate or downmodulate alternative repair pathways. AS one example, eliminating the repair pathway protein MSH2^{-/-} may lead to an increase in C to G base editing is shown in FIG. 48. The results of C to G base editing at HEK2, RNF2, and FANCF sites in MSH2^{-/-} cells using various base editors (BE3; BE3_UdgX; BE2_UdgX_On; BE3_UdgX_On; BE2_UDG; and BE3_UDG) are shown in FIGS. 49 through 51.

### Example 5: C to G Approach 5—Expression of Components in Trans

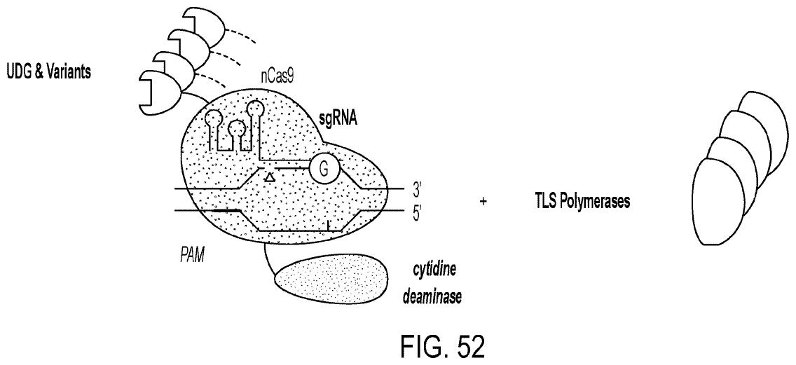

One approach for identifying base editor components that function together is to express those components together in a cell, in trans. Once base editor components (e.g., polymerases, uracil binding proteins, base excision enzymes, cytidine deaminases, and/or nucleic acid programmable DNA binding proteins) that induce C to G mutations are identified, they can be tethered to generate base editors. Expressed UDG and UdgX variants fused to APOBEC-Cas9 nickase and simultaneously overexpressed TLS polymerases in trans lead to C to G editing at the RNF2 site. A schematic illustrating the expression of components in trans is shown in FIG. 52.

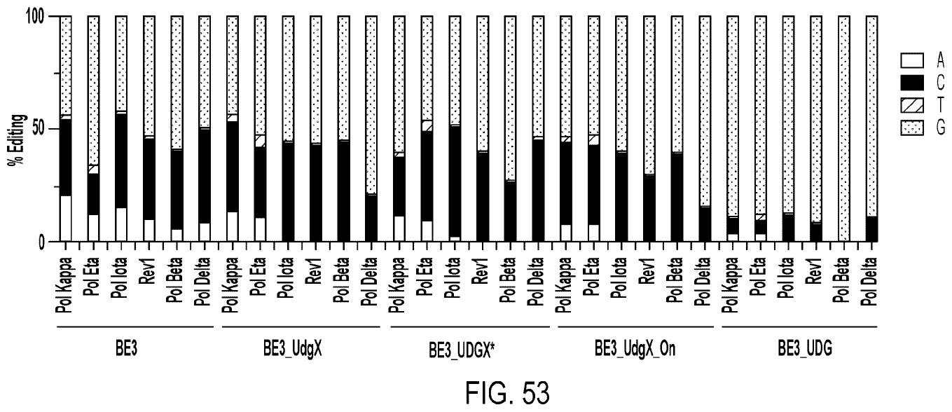

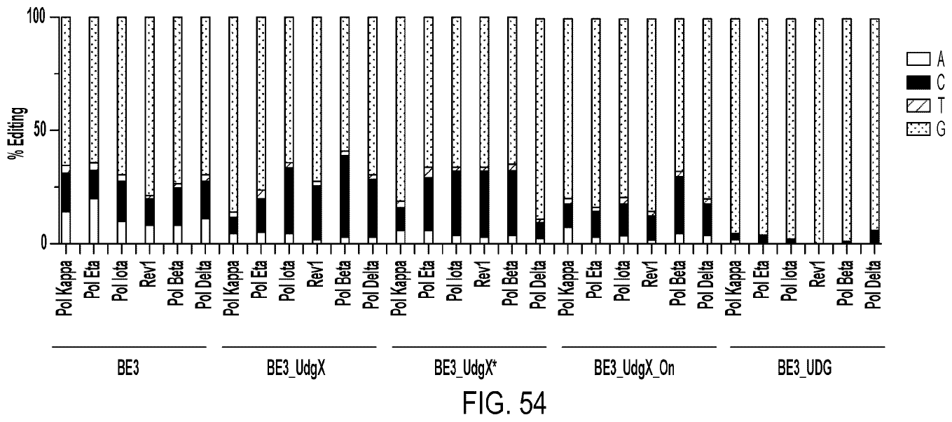

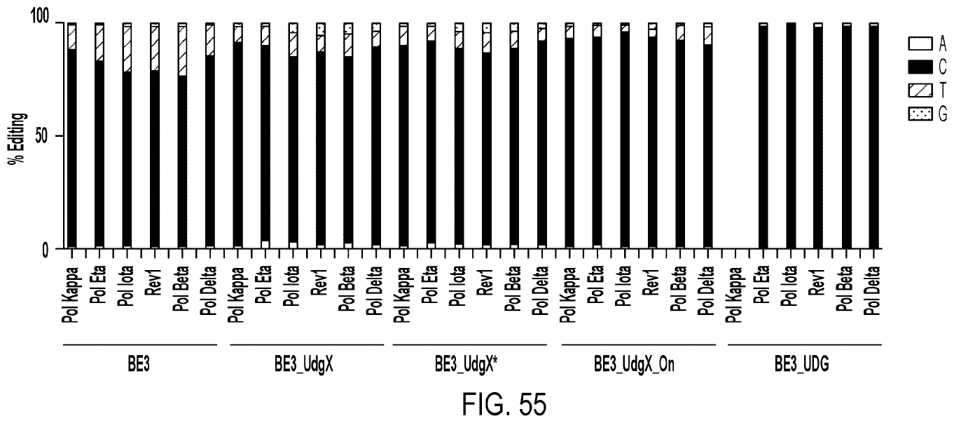

Results of base editing at HEK2, RNF2, and FANCF in HEK293 cells using five different base editors (BE3; BE3_UdgX; BE2_UdgX_On; BE3_UdgX_On; BE2_UDG; and BE3_UDG) expressed, in trans, with various polymerases (Pol Kappa, Pol Eta, Pol Iota, REV1, Pol Beta, and Pol Delta) are shown in FIGS. 53 through 55.

### REFERENCES FOR EXAMPLES 1-5

1. Chan, K., Resnick, M. A., Gordenin, D. A. The choice of nucleotide inserted opposite abasic sites formed within chromosomal DNA reveals the polymerase activities participating in translesion DNA synthesis. DNA Repair 12, 878-889 (2013).
2. Choi, J. Y., Lim, S., Kim, E. J., Jo, A., and Guengerich F. P. Translesion synthesis across abasic lesions by human B-family and Y-family DNA polymerases alpha, delta, eta, iota, kappa, and Rev 1. Journal of Molecular Biology 404, 34-44 (2010).
3. Dianov, G. L. and Hubsher U. Mammalian base excision repair: the forgotten archangel. Nucleic Acids Research, 1-8 (2013).
4. Fortini, P., Pasucci, B., Sobol, R. W., Wilson, S. H., and Dogliotti, E. Different DNA polymerases are involved in the Short- and lon-patch base excision repair in mammalian cells. Biochemistry 37, 3575-3580 (1998).
5. Jiricny, J. The multifaceted mismatch-repair system. Nature Rev. Molecular Cell Biology 7, 335-346 (2006).
6. Katafuchi A. and Nohmi T. DNA polymerases involved in the incorporation of oxidized nucelotides into DNA: their efficiency and template base preference. Mutation Research 703, 24-31 (2010).
7. Kavli, B., Slupphaug, G., Mol, C. D., Arvai, A. S., Peterson, S. B., Tainer, J. A., and Krokan, E. H. Excision of cytosine and thymine from DNA by mutants of human uracil-DNA glycosylase. EMBO 15, 3442-3447 (1996).
8. Krokan, H. E. and Bjoras, M. Base Excision Repair, Cold Spring Harbor Perspectives in Biology, 1-22 (2013).
9. Kunkel, T. A. and Erie, D. A. Eukaryotic mismatch repair in relation to RNA replication. Annual Reviews Genetics 49, 291-313 (2015).
10. Li, G. M. Mechanisms and functions of DNA mismatch repair. Cell Research 18, 85-98 (2008).
11. Lin, W., Xin, H., Wu, X., Yuan, F., and Wang, Z. The human REV1 gene codes for a DNA template-dependent dCMP transferase. Nucleic Acids Research 27, 4468-4475 (1999).
12. Mol, C. D., Arvai, A. S., Slupphaug, G., Kavil, B., Alseth, I., Krokan, H. E., and Tainer, J. A. Crystal structure and mutational analysis of human uracil-DNA glycosylase: structural basis for specificity and catalysis. Cell 80, 869-878 (1995).
13. Prasad, R., Poltoratsky, V., Hou, E. W., and Wilson, S. H. Rev1 is a base excision repair enzyme with 5′deoxyribose phosphate lyase activity. Nucleic Acid Research, 1-10 (2016).
14. Robertson, A. B., Klungland, A., Rognes, T., and Leiros, I. Base excision repair: the long and the short of it. Cell Molecular Life Sciences 66, 981-993 (2009).
15. Sale, J. E., Lehmann, A. R., and Woodgate, R. Y-Family DNA polymerases and their role in tolerance of cellular DNA damage. Nature Rev. Molecular Cell Biology 13, 141-152 (2012).
16. Sang, P. B., Srinath, T., Patil, A. G., Woo, E. J., and Varshney, U. A unique uracil-DNA binding protein of the uracil DNA glycosylase superfamily. Nucleic Acids Research, 1-12 (2015).
17. Savva, R., McAuley-Hecht, K., Brown, T., and Pearl, L. The structural basis of specific base-excision repair by uracil-DNA glycosylase. Nature 373, 487-493 (1995).
18. Slupphaug, G., Mol, C. D., Kavli, B., Arvai, A. S., Krokan, H. E., and Tainer, J. A. A nucleotide-flipping mechanism from the structure of human uracil-DNA glycosylase bound to DNA. Nature 384, 87-92 (1996).
19. Weill, J. C. and Reynaud C. A. DNA polymerases in adaptive immunity. Nature Rev. Immunology 8, 302-312 (2008).
20. Yasui, A. Alternative excision repair pathways. Cold Spring Harbor Perspectives in Biology, 1-8 (2013).

### Example 6:—Cas9 Variant Sequences

The disclosure provides Cas9 variants, for example Cas9 proteins from one or more organisms, which may comprise one or more mutations (e.g., to generate dCas9 or Cas9 nickase). In some embodiments, one or more of the amino acid residues, identified below by an asterek, of a Cas9 protein may be mutated. In some embodiments, the D10 and/or H840 residues of the amino acid sequence provided in SEQ ID NO: 6, or a corresponding mutation in any Cas9 provided herein, such as any one of the amino acid sequences provided in SEQ ID NOs: 4-26, are mutated. In some embodiments, the D10 residue of the amino acid sequence provided in SEQ ID NO: 6, or a corresponding mutation in any Cas9 provided herein, such as any of the amino acid sequences provided in SEQ ID NOs: 4-26, is mutated to any amino acid residue, except for D. In some embodiments, the D10 residue of the amino acid sequence provided in SEQ ID NO: 6, or a corresponding mutation in any Cas9, such as any one of the amino acid sequences provided in SEQ ID NOs: 4-26, is mutated to an A. In some embodiments, the H840 residue of the amino acid sequence provided in SEQ ID NO: 6, or a corresponding residue in any Cas9, such as any of the amino acid sequences provided in SEQ ID NOs: 4-26, is an H. In some embodiments, the H840 residue of the amino acid sequence provided in SEQ ID NO: 6, or a corresponding mutation in any Cas9, such as any of the amino acid sequences provided in SEQ ID NOs: 4-26, is mutated to any amino acid residue, except for H. In some embodiments, the H840 residue of the amino acid sequence provided in SEQ ID NO: 6, or a corresponding mutation in any Cas9, such as any of the amino acid sequences provided in SEQ ID NOs: 4-26, is mutated to an A. In some embodiments, the D10 residue of the amino acid sequence provided in SEQ ID NO: 6, or a corresponding residue in any Cas9, such as any of the amino acid sequences provided in SEQ ID NOs: 4-26, is a D.

Cas9 sequences from various species were aligned to determine whether corresponding homologous amino acid residues of D10 and H840 of SEQ ID NO: 6 can be identified in other Cas9 proteins, allowing the generation of Cas9 variants with corresponding mutations of the homologous amino acid residues. The alignment was carried out using the NCBI Constraint-based Multiple Alignment Tool (COBALT (accessible at st-va.ncbi.nlm.nih.gov/tools/cobalt), with the following parameters. Alignment parameters: Gap penalties −11,−1; End-Gap penalties −5,−1. CDD Parameters: Use RPS BLAST on; Blast E-value 0.003; Find Conserved columns and Recompute on. Query Clustering Parameters: Use query clusters on; Word Size 4; Max cluster distance 0.8; Alphabet Regular.

An exemplary alignment of four Cas9 sequences is provided below. The Cas9 sequences in the alignment are: Sequence 1 (S1): SEQ ID NO: 23| WP_0109222511 gi 499224711 type II CRISPR RNA-guided endonuclease Cas9 [Streptococcus pyogenes]; Sequence 2 (S2): SEQ ID NO: 24| WP_039695303 I gi 746743737| type II CRISPR RNA-guided endonuclease Cas9 [Streptococcus gallolyticus]; Sequence 3 (S3): SEQ ID NO: 25| WP_045635197 I gi 782887988| type II CRISPR RNA-guided endonuclease Cas9 [Streptococcus mitis]; Sequence 4 (S4): SEQ ID NO: 26 | 5AXW_A |gi 9244435461 Staphylococcus Aureus Cas9. The HNH domain (bold and underlined) and the RuvC domain (boxed) are identified for each of the four sequences. Amino acid residues 10 and 840 in S1 and the homologous amino acids in the aligned sequences are identified with an asterisk following the respective amino acid residue.

S1 1 --MDKK-YSIGLD*IGTNSVGWAVITDEYKVPSKKFKVLGNTDRHSIKKNLI--GALLFDSG--ETAEATRLKRTARRRYT 73
S2 1 --MTKKNYSIGLD*IGTNSVGWAVITDDYKVPAKKMKVLGNTDKKYIKKNLL--GALLFDSG--ETAEATRLKRTARRRYT 74
S3 1 --M-KKGYSIGLD*IGTNSVGFAVITDDYKVPSKKMKVLGNTDKRFIKKNLI--GALLFDEG--TTAEARRLKRTARRRYT 73
S4 1 GSHMKRNYILGLD*IGITSVGYGII--DYET-----------------RDVIDAGVRLFKEANVENNEGRRSKRGARRLKR 61
S1 74 RRKNRICYLQEIFSNEMAKVDDSFFHRLEESFLVEEDKKHERHPIFGNIVDEVAYHEKYPTIYHLRKKLVDSTDKADLRL 153
S2 75 RRKNRLRYLQEIFANEIAKVDESFFQRLDESFLTDDDKTFDSHPIFGNKAEEDAYHQKFPTIYHLRKHLADSSEKADLRL 154
S3 74 RRKNRLRYLQEIFSEEMSKVDSSFFHRLDDSFLIPEDKRESKYPIFATLTEEKEYHKQFPTIYHLRKQLADSKEKTDLRL 153
S4 62 RRRHRIQRVKKLL--------------FDYNLLTD--------------------HSELSGINPYEARVKGLSQKLSEEE 107
S1 154 IYLALAHMIKFRGHFLIEGDLNPDNSDVDKLFIQLVQTYNQLFEENPINASGVDAKAILSARLSKSRRLENLIAQLPGEK 233
S2 155 VYLALAHMIKFRGHFLIEGELNAENTDVQKIFADFVGVYNRTFDDSHLSEITVDVASILTEKISKSRRLENLIKYYPTEK 234
S3 154 IYLALAHMIKYRGHFLYEEAFDIKNNDIQKIFNEFISIYDNTFEGSSLSGQNAQVEAIFTDKISKSAKRERVLKLFPDEK 233
S4 108 FSAALLHLAKRRG----------------------VHNVNEVEEDT---------------------------------- 131
S1 234 KNGLFGNLIALSLGLTPNFKSNFDLAEDAKLQLSKDTYDDDLDNLLAQIGDQYADLFLAAKNLSDAILLSDILRVNTEIT 313
S2 235 KNTLFGNLIALALGLQPNFKTNFKLSEDAKLQFSKDTYEEDLEELLGKIGDDYADLFTSAKNLYDAILLSGILTVDDNST 314
S3 234 STGLFSEFLKLIVGNQADFKKHFDLEDKAPLQFSKDTYDEDLENLLGQIGDDFTDLFVSAKKLYDAILLSGILTVTDPST 313
S4 132 -----GNELS------------------TKEQISRN-------------------------------------------- 144
S1 314 KAPLSASMIKRYDEHHQDLTLLKALVRQQLPEKYKEIFFDQSKNGYAGYIDGGASQEEFYKFIKPILEKM--DGTEELLV 391
S2 315 KAPLSASMIKRYVEHHEDLEKLKEFIKANKSELYHDIFKDKNKNGYAGYIENGVKQDEFYKYLKNILSKIKIDGSDYFLD 394
S3 314 KAPLSASMIERYENHQNDLAALKQFIKNNLPEKYDEVFSDQSKDGYAGYIDGKTTQETFYKYIKNLLSKF--EGTDYFLD 391
S4 145 ----SKALEEKYVAELQ-------------------------------------------------LERLKKDG------ 165
S1 392 KLNREDLLRKQRTFDNGSIPHQIHLGELHAILRRQEDFYPFLKDNREKIEKILTFRIPYYVGPLARGNSRFAWMTRKSEE 471
S2 395 KIEREDFLRKQRTFDNGSIPHQIHLQEMHAILRRQGDYYPFLKEKQDRIEKILTFRIPYYVGPLVRKDSRFAWAEYRSDE 474
S3 392 KIEREDFLRKQRTFDNGSIPHQIHLQEMNAILRRQGEYYPFLKDNKEKIEKILTFRIPYYVGPLARGNRDFAWLTRNSDE 471
S4 166 --EVRGSINRFKTSD-------YVKEAKQLLKVQKAYHQLDQSFIDTYIDLLETRRTYYEGP--GEGSPFGW-------K 227
S1 472 TITPWNFEEVVDKGASAQSFIERMTNFDKNLPNEKVLPKHSLLYEYFTVYNELTKVKYVTEGMRKPAFLSGEQKKAIVDL 551
S2 475 KITPWNFDKVIDKEKSAEKFITRMTLNDLYLPEEKVLPKHSHVYETYAVYNELTKIKYVNEQGKE-SFFDSNMKQEIFDH 553
S3 472 AIRPWNFEEIVDKASSAEDFINKMTNYDLYLPEEKVLPKHSLLYETFAVYNELTKVKFIAEGLRDYQFLDSGQKKQIVNQ 551
S4 228 DIKEW---------------YEMLMGHCTYFPEELRSVKYAYNADLYNALNDLNNLVITRDENEK---LEYYEKFQIIEN 289
S1 552 LFKTNRKVTVKQLKEDYFKKIECFDSVEISGVEDR---FNASLGTYHDLLKIIKDKDFLDNEENEDILEDIVLTLTLFED 628
S2 554 VFKENRKVTKEKLLNYLNKEFPEYRIKDLIGLDKENKSFNASLGTYHDLKKIL-DKAFLDDKVNEEVIEDIIKTLTLFED 632
S3 552 LFKENRKVTEKDIIHYLHN-VDGYDGIELKGIEKQ---FNASLSTYHDLLKIIKDKEFMDDAKNEAILENIVHTLTIFED 627
S4 290 VFKQKKKPTLKQIAKEILVNEEDIKGYRVTSTGKPEF---TNLKVYHDIKDITARKEII---ENAELLDQIAKILTIYQS 363
S1 629 REMIEERLKTYAHLFDDKVMKQLKR-RRYTGWGRLSRKLINGIRDKQSGKTILDFLKSDGFANRNFMQLIHDDSLTFKED 707
S2 633 KDMIHERLQKYSDIFTANQLKKLER-RHYTGWGRLSYKLINGIRNKENNKTILDYLIDDGSANRNFMQLINDDTLPFKQI 711
S3 628 REMIKQRLAQYDSLFDEKVIKALTR-RHYTGWGKLSAKLINGICDKQTGNTILDYLIDDGKINRNFMQLINDDGLSFKEI 706
S4 364 SEDIQEELTNLNSELTQEEIEQISNLKGYTGTHNLSLKAINLILDE------LWHTNDNQIAIFNRLKLVP--------- 428

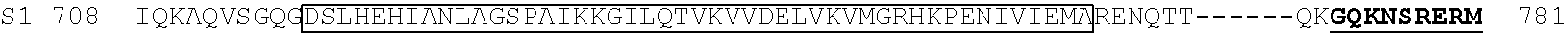

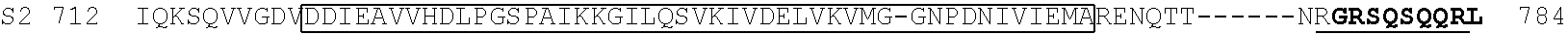

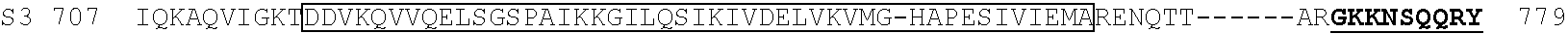

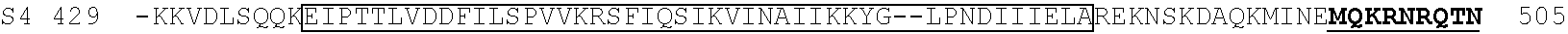

S1 782 KRIEEGIKELGSQIL-------KEHPVENTQLQNEKLYLYYLQNGRDMYVDQELDINRLSD----YDVDH*IVPQSFLKDD 850
S2 785 KKLQNSLKELGSNILNEEKPSYIEDKVENSHLQNDQLFLYYIQNGKDMYTGDELDIDHLSD----YDVDH*IVPQSFLKDD 860
S3 780 KRIEDSLKILASGL---DSNILKENPTDNNQLQNDRLFLYYLQNGKDMYTGEALDINQLSS----YDIDH*IIPQAFIKDD 861
S4 506 ERIEEIIRTTGK---------------ENAKYLIEKIKLHDMQEGKCLYSLEAIPLEDLLNNPFNYEVDH*IIPRSVSFDN 570

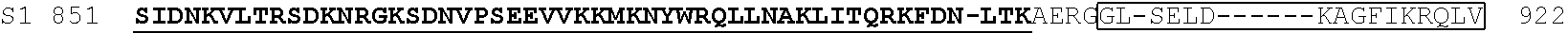

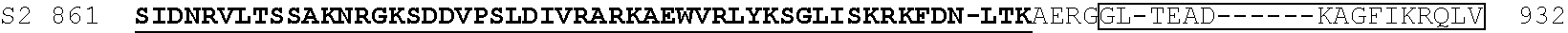

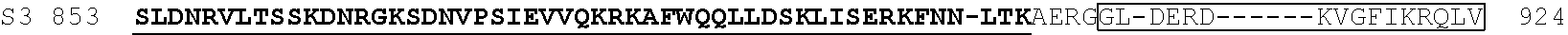

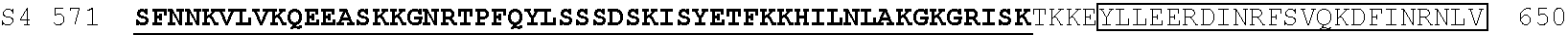

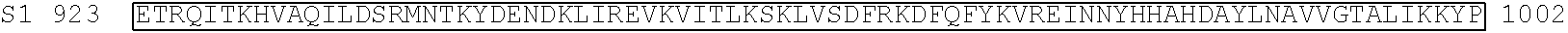

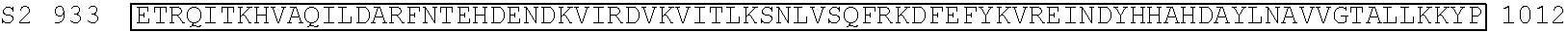

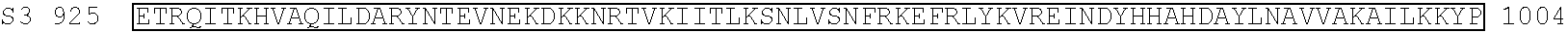

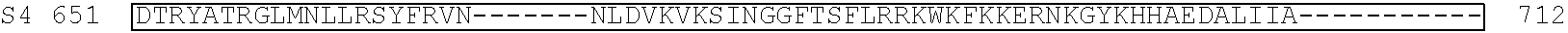

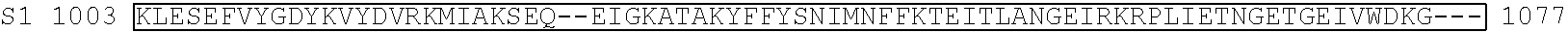

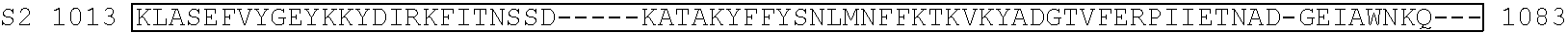

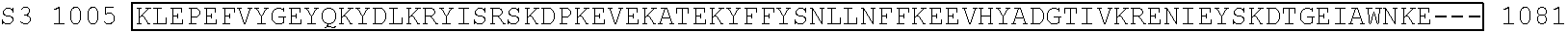

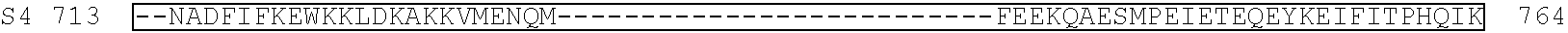

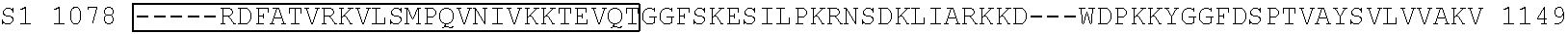

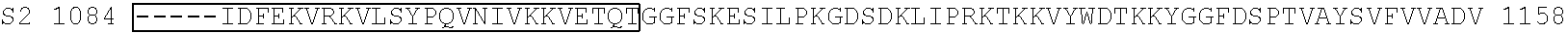

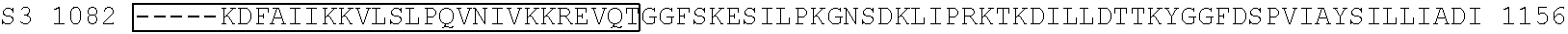

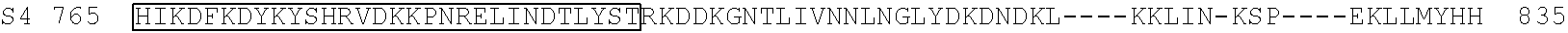

S1 1150 EKGKSKKLKSVKELLGITIMERSSFEKNPI-DFLEAKG-----YKEVKKDLIIKLPKYSLFELENGRKRMLASAGELQKG 1223
S2 1159 EKGKAKKLKTVKELVGISIMERSFFEENPV-EFLENKG-----YHNIREDKLIKLPKYSLFEFEGGRRRLLASASELQKG 1232
S3 1157 EKGKAKKLKTVKTLVGITIMEKAAFEENPI-TFLENKG-----YHNVRKENILCLPKYSLFELENGRRRLLASAKELQKG 1230
S4 836 DPQTYQKLK--------LIMEQYGDEKNPLYKYYEETGNYLTKYSKKDNGPVIKKIKYYGNKLNAHLDITDDYPNSRNKV 907
S1 1224 NELALPSKYVNFLYLASHYEKLKGSPEDNEQKQLFVEQHKHYLDEIIEQISEFSKRVILADANLDKVLSAYNKH------ 1297
S2 1233 NEMVLPGYLVELLYHAHRADNF-----NSTEYLNYVSEHKKEFEKVLSCVEDFANLYVDVEKNLSKIRAVADSM------ 1301
S3 1231 NEIVLPVYLTTLLYHSKNVHKL-----DEPGHLEYIQKHRNEFKDLLNLVSEFSQKYVLADANLEKIKSLYADN------ 1299
S4 908 VKLSLKPYRFD-VYLDNGVYKFV-----TVKNLDVIK--KENYYEVNSKAYEEAKKLKKISNQAEFIASFYNNDLIKING 979
S1 1298 RDKPIREQAENIIHLFTLTNLGAPAAFKYFDTTIDRKRYTSTKEVLDATLIHQSIT--------GLYETRI----DLSQL 1365
S2 1302 DNFSIEEISNSFINLLTLTALGAPADFNFLGEKIPRKRYTSTKECLNATLIHQSIT--------GLYETRI----DLSKL 1369
S3 1300 EQADIEILANSFINLLTFTALGAPAAFKFFGKDIDRKRYTTVSEILNATLIHQSIT--------GLYETWI----DLSKL 1367
S4 980 ELYRVIGVNNDLLNRIEVNMIDITYR-EYLENMNDKRPPRIIKTIASKT---QSIKKYSTDILGNLYEVKSKKHPQIIKK 1055
S1 1366 GGD 1368 (SEQ ID NO: 23)
S2 1370 GEE 1372 (SEQ ID NO: 24)
S3 1379 GED 1370 (SEQ ID NO: 25)
S4 1056 G-- 1056 (SEQ ID NO: 26)

The alignment demonstrates that amino acid sequences and amino acid residues that are homologous to a reference Cas9 amino acid sequence or amino acid residue can be identified across Cas9 sequence variants, including, but not limited to, Cas9 sequences from different species, by identifying the amino acid sequence or residue that aligns with the reference sequence or the reference residue using alignment programs and algorithms known in the art. This disclosure provides Cas9 variants in which one or more of the amino acid residues identified by an asterisk in SEQ ID NOs: 23-26 (e.g., S1, S2, S3, and S4, respectively) are mutated as described herein. The residues D10 and H840 in Cas9 of SEQ ID NO: 6 that correspond to the residues identified in SEQ ID NOs: 23-26 by an asterisk are referred to herein as “homologous” or “corresponding” residues. Such homologous residues can be identified by sequence alignment, e.g., as described above, and by identifying the sequence or residue that aligns with the reference sequence or residue. Similarly, mutations in Cas9 sequences that correspond to mutations identified in SEQ ID NO: 6 herein, e.g., mutations of residues 10, and 840 in SEQ ID NO: 6, are referred to herein as “homologous” or “corresponding” mutations. For example, the mutations corresponding to the D10A mutation in SEQ ID NO: 6 or S1 (SEQ ID NO: 23) for the four aligned sequences above are D11A for S2, D10A for S3, and D13A for S4; the corresponding mutations for H840A in SEQ ID NO: 6 or S1 (SEQ ID NO: 23) are H850A for S2, H842A for S3, and H560A for S4.

Further, several Cas9 sequences from different species have been aligned using the same algorithm and alignment parameters outlined above. Several Cas9 sequences (SEQ ID NOs: 11-260) from different species were aligned using the same algorithm and alignment parameters outlined above, as is shown in e.g., International Patent Publication No. WO 2017/070632, published Apr. 27, 2017, entitled “Nucleobase editors and uses thereof”; which is incorporated by reference herein. Amino acid residues homologous to residues of other Cas9 proteins may be identified using this method, which may be used to incorporate corresponding mutations into other Cas9 proteins. Amino acid residues homologous to residues 10, and 840 of SEQ ID NO: 6 were identified in the same manner as outlined above. The alignments are provided herein and are incorporated by reference. The HNH domain (bold and underlined) and the RuvC domain (boxed) are identified for each of the four sequences (SEQ ID NOs: 23-26). Single residues corresponding to amino acid residues 10, and 840 in SEQ ID NO: 6 are boxed in SEQ ID NO: 23 in the alignments, allowing for the identification of the corresponding amino acid residues in the aligned sequences.

### Example 7: Development of a Set of C•G-to-G•C Transversion Base Editors from CRISPRi Screens, Target-Library Analysis, and Machine Learning

Single-nucleotide variants (SNVs) represent approximately half of currently known human pathogenic gene variants¹. Base editors, fusions of programmable DNA-binding proteins with base-modifying enzymes, enable conversion of individual target nucleotides in the genome²⁻¹⁰. The two major classes of base editors are cytosine base editors (CBEs), which convert C•G to T•A, and adenine base editors (ABEs), which convert A•T to G•C^{2,3,8}. CBEs and ABEs can install transition mutations with high efficiency and product purity (the fraction of all edited alleles that contain only the desired edit), but in general, cannot efficiently install transversion mutations including C•G to G•C^{2,5,11,12}.

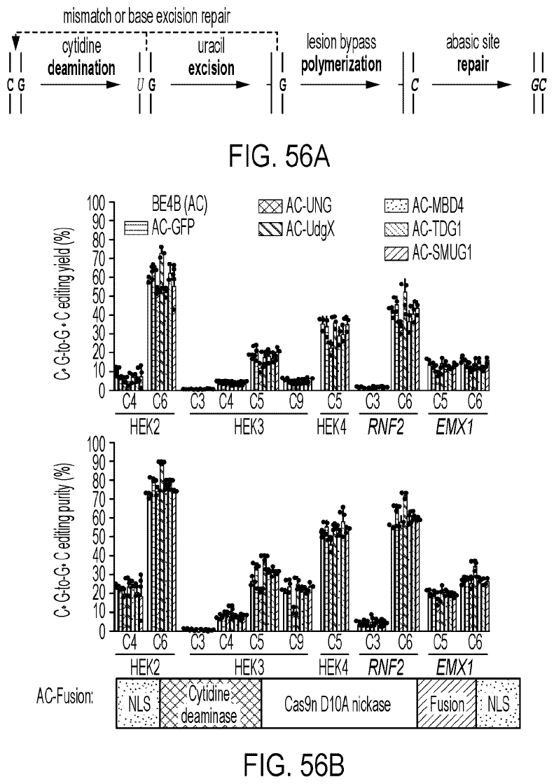

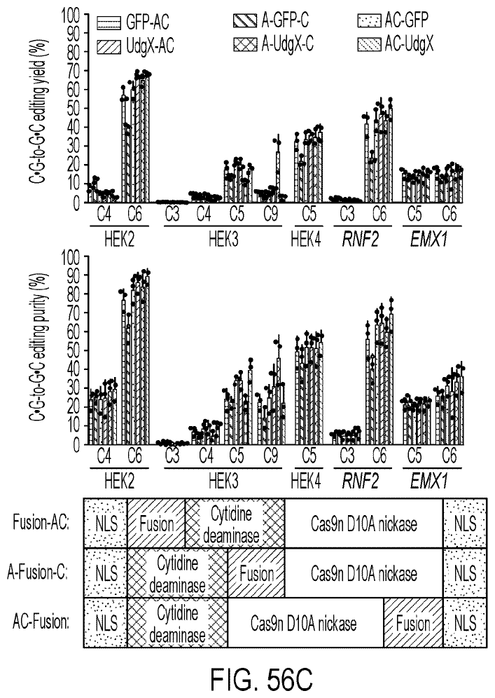

It was previously demonstrated that CBE editing byproducts, including C•G-to-G•C or C•G-to-A•T transversion outcomes, are inhibited by knockout of cellular uracil DNA N-glycosylase (UNG) or by fusion of uracil glycosylase inhibitor (UGI)^{2,7,8,11,12}, suggesting that transversion byproducts result from an abasic intermediate that is generated by UNG-catalyzed excision of deaminated target cytosines (FIG. 56A) (see International Publication No. WO 2018/165629). Consistent with this model, first-generation C•G-to-G•C base editors (CGBEs) were CBE derivatives that lack UGI domains¹¹. These CGBEs, including editors with fusions to UNG and other DNA-repair proteins¹³⁻¹⁶, can provide efficient C•G-to-G•C editing but only at a minority of tested target sites with few criteria to identify sites amenable to CGBE editing¹³⁻¹⁵.

Previously, libraries containing thousands of genomically integrated target sites and corresponding guide RNAs in mammalian cells were used to comprehensively characterize CBE and ABE base editing profiles. These data were used to train machine learning models (collectively named “BE-Hive”) that learned the sequence determinants driving CBE and ABE base editing outcomes^{12,17}. The BE-HIVE AI model provided in PCT/US2021/016924, filed Feb. 5, 2021, which is incorporated herein by reference, offered an opportunity to test how the predictions of the model hold up empirically. The BE-DICT deep learning algorithm provided in Marquart, K. F. et al. bioRxiv (2020), which is also incorporated herein by reference, offered a similar opportunity. It was envisioned that broad characterization of the sequence determinants of CGBE editing outcomes could enable accurate prediction of editing efficiencies and product purities, and thus facilitate the broader use of CGBEs.

A focused CRISPR interference (CRISPRi) screen was performed to identify DNA repair genes that impact cytosine base editing efficiency and purity. Guided by these data, various fusions proteins were constructed containing deaminases and Cas proteins fused to DNA repair components to engineer novel CGBEs with promising C•G-to-G•C editing activities. Ten such CGBEs were characterized with diverse editing profiles using a “comprehensive context library” of 10,638 genomically integrated, highly variable target sites in mouse embryonic stem cells (mESCs)¹². The resulting data was used to train machine learning models that successfully predict CGBE editing efficiency, purity, and bystander editing patterns with high accuracy (CGBE-Hive), enabling reliable identification of CGBE variants and target sites that together support high-purity C•G-to-G•C editing. Moreover, it was shown that editing activity is predicted with substantially higher accuracy by deep learning models compared to simpler models, indicating that CGBE-Hive has learned complex sequence features that play important roles in determining C-to-G editing activity. Notably, 247 cytosines predicted by CGBE-Hive to be edited by a CGBE with >80% C•G-to-G•C editing purity were indeed edited in mammalian cell experiments with an average of 83% purity.

The panel of CGBEs presented herein offer diverse editing profiles that collectively expand the sequence landscape amenable to high-quality C•G-to-G•C editing by up to 4.1-fold over the number predicted to be amenable to editing by any single CGBE. Finally, it was demonstrated that CGBE-mediated correction of 546 disease-associated single-nucleotide variants (SNVs) with >90% precision among the resulting edited amino acid sequences. These findings advance understanding of transversion base editing outcomes and provide new CGBEs that improve the scope and utility of base editing.

### Results

### Exploring the Activity of DNA Glycosylases in C•G-to-G•C Transversion Outcomes

It was previously suggested that excision of uracil from genomic DNA to generate an abasic lesion followed by error-prone polymerase activity on the strand opposite the abasic site results in C•G-to-G•C and C•G-to-A•T transversion outcomes (FIG. 56A)^{2,11,16} Motivated by this model, C•G-to-G•C base editors that enhanced uracil excision at CBE-edited nucleotides were developed. CBE architecture lacking UGI (BE4B) (BPNLS-APOBEC1-Cas9 D10A-BPNLS; abbreviated AC), was used as a starting point, similar to other reported CGBEs¹³⁻¹⁵.

A variety of known uracil excising and binding enzymes were fused to the C-terminus of the BE4B (AC) scaffold and assessed the frequency of C•G-to-G•C edits across five genomic loci in HEK293T cells (FIG. 56B). Several glycosylases (i.e., SMUG1, MBD4, and TDG2) did not alter editing outcomes, and fusion to UNG led to a reduction of C•G-to-G•C editing yield and purity at three out of five targeted sites, consistent with a recent report¹³. Nevertheless, it was found that fusion of a UNG orthologue from M. smegmatis (UdgX) moderately improved C•G-to-G•C product purity by 1.2-fold on average¹⁸⁻²⁰, with the largest improvement at the RNF2 locus (56±0.8% with BE4B to 72±2.1% with AC-UdgX; p=0.0002, Student's two-sided t-test) and significant changes observed at HEK site 2 C6, HEK site 3 C5, and EMX1 C6 (p<0.01, Student's two-sided t-test). However, only modest changes were observed to editing yield (1.1-fold relative to BE4B at the most efficiently edited C across the five tested genomic loci). These observations suggested that fusion partners may enhance C•G-to-G•C transversion base editing outcomes.

Next, the impact of orientation of the glycosylase fusion on editing outcomes was studied. BE4B (AC) fusion variants were constructed with either UdgX (abbreviated X) or GFP in three orientations: at either the N- or C-terminus (e.g., XAC or ACX) or between the deaminase and Cas9 (e.g., AXC). It was observed that C•G-to-G•C editing was similar or slightly improved for UdgX fusions compared to N- and C-terminal GFP fusions (FIG. 56C). However, the editing efficiency and purity of AXC was modestly higher than that of the best GFP fusion at a majority of sites (four out of five sites for efficiency; three out of five sites for purity). The AXC architecture was advanced since it offered similar or better performance than the XAC and ACX variants at these test loci.

### CRISPRi Screen for Determinants of Base Editing Outcomes

Next, the impact of other DNA repair or translesion synthesis factors on C•G-to-G•C editing outcomes of AXC was investigated. It was previously demonstrated that the purity of canonical C•G-to-T•A edits by CBEs improved dramatically in cells lacking nuclear uracil DNA N-glycosylase (UNG) or when one or more uracil glycosylase inhibitor proteins (UGI) were appended CBEs^{2,11,12,16}, suggesting that excision of uracil from genomic DNA to form an abasic site was an important early step in achieving transversion base editing outcomes. As such, the molecular mechanisms that transform abasic sites into transversion edits in mammalian cells were studied further.

UdgX fusion proteins were tested to determine whether they require cellular UNG to install C•G-to-G•C edits. C•G-to-G•C editing with AXC was minimal in UNG2-HAP1 cells compared to UNG+ cells, confirming that C•G-to-G•C transversion outcomes indeed are promoted by cellular UNG-mediated formation of an abasic site intermediate, even when using the AXC construct (FIG. 2A).

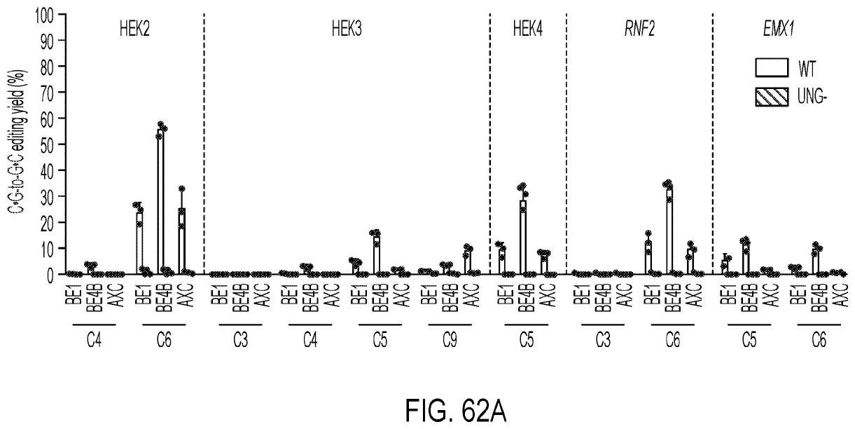

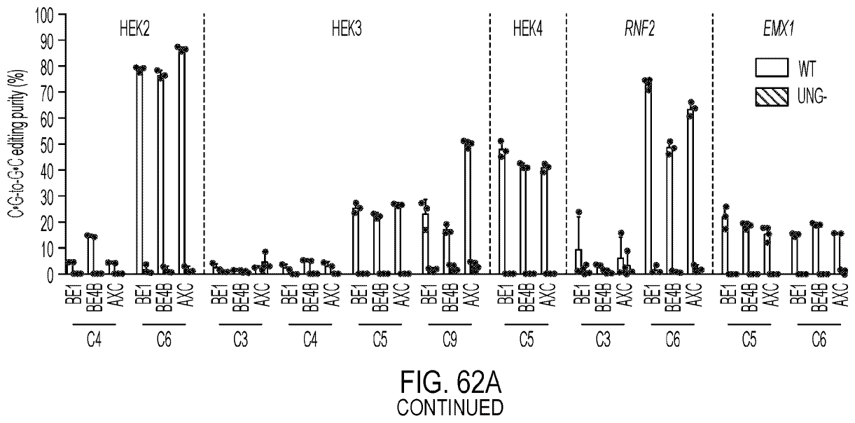

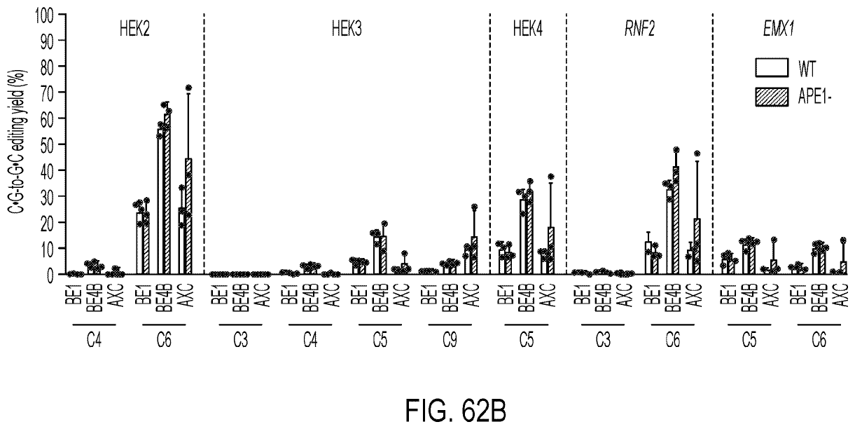

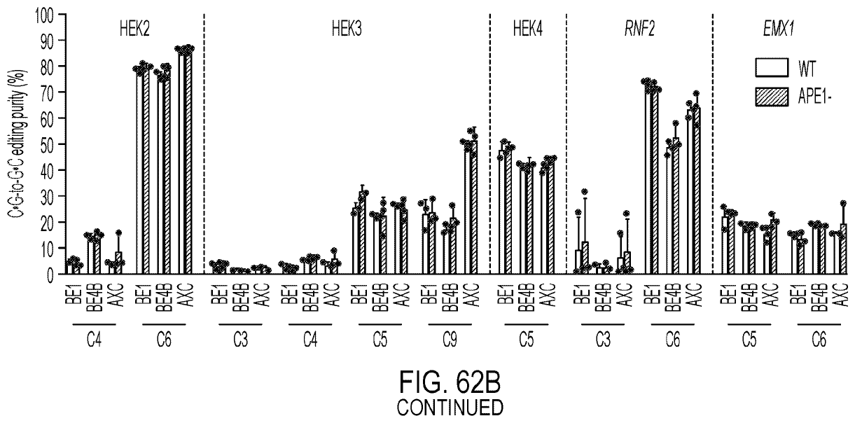

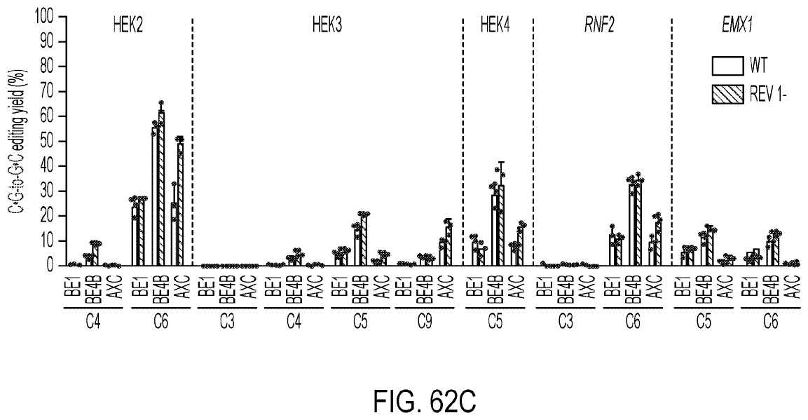

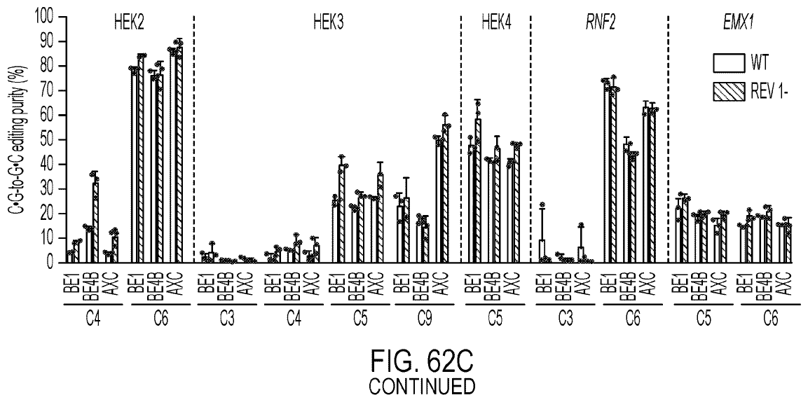

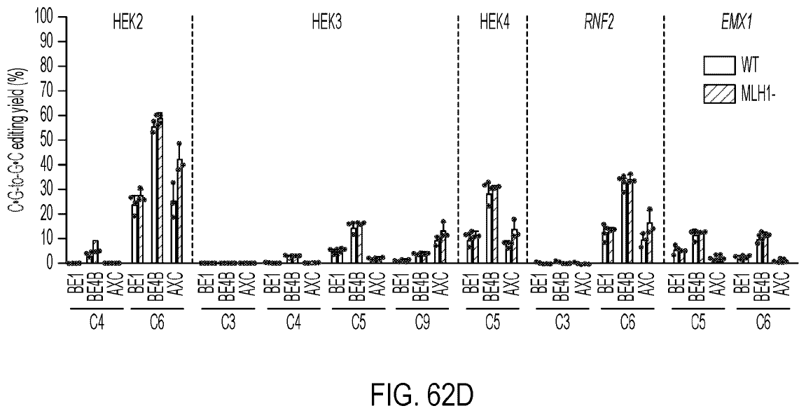

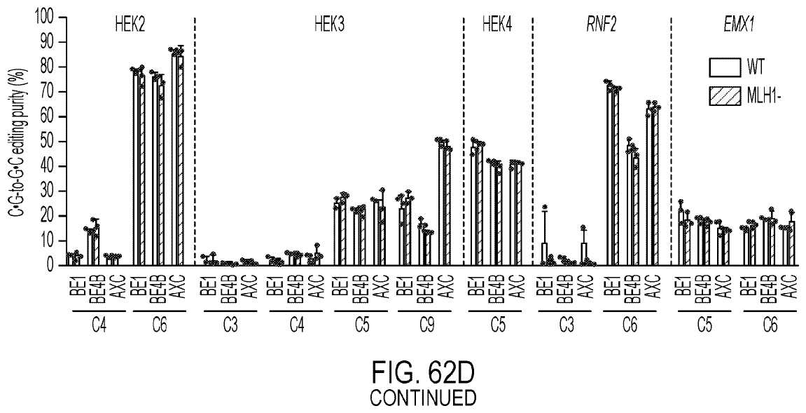

AP endonuclease-1 (APE1 or APEX1) initiates short patch base excision repair (sp-BER) following abasic site formation by nicking the abasic site-containing strand. Polymerases such as PolB then resynthesize the damaged strand using the intact stand as a template^{38,39}. Loss of APE1 was tested to determine whether it could bias the repair of CBE-induced abasic sites towards C•G-to-G•C outcomes by measuring cytosine base editing outcomes with non-nicking BE1 (BPNLS-APOBEC1-dead Cas9-BPNLS), nicking BE4B (BPNLS-APOBEC1-Cas9 D10A-BPNLS), and the AXC construct in APE1-deficient HAP1 cells. No meaningful differences in editing by BE1 in APE1-deficient HAP1 cells were observed compared to APE1+HAP1 cells. C•G-to-G•C editing yields with either BE4B or AXC were modestly increased in APE1-cells compared to APE1+ cells and C•G-to-G•C editing purity was not significantly different (FIG. 62B). These data suggest that APE1 does not play a major non-redundant role in resolving CBE edits towards transversion outcomes.

Next, the contributions of mismatch repair proteins on C•G-to-G•C editing outcomes were evaluated⁴⁰. Using the same panel of BE1, BE4B, and AXC editors, only modest changes in C•G-to-G•C editing yield and no significant changes in editing purity in MLH1-HAP1 cells compared with MLH1+ controls were observed (FIG. 62C).

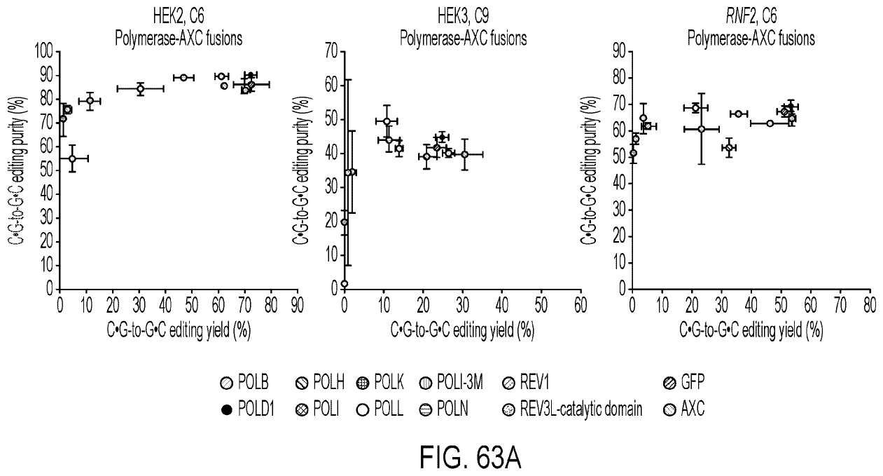

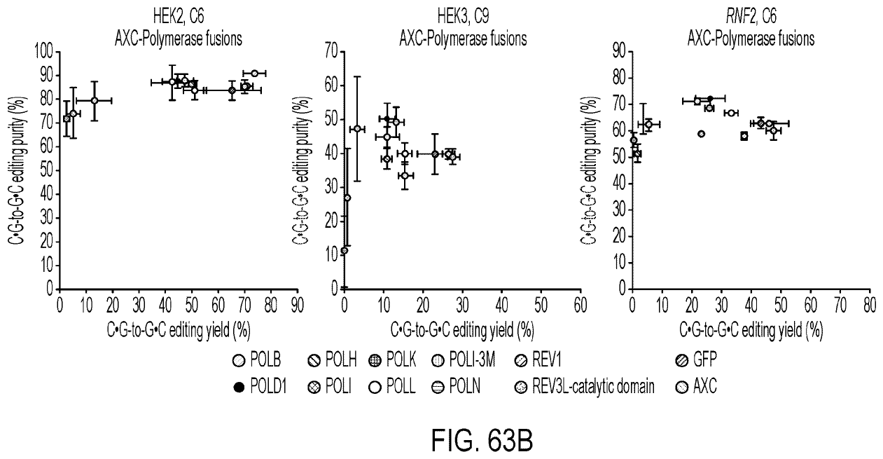

Surprisingly, loss of REV1—a cellular polymerase known for its deoxycytidyl transferase activity^{41,42}—modestly increased, rather than decreased, C•G-to-G•C editing outcomes. These data suggest that alternative polymerases could install C opposite abasic lesions that result from cytosine base editing. (FIG. 62D). To explore the possibility that other polymerases may play key roles in installing either the C opposite the abasic site or the G that replaces the original C, a panel of ten N- and C-terminal fusions of DNA polymerase catalytic domains to the AXC construct were constructed and assessed editing outcomes at three genomic loci in HEK293T cells. No consistently improved editing outcomes were observed with any polymerase-fused AXC variant^{39,43} (FIGS. 63A-63D).

No significant changes in editing purity of AXC was observed in individual UNG, APE1/APEX1, MLH1, REV1 knockout cell lines, and direct AXC fusions to mammalian polymerase domains did not consistently improve editing outcomes (FIGS. 62A-62D and FIGS. 63A-63B). Thus, a much broader search for modulators of cytosine transversion editing was performed by performing two high-throughput genetic screens.

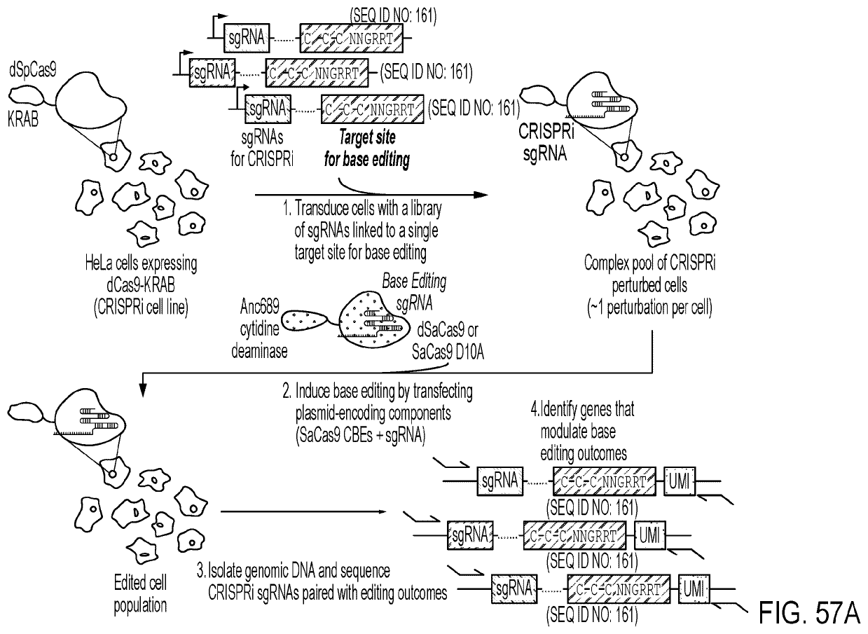

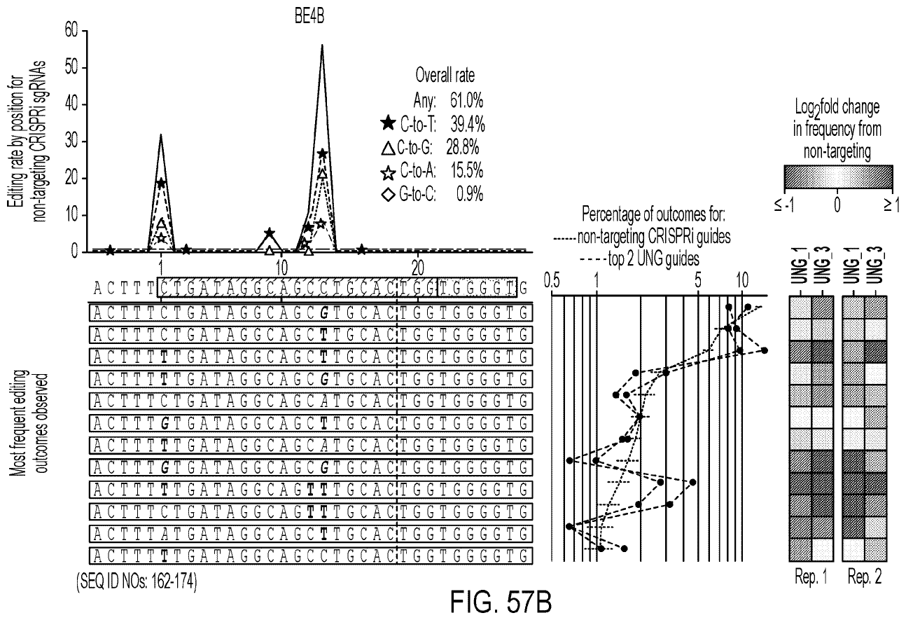

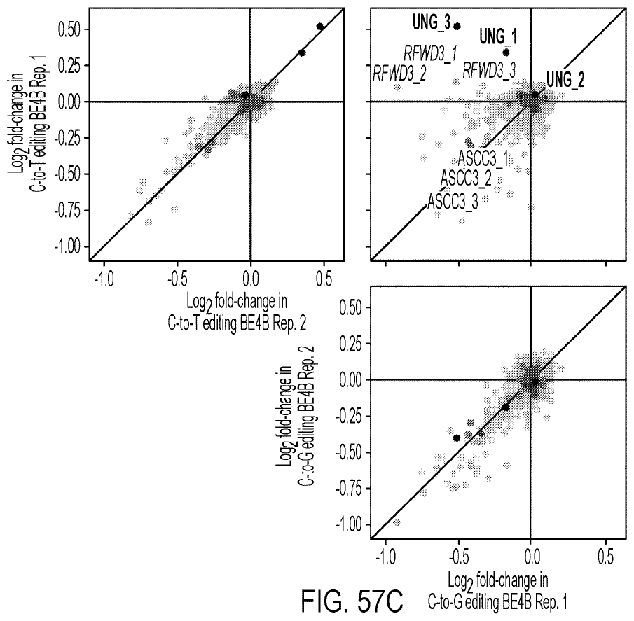

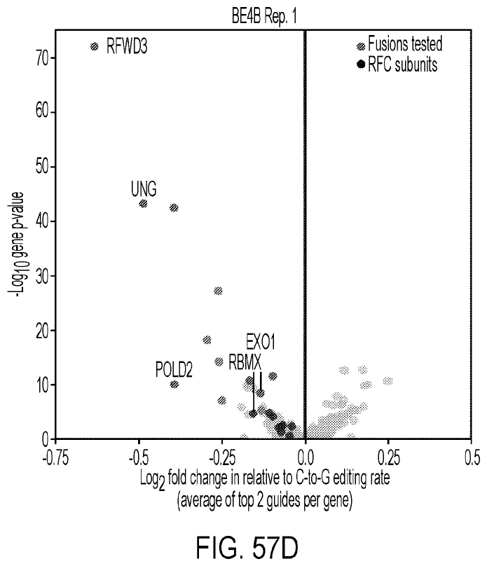

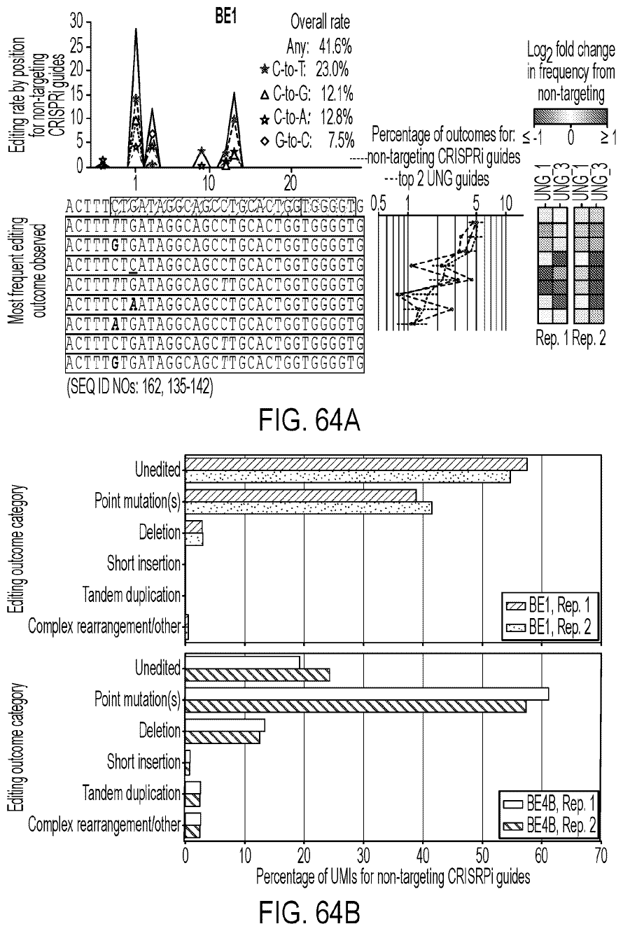

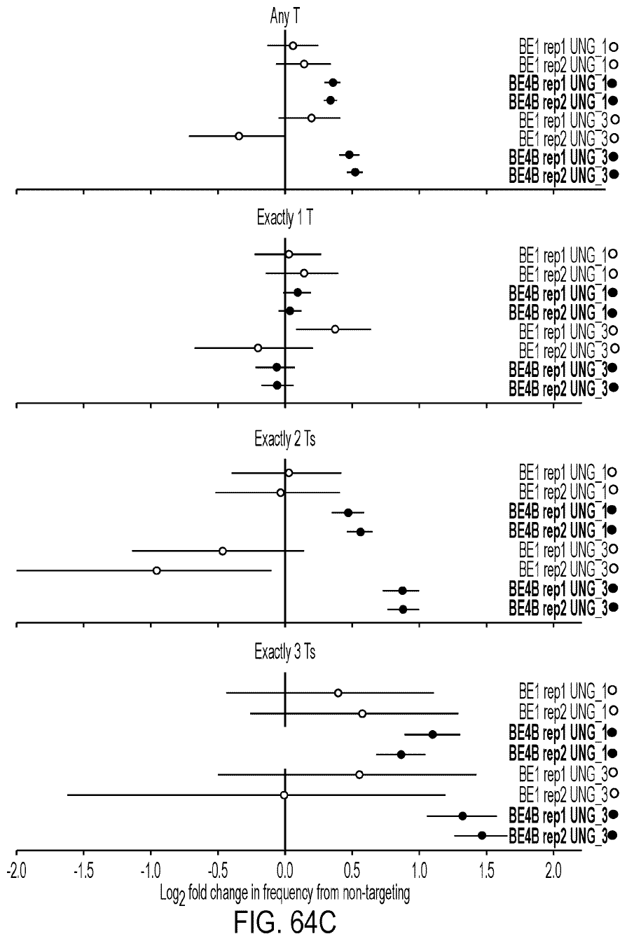

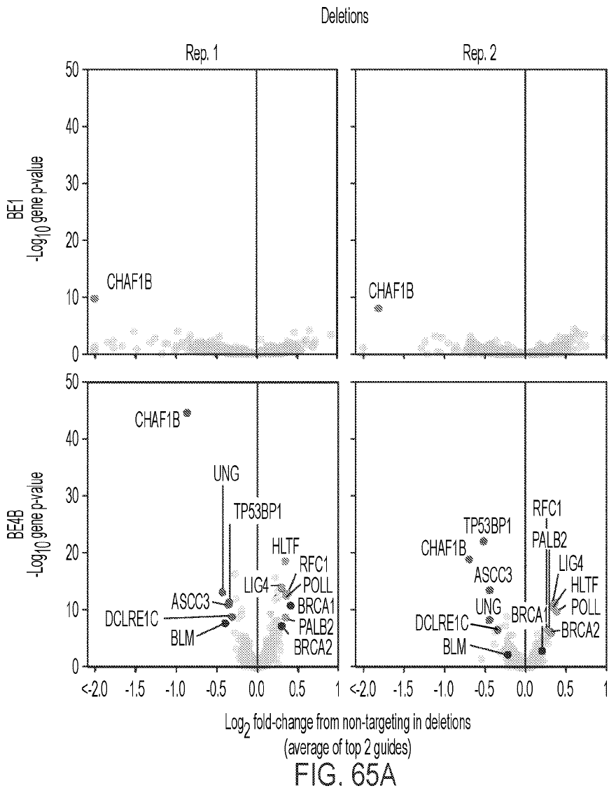

Using a recently developed screening platform capable of reading out DNA repair outcomes by DNA sequencing (FIGS. 57A-57B, FIG. 64A) (see Hussmann et al., Mapping the Genetic Landscape of DNA Double-strand Break Repair. Cell (2021) 184(22), 5653-5669.e25, which is herein incorporated by reference), the impact of knockdown of each of 476 genes, a set enriched for regulators of DNA repair, on the activity of BE1 (deaminase-dCas9) and BE4B (AC) editors was investigated. Briefly, an sgRNA library (1,513 gene-targeting sgRNAs and 60 non-targeting controls) was transduced into HeLa cells stably expressing the CRISPRi effector dSpCas9-KRAB21. After allowing 5 days for gene knockdown, the cells were transfected with plasmids encoding SaCas9-based CBEs (either SaCas9-BE1 or SaCas9-BE4B) and an SaCas9 sgRNA that targets a sequence adjacent to the genomically integrated SpCas9 sgRNA sequences. Notably, SaCas9-based CBEs were used to avoid guide RNA exchange between the base editors and CRISPRi machinery. A key aspect of this approach was that the proximity of the target site and CRISPRi sgRNA enabled these features to be read out together by paired-end DNA sequencing, thus linking editing outcomes to CRISPRi perturbation identities (FIG. 57A). To prepare samples for sequencing, genomic DNA from treated cells was isolated, unique molecular identifiers (UMIs) were affixed to DNA fragments containing both the sgRNA expression cassettes and edited target sites, and the linked sgRNA, target sites, and UMI sequences were sequenced. Comparing frequencies of editing outcomes from each CRISPRi sgRNA with those from non-targeting sgRNAs (FIG. 57B, FIG. 64A) then identified genes that promote or suppress various editing outcomes.

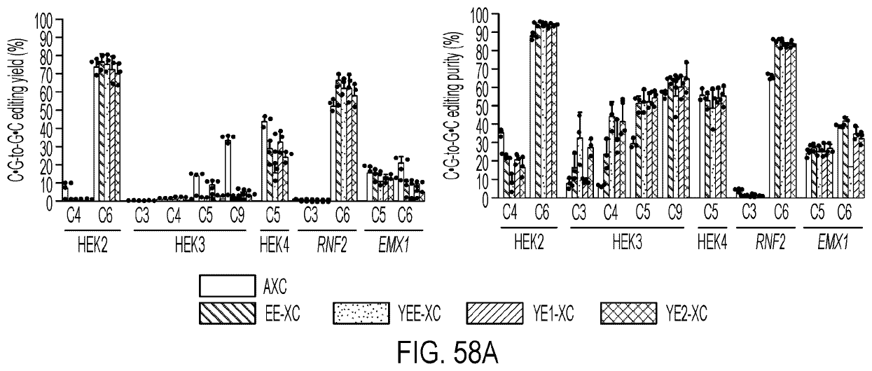

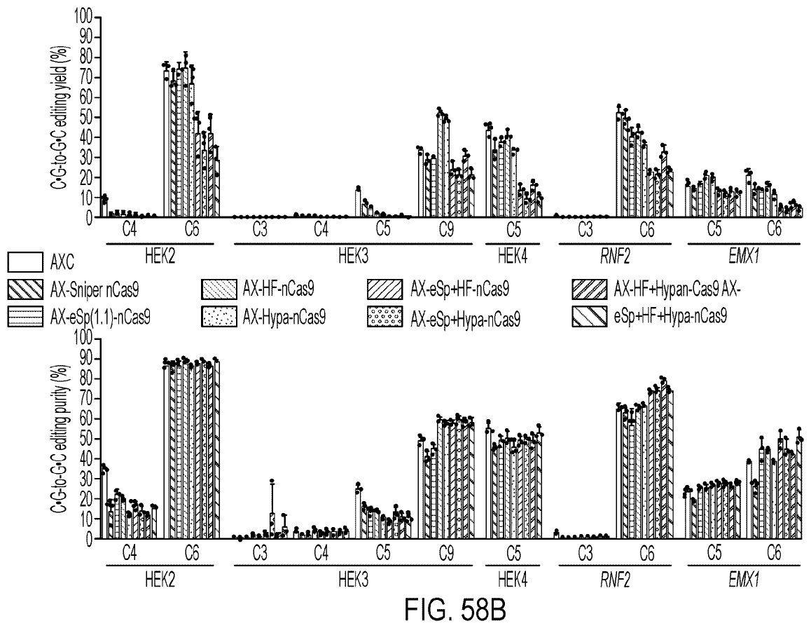

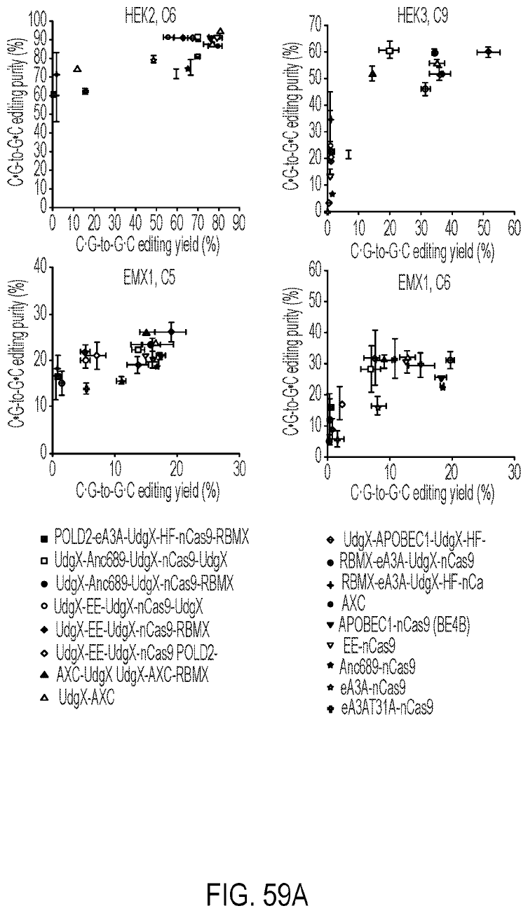

Consistent baseline activity of BE1 and BE4B in the screens enabled quantitation of editing differences driven by CRISPRi sgRNAs (FIGS. 57A-57D, FIGS. 64A-64C, FIGS. 65A-65E). To evaluate differences in point mutations, the effects of all CRISPRi sgRNAs on the frequencies of two major categories were calculated: outcomes containing any C•G-to-T•A point mutation and outcomes containing any C•G-to-G•C point mutation (FIG. 57C). For both classes, the effects of individual CRISPRi sgRNAs were consistent between replicates (FIG. 57C, upper left and lower right panels). Comparison between classes though revealed that some CRISPRi sgRNAs showed different effects on C•G-to-T•A versus C•G-to-G•C outcomes (FIG. 57C, upper right panel), indicating that specific genes influence partitioning between these outcomes. In the BE4B screen, the clearest differential effects resulted from sgRNAs targeting UNG (FIGS. 57B-57C). Consistent with the effects of UGI fusions and UNG loss^{2,11}, UNG knockdown increased frequencies of C•G-to-T•A editing while decreasing frequencies of C•G-to-G•C editing. Notably, the effects of UNG repression on BE1 editing were not as significant or straightforward (FIG. 58A, FIG. 58C), perhaps reflecting differences in how nicked versus unnicked target substrates are processed (FIG. 57B, FIG. 58A).

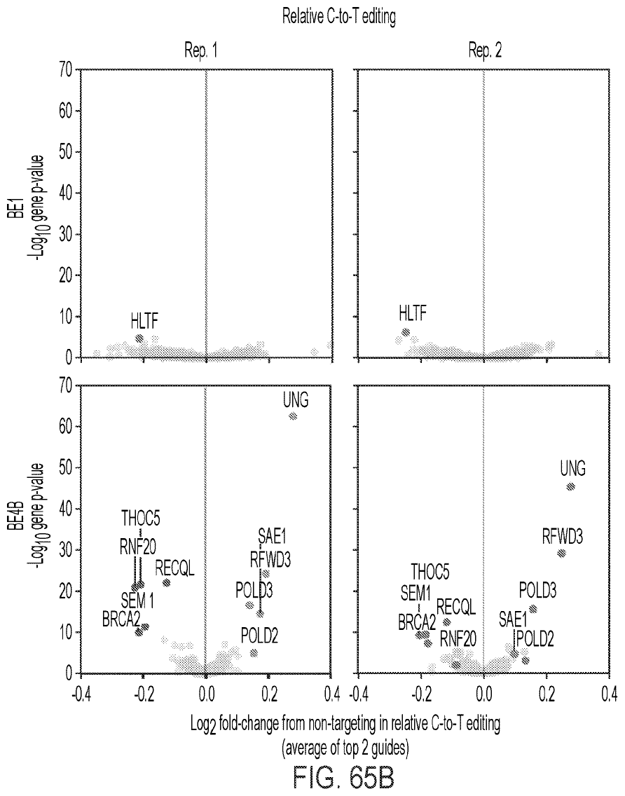

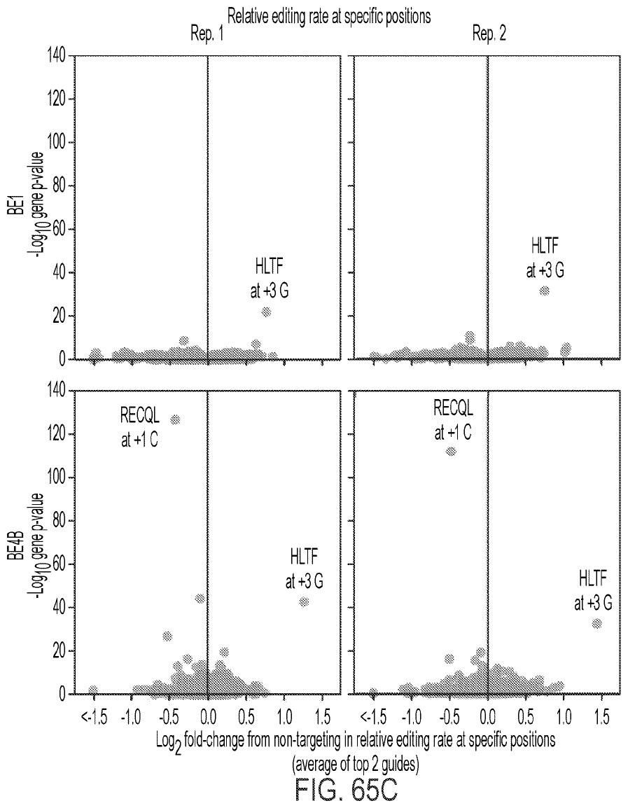

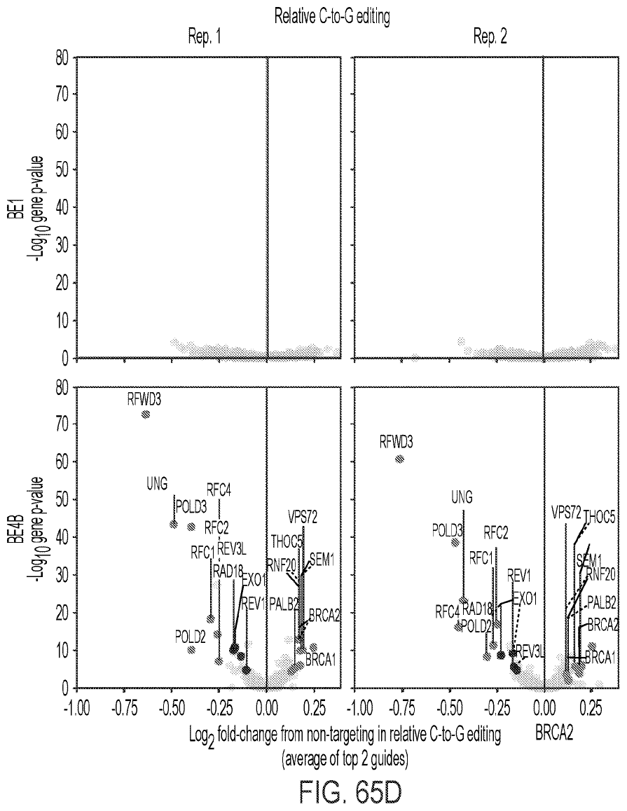

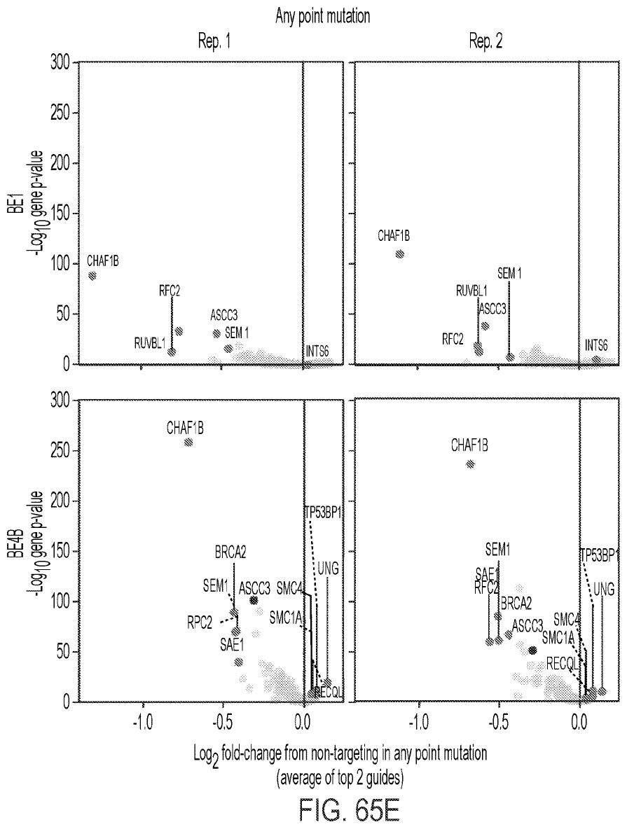

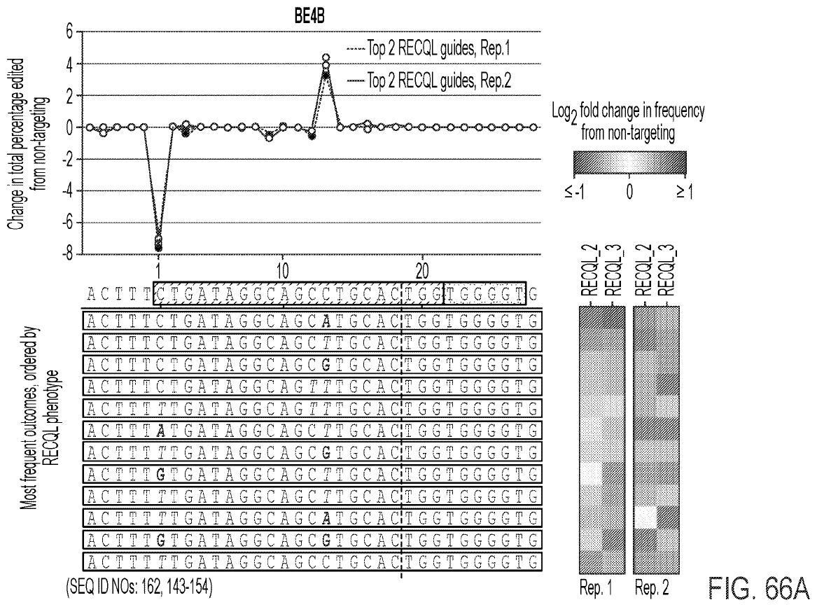

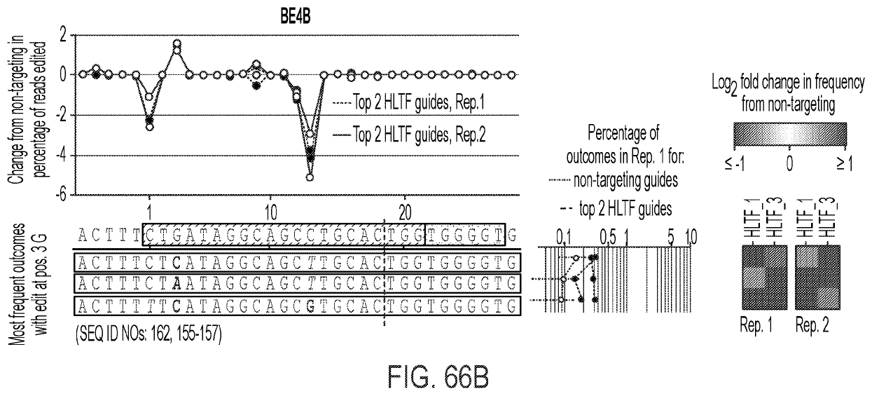

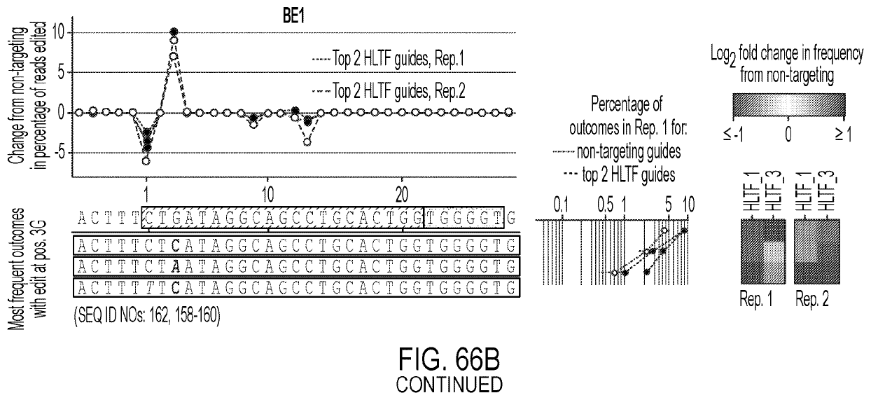

One advantage to screening with sequencing-based readouts was that changes to a diverse range of editing products could be detected. For example, it was also observed that CRISPRi-mediated depletion of double-strand breaks (DSB) repair genes affect the frequency of rare indels caused by base editing, though these pathway-phenotype relationships were not always straightforward (FIG. 65A). Indeed, while knockdown of HDR factors BRCA1, BRCA2, and PALB2 increased AC-generated deletions, depletion of the HDR gene BLM decreased them. Interestingly, depletion of BRCA2 was also among the strongest reducers of C•G-to-T•A editing outcomes (FIG. 65B). Genes that affect the base editing window were also identified (FIG. 65C, FIGS. 66A-66B).

Using screening data, genes that control the base editing activity window were identified. For each CRISPRi sgRNA, the fraction of all edited reads that included a point mutation were calculated at each position in or near the target sequence. Then, genes that significantly changed the relative editing frequency at any nucleotide position compared to non-targeting CRISPRi sgRNA controls were identified (FIG. 65C). Intriguingly, two helicase genes, RECQL and HLTF, emerged from this analysis. Repression of RECQL selectively reduced editing at the PAM-distal C in position +1 of the target sequence, where the SaCas9 NNGRRT (SEQ ID NO: 223) PAM is positions 22-27 (FIGS. 66A-66B), while repression of HLTF specifically increased editing at the G in position +3 (FIGS. 66A-66B). Together, these observations suggest that cellular helicases can influence the location of base editing activity within a target sequence, potentially by increasing the accessibility of cytosines at position +1 in the case of RECQL, or by reducing accessibility of the C opposite the position +3 G in the case of HLTF.

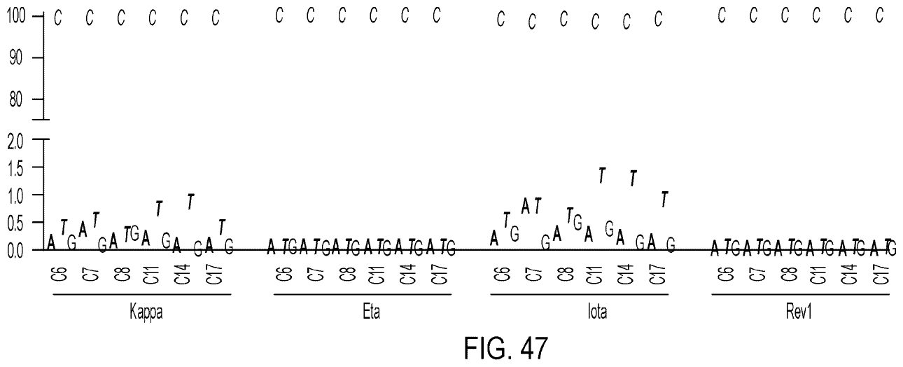

To identify genes that specifically promoted C•G-to-G•C editing, the relative fraction of outcomes containing any C•G-to-G•C edit among outcomes containing any point mutation for each CRISPRi sgRNA were calculated (FIG. 47D, FIG. 65D). The gene whose knockdown most significantly reduced the C•G-to-G•C editing fraction compared to non-targeting sgRNAs was RFWD3, an E3 ligase with multiple roles in DNA repair recently identified as required for successful translesion synthesis across a variety of genomic lesions²². Other hits included UNG; multiple subunits of the replicative polymerase POLD and replicative clamp loader RFC; EXO1; translesion polymerases REV1 and REV3L; and RAD18, an E3 ubiquitin ligase involved in translesion synthesis.

The different phenotypes for REV1 knockdown versus the individual knockout cell line may arise from compensatory mechanisms that could alter DNA repair outcomes in cells lacking REV1. Genes whose knockdown reduced frequencies of both C•G-to-T•A and C•G-to-G•C base editing for both BE1 and BE4B were also identified (FIG. 65E), including ASCC3, which may act by affecting accessibility of the target locus, a known determinant of base editing efficiency^{2,3,8}. Together, these screen results suggest important roles for DNA replication processes, especially translesion synthesis, in modulating C•G-to-G•C base editing outcomes.

### CBE Fusion Proteins can Alter C•G-to-G•C Transversion Outcomes

To further advance the development of CGBEs, new CGBE candidates were generated by fusing AXC, the prototype CGBE described above, to proteins nominated by the CRISPRi screens. These included those encoded by genes that reduced C•G-to-G•C editing following knockdown, including DDX1, EXO1, POLD1, POLD2, POLD3, RAD18, RBMX, REV1, RFWD3, and TIMELESS, and several additional genes involved in DNA polymerization, some of which also affected editing outcomes in the CRISPRi screen (PCNA, POLH, POLK, UBE2I, and UBE2T).

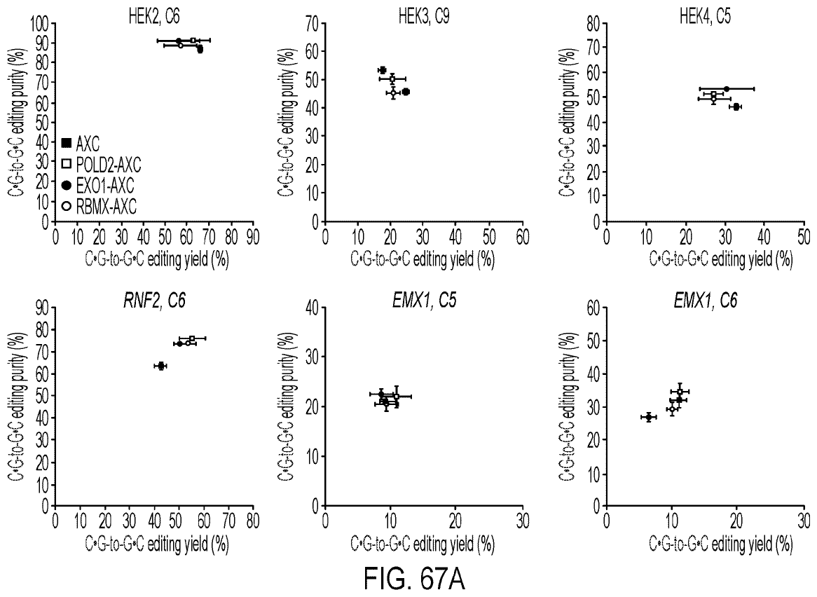

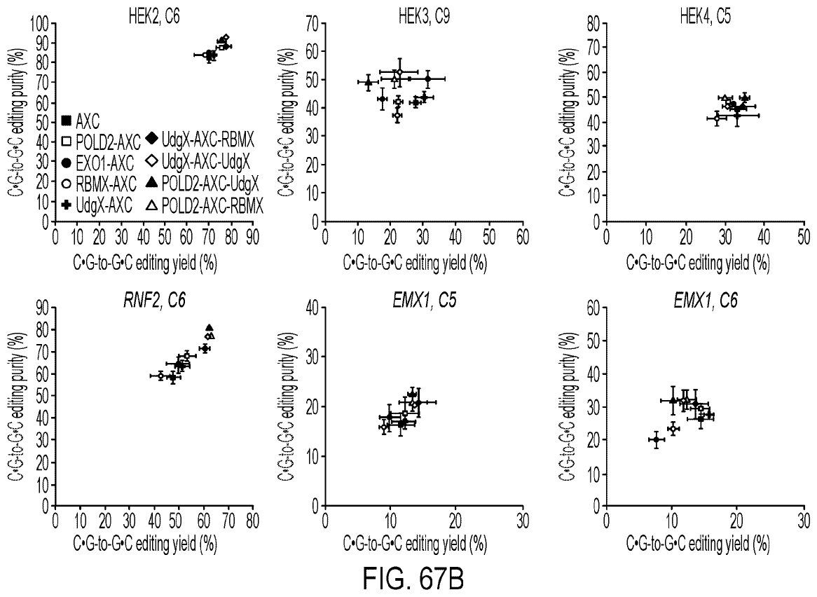

Each of these proteins were fused to the N- or C-terminus of AXC to assess their effect on C•G-to-G•C editing efficiency or purity and assessed their editing performance at five genomic loci in HEK293T cells. Three proteins increased C•G-to-G•C editing purity when fused to the N-terminus of AXC (FIG. 67A): DNA polymerase D2 (POLD2), exonuclease 1 (EXO1), and RNA binding motif protein X-linked (RBMX). Editing improvements for fused constructs varied by site. The most pronounced effects were observed at the RNF2 locus, where editing purity significantly improved from 54±1.4% with AXC to 73±0.4% with RBMX-AXC, 74±1.4% for EXO1-AXC, and 77±0.8% for POLD2-AXC (p<0.001, Student's two-sided t-test). Marginal improvements in purity were also observed at HEK site 2, HEK site 3, and HEK site 4 loci. A significant increase in editing yield was also observed at RNF2, from 43±2.4% with AXC to 50±5.2% with RBMX-AXC, 53±3.6% with EXO1-AXC, and 55±5.5% for POLD2-AXC (p<0.05, Student's two-sided t-test). C-terminal fusions typically did not perform as well as N-terminal fusions.

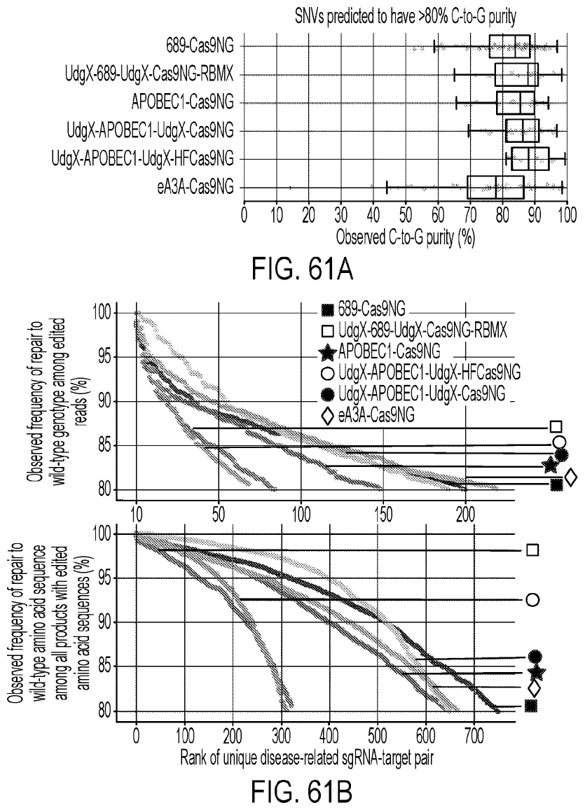

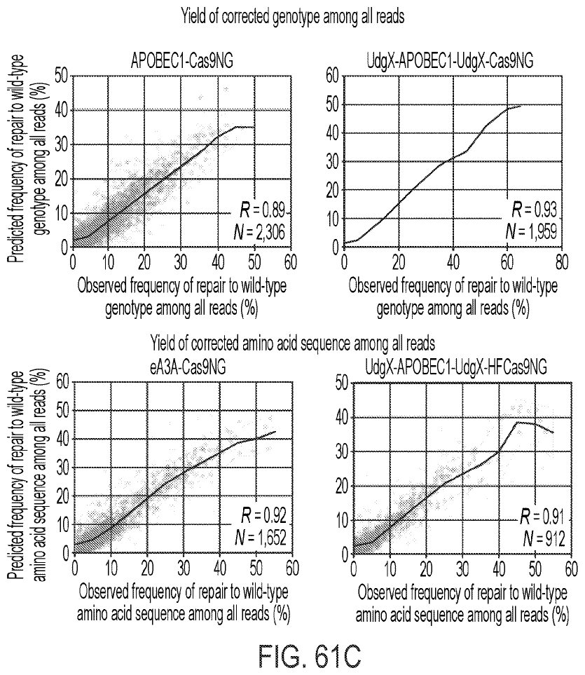

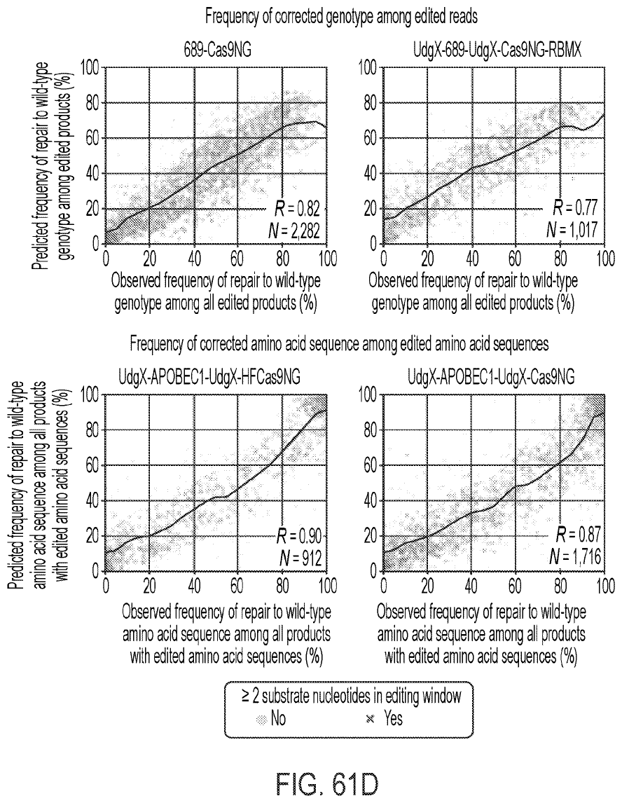

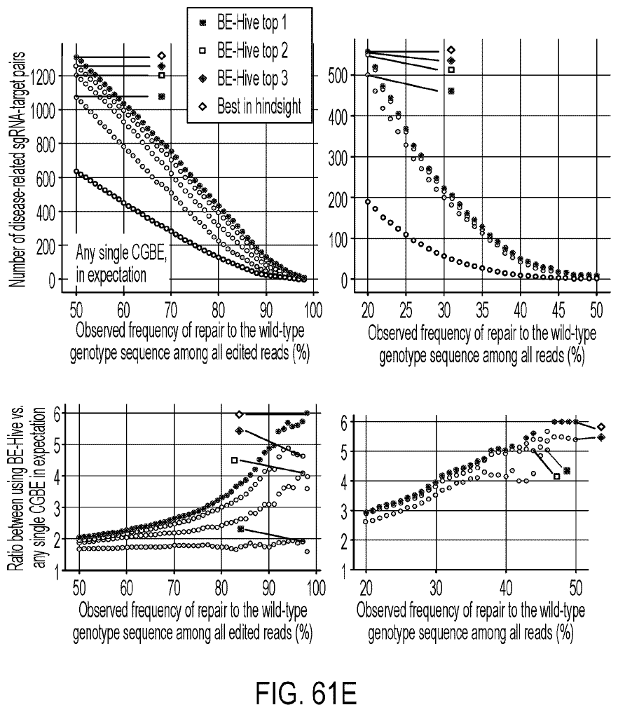

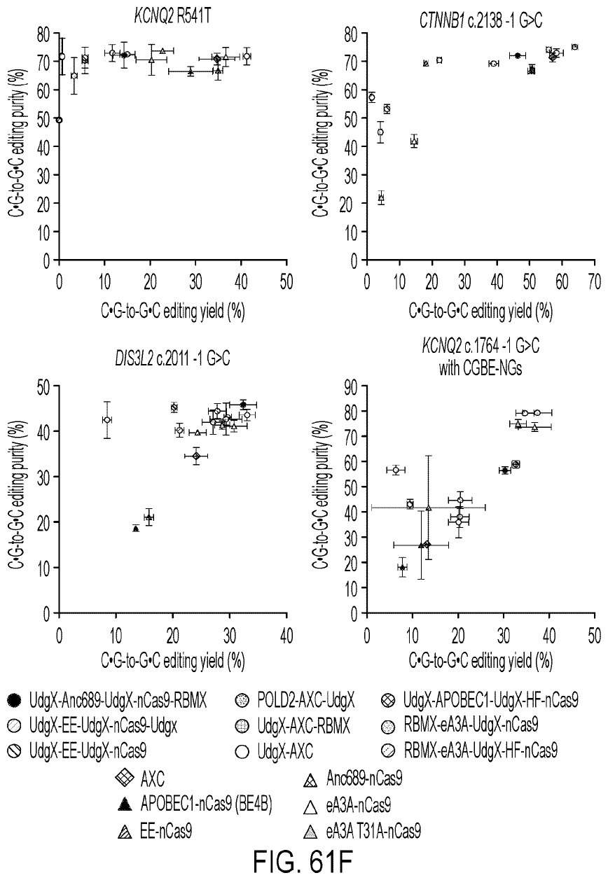

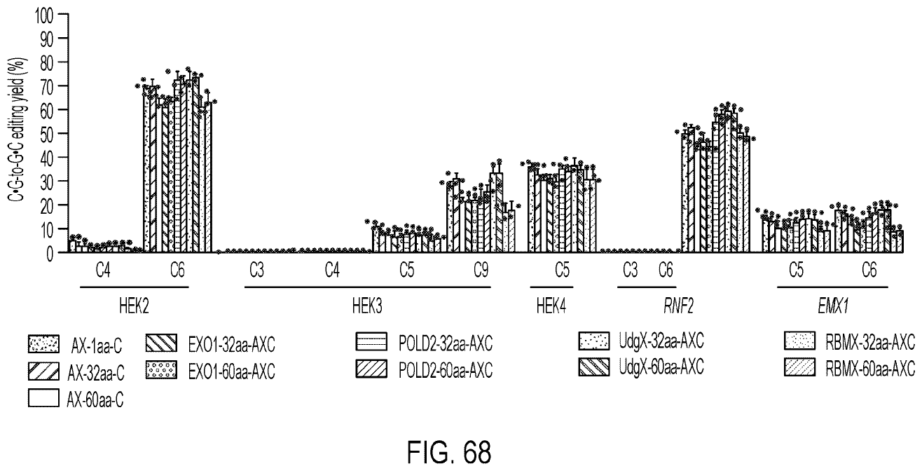

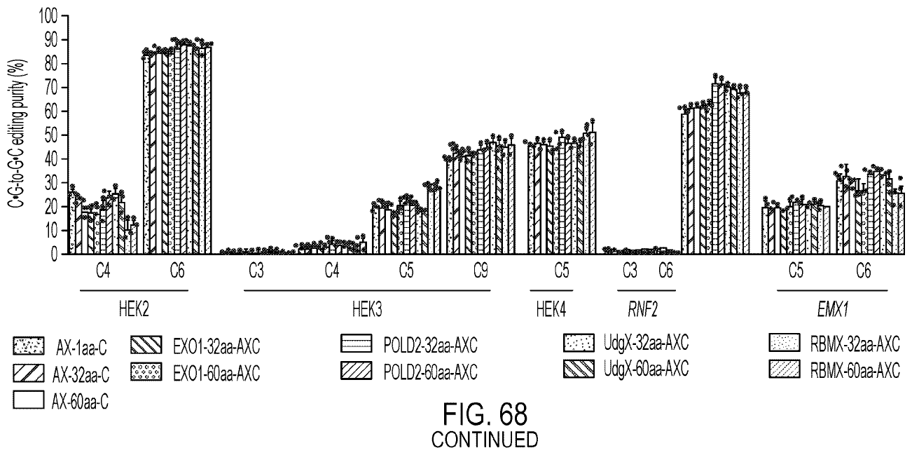

Encouraged by these improvements, additional candidate CGBEs were developed containing RBMX, EX01, POLD2, and UdgX as fusions to AXC. Single and dual pairwise fusion architectures were compared for these components, testing N- and C-terminal dual fusions as well as tandem N terminal fusions (N-, N-) using 32-residue linkers identified in a linker-testing experiment for these constructs (FIG. 68). From a total of 28 single- and dual-fusion proteins tested, the four dual fusion architectures POLD2-deaminase-UdgX-nCas9-RBMX, POLD2-deaminase-UdgX-nCas9-UdgX, UdgX-deaminase-UdgX-nCas9-UdgX, and UdgX-deaminase-UdgX-nCas9-RBMX further increased C•G-to-G•C editor yield and purity at some sites (on average, by +10% and +13%, respectively) compared to single fusion architectures across nine cytosines in five genomic loci (FIG. 61B).

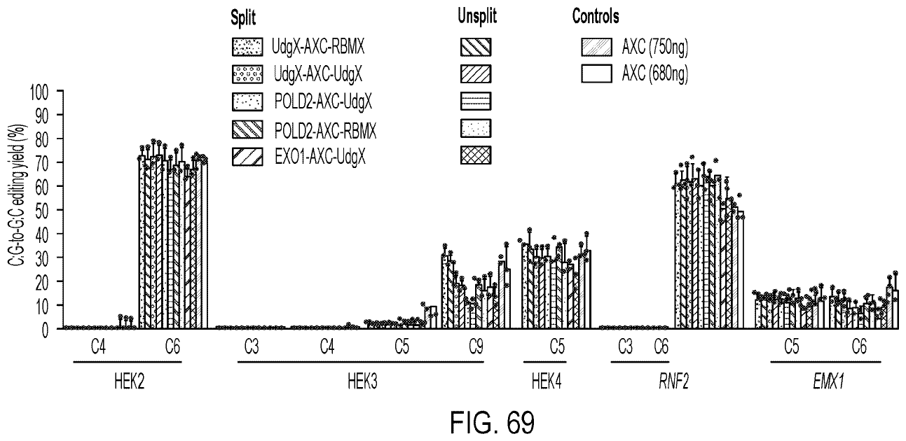

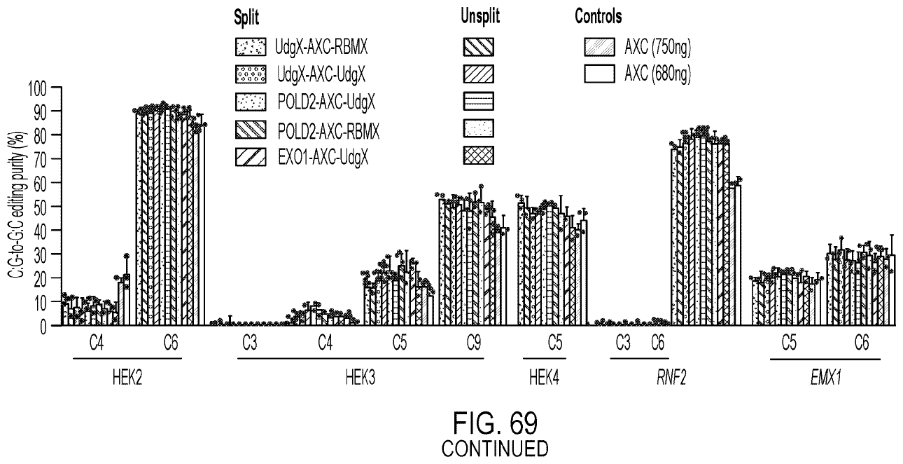

Collectively, these results indicate that CGBEs, including fusions to proteins identified in the CRISPRi screen, can affect C•G-to-G•C editing outcomes in a site-dependent manner. Some base editing applications may prioritize protein size over other base editing characteristics. Therefore, the use of trans-splicing split-inteins was explored as a means to reduce the size of large CGBEs into two smaller protein components²³, and observed no changes in editing outcomes of split-CGBEs compared to their full-length counterparts (FIG. 69). When necessary, these split CGBE variants may support favorable cytosine transversion outcomes without requiring the expression of full-length proteins.

### Base Editor Deaminase and Cas9 Domains Bias Repair Outcomes

Next, different deaminase domains were studied to determine how they affect C•G-to-G•C editing in the AXC architecture. Since the base editing window may influence cytosine transversion outcomes^{2,11,12} a panel of catalytically impaired deaminases that support different CBE editing windows²⁴ were examined, and an increase in C•G-to-G•C editing purity was observed at three of five tested loci (FIG. 58A). The APOBEC1 R126E R132E (EE)²⁴ deaminase showed the greatest improvement, averaging 1.2-fold higher product purity at HEK site 2, HEK site 3, and RNF2. Editing yield with these deaminase alternatives varied by locus. Similar or reduced editing yield compared to AXC was observed at four out of five loci—likely due to the lower catalytic activity of these deaminases, though reduced yield did not correlate with altered C•G-to-G•C purity. Editing yield by EE-AXC at the RNF2 locus significantly improved (AXC=52±3.2% vs. EE-AXC=66±3.5%, p=0.007, Student's two-sided t-test).

It was also hypothesized that changes to the Cas9 binding domain of CGBEs could alter editing windows and C•G-to-G•C editing outcomes by altering the competition between Cas9 and repair machinery for access to the target locus. AXC editors that use Cas9 variants were assessed with different binding kinetics, including new variants with combinations of previously reported Cas9 mutations (FIG. 58B)²⁵-2⁸. AX-HF-nCas9 substantially improved C•G-to-G•C editing at the C9 position of the HEK site 3 locus, increasing yield (AXC=34±1.9% vs. AX-HF-nCas9=52±1.7%,) and purity (AXC=49±2.2% vs. AX-HF-nCas9=60±1.2%) (p<0.005 for both, Student's two-sided t-test) (FIG. 58B). AX-Hypa-nCas9 showed similar effects but AX-HF-nCas9 typically performed modestly better. These results suggest Cas protein binding parameters can affect C•G-to-G•C editing yield and purity of CGBEs at some target loci.

The balance of editing yield and purity among candidate CGBEs and the variability in these two measures across different loci suggests that different target sites will be best edited by different CGBEs. Therefore, a suite of CGBEs with different kinetics and substrate preferences would likely enable efficient and high-purity C•G-to-G•C editing across a broader range of diverse target sequences than could be achieved by any single CGBE variant alone. Combining deaminase, Cas9 domain, and DNA repair fusion proteins into new CGBEs

The above findings from varying protein fusions, deaminases, and Cas domains were integrated into improved CGBEs. The four most promising dual-fusion AXC editors (POLD2-AXC-RBMX, POLD2-AXC-UdgX, UdgX-AXC-RBMX, and UdgX-AXC-UdgX), four single-fusion AXC editors (POLD2-AXC, RBMX-AXC, EXO1-AXC, and UdgX-AXC), AXCs with deaminase variants of those same editors, and direct deaminase-nCas9 CGBEs without additional fusion proteins were evaluated. The five cytidine deaminases tested in these 10 CGBE architectures included rAPOBEC1, EE, Anc689 (ancestrally-reconstructed rAPOBEC1 node 689²⁹), evolved APOBEC3A (A3A), and eA3A-T31A¹². See International Publication Nos. WO 2019/023680, published Jan. 31, 2019; WO 2019/226953, published Nov. 28, 2019; Kim, Y. B. et al. Nature Biotechnology (2017); and Gehrke et al. Nature Biotechnology (2018), each of which is incorporated by reference herein. In addition, both SpCas9 nickase and HF-Cas9 nickase variants were tested. In total, 95 candidate CGBEs were evaluated at eight genomic loci in HEK293T cells.

The editor architectures generated and evaluated are listed below. In each of these constructs, the 32 amino acid linker refers to the linker having the amino acid sequence set forth as SEQ ID NO: 108). The terminator may be any transcriptional terminator, such as an SV40 or bovine growth hormone polyadenylation (polyA) sequence: BE4B constructs

Promoter-BPNLS-[Deaminase]-32 amino acid linker-[Cas9 effector domain]-BPNLS-Terminator

### C-Terminal Glycosylase Constructs

Promoter-BPNLS-[Deaminase]-32 amino acid linker-[Cas9 effector domain]-SGGS-[Glycosylase variant]-BPNLS-Terminator

### Glycosylase Architecture Constructs

N-terminal: Promoter-BPNLS-[Glycosylase variant]-SGGS-[Deaminase]-32 amino acid linker-[Cas9 effector domain]-SGGS linker-BPNLS-Terminator

Internal: Promoter-BPNLS-[Deaminase]-32 amino acid linker-[Glycosylase variant]-32 amino acid linker-[Cas9 effector domain]-BPNLS-Terminator

C-terminal: Promoter-BPNLS-[Deaminase]-32 amino acid linker-[Cas9 effector domain]-SGGS linker-[Glycosylase variant]-BPNLS-Terminator

### Single Fusion Screen Hit Architecture Constructs

N-terminal: Promoter-BPNLS-[Screen Hit]-32 amino acid linker-[Deaminase]-32 amino acid linker-UdgX-[Cas9 effector domain]-BPNLS-Terminator

C-terminal: Promoter-BPNLS-[Deaminase]-32 amino acid linker-UdgX-[Cas9 effector domain] 32 amino acid linker-[Screen Hit]-BPNLS-Terminator

### Dual Fusion Screen Hit Architecture Constructs

Dual N-, N-terminal: Promoter-BPNLS-[Screen Hit]-32 amino acid linker-[Screen Hit]-32 amino acid linker-[Deaminase]-32 amino acid linker-UdgX-[Cas9 effector domain]-BPNLS-Terminator

N- and C-terminal: Promoter-BPNLS-[Screen Hit]-32 amino acid linker-[Deaminase]-32 amino acid linker-UdgX-[Cas9 effector domain] 32 amino acid linker-[Screen Hit]-BPNLS-Terminator.

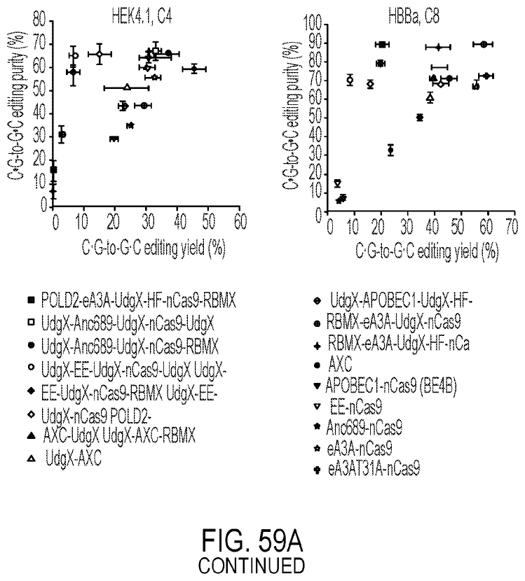

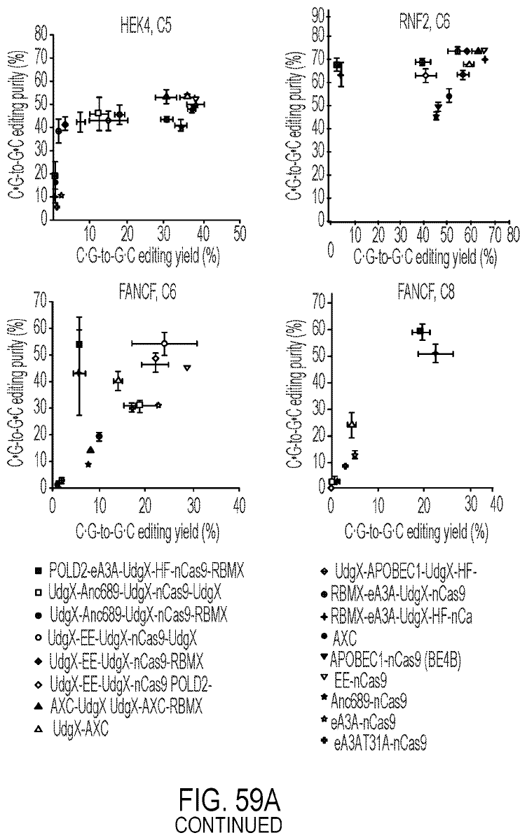

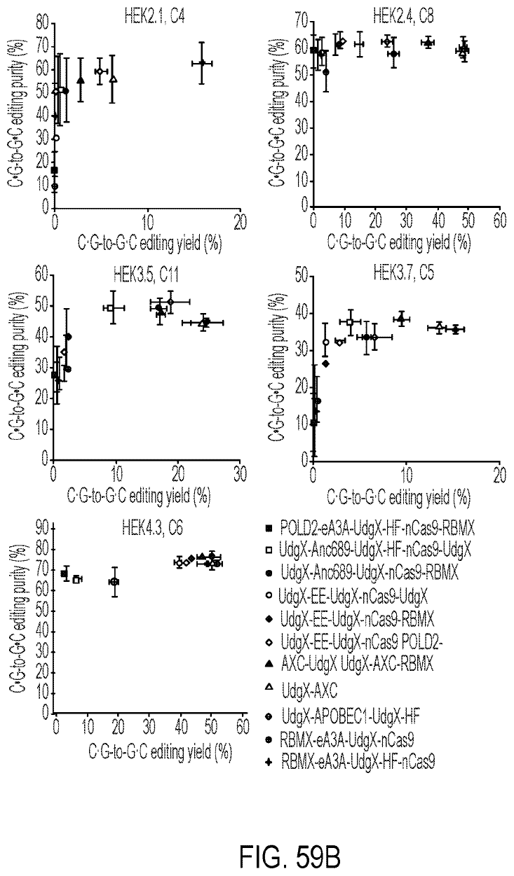

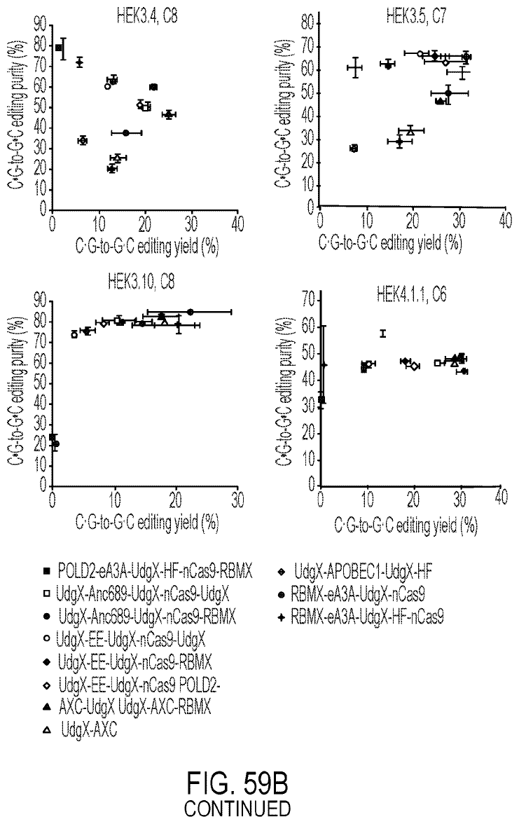

No single CGBE outperformed all other candidates at all sites (FIG. 59A). To identify a set of the most promising CGBEs, 32 editors that demonstrated improved C•G-to-G•C editing outcomes at some sites were selected for testing at eight additional genomic loci (FIG. 59B). These data were used to identify ten CGBEs with high purity, yield, and maximally distinct activities at different endogenous loci using quadratic programming and hierarchical clustering (Methods): Anc689-nCas9, UdgX-Anc689-UdgX-nCas9-RBMX, eA3A-nCas9, RBMX-eA3A-UdgX-HF-nCas9, RBMX-eA3A-UdgX-nCas9, EE-nCas9, UdgX-EE-UdgX-nCas9-UdgX, APOBEC1-nCas9, UdgX-APOBEC1-UdgX-HF-nCas9, and POLD2-APOBEC1-UdgX-nCas9-UdgX.

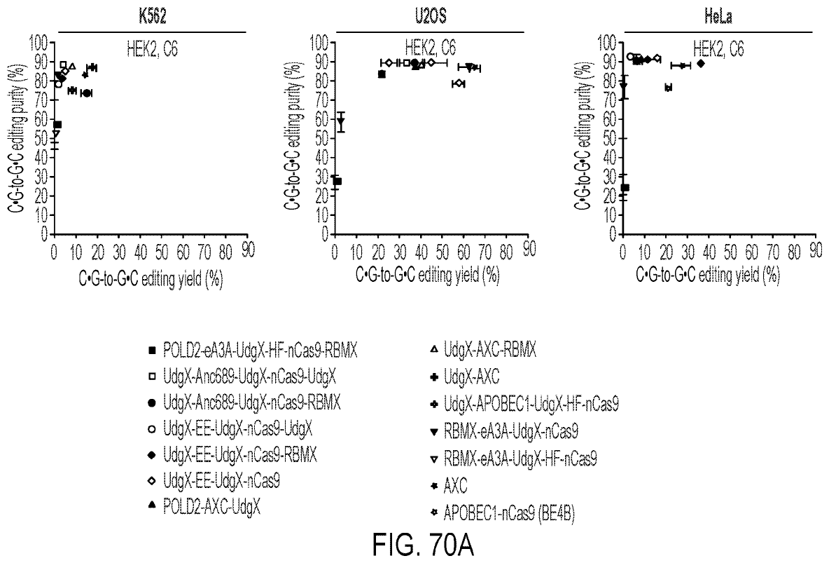

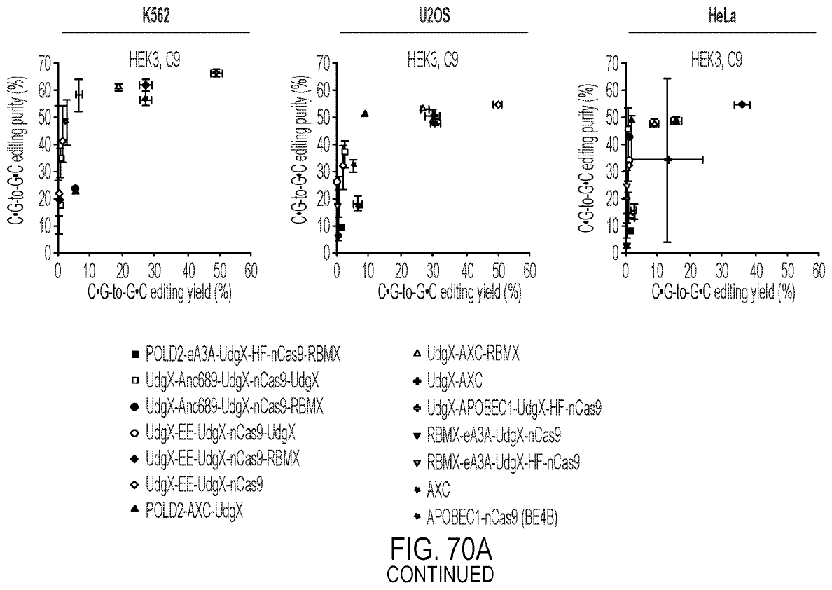

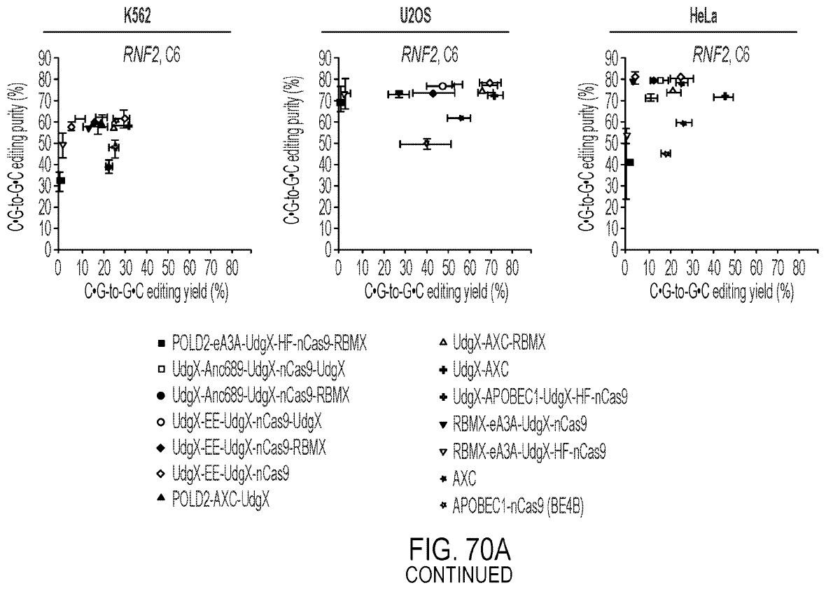

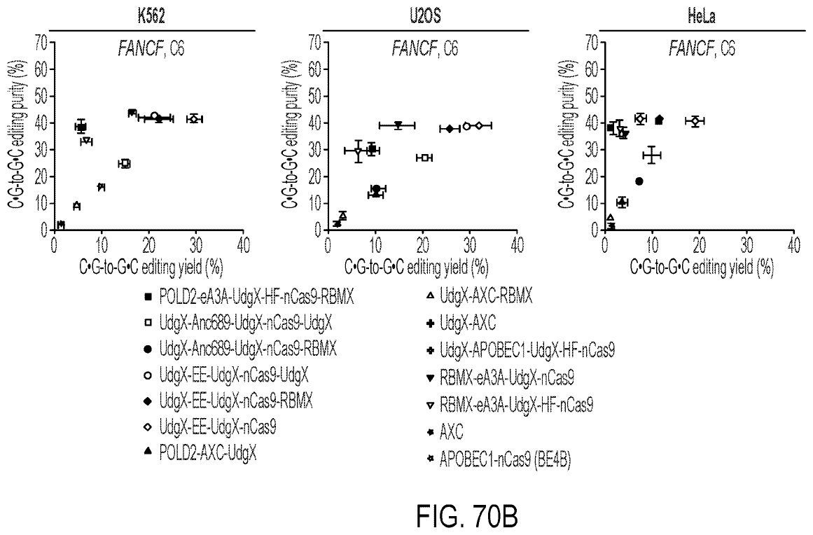

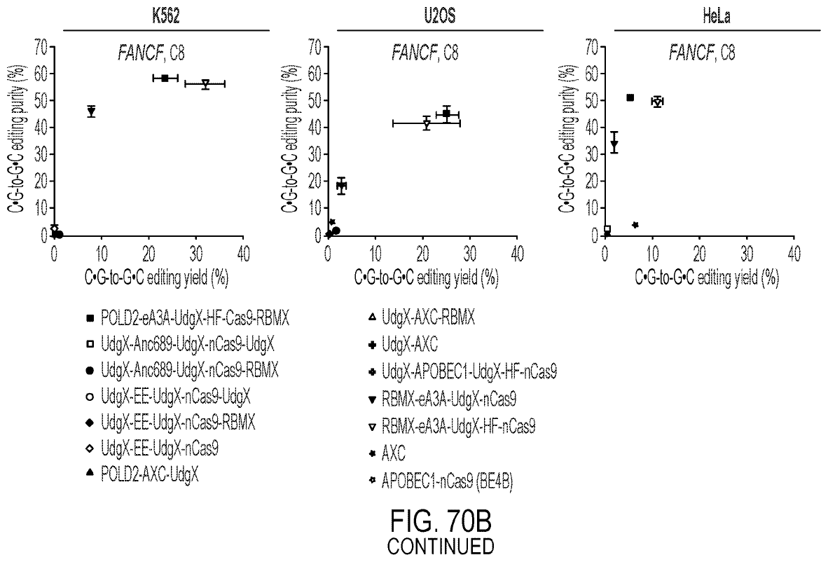

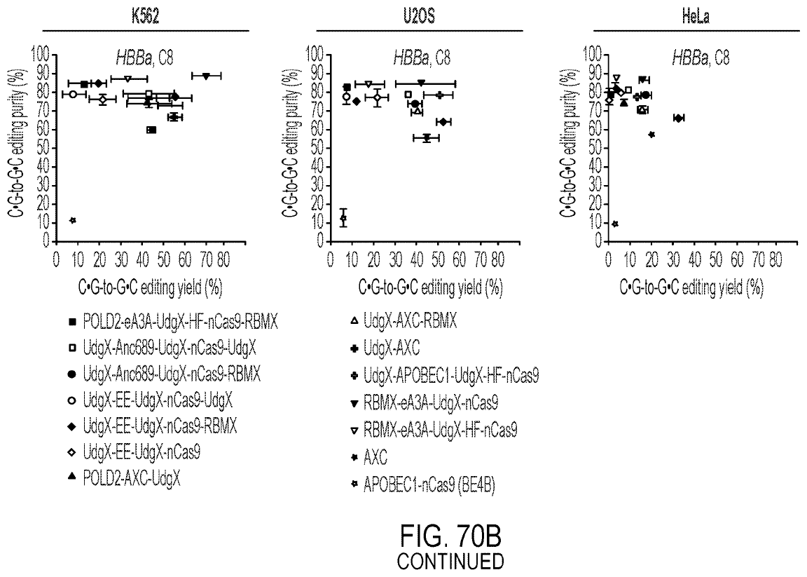

To test how this set of CGBEs performed in human cell lines other than HEK293T cells, the ability of each of these CGBEs to edit five target genomic sites in K562, U2OS, and HeLa was assayed (FIG. 70A-70B). It was observed that while CGBE outcomes vary modestly by cell type, the top-performing CGBE variants for each tested site were generally the same in all three additional cell lines. These results indicate that deaminase, Cas protein, and DNA repair protein variants can improve C•G-to-G•C editing in across different cell types.

### Target Library Characterization of CGBEs

It was observed that different target loci were best edited by different CGBEs, indicating that diverse CGBE sequence preferences may be strong determinants of C•G-to-G•C editing efficiency and purity. Previously, high-throughput analysis of base editing outcomes at thousands of genomically integrated target sequences was used to better understand CBE and ABE sequence-activity relationships, and these data were used to train machine learning models that facilitate the selection of target sequences amenable to C•G-to-G•C conversion by CBEs¹². It was envisioned that comprehensive characterization of the top ten promising and diverse CGBEs could similarly aid in the selection of targets amenable to efficient and high-purity C•G-to-G•C editing by specific CGBEs.

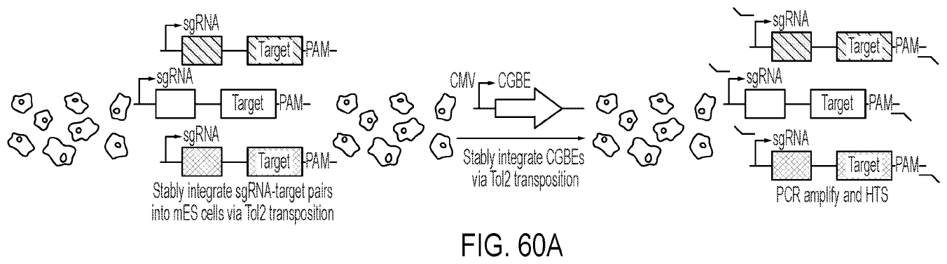

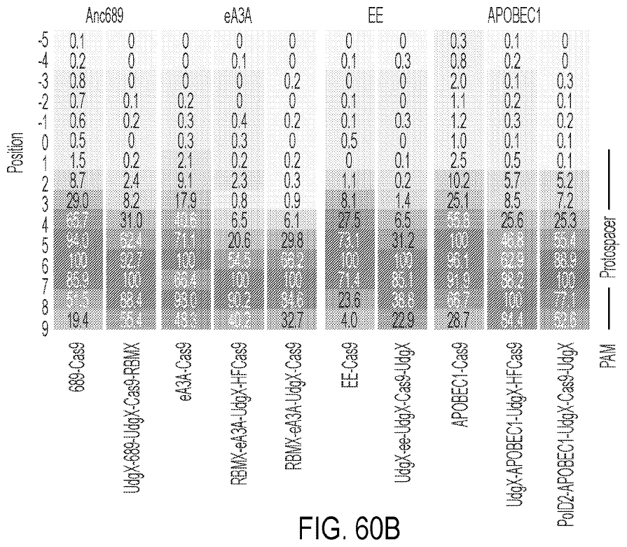

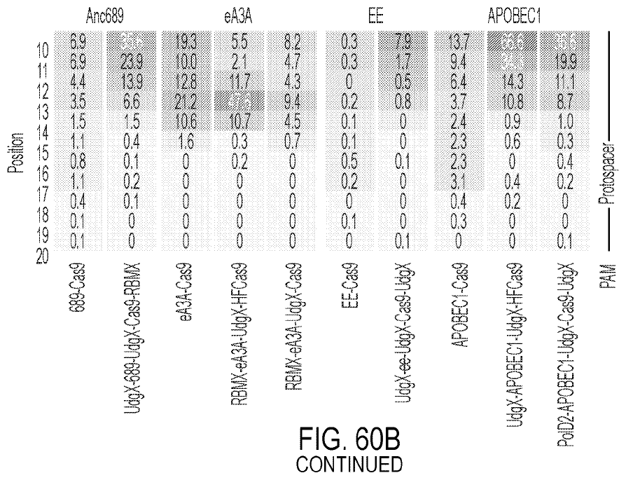

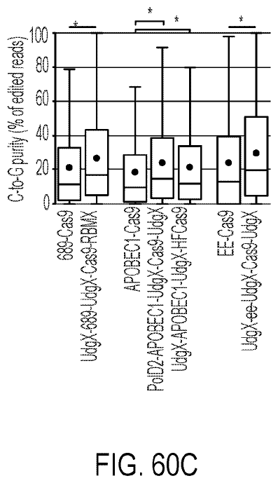

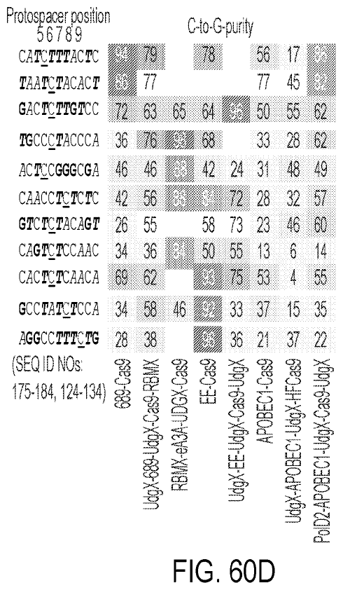

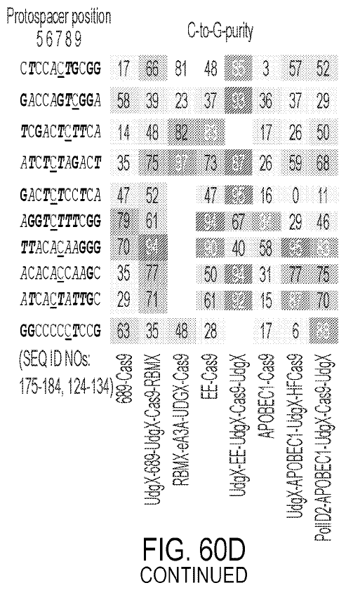

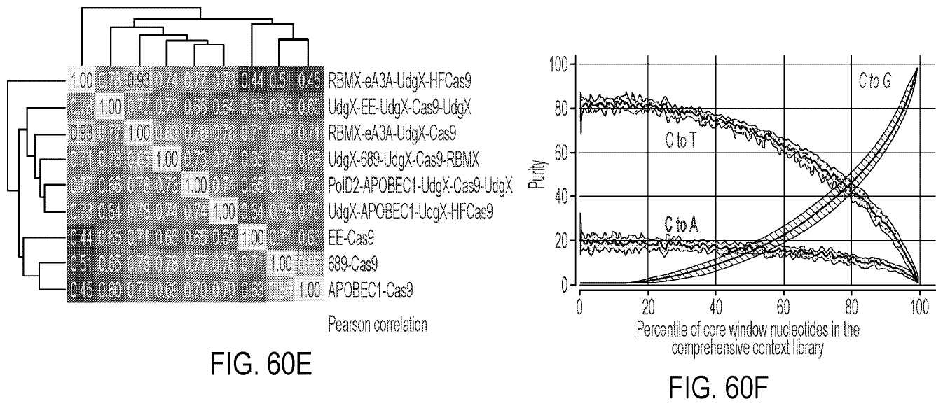

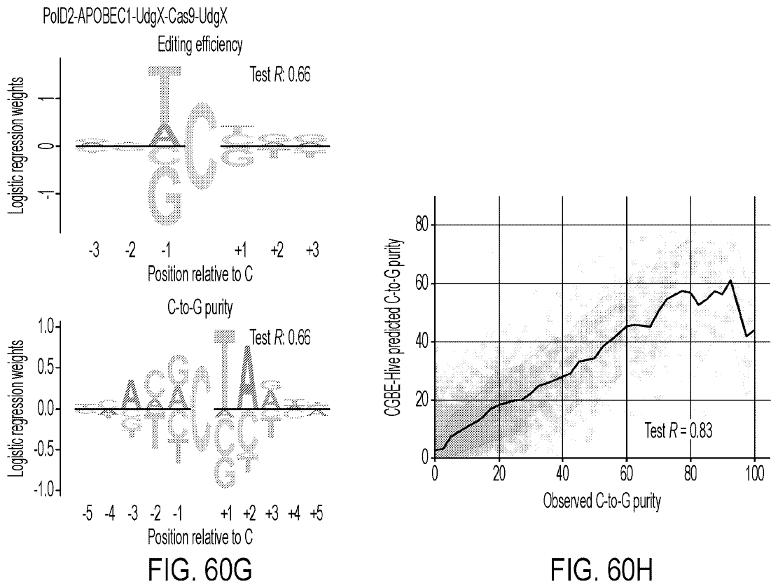

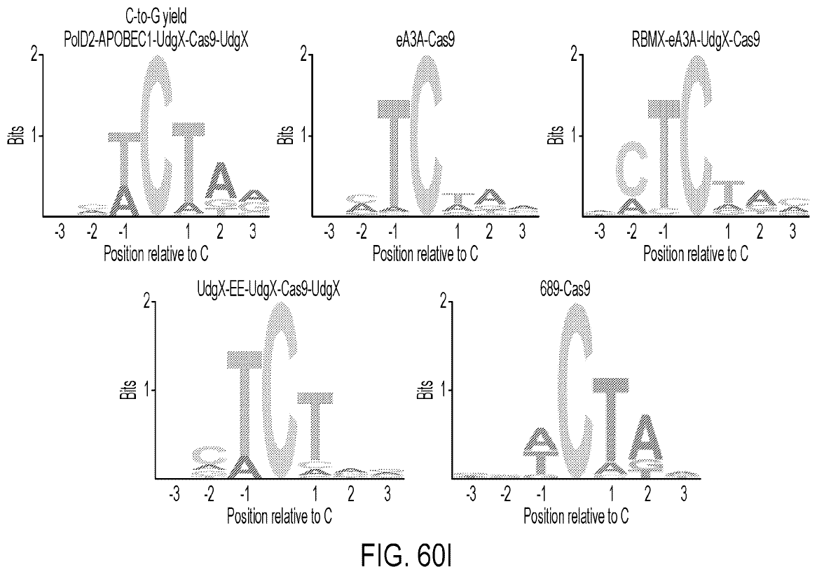

Each of the ten CGBEs were characterized using a high-throughput genome-integrated library assay of 10,638 matched sgRNA and target pairs in mESCs, previously referred to as the “comprehensive context library”¹². The target sequences in this library cover all possible sequence contexts surrounding the edited C•G with minimal sequence bias (FIG. 60A, Methods). To detect editing outcomes with high sensitivity, an average coverage of ≥300× per library member was maintained throughout the course of the experiment and an average sequencing depth of ≥4,000× per target. Two biological replicates were collected per CGBE characterization experiment. It was previously validated that the library assay data has strong consistency between biological replicates and is concordant with data from base editing endogenous genomic loci^{12,30}.

The resulting library data was used to quantify editing windows and product purities for each CGBE (FIG. 60B, Methods). CGBE editing activity was generally centered around protospacer position 6 with editing window widths ranging from 3 nt (EE-nCas9; positions 5-7) to 8 nt (UdgX-APOBEC1-UdgX-HF-nCas9 nickase; positions 4-11). The editing windows of CGBEs with additional components beyond Cas and deaminase domains were shifted by up to 3 nt compared to direct deaminase-Cas fusions, indicating that CGBE protein fusions can affect editing window size and position.

Engineered CGBE architectures showed significant improvements in C•G-to-G•C product purity compared to simple deaminase-nCas9 fusions. Across the 10,638 target sites in the comprehensive context library, the fusion CGBEs POLD2-APOBEC1-UdgX-nCas9-UdgX, UdgX-EE-UdgX-nCas9-UdgX, and UdgX-Anc689-UdgX-nCas9-RBMX showed 25% higher mean C•G-to-G•C purity than their corresponding deaminase-nCas9 counterparts within each editor's editing window (P<5.1×10-9; Welch's t-test) (FIG. 60C). A large variation in CGBE editing efficiency was observed, with mean efficiency ranging from 1.8% by UdgX-EE-UdgX-nCas9-UdgX to 23.0% by Anc689-nCas9 across the comprehensive context library within the same experimental batch. Notably, the protein fusion CGBEs exhibiting increased C•G-to-G•C purity also reduced editing yield by 1.4-to 1.6-fold on average.

C•G-to-G•C editing purity exceeded 90% for at least one of the tested CGBEs at 895 cytosines across the comprehensive context library. Some cytosines edited with purities as high as 90-100% by some CGBEs were edited with purity as low as 0-10% by other CGBEs, indicating that these CGBEs indeed offer complementary editing characteristics, and confirming that a panel of diverse CGBEs maximizes the utility of C•G-to-G•C base editing compared to using any single CGBE (FIG. 60D). CGBEs were clustered by C•G-to-G•C editing purity across the comprehensive context library and observed that engineered CGBEs did not cluster by deaminase (FIG. 60E), indicating that protein fusion engineering of CGBE architectures resulted in distinct sequence preferences governing C•G-to-G•C editing.

### Sequence Determinants and Machine Learning Modeling of CGBE Activity

C•G-to-G•C product purity of CGBEs varies substantially by sequence context (FIG. 5F). A 24.7±26.3% average C•G-to-G•C purity was observed across all tested CGBEs for cytosines positioned near the center of the editing window, with substantial variation across target sequences: the top 5% had >79.6% C•G-to-G•C purity while the bottom 5% had <1.0%. To decipher the sequence determinants that underly CGBE activity, simple motifs were computed for editing efficiency and transversion purity using a logistic regression model that considers each nucleotide independently (see FIG. 5G, Methods)¹². These motifs revealed that TC is strongly favored while GC is disfavored for editing efficiency across the tested CGBEs. Gradient-boosted regression trees were further trained to predict CGBE editing efficiency sequence context, which achieved good accuracy with R=0.57-0.77 at held-out target sites. Consistent with a previous characterization of BE4 variants¹², sequence motifs that associated RCTA with higher C•G-to-G•C purity (R=A or G) across all characterized CGBEs were observed. Cytosines in an ACTA motif were edited with an average C•G-to-G•C purity of 68.7% (N=1,760) across CGBEs, substantially higher than the 24.7% average across all sequence contexts, indicating a major role for sequence context in determining C•G-to-G•C editing outcomes. These simple target sequence motifs predicted 27.0%-53.3% of the variation in C•G-to-G•C purity.

Next, BE-Hive models were trained for these ten CGBEs (termed CGBE-Hive) and the models' ability to predict C•G-to-G•C editing purity at held-out sequence contexts not seen during training were evaluated. These models explained 58.3%-76.3% of the variance in C•G-to-G•C purity in the held-out dataset, a substantial improvement over logistic regression described above (27.0%-53.3%) (FIG. 60H). This performance improvement highlights that while C•G-to-G•C purity can be predicted using a simple motif such as RCTA that considers each nucleotide independently, higher-order interactions between nucleotides learned by deep neural networks substantially improve C•G-to-G•C editing purity predictions. Collectively, these observations establish that CGBE editing efficiency and purity can be accurately predicted by machine learning models.

To further investigate sequence determinants of CGBE editing outcomes, target sequence motifs for cytosines with the highest C•G-to-G•C efficiency for each CGBE were calculated (Methods). While most CGBEs shared sequence preferences favoring TC for overall editing efficiency and RCTA for purity, different CGBEs had distinct motifs that correlated with C•G-to-G•C yield. POLD2-APOBEC1-UdgX-nCas9-UdgX favored RCTA for C•G-to-G•C yield, while eA3A-nCas9 simply favored TC (FIG. 60I). Interestingly, RBMX-eA3A-UdgX-nCas9 favored CTC, while UdgX-EE-UdgX-nCas9-UdgX favored TCT, and Anc689-nCas9 favored CTA (FIG. 60I). These observations reveal that different CGBEs show distinct sequence preferences that influence the yield of C•G-to-G•C outcomes.

Machine learning models trained on up to 10,638 sgRNA-target pairs for these ten CGBEs are provided in an online interactive web app (crisprbehive.design)¹². Users can query sgRNAs and target sequences for data-driven predictions on editing outcomes of all CGBEs characterized herein.

### Model-Guided Correction of Pathogenic Transversion SNVs

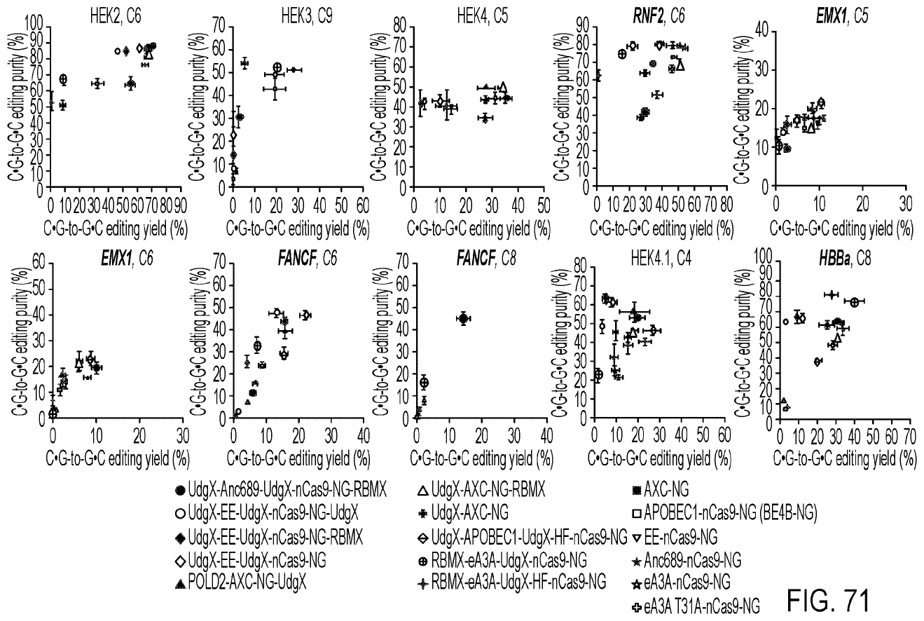

To extend the applicability of these CGBEs, their compatibility with PAM-variant Cas9 proteins were assessed. Editing at eight loci by CGBEs was evaluated using Cas9-NG, an engineered SpCas9 variant with broadened PAM compatibility³¹, and similar editing purities to SpCas9 CGBEs were observed at NGG PAM substrates (FIGS. 71, 72). The best performing NG-CGBEs at each locus retained >50% yield relative to SpCas9 CGBEs at targets with NGG PAMs (FIG. 71).

Given the broadened targeting scope of NG-CGBEs their performance was characterized on the “transversion-enriched SNV library”12 in mESCs, which contains 3,400 sgRNA-target pairs selected by BE-Hive from 18,523 disease-related G•C-to-C•G and A•T-to-C•G SNVs from the ClinVar and HGMD databases that are targetable by Cas9-NG^{1,32}, predicted to be correctable by cytosine transversion base editing with high purity and yield.

The following NG-CGBEs were generated based on their performance on the comprehensive context library: Anc689-nCas9-NG, APOBEC1-nCas9-NG, eA3A-nCas9-NG, UdgX-Anc689-UdgX-nCas9-NG-RBMX, and UdgX-APOBEC1-UdgX-HF-nCas9-NG. As Cas9-NG generally demonstrates reduced editing activity compared to wild-type SpCas931, similar to HF-Cas9, UdgX-APOBEC1-UdgX-nCas9-NG was included without the HF modifications as an alternative binding-impaired Cas9-fusion variant.

All six CGBEs tested on the transversion-enriched SNV library enabled high-purity C•G-to-G•C editing at disease-associated SNVs. At 247 cytosines predicted by CGBE-Hive to have >80% C•G-to-G•C editing purity, CGBEs demonstrated an average of 83% C•G-to-G•C editing purity (FIG. 61A). Each CGBE corrected >200 SNVs to their wild-type coding sequence with >90% precision among edited amino acid sequences (amino acid correction precision; FIG. 61B), with a total of 546 unique SNVs across CGBEs. For example, in the genome-integrated library, eA3A-nCas9-NG corrected the G•C-to-C•G SNV in COL3A1 associated with Ehlers-Danlos syndrome³³ with 71.4% yield and 92.8% purity, and corrected an SNV in BRCA2 associated with familial breast and ovarian cancer 34 with 66.5% yield and 82.5% purity. The fusion CGBE UdgX-APOBEC1-UdgX-nCas9-NG corrected an SNV in NSD1 associated with Sotos syndrome³⁵ with 40.0% yield and 73.4% purity and corrected an SNV in NIPBL associated with Cornelia de Lange syndrome³⁶ with 38.8% yield and 76.9% purity. Collectively, these results reveal efficient and high-purity correction of hundreds of disease-related SNVs by CGBEs.

Notably, the UdgX-APOBEC1-UdgX-nCas9 CGBE maintained a similar high purity of C•G-to-G•C editing between HF-nCas9 and nCas9-NG variants. UdgX-APOBEC1-UdgX-nCas9-NG, however, offered substantially better yield of genotype and coding sequence corrected G•C-to-C•G SNVs (FIGS. 61A-61B). These results suggest that fusion of CGBEs to Cas9-NG variants may obviate the need to use HF-variant Cas9-proteins to alter their binding kinetics to promote C•G-to-G•C editing outcomes.

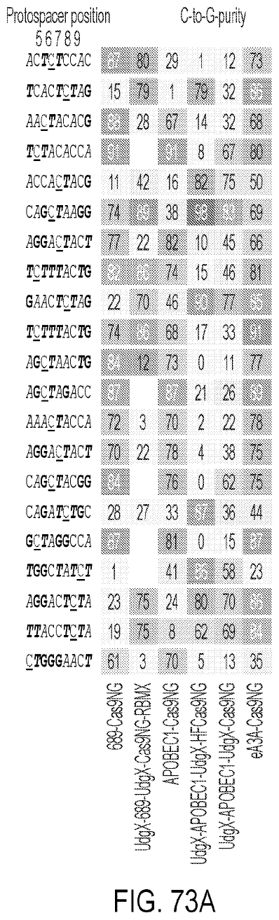

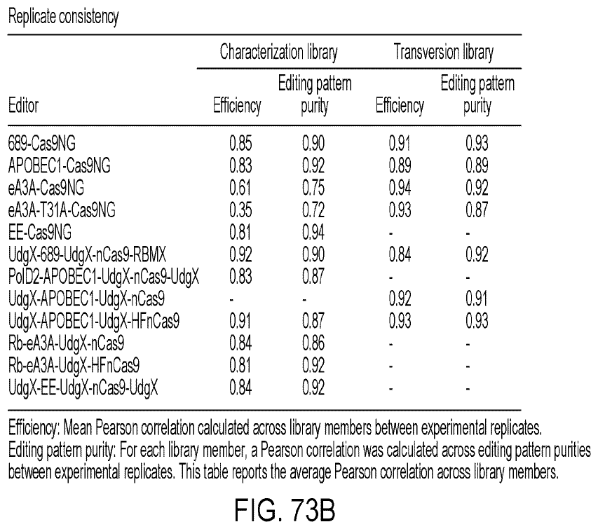

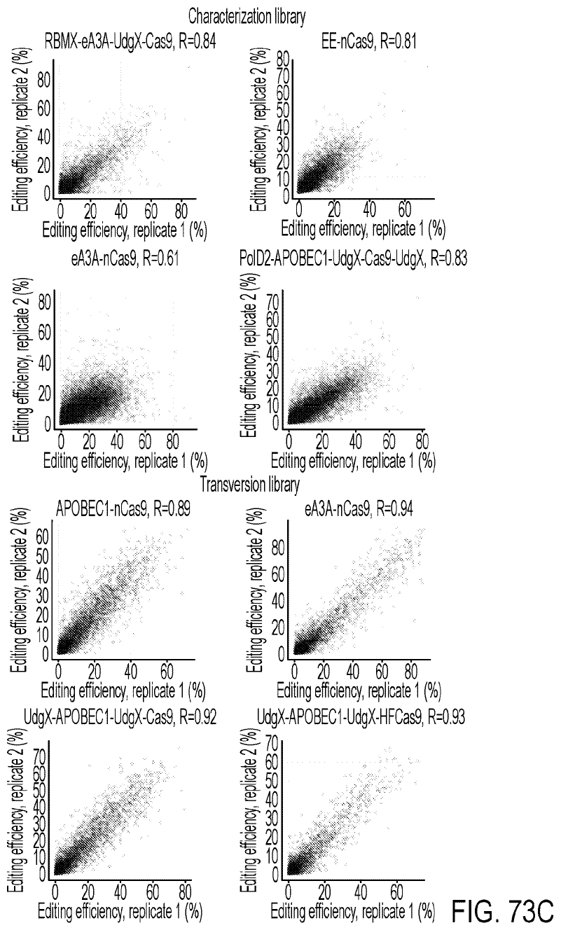

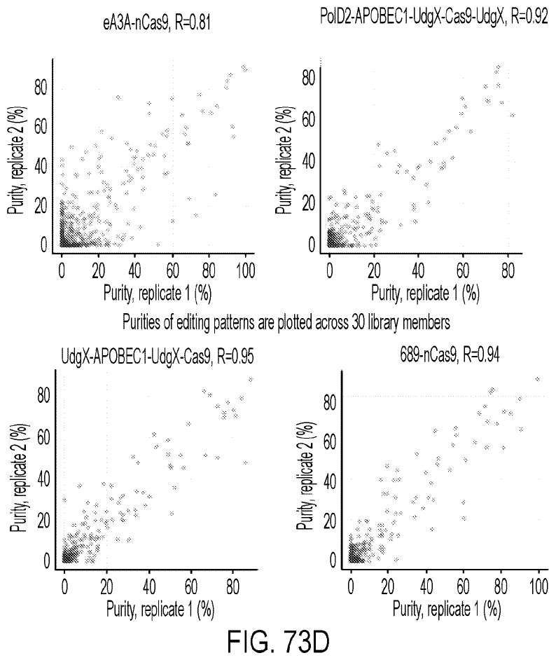

The best-edited targets in the transversion-enriched SNV library varied greatly by CGBE. Some SNVs edited with >90% purity by one CGBEs had purity below 5% for other CGBEs (FIGS. 73A-73B). CGBE-Hive models accurately accounted for this diversity in editing purity in the transversion-enriched SNV library, and accurately predicted the yield of exact genotype correction products and of alleles with corrected amino acid sequences (R=0.89-0.93 and R=0.91-0.94, respectively, FIG. 61C), as well as the DNA and amino acid correction precision (R=0.77-0.85 and R=0.82-0.90, respectively, FIG. 61D), including targets with multiple cytosines in the editing window. Since accurately predicting correction yield and precision requires accurate predictions for CGBE efficiency, C•G-to-G•C purity, and bystander editing patterns, these results establish that CGBE-Hive has learned important aspects of CGBE editing activity and can guide the use of CGBEs for high-purity correction of disease-related transversion SNVs.

Using CGBE-Hive to pick the best among the characterized CGBEs to correct each SNV should achieve greater C•G-to-G•C correction than applying any single CGBE to a set of targets. Indeed, it was observed that using CGBE-Hive to choose the three CGBE variants predicted to best achieve the desired edit (top-3 performance) increased the number of targets corrected with >90% precision or to >40% efficiency by 4.1- and 5.0-fold, respectively, compared to the number of targets that were expected to be corrected with these precision and efficiency thresholds by picking any single CGBE (FIG. 61E). These improvements of 4.1-and 5.0-fold by using the top three CGBE-Hive choices were nearly identical to the performance from picking the best CGBE out of all six options in hindsight. CGBE-Hive also displayed strong top-1 performance: Using CGBE-Hive to choose just a single CGBE increased the number of targets corrected with >90% precision or to >40% efficiency to 1.7-and 4.0-fold, respectively, compared to picking a single CGBE in expectation.

For correction precision, CGBE-Hive recovered the best performing CGBE variant in its top choice in 43.3% of targets and in its top three choices in 84.2% of target sequences.

For correction yield, CGBE-Hive recovered the best-performing CGBE variant in its top choice in 67.5% of targets and in its top three choices in 97.2% of targets. These results collectively demonstrate that this panel of CGBEs have diverse editing activities that CGBE-Hive has learned to predict, to optimize selection of the most promising CGBE variant to use for a desired edit. These improvements were also observed at endogenous loci in HEK293T cells (FIG. 61F).

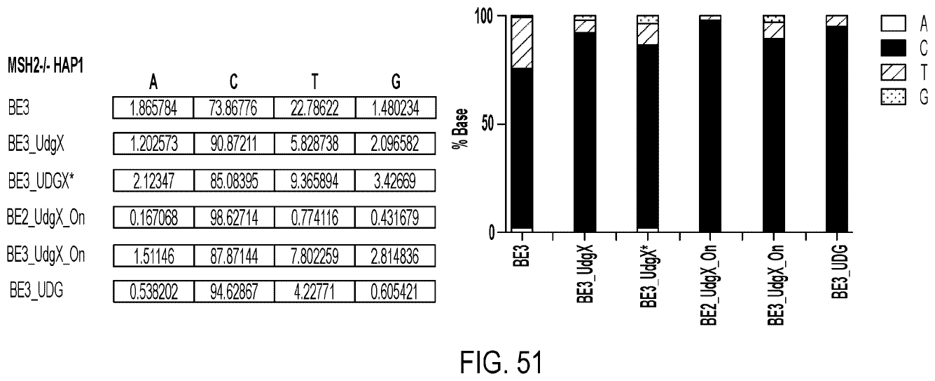

CGBE-Hive was used to identify disease-relevant C•G-to-G•C SNVs that could be installed in HEK293T cells using CGBEs characterized in this study. The CTNNB1 c.2138-1 G>C mutation, a cancer-associated allele, was installed by UdgX-APOBEC1-UdgX-HF with higher yield (64±1.0% vs. 51±0.5%) and purity (75±0.8% vs. 67±1.5%) than the best-performing simple deaminase-nCas9 fusion, Anc689-nCas9 (FIG. 61F). Additionally, the DIS3L2 c.2011-1 G>C mutation, associated with Perlmen Syndrome, was installed with higher purity by UdgX-Anc689-UdgX-nCas9-RBMX (46±1.1% vs. 41±1.3%) and similar editing efficiency (32±2.4% vs. 31±2.3%) compared to the best-performing deaminase-nCas9, eA3A-nCas9 (FIG. 51F). NG-CGBEs were also used to install a pathogenic SNV in the KCNQ2 gene predicted to be editable by CGBE-Hive with RBMX-eA3A-UdgX-nCas9, and observed 37.5±3.3% yield and 79.5±1.0% purity (FIG. 6F). These results indicate that CGBEs using both wild-type nCas9 and a Cas9 variant engineered to be compatible with non-native PAM sequences can efficiently install disease-associated alleles in human cells as predicted by CGBE-Hive. These results collectively demonstrate that the CGBEs developed in this study can install disease relevant SNPs with high efficiency and purity.

Thus, CGBE-Hive enables researchers to reap the benefits of the diversity of CGBEs developed in this study without the need to test all CGBE variants.

Comparisons with Recently Reported CGBEs, Prime Editing, and Off-Target Profiling

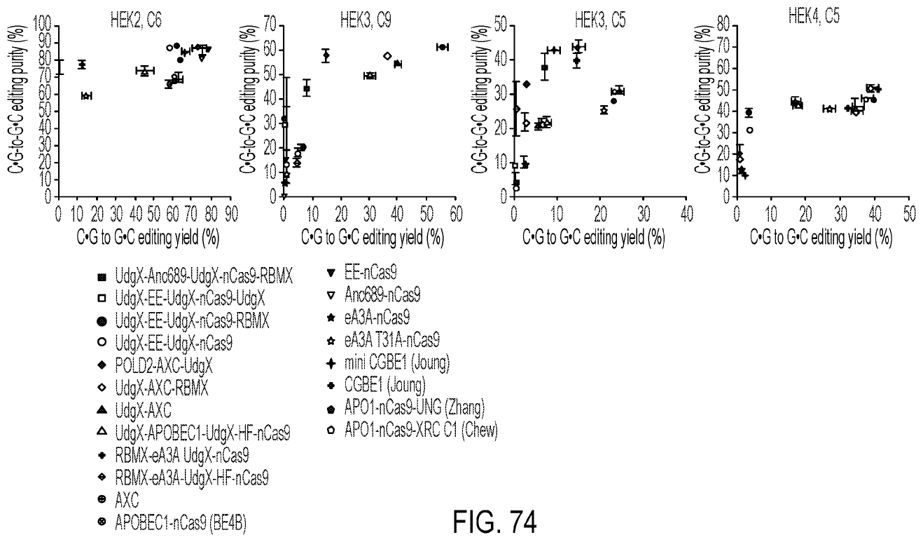

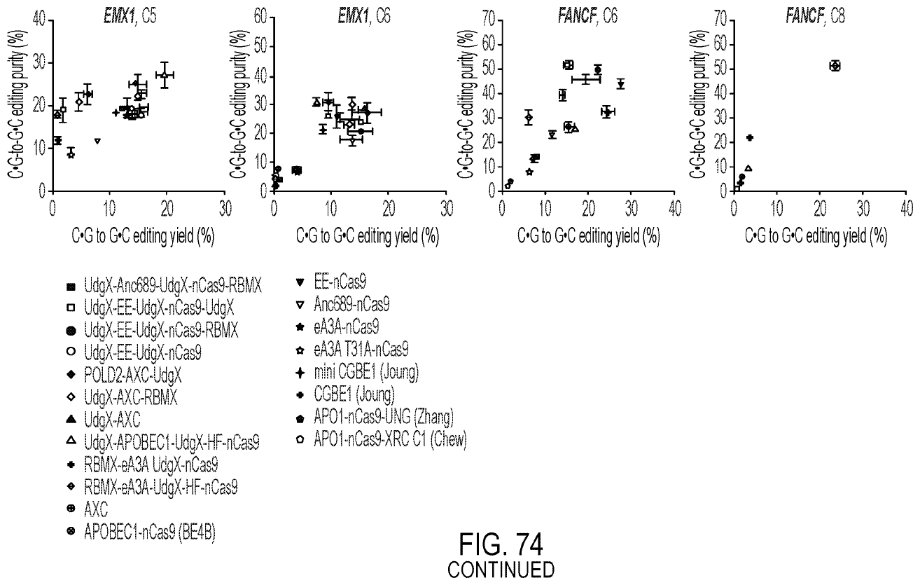

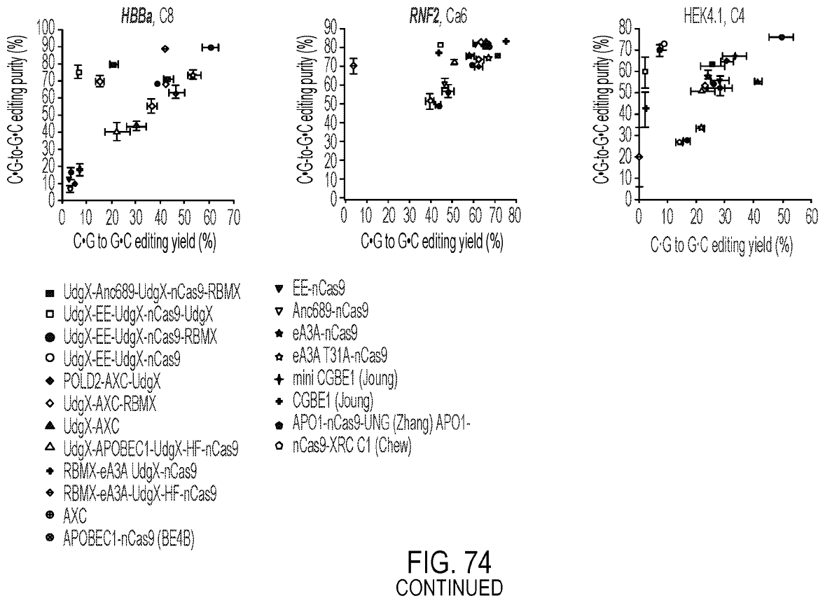

Next, it was determined whether the CGBE variants described in this work extend the scope of C•G-to-G•C base editing beyond those accessible with recently described CGBEs or prime editing (PE). It was found that the CGBEs developed in this study extend the scope of C•G-to-G•C genome editing by enabling higher yields and product purities at a wider array of target sequences compared to the use of previously described CGBEs alone except at loci already edited with high yield and purity by deaminase-nCas9 constructs (FIG. 74).

The editing activity of CGBEs developed herein were compared to previously described CGBEs2-4 (mini CGBE1, CGBE1, APOBEC1-nCas9-UNG, and APOBEC1-nCas9-XRCC1) across eight genomic loci in HEK293T cells. The CGBEs developed herein outperform previously described CGBEs at six of eight tested loci, with the broader sequence substrate scope of the CGBEs described in this work enabling efficient editing at a broader array of loci. For example, at HEK site 3 C9, UdgX-APOBEC1-UdgX-HF edits with 55.4±1.1% yield and 61.5±0.9% purity while the best previous CGBE (APOBEC1-nCas9-XRCC1) edits with 5.22±0.3% yield and 18.7±1.4% purity (FIG. 74). Additionally, at HBBa C8, RBMX-eA3A-UdgX-C edits with 60.6±3.0% yield and 88.9±1.4% purity while the best performing previous CGBE (CGBE1; eUNG-APOBEC1 R33A-nCas9) edits with 7.2±0.8% yield and 17.6±3.7% purity (FIG. 74). At the two sites, RNF2 and HEK4.1 that were very well edited by deaminase-nCas9 constructs, the CGBEs in this study performed comparably or modestly worse than the best previously reported CGBE. For RNF2, editing purity was comparable for CGBE1 and POLD2-APOBEC1-UdgX-nCas9-UdgX (CGBE1=82.8±0.9% vs. 82.1±1.4%) while yield improved to 74.8±0.4% for CGBE1 vs. 66.1±1.6% (FIG. 74). At HEK4.1, editing yield and purity for CGBE1 were 49.6±4.5% and 75.7±1.2%, respectively, compared with 41.7±1.0% and 55.0±1.2% for UdgX-APOBEC1-UdgX-nCas9 (FIG. 74).

Furthermore, it was observed that these novel CGBEs complement prime editing technology³⁷. Recently described prime editors (PEs) consist of Cas9 nickase fused to an engineered reverse transcriptase^{15,16}. See also International Publication No. WO 2020/191239, published Sep. 24, 2020, which is incorporated by reference herein. PEs are targeted to a genomic locus by an engineered prime editing guide RNA (pegRNA) that encodes both the desired edit and the target site.

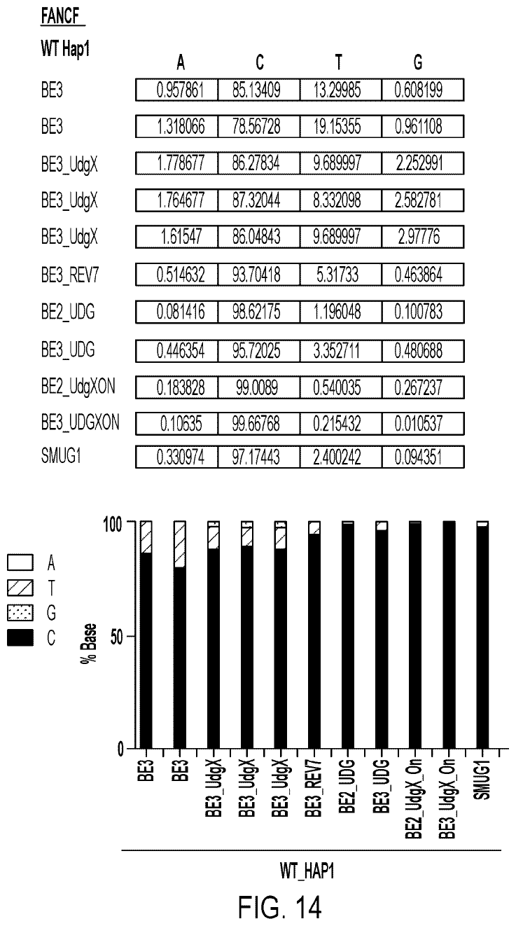

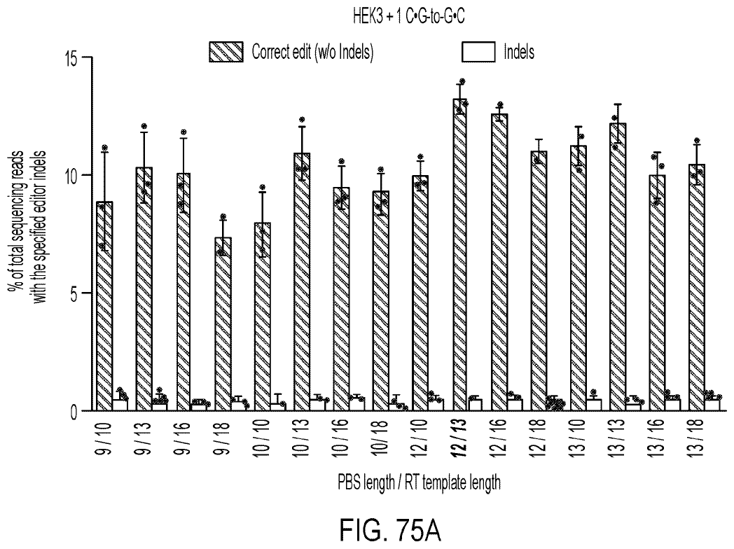

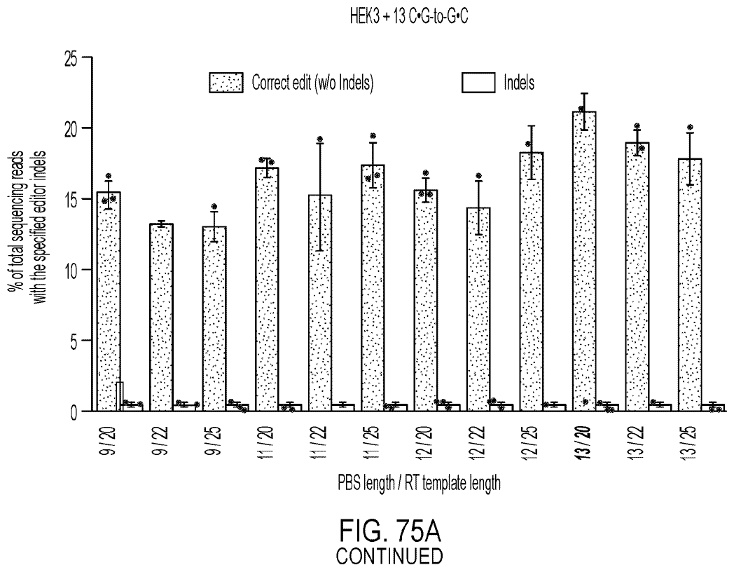

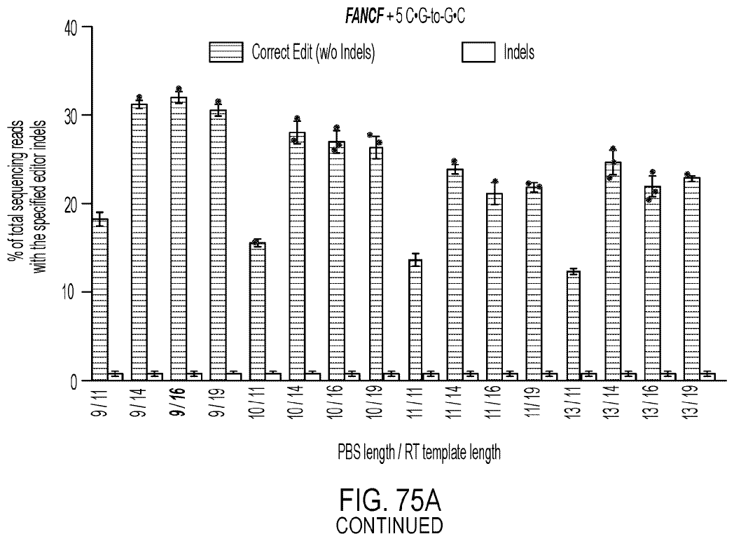

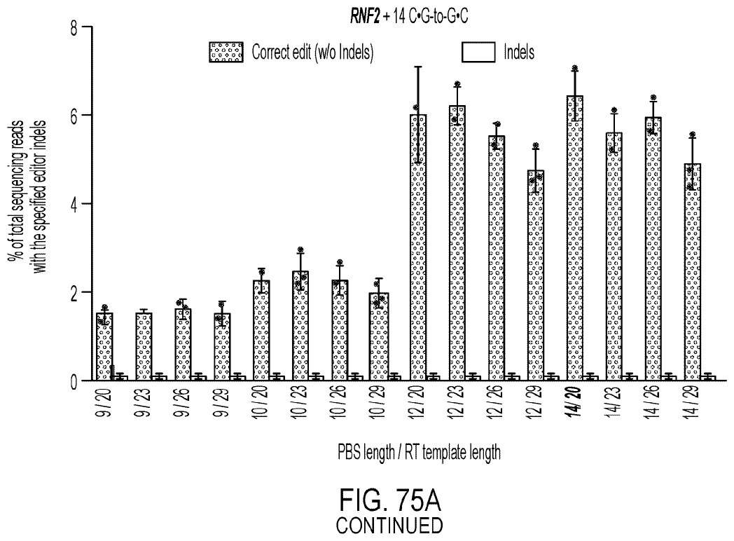

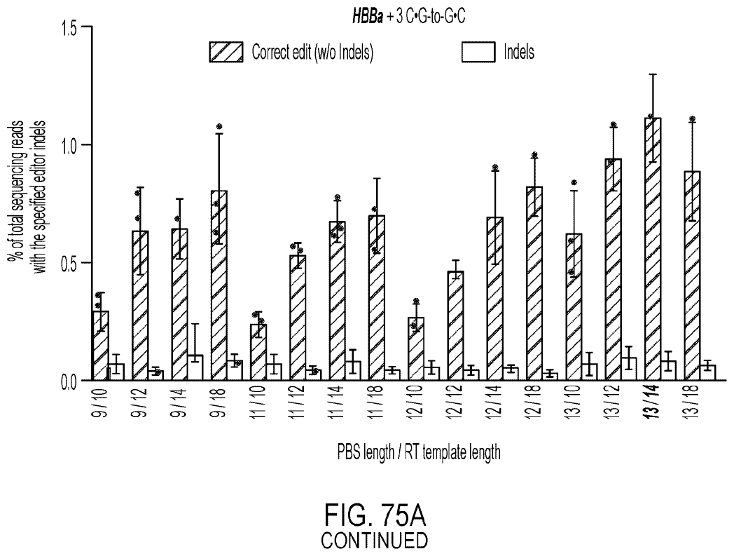

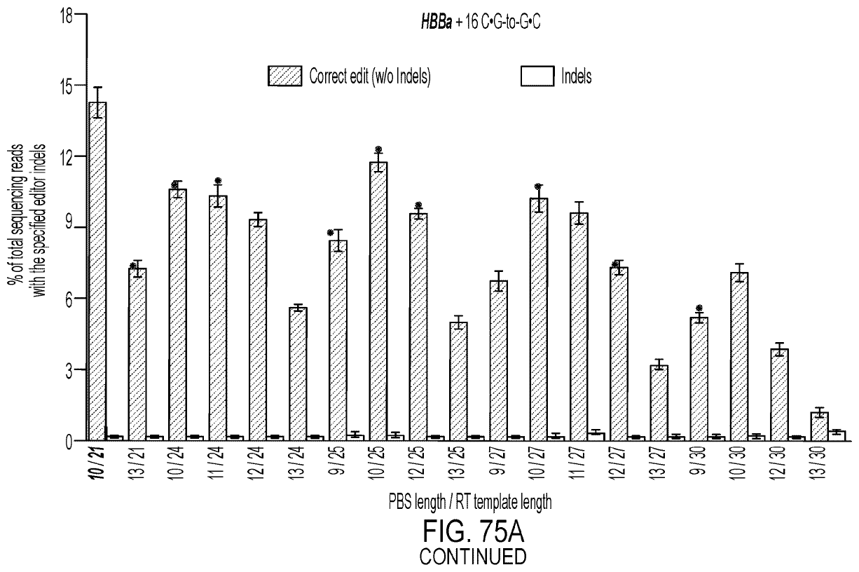

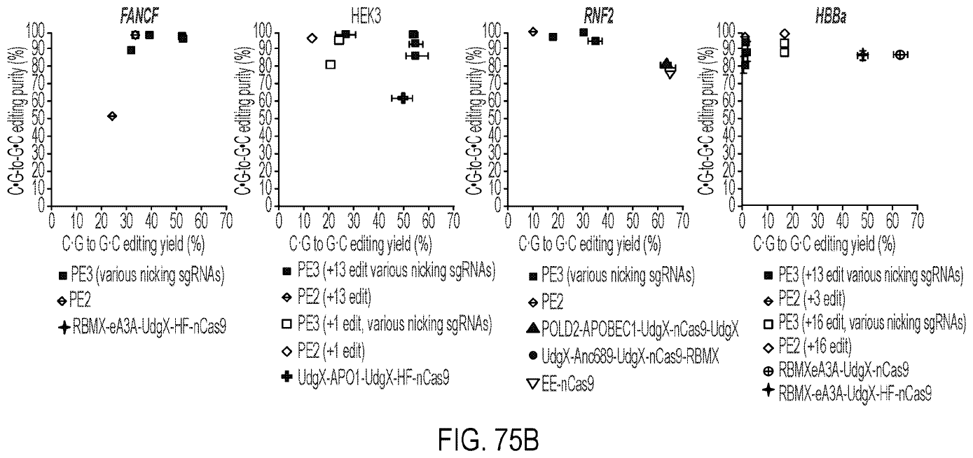

Since prime editing enables a broad range of genome edits including all 12 possible single-base conversions, as well as small insertions and deletions^{15,16}, it was sought to characterize how CGBEs and prime editors compare. Successful prime editing requires thorough optimization of the primer binding site (PBS) and the reverse transcriptase template in the pegRNA^{15,16}. These parameters were optimized for C•G-v to-G•C edits at four genomic loci (FANCF, HEK site 3, RNF2, and HBBa) (FIG. 14A). Each of these optimized pegRNAs were then tested using PE2, which does not nick the non-edited strand, as well as prime editor 3 (PE3), which nicks the non-edited strand by adding an additional sgRNA. The best-performing CGBE were also evaluated for these loci and editing efficiencies and product purities of CGBEs and PEs were compared at these loci. Two of the four loci (HEK site 3 and FANCF) were edited with higher efficiency and purity using PE compared with CGBEs. The best PE-mediated editing of the FANCF locus was 52.3±0.8% yield with 97.3±0.7% purity with PE3, while the best CGBE-mediated editing (with RBMX-eA3A-UdgX-HF) provided 24.4±0.6% yield and 52.7±2.8% purity. Likewise, the best balance of editing yield and purity by PE at the HEK site 3 locus was 54.3±1.8% yield with 98.2±0.1% purity with PE3, while the best CGBE editing (UdgX-APOBEC1-UdgX-HF) was 49.7±4.3% yield and 62.1±0.7% purity. At the other two loci (RNF2 and HBBa), however, the best-performing CGBEs characterized in this work provide the desired edits with higher efficiency than PE (FIG. 75B). At the RNF2 locus, PE3 installed the target nucleotide with 34.5±2.5% yield and 94.8±1.0% purity while CGBE (POLD2-APOBEC1-UdgX-C-UdgX) installed the same mutation with 62.5±2.3% yield and 81.7±1.7% purity. HBBa editing by PE proceeded with 17.2±1.1% yield and 98.9±0.63% purity with prime editor 2 (PE2) (slightly outperforming PE3) while CGBE (RBMX-eA3A-UdgX-C) edited with 64.0±2.1% yield and 88.3±1.6% purity (FIG. 75B). It was found that PE typically offers higher product purities while editing with CGBEs offers higher editing yields at some loci (FIGS. 75A-75B), consistent with recent reports^{13-15,37}. Notably, prime editing currently requires extensive optimization of pegRNA features to achieve high-efficiency edits, while CGBE-Hive prediction obviates CGBE editor selection. CGBEs complement prime editing for efficient C•G-to-G•C editing, although additional optimization of both technologies may further improve their properties.

Potential off-target editing outcomes of CGBEs were also characterized. Since the genome-wide off-targets of base editors that use cytosine deaminase enzymes are known to be predominantly sgRNA dependent, Cas9-dependent off-target editing profiles of CGBEs were characterized by examining the activity of CGBEs at previously confirmed off-target loci of corresponding Cas9:sgRNA complexes⁸. The architectural changes and protein fusions used to develop the CGBEs in this study resulted in lower Cas9-dependent off-target editing compared to corresponding CGBEs lacking protein fusions (FIG. 72, FIGS. 76A-76B), despite their generally higher on-target editing, perhaps because the more complex fusions or architectural changes introduce additional conformational requirements in editor:DNA complexes that are not met by some off-target loci. CGBE off-target editing activity was examined at thirteen off-target loci for four sgRNAs (HEK site 2, HEK site 3, HEK site 4, and FANCF). On-target editing efficiency was confirmed and is shown in FIG. 72. While off-target editing varied by site, as has been reported previously¹⁷, the deaminase domain was the primary determinant of off-target editing activity. Across all cytidines assessed within a broadened search window (protospacer positions C1-C12) to capture all possible off-target edits, an average off-target nucleotide modification frequency of 5.9±0.5% for eA3A-nCas9, 6.4±0.3% for EE-nCas9, 11.9±0.9% for APOBEC1-nCas9, and 13.0±0.3% for Anc689-nCas9 was observed (FIGS. 76A-76B). Importantly, the average frequency of off-target in-window editing (any C•G to T•A, A•T, G•C, or indel at an in-window off-target cytosine) across the thirteen studied off-target loci was substantially decreased for our engineered CGBE variants tested compared to the corresponding simple deaminase-nCas9 fusions (FIGS. 76A-76B). For example, RBMX-eA3A-X-C showed a 4.5-fold reduction in off-target editing compared to eA3A-nCas9, while the RBMX-eA3A-X-HF construct, which has a slightly shifted editing window, showed a large 52-fold reduction relative to eA3A-nCas9. Among the 16 characterized CGBE variants containing protein fusions made in this study, off-target editing levels on average were 11.3-fold lower than the corresponding deaminase-nCas9.

Together, these results indicate that the novel protein fusion CGBEs developed herein offer lower Cas9-dependent off-target editing compared to corresponding CGBEs lacking those fusions, despite their generally higher on-target editing, perhaps because the more complex fusions introduce additional conformational requirements in editor:DNA complexes that are not met by some off-target loci.

Base editor off-target activity may also arise in a sgRNA-independent manner. Such edits are predominantly driven by the deaminase component; therefore, it is anticipated that sgRNA-independent off-target activity of CGBE will mirror that of the CBEs that use the same cytosine deaminase. While overexpression of fusion proteins, including DNA repair proteins, as CGBE-components may result in additional sgRNA-independent off-target effects, these are likely to differ, perhaps due to cell-type specific DNA repair profiles, and are therefore best assessed per application.

While DNA repair protein CGBE components may result in additional Cas-independent off-target effects, these are likely to differ by cell type and delivery method, and therefore are best assessed for each application.

### Discussion

Understanding and controlling the outcomes of genome editing experiments are important challenges for achieving targeted, precise genome manipulation. Molecular determinants of transversion base editing was investingated, including the effects of the deaminase and Cas effector domains, as well as many DNA repair proteins, and these insights were used to engineer novel CGBEs. The editing outcomes and performance of these reagents were characterized using a high-throughput genome-integrated library assay in mammalian cells and sequence features that affect base editing outcomes of ten diverse CGBEs were identified. It was shown that C-to-G editing activity was predicted with substantially higher accuracy by deep learning models compared to simpler models, indicating that complex sequence features drive C•G-to-G•C editing activity.

Provided herein are trained CGBE-Hive machine learning models which accurately predict CGBE efficiency, C•G-to-G•C editing purity, and bystander editing patterns (R=0.90) to enable predictable and consistently pure CGBE editing. A machine learning workflow was demonstrated using CGBE-Hive to identify optimal CGBE and sgRNA editing strategies to install a desired edit and show that this workflow expands high-efficiency and high-purity C•G-to-G•C editing to more loci than using any single CGBE by 5.0-fold and 4.1-fold with the top three CGBE-nominated choices. CGBE-mediated correction of the amino acid sequences of 546 disease-associated single nucleotide variants (SNVs) was demonstrated with >90% precision. Furthermore, efficient and pure installation of four disease-relevant SNPs was demonstrated and the performance of these tools was tested in other mammalian cell lines. Collectively, the base editor and computational tools presented herein substantially improve the targeting scope, effectiveness, and utility of CGBE-mediated transversion base editing.

### Data and Code Availability

The target library sequencing data generated during this study are available at the NCBI Sequence Read Archive database under PRJNA631290. Data from the Repair-seq screens are available under PRJNA721212. Processed target library data used for training machine learning models have been deposited under the following DOIs: 10.6084/m9.figshare.12275645 and 10.6084/m9.figshare.12275654.

### Code Availability

Code used for analyzing CRISPRi screens is available at github.com/jeffhussmann/repair-seq. Code used for target library data processing and analysis are available at github.com/maxwshen/lib-dataprocessing and github.com/maxwshen/lib-analysis. The machine learning models for CGBEs trained on target library data are available as a part of the BE-Hive interactive web application at crisprbehive.design and the BE-Hive Python package at github.com/maxwshen/be_predict_efficiency.

### Methods

### General Methods

DNA oligonucleotides were obtained from Integrated DNA Technologies (except where otherwise specified). All mammalian editor plasmids used in this work were cloned by Gibson assembly according to manufacturer's protocols. Except for the CRISPRi library, plasmids expressing sgRNAs were constructed by ligation of annealed oligonucleotides into BsmBI-digested acceptor vector as previously described^{18,19}. Plasmids expressing pegRNAs were constructed by Golden Gate assembly using a custom acceptor plasmid as previously described¹⁵. Protospacer sequences of sgRNAs used for non-library experiments in this work are listed in Table 6. pegRNA protospacer and extension sequences are listed in Table 5. Vectors for low-throughput mammalian cell experiments were purified using Plasmid Plus Midiprep kits (Qiagen) or PureYield plasmid miniprep kits (Promega), which include endotoxin removal steps. Cloning of the CBE SaCas9 sgRNA for screening was conducted by KLD assembly according to the manufacturer's protocol using BPK2660 (Addgene #70709) as a template with the following primers: GGTGTTTCGTCCTTTCCACAAGATA (SEQ ID NO: 224), gCTGATAGGCAGCCTGCACTGGGTTTTAGTACTCTGTAATGAAAATTACAGAATC TAC (SEQ ID NO: 225).

### General Mammalian Cell Culture Conditions

HEK293T (ATCC CRL-3216), U20S (ATTC HTB-96), K562 (CCL-243), and HeLa(CCL-2) cells were cultured and passaged in Dulbecco's Modified Eagle's Medium (DMEM) plus GlutaMAX (ThermoFisher Scientific), DMEM (Gibco), McCoy's 5A Medium (Gibco), RPMI Medium 1640 plus GlutaMAX (Gibco), or Eagle's Minimal Essential Medium (EMEM, ATCC), respectively, each supplemented with ˜10% (v/v) fetal bovine serum (Gibco, qualified) and 1× Penicillin Streptomycin (Corning). All cell types were incubated, maintained, and cultured at 37° C. with 5% C02. Cell lines were authenticated by their respective suppliers or short tandem repeat profiling and tested negative for mycoplasma. Culturing conditions for library analyses are detailed below. Lentivirus was produced in HEK293T cells by co-transfection with packaging plasmids encoding gag and pol, rev, and tat from HIV-1 and VSVG envelope protein. For these transfections, either TranslT®-LT1 Transfection Reagent (Mirus) or Polyethylenimine (PEI; Polysciences, Inc.) were used.

### HEK293T Tissue Culture Transfection (Non-Viral) Protocol and Genomic DNA Preparation

HEK293T were cells grown, seeded, and transfected as previously described^{5,6,15,18-20}. Briefly, cells were trypsinized and seeded on 48-well poly-D-lysine coated plates (Corning) to an approximated of 3×105 cells per well. 16-24 h post-seeding, cells were transfected at approximately 60% confluency with 1 μL of Lipofectamine 2000 (Thermo Fisher Scientific) according to the manufacturer's protocols and 750 ng of base editor plasmid and 250 ng of sgRNA plasmid. For Prime editing experiments, non-nicking conditions were carried out with 750 ng of PE2 and 250 ng pegRNA while nicking experiments included an additional 83 ng of nicking sgRNA. 72 h post-transfection, media was removed, cells were washed with 1×PBS solution (Thermo Fisher Scientific), and genomic DNA was extracted by the addition of 150 μL of freshly prepared lysis buffer (10 mM Tris-HCl, pH 7.5; 0.05% SDS; 25 μg/mL Proteinase K (ThermoFisher Scientific)) directly into each well of the tissue culture plate. The genomic DNA•lysis buffer mixture was incubated at 37° C. for 1 h, followed by an 80° C. enzyme inactivation step for 30 min. Primers used for mammalian cell genomic DNA amplification are listed in Table 6. Protospacer sequences used for each locus are listed in Table 6.

### High-Throughput DNA Sequencing of Genomic DNA Samples

Genomic sites of interest were amplified from genomic DNA prepared and sequenced on an Illumina MiSeq as previously described^{5,6,15,18-20} with minor modifications. Briefly, amplification primers containing Illumina forward and reverse adapters (Table 6) were used for PCR 1, amplifying the genomic region of interest. PCR 1 reactions were performed with 0.5 μM of each forward and reverse primer, 1 μL of genomic DNA extract, 3% DMSO, 0.25 μL Phusion HS-II polymerase, 5 μL Phusion HF buffer, 0.5 μL 10 mM dNTPs, and water to a final volume of 25 μL. PCR1 reactions were carried out as follows: 98° C. for 2 min, then 32 cycles of [98° C. for 10 s, 61° C. for 20 s, and 72° C. for 30 s], followed by a final 72° C. extension for 2 min. Unique Illumina barcoding primer pairs were added to each sample in a secondary PCR reaction (PCR 2). Specifically, 25 μL of a given PCR 2 reaction contained 0.5 μM of each unique forward and reverse Illumina barcoding primer pair, 1 μL of unpurified PCR 1 reaction mixture, 0.25 μL Phusion HS-II polymerase, 5 μL Phusion HF buffer, 0.5 μL 10 mM dNTPs, and water to a final volume of 25 μL. The barcoding PCR 2 reactions were carried out as follows: 98° C. for 2 min, then 12 cycles of [98° C. for 10 s, 61° C. for 20 s, and 72° C. for 30 s], followed by a final 72° C. extension for 2 min. PCR products were evaluated by electrophoresis on 2% agarose gel. PCR 2 products (pooled by common amplicons) were purified by electrophoresis with a 2% agarose gel using a QIAquick Gel Extraction Kit (Qiagen), eluting with 40 μL of water. DNA concentration and library preparation was performed as previously described¹⁵ by fluorometric quantification (Qubit, ThermoFisher Scientific) and diluted to 4 nM final library concentration before sequencing on an Illumina MiSeq instrument according to the manufacturer's protocols.

Sequencing reads were demultiplexed using MiSeq Reporter (Illumina). Alignment of amplicon sequences to a reference sequence was performed using CRISPResso221 which was run to calculate indels with a window size of 10. C•G-to-G•C editing purity was calculated as C•G-to-G•C editing yield÷[C•G-to-T•A yield+C•G-to-A•T yield+indels].

### Nucleofection of HAP1, U2OS, K562, and HeLa Cells

Nucleofection was performed on K562, HeLa, and U20S cells as previously described¹⁵. 750ng of base editor-expression plasmid and 250ng sgRNA-expression plasmid were nucleofected in a final volume of 20 uL in a 16-well nucleocuvette strip (Lonza). K562 cells were nucleofected using the SF Cell Line 4D-Nucleofector X Kit (Lonza) with

5×10⁵ cells per sample (program FF-120), according to the manufacturer's protocol. U20S cells were nucleofected using the SE Cell Line 4D-Nucleofector X Kit (Lonza) with 3-4×10⁵ cells per sample (program DN-100), according to the manufacturer's protocol. HeLa cells were nucleofected using the SE Cell Line 4D-Nucleofector X Kit (Lonza) with 2×10⁵ cells per sample (program CN-114), according to the manufacturer's protocol. Nucleofiection of HAP1 cells was performed using the same amounts of DNA and final volume in a 16-well nucelocuvette strip; however, HAP1 cells were nucleofected using the SE Cell Line 4D-Nucleofector X Kit (Lonza) with 4×105 cells per sample (program DZ-113), according to the manufacturer's protocol. Cells were harvested 72 hours after nucleofection for genomic DNA extraction.

### Selection of Ten CGBEs for Target Library Characterization

The most representative and diverse subset of CGBEs were selected from endogenous base editing data for 72 CGBEs at eight or 16 endogenous target loci. Briefly, a convex relaxation of a quadratic program was used to find a subset of CGBEs with maximally diverse transversion editing purities and yields. Clustering analysis was used to suggest the number of unique CGBE families. Analytic results were curated manually. The six fusion CGBEs assayed were: PolD2-APOBEC1-UDGX-Cas9-UDGX, RBMX-eA3A-UDGX-Cas9, RBMX-eA3A-UDGX-HF-nCas9, UDGX-Anc689-UDGX-Cas9-RBMX, UDGX-APOBEC1-UDGX-HF-nCas9, and UDGX-EE-UDGX-Cas9-UDGX. The four simple CGBE editors were deaminase-nCas9 with eA3A, Anc689, APOBEC1, and EE deaminases. eA3A-T31A-nCas9 and eA3A-BEN3-ΔN13-UGI were also assayed. eA3A-nCas9, eA3A-T31A-nCas9 and eA3A-BEN3-ΔN13-UGI were characterized in the comprehensive context library only in HEK293T, while all other CGBEs were characterized in the comprehensive context library only in mESCs. eA3A-nCas9-NG and eA3A-T31A-nCas9-NG were further characterized in the transversion-enriched SNV library in mESCs.

To identify CGBEs with distinct activities, quadratic programming was used to identify a subset of CGBEs with maximum pairwise distances between vectors of C•G-to-G•C editing purity and yield across eight or 16 endogenous loci. Hierarchical clustering was also performed, and it was observed that across these endogenous loci, CGBE editing activity primarily clustered by deaminase, though there were also substantial intra-cluster differences in editing activities due to variety in protein fusion architectures that were occasionally larger than inter-cluster differences, which indicates that CGBE editing activity is affected by both deaminase and protein fusion architectures. As the quadratic programming and clustering methods only consider numerical distances and do not propose subsets optimized for high purity or yield, the quadratic programming results were manually curated by replacing CGBEs with similar neighbors from hierarchical clustering when the neighbors had meaningfully higher purity or yield. Since deaminases, protein fusions, and high-fidelity Cas9 variants are known to alter base editing activity^{2-4,8,22}, the final subset was also manually curated to ensure a diversity of these elements.

### CRISPRi Library Construction

For the CRISPRi screen a platform called Repair-seq was used, which was developed by Hussmann et al. using a CRISPRi guide library (see Hussmann et al., Cell (2021) 184(22), 5653-5669.e25, which is incorporated by reference herein). This library contains 1513 gene-targeting sgRNAs selected from hCRISPRi-v2.1²³ and 60 non-targeting controls selected from hCRISPRi-v2²³. Gene-targeted sgRNAs were against 476 genes enriched for ones involved in DNA metabolic processes (e.g., replication, repair, recombination). A minority of the spacer sequences for the gene-targeting sgRNAs in this library were repeated in hCRISPRi-v2.1 and are therefore annotated as targeting multiple gene promoters, with multiple guide identifiers. The 476 gene count considers only the first set of annotations. Oligonucleotides containing sgRNA targeting sequences were synthesized by Twist Bioscience.

### CRISPRi Library Cloning

The guide library was cloned in pAX198 as previously described in Hussmann et al. (2021). This vector was derived from pU6-sgRNA EF1Alpha-puro-T2A-BFP24 (Addgene, 60955) through multi-step molecular cloning. pAX198 contains a CRISPRi guide expression cassette driven by a modified mouse U6 promoter and ending with a termination signal consisting of 6 Ts. pAX198 also contains a ‘target region’ for genome editing derived from sequence at the human HBB gene, specifically the second and third exons of HBB (no intron) and part of the 3′UTR (ENST00000647020.1). This region is where Anc689-nCas9 and Anc589-dCas9 were directed (see CRISPRi screen cell culture section of Methods). Prior to library cloning, a BstXI site was removed from the target region by site-directed mutagenesis. Library cloning was performed with standard protocols (details available at weissmanlab.ucsf.edu/CRISPR/Pooled_CRISPR_Library_Cloning.pdf). Briefly, library oligonucelotides were amplified by PCR (primers 5′-TATGAACCACTAAGGCGTCCAC (SEQ ID NO: 226), 5′-TCACCAGCAGACTTTACGCAGC (SEQ ID NO: 227)), purified using MinElute Reaction Cleanup Kit (Qiagen), digested with BlpI and BstXI, isolated by gel purification, and ligated into a similarly digested expression vector (insert to backbone ratio of 1:1 for 16 hours at 16° C.). Ligation reactions were electroporated into MegaX DH10B T1R Electrocomp™ cells (ThermoFisher). Cells were grown on agar plates and then scraped into liquid for plasmid purification. The final sgRNA library (AX227) was verified by sequencing.

### CRISPRi Screen Cell Culture

The Repair-seq screens reported here were performed in previously described HeLa cells25, which stably express a dCas9-BFP-KRAB fusion (from pHR-SFFV-dCas9-BFP-KRAB; Addgene #46911), in two rounds. The first round of screening evaluated Anc689-nCas9. The second round evaluated Anc689-dCas9. Both rounds of screening were conducted as follows: Cells were transduced with guide library (AX227, see CRISPRi library cloning section below) by lentiviral infection. The infections were carried out in DMEM supplemented with ˜10% (v/v) fetal bovine serum, lx Penicillin Streptomycin, and 8 μg/mL polybrene at an observed infection efficiency of ˜5% for both Anc689-nCas9 and Anc689-dCas9, as determined by flow cytometry. Approximately 2 days post transduction, cells were selected in 3 μg/mL puromycin and then, 3 days later, transfected with plasmids for base editing. Each screen was performed in replicates, each split one day prior to transfection onto 30 15 cm plates, each containing ˜1.2×10⁶ cells. The transfection procedure was as follows: (1) 25 ng plasmid DNA (75% editor plasmid; 25% sgRNA plasmid) was mixed with 3.5 mL of Opti-MEM (Gibco) and 4.6 mL Helafect Transfection Reagent (per 15 cm plate of cells). (2) This mixture was then incubated at room temperature for 20 minutes and (3) added to DMEM (Gibco) supplemented with ˜10% (v/v) fetal bovine serum (20 mL per plate). (4) The prepared media was then used to replace non-transfection media on each plate of cells. Approximately 3 days later, cells were collected for sample preparation. For all arms of screening, ˜100×10⁶ cells or more were collected at a viability of >85%.

### CRISPRi Screen Sample Preparation

Sequencing libraries were prepared from cells collected at the end of the CRISPRi screens as follows: Genomic DNA was extracted from cell pellets (-200×10⁶ cells for each replicate of Anc689-nCas9, and 125×10⁶ and 98×10⁶ cells for each of two replicates of Anc589-dCas9) using the NucleoSpin® Blood XL kit (Macherey-Nagel, up to 100×10⁶ cells per column). The genomic DNA was fragmented by digestion with NotI-HF (NEB) and then enriched for edit-containing fragments (1447 bp) by size selecting each sample on a large 0.8% agarose gel (Owl™ A1 Large Gel System, Thermo Fisher Scientific). Gel electrophoresis was conducted at large-scale (i.e., with wells large enough to hold 1.5 mL volume per well) to maximize recovery of fragments containing both edited sequences and sgRNA expression cassettes (‘target’ fragments). Gel preparation details are available at https://weissmanlab.ucsf.edu/CRISPR/IlluminaSequencingSamplePrep_old.pdf. DNA was then isolated from excised regions of the gel using NucleoSpin® Gel and PCR Clean-up kit (Macherey-Nagel) with columns placed on a vacuum manifold. Of note, large sample volumes were passed through individual columns using syringe barrels to increase capacity.

Next, size-selected target fragments were prepared for sequencing using custom adaptors compatible with next-generation sequencing technologies from Illumina. These adapters, which contained 12 nt unique molecular identifiers (UMIs), were made by annealing individual DNA oligonucleotides (obtained from Integrated DNA Technologies). The oligonucleotide components were oBA676 (5′-G*G*C*C*AGATCGGAAGAGCGTCGTGTAGGGAAAGAGTGTAGATCTCGGTGGTC GCCGTATCATT (SEQ ID NO: 228), HPLC purified) and oBA677 (5′-CAAGCAGAAGACGGCATACGAGATNNNNNNNNNNNNGTGACTGGAGTTCAGAC GTGTGCTCTTCCGATCT (SEQ ID NO: 229), HPLC purified), where * represents a phosphorothioated DNA base. Prior to ligation, DNA samples were digested with HindIII-HF (NEB). This step removed a 4 nt NotI overhang from one end of the target fragments, leaving only one side available for adaptor ligation. DNA was then purified using SPRIselect Reagent (Beckman Coulter) in a 0.8X reaction, quantified using Bioanalyzer High Sensitivity DNA Analysis (Agilent), and 1 μg of the product was ligated to adaptors using enzyme and buffer from the KAPA HyperPrep Kit (Roche) as follows: 30 μL ligation buffer, 10 μL ligase, adapter at 200:1 adaptor:insert ratio, and PCR-grade water to 110 μL total volume. These reactions were incubated at 4° C. overnight on a thermocycler with lid temperature set to 30° C.

Following ligation, DNA was purified using SPRIselect Reagent (Beckman Coulter) in two reactions (0.65× followed by 0.8×) and target fragments were enriched by PCR as follows: 30 ng of template, amplification primers at 0.6 μM final concentration (each), 3% dimethyl sulfoxide, and 1×KAPA HiFi HotStart ReadyMix (50 μL total volume) run at 1 cycle of 3 minutes at 95° C.; 16 cycles of 15 seconds at 98° C., followed by 15 seconds at 70° C.; 1 cycle of 1 minute at 72° C.; 4° C. hold. Enough PCR reactions were performed to use nearly the entirety of each sample obtained from the ligation and subsequent clean-up reactions. Amplification primers used were oBA679 (5′-CAAGCAGAAGACGGCATACGAGAT (SEQ ID NO: 230)) and 5′-AATGATACGGCGACCACCGAGATCTACAC-[index]-TCGTCGGCAGCGTCAGATGTGTATAAGAGACAGTATCCCTTGGAGAACCACCTTG TTGG (SEQ ID NO: 231). Amplified DNA was purified using SPRIselect Reagent (Beckman Coulter) in a 0.8× reaction, and index samples were mixed for sequencing. Throughout sample preparation procedures, samples were checked for quality and yield using either a NanoDrop Spectrophotometers (Thermo Fisher Scientific), Agilent 2100 Bioanalyzer system, or by running on a Novex™ TBE Gel. Sample preparation procedures are also described in Hussmann et al. (2021).

### CRISPRi Screen Analysis

Sequencing of CRISPRi screens, alignment and classification of screen sequencing data, statistical tests of gene significance in FIG. 57D and FIGS. 65A-65E, and identification of the top two most active guide RNAs for relevant genes in FIG. 57D and FIGS. 66A-66b were performed as described in Hussmann et al. (2021). Intervals in FIG. 64C are 95% Clopper-Pearson intervals of outcome fractions, converted to corresponding log 2 fold changes. That is, given k observed UMIs for a given CRISPRi guide in a numerator outcome set out of n total UMIs in a denominator outcome superset, the bottom interval (vbottom) is the smallest value of the true population proportion of numerator to denominator outcomes such that there is <=2.5% chance of observing >=k from Binomial(vbottom, n), and the top interval (vtop) is the largest value of the true population proportion of numerator to denominator outcomes such that there is <=2.5% chance of observing <=k from Binomial(vtop, n).

### Target Library Cloning

The target libraries used in this manuscript were previously generated in Arbab, Shen, et al., 2020¹², which is incorporated by reference herein. All editors described in this paper were cloned between the N-terminal and C-terminal NLS sequences flanking the eA3A-BE4max (Addgene 152997).

### Target Library Cell Culture

mESC lines used have been described previously and were cultured as described previously²⁶. For stable Tol2 transposon-mediated library integration, cells were transfected using Lipofectamine 3000 (Thermo Fisher) following standard protocols with equimolar amounts of Tol2 transposase plasmid and transposon-containing plasmid. For library applications, 15-cm plates with 2×107 initial cells were used. To generate library cell lines with stable Tol2-mediated genomic integration, cells were selected with 150 μg/mL hygromycin starting the day after transfection and continued for >2 weeks. For editing experiments, CGBEs were transfected with Tol2 transposase plasmid using Lipofectamine 3000 and selected with 10 μg/mL blasticidin starting the day after transfection for 4 days before harvesting. An average coverage of >300× per library cassette was maintained throughout.

### Target Library High-Throughput Sequencing

Library preparation was performed as described in Arbab, Shen et al. 20208. Genomic DNA was collected from cells 5 days after transfection, after 4 days of antibiotic selection. For library samples, 20 μg gDNA was used for each sample and an average sequencing depth of >4,000× per target was maintained. All PCRs were performed using NEBNext Ultra II Q5 Master Mix. Samples were pooled using Tape Station (Agilent) and quantified using a KAPA Library Quantification Kit (KAPA Biosystems). The pooled samples were sequenced using Illumina NextSeq.

### Target Library Analysis: Data Processing

Sequencing reads were assigned to designed library target sites by locality sensitive hashing^{8,27}. Target contexts that were intentionally designed to be highly similar to each other were designed barcodes to assist accurate assignment. Sequence alignment was performed using Smith-Waterman with the parameters: match +1, mismatch −1, indel start −5, indel extend 0. Nucleotides with PHRED score below 30 were assumed to be the reference nucleotide.

For base editing analysis, aligned reads with no indels were retained for analysis and events were defined as the combination of all possible substitutions at all substrate nucleotides in the target site in a read, where a single sequencing read corresponds to an observation of a single event. Substrate nucleotides were defined as C and G for CBEs and A and C for ABEs.

For indel analysis, reads containing indels with at least one indel position occurring between protospacer positions ˜6 to 26 were retained, where position 1 is the 5′-most nucleotide of the protospacer, and 0 is used to refer to the position between −1 and 1. Reads containing indels without at least six nucleotides with at least 90% match frequency on both sides of each indel were discarded. Events were defined as indels identified by position, length, and inserted nucleotides occurring in a read. Combination indels were either not observed at all or only at exceedingly low frequencies in endogenous data and were therefore excluded from consideration when analyzing library data.

### Target Library Analysis: Base Editing Profiles

Base editing profiles were calculated using the same approach as Arbab¹², using a multi-step procedure to maximize sensitivity. Briefly, single-nucleotide mutation frequencies were tabulated at each target position from sequence alignments in treatment and control data. Treatment data was adjusted for 1) background mutations using untreated control data, 2) sequencing errors, 3) batch effects using other treatment data including published data from Arbab¹², which primarily helped adjust for rare substitution artifacts from library construction. Mutations were then identified that occurred consistently for any editor across replicates to build base editing profiles with sufficient sensitivity to detect rare mutations. Cytosine base editing activity was defined as C to A, G, or T at positions −9 to 20 and G to A or C at positions −9 to 5. For all analysis in this work that required tabulating reads with base editing activity, reads that did not have base editing activity according to these broad profiles were discarded. Window sizes were calculated at 50% or greater efficiency relative to the position-wise maximum.

### Target Library Analysis: Calculating Efficiency and Purity

A minimum of 100 reads was required for calculating editing efficiency, and a minimum of 100 edited reads to calculate purity of editing outcomes. Library members not satisfying these criteria were filtered. The resulting efficiency and purity values were reported as data in the manuscript, and used to train machine learning models. Calculated editing efficiencies and purities were not adjusted for batch effects: instead, the efficiency model is designed to account for batch variation in baseline editing efficiencies by taking it in as optional input. Bystander editing patterns were not found to vary substantially by batch (Arbab).

### Target Library Analysis: Clustering

CGBEs transversion purities at (target site, nucleotide) tuples in the comprehensive context library were tabulated, and pairwise distances between CGBEs were calculated as the variance explained (R2) between each pair of CGBEs. Clustering was performed using the L1 distance metric between vectors with the UPGMA clustering algorithm (average linkage).

Target Library Analysis: Identifying Targets with Diverse Editing Outcomes

A “diversity score” was calculated for a target site and substrate nucleotide given observed editing activity values (yield or purity) by a panel of base editors. For a vector of observed values denoted x, the diversity score was defined as max(x)+2*std(x). Max(x) was included in the score function to encourage library members with very high and very low values to be considered diverse.

To explore the possibility that observed diversity of transversion purity could be explained by analyzing low-abundance outlier library members, the relationship between the diversity of transversion purity and library member abundance in the transversion-enriched SNV library was investigated. A diversity score was calculated for each library member, where large values indicate that different CGBEs had different transversion editing purity at that target. The relative abundance of each library member in the sequencing data was also calculated. If library members with extremely high diversity scores were associated with low relative abundance (e.g., if they were explainable by low coverage bottlenecking outliers), their relative abundances should be shifted relative to the background distribution.

This hypothesis was tested by comparing the distribution of relative abundance for the top 10 to top 50 library members ranked by diversity score to the full distribution of relative abundances. By Welch's T-Test, no statistical evidence that high-diversity library members had shifted relative abundance (P>0.40, N=4,000) was found. Furthermore, a mildly positive Pearson correlation (R=0.14, P=4×10-14) between relative abundance and the diversity score was observed, indicating that across the whole library, library members with higher relative abundance tend to have slightly higher diversity of base editing outcomes. Taken together with other analysis presented herein, it is concluded that differences in editing purity by different CGBEs at the same target are better explained by their distinct sequence preferences.

### Target Library Analysis: Sequence Motif Models

For prediction tasks where the target variable is continuous and has range in (0, 1), a logistic transformation was first applied to the data, then linear regression was used. For continuous data representing fractions, values equal to 0 or 1 were discarded. For classification tasks, the target variables were either 0 or 1 indicating absence or presence of activity, and logistic regression was used. Target variables included the efficiency of C•G-to-T•A editing by CBEs and the purity of cytosine transversions by CBEs. Each of these statistics involves calculating a denominator corresponding to the total number of reads at a target sequence, or the total number of edited reads at a target sequence not including indels. Target sequences with fewer than 100 reads in the denominator were discarded to ensure the accuracy of estimated statistics in the training and testing data. Features were obtained by one-hot-encoding nucleotides per position relative to a substrate nucleotide or to the protospacer. When featurizing data relative to a single substrate nucleotide, each substrate nucleotide within a specified range of positions was used. Ranges used included position 6 only (for the comprehensive context library that contained all NNN-NNN-mers surrounding position 6) and positions 4-8, which was used only when exploratory data analysis indicated that the activity of interest did not vary substantially by position. All nucleotides within a 10-bp radius of the target position were one-hot-encoded. Position was not used as a feature. The data were randomly split into training and test sets at an 80:20 ratio. It is noted that sequence motifs described by these regression models consider each position independently and are intended primarily for visualization.

Motifs for yield were calculated from the top 150 cytosines ranked by C-to-G yield. Column sizes are scaled by their information content.

### Target Library Analysis: Base Editing Efficiency Models

It was observed that base editing efficiency varies by experimental batch. To combine replicates across batches, mean centering and logit transformation was first performed at up to 10,638 gRNA-target pairs in each experimental condition separately from the 12kChar library which includes all 4-mers surrounding A or C from protospacer positions 1 to 11. Data at target sites with fewer than 100 total reads were discarded, then values were averaged at matched target sites across experimental replicates. Values of negative or positive infinity (resulting from logit of 0 or 1) were discarded. The data were randomly split into training and test sets at a ratio of 90:10. Each target site had a single output value corresponding to the mean logit fraction of sequenced reads with any base editing activity. Data points comprising a single replicate were assigned weight=0.5. Data points comprising multiple replicates were assigned a weight of the median logit variance divided by the logit variance at that data point, or 1, whichever value was smaller. In this manner, exactly half of the data points comprising multiple replicates were assigned a weight of 1, and those with higher variance were assigned a lower weight. Features were obtained from each target sequence using protospacer positions −9 to 21. Features included one-hot encoded single nucleotide identities at each position, one-hot encoded dinucleotides at neighboring positions, the melting temperature of the sequence and various subsequences, the total number of each nucleotide in the sequence, and the total number of G or C nucleotides in the sequence.

Gradient-boosted regression trees from the python package scikit-learn were used and trained with tuples of (x, y, weights) using the training data. Hyperparameter optimization was performed as described in Arbab⁸. 5-fold cross-validation was performed by splitting the training set into a training and validation set at a ratio of 8:1 and retained the combination of hyperparameters with the strongest average cross-validation performance as the final model. Models were trained in this manner for each combination of cell-type and base editor. Models were evaluated on the test set which was not used during hyperparameter optimization.

### Target Library Analysis: Bystander Editing Models

Bystander models were designed and trained using the same approach as Arbab. Briefly, a deep conditional autoregressive model that uses an input target sequence surrounding a protospacer and PAM to output a frequency distribution on combinations of base editing outcomes in the python package PyTorch28 was designed and implemented. The model predicts substitutions at cytosines and guanines for CBEs. The model transforms each substrate nucleotide and its local context using a shared encoder into a deep representation, then applies an autoregressive decoder that iteratively generates a distribution over base editing outcomes at each substrate nucleotide while conditioning on all previous generated outcomes. The encoder and decoder are coupled with a learned position-wise bias towards producing an unedited outcome. The model is trained on observed data by minimizing the KL divergence. Importantly, the conditional autoregressive design is sufficiently expressive to learn any possible joint distribution in the output space, thereby representing a powerful and general method for learning the editing tendencies of any base editor from data. A dataset was assembled where each sgRNA-target pair was matched with a table of observed base editing genotypes and their frequencies among reads with edited outcomes. Data points with fewer than 100 edited reads were discarded. Edited genotypes occurring at higher than 2.5% frequency with no edits at any substrate nucleotides (defined as C for CBEs and A for ABEs) in positions 1-10 were discarded. Data from multiple experimental replicates were combined by summing read counts for each observed genotype.

### Target Library Analysis: Performance Evaluation

Machine learning model performance was evaluated using held-out data. For evaluating models at predicting yield, the efficiency model was used to predict a base editing efficiency score using efficiency summary statistics (mean, std) from the training set. The predicted base editing efficiency with the predicted frequency of editing patterns was multiplied from the bystander model.

### Target Library Analysis: Indel Quantification

Indels were quantified using the same approach as Arbab⁸. Indels have strong batch effects in the library assay which can be adjusted within each connected component in the graph defined with nodes representing base editors and edges connecting base editors measured in the same experimental batch. Batch effects for eA3A-nCas9 were adjusted using two-way ANOVA as previously described since it was included in the same connected component as all BEs previously characterized in Arbab⁸. Batch effects for all other CGBEs were not able to be adjusted as they were in a separate connected component.

CGBEs are expected to generate indels at higher frequency than canonical base editors as a consequence of generating abasic sites more efficiently. Consistent with this expectation, it was previously observed lower base editing to indel (BE:indel) ratios at sites with higher transversion base editing activity. However, surprisingly, a positive correlation between BE:indel ratios and high C•G-to-G•C editing purity was observed among target library editing outcomes. The geometric mean BE:indel ratio for eA3A-nCas9 was 15:1 across all target sequences, lower than canonical CBEs at 40:18; however, upon close inspection, it was recognized that BE:indel ratios were split dependent upon whether the target sequence was edited with high or low purity. Indeed, the geometric mean BE:indel ratio was below this 15:1 ratio for sites with <40% C•G-to-G•C purity (decreases from 17:1 to 12:1 as editing purity increases from 0% to 40%) while the geometric average BE:indel ratio increased from 12:1 to 29:1 as C•G-to-G•C purity increased from 40% to 100%. This surprising positive correlation between BE:indel ratios and C•G-to-G•C purity was observed for 11 CGBEs across the comprehensive context and transversion-enriched libraries, with R=0.05 to 0.20 (P<2.4×10-6). No CGBE had a statistically significant negative correlation. This observation suggests that while abasic sites are a common precursor of both indel formation and C•G-to-G•C substitutions and that increased abasic site formation should lead to increases in both indels and C•G-to-G•C substitutions, target sites particularly amenable to highly pure C•G-to-G•C editing preferentially resolve abasic sites against indels. Taken together, these observations highlight the possibility of developing CGBEs with both highly pure C•G-to-G•C editing and high BE:indel ratios.

### Target Library Analysis: Evaluating CGBE-Hive Optimization of CGBEs for SNVs

Six CGBEs were used for this analysis: Anc689-nCas9-NG, APOBEC1-nCas9-NG, and eA3A-nCas9-NG, UdgX-Anc689-UdgX-nCas9-NG-RBMX, UdgX-APOBEC1-UdgX-nCas9-NG, and UdgX-APOBEC1-UdgX-HF-nCas9-NG. For each SNV, CGBE-Hive was used to identify which CGBE had the highest predicted genotype correction precision or amino acid correction precision among CGBEs that had data for that SNV, which was not always all six CGBEs, as some conditions had different SNVs filtered out due to low read counts or poor data quality. Only SNVs with data for at least three CGBEs were considered. The baseline used was the expectation of the statistic with respect to a uniform distribution over the six CGBEs for each SNV.

### Obtaining Biological Materials

Plasmids encoding CGBEs and CRISPRi screening materials are available through Addgene.

TABLE 5
Prime Editing oligonucleotides

| | |
|---|---|
| Name | Sequence |

pegRNA scaffold oligos (SEQ ID NOs: 232-233)

| | |
|---|---|
| pegRNA_scaffold_top | 5′phos- |
| | AGAGCTAGAAATAGCAAGTTAAAATAAGGCTAGTCCGTTATCAACTTGAAAAAGT |
| | GGCACCGAGTCG |
| pegRNA_scaffold_bottom | 5′phos- |
| | GCACCGACTCGGTGCCACTTTTTCAAGTTGATAACGGACTAGCCTTATTTTAACTTG |
| | CTATTTCTAG |

pegRNA spacer oligos (SEQ ID NOs: 234-245)

| | |
|---|---|
| HEK3_+1GtoC_spacer_top | caccGCTGCCATCACGTGCTCAGTCgtttt |
| HEK3_+1GtoC_spacer_bottom | ctctaaaacGACTGAGCACGTGATGGCAGC |
| HEK3_+13GtoC_spacer_top | caccGCTGGCCTGGGTCAATCCTTGgtttt |
| HEK3_+13GtoC_spacer_bottom | ctctaaaacCAAGGATTGACCCAGGCCAGC |
| FANCF_+5GtoC_spacer_top | caccGAGCGATCCAGGTGCTGCAGAgtttt |
| FANCF_+5GtoC_spacer_bottom | ctctaaaacTCTGCAGCACCTGGATCGCTC |
| RNF2_+14GtoC_spacer_top | caccGTACACGTCTCATATGCCCCTgtttt |
| RNF2_+14GtoC_spacer_bottom | ctctaaaacAGGGGCATATGAGACGTGTAC |
| HBB_+3GtoC_spacer_top | caccGTGCACCATGGTGTCTGTTTGgtttt |
| HBB_+3GtoC_spacer_bottom | ctctaaaacCAAACAGACACCATGGTGCAC |
| HBB_+16GtoC_spacer_top | caccGCTCAGGAGTCAGGTGCACCAgtttt |
| HBB_+16GtoC_spacer_bottom | ctctaaaacTGGTGCACCTGACTCCTGAGC |

sgRNA spacer oligos (SEQ ID NOs: 246-279)

| FANCF_nickA_top | caccGGAATCCGTTCTGCAGCACC | | |
|---|---|---|---|
| FANCF_nickA_bottom | aaacGGTGCTGCAGAACGGATTCC | | |
| FANCF_nickB_top | caccGCGCCGTCTCCAAGGTGAAAG | | |
| FANCF_nickB_bottom | aaacCTTTCACCTTGGAGACGGCGC | | |
| FANCF_nickC_top | caccGCAGAGAGTCGCCGTCTCCA | | |
| FANCF_nickC_bottom | aaacTGGAGACGGCGACTCTCTGC | | |
| FANCF_nickD_top | caccGCAGAGAGGCGTATCATTTCG | | |
| FANCF_nickD_bottom | aaacCGAAATGATACGCCTCTCTGC | | |
| HBB_nick2_top | caccGGGCTGGGCATAAAAGTCA | | |
| HBB_nick2_bottom | aaacTGACTTTTATGCCCAGCCC | | |
| HBB_nick3_top | caccGGAGGGCAGGAGCCAGGGCT | | |
| HBB_nick3_bottom | aaacAGCCCTGGCTCCTGCCCTCC | | |
| HBB_nickA_top | caccGCAACCTGAAACAGACACCA | | |
| HBB_nickA_bottom | aaacTGGTGTCTGTTTCAGGTTGC | | |
| HEK3_nick2_top | caccGACGCCCTCTGGAGGAAGCA | | |
| HEK3_nick2_bottom | aaacTGCTTCCTCCAGAGGGCGTC | | |
| HEK3_nick3_top | caccGCTGTCCTGCGACGCCCTC | | |
| HEK3_nick3_bottom | aaacGAGGGCGTCGCAGGACAGC | | |
| HEK3_nick5_top | caccGCACATACTAGCCCCTGTCT | | |
| HEK3_nick5_bottom | aaacAGACAGGGGCTAGTATGTGC | | |
| HEK3_nick6_top | caccGTCAACCAGTATCCCGGTGC | | |
| HEK3_nick6_bottom | aaacGCACCGGGATACTGGTTGAC | | |
| HEK3_nickA_top | caccGCAAGTAAGCATGCATTTGT | | |
| HEK3_nickA_bottom | aaacACAAATGCATGCTTACTTGC | | |
| HEK3_nickB_top | caccGGCCCAGAGTGAGCACGTGA | | |
| HEK3_nickB_bottom | aaacTCACGTGCTCACTCTGGGCC | | |
| HEK3_nickC_top | caccGCTGCCATCACGTGCTCACTC | | |
| HEK3_nickC_bottom | aaacGAGTGAGCACGTGATGGCAGC | | |
| RNF2_nick1_top | caccGTCAACCATTAAGCAAAACAT | | |
| RNF2_nick1_bottom | aaacATGTTTTGCTTAATGGTTGAC | | |
| RNF2_nick2_top | caccGTCTCAGGCTGTGCAGACAAA | | |
| RNF2_nick2_bottom | aaacTTTGTCTGCACAGCCTGAGAC | | |
| RNF2_nickA_top | caccGAATGACTAACATGACTGCCA | | |
| RNF2_nickA_bottom | aaacTGGCAGTCATGTTAGTCATTC | | |
| Name | Sequence | PBA | template |
| HEK3_+1GtoC_1_top | gtgcGGGCCCAGAGTGAGCACGT | 9 | 10 |
| HEK3_+1GtoC_1_bottom | aaaaACGTGCTCACTCTGGGCCC | 9 | 10 |
| HEK3_+1GtoC_2_top | gtgcTTGGGGCCCAGAGTGAGCACGT | 9 | 13 |
| HEK3_+1GtoC_2_bottom | aaaaACGTGCTCACTCTGGGCCCCAA | 9 | 13 |
| HEK3_+1GtoC_3_top | gtgcTCCTTGGGGCCCAGAGTGAGCACGT | 9 | 16 |
| HEK3_+1GtoC_3_bottom | aaaaACGTGCTCACTCTGGGCCCCAAGGA | 9 | 16 |
| HEK3_+1GtoC_4_top | gtgcAATCCTTGGGGCCCAGAGTGAGCACGT | 9 | 18 |
| HEK3_+1GtoC_4_bottom | aaaaACGTGCTCACTCTGGGCCCCAAGGATT | 9 | 18 |
| HEK3_+1GtoC_5_top | gtgcGGGCCCAGAGTGAGCACGTG | 10 | 10 |
| HEK3_+1GtoC_5_bottom | aaaaCACGTGCTCACTCTGGGCCC | 10 | 10 |
| HEK3_+1GtoC_6_top | gtgcTTGGGGCCCAGAGTGAGCACGTG | 10 | 13 |
| HEK3_+1GtoC_6_bottom | aaaaCACGTGCTCACTCTGGGCCCCAA | 10 | 13 |
| HEK3_+1GtoC_7_top | gtgcTCCTTGGGGCCCAGAGTGAGCACGTG | 10 | 16 |
| HEK3_+1GtoC_7_bottom | aaaaCACGTGCTCACTCTGGGCCCCAAGGA | 10 | 16 |
| HEK3_+1GtoC_8_top | gtgcAATCCTTGGGGCCCAGAGTGAGCACGTG | 10 | 18 |
| HEK3_+1GtoC_8_bottom | aaaaCACGTGCTCACTCTGGGCCCCAAGGATT | 10 | 18 |
| HEK3_+1GtoC_9_top | gtgcGGGCCCAGAGTGAGCACGTGAT | 12 | 10 |
| HEK3_+1GtoC_9_bottom | aaaaATCACGTGCTCACTCTGGGCCC | 12 | 10 |
| HEK3_+1GtoC_10_top | gtgcTTGGGGCCCAGAGTGAGCACGTGAT | 12 | 13 |
| HEK3_+1GtoC_10_bottom | aaaaATCACGTGCTCACTCTGGGCCCCAA | 12 | 13 |
| HEK3_+1GtoC_11_top | gtgcTCCTTGGGGCCCAGAGTGAGCACGTGAT | 12 | 16 |
| HEK3_+1GtoC_11_bottom | aaaaATCACGTGCTCACTCTGGGCCCCAAGGA | 12 | 16 |
| HEK3_+1GtoC_12_top | gtgcAATCCTTGGGGCCCAGAGTGAGCACGTGAT | 12 | 18 |
| HEK3_+1GtoC_12_bottom | aaaaATCACGTGCTCACTCTGGGCCCCAAGGATT | 12 | 18 |
| HEK3_+1GtoC_13_top | gtgcGGGCCCAGAGTGAGCACGTGATG | 13 | 10 |
| HEK3_+1GtoC_13_bottom | aaaaCATCACGTGCTCACTCTGGGCCC | 13 | 10 |
| HEK3_+1GtoC_14_top | gtgcTTGGGGCCCAGAGTGAGCACGTGATG | 13 | 13 |
| HEK3_+1GtoC_14_bottom | aaaaCATCACGTGCTCACTCTGGGCCCCAA | 13 | 13 |
| HEK3_+1GtoC_15_top | gtgcTCCTTGGGGCCCAGAGTGAGCACGTGATG | 13 | 16 |
| HEK3_+1GtoC_15_bottom | aaaaCATCACGTGCTCACTCTGGGCCCCAAGGA | 13 | 16 |
| HEK3_+1GtoC_16_top | gtgcAATCCTTGGGGCCCAGAGTGAGCACGTGATG | 13 | 18 |
| HEK3_+1GtoC_16_bottom | aaaaCATCACGTGCTCACTCTGGGCCCCAAGGATT | 13 | 18 |
| HEK3_+13GtoC_1_top | gtgcGTGCTCACTCTGGGCCCCAAGGATTGACC | 9 | 20 |
| HEK3_+13GtoC_1_bottom | aaaaGGTCAATCCTTGGGGCCCAGAGTGAGCAC | 9 | 20 |
| HEK3_+13GtoC_2_top | gtgcACGTGCTCACTCTGGGCCCCAAGGATTGACC | 9 | 22 |
| HEK3_+13GtoC_2_bottom | aaaaGGTCAATCCTTGGGGCCCAGAGTGAGCACGT | 9 | 22 |
| HEK3_+13GtoC_3_top | gtgcATCACGTGCTCACTCTGGGCCCCAAGGATTGACC | 9 | 25 |
| HEK3_+13GtoC_3_bottom | aaaaGGTCAATCCTTGGGGCCCAGAGTGAGCACGTGAT | 9 | 25 |
| HEK3_+13GtoC_4_top | gtgcGTGCTCACTCTGGGCCCCAAGGATTGACCCA | 11 | 20 |
| HEK3_+13GtoC_4_bottom | aaaaTGGGTCAATCCTTGGGGCCCAGAGTGAGCAC | 11 | 20 |
| HEK3_+13GtoC_5_top | gtgcACGTGCTCACTCTGGGCCCCAAGGATTGACCCA | 11 | 22 |
| HEK3_+13GtoC_5_bottom | aaaaTGGGTCAATCCTTGGGGCCCAGAGTGAGCACGT | 11 | 22 |
| HEK3_+13GtoC_6_top | gtgcATCACGTGCTCACTCTGGGCCCCAAGGATTGACCCA | 11 | 25 |
| HEK3_+13GtoC_6_bottom | aaaaTGGGTCAATCCTTGGGGCCCAGAGTGAGCACGTGAT | 11 | 25 |
| HEK3_+13GtoC_7_top | gtgcGTGCTCACTCTGGGCCCCAAGGATTGACCCAG | 12 | 20 |
| HEK3_+13GtoC_7_bottom | aaaaCTGGGTCAATCCTTGGGGCCCAGAGTGAGCAC | 12 | 20 |
| HEK3_+13GtoC_8_top | gtgcACGTGCTCACTCTGGGCCCCAAGGATTGACCCAG | 12 | 22 |
| HEK3_+13GtoC_8_bottom | aaaaCTGGGTCAATCCTTGGGGCCCAGAGTGAGCACGT | 12 | 22 |
| HEK3_+13GtoC_9_top | gtgcATCACGTGCTCACTCTGGGCCCCAAGGATTGACCCAG | 12 | 25 |
| HEK3_+13GtoC_9_bottom | aaaaCTGGGTCAATCCTTGGGGCCCAGAGTGAGCACGTGAT | 12 | 25 |
| HEK3_+13GtoC_10_top | gtgcGTGCTCACTCTGGGCCCCAAGGATTGACCCAGG | 13 | 20 |
| HEK3_+13GtoC_10_bottom | aaaaCCTGGGTCAATCCTTGGGGCCCAGAGTGAGCAC | 13 | 20 |
| HEK3_+13GtoC_11_top | gtgcACGTGCTCACTCTGGGCCCCAAGGATTGACCCAGG | 13 | 22 |
| HEK3_+13GtoC_11_bottom | aaaaCCTGGGTCAATCCTTGGGGCCCAGAGTGAGCACGT | 13 | 22 |
| HEK3_+13GtoC_12_top | gtgcATCACGTGCTCACTCTGGGCCCCAAGGATTGACCCAGG | 13 | 25 |
| HEK3_+13GtoC_12_bottom | aaaaCCTGGGTCAATCCTTGGGGCCCAGAGTGAGCACGTGAT | 13 | 25 |
| FANCF_+5GtoC_1_top | gtgcGAATCCGTTCTGCAGCACCT | 9 | 11 |
| FANCF_+5GtoC_1_bottom | aaaaAGGTGCTGCAGAACGGATTC | 9 | 11 |
| FANCF_+5GtoC_2_top | gtgcATGGAATCCGTTCTGCAGCACCT | 9 | 14 |
| FANCF_+5GtoC_2_bottom | aaaaAGGTGCTGCAGAACGGATTCCAT | 9 | 14 |
| FANCF_+5GtoC_3_top | gtgcTCATGGAATCCGTTCTGCAGCACCT | 9 | 16 |
| FANCF_+5GtoC_3_bottom | aaaaAGGTGCTGCAGAACGGATTCCATGA | 9 | 16 |
| FANCF_+5GtoC_4_top | gtgcACCTCATGGAATCCGTTCTGCAGCACCT | 9 | 19 |
| FANCF_+5GtoC_4_bottom | aaaaAGGTGCTGCAGAACGGATTCCATGAGGT | 9 | 19 |
| FANCF_+5GtoC_5_top | gtgcGAATCCGTTCTGCAGCACCTG | 10 | 11 |
| FANCF_+5GtoC_5_bottom | aaaaCAGGTGCTGCAGAACGGATTC | 10 | 11 |
| FANCF_+5GtoC_6_top | gtgcATGGAATCCGTTCTGCAGCACCTG | 10 | 14 |
| FANCF_+5GtoC_6_bottom | aaaaCAGGTGCTGCAGAACGGATTCCAT | 10 | 14 |
| FANCF_+5GtoC_7_top | gtgcTCATGGAATCCGTTCTGCAGCACCTG | 10 | 16 |
| FANCF_+5GtoC_7_bottom | aaaaCAGGTGCTGCAGAACGGATTCCATGA | 10 | 16 |
| FANCF_+5GtoC_8_top | gtgcACCTCATGGAATCCGTTCTGCAGCACCTG | 10 | 19 |
| FANCF_+5GtoC_8_bottom | aaaaCAGGTGCTGCAGAACGGATTCCATGAGGT | 10 | 19 |
| FANCF_+5GtoC_9_top | gtgcGAATCCGTTCTGCAGCACCTGG | 11 | 11 |
| FANCF_+5GtoC_9_bottom | aaaaCCAGGTGCTGCAGAACGGATTC | 11 | 11 |
| FANCF_+5GtoC_10_top | gtgcATGGAATCCGTTCTGCAGCACCTGG | 11 | 14 |
| FANCF_+5GtoC_10_bottom | aaaaCCAGGTGCTGCAGAACGGATTCCAT | 11 | 14 |
| FANCF_+5GtoC_11_top | gtgcTCATGGAATCCGTTCTGCAGCACCTGG | 11 | 16 |
| FANCF_+5GtoC_11_bottom | aaaaCCAGGTGCTGCAGAACGGATTCCATGA | 11 | 16 |
| FANCF_+5GtoC_12_top | gtgcACCTCATGGAATCCGTTCTGCAGCACCTGG | 11 | 19 |
| FANCF_+5GtoC_12_bottom | aaaaCCAGGTGCTGCAGAACGGATTCCATGAGGT | 11 | 19 |
| FANCF_+5GtoC_13_top | gtgcGAATCCGTTCTGCAGCACCTGGAT | 13 | 11 |
| FANCF_+5GtoC_13_bottom | aaaaATCCAGGTGCTGCAGAACGGATTC | 13 | 11 |
| FANCF_+5GtoC_14_top | gtgcATGGAATCCGTTCTGCAGCACCTGGAT | 13 | 14 |
| FANCF_+5GtoC_14_bottom | aaaaATCCAGGTGCTGCAGAACGGATTCCAT | 13 | 14 |
| FANCF_+5GtoC_15_top | gtgcTCATGGAATCCGTTCTGCAGCACCTGGAT | 13 | 16 |
| FANCF_+5GtoC_15_bottom | aaaaATCCAGGTGCTGCAGAACGGATTCCATGA | 13 | 16 |
| FANCF_+5GtoC_16_top | gtgcACCTCATGGAATCCGTTCTGCAGCACCTGGAT | 13 | 19 |
| FANCF_+5GtoC_16_bottom | aaaaATCCAGGTGCTGCAGAACGGATTCCATGAGGT | 13 | 19 |
| RNF2_+14GtoC_1_top | gtgcGACTAACATGACTGCCAAGGGGCATATGA | 9 | 20 |
| RNF2_+14GtoC_1_bottom | aaaaTCATATGCCCCTTGGCAGTCATGTTAGTC | 9 | 20 |
| RNF2_+14GtoC_2_top | gtgcAATGACTAACATGACTGCCAAGGGGCATATGA | 9 | 23 |
| RNF2_+14GtoC_2_bottom | aaaaTCATATGCCCCTTGGCAGTCATGTTAGTCATT | 9 | 23 |
| RNF2_+14GtoC_3_top | gtgcGGTAATGACTAACATGACTGCCAAGGGGCATATGA | 9 | 26 |
| RNF2_+14GtoC_3_bottom | aaaaTCATATGCCCCTTGGCAGTCATGTTAGTCATTACC | 9 | 26 |
| RNF2_+14GtoC_4_top | gtgcTCAGGTAATGACTAACATGACTGCCAAGGGGCATATGA | 9 | 29 |
| RNF2_+14GtoC_4_bottom | aaaaTCATATGCCCCTTGGCAGTCATGTTAGTCATTACCTGA | 9 | 29 |
| RNF2_+14GtoC_5_top | gtgcGACTAACATGACTGCCAAGGGGCATATGAG | 10 | 20 |
| RNF2_+14GtoC_5_bottom | aaaaCTCATATGCCCCTTGGCAGTCATGTTAGTC | 10 | 20 |
| RNF2_+14GtoC_6_top | gtgcAATGACTAACATGACTGCCAAGGGGCATATGAG | 10 | 23 |
| RNF2_+14GtoC_6_bottom | aaaaCTCATATGCCCCTTGGCAGTCATGTTAGTCATT | 10 | 23 |
| RNF2_+14GtoC_7_top | gtgcGGTAATGACTAACATGACTGCCAAGGGGCATATGAG | 10 | 26 |
| RNF2_+14GtoC_7_bottom | aaaaCTCATATGCCCCTTGGCAGTCATGTTAGTCATTACC | 10 | 26 |
| RNF2_+14GtoC_8_top | gtgcTCAGGTAATGACTAACATGACTGCCAAGGGGCATATGAG | 10 | 29 |
| RNF2_+14GtoC_8_bottom | aaaaCTCATATGCCCCTTGGCAGTCATGTTAGTCATTACCTGA | 10 | 29 |
| RNF2_+14GtoC_9_top | gtgcGACTAACATGACTGCCAAGGGGCATATGAGAC | 12 | 20 |
| RNF2_+14GtoC_9_bottom | aaaaGTCTCATATGCCCCTTGGCAGTCATGTTAGTC | 12 | 20 |
| RNF2_+14GtoC_10_top | gtgcAATGACTAACATGACTGCCAAGGGGCATATGAGAC | 12 | 23 |
| RNF2_+14GtoC_10_bottom | aaaaGTCTCATATGCCCCTTGGCAGTCATGTTAGTCATT | 12 | 23 |
| RNF2_+14GtoC_11_top | gtgcGGTAATGACTAACATGACTGCCAAGGGGCATATGAGAC | 12 | 26 |
| RNF2_+14GtoC_11_bottom | aaaaGTCTCATATGCCCCTTGGCAGTCATGTTAGTCATTACC | 12 | 26 |
| RNF2_+14GtoC_12_top | gtgcTCAGGTAATGACTAACATGACTGCCAAGGGGCATATGAGA | 12 | 29 |
| | C | | |
| RNF2_+14GtoC_12_bottom | aaaaGTCTCATATGCCCCTTGGCAGTCATGTTAGTCATTACCTGA | 12 | 29 |
| RNF2_+14GtoC_13_top | gtgcGACTAACATGACTGCCAAGGGGCATATGAGACGT | 14 | 20 |
| RNF2_+14GtoC_13_bottom | aaaaACGTCTCATATGCCCCTTGGCAGTCATGTTAGTC | 14 | 20 |
| RNF2_+14GtoC_14_top | gtgcAATGACTAACATGACTGCCAAGGGGCATATGAGACGT | 14 | 23 |
| RNF2_+14GtoC_14_bottom | aaaaACGTCTCATATGCCCCTTGGCAGTCATGTTAGTCATT | 14 | 23 |
| RNF2_+14GtoC_15_top | gtgcGGTAATGACTAACATGACTGCCAAGGGGCATATGAGACGT | 14 | 26 |
| RNF2_+14GtoC_15_bottom | aaaaACGTCTCATATGCCCCTTGGCAGTCATGTTAGTCATTACC | 14 | 26 |
| RNF2_+14GtoC_16_top | gtgcTCAGGTAATGACTAACATGACTGCCAAGGGGCATATGAGA | 14 | 29 |
| | CGT | | |
| RNF2_+14GtoC_16_bottom | aaaaACGTCTCATATGCCCCTTGGCAGTCATGTTAGTCATTACCT | 14 | 29 |
| | GA | | |
| HBB_+3GtoC_1_top | gtgcGCAACCTGAAACAGACACC | 9 | 10 |
| HBB_+3GtoC_1_bottom | aaaaGGTGTCTGTTTCAGGTTGC | 9 | 10 |
| HBB_+3GtoC_2_top | gtgcTAGCAACCTGAAACAGACACC | 9 | 12 |
| HBB_+3GtoC_2_bottom | aaaaGGTGTCTGTTTCAGGTTGCTA | 9 | 12 |
| HBB_+3GtoC_3_top | gtgcACTAGCAACCTGAAACAGACACC | 9 | 14 |
| HBB_+3GtoC_3_bottom | aaaaGGTGTCTGTTTCAGGTTGCTAGT | 9 | 14 |
| HBB_+3GtoC_4_top | gtgcGTTCACTAGCAACCTGAAACAGACACC | 9 | 18 |
| HBB_+3GtoC_4_bottom | aaaaGGTGTCTGTTTCAGGTTGCTAGTGAAC | 9 | 18 |
| HBB_+3GtoC_5_top | gtgcGCAACCTGAAACAGACACCAT | 11 | 10 |
| HBB_+3GtoC_5_bottom | aaaaATGGTGTCTGTTTCAGGTTGC | 11 | 10 |
| HBB_+3GtoC_6_top | gtgcTAGCAACCTGAAACAGACACCAT | 11 | 12 |
| HBB_+3GtoC_6_bottom | aaaaATGGTGTCTGTTTCAGGTTGCTA | 11 | 12 |
| HBB_+3GtoC_7_top | gtgcACTAGCAACCTGAAACAGACACCAT | 11 | 14 |
| HBB_+3GtoC_7_bottom | aaaaATGGTGTCTGTTTCAGGTTGCTAGT | 11 | 14 |
| HBB_+3GtoC_8_top | gtgcGTTCACTAGCAACCTGAAACAGACACCAT | 11 | 18 |
| HBB_+3GtoC_8_bottom | aaaaATGGTGTCTGTTTCAGGTTGCTAGTGAAC | 11 | 18 |
| HBB_+3GtoC_9_top | gtgcGCAACCTGAAACAGACACCATG | 12 | 10 |
| HBB_+3GtoC_9_bottom | aaaaCATGGTGTCTGTTTCAGGTTGC | 12 | 10 |
| HBB_+3GtoC_10_top | gtgcTAGCAACCTGAAACAGACACCATG | 12 | 12 |
| HBB_+3GtoC_10_bottom | aaaaCATGGTGTCTGTTTCAGGTTGCTA | 12 | 12 |
| HBB_+3GtoC_11_top | gtgcACTAGCAACCTGAAACAGACACCATG | 12 | 14 |
| HBB_+3GtoC_11_bottom | aaaaCATGGTGTCTGTTTCAGGTTGCTAGT | 12 | 14 |
| HBB_+3GtoC_12_top | gtgcGTTCACTAGCAACCTGAAACAGACACCATG | 12 | 18 |
| HBB_+3GtoC_12_bottom | aaaaCATGGTGTCTGTTTCAGGTTGCTAGTGAAC | 12 | 18 |
| HBB_+3GtoC_13_top | gtgcGCAACCTGAAACAGACACCATGG | 13 | 10 |
| HBB_+3GtoC_13_bottom | aaaaCCATGGTGTCTGTTTCAGGTTGC | 13 | 10 |
| HBB_+3GtoC_14_top | gtgcTAGCAACCTGAAACAGACACCATGG | 13 | 12 |
| HBB_+3GtoC_14_bottom | aaaaCCATGGTGTCTGTTTCAGGTTGCTA | 13 | 12 |
| HBB_+3GtoC_15_top | gtgcACTAGCAACCTGAAACAGACACCATGG | 13 | 14 |
| HBB_+3GtoC_15_bottom | aaaaCCATGGTGTCTGTTTCAGGTTGCTAGT | 13 | 14 |
| HBB_+3GtoC_16_top | gtgcGTTCACTAGCAACCTGAAACAGACACCATGG | 13 | 18 |
| HBB_+3GtoC_16_bottom | aaaaCCATGGTGTCTGTTTCAGGTTGCTAGTGAAC | 13 | 18 |
| HBB_+16GtoC_3_top | gtgcTAGCAACCTGAAACAGACACCATGGTGCACCTGA | 9 | 25 |
| HBB_+16GtoC_3_bottom | aaaaTCAGGTGCACCATGGTGTCTGTTTCAGGTTGCTA | 9 | 25 |
| HBB_+16GtoC_4_top | gtgcACTAGCAACCTGAAACAGACACCATGGTGCACCTGA | 9 | 27 |
| HBB_+16GtoC_4_bottom | aaaaTCAGGTGCACCATGGTGTCTGTTTCAGGTTGCTAGT | 9 | 27 |
| HBB_+16GtoC_5_top | gtgcTTCACTAGCAACCTGAAACAGACACCATGGTGCACCTGA | 9 | 30 |
| HBB_+16GtoC_5_bottom | aaaaTCAGGTGCACCATGGTGTCTGTTTCAGGTTGCTAGTGAA | 9 | 30 |
| HBB_+16GtoC_6_top | gtgcAACCTGAAACAGACACCATGGTGCACCTGAC | 10 | 21 |
| HBB_+16GtoC_6_bottom | aaaaGTCAGGTGCACCATGGTGTCTGTTTCAGGTT | 10 | 21 |
| HBB_+16GtoC_7_top | gtgcAGCAACCTGAAACAGACACCATGGTGCACCTGAC | 10 | 24 |
| HBB_+16GtoC_7_bottom | aaaaGTCAGGTGCACCATGGTGTCTGTTTCAGGTTGCT | 10 | 24 |
| HBB_+16GtoC_8_top | gtgcTAGCAACCTGAAACAGACACCATGGTGCACCTGAC | 10 | 25 |
| HBB_+16GtoC_8_bottom | aaaaGTCAGGTGCACCATGGTGTCTGTTTCAGGTTGCTA | 10 | 25 |
| HBB_+16GtoC_9_top | gtgcACTAGCAACCTGAAACAGACACCATGGTGCACCTGAC | 10 | 27 |
| HBB_+16GtoC_9_bottom | aaaaGTCAGGTGCACCATGGTGTCTGTTTCAGGTTGCTAGT | 10 | 27 |
| HBB_+16GtoC_10_top | gtgcTTCACTAGCAACCTGAAACAGACACCATGGTGCACCTGAC | 10 | 30 |
| HBB_+16GtoC_10_bottom | aaaaGTCAGGTGCACCATGGTGTCTGTTTCAGGTTGCTAGTGAA | 10 | 30 |
| HBB_+16GtoC_12_top | gtgcAGCAACCTGAAACAGACACCATGGTGCACCTGACT | 11 | 24 |
| HBB_+16GtoC_12_bottom | aaaaAGTCAGGTGCACCATGGTGTCTGTTTCAGGTTGCT | 11 | 24 |
| HBB_+16GtoC_14_top | gtgcACTAGCAACCTGAAACAGACACCATGGTGCACCTGACT | 11 | 27 |
| HBB_+16GtoC_14_bottom | aaaaAGTCAGGTGCACCATGGTGTCTGTTTCAGGTTGCTAGT | 11 | 27 |
| HBB_+16GtoC_17_top | gtgcAGCAACCTGAAACAGACACCATGGTGCACCTGACTC | 12 | 24 |
| HBB_+16GtoC_17_bottom | aaaaGAGTCAGGTGCACCATGGTGTCTGTTTCAGGTTGCT | 12 | 24 |
| HBB_+16GtoC_18_top | gtgcTAGCAACCTGAAACAGACACCATGGTGCACCTGACTC | 12 | 25 |
| HBB_+16GtoC_18_bottom | aaaaGAGTCAGGTGCACCATGGTGTCTGTTTCAGGTTGCTA | 12 | 25 |
| HBB_+16GtoC_19_top | gtgcACTAGCAACCTGAAACAGACACCATGGTGCACCTGACTC | 12 | 27 |
| HBB_+16GtoC_19_bottom | aaaaGAGTCAGGTGCACCATGGTGTCTGTTTCAGGTTGCTAGT | 12 | 27 |
| HBB_+16GtoC_20_top | gtgcTTCACTAGCAACCTGAAACAGACACCATGGTGCACCTGAC | 12 | 30 |
| | TC | | |
| HBB_+16GtoC_20_bottom | aaaaGAGTCAGGTGCACCATGGTGTCTGTTTCAGGTTGCTAGTG | 12 | 30 |
| | AA | | |
| HBB_+16GtoC_21_top | gtgcAACCTGAAACAGACACCATGGTGCACCTGACTCC | 13 | 21 |
| HBB_+16GtoC_21_bottom | aaaaGGAGTCAGGTGCACCATGGTGTCTGTTTCAGGTT | 13 | 21 |
| HBB_+16GtoC_22_top | gtgcAGCAACCTGAAACAGACACCATGGTGCACCTGACTCC | 13 | 24 |
| HBB_+16GtoC_22_bottom | aaaaGGAGTCAGGTGCACCATGGTGTCTGTTTCAGGTTGCT | 13 | 24 |
| HBB_+16GtoC_23_top | gtgcTAGCAACCTGAAACAGACACCATGGTGCACCTGACTCC | 13 | 25 |
| HBB_+16GtoC_23_bottom | aaaaGGAGTCAGGTGCACCATGGTGTCTGTTTCAGGTTGCTA | 13 | 25 |
| HBB_+16GtoC_24_top | gtgcACTAGCAACCTGAAACAGACACCATGGTGCACCTGACTCC | 13 | 27 |
| HBB_+16GtoC_24_bottom | aaaaGGAGTCAGGTGCACCATGGTGTCTGTTTCAGGTTGCTAGT | 13 | 27 |
| HBB_+16GtoC_25_top | gtgcTTCACTAGCAACCTGAAACAGACACCATGGTGCACCTGAC | 13 | 30 |
| | TCC | | |
| HBB_+16GtoC_25_bottom | aaaaGGAGTCAGGTGCACCATGGTGTCTGTTTCAGGTTGCTAGT | 13 | 30 |
| | GAA | | |

TABLE 6
On-Targets

| | Protospacer | Top Oligo | Bottom Oligo | HTS primer | HTS primer | |
|---|---|---|---|---|---|---|
| | (SEQ ID | (SEQ ID | (SEQ ID | (SEQ ID | (SEQ ID | |
| | NOs: | NOs: | NOs: | NOs: | NOs: | Amplicon for alignment |
| Site | 505-524) | 525-544) | 545-564) | 565-584) | 585-604) | (SEQ ID NOs: 605-624) |
| HEK2 | GAACACAA | caccGAA | aaacGCAG | ACACTCTT | TGGAGTTC | TGAATGGATTCCTTGGAAACAATGATAACA |
| | AGCATAGA | CACAA | TCTATGC | TCCCTACA | AGACGTGT | AGACCTGGCTGAGCTAACTGTGACAGCATG |
| | CTGC | AGCAT | TTTGTGT | CGACGCTC | GCTCTTCC | TGGTAATTTTCCAGCCCGCTGGCCCTGTAAA |
| | | AGACT | TC | TTCCGATC | GATCTTGA | GGAAACTGGAACACAAAGCATAGACTGCG |
| | | GC | | TNNNNCCA | ATGGATTC | GGGCGGGCCAGCCTGAATAGCTGCAAACAA |
| | | | | GCCCCATC | CTTGGAAA | GTGCAGAATATCTGATGATGTCATACGCAC |
| | | | | TGTCAAAC | CAATGA | AGTTTGACAGATGGGGCTGG |
| | | | | T | | |
| HEK | GGCCCAGA | caccGGC | aaacTCAC | ACACTCTT | TGGAGTTC | ATGTGGGCTGCCTAGAAAGGCATGGATGAG |
| SITE 3 | CTGAGCAC | CCAGA | GTGCTC | TCCCTACA | AGACGTGT | AGAAGCCTGGAGACAGGGATCCCAGGGAAA |
| | GTGA | CTGAG | AGTCTG | CGACGCTC | GCTCTTCC | CGCCCATGCAATTAGTCTATTTCTGCTGCAA |
| | | CACGT | GGCC | TTCCGATC | GATCTCCC | GTAAGCATGCATTTGTAGGCTTGATGCTTTT |
| | | GA | | TNNNNATG | AGCCAAAC | TTTCTGCTTCTCCAGCCCTGGCCTGGGTCAA |
| | | | | TGGGCTGC | TTGTCAAC | TCCTTGGGGCCCAGACTGAGCACGTGATG |
| | | | | CTAGAAAG | C | GCAGAGGAAAGGAAGCCCTGCTTCCTCCAG |
| | | | | G | | AGGGCGTCGCAGGACAGCTTTTCCTAGACA |
| | | | | | | GGGGCTAGTATGTGCAGCTCCTGCACCGGG |
| | | | | | | ATACTGGTTGACAAGTTTGGCTGGG |
| HEK4 | GGCACTGC | caccGGC | aaacCCAC | ACACTCTT | TGGAGTTC | GAACCCAGGTAGCCAGAGACCCGCTGGTCT |
| | GGCTGGAG | ACTGC | CTCCAG | TCCCTACA | AGACGTGT | TCTTTCCCCTCCCCTGCCCTCCCCTCCCTTCA |
| | GTGG | GGCTG | CCGCAG | CGACGCTC | GCTCTTCC | AGATGGCTGACAAAGGCCGGGCTGGGTGGA |
| | | GAGGT | TGCC | TTCCGATC | GATCTTCC | AGGAAGGGAGGAAGGGCGAGGCAGAGGGT |
| | | GG | | TNNNNGA | TTTCAACC | CCAAAGCAGGATGACAGGCAGGGGCACCGC |
| | | | | ACCCAGGT | CGAACGG | GGCGCCCCGGTGGCACTGCGGCTGGAGGT |
| | | | | AGCCAGA | AG | GGGGGTTAAAGCGGAGACTCTGGTGCTGTG |
| | | | | GAC | | TGACTACAGTGGGGGCCCTGCCCTCTCTGAG |
| | | | | | | CCCCCGCCTCCAGGCCTGTGTGTGTGTCTCC |
| | | | | | | GTTCGGGTTGAAAGGA |
| RNF2 | GTCATCTT | caccGTC | aaacCAGG | ACACTCTT | TGGAGTTC | ACGTCTCATATGCCCCTTGGCAGTCATCTTA |
| | AGTCATTA | ATCTTA | TAATGA | TCCCTACA | AGACGTGT | GTCATTACCTGAGGTGTTCGTTGTAACTCA |
| | CCTG | GTCATT | CTAAGA | CGACGCTC | GCTCTTCC | TATAAACTGAGTTCCCATGTTTTGCTTAATG |
| | | ACCTG | TGAC | TTCCGATC | GATCTACG | GTTGAGTTCCGTTTGTCTGCACAGCCTGAGA |
| | | | | TNNNNACG | TAGGAATT | CATTGCTGGAAATAAAGAAGAGAGAAAAAC |
| | | | | TCTCATAT | TTGGTGGG | AATTTTAGTATTTGGAAGGGAAGTGCTATGG |
| | | | | GCCCCTTG | ACA | TCTGAATGTATGTGTCCCACCAAAATTCCTA |
| | | | | G | | CGT |
| EMX1 | GAGTCCGA | caccGAG | aaacTTCT | ACACTCTT | GTGGGTTT | CAGCTCAGCCTGAGTGTTGAGGCCCCAGTG |
| | GCAGAAGA | TCCGA | TCTTCTG | TCCCTACA | TGGAGTTC | GCTGCTCTGGGGGCCTCCTGAGTTTCTCATC |
| | AGAA | GCAGA | CTCGGA | CGACGCTC | AGACGTGT | TGTGCCCCTCCCTCCCTGGCCCAGGTGAAGG |
| | | AGAAG | CTC | TTCCGATC | GCTCTTCC | TGTGGTTCCAGAACCGGAGGACAAAGTACA |
| | | AA | | TNNNNCAG | GATCTCTC | AACGGCAGAAGCTGGAGGAGGAAGGGCCT |
| | | | | CTCAGCCT | GTGGTTGC | GAGTCCGAGCAGAAGAAGAAGGGCTCCCA |
| | | | | GAGTGTTG | | TCACATCAACCGGTGGCGCATTGCCACGAA |
| | | | | A | | GCAGGCCAATGGGGAGGACATCGATGTCAC |
| | | | | | | CTCCAATGACTAGGGTGGGCAACCACAAAC |
| | | | | | | CCACGAG |
| FANCF | GGAATCCC | caccGGA | aaacGGTG | ACACTCTT | TGGAGTTC | GGGGTCCCAGGTGCTGACGTAGGTAGTGCT |
| | TTCTGCAG | ATCCCT | CTGCAG | TCCCTACA | AGACGTGT | TGAGACCGCCAGAAGCTCGGAAAAGCGATC |
| | CACC | TCTGCA | AAGGGA | CGACGCTC | GCTCTTCC | CAGGTGCTGCAGAAGGGATTCCATGAGGT |
| | | GCACC | TTCC | TTCCGATC | GATCTGGG | GCGCGAAGGCCCTACTTCCGCTTTCACCTTG |
| | | | | TGCAGAGA | GTCCCAGG | GAGACGGCGACTCTCTGCGTACTGATTGGA |
| | | | | TNNNNCAT | TGCTGAC | ACATCCGCGAAATGATACGCCTCTCTGCAAT |
| | | | | GGCGTATC | | G |
| | | | | A | | |
| HBBa | GCAACCTC | caccGCA | aaacTGGT | ACACTCTT | TGGAGTTC | AGGGTTGGCCAATCTACTCCCAGGAGCAGG |
| | AAACAGAC | ACCTCA | GTCTGTT | TCCCTACA | AGACGTGT | GAGGGCAGGAGCCAGGGCTGGGCATAAAA |
| | ACCA | AACAG | TGAGGT | CGACGCTC | GCTCTTCC | GTCAGGGCAGAGCCATCTATTGCTTACATTT |
| | | ACACC | TGC | TTCCGATC | GATCTGTC | GCTTCTGACACAACTGTGTTCACTAGCAAC |
| | | A | | TNNNNAG | TTCTCTGT | CTCAAACAGACACCATGGTGCATCTGACTC |
| | | | | GGTTGGCC | CTCCACAT | CTGAGGAGAAGTCTGCCGTTACTGCCCTGTG |
| | | | | AATCTACT | GCC | GGGCAAGGTGAACGTGGATGAAGTTGGTGG |
| | | | | CCC | | TGAGGCCCTGGGCAGGTTGGTATCAAGGTT |
| | | | | | | ACAAGACAGGTTTAAGGAGACCAATAGAAA |
| | | | | | | CTGGGCATGTGGAGACAGAGAAGAC |
| HEK4.1 | gCCTCCAG | caccgCC | aaacGGTG | ACACTCTT | TGGAGTTC | GAACCCAGGTAGCCAGAGACCCGCTGGTCT |
| | CCGCAGTG | TCCAGC | GCACTG | TCCCTACA | AGACGTGT | TCTTTCCCCTCCCCTGCCCTCCCCTCCCTTCA |
| | CCACC | CGCAG | CGGCTG | CGACGCTC | GCTCTTCC | AGATGGCTGACAAAGGCCGGGCTGGGTGGA |
| | | TGCCAC | GAGGc | TTCCGATC | GATCTTCC | AGGAAGGGAGGAAGGGCGAGGCAGAGGGT |
| | | C | | TNNNNGA | TTTCAACC | CCAAAGCAGGATGACAGGCAGGGGCACCGC |
| | | | | ACCCAGGT | CGAACGG | GGCGCCCCGGTGGCACTGCGGCTGGAGGT |
| | | | | AGCCAGA | AG | GGGGGTTAAAGCGGAGACTCTGGTGCTGTG |
| | | | | GAC | | TGACTACAGTGGGGGCCCTGCCCTCTCTGAG |
| | | | | | | CCCCCGCCTCCAGGCCTGTGTGTGTGTCTCC |
| | | | | | | GTTCGGGTTGAAAGGA |
| HEK21 | GCACTTGT | caccGCA | aaacGAAT | ACACTCTT | TGGAGTTC | TGAATGGATTCCTTGGAAACAATGATAACA |
| | TTGCAGCT | CTTGTT | AGCTGC | TCCCTACA | AGACGTGT | AGACCTGGCTGAGCTAACTGTGACAGCATG |
| | ATTC | TGCAG | AAACAA | CGACGCTC | GCTCTTCC | TGGTAATTTTCCAGCCCGCTGGCCCTGTAAA |
| | | CTATTC | GTGC | TTCCGATC | GATCTTGA | GGAAACTGGAACACAAAGCATAGACTGCGG |
| | | | | TNNNNCCA | ATGGATTC | GGCGGGCCAGCCTGAATAGCTGCAAACAA |
| | | | | GCCCCATC | CTTGGAAA | GTGCAGAATATCTGATGATGTCATACGCAC |
| | | | | TGTCAAAC | CAATGA | AGTTTGACAGATGGGGCTGG |
| | | | | T | | |
| HEK24 | GAGCTAAC | caccGAG | aaacCATG | ACACTCTT | TGGAGTTC | TGAATGGATTCCTTGGAAACAATGATAACA |
| | TGTGACAG | CTAACT | CTGTCA | TCCCTACA | AGACGTGT | AGACCTGGCTGAGCTAACTGTGACAGCAT |
| | CATG | GTGAC | CAGTTA | CGACGCTC | GCTCTTCC | GTGGTAATTTTCCAGCCCGCTGGCCCTGTAA |
| | | AGCAT | GCTC | TTCCGATC | GATCTTGA | AGGAAACTGGAACACAAAGCATAGACTGCG |
| | | G | | TNNNNCCA | ATGGATTC | GGGCGGGCCAGCCTGAATAGCTGCAAACAA |
| | | | | GCCCCATC | CTTGGAAA | GTGCAGAATATCTGATGATGTCATACGCAC |
| | | | | TGTCAAAC | CAATGA | AGTTTGACAGATGGGGCTGG |
| | | | | T | | |
| HEK34 | gTGCTTCTC | caccgTG | aaacAGGC | ACACTCTT | TGGAGTTC | ATGTGGGCTGCCTAGAAAGGCATGGATGAG |
| | CAGCCCTG | CTTCTC | CAGGGC | TCCCTACA | AGACGTGT | AGAAGCCTGGAGACAGGGATCCCAGGGAAA |
| | GCCT | CAGCC | TGGAGA | CGACGCTC | GCTCTTCC | CGCCCATGCAATTAGTCTATTTCTGCTGCAA |
| | | CTGGCC | AGCAc | TTCCGATC | GATCTCCC | GTAAGCATGCATTTGTAGGCTTGATGCTTTT |
| | | T | | TNNNNATG | AGCCAAAC | TTTCTGCTTCTCCAGCCCTGGCCTGGGTCA |
| | | | | TGGGCTGC | TTGTCAAC | ATCCTTGGGGCCCAGACTGAGCACGTGATG |
| | | | | CTAGAAAG | C | GCAGAGGAAAGGAAGCCCTGCTTCCTCCAG |
| | | | | G | | AGGGCGTCGCAGGACAGCTTTTCCTAGACA |
| | | | | | | GGGGCTAGTATGTGCAGCTCCTGCACCGGG |
| | | | | | | ATACTGGTTGACAAGTTTGGCTGGG |
| HEK35 | gCGTGCTC | caccgCG | aaacTGGG | ACACTCTT | TGGAGTTC | ATGTGGGCTGCCTAGAAAGGCATGGATGAG |
| | AGTCTGGG | TGCTCA | GCCCAG | TCCCTACA | AGACGTGT | AGAAGCCTGGAGACAGGGATCCCAGGGAAA |
| | CCCCA | GTCTGG | ACTGAG | CGACGCTC | GCTCTTCC | CGCCCATGCAATTAGTCTATTTCTGCTGCAA |
| | | GCCCC | CACGc | TTCCGATC | GATCTCCC | GTAAGCATGCATTTGTAGGCTTGATGCTTTT |
| | | A | | TNNNNATG | AGCCAAAC | TTTCTGCTTCTCCAGCCCTGGCCTGGGTCAA |
| | | | | TGGGCTGC | TTGTCAAC | TCCTTGGGGCCCAGACTGAGCACGTGATG |
| | | | | CTAGAAAG | C | GCAGAGGAAAGGAAGCCCTGCTTCCTCCAG |
| | | | | G | | AGGGCGTCGCAGGACAGCTTTTCCTAGACA |
| | | | | | | GGGGCTAGTATGTGCAGCTCCTGCACCGGG |
| | | | | | | ATACTGGTTGACAAGTTTGGCTGGG |
| HEK37 | gAGCACGT | caccgAG | aaacTTCC | ACACTCTT | TGGAGTTC | ATGTGGGCTGCCTAGAAAGGCATGGATGAG |
| | GATGGCAG | CACGT | TCTGCC | TCCCTACA | AGACGTGT | AGAAGCCTGGAGACAGGGATCCCAGGGAAA |
| | AGGAA | GATGG | ATCACG | CGACGCTC | GCTCTTCC | CGCCCATGCAATTAGTCTATTTCTGCTGCAA |
| | | CAGAG | TGCTc | TTCCGATC | GATCTCCC | GTAAGCATGCATTTGTAGGCTTGATGCTTTT |
| | | GAA | | TNNNNATG | AGCCAAAC | TTTCTGCTTCTCCAGCCCTGGCCTGGGTCAA |
| | | | | TGGGCTGC | TTGTCAAC | TCCTTGGGGCCCAGACTGAGCACGTGATGG |
| | | | | CTAGAAAG | C | CAGAGGAAAGGAAGCCCTGCTTCCTCCAGA |
| | | | | G | | GGGCGTCGCAGGACAGCTTTTCCTAGACAG |
| | | | | | | GGGCTAGTATGTGCAGCTCCTGCACCGGGA |
| | | | | | | TACTGGTTGACAAGTTTGGCTGGG |
| HEK310 | GCACATAC | caccGCA | aaacAGAC | ACACTCTT | TGGAGTTC | ATGTGGGCTGCCTAGAAAGGCATGGATGAG |
| | TAGCCCCT | CATACT | AGGGGC | TCCCTACA | AGACGTGT | AGAAGCCTGGAGACAGGGATCCCAGGGAAA |
| | GTCT | AGCCC | TAGTAT | CGACGCTC | GCTCTTCC | CGCCCATGCAATTAGTCTATTTCTGCTGCAA |
| | | CTGTCT | GTGC | TTCCGATC | GATCTCCC | GTAAGCATGCATTTGTAGGCTTGATGCTTTT |
| | | | | TNNNNATG | AGCCAAAC | TTTCTGCTTCTCCAGCCCTGGCCTGGGTCAA |
| | | | | TGGGCTGC | TTGTCAAC | TCCTTGGGGCCCAGACTGAGCACGTGATGG |
| | | | | CTAGAAAG | C | CAGAGGAAAGGAAGCCCTGCTTCCTCCAGA |
| | | | | G | | GGGCGTCGCAGGACAGCTTTTCCTAGACAG |
| | | | | | | GGGCTAGTATGTGCAGCTCCTGCACCGGG |
| | | | | | | ATACTGGTTGACAAGTTTGGCTGGG |
| HEK411 | gCCCTTCA | caccgCC | aaacTTGT | ACACTCTT | TGGAGTTC | GAACCCAGGTAGCCAGAGACCCGCTGGTCT |
| | AGATGGCT | CTTCAA | CAGCCA | TCCCTACA | AGACGTGT | TCTTTCCCCTCCCCTGCCCTCCCCTCCCTTC |
| | GACAA | GATGG | TCTTGA | CGACGCTC | GCTCTTCC | AAGATGGCTGACAAAGGCCGGGCTGGGTG |
| | | CTGAC | AGGGc | TTCCGATC | GATCTTCC | GAAGGAAGGGAGGAAGGGCGAGGCAGAGG |
| | | AA | | TNNNNGA | TTTCAACC | GTCCAAAGCAGGATGACAGGCAGGGGCACC |
| | | | | ACCCAGGT | CGAACGG | GCGGCGCCCCGGTGGCACTGCGGCTGGAGG |
| | | | | AGCCAGA | AG | TGGGGGTTAAAGCGGAGACTCTGGTGCTGT |
| | | | | GAC | | GTGACTACAGTGGGGGCCCTGCCCTCTCTGA |
| | | | | | | GCCCCCGCCTCCAGGCCTGTGTGTGTGTCTC |
| | | | | | | CGTTCGGGTTGAAAGGA |
| HEK43 | GCCATCTT | caccGCC | aaacTCCC | ACACTCTT | TGGAGTTC | GAACCCAGGTAGCCAGAGACCCGCTGGTCT |
| | GAAGGGAG | ATCTTG | CTCCCTT | TCCCTACA | AGACGTGT | TCTTTCCCCTCCCCTGCCCTCCCCTCCCTTC |
| | GGGA | AAGGG | CAAGAT | CGACGCTC | GCTCTTCC | AAGATGGCTGACAAAGGCCGGGCTGGGTG |
| | | AGGGG | GGC | TTCCGATC | GATCTTCC | GAAGGAAGGGAGGAAGGGCGAGGCAGAGG |
| | | A | | TNNNNGA | TTTCAACC | GTCCAAAGCAGGATGACAGGCAGGGGCACC |
| | | | | ACCCAGGT | CGAACGG | GCGGCGCCCCGGTGGCACTGCGGCTGGAGG |
| | | | | AGCCAGA | AG | TGGGGGTTAAAGCGGAGACTCTGGTGCTGT |
| | | | | GAC | | GTGACTACAGTGGGGGCCCTGCCCTCTCTGA |
| | | | | | | GCCCCCGCCTCCAGGCCTGTGTGTGTGTCTC |
| | | | | | | CGTTCGGGTTGAAAGGA |
| KCNQ2 | gCGGCTCT | caccgCG | aaacCAAA | ACACTCTT | TGGAGTTC | CTCTGTCCAGCACCATGAGCACCGGCAGCA |
| R541T | GATGCTGA | GCTCTG | GTCAGC | TCCCTACA | AGACGTGT | GGCAGGACCACCGAGCGGGAGGCCCCTCCT |
| | CTTTG | ATGCTG | ATCAGA | CGACGCTC | GCTCTTCC | CACTCCCCCAGGCTCCCGGCTGGGCAGGGG |
| | | ACTTTG | GCCGc | TTCCGATC | GATCTNNN | CCTCACCACACGGCTCTGATGCTGACTTT |
| | | | | TNNNNCTC | NGTTTGTG | GAGGCCCGGGGTCAGGTCCTCGGTCACAAA |
| | | | | TGTCCAGC | ACCGAGG | C |
| | | | | ACCATGAG | ACCTGA | |
| | | | | CA | | |
| CTNNB1 | GCTAGGAT | caccGCT | aaacTGAC | ACACTCTT | TGGAGTTC | GGTCCATACCCAAGGCATCCTGGCCATATCC |
| c.2138 | CTAGAAGA | AGGAT | TCTTCTA | TCCCTACA | AGACGTGT | ACCAGAGTGAAAAGAACGATAGCTAGGAT |
| −1 | GTCA | CTAGA | GATCCT | CGACGCTC | GCTCTTCC | CTAGAAGAGTCAGGGTGTCAACAAAATAG |
| G > C | | AGAGT | AGC | TTCCGATC | GATCTNNN | GCAAGAAGGAAGGCAAAAGAGAGAGGAGA |
| | | CA | | TNNNNGGT | NTGGATGC | GAAGCAGACATAGACGTTAACACTGAGGTT |
| | | | | CCATACCC | CCTAACCT | AGGGCATCCA |
| | | | | AAGGCATC | CAGTG | |
| | | | | C | | |
| DIS3L2 | GTGCCATC | caccGTG | aaacTCCC | ACACTCTT | TGGAGTTC | GTGTGTACAGGGGCACATTGAGCGCGTAGT |
| c.2011 | TGCGGGAC | CCATCT | GTCCCG | TCCCTACA | AGACGTGT | GCCGGAACTGCGCTGGGTCCTGCAGCAGCC |
| −1 | GGGA | GCGGG | CAGATG | CGACGCTC | GCTCTTCC | CCGAGCAGAAGTACAGTGCCATCTGCGGG |
| G > C | | ACGGG | GCAC | TTCCGATC | GATCTNNN | ACGGGATGGGTCAGAGCCTGACAAGCCCAG |
| | | A | | TNNNNGTG | NTTAGGTC | AGCTGCCCAGCCAGGCCTGGAAGGCTGGCA |
| | | | | TGTACAGG | TGTCCACA | CCACCCACAGCCTCACCGTCGGCAGCGATG |
| | | | | GGCACATT | CATCGC | TGTGGACAGACCTAA |
| | | | | G | | |
| KCNQ2 | GTCCACTC | caccGTC | aaacCTGT | ACACTCTT | TGGAGTTC | GTCCTCGGGCAGCTCCGCCTCGGCCGGGCCC |
| c.1764 | TACCGGGA | CACTCT | TCCCGG | TCCCTACA | AGACGTGT | TTGGTGCGGTCCTTGTCCGTGATCGCTGGGC |
| −1 | ACAG | ACCGG | TAGAGT | CGACGCTC | GCTCTTCC | CCCGCCCCACGATCTGGTCCACTCTACCGG |
| G > C | | GAACA | GGAC | TTCCGATC | GATCTNNN | GAACAGAGACCCCAAAGCATGAGTTCGGGT |
| (NG) | | G | | TNNNNGTC | NGCCTGGT | GGGTGCAGCAGGGCCCCTGCCCTCTCCTCCT |
| | | | | CTCGGGCA | CCAGGAG | GGACCAGGC |
| | | | | GCTCC | GAG | |

TABLE 7
Off-Targets

| | Forward | Reverse | | | |
|---|---|---|---|---|---|
| | homology to | homology to | | | Genomic amplicon |
| | genome | genome | | | (italics designates the |
| | (5′-3′) | (5′-3′) | Forward primer | Reverse primer | off-target sequence homologous |
| | (SEQ ID NOs: | (SEQ ID NOs: | (SEQ ID NOs: | (SEQ ID NOs: | to the protospacer) |
| Site | 623-638) | 639-652) | 653-666) | 667-680) | (SEQ ID NOs: 681-694) |
| HEK2 | GTGTGGAGA | ACGGTAGGAT | ACACTCTTTC | TGGAGTTCAG | GTGTGGAGAGTGAGTAAGCCAGAACAC |
| OT1 | GTGAGTAAG | GATTTCAGGC | CCTACACGAC | ACGTGTGCTC | AATGCATAGATTGCCGGTAAATAGGTTTA |
| | CCA | A | GCTCTTCCGA | TTCCGATCTA | GATTCATCCATTTTTAAAAAATGGTGTG |
| | | | TCTNNNNGTG | CGGTAGGATG | GGAGCATTAAATATGTATATAGTAGAT |
| | | | TGGAGAGTGA | ATTTCAGGCA | ATGGAAAAATGATTCTCATAATAACTG |
| | | | GTAAGCCA | | ACATTTCTGTTTCACAAGAAAATTATTT |
| | | | | | TACATTATATGTATATTTTACATAAATT |
| | | | | | ATACATAGTCATTTAAAAAGCTCAAAT |
| | | | | | AGTGCAAAAACAATATGGAGAATTGCC |
| | | | | | TGAAATCATCCTACCGT |
| HEK2 | CACAAAGCA | TTTTTGGTAC | ACACTCTTTC | TGGAGTTCAG | CACAAAGCAGTGTAGCTCAGGGAAGGA |
| OT2 | GTGTAGCTC | TCGAGTGTTA | CCTACACGAC | ACGTGTGCTC | GCAGTGAGTTTGGGCACTTGTGACAGA |
| | AGG | TTCAG | GCTCTTCCGA | TTCCGATCTT | ATAGTGGGACTATGCCAGAGATACACA |
| | | | TCTNNNNCAC | TTTTGGTACT | GGAGGAGGTGGTACCTTCTAGCTCCCC |
| | | | AAAGCAGTGT | CGAGTGTTAT | CTCAAAACATAAAGCATAGACTGCAAAGT |
| | | | AGCTCAGG | TCAG | ACTCCCAAGCAGGCTGAATAACACTCG |
| | | | | | AGTACCAAAAA |
| HEK3 | TCCCCTGTTG | CACTGTACTT | ACACTCTTTC | TGGAGTTCAG | TCCCCTGTTGACCTGGAGAAGCATGAA |
| OT1 | ACCTGGAGA | GCCCTGACCA | CCTACACGAC | ACGTGTGCTC | CCAGTCAAAAAGTTTAAAGACAAGAGC |
| | A | | GCTCTTCCGA | TTCCGATCTC | ATTAACTGCACCAGTGGGCAGCTCAGC |
| | | | TCTNNNNTCC | ACTGTACTTG | TCAGACACCAGTAGCGTGGGCACCCAG |
| | | | CCTGTTGACC | CCCTGACCA | ACTGAGCACGTGCTGGAGCCCAAGAAAT |
| | | | TGGAGAA | | GCAGAGACCTGTGCACCTCTGGTCAGG |
| | | | | | GCAAGTACAGTG |
| HEK3 | TTGGTGTTG | CTGAGATGTG | ACACTCTTTC | TGGAGTTCAG | TTGGTGTTGACAGGGAGCAACTTCACA |
| OT2 | ACAGGGAGC | GGCAGAAGG | CCTACACGAC | ACGTGTGCTC | GTCCCAGGCATCAGGACACAGACTGGGC |
| | AA | G | GCTCTTCCGA | TTCCGATCTC | ACGTGAGGGAAGCCCAAGGGAGAGGAC |
| | | | TCTNNNNTTG | TGAGATGTGG | TGGTGTAATCGAGGCTGACTCCACTTTT |
| | | | GTGTTGACAG | GCAGAAGGG | AATGTTTGACTGATGATAGGTTTCAAGT |
| | | | GGAGCAA | | CTCACTAAGTCTCCTTCCCCTTCTGCCC |
| | | | | | ACATCTCAG |
| HEK3 | TGAGAGGGA | GTCCAAAGGC | ACACTCTTTC | TGGAGTTCAG | TGAGAGGGAACAGAAGGGCTAAGACTA |
| OT3 | ACAGAAGGG | CCAAGAACCT | CCTACACGAC | ACGTGTGCTC | AAAGGAACAGAGGAGTTCATAGTGAGC |
| | CT | | GCTCTTCCGA | TTCCGATCTG | GGTAAAGAGCTCAGACTGAGCAAGTGAG |
| | | | TCTNNNNTGA | TCCAAAGGCC | GGGCTCAGCCTCCCATGGAGGACAGGG |
| | | | GAGGGAACA | CAAGAACCT | GGCTGGGGCCCCTGGCTGATGTCTGGA |
| | | | GAAGGGCT | | CTGAAGCCCCCACGCCCAGAGGTTCTT |
| | | | | | GGGCCTTTGGAC |
| HEK3 | TCCTAGCAC | GCTCATCTTA | ACACTCTTTC | TGGAGTTCAG | TCCTAGCACTTTGGAAGGTCGAAGCGG |
| OT4 | TTTGGAAGG | ATCTGCTCAG | CCTACACGAC | ACGTGTGCTC | CAGGATGGCTTCAACCCAGGAGTTCGA |
| | TCG | CC | GCTCTTCCGA | TTCCGATCTG | GACCAGACTGAGCAAGAGAGGGAGAGTG |
| | | | TCTNNNNTCC | CTCATCTTAA | TCTGTATTAACAACAAACAAACAAACA |
| | | | TAGCACTTTG | TCTGCTCAGC | AAAAACTAAACTAAAAGAAACTGTGGT |
| | | | GAAGGTCG | C | GTATAATATAAAATTCTGGCTGAGCAG |
| | | | | | ATTAAGATGAGC |
| HEK4 | GGCATGGCT | TGTCCCCTTG | ACACTCTTTC | TGGAGTTCAG | GGCATGGCTTCTGAGACTCATAGCTGG |
| OT1 | TCTGAGACT | CACTCCCTGT | CCTACACGAC | ACGTGTGCTC | GGCTGAAGATCCCTAGGGGGGCTCTGC |
| | CA | CTTT | GCTCTTCCGA | TTCCGATCTT | TGGGCTCACTGCTCTCCAGAGTGGTCCA |
| | | | TCTNNNNGGC | GTCCCCTTGC | GCCCGGCTGCAGGGTGCTGCTTCCAGCT |
| | | | ATGGCTTCTG | ACTCCCTGTC | TGGTGCACTGCGGCCGGAGGAGGTGGA |
| | | | AGACTCA | TTT | GGATGGAAAGTAAGATTCAAAGACAGG |
| | | | | | GAGTGCAAGGG |
| HEK4 | GAAGAGGCT | TTTGGCAATG | ACACTCTTTC | TGGAGTTCAG | GAAGAGGCTGCCCATGAGAGCAAGGGA |
| OT2 | GCCCATGAG | GAGGCATTGG | CCTACACGAC | ACGTGTGCTC | GCCGAAGCAAGTGCTCCCCAATCCTGA |
| | AG | | GCTCTTCCGA | TTCCGATCTT | AACCTGCCTGGCTGGGGCCCCTGTCACT |
| | | | TCTNNNNGAA | TTGGCAATGG | AACAGCAACCCCACCCCCTCTAGCCGCA |
| | | | GAGGCTGCCC | AGGCATTGG | GAGCCCTGCGCACGTGCATGTGCCCTGA |
| | | | ATGAGAG | | AGACAGGCTTCCCCTGCCCAATGCCTCC |
| | | | | | ATTGCCAAA |
| HEK4 | GGTCTGAGG | CTGTGGCCTC | ACACTCTTTC | TGGAGTTCAG | GGTCTGAGGCTCGAATCCTGGCAGCAG |
| OT3 | CTCGAATCC | CATATCCCTG | CCTACACGAC | ACGTGTGCTC | GTCCTTCATGGCAAGGCGGGAAAAGAG |
| | TG | | GCTCTTCCGA | TTCCGATCTC | AAAAGCCAACGGGTTCTCATGCTGGGA |
| | | | TCTNNNNGGT | TGTGGCCTCC | AAAGATGCCGGGCACGACGGCTGGAGG |
| | | | CTGAGGCTCG | ATATCCCTG | TGGGGGGTTGGGAGTGGGTGGGATGCT |
| | | | AATCCTG | | TGCGTGCCCTGCATGAGGTGCAGGGAT |
| | | | | | ATGGAGGCCACAG |
| HEK4 | TTTCCACCA | CCTCGGTTCC | ACACTCTTTC | TGGAGTTCAG | TTTCCACCAGAACTCAGCCCAGGCTGCT |
| OT4 | GAACTCAGC | TCCACAACAC | CCTACACGAC | ACGTGTGCTC | GTGGGATGGAATCACCTGCACCCGGAT |
| | CC | | GCTCTTCCGA | TTCCGATCTC | GTTCTTTCTGGGCTGGTACATACAGGCA |
| | | | TCTNNNNTTT | CTCGGTTCCT | AGGCATCACGGCTGGAGGTGGAGGGGG |
| | | | CCACCAGAAC | CCACAACAC | CCTAACCCGGGGTTGCCCAGGAAGGGG |
| | | | TCAGCCC | | TTTGCACATGGATTCGGTGTGTTGTGGA |
| | | | | | GGAACCGAGG |
| FANCF | GCGGGCAGT | CCCTGGGTTT | ACACTCTTTC | TGGAGTTCAG | GCGGGCAGTGGCGTCTTAGTCGCCTTA |
| OT1 | GGCGTCTTA | GGTTGGCTGC | CCTACACGAC | ACGTGTGCTC | GCACTGGGTGCTTAATCCGGCTCCATCT |
| | GTCG | TC | GCTCTTCCGA | TTCCGATCTC | TTTCTCCACGGAGGGGGCCTGGTGCTGC |
| | | | TCTNNNNGCG | CCTGGGTTTG | AGACGGGGTTCCCGGGGTCAGGACGAT |
| | | | GGCAGTGGCG | GTTGGCTGCT | CCAGGTGACTTGAGAGAAAATAAGGGG |
| | | | TCTTAGTCG | C | AGTTGTATTGACACCAACTGTTTTATTT |
| | | | | | ATTGTGATCTTCAGGTTAGTAAACAACT |
| | | | | | CCAGTGGCATCAATCTGTGTATCTGTTA |
| | | | | | AGTCTTAATGAGCAGCCAACCAAACCC |
| | | | | | AGGG |
| FANCF | CTCCTTGCCG | CACTGGGGAA | ACACTCTTTC | TGGAGTTCAG | CTCCTTGCCGCCCAGCCGGTCCAGGCCT |
| OT2 | CCCAGCCGG | GAGGCGAGG | CCTACACGAC | ACGTGTGCTC | CTGGCGAACATGGCGCTTGTCCCCTGCC |
| | TC | ACAC | GCTCTTCCGA | TTCCGATCTC | AGGTGCTGCGGATGGCAATCCTGCTGTC |
| | | | TCTNNNNCTC | ACTGGGGAA | TTACTGCTCTATCCTGTGTAACTACAAG |
| | | | CTTGCCGCCC | GAGGCGAGG | GCCATCGAAATGCCCTCACACCAGACC |
| | | | AGCCGGTC | ACAC | TACGGAGGGAGCTGGAAATTCCTGACG |
| | | | | | TTCATTGATCTGGTAAGGCCGTCCCCTC |
| | | | | | CCCCTGCTCGCCCCGCACCCCGTGCCTG |
| | | | | | TGTGTGCGTGTGTGTGTGTGTGTGTGTG |
| | | | | | TGTGTGTGTGGATGCGCGAGCACCTGC |
| | | | | | GCACGTGCGCGCCTCCAGCATCCACCC |
| | | | | | GTGTCCTCGCCTCTTCCCCAGTG |
| FANCF | CCAGTGTTTC | GAATGGATCC | ACACTCTTTC | TGGAGTTCAG | CCAGTGTTTCCCATCCCCAACACAGTGA |
| OT3 | CCATCCCCA | CCCCCTAGAG | CCTACACGAC | ACGTGTGCTC | CAGAAGGCAGCCAAGGAATCCTCATTC |
| | ACAC | CTC | GCTCTTCCGA | TTCCGATCTG | CTGTCCTGGAACTACAGGAGTCCCTCCT |
| | | | TCTNNNNCCA | AATGGATCCC | ACAGCACCAGGTGTATTCATCTTCTGTT |
| | | | GTGTTTCCCA | CCCCTAGAGC | GTTGCTATAACAAAATTACCACAAACTT |
| | | | TCCCCAACAC | TC | AGTGGCTTAAGTAACTACACATTTATTA |
| | | | | | TTTTCCAGTTGTGGAGGTCAGAGGTCTC |
| | | | | | AAACTGGTCTCACTGGGAAAAACTCAA |
| | | | | | GGTCTTCAGGGCTGTATTCCCTTTGGAG |
| | | | | | CTCTAGGGGGGGATCCATTC |
| FANCF | CAGGCCCAC | CCACACGGAA | ACACTCTTTC | TGGAGTTCAG | CAGGCCCACAGGTCCTTCTGGAAGGAC |
| OT4 | AGGTCCTTCT | GGCTGACCAC | CCTACACGAC | ACGTGTGCTC | TCAGGCAGGAGTTAGGAGGCTCCCGGG |
| | GGA | G | GCTCTTCCGA | TTCCGATCTC | GTCAGGCTTCTGGGTCTAGATTTCCAGA |
| | | | TCTNNNNCAG | CACACGGAA | GGCCCCTCTGCAGCACCAGGCATTCGCC |
| | | | GCCCACAGGT | GGCTGACCAC | TCTAGGAGTCATCGCTCTTCAGCGGATC |
| | | | CCTTCTGGA | G | CTGCAGCCCTTGGCGATGCTCAGAGTG |
| | | | | | AACGCGTTACCCCGCCAGCCCCCCTCTG |
| | | | | | CCGGCTCCTGCCGGTTTGTGATTTCTGT |
| | | | | | GTCTTCGTCTGTGGCCTGTGGATGTGGC |
| | | | | | CTTACACCTCGTGGTCAGCCTTCCGTGT |
| | | | | | GG |

TABLE 8
Sequences of the Domains of Exemplary CGBE Fusion Proteins

| | | |
|---|---|---|
| | | SEQ |
| | | ID |
| Name | Sequence | NO: |

Deaminase domains

| | | |
|---|---|---|
| rAPOBEC | MSSETGPVAVDPTLRRRIEPHEFEVFFDPRELRKETCLLYEINWGGRHSIWRHTS | 695 |
| | QNTNKHVEVNFIEKFTTERYFCPNTRCSITWFLSWSPCGECSRAITEFLSRYPHV | |
| | TLFIYIARLYHHADPRNRQGLRDLISSGVTIQIMTEQESGYCWRNFVNYSPSNEA | |
| | HWPRYPHLWVRLYVLELYCIILGLPPCLNILRRKQPQLTFFTIALQSCHYQRLPP | |
| | HILWATGLK | |
| EE (rAPOBEC1 | MSSETGPVAVDPTLRRRIEPHEFEVFFDPRELRKETCLLYEINWGGRHSIWRHTS | 696 |
| R126E, R132E) | QNTNKHVEVNFIEKFTTERYFCPNTRCSITWFLSWSPCGECSRAITEFLSRYPHV | |
| | TLFIYIARLYHHADPENRQGLEDLISSGVTIQIMTEQESGYCWRNFVNYSPSNEA | |
| | HWPRYPHLWVRLYVLELYCIILGLPPCLNILRRKQPQLTFFTIALQSCHYQRLPP | |
| | HILWATGLK | |
| YE1 (rAPOBEC1 | MSSETGPVAVDPTLRRRIEPHEFEVFFDPRELRKETCLLYEINWGGRHSIWRHTS | 697 |
| W90Y, R126E) | QNTNKHVEVNFIEKFTTERYFCPNTRCSITWFLSYSPCGECSRAITEFLSRYPHVT | |
| | LFIYIARLYHHADPENRQGLRDLISSGVTIQIMTEQESGYCWRNFVNYSPSNEAH | |
| | WPRYPHLWVRLYVLELYCIILGLPPCLNILRRKQPQLTFFTIALQSCHYQRLPPHI | |
| | LWATGLK | |
| YE2 (rAPOBEC1 | MSSETGPVAVDPTLRRRIEPHEFEVFFDPRELRKETCLLYEINWGGRHSIWRHTS | 698 |
| W90Y, R132E) | QNTNKHVEVNFIEKFTTERYFCPNTRCSITWFLSYSPCGECSRAITEFLSRYPHVT | |
| | LFIYIARLYHHADPRNRQGLEDLISSGVTIQIMTEQESGYCWRNFVNYSPSNEAH | |
| | WPRYPHLWVRLYVLELYCIILGLPPCLNILRRKQPQLTFFTIALQSCHYQRLPPHI | |
| | LWATGLK | |
| YEE (rAPOBEC1 | MSSETGPVAVDPTLRRRIEPHEFEVFFDPRELRKETCLLYEINWGGRHSIWRHTS | 699 |
| W90Y, R126E, R132E) | QNTNKHVEVNFIEKFTTERYFCPNTRCSITWFLSYSPCGECSRAITEFLSRYPHVT | |
| | LFIYIARLYHHADPENRQGLEDLISSGVTIQIMTEQESGYCWRNFVNYSPSNEAH | |
| | WPRYPHLWVRLYVLELYCIILGLPPCLNILRRKQPQLTFFTIALQSCHYQRLPPHI | |
| | LWATGLK | |
| Anc68919 | MSSETGPVAVDPTLRRRIEPHEFEVFFDPRELRKETCLLYEIKWGTSHKIWRHSS | 700 |
| | KNTTKHVEVNFIEKFTSERHFCPSTSCSITWFLSWSPCGECSKAITEFLSQHPNVT | |
| | LVIYVARLYHHMDQQNRQGLRDLVNSGVTIQIMTAPEYDYCWRNFVNYPPGK | |
| | EAHWPRYPPLWMKLYALELHAGILGLPPCLNILRRKQPQLTFFTIALQSCHYQR | |
| | LPPHI | |
| eA3A 30 | MEASPASGPRHLMDPHIFTSNFNNGIGRHKTYLCYEVERLDNGTSVKMDQHRG | 701 |
| | FLHGQAKNLLCGFYGRHAELRFLDLVPSLQLDPAQIYRVTWFISWSPCFSWGCA | |
| | GEVRAFLQENTHVRLRIFAARIYDYDPLYKEALQMLRDAGAQVSIMTYDEFKH | |
| | CWDTFVDHQGCPFQPWDGLDEHSQALSGRLRAILQNQGN | |
| eA3A* (T31A) | MEASPASGPRHLMDPHIFTSNFNNGIGRHKAYLCYEVERLDNGTSVKMDQHRG | 702 |
| | FLHGQAKNLLCGFYGRHAELRFLDLVPSLQLDPAQIYRVTWFISWSPCFSWGCA | |
| | GEVRAFLQENTHVRLRIFAARIYDYDPLYKEALQMLRDAGAQVSIMTYDEFKH | |
| | CWDTFVDHQGCPFQPWDGLDEHSQALSGRLRAILQNQGN | |

Glycosylase fusion domains

| | | |
|---|---|---|
| UdgX | MAGAQDFVPHTADLAELAAAAGECRGCGLYRDATQAVFGAGGRSARIMMIGE | 703 |
| | QPGDKEDLAGLPFVGPAGRLLDRALEAADIDRDALYVTNAVKHFKFTRAAGG | |
| | KRRIHKTPSRTEVVACRPWLIAEMTSVEPDVVVLLGATAAKALLGNDFRVTQH | |
| | RGEVLHVDDVPGDPALVATVHPSSLLRGPKEERESAFAGLVDDLRVAADVRP | |
| UNG2 | MIGQKTLYSFFSPSPARKRHAPSPEPAVQGTGVAGVPEESGDAAAIPAKKAPAG | 704 |
| | QEEPGTPPSSPLSAEQLDRIQRNKAAALLRLAARNVPVGFGESWKKHLSGEFGK | |
| | PYFIKLMGFVAEERKHYTVYPPPHQVFTWTQMCDIKDVKVVILGQDPYHGPNQ | |
| | AHGLCFSVQRPVPPPPSLENIYKELSTDIEDFVHPGHGDLSGWAKQGVLLLNAV | |
| | LTVRAHQANSHKERGWEQFTDAVVSWLNQNSNGLVFLLWGSYAQKKGSAIDR | |
| | KRHHVLQTAHPSPLSVYRGFFGCRHFSKTNELLQKSGKKPIDWKEL | |
| SMUG1 | MPQAFLLGSIHEPAGALMEPQPCPGSLAESFLEEELRLNAELSQLQFSEPVGIIYN | 705 |
| | PVEYAWEPHRNYVTRYCQGPKEVLFLGMNPGPFGMAQTGVPFGEVSMVRDW | |
| | LGIVGPVLTPPQEHPKRPVLGLECPQSEVSGARFWGFFRNLCGQPEVFFHHCFV | |
| | HNLCPLLFLAPSGRNLTPAELPAKQREQLLGICDAALCRQVQLLGVRLVVGVGR | |
| | LAEQRARRALAGLMPEVQVEGLLHPSPRNPQANKGWEAVAKERLNELGLLPLL | |
| | LK | |
| MBD4 | MGTTGLESLSLGDRGAAPTVTSSERLVPDPPNDLRKEDVAMELERVGEDEEQM | 706 |
| | MIKRSSECNPLLQEPIASAQFGATAGTECRKSVPCGWERVVKQRLFGKTAGRFD | |
| | VYFISPQGLKFRSKSSLANYLHKNGETSLKPEDFDFTVLSKRGIKSRYKDCSMA | |
| | ALTSHLQNQSNNSNWNLRTRSKCKKDVFMPPSSSSELQESRGLSNFTSTHLLLK | |
| | EDEGVDDVNFRKVRKPKGKVTILKGIPIKKTKKGCRKSCSGFVQSDSKRESVCN | |
| | KADAESEPVAQKSQLDRTVCISDAGACGETLSVTSEENSLVKKKERSLSSGSNF | |
| | CSEQKTSGIINKFCSAKDSEHNEKYEDTFLESEEIGTKVEVVERKEHLHTDILKR | |
| | GSEMDNNCSPTRKDFTGEKIFQEDTIPRTQIERRKTSLYFSSKYNKEALSPPRRK | |
| | AFKKWTPPRSPFNLVQETLFHDPWKLLIATIFLNRTSGKMAIPVLWKFLEKYPSA | |
| | EVARTADWRDVSELLKPLGLYDLRAKTIVKFSDEYLTKQWKYPIELHGIGKYG | |
| | NDSYRIFCVNEWKQVHPEDHKLNKYHDWLWENHEKLSL | |
| TDG | MEAENAGSYSLQQAQAFYTFPFQQLMAEAPNMAVVNEQQMPEEVPAPAPAQE | 707 |
| | PVQEAPKGRKRKPRTTEPKQPVEPKKPVESKKSGKSAKSKEKQEKITDTFKVKR | |
| | KVDRFNGVSEAELLTKTLPDILTFNLDIVIIGINPGLMAAYKGHHYPGPGNHFW | |
| | KCLFMSGLSEVQLNHMDDHTLPGKYGIGFTNMVERTTPGSKDLSSKEFREGGRI | |
| | LVQKLQKYQPRIAVFNGKCIYEIFSKEVFGVKVKNLEFGLQPHKIPDTETLCYV | |
| | MPSSSARCAQFPRAQDKVHYYIKLKDLRDQLKGIERNMDVQEVQYTFDLQLAQ | |
| | EDAKKMAVKEEKYDPGYEAAYGGAYGENPCSSEPCGFSSNGLIESVELRGESA | |
| | FSGIPNGQWMTQSFTDQIPSFSNHCGTQEQEEESHA | |

CRISPRi screen hit fusion domains

| | | |
|---|---|---|
| DDX1 | MAAFSEMGVMPEIAQAVEEMDWLLPTDIQAESIPLILGGGDVLMAAETGSGKT | 708 |
| | GAFSIPVIQIVYETLKDQQEGKKGKTTIKTGASVLNKWQMNPYDRGSAFAIGSD | |
| | GLCCQSREVKEWHGCRATKGLMKGKHYYEVSCHDQGLCRVGWSTMQASLDL | |
| | GTDKFGFGFGGTGKKSHNKQFDNYGEEFTMHDTIGCYLDIDKGHVKFSKNGKD | |
| | LGLAFEIPPHMKNQALFPACVLKNAELKFNFGEEEFKFPPKDGFVALSKAPDGYI | |
| | VKSQHSGNAQVTQTKFLPNAPKALIVEPSRELAEQTLNNIKQFKKYIDNPKLREL | |
| | LIIGGVAARDQLSVLENGVDIVVGTPGRLDDLVSTGKLNLSQVRFLVLDEADGL | |
| | LSQGYSDFINRMHNQIPQVTSDGKRLQVIVCSATLHSFDVKKLSEKIMHFPTWV | |
| | DLKGEDSVPDTVHHVVVPVNPKTDRLWERLGKSHIRTDDVHAKDNTRPGANS | |
| | PEMWSEAIKILKGEYAVRAIKEHKMDQAIIFCRTKIDCDNLEQYFIQQGGGPDK | |
| | KGHQFSCVCLHGDRKPHERKQNLERFKKGDVRFLICTDVAARGIDIHGVPYVIN | |
| | VTLPDEKQNYVHRIGRVGRAERMGLAISLVATEKEKVWYHVCSSRGKGCYNT | |
| | RLKEDGGCTIWYNEMQLLSEIEEHLNCTISQVEPDIKVPVDEFDGKVTYGQKRA | |
| | AGGGSYKGHVDILAPTVQELAALEKEAQTSFLHLGYLPNQLFRTF | |
| EXO1 | MGIQGLLQFIKEASEPIHVRKYKGQVVAVDTYCWLHKGAIACAEKLAKGEPTD | 709 |
| | RYVGFCMKFVNMLLSHGIKPILVFDGCTLPSKKEVERSRRERRQANLLKGKQLL | |
| | REGKVSEARECFTRSINITHAMAHKVIKAARSQGVDCLVAPYEADAQLAYLNK | |
| | AGIVQAIITEDSDLLAFGCKKVILKMDQFGNGLEIDQARLGMCRQLGDVFTEEK | |
| | FRYMCILSGCDYLSSLRGIGLAKACKVLRLANNPDIVKVIKKIGHYLKMNITVPE | |
| | DYINGFIRANNTFLYQLVFDPIKRKLIPLNAYEDDVDPETLSYAGQYVDDSIALQ | |
| | IALGNKDINTFEQIDDYNPDTAMPAHSRSHSWDDKTCQKSANVSSIWHRNYSPR | |
| | PESGTVSDAPQLKENPSTVGVERVISTKGLNLPRKSSIVKRPRSAELSEDDLLSQ | |
| | YSLSFTKKTKKNSSEGNKSLSFSEVFVPDLVNGPTNKKSVSTPPRTRNKFATFLQ | |
| | RKNEESGAVVVPGTRSRFFCSSDSTDCVSNKVSIQPLDETAVTDKENNLHESEY | |
| | GDQEGKRLVDTDVARNSSDDIPNNHIPGDHIPDKATVFTDEESYSFESSKFTRTIS | |
| | PPTLGTLRSCFSWSGGLGDFSRTPSPSPSTALQQFRRKSDSPTSLPENNMSDVSQ | |
| | LKSEESSDDESHPLREEACSSQSQESGEFSLQSSNASKLSQCSSKDSDSEESDCNI | |
| | KLLDSQSDQTSKLRLSHFSKKDTPLRNKVPGLYKSSSADSLSTTKIKPLGPARAS | |
| | GLSKKPASIQKRKHHNAENKPGLQIKLNELWKNFGFKKDSEKLPPCKKPLSPVR | |
| | DNIQLTPEAEEDIFNKPECGRVQRAIFQ | |
| PCNA | MFEARLVQGSILKKVLEALKDLINEACWDISSSGVNLQSMDSSHVSLVQLTLRS | 710 |
| | EGFDTYRCDRNLAMGVNLTSMSKILKCAGNEDIITLRAEDNADTLALVFEAPNQ | |
| | EKVSDYEMKLMDLDVEQLGIPEQEYSCVVKMPSGEFARICRDLSHIGDAVVISC | |
| | AKDGVKFSASGELGNGNIKLSQTSNVDKEEEAVTIEMNEPVQLTFALRYLNFFT | |
| | KATPLSSTVTLSMSADVPLVVEYKIADMGHLKYYLAPKIEDEEGS | |
| POLD1 | MDGKRRPGPGPGVPPKRARGGLWDDDDAPRPSQFEEDLALMEEMEAEHRLQE | 711 |
| | QEEEELQSVLEGVADGQVPPSAIDPRWLRPTPPALDPQTEPLIFQQLEIDHYVGP | |
| | AQPVPGGPPPSRGSVPVLRAFGVTDEGFSVCCHIHGFAPYFYTPAPPGFGPEHM | |
| | GDLQRELNLAISRDSRGGRELTGPAVLAVELCSRESMFGYHGHGPSPFLRITVAL | |
| | PRLVAPARRLLEQGIRVAGLGTPSFAPYEANVDFEIRFMVDTDIVGCNWLELPA | |
| | GKYALRLKEKATQCQLEADVLWSDVVSHPPEGPWQRIAPLRVLSFDIECAGRK | |
| | GIFPEPERDPVIQICSLGLRWGEPEPFLRLALTLRPCAPILGAKVQSYEKEEDLLQ | |
| | AWSTFIRIMDPDVITGYNIQNFDLPYLISRAQTLKVQTFPFLGRVAGLCSNIRDSS | |
| | FQSKQTGRRDTKVVSMVGRVQMDMLQVLLREYKLRSYTLNAVSFHFLGEQKE | |
| | DVQHSIITDLQNGNDQTRRRLAVYCLKDAYLPLRLLERLMVLVNAVEMARVT | |
| | GVPLSYLLSRGQQVKVVSQLLRQAMHEGLLMPVVKSEGGEDYTGATVIEPLKG | |
| | YYDVPIATLDFSSLYPSIMMAHNLCYTTLLRPGTAQKLGLTEDQFIRTPTGDEFV | |
| | KTSVRKGLLPQILENLLSARKRAKAELAKETDPLRRQVLDGRQLALKVSANSV | |
| | YGFTGAQVGKLPCLEISQSVTGFGRQMIEKTKQLVESKYTVENGYSTSAKVVY | |
| | GDTDSVMCRFGVSSVAEAMALGREAADWVSGHFPSPIRLEFEKVYFPYLLISKK | |
| | RYAGLLFSSRPDAHDRMDCKGLEAVRRDNCPLVANLVTASLRRLLIDRDPEGA | |
| | VAHAQDVISDLLCNRIDISQLVITKELTRAASDYAGKQAHVELAERMRKRDPGS | |
| | APSLGDRVPYVIISAAKGVAAYMKSEDPLFVLEHSLPIDTQYYLEQQLAKPLLRI | |
| | FEPILGEGRAEAVLLRGDHTRCKTVLTGKVGGLLAFAKRRNCCIGCRTVLSHQG | |
| | AVCEFCQPRESELYQKEVSHLNALEERFSRLWTQCQRCQGSLHEDVICTSRDCPI | |
| | FYMRKKVRKDLEDQEQLLRRFGPPGPEAW | |
| POLD2 | MFSEQAAQRAHTLLSPPSANNATFARVPVATYTNSSQPFRLGERSFSRQYAHIY | 712 |
| | ATRLIQMRPFLENRAQQHWGSGVGVKKLCELQPEEKCCVVGTLFKAMPLQPSI | |
| | LREVSEEHNLLPQPPRSKYIHPDDELVLEDELQRIKLKGTIDVSKLVTGTVLAVF | |
| | GSVRDDGKFLVEDYCFADLAPQKPAPPLDTDRFVLLVSGLGLGGGGGESLLGT | |
| | QLLVDVVTGQLGDEGEQCSAAHVSRVILAGNLLSHSTQSRDSINKAKYLTKKT | |
| | QAASVEAVKMLDEILLQLSASVPVDVMPGEFDPTNYTLPQQPLHPCMFPLATA | |
| | YSTLQLVTNPYQATIDGVRFLGTSGQNVSDIFRYSSMEDHLEILEWTLRVRHISP | |
| | TAPDTLGCYPFYKTDPFIFPECPHVYFCGNTPSFGSKIIRGPEDQTVLLVTVPDFS | |
| | ATQTACLVNLRSLACQPISFSGFGAEDDDLGGLGLGP | |
| POLD3 | MADQLYLENIDEFVTDQNKIVTYKWLSYTLGVHVNQAKQMLYDYVERKRKE | 713 |
| | NSGAQLHVTYLVSGSLIQNGHSCHKVAVVREDKLEAVKSKLAVTASIHVYSIQ | |
| | KAMLKDSGPLFNTDYDILKSNLQNCSKFSAIQCAAAVPRAPAESSSSSKKFEQSH | |
| | LHMSSETQANNELTTNGHGPPASKQVSQQPKGIMGMFASKAAAKTQETNKET | |
| | KTEAKEVTNASAAGNKAPGKGNMMSNFFGKAAMNKFKVNLDSEQAVKEEKI | |
| | VEQPTVSVTEPKLATPAGLKKSSKKAEPVKVLQKEKKRGKRVALSDDETKETE | |
| | NMRKKRRRIKLPESDSSEDEVFPDSPGAYEAESPSPPPPPSPPLEPVPKTEPEPPSV | |
| | KSSSGENKRKRKRVLKSKTYLDGEGCIVTEKVYESESCTDSEEELNMKTSSVHR | |
| | PPAMTVKKEPREERKGPKKGTAALGKANRQVSITGFFQRK | |
| POLH | MATGQDRVVALVDMDCFFVQVEQRQNPHLRNKPCAVVQYKSWKGGGIIAVS | 714 |
| | YEARAFGVTRSMWADDAKKLCPDLLLAQVRESRGKANLTKYREASVEVMEIM | |
| | SRFAVIERASIDEAYVDLTSAVQERLQKLQGQPISADLLPSTYIEGLPQGPTTAEE | |
| | TVQKEGMRKQGLFQWLDSLQIDNLTSPDLQLTVGAVIVEEMRAAIERETGFQCS | |
| | AGISHNKVLAKLACGLNKPNRQTLVSHGSVPQLFSQMPIRKIRSLGGKLGASVIE | |
| | ILGIEYMGELTQFTESQLQSHFGEKNGSWLYAMCRGIEHDPVKPRQLPKTIGCS | |
| | KNFPGKTALATREQVQWWLLQLAQELEERLTKDRNDNDRVATQLVVSIRVQG | |
| | DKRLSSLRRCCALTRYDAHKMSHDAFTVIKNCNTSGIQTEWSPPLTMLFLCATK | |
| | FSASAPSSSTDITSFLSSDPSSLPKVPVTSSEAKTQGSGPAVTATKKATTSLESFFQ | |
| | KAAERQKVKEASLSSLTAPTQAPMSNSPSKPSLPFQTSQSTGTEPFFKQKSLLLK | |
| | QKQLNNSSVSSPQQNPWSNCKALPNSLPTEYPGCVPVCEGVSKLEESSKATPAE | |
| | MDLAHNSQSMHASSASKSVLEVTQKATPNPSLLAAEDQVPCEKCGSLVPVWD | |
| | MPEHMDYHFALELQKSFLQPHSSNPQVVSAVSHQGKRNPKSPLACTNKRPRPE | |
| | GMQTLESFFKPLTH | |
| POLK | MDSTKEKCDSYKDDLLLRMGLNDNKAGMEGLDKEKINKIIMEATKGSRFYGN | 715 |
| | ELKKEKQVNQRIENMMQQKAQITSQQLRKAQLQVDRFAMELEQSRNLSNTIVH | |
| | IDMDAFYAAVEMRDNPELKDKPIAVGSMSMLSTSNYHARRFGVRAAMPGFIAK | |
| | RLCPQLIIVPPNFDKYRAVSKEVKEILADYDPNFMAMSLDEAYLNITKHLEERQ | |
| | NWPEDKRRYFIKMGSSVENDNPGKEVNKLSEHERSISPLLFEESPSDVQPPGDPF | |
| | QVNFEEQNNPQILQNSVVFGTSAQEVVKEIRFRIEQKTTLTASAGIAPNTMLAKV | |
| | CSDKNKPNGQYQILPNRQAVMDFIKDLPIRKVSGIGKVTEKMLKALGIITCTELY | |
| | QQRALLSLLFSETSWHYFLHISLGLGSTHLTRDGERKSMSVERTFSEINKAEEQY | |
| | SLCQELCSELAQDLQKERLKGRTVTIKLKNVNFEVKTRASTVSSVVSTAEEIFAI | |
| | AKELLKTEIDADFPHPLRLRLMGVRISSFPNEEDRKHQQRSIIGFLQAGNQALSA | |
| | TECTLEKTDKDKFVKPLEMSHKKSFFDKKRSERKWSHQDTFKCEAVNKQSFQT | |
| | SQPFQVLKKKMNENLEISENSDDCQILTCPVCFRAQGCISLEALNKHVDECLDG | |
| | PSISENFKMFSCSHVSATKVNKKENVPASSLCEKQDYEAHPKIKEISSVDCIALV | |
| | DTIDNSSKAESIDALSNKHSKEECSSLPSKSFNIEHCHQNSSSTVSLENEDVGSFR | |
| | QEYRQPYLCEVKTGQALVCPVCNVEQKTSDLTLFNVHVDVCLNKSFIQELRKD | |
| | KFNPVNQPKESSRSTGSSSGVQKAVTRTKRPGLMTKYSTSKKIKPNNPKHTLDIF | |
| | FK | |
| RAD18 | MDSLAESRWPPGLAVMKTIDDLLRCGICFEYFNIAMIIPQCSHNYCSLCIRKELS | 716 |
| | YKTQCPTCCVTVTEPDLKNNRILDELVKSLNFARNHLLQFALESPAKSPASSSSK | |
| | NLAVKVYTPVASRQSLKQGSRLMDNFLIREMSGSTSELLIKENKSKFSPQKEASP | |
| | AAKTKETRSVEEIAPDPSEAKRPEPPSTSTLKQVTKVDCPVCGVNIPESHINKHL | |
| | DSCLSREEKKESLRSSVHKRKPLPKTVYNLLSDRDLKKKLKEHGLSIQGNKQQL | |
| | IKRHQEFVHMYNAQCDALHPKSAAEIVREIENIEKTRMRLEASKLNESVMVFTK | |
| | DQTEKEIDEIHSKYRKKHKSEFQLLVDQARKGYKKIAGMSQKTVTITKEDESTE | |
| | KLSSVCMGQEDNMTSVTNHFSQSKLDSPEELEPDREEDSSSCIDIQEVLSSSESDS | |
| | CNSSSSDIIRDLLEEEEAWEASHKNDLQDTEISPRQNRRTRAAESAEIEPRNKRN | |
| | RN | |
| RBMX | MVEADRPGKLFIGGLNTETNEKALEAVFGKYGRIVEVLLMKDRETNKSRGFAF | 717 |
| | VTFESPADAKDAARDMNGKSLDGKAIKVEQATKPSFESGRRGPPPPPRSRGPPR | |
| | GLRGGRGGSGGTRGPPSRGGHMDDGGYSMNFNMSSSRGPLPVKRGPPPRSGGP | |
| | PPKRSAPSGPVRSSSGMGGRAPVSRGRDSYGGPPRREPLPSRRDVYLSPRDDGY | |
| | STKDSYSSRDYPSSRDTRDYAPPPRDYTYRDYGHSSSRDDYPSRGYSDRDGYGR | |
| | DRDYSDHPSGGSYRDSYESYGNSRSAPPTRGPPPSYGGSSRYDDYSSSRDGYGG | |
| | SRDSYSSSRSDLYSSGRDRVGRQERGLPPSMERGYPPPRDSYSSSSRGAPRGGGR | |
| | GGSRSDRGGGRSRY | |
| REV1 | MRRGGWRKRAENDGWETWGGYMAAKVQKLEEQFRSDAAMQKDGTSSTIFSG | 718 |
| | VAIYVNGYTDPSAEELRKLMMLHGGQYHVYYSRSKTTHIIATNLPNAKIKELKG | |
| | EKVIRPEWIVESIKAGRLLSYIPYQLYTKQSSVQKGLSFNPVCRPEDPLPGPSNIA | |
| | KQLNNRVNHIVKKIETENEVKVNGMNSWNEEDENNDFSFVDLEQTSPGRKQN | |
| | GIPHPRGSTAIFNGHTPSSNGALKTQDCLVPMVNSVASRLSPAFSQEEDKAEKSS | |
| | TDFRDCTLQQLQQSTRNTDALRNPHRTNSFSLSPLHSNTKINGAHHSTVQGPSST | |
| | KSTSSVSTFSKAAPSVPSKPSDCNFISNFYSHSRLHHISMWKCELTEFVNTLQRQS | |
| | NGIFPGREKLKKMKTGRSALVVTDTGDMSVLNSPRHQSCIMHVDMDCFFVSVG | |
| | IRNRPDLKGKPVAVTSNRGTGRAPLRPGANPQLEWQYYQNKILKGKAADIPDSS | |
| | LWENPDSAQANGIDSVLSRAELASCSYEARQLGIKNGMFFGHAKQLCPNLQAVP | |
| | YDFHAYKEVAQTLYETLASYTHNIEAVSCDEALVDITELLAETKLTPDEFANAV | |
| | RMEIKDQTKCAASVGIGSNILLARMATRKAKPDGQYHLKPEEVDDFIRGQLVT | |
| | NLPGVGHSMESKLASLGIKTCGDLQYMTMAKLQKEFGPKTGQMLYRFCRGLD | |
| | DRPVRTEKERKSVSAEINYGIRFTQPKEAEAFLLSLSEEIQRRLEATGMKGKRLT | |
| | LKIMVRKPGAPVETAKFGGHGICDNIARTVTLDQATDNAKIIGKAMLNMFHTM | |
| | KLNISDMRGVGIHVNQLVPTNLNPSTCPSRPSVQSSHFPSGSYSVRDVFQVQKA | |
| | KKSTEEEHKEVFRAAVDLEISSASRTCTFLPPFPAHLPTSPDTNKAESSGKWNGL | |
| | HTPVSVQSRLNLSIEVPSPSQLDQSVLEALPPDLREQVEQVCAVQQAESHGDKK | |
| | KEPVNGCNTGILPQPVGTVLLQIPEPQESNSDAGINLIALPAFSQVDPEVFAALPA | |
| | ELQRELKAAYDQRQRQGENSTHQQSASASVPKNPLLHLKAAVKEKKRNKKKK | |
| | TIGSPKRIQSPLNNKLLNSPAKTLPGACGSPQKLIDGFLKHEGPPAEKPLEELSAS | |
| | TSGVPGLSSLQSDPAGCVRPPAPNLAGAVEFNDVKTLLREWITTISDPMEEDILQ | |
| | VVKYCTDLIEEKDLEKLDLVIKYMKRLMQQSVESVWNMAFDFILDNVQVVLQ | |
| | QTYGSTLKVT | |
| RFWD3 | MAHEAMEYDVQVQLNHAEQQPAPAGMASSQGGPALLQPVPADVVSSQGVPSI | 719 |
| | LQPAPAEVISSQATPPLLQPAPQLSVDLTEVEVLGEDTVENINPRTSEQHRQGSD | |
| | GNHTIPASSLHSMTNFISGLQRLHGMLEFLRPSSSNHSVGPMRTRRRVSASRRAR | |
| | AGGSQRTDSARLRAPLDAYFQVSRTQPDLPATTYDSETRNPVSEELQVSSSSDS | |
| | DSDSSAEYGGVVDQAEESGAVILEEQLAGVSAEQEVTCIDGGKTLPKQPSPQKS | |
| | EPLLPSASMDEEEGDTCTICLEQWTNAGDHRLSALRCGHLFGYRCISTWLKGQV | |
| | RKCPQCNKKARHSDIVVLYARTLRALDTSEQERMKSSLLKEQMLRKQAELESA | |
| | QCRLQLQVLTDKCTRLQRRVQDLQKLTSHQSQNLQQPRGSQAWVLSCSPSSQG | |
| | QHKHKYHFQKTFTVSQAGNCRIMAYCDALSCLVISQPSPQASFLPGFGVKMLST | |
| | ANMKSSQYIPMHGKQIRGLAFSSYLRGLLLSASLDNTIKLTSLETNTVVQTYNA | |
| | GRPVWSCCWCLDEANYIYAGLANGSILVYDVRNTSSHVQELVAQKARCPLVSL | |
| | SYMPRAASAAFPYGGVLAGTLEDASFWEQKMDFSHWPHVLPLEPGGCIDFQTE | |
| | NSSRHCLVTYRPDKNHTTIRSVLMEMSYRLDDTGNPICSCQPVHTFFGGPTCKL | |
| | LTKNAIFQSPENDGNILVCTGDEAANSALLWDAASGSLLQDLQTDQPVLDICPF | |
| | EVNRNSYLATLTEKMVHIYKWE | |
| TIMELESS | MDLHMMNCELLATCSALGYLEGDTYHKEPDCLESVKDLIRYLRHEDETRDVR | 720 |
| | QQLGAAQILQSDLLPILTQHHQDKPLFDAVIRLMVNLTQPALLCFGNLPKEPSFR | |
| | HHFLQVLTYLQAYKEAFASEKAFGVLSETLYELLQLGWEERQEEDNLLIERILL | |
| | LVRNILHVPADLDQEKKIDDDASAHDQLLWAIHLSGLDDLLLFLASSSAEEQWS | |
| | LHVLEIVSLMFRDQNPEQLAGVGQGRLAQERSADFAELEVLRQREMAEKKTRA | |
| | LQRGNRHSRFGGSYIVQGLKSIGERDLIFHKGLHNLRNYSSDLGKQPKKVPKRR | |
| | QAARELSIQRRSALNVRLFLRDFCSEFLENCYNRLMGSVKDHLLREKAQQHDE | |
| | TYYMWALAFFMAFNRAASFRPGLVSETLSVRTFHFIEQNLTNYYEMMLTDRKE | |
| | AASWARRMHLALKAYQELLATVNEMDISPDEAVRESSRIIKNNIFYVMEYRELF | |
| | LALFRKFDERCQPRSFLRDLVETTHLFLKMLERFCRSRGNLVVQNKQKKRRKK | |
| | KKKVLDQAIVSGNVPSSPEEVEAVWPALAEQLQCCAQNSELSMDSVVPFDAAS | |
| | EVPVEEQRAEAMVRIQDCLLAGQAPQALTLLRSAREVWPEGDVFGSQDISPEEE | |
| | IQLLKQILSAPLPRQQGPEERGAEEEEEEEEEEEEELQVVQVSEKEFNFLDYLKRF | |
| | ACSTVVRAYVLLLRSYQQNSAHTNHCIVKMLHRLAHDLKMEALLFQLSVFCLF | |
| | NRLLSDPAAGAYKELVTFAKYILGKFFALAAVNQKAFVELLFWKNTAVVREM | |
| | TEGYGSLDDRSSSRRAPTWSPEEEAHLRELYLANKDVEGQDVVEAILAHLNTVP | |
| | RTRKQIIHHLVQMGLADSVKDFQRKGTHIVLWTGDQELELQRLFEEFRDSDDV | |
| | LGHIMKNITAKRSRARIVDKLLALGLVAERRELYKKRQKKLASSILPNGAESLK | |
| | DFCQEDLEEEENLPEEDSEEEEEGGSEAEQVQGSLVLSNENLGQSLHQEGFSIPL | |
| | LWLQNCLIRAADDREEDGCSQAVPLVPLTEENEEAMENEQFQQLLRKLGVRPP | |
| | ASGQETFWRIPAKLSPTQLRRAAASLSQPEEEQKLQPELQPKVPGEQGSDEEHC | |
| | KEHRAQALRALLLAHKKKAGLASPEEEDAVGKEPLKAAPKKRQLLDSDEEQEE | |
| | DEGRNRAPELGAPGIQKKKRYQIEDDEDD | |
| UBE2I | MSGIALSRLAQERKAWRKDHPFGFVAVPTKNPDGTMNLMNWECAIPGKKGTP | 721 |
| | WEGGLFKLRMLFKDDYPSSPPKCKFEPPLFHPNVYPSGTVCLSILEEDKDWRPAI | |
| | TIKQILLGIQELLNEPNIQDPAQAEAYTIYCQNRVEYEKRVRAQAKKFAPS | |
| UBE2T | MQRASRLKRELHMLATEPPPGITCWQDKDQMDDLRAQILGGANTPYEKGVFK | 722 |
| | LEVIIPERYPFEPPQIRFLTPIYHPNIDSAGRICLDVLKLPPKGAWRPSLNIATVLTS | |
| | IQLLMSEPNPDDPLMADISSEFKYNKPAFLKNARQWTEKHARQKQKADEEEML | |
| | DNLPEAGDSRVHNSTQKRKASQLVGIEKKFHPDV | |
| UNG | MIGQKTLYSFFSPSPARKRHAPSPEPAVQGTGVAGVPEESGDAAAIPAKKAPAG | 723 |
| | QEEPGTPPSSPLSAEQLDRIQRNKAAALLRLAARNVPVGFGESWKKHLSGEFGK | |
| | PYFIKLMGFVAEERKHYTVYPPPHQVFTWTQMCDIKDVKVVILGQDPYHGPNQ | |
| | AHGLCFSVQRPVPPPPSLENIYKELSTDIEDFVHPGHGDLSGWAKQGVLLLNAV | |
| | LTVRAHQANSHKERGWEQFTDAVVSWLNQNSNGLVFLLWGSYAQKKGSAIDR | |
| | KRHHVLQTAHPSPLSVYRGFFGCRHFSKTNELLQKSGKKPIDWKEL | |

Cas9 effector domains

| | | |
|---|---|---|
| SpCas9 | MDKKYSIGLDIGTNSVGWAVITDEYKVPSKKFKVLGNTDRHSIKKNLIGALLFD | 724 |
| | SGETAEATRLKRTARRRYTRRKNRICYLQEIFSNEMAKVDDSFFHRLEESFLVEE | |
| | DKKHERHPIFGNIVDEVAYHEKYPTIYHLRKKLVDSTDKADLRLIYLALAHMIK | |
| | FRGHFLIEGDLNPDNSDVDKLFIQLVQTYNQLFEENPINASGVDAKAILSARLSK | |
| | SRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSD | |
| | NVPSEEVVKKMKNYWRQLLNAKLITQRKFDNLTKAERGGLSELDKAGFIKRQL | |
| | VETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQE | |
| | IGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSP | |
| | TVAYSVLVVAKVEKGKSKKLKSVKELLGITIMERSSFEKNPIDFLEAKGYKEVK | |
| | KDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGL | |
| | YETRIDLSQLGGD | |
| HF-SpCas9n (D10A, | MDKKYSIGLAIGTNSVGWAVITDEYKVPSKKFKVLGNTDRHSIKKNLIGALLFD | 725 |
| N497A, R661A, | SGETAEATRLKRTARRRYTRRKNRICYLQEIFSNEMAKVDDSFFHRLEESFLVEE | |
| Q695A, Q926A) | DKKHERHPIFGNIVDEVAYHEKYPTIYHLRKKLVDSTDKADLRLIYLALAHMIK | |
| | FRGHFLIEGDLNPDNSDVDKLFIQLVQTYNQLFEENPINASGVDAKAILSARLSK | |
| | SRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTAFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGALSRKLINGIRDKQSGKTILDFLKSDGFANRNFMALIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSD | |
| | NVPSEEVVKKMKNYWRQLLNAKLITQRKFDNLTKAERGGLSELDKAGFIKRQL | |
| | VETRAITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQE | |
| | IGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSP | |
| | TVAYSVLVVAKVEKGKSKKLKSVKELLGITIMERSSFEKNPIDFLEAKGYKEVK | |
| | KDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGL | |
| | YETRIDLSQLGGD | |
| e-SpCas9 (K848A, | MDKKYSIGLDIGTNSVGWAVITDEYKVPSKKFKVLGNTDRHSIKKNLIGALLFD | 726 |
| K1003A, R1060A) | SGETAEATRLKRTARRRYTRRKNRICYLQEIFSNEMAKVDDSFFHRLEESFLVEE | |
| | DKKHERHPIFGNIVDEVAYHEKYPTIYHLRKKLVDSTDKADLRLIYLALAHMIK | |
| | FRGHFLIEGDLNPDNSDVDKLFIQLVQTYNQLFEENPINASGVDAKAILSARLSK | |
| | SRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLADDSIDNKVLTRSDKNRGKSD | |
| | NVPSEEVVKKMKNYWRQLLNAKLITQRKFDNLTKAERGGLSELDKAGFIKRQL | |
| | VETRQITKHVAQILDSRMNTKYDENDKLIREVKVITLKSKLVSDFRKDFQFYKV | |
| | REINNYHHAHDAYLNAVVGTALIKKYPALESEFVYGDYKVYDVRKMIAKSEQE | |
| | IGKATAKYFFYSNIMNFFKTEITLANGEIRKAPLIETNGETGEIVWDKGRDFATV | |
| | RKVLSMPQVNIVKKTEVQTGGFSKESILPKRNSDKLIARKKDWDPKKYGGFDSP | |
| | TVAYSVLVVAKVEKGKSKKLKSVKELLGITIMERSSFEKNPIDFLEAKGYKEVK | |
| | KDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGL | |
| | YETRIDLSQLGGD | |
| Hypa-Cas9 (N692A, | MDKKYSIGLDIGTNSVGWAVITDEYKVPSKKFKVLGNTDRHSIKKNLIGALLFD | 727 |
| M694A, Q695A, | SGETAEATRLKRTARRRYTRRKNRICYLQEIFSNEMAKVDDSFFHRLEESFLVEE | |
| D1135E) | DKKHERHPIFGNIVDEVAYHEKYPTIYHLRKKLVDSTDKADLRLIYLALAHMIK | |
| | FRGHFLIEGDLNPDNSDVDKLFIQLVQTYNQLFEENPINASGVDAKAILSARLSK | |
| | SRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRAFAALIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSD | |
| | NVPSEEVVKKMKNYWRQLLNAKLITQRKFDNLTKAERGGLSELDKAGFIKRQL | |
| | VETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQE | |
| | IGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFESP | |
| | TVAYSVLVVAKVEKGKSKKLKSVKELLGITIMERSSFEKNPIDFLEAKGYKEVK | |
| | KDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGL | |
| | YETRIDLSQLGGD | |
| Hypa-nCas9 (D10A, | MDKKYSIGLDIGTNSVGWAVITDEYKVPSKKFKVLGNTDRHSIKKNLIGALLFD | 728 |
| N692A, M694A, | SGETAEATRLKRTARRRYTRRKNRICYLQEIFSNEMAKVDDSFFHRLEESFLVEE | |
| Q695A, D1135E) | DKKHERHPIFGNIVDEVAYHEKYPTIYHLRKKLVDSTDKADLRLIYLALAHMIK | |
| | FRGHFLIEGDLNPDNSDVDKLFIQLVQTYNQLFEENPINASGVDAKAILSARLSK | |
| | SRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRAFAALIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSD | |
| | NVPSEEVVKKMKNYWRQLLNAKLITQRKFDNLTKAERGGLSELDKAGFIKRQL | |
| | VETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQE | |
| | IGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFESP | |
| | TVAYSVLVVAKVEKGKSKKLKSVKELLGITIMERSSFEKNPIDFLEAKGYKEVK | |
| | KDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGL | |
| | YETRIDLSQLGGD | |
| e-HF-SpCas9n (D10A, | MDKKYSIGLAIGTNSVGWAVITDEYKVPSKKFKVLGNTDRHSIKKNLIGALLFD | 729 |
| K848A, K1003A, | SGETAEATRLKRTARRRYTRRKNRICYLQEIFSNEMAKVDDSFFHRLEESFLVEE | |
| R1060A//N497A, | DKKHERHPIFGNIVDEVAYHEKYPTIYHLRKKLVDSTDKADLRLIYLALAHMIK | |
| R661A, Q695A, | FRGHFLIEGDLNPDNSDVDKLFIQLVQTYNQLFEENPINASGVDAKAILSARLSK | |
| Q926A) | SRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTAFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGALSRKLINGIRDKQSGKTILDFLKSDGFANRNFMALIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLADDSIDNKVLTRSDKNRGKSD | |
| | NVPSEEVVKKMKNYWRQLLNAKLITQRKFDNLTKAERGGLSELDKAGFIKRQL | |
| | VETRAITKHVAQILDSRMNTKYDENDKLIREVKVITLKSKLVSDFRKDFQFYKV | |
| | REINNYHHAHDAYLNAVVGTALIKKYPALESEFVYGDYKVYDVRKMIAKSEQE | |
| | IGKATAKYFFYSNIMNFFKTEITLANGEIRKAPLIETNGETGEIVWDKGRDFATV | |
| | RKVLSMPQVNIVKKTEVQTGGFSKESILPKRNSDKLIARKKDWDPKKYGGFDSP | |
| | TVAYSVLVVAKVEKGKSKKLKSVKELLGITIMERSSFEKNPIDFLEAKGYKEVK | |
| | KDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGL | |
| | YETRIDLSQLGGD | |
| e-Hypa-SpCas9n | MDKKYSIGLAIGTNSVGWAVITDEYKVPSKKFKVLGNTDRHSIKKNLIGALLFD | 730 |
| (D10A, K848A, | SGETAEATRLKRTARRRYTRRKNRICYLQEIFSNEMAKVDDSFFHRLEESFLVEE | |
| K1003A, | DKKHERHPIFGNIVDEVAYHEKYPTIYHLRKKLVDSTDKADLRLIYLALAHMIK | |
| R1060A//N692A, | FRGHFLIEGDLNPDNSDVDKLFIQLVQTYNQLFEENPINASGVDAKAILSARLSK | |
| M694A, Q695A, | SRRLENLIAQLPGEKKNGLFGNLIALSLGLTPNFKSNFDLAEDAKLQLSKDTYD | |
| D1135E) | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | TERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRAFAALIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLADDSIDNKVLTRSDKNRGKSD | |
| | NVPSEEVVKKMKNYWRQLLNAKLITQRKFDNLTKAERGGLSELDKAGFIKRQL | |
| | VETRQITKHVAQILDSRMNTKYDENDKLIREVKVITLKSKLVSDFRKDFQFYKV | |
| | REINNYHHAHDAYLNAVVGTALIKKYPALESEFVYGDYKVYDVRKMIAKSEQE | |
| | IGKATAKYFFYSNIMNFFKTEITLANGEIRKAPLIETNGETGEIVWDKGRDFATV | |
| | RKVLSMPQVNIVKKTEVQTGGFSKESILPKRNSDKLIARKKDWDPKKYGGFESP | |
| | TVAYSVLVVAKVEKGKSKKLKSVKELLGITIMERSSFEKNPIDFLEAKGYKEVK | |
| | KDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGL | |
| | YETRIDLSQLGGD | |
| HF-Hypa-SpCas9n | MDKKYSIGLAIGTNSVGWAVITDEYKVPSKKFKVLGNTDRHSIKKNLIGALLFD | 731 |
| (D10A, N497A, | SGETAEATRLKRTARRRYTRRKNRICYLQEIFSNEMAKVDDSFFHRLEESFLVEE | |
| R661A, Q695A, | DKKHERHPIFGNIVDEVAYHEKYPTIYHLRKKLVDSTDKADLRLIYLALAHMIK | |
| Q926A//N692A, | FRGHFLIEGDLNPDNSDVDKLFIQLVQTYNQLFEENPINASGVDAKAILSARLSK | |
| M694A, Q695A, | SRRLENLIAQLPGEKKNGLFGNLIALSLGLTPNFKSNFDLAEDAKLQLSKDTYD | |
| D1135E) | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTAFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRAFAALIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSD | |
| | NVPSEEVVKKMKNYWRQLLNAKLITQRKFDNLTKAERGGLSELDKAGFIKRQL | |
| | VETRAITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQE | |
| | IGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFESP | |
| | TVAYSVLVVAKVEKGKSKKLKSVKELLGITIMERSSFEKNPIDFLEAKGYKEVK | |
| | KDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGL | |
| | YETRIDLSQLGGD | |
| e-HF-Hypa-SpCas9n | MDKKYSIGLAIGTNSVGWAVITDEYKVPSKKFKVLGNTDRHSIKKNLIGALLFD | 732 |
| (D10A, K848A, | SGETAEATRLKRTARRRYTRRKNRICYLQEIFSNEMAKVDDSFFHRLEESFLVEE | |
| K1003A, R1060A// | DKKHERHPIFGNIVDEVAYHEKYPTIYHLRKKLVDSTDKADLRLIYLALAHMIK | |
| //N497A, R661A, | FRGHFLIEGDLNPDNSDVDKLFIQLVQTYNQLFEENPINASGVDAKAILSARLSK | |
| Q695A, | SRRLENLIAQLPGEKKNGLFGNLIALSLGLTPNFKSNFDLAEDAKLQLSKDTYD | |
| Q926A//N692A, | DDLDNLLAQIGDQYADLFLAAKNLSDAILLSDILRVNTEITKAPLSASMIKRYDE | |
| M694A, Q695A, | HHQDLTLLKALVRQQLPEKYKEIFFDQSKNGYAGYIDGGASQEEFYKFIKPILEK | |
| D1135E) * | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTAFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRAFAALIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLADDSIDNKVLTRSDKNRGKSD | |
| | NVPSEEVVKKMKNYWRQLLNAKLITQRKFDNLTKAERGGLSELDKAGFIKRQL | |
| | VETRAITKHVAQILDSRMNTKYDENDKLIREVKVITLKSKLVSDFRKDFQFYKV | |
| | REINNYHHAHDAYLNAVVGTALIKKYPALESEFVYGDYKVYDVRKMIAKSEQE | |
| | IGKATAKYFFYSNIMNFFKTEITLANGEIRKAPLIETNGETGEIVWDKGRDFATV | |
| | RKVLSMPQVNIVKKTEVQTGGFSKESILPKRNSDKLIARKKDWDPKKYGGFESP | |
| | TVAYSVLVVAKVEKGKSKKLKSVKELLGITIMERSSFEKNPIDFLEAKGYKEVK | |
| | KDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGL | |
| | YETRIDLSQLGGD | |
| Sniper-nCas9 (D10A, | MDKKYSIGLAIGTNSVGWAVITDEYKVPSKKFKVLGNTDRHSIKKNLIGALLFD | 733 |
| F539S, M763I, K890N) | SGETAEATRLKRTARRRYTRRKNRICYLQEIFSNEMAKVDDSFFHRLEESFLVEE | |
| | DKKHERHPIFGNIVDEVAYHEKYPTIYHLRKKLVDSTDKADLRLIYLALAHMIK | |
| | FRGHFLIEGDLNPDNSDVDKLFIQLVQTYNQLFEENPINASGVDAKAILSARLSK | |
| | SRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPASLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIE | |
| | IARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDN | |
| | VPSEEVVKKMKNYWRQLLNANLITQRKFDNLTKAERGGLSELDKAGFIKRQLV | |
| | ETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEI | |
| | GKATAKYFFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKYGGFDSPT | |
| | VAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNKHRDKP | |
| | IREQAENIIHLFTLTNLGAPAAFKYFDTTIDRKRYTSTKEVLDATLIHQSITGLYE | |
| | TRIDLSQLGGD | |
| Cas9-NG (L111R, | MDKKYSIGLDIGTNSVGWAVITDEYKVPSKKFKVLGNTDRHSIKKNLIGALLFD | 734 |
| D1135V, G1218R, | SGETAEATRLKRTARRRYTRRKNRICYLQEIFSNEMAKVDDSFFHRLEESFLVEE | |
| E1219F, A1322R, | DKKHERHPIFGNIVDEVAYHEKYPTIYHLRKKLVDSTDKADLRLIYLALAHMIK | |
| R1335V, T1337R) | FRGHFLIEGDLNPDNSDVDKLFIQLVQTYNQLFEENPINASGVDAKAILSARLSK | |
| | SRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSD | |
| | NVPSEEVVKKMKNYWRQLLNAKLITQRKFDNLTKAERGGLSELDKAGFIKRQL | |
| | VETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQE | |
| | IGKATAKYFFYSNIMNFFKTEITLANGEIRKRPLIETNGETGEIVWDKGRDFATV | |
| | RKVLSMPQVNIVKKTEVQTGGFSKESIRPKRNSDKLIARKKDWDPKKYGGFVSP | |
| | TVAYSVLVVAKVEKGKSKKLKSVKELLGITIMERSSFEKNPIDFLEAKGYKEVK | |
| | KDLIIKLPKYSLFELENGRKRMLASARFLQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPRAFKYFDTTIDRKVYRSTKEVLDATLIHQSITGL | |
| | YETRIDLSQLGGD | |
| nCas9-NG (D10A, | MDKKYSIGLAIGTNSVGWAVITDEYKVPSKKFKVLGNTDRHSIKKNLIGALLFD | 735 |
| L111R, D1135V, | SGETAEATRLKRTARRRYTRRKNRICYLQEIFSNEMAKVDDSFFHRLEESFLVEE | |
| G1218R, E1219F, | DKKHERHPIFGNIVDEVAYHEKYPTIYHLRKKLVDSTDKADLRLIYLALAHMIK | |
| A1322R, R1335V, | FRGHFLIEGDLNPDNSDVDKLFIQLVQTYNQLFEENPINASGVDAKAILSARLSK | |
| T1337R) | SRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDE | |
| | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | TERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSD | |
| | NVPSEEVVKKMKNYWRQLLNAKLITQRKFDNLTKAERGGLSELDKAGFIKRQL | |
| | VETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQE | |
| | IGKATAKYFFYSNIMNFFKTEITLANGEIRKRPLIETNGETGEIVWDKGRDFATV | |
| | RKVLSMPQVNIVKKTEVQTGGFSKESIRPKRNSDKLIARKKDWDPKKYGGFVSP | |
| | TVAYSVLVVAKVEKGKSKKLKSVKELLGITIMERSSFEKNPIDFLEAKGYKEVK | |
| | KDLIIKLPKYSLFELENGRKRMLASARFLQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPRAFKYFDTTIDRKVYRSTKEVLDATLIHQSITGL | |
| | YETRIDLSQLGGD | |
| HF-nCas9-NG (D10A, | MDKKYSIGLAIGTNSVGWAVITDEYKVPSKKFKVLGNTDRHSIKKNLIGALLFD | 736 |
| N497A, R661A, | SGETAEATRLKRTARRRYTRRKNRICYLQEIFSNEMAKVDDSFFHRLEESFLVEE | |
| Q695A, | DKKHERHPIFGNIVDEVAYHEKYPTIYHLRKKLVDSTDKADLRLIYLALAHMIK | |
| Q926A//L111R, | FRGHFLIEGDLNPDNSDVDKLFIQLVQTYNQLFEENPINASGVDAKAILSARLSK | |
| D1135V, G1218R, | SRRLENLIAQLPGEKKNGLFGNLIALSLGLTPNFKSNFDLAEDAKLQLSKDTYD | |
| E1219F, A1322R, | DDLDNLLAQIGDQYADLFLAAKNLSDAILLSDILRVNTEITKAPLSASMIKRYDE | |
| R1335V, T1337R//) | HHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTAFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGALSRKLINGIRDKQSGKTILDFLKSDGFANRNFMALIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSD | |
| | NVPSEEVVKKMKNYWRQLLNAKLITQRKFDNLTKAERGGLSELDKAGFIKRQL | |
| | VETRAITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQE | |
| | IGKATAKYFFYSNIMNFFKTEITLANGEIRKRPLIETNGETGEIVWDKGRDFATV | |
| | RKVLSMPQVNIVKKTEVQTGGFSKESIRPKRNSDKLIARKKDWDPKKYGGFVSP | |
| | TVAYSVLVVAKVEKGKSKKLKSVKELLGITIMERSSFEKNPIDFLEAKGYKEVK | |
| | KDLIIKLPKYSLFELENGRKRMLASARFLQKGNELALPSKYVNFLYLASHYEKL | |
| | KGSPEDNEQKQLFVEQHKHYLDEIIEQISEFSKRVILADANLDKVLSAYNKHRD | |
| | KPIREQAENIIHLFTLTNLGAPRAFKYFDTTIDRKVYRSTKEVLDATLIHQSITGL | |
| | YETRIDLSQLGGD | |

Cellular DNA polymerase domains

| | | |
|---|---|---|
| PolB | MSKRKAPQETLNGGITDMLTELANFEKNVSQAIHKYNAYRKAASVIAK | 737 |
| | YPHKIKSGAEAKKLPGVGTKIAEKIDEFLATGKLRKLEKIRQDDTSSSIN | |
| | FLTRVSGIGPSAARKFVDEGIKTLEDLRKNEDKLNHHQRIGLKYFGDFE | |
| | KRIPREEMLQMQDIVLNEVKKVDSEYIATVCGSFRRGAESSGDMDVLL | |
| | THPSFTSESTKQPKLLHQVVEQLQKVHFITDTLSKGETKFMGVCQLPSK | |
| | NDEKEYPHRRIDIRLIPKDQYYCGVLYFTGSDIFNKNMRAHALEKGFTI | |
| | NEYTIRPLGVTGVAGEPLPVDSEKDIFDYIQWKYREPKDRSE | |
| PolD1 | MDGKRRPGPGPGVPPKRARGGLWDDDDAPRPSQFEEDLALMEEMEAE | 738 |
| | HRLQEQEEEELQSVLEGVADGQVPPSAIDPRWLRPTPPALDPQTEPLIFQ | |
| | QLEIDHYVGPAQPVPGGPPPSHGSVPVLRAFGVTDEGFSVCCHIHGFAP | |
| | YFYTPAPPGFGPEHMGDLQRELNLAISRDSRGGRELTGPAVLAVELCSR | |
| | ESMFGYHGHGPSPFLRITVALPRLVAPARRLLEQGIRVAGLGTPSFAPY | |
| | EANVDFEIRFMVDTDIVGCNWLELPAGKYALRLKEKATQCQLEADVL | |
| | WSDVVSHPPEGPWQRIAPLRVLSFDIECAGRKGIFPEPERDPVIQICSLGL | |
| | RWGEPEPFLRLALTLRPCAPILGAKVQSYEKEEDLLQAWSTFIRIMDPD | |
| | VITGYNIQNFDLPYLISRAQTLKVQTFPFLGRVAGLCSNIRDSSFQSKQT | |
| | GRRDTKVVSMVGRVQMDMLQVLLREYKLRSYTLNAVSFHFLGEQKE | |
| | DVQHSIITDLQNGNDQTRRRLAVYCLKDAYLPLRLLERLMVLVNAVE | |
| | MARVTGVPLSYLLSRGQQVKVVSQLLRQAMHEGLLMPVVKSEGGED | |
| | YTGATVIEPLKGYYDVPIATLDFSSLYPSIMMAHNLCYTTLLRPGTAQK | |
| | LGLTEDQFIRTPTGDEFVKTSVRKGLLPQILENLLSARKRAKAELAKET | |
| | DPLRRQVLDGRQLALKVSANSVYGFTGAQVGKLPCLEISQSVTGFGRQ | |
| | MIEKTKQLVESKYTVENGYSTSAKVVYGDTDSVMCRFGVSSVAEAMA | |
| | LGREAADWVSGHFPSPIRLEFEKVYFPYLLISKKRYAGLLFSSRPDAHD | |
| | RMDCKGLEAVRRDNCPLVANLVTASLRRLLIDRDPEGAVAHAQDVISD | |
| | LLCNRIDISQLVITKELTRAASDYAGKQAHVELAERMRKRDPGSAPSLG | |
| | DRVPYVIISAAKGVAAYMKSEDPLFVLEHSLPIDTQYYLEQQLAKPLLR | |
| | IFEPILGEGRAEAVLLRGDHTRCKTVLTGKVGGLLAFAKRRNCCIGCRT | |
| | VLSHQGAVCEFCQPRESELYQKEVSHLNALEERFSRLWTQCQRCQGSL | |
| | HEDVICTSRDCPIFYMRKKVRKDLEDQEQLLRRFGPPGPEAW | |
| PolH | MATGQDRVVALVDMDCFFVQVEQRQNPHLRNKPCAVVQYKSWKGG | 739 |
| | GIIAVSYEARAFGVTRSMWADDAKKLCPDLLLAQVRESRGKANLTKY | |
| | REASVEVMEIMSRFAVIERASIDEAYVDLTSAVQERLQKLQGQPISADL | |
| | LPSTYIEGLPQGPTTAEETVQKEGMRKQGLFQWLDSLQIDNLTSPDLQL | |
| | TVGAVIVEEMRAAIERETGFQCSAGISHNKVLAKLACGLNKPNRQTLV | |
| | SHGSVPQLFSQMPIRKIRSLGGKLGASVIEILGIEYMGELTQFTESQLQSH | |
| | FGEKNGSWLYAMCRGIEHDPVKPRQLPKTIGCSKNFPGKTALATREQV | |
| | QWWLLQLAQELEERLTKDRNDNDRVATQLVVSIRVQGDKRLSSLRRC | |
| | CALTRYDAHKMSHDAFTVIKNCNTSGIQTEWSPPLTMLFLCATKFSAS | |
| | APSSSTDITSFLSSDPSSLPKVPVTSSEAKTQGSGPAVTATKKATTSLESF | |
| | FQKAAERQKVKEASLSSLTAPTQAPMSNSPSKPSLPFQTSQSTGTEPFFK | |
| | QKSLLLKQKQLNNSSVSSPQQNPWSNCKALPNSLPTEYPGCVPVCEGV | |
| | SKLEESSKATPAEMDLAHNSQSMHASSASKSVLEVTQKATPNPSLLAA | |
| | EDQVPCEKCGSLVPVWDMPEHMDYHFALELQKSFLQPHSSNPQVVSA | |
| | VSHQGKRNPKSPLACTNKRPRPEGMQTLESFFKPLTH | |
| PolI | MEKLGVEPEEEGGGDDDEEDAEAWAMELADVGAAASSQGVHDQVLP | 740 |
| | TPNASSRVIVHVDLDCFYAQVEMISNPELKDKPLGVQQKYLVVTCNYE | |
| | ARKLGVKKLMNVRDAKEKCPQLVLVNGEDLTRYREMSYKVTELLEEF | |
| | SPVVERLGFDENFVDLTEMVEKRLQQLQSDELSAVTVSGHVYNNQSIN | |
| | LLDVLHIRLLVGSQIAAEMREAMYNQLGLTGCAGVASNKLLAKLVSG | |
| | VFKPNQQTVLLPESCQHLIHSLNHIKEIPGIGYKTAKCLEALGINSVRDL | |
| | QTFSPKILEKELGISVAQRIQKLSFGEDNSPVILSGPPQSFSEEDSFKKCSS | |
| | EVEAKNKIEELLASLLNRVCQDGRKPHTVRLIIRRYSSEKHYGRESRQC | |
| | PIPSHVIQKLGTGNYDVMTPMVDILMKLFRNMVNVKMPFHLTLLSVCF | |
| | CNLKALNTAKKGLIDYYLMPSLSTTSRSGKHSFKMKDTHMEDFPKDKE | |
| | TNRDFLPSGRIESTRTRESPLDTTNFSKEKDINEFPLCSLPEGVDQEVFKQ | |
| | LPVDIQEEILSGKSREKFQGKGSVSCPLHASRGVLSFFSKKQMQDIPINP | |
| | RDHLSSSKQVSSVSPCEPGTSGFNSSSSSYMSSQKDYSYYLDNRLKDER | |
| | ISQGPKEPQGFHFTNSNPAVSAFHSFPNLQSEQLFSRNHTTDSHKQTVA | |
| | TDSHEGLTENREPDSVDEKITFPSDIDPQVFYELPEAVQKELLAEWKRA | |
| | GSDFHIGHK | |
| PolK | MDSTKEKCDSYKDDLLLRMGLNDNKAGMEGLDKEKINKIIMEATKGS | 741 |
| | RFYGNELKKEKQVNQRIENMMQQKAQITSQQLRKAQLQVDRFAMELE | |
| | QSRNLSNTIVHIDMDAFYAAVEMRDNPELKDKPIAVGSMSMLSTSNYH | |
| | ARRFGVRAAMPGFIAKRLCPQLIIVPPNFDKYRAVSKEVKEILADYDPN | |
| | FMAMSLDEAYLNITKHLEERQNWPEDKRRYFIKMGSSVENDNPGKEV | |
| | NKLSEHERSISPLLFEESPSDVQPPGDPFQVNFEEQNNPQILQNSVVFGTS | |
| | AQEVVKEIRFRIEQKTTLTASAGIAPNTMLAKVCSDKNKPNGQYQILPN | |
| | RQAVMDFIKDLPIRKVSGIGKVTEKMLKALGIITCTELYQQRALLSLLES | |
| | ETSWHYFLHISLGLGSTHLTRDGERKSMSVERTFSEINKAEEQYSLCQE | |
| | LCSELAQDLQKERLKGRTVTIKLKNVNFEVKTRASTVSSVVSTAEEIFAI | |
| | AKELLKTEIDADFPHPLRLRLMGVRISSFPNEEDRKHQQRSIIGFLQAGN | |
| | QALSATECTLEKTDKDKFVKPLEMSHKKSFFDKKRSERKWSHQDTFKC | |
| | EAVNKQSFQTSQPFQVLKKKMNENLEISENSDDCQILTCPVCFRAQGCI | |
| | SLEALNKHVDECLDGPSISENFKMFSCSHVSATKVNKKENVPASSLCEK | |
| | QDYEAHPKIKEISSVDCIALVDTIDNSSKAESIDALSNKHSKEECSSLPSK | |
| | SFNIEHCHQNSSSTVSLENEDVGSFRQEYRQPYLCEVKTGQALVCPVCN | |
| | VEQKTSDLTLFNVHVDVCLNKSFIQELRKDKFNPVNQPKESSRSTGSSS | |
| | GVQKAVTRTKRPGLMTKYSTSKKIKPNNPKHTLDIFFK | |
| REV1 | MRRGGWRKRAENDGWETWGGYMAAKVQKLEEQFRSDAAMQKDGT | 742 |
| | SSTIFSGVAIYVNGYTDPSAEELRKLMMLHGGQYHVYYSRSKTTHIIAT | |
| | NLPNAKIKELKGEKVIRPEWIVESIKAGRLLSYIPYQLYTKQSSVQKGLS | |
| | FNPVCRPEDPLPGPSNIAKQLNNRVNHIVKKIETENEVKVNGMNSWNE | |
| | EDENNDFSFVDLEQTSPGRKQNGIPHPRGSTAIFNGHTPSSNGALKTQD | |
| | CLVPMVNSVASRLSPAFSQEEDKAEKSSTDFRDCTLQQLQQSTRNTDA | |
| | LRNPHRTNSFSLSPLHSNTKINGAHHSTVQGPSSTKSTSSVSTFSKAAPS | |
| | VPSKPSDCNFISNFYSHSRLHHISMWKCELTEFVNTLQRQSNGIFPGREK | |
| | LKKMKTGRSALVVTDTGDMSVLNSPRHQSCIMHVDMDCFFVSVGIRN | |
| | RPDLKGKPVAVTSNRGTGRAPLRPGANPQLEWQYYQNKILKGKAADIP | |
| | DSSLWENPDSAQANGIDSVLSRAEIASCSYEARQLGIKNGMFFGHAKQ | |
| | LCPNLQAVPYDFHAYKEVAQTLYETLASYTHNIEAVSCDEALVDITEIL | |
| | AETKLTPDEFANAVRMEIKDQTKCAASVGIGSNILLARMATRKAKPDG | |
| | QYHLKPEEVDDFIRGQLVTNLPGVGHSMESKLASLGIKTCGDLQYMTM | |
| | AKLQKEFGPKTGQMLYRFCRGLDDRPVRTEKERKSVSAEINYGIRFTQP | |
| | KEAEAFLLSLSEEIQRRLEATGMKGKRLTLKIMVRKPGAPVETAKFGG | |
| | HGICDNIARTVTLDQATDNAKIIGKAMLNMFHTMKLNISDMRGVGIHV | |
| | NQLVPTNLNPSTCPSRPSVQSSHFPSGSYSVRDVFQVQKAKKSTEEEHK | |
| | EVFRAAVDLEISSASRTCTFLPPFPAHLPTSPDTNKAESSGKWNGLHTPV | |
| | SVQSRLNLSIEVPSPSQLDQSVLEALPPDLREQVEQVCAVQQAESHGDK | |
| | KKEPVNGCNTGILPQPVGTVLLQIPEPQESNSDAGINLIALPAFSQVDPE | |
| | VFAALPAELQRELKAAYDQRQRQGENSTHQQSASASVPKNPLLHLKA | |
| | AVKEKKRNKKKKTIGSPKRIQSPLNNKLLNSPAKTLPGACGSPQKLIDG | |
| | FLKHEGPPAEKPLEELSASTSGVPGLSSLQSDPAGCVRPPAPNLAGAVE | |
| | FNDVKTLLREWITTISDPMEEDILQVVKYCTDLIEEKDLEKLDLVIKYM | |
| | KRLMQQSVESVWNMAFDFILDNVQVVLQQTYGSTLKVT | |
| REV3L (catalytic | MGLSPLSTEPKTQKLSNKKGSNTDTLRRVLLTQAKNQFAAVNTPQKET | 743 |
| domain) | SQIDGPSLNNTYGFKVSIQNLQEAKALHEIQNLTLISVELHARTRRDLEP | |
| | DPEFDPICALFYCISSDTPLPDTEKTELTGVIVIDKDKTVFSQDIRYQTPL | |
| | LIRSGITGLEVTYAADEKALFHEIANIIKRYDPDILLGYEIQMHSWGYLL | |
| | QRAAALSIDLCRMISRVPDDKIENRFAAERDEYGSYTMSEINIVGRITLN | |
| | LWRIMRNEVALTNYTFENVSFHVLHQRFPLFTFRVLSDWFDNKTDLYR | |
| | WKMVDHYVSRVRGNLQMLEQLDLIGKTSEMARLFGIQFLHVLTRGSQ | |
| | YRVESMMLRIAKPMNYIPVTPSVQQRSQMRAPQCVPLIMEPESRFYSNS | |
| | VLVLDFQSLYPSIVIAYNYCFSTCLGHVENLGKYDEFKFGCTSLRVPPD | |
| | LLYQVRHDITVSPNGVAFVKPSVRKGVLPRMLEEILKTRFMVKQSMKA | |
| | YKQDRALSRMLDARQLGLKLIANVTFGYTSANFSGRMPCIEVGDSIVH | |
| | KARETLERAIKLVNDTKKWGARVVYGDTDSMFVLLKGATKEQSFKIG | |
| | QEIAEAVTATNPKPVKLKFEKVYLPCVLQTKKRYVGYMYETLDQKDP | |
| | VFDAKGIETVRRDSCPAVSKILERSLKLLFETRDISLIKQYVQRQCMKLL | |
| | EGKASIQDFIFAKEYRGSFSYKPGACVPALELTRKMLTYDRRSEPQVGE | |
| | RVPYVIIYGTPGVPLIQLVRRPVEVLQDPTLRLNATYYITKQILPPLARIF | |
| | SLIGIDVFSWYHELPRIHKATSSSRSEPEGRKGTISQYFTTLHCPVCDDLT | |
| | QHGICSKCRSQPQHVAVILNQEIRELERQQEQLVKICKNCTGCFDRHIPC | |
| | VSLNCPVLFKLSRVNRELSKAPYLRQLLDQF | |
| POlN | MENYEALVGFDLCNTPLSSVAQKIMSAMHSGDLVDSKTWGKSTETME | 744 |
| | VINKSSVKYSVQLEDRKTQSPEKKDLKSLRSQTSRGSAKLSPQSFSVRL | |
| | TDQLSADQKQKSISSLTLSSCLIPQYNQEASVLQKKGHKRKHFLMENIN | |
| | NENKGSINLKRKHITYNNLSEKTSKQMALEEDTDDAEGYLNSGNSGAL | |
| | KKHFCDIRHLDDWAKSQLIEMLKQAAALVITVMYTDGSTQLGADQTP | |
| | VSSVRGIVVLVKRQAEGGHGCPDAPACGPVLEGFVSDDPCIYIQIEHSAI | |
| | WDQEQEAHQQFARNVLFQTMKCKCPVICFNAKDFVRIVLQFFGNDGS | |
| | WKHVADFIGLDPRIAAWLIDPSDATPSFEDLVEKYCEKSITVKVNSTYG | |
| | NSSRNIVNQNVRENLKTLYRLTMDLCSKLKDYGLWQLFRTLELPLIPIL | |
| | AVMESHAIQVNKEEMEKTSALLGARLKELEQEAHFVAGERFLITSNNQ | |
| | LREILFGKLKLHLLSQRNSLPRTGLQKYPSTSEAVLNALRDLHPLPKIILE | |
| | YRQVHKIKSTFVDGLLACMKKGSISSTWNQTGTVTGRLSAKHPNIQGIS | |
| | KHPIQITTPKNFKGKEDKILTISPRAMFVSSKGHTFLAADFSQIELRILTH | |
| | LSGDPELLKLFQESERDDVFSTLTSQWKDVPVEQVTHADREQTKKVVY | |
| | AVVYGAGKERLAACLGVPIQEAAQFLESFLQKYKKIKDFARAAIAQCH | |
| | QTGCVVSIMGRRRPLPRIHAHDQQLRAQAERQAVNFVVQGSAADLCK | |
| | LAMIHVFTAVAASHTLTARLVAQIHDELLFEVEDPQIPECAALVRRTME | |
| | SLEQVQALELQLQVPLKVSLSAGRSWGHLVPLQEAWGPPPGPCRTESP | |
| | SNSLAAPGSPASTQPPPLHFSPSFCL | |
| PolL | MDPRGILKAFPKRQKIHADASSKVLAKIPRREEGEEAEEWLSSLRAHVV | 745 |
| | RTGIGRARAELFEKQIVQHGGQLCPAQGPGVTHIVVDEGMDYERALRL | |
| | LRLPQLPPGAQLVKSAWLSLCLQERRLVDVAGFSIFIPSRYLDHPQPSK | |
| | AEQDASIPPGTHEALLQTALSPPPPPTRPVSPPQKAKEAPNTQAQPISDD | |
| | EASDGEETQVSAADLEALISGHYPTSLEGDCEPSPAPAVLDKWVCAQPS | |
| | SQKATNHNLHITEKLEVLAKAYSVQGDKWRALGYAKAINALKSFHKP | |
| | VTSYQEACSIPGIGKRMAEKIIEILESGHLRKLDHISESVPVLELFSNIWG | |
| | AGTKTAQMWYQQGFRSLEDIRSQASLTTQQAIGLKHYSDFLERMPREE | |
| | ATEIEQTVQKAAQAFNSGLLCVACGSYRRGKATCGDVDVLITHPDGRS | |
| | HRGIFSRLLDSLRQEGFLTDDLVSQEENGQQQKYLGVCRLPGPGRRHR | |
| | RLDIIVVPYSEFACALLYFTGSAHFNRSMRALAKTKGMSLSEHALSTAV | |
| | VRNTHGCKVGPGRVLPTPTEKDVFRLLGLPYREPAERDW | |
| PolI 3M | MVQIPQNPLILVDGSSYLYRAYHAFPPLTNSAGEPTGAMYGVLNMLRS | 746 |
| | LIMQYKPTHAAVVFDAKGKTFRDELFEHYKSHRPPMPDDLRAQIEPLH | |
| | AMVKAMGLPLLAVSGVEADDVIGTLAREAEKAGRPVLISTGDKDMAQ | |
| | LVTPNITLINTMTNTILGPEEVVNKYGVPPELIIDFLALMGDSSDNIPGVP | |
| | GVGEKTAQALLQGLGGLDTLYAEPEKIAGLSFRGAKTMAAKLEQNKE | |
| | VAYLSYQLATIKTDVELELTCEQLEVQQPAAEELLGLFKKYEFKRWTA | |
| | DVEAGKWLQAKGAKPAAKPQETSVADEAPEVTATVISYDNYVTILDEE | |
| | TLKAWIAKLEKAPVFAFDTETDSLDNISANLVGLSFAIEPGVAAYIPVA | |
| | HDYLDAPDQISRERALELLKPLLEDEKALKVGQNLKYDRGILANYGIEL | |
| | RGIAFDTMLESYILNSVAGRHDMDSLAERWLKHKTITFEEIAGKGKNQ | |
| | LTFNQIALEEAGRYAAEDADVTLQLHLKMWPDLQKHKGPLNVFENIE | |
| | MPLVPVLSRIERNGVKIDPKVLHNHSEELTLRLAELEKKAHEIAGEEFN | |
| | LSSTKQLQTILFEKQGIKPLKKTPGGAPSTSEEVLEELALDYPLPKVILEY | |
| | RGLAKLKSTYTDKLPLMINPKTGRVHTSYHQAVTATGRLSSTDPNLQN | |
| | IPVRNEEGRRIRQAFIAPEDYVIVSADYSQIELRIMAHLSRDKGLLTAFA | |
| | EGKDIHRATAAEVFGLPLETVTSEQRRSAKAINFGLIYGMSAFGLARQL | |
| | NIPRKEAQKYMDLYFERYPGVLEYMERTRAQAKEQGYVETLDGRRLY | |
| | LPDIKSSNGARRAAAERAAINAPMQGTAADIIKRAMIAVDAWLQAEQP | |
| | RVRMIMQVHDELVFEVHKDDVDAVAKQIHQLMENCTRLDVPLLVEV | |
| | GSGENWDQAH | |

### REFERENCES FOR EXAMPLE 7

1. Landrum, M. J. et al. ClinVar: public archive of interpretations of clinically relevant variants. Nucleic Acids Res 44, D862-D868 (2016).
2. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
3. Gaudelli, N. M. et al. Programmable base editing of A•T to G•C in genomic DNA without DNA cleavage. Nature 551, 464-471 (2017).
4. Gehrke, J. M. et al. An APOBEC3A-Cas9 base editor with minimized bystander and off-target activities. Nature Biotechnology 36, 977-982 (2018).
5. Nishida, K. et al. Targeted nucleotide editing using hybrid prokaryotic and vertebrate adaptive immune systems. Science 353, aaf8729-aaf8729 (2016).
6. Richter, M. F. et al. Phage-assisted evolution of an adenine base editor with improved Cas domain compatibility and activity. Nature Biotechnology 38, 883-891 (2020).
7. Rees, H. A. & Liu, D. R. Base editing: precision chemistry on the genome and transcriptome of living cells. Nature Reviews Genetics 19, 770-788 (2018).
8. Anzalone, A. V., Koblan, L. W. & Liu, D. R. Genome editing with CRISPR-Cas nucleases, base editors, transposases and prime editors. Nature Biotechnology 38, 824-844 (2020).
9. Gaudelli, N. M. et al. Directed evolution of adenine base editors with increased activity and therapeutic application. Nature Biotechnology 38, 892-900 (2020).
10. Mok, B. Y. et al. A bacterial cytidine deaminase toxin enables CRISPR-free mitochondrial base editing. Nature 583, 631-637 (2020).
11. Komor, A. C. et al. Improved base excision repair inhibition and bacteriophage Mu Gam protein yields C:G-to-T:A base editors with higher efficiency and product purity.
Science Advances 3, eaao4774 (2017).
12. Arbab, M. et al. Determinants of Base Editing Outcomes from Target Library Analysis and Machine Learning. Cell 182, 463-480.e430 (2020).
13. Kurt, I. C. et al. CRISPR C-to-G base editors for inducing targeted DNA transversions in human cells. Nature Biotechnology 39, 41-46 (2020).
14. Zhao, D. et al. Glycosylase base editors enable C-to-A and C-to-G base changes. Nature Biotechnology 39, 35-40 (2020).
15. Chen, L. et al. Programmable C:G to G:C genome editing with CRISPR-Cas9-directed base excision repair proteins. Nature Communications 12 (2021).
16. Liu, D. R. & Koblan, L. W. Cytosine to Guanine Base Editor. World Intellectual Property Organization (2018).
17. Marquart, K. F. et al. Predicting base editing outcomes with an attention-based deep learning algorithm trained on high-throughput target library screens. bioRxiv (2020).
18. Sang, P. B., Srinath, T., Patil, A. G., Woo, E.-J. & Varshney, U. A unique uracil-DNA binding protein of the uracil DNA glycosylase superfamily. Nucleic Acids Res 43, 8452-8463 (2015).
19. Ahn, W.-C. et al. Covalent binding of uracil DNA glycosylase UdgX to abasic DNA upon uracil excision. Nat Chem Biol 15, 607-614 (2019).
20. Tu, J., Chen, R., Yang, Y., Cao, W. & Xie, W. Suicide inactivation of the uracil DNA glycosylase UdgX by covalent complex formation. Nat Chem Biol 15, 615-622 (2019).
21. Gilbert, L. A. et al. CRISPR-mediated modular RNA-guided regulation of transcription in eukaryotes. Cell 154, 442-451 (2013).
22. Gallina, I., Hendriks, I. A., Hoffmann, S., Larsen, N. B., Johansen, J., Colding-Christensen, C. S., Schubert, L., Sellés-Baiget, S., Fábián, Z., Kühbacher, U., Gao, A. O., Räschle, M., Rasmussen, S., Nielsen, M. L., Mailand, N., Duxin, J. P. The ubiquitin ligase RFWD3 is required for translesion DNA synthesis. Molecular Cell 81, 1-17 (2020).
23. Levy, J. M. et al. Cytosine and adenine base editing of the brain, liver, retina, heart and skeletal muscle of mice via adeno-associated viruses. Nat Biomed Eng 4, 97-110 (2020).
24. Kim, Y. B. et al. Increasing the genome-targeting scope and precision of base editing with engineered Cas9-cytidine deaminase fusions. Nature Biotechnology 35, 371-376 (2017).
25. Kleinstiver, B. P. et al. High-fidelity CRISPR-Cas9 nucleases with no detectable genome-wide off-target effects. Nature 529, 490-495 (2016).
26. Slaymaker, I. M. et al. Rationally engineered Cas9 nucleases with improved specificity. Science 351, 84-88 (2015).
27. Chen, J. S. et al. Enhanced proofreading governs CRISPR-Cas9 targeting accuracy. Nature 550, 407-410 (2017).
28. Lee, J. K. et al. Directed evolution of CRISPR-Cas9 to increase its specificity. Nature Communications 9, 3048 (2018).
29. Koblan, L. W. et al. Improving cytidine and adenine base editors by expression optimization and ancestral reconstruction. Nature Biotechnology 36, 843-846 (2018).
30. Shen, M. W. et al. Predictable and precise template-free CRISPR editing of pathogenic variants. Nature 563, 646-651 (2018).
31. Nishimasu, H. et al. Engineered CRISPR-Cas9 nuclease with expanded targeting space. Science 361, 1259-1262 (2018).
32. Stenson, P. D. et al. Human Gene Mutation Database: towards a comprehensive central mutation database. Journal of Medical Genetics 45, 124-126 (2007).
33. Frank, M. et al. The type of variants at the COL3A1 gene associates with the phenotype and severity of vascular Ehlers-Danlos syndrome. European Journal of Human Genetics 23, 1657-1664 (2015).
34. Petrucelli, N., Daly, M. B. & Feldman, G. L. Hereditary breast and ovarian cancer due to mutations in BRCA1 and BRCA2. Genetics in Medicine 12, 245-259 (2010).
35. Douglas, J. et al. NSD1 mutations are the major cause of Sotos syndrome and occur in some cases of Weaver syndrome but are rare in other overgrowth phenotypes. American journal of human genetics 72, 132-143 (2003).
36. Luna-Pelaez, N. et al. The Cornelia de Lange Syndrome-associated factor NIPBL interacts with BRD4 ET domain for transcription control of a common set of genes.
Cell Death Dis 10 (2019).
37. Anzalone, A. V. et al. Search-and-replace genome editing without double-strand breaks or donor DNA. Nature 576, 149-157 (2019).
38. Sancar, A. DNA Excision Repair. Annual Review of Biochemistry 65, 43-81 (1996).
39. Wood, R. D. DNA Repair in Eukaryotes. Annual Review of Biochemistry 65, 135-167 (1996).
40. Modrich, P. & Lahue, R. Mismatch Repair in Replication Fidelity, Genetic Recombination, and Cancer Biology. Annual Review of Biochemistry 65, 101-133 (1996).
41. Choi, J.-Y., Lim, S., Kim, E.-J., Jo, A. & Guengerich, F. P. Translesion Synthesis across Abasic Lesions by Human B-Family and Y-Family DNA Polymerases a, 6, f, 1, x, and REV1. Journal of Molecular Biology 404, 34-44 (2010).
42. Lin, W. et al. The human REV1 gene codes for a DNA template-dependent dCMP transferase. Nucleic Acids Res 27, 4468-4475 (1999).
43. Prindle, M. J. & and molecular, L.-L. A. DNA polymerase delta in DNA replication and genome maintenance. Environmental and molecular mutagenesis 53, 666-682 (2012).
44. Rees, H. A. et al. Improving the DNA specificity and applicability of base editing through protein engineering and protein delivery. Nature Communications 8, 15790 (2017).
45. Clement, K. et al. CRISPResso2 provides accurate and rapid genome editing sequence analysis. Nature Biotechnology 37, 224-226 (2019).
46. Horlbeck, M. A. et al. Compact and highly active next-generation libraries for CRISPR-mediated gene repression and activation. eLife 5 (2016).
47. Gilbert, Luke A. et al. Genome-Scale CRISPR-Mediated Control of Gene Repression and Activation. Cell 159, 647-661 (2014).
48. Sherwood, R. I. et al. Discovery of directional and nondirectional pioneer transcription factors by modeling DNase profile magnitude and shape. Nature Biotechnology 32, 171-178 (2014).
49. Paszke, A., Gross, S., Massa, F. & in neural . . . , L.-A. Pytorch: An imperative style, high-performance deep learning library. Advances in neural . . . (2019).
50. Jinek, M. et al. RNA-programmed genome editing in human cells. eLife 2, e00471 (2013).
51. Jinek, M. et al. A Programmable Dual-RNA-Guided DNA Endonuclease in Adaptive Bacterial Immunity. Science 337, 816-821 (2012).
52. Gasiunas, G., Barrangou, R., Horvath, P. & Siksnys, V. Cas9-crRNA ribonucleoprotein complex mediates specific DNA cleavage for adaptive immunity in bacteria. PNAS 109, E2579-E2586 (2012).
53. Mali, P. et al. RNA-Guided Human Genome Engineering via Cas9. Science 339, 823-826 (2013).
54. Cong, L. et al. Multiplex Genome Engineering Using CRISPR/Cas Systems. Science 339, 819-823 (2013).
55. Camps, M., Naukkarinen, J., Johnson, B. P. & Loeb, L. A. Targeted gene evolution in Escherichia coli using a highly error-prone DNA polymerase I. PNAS 100, 9727-9732 (2003).

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

What is claimed is:

1. A fusion protein comprising (i) a nucleic acid programmable DNA binding protein (napDNAbp) domain, (ii) a cytidine deaminase domain, (iii) a first uracil binding protein (UBP) domain, and (iv) a DNA repair protein.

2. The fusion protein of claim 1, wherein the DNA repair protein is selected from a DNA polymerase, an exonuclease, an RNA binding motif protein, an E3 ligase, and a translesion polymerase.

3. The fusion protein of claim 2, wherein the DNA polymerase is selected from DNA polymerase D1 (POLD1), DNA polymerase D2 (POLD2), and DNA polymerase D3 (POLD3).

4. The fusion protein of claim 2, wherein the RNA binding motif protein is X-linked (RBMX).

5. The fusion protein of claim 2, wherein the exonuclease is EX01.

6. The fusion protein of claim 2, wherein the E3 ligase is RAD18 or RFWD3.

7. The fusion protein of claim 1 or 2, wherein the DNA repair protein is encoded by a gene selected from DDX1, EXO1, POLD1, POLD2, POLD3, RAD18, RBMX, REV1, RFWD3, TIMELESS, PCNA, POLH, POLK, UBE2I, and UBE2T.

8. The fusion protein of any one of claims 1-4 and 7, wherein the DNA repair protein is selected from POLD2, RBMX, and EXO1.

9. The fusion protein of any one of claims 1-8, wherein the first UBP domain is a UNG orthologue from Mycobacterium smegmatis (UdgX) protein, or a variant thereof.

10. The fusion protein of any one of claims 1-9, wherein the first UBP domain is a UdgX protein comprising an amino acid sequence that is at least 80%, 85%, 90%, 95%, 98%, or 99% identical to the amino acid sequence of SEQ ID NO: 49 (UdgX).

11. The fusion protein of any one of claims 1-10, wherein the first UBP domain comprises the amino acid sequence of SEQ ID NO: 49.

12. The fusion protein of any one of claims 1-10, wherein the first UBP domain comprises the amino acid sequence of SEQ ID NO: 50 (UdgX*).

13. A fusion protein comprising (i) a nucleic acid programmable DNA binding protein (napDNAbp) domain, (ii) a cytidine deaminase domain, (iii) a first UBP domain, and (iv) a second UBP domain.

14. The fusion protein of claim 13 further comprising a third UBP domain.

15. The fusion protein of claim 13 or 14, wherein the first and second UBP domains each comprise a UdgX protein, or a variant thereof.

16. The fusion protein of claim 14, wherein the third UBP domain comprises a UdgX protein, or a variant thereof.

17. The fusion protein of claim 15 or 16, wherein any of the UdgX proteins comprises an amino acid sequence that is at least 80%, 85%, 90%, 95%, 98%, or 99% identical to the amino acid sequence of SEQ ID NO: 49.

18. The fusion protein of any one of claims 15-17, wherein any of the UdgX proteins comprises the amino acid sequence of SEQ ID NO: 49.

19. The fusion protein of any one of claims 1-18, wherein the cytidine deaminase domain is a deaminase from the apolipoprotein B mRNA-editing complex (APOBEC) family.

20. The fusion protein of any one of claims 1-19, wherein the cytidine deaminase domain comprises an amino acid sequence that is at least 85% identical to an amino acid sequence of any one of SEQ ID NOs: 67-101 and 695-702.

21. The fusion protein of any one of claims 1-20, wherein the cytidine deaminase domain comprises an amino acid sequence of any one of SEQ ID NOs: 67-101 and 695-702.

22. The fusion protein of any one of claims 1-21, wherein the cytidine deaminase domain is a rat APOBEC1 (rAPOBEC1) deaminase.

23. The fusion protein of any one of claims 1-22, wherein the cytidine deaminase domain is a rat APOBEC1 (rAPOBEC1) deaminase comprising one or more mutations selected from the group consisting of W90Y, R126E, and R132E of SEQ ID NO: 93, or one or more corresponding mutations in another APOBEC deaminase.

24. The fusion protein of any one of claims 1-23, wherein the cytidine deaminase domain is selected from EE (SEQ ID NO: 696), YE1 (SEQ ID NO: 697), YE2 (SEQ ID NO: 698), and YEE (SEQ ID NO: 699).

25. The fusion protein of any one of claims 1-22, wherein the cytidine deaminase is an ancestral 689 (Anc689) deaminase.

26. The fusion protein of any one of claims 1-21, wherein the cytidine deaminase domain is a rat APOBEC3A (e3A) deaminase.

27. The fusion protein of any one of claims 1-21 and 26, wherein the cytidine deaminase domain is a rat APOBEC3A (e3A) deaminase comprising a T31A mutation in SEQ ID NO: 93, or one or more corresponding mutations in another APOBEC deaminase.

28. The fusion protein of any one of claims 1-27, wherein the napDNAbp domain comprises a Cas9 domain.

29. The fusion protein of claim 28, wherein the Cas9 domain comprises an amino acid sequence that is at least 85%, 90%, 92.5%, 95%, 97%, 98%, or 99% identical to any one of SEQ ID NOs: 4-26, 726-736.

30. The fusion protein of claim 28 or 29, wherein the Cas9 domain comprises the amino acid sequence of any one of SEQ ID NOs: 4-26, 724-736.

31. The fusion protein of any one of claims 28-30, wherein the Cas9 domain is a Cas9 nickase (nCas9).

32. The fusion protein of claim 31, wherein the Cas9 nickase (nCas9) comprises an amino acid sequence that is at least 85% identical to any one of SEQ ID NOs: 10, 13, 16, 20, 21, 725, 739, 731, 732, 736, 735, and 728.

33. The fusion protein of claim 31 or 32, wherein the nCas9 comprises the amino acid sequence of any one of SEQ ID NOs: 10, 13, 16, 20, 21, 725, 739, 731, 732, 736, 735, and 728.

34. The fusion protein of any one of claims 28-33, wherein the Cas9 domain is selected from an nCas9-NG, a HypaCas9, a Hypa-nCas9, an HF-nCas9-NG, a Sniper-nCas9, an HF-Hypa-nCas9, an e-Cas9, an e-HF-Hypa-nCas9, and an e-Hypa-Cas9.

35. The fusion protein of any one of claims 28-34, wherein the Cas9 domain is an nCas9-NG, a high fidelity Cas9-NG (HF-nCas9-NG), or a Hypa-nCas9.

36. The fusion protein of any one of claims 28-30, wherein the Cas9 domain is a nuclease inactive Cas9 (dCas9).

37. The fusion protein of any one of claims 1-36, wherein the fusion protein comprises the structure [cytidine deaminase domain]-[first UBP domain]-[napDNAbp domain], wherein each instance of “]-[” comprises an optional linker.

38. The fusion protein of any one of claims 1-37, wherein the cytidine deaminase and the first UBP domain, and/or the first UBP domain and the napDNAbp domain, are fused via a linker.

39. The fusion protein of claim 38, wherein the cytidine deaminase and the first UBP domain, and/or the first UBP domain and the napDNAbp domain, are fused via a linker comprising the amino acid sequence of any one of SEQ ID NOs: 102-109 and 441.

40. The fusion protein of any one of claims 1-39, wherein the fusion protein comprises the structure [cytidine deaminase domain]-[UdgX protein]-[Cas9 nickase], wherein each instance of “]-[” comprises an optional linker.

41. The fusion protein of any one of claims 1-12 and 19-40 further comprising a second DNA repair protein.

42. The fusion protein of claim 41, wherein the second DNA repair protein is selected from POLD2, RBMX, and EXO1.

43. The fusion protein of claim 41 or 42, wherein the first DNA repair protein is a POLD2 and the second DNA repair protein is an RBMX.

44. The fusion protein of any one of claims 1-43, wherein the fusion protein comprises the structure:
NH₂-[second UBP domain]-[cytidine deaminase domain]-[first UBP domain]-[napDNAbp domain]-COOH;
NH₂-[second UBP domain]-[cytidine deaminase domain]-[first UBP domain]-[napDNAbp domain]-[DNA repair protein]-COOH;
NH₂-[DNA repair protein]-[cytidine deaminase domain]-[first UBP domain]-[napDNAbp domain]-COOH;
NH₂-[second UBP domain]-[cytidine deaminase domain]-[first UBP domain]-[napDNAbp domain]-[third UBP domain]-COOH;
NH₂-[DNA repair protein]-[cytidine deaminase domain]-[first UBP domain]-[napDNAbp domain]-[second UBP domain]-COOH; and
NH₂-[DNA repair protein]-[cytidine deaminase domain]-[first UBP domain]-[napDNAbp domain]-[second DNA repair protein]-COOH;
wherein each instance of “]-[” comprises an optional linker.

45. The fusion protein of claim 44, wherein the second UBP domain and the cytidine deaminase domain are fused via a linker comprising the amino acid sequence of any one of SEQ ID NOs: 102-109 and 441.

46. The fusion protein of claim 44, wherein the DNA repair protein and the cytidine deaminase domain are fused via a linker comprising the amino acid sequence of any one of SEQ ID NOs: 102-109 and 441.

47. The fusion protein of claim 44, wherein the napDNAbp domain and the DNA repair protein are fused via a linker comprising the amino acid sequence of any one of SEQ ID NOs: 102-109 and 441.

48. The fusion protein of claim 44, wherein the napDNAbp domain and the second DNA repair protein are fused via a linker comprising the amino acid sequence of any one of SEQ ID NOs: 102-109 and 441.

49. The method of any one of claims 38, 39, and 45-48, wherein the linker is 32 amino acids in length.

50. The fusion protein of any one of claims 1-49 further comprising one or more nuclear localization sequences (NLS).

51. The fusion protein of claim 50, wherein the one or more NLSs is a bipartite NLS (BPNLS).

52. The fusion protein of claim 50 or 51, wherein the one or more nuclear localization sequences comprises an amino acid sequence selected from PKKKRKV (SEQ ID NO: 41), MDSLLMNRRKFLYQFKNVRWAKGRRETYLC (SEQ ID NO: 42), KRTADGSEFESPKKKRKV (SEQ ID NO: 43), and KRTADGSEFEPKKKRKV (SEQ ID NO: 440).

53. The fusion protein of any one of claims 50-52, wherein the one or more nuclear localization sequences comprises the amino acid sequence KRTADGSEFESPKKKRKV (SEQ ID NO: 43) or KRTADGSEFEPKKKRKV (SEQ ID NO: 440).

54. The fusion protein of any one of claims 51-53, wherein the fusion protein comprises the structure:
NH₂-[BPNLS]-[second UBP domain]-[cytidine deaminase domain]-[first UBP domain]-[napDNAbp domain]-[BPNLS]-COOH;
NH₂-[BPNLS]-[second UBP domain]-[cytidine deaminase domain]-[first UBP domain]-[napDNAbp domain]-[DNA repair protein]-[BPNLS]-COOH;
NH₂-[BPNLS]-[DNA repair protein]-[cytidine deaminase domain]-[first UBP domain]-[napDNAbp domain]-[BPNLS]-COOH;
NH₂-[BPNLS]-[second UBP domain]-[cytidine deaminase domain]-[first UBP domain]-[napDNAbp domain]-[third UBP domain]-[BPNLS]-COOH;
NH₂-[BPNLS]-[DNA repair protein]-[cytidine deaminase domain]-[first UBP domain]-[napDNAbp domain]-[second UBP domain]-[BPNLS]-COOH; and
NH₂-[BPNLS]-[DNA repair protein]-[cytidine deaminase domain]-[first UBP domain]-[napDNAbp domain]-[second DNA repair protein]-[BPNLS]-COOH;
wherein each instance of “]-[” comprises an optional linker.

55. The fusion protein of any one of claims 37-54, wherein the fusion protein comprises the structure:
[UdgX]-[Anc689 deaminase]-[UdgX]-[nCas9 domain];
[UdgX]-[Anc689 deaminase]-[UdgX]-[nCas9 domain]-[RBMX];
[UdgX]-[EE deaminase]-[UdgX]-[nCas9 domain]-[UdgX];
[UdgX]-[rAPOBEC1 deaminase]-[UdgX]-[HF-nCas9 domain];
[UdgX]-[rAPOBEC1 deaminase]-[UdgX]-[HF-nCas9 domain]-[UdgX];
[RBMX]-[e3A deaminase]-[UdgX]-[nCas9 domain];
[RBMX]-[e3A deaminase]-[UdgX]-[HF-nCas9 domain];
[POLD2]-[rAPOBEC1 deaminase]-[UdgX]-[nCas9 domain];
[POLD2]-[rAPOBEC1 deaminase]-[UdgX]-[nCas9 domain]-[UdgX];
[POLD2]-[rAPOBEC1 deaminase]-[UdgX]-[nCas9 domain]-[RBMX];
[EXO1]-[rAPOBEC1 deaminase]-[UdgX]-[nCas9 domain];
[UdgX]-[Anc689 deaminase]-[UdgX]-[nCas9-NG domain]-[RBMX];
[UdgX]-[rAPOBEC1 deaminase]-[UdgX]-[nCas9-NG domain]; and
[UdgX]-[rAPOBEC1 deaminase]-[UdgX]-[HF-nCas9-NG domain],
wherein each instance of “]-[” comprises an optional linker.

56. The fusion protein of any one of claims 37-55, wherein the fusion protein comprises the structure: [POLD2]-[rAPOBEC1 deaminase]-[UdgX]-[nCas9 domain]-[UdgX]; [UdgX]-[EE deaminase]-[UdgX]-[nCas9 domain]-[UdgX]; or [UdgX]-[Anc689 deaminase]-[UdgX]-[nCas9 domain]-[RBMX].

57. A complex comprising a guide RNA molecule and the fusion protein of any one of claims 1-56.

58. The complex of claim 57, wherein the guide RNA is from 15-100 nucleotides long and comprises a sequence of at least 10, at least 15, or at least 20 contiguous nucleotides that is complementary to a target sequence.

59. The complex of claim 57 or 58, wherein the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

60. The complex of any one of claims 57-59, wherein the guide RNA is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, or 200 nucleotides long.

61. The complex of any one of claims 57-60, wherein the target sequence is a DNA sequence.

62. The complex of any one of claims 57-61, wherein the target sequence is in the genome of an organism.

63. The complex of claim 62, wherein the organism is a bacteria.

64. The complex of claim 62, wherein the organism is a eukaryote.

65. The complex of claim 62, wherein the organism is a plant or fungus.

66. The complex of claim 62, wherein the organism is a vertebrate.

67. The complex of claim 66, wherein the vertebrate is a mammal.

68. The complex of claim 67, wherein the mammal is a rodent.

69. The complex of claim 67, wherein the mammal is a human.

70. The complex of any one of claims 57-69, wherein the target sequence is in the genome of a cell.

71. The complex of claim 70, wherein the cell is a mouse cell, a rat cell, or human cell.

72. A polynucleotide encoding the fusion protein of any one of claims 1-56.

73. A vector comprising the polynucleotide of claim 72.

74. The vector of claim 73, wherein the vector comprises a heterologous promoter driving expression of the polynucleotide.

75. The vector of 73 or 74 further comprising a polynucleotide encoding a gRNA.

76. The vector of any one of claims 73-76, wherein the vector is a recombinant AAV vector.

77. The vector of claim 76, wherein the recombinant AAV vector is a dual recombinant AAV vector.

78. A cell comprising the fusion protein of any one of claims 1-56 or the complex of any one of claims 57-71.

79. A cell comprising the polynyucleotide of claim 576 or the vector of any one of claims 577-B21.

80. A pharmaceutical composition comprising the fusion protein of any one of claims 1-56, the complex of any one of claims 57-71, the vector of any one of claims 73-77, or the cell of claim 78 or 79.

81. The pharmaceutical composition of claim 80 further comprising a pharmaceutically acceptable excipient.

82. A method comprising contacting a nucleic acid molecule with the fusion protein of any one of claims 1-56 or the complex of any one of claims 57-71.

83. The method of claim 82, wherein the nucleic acid comprises a target sequence in the genome of a cell.

84. The method of claim 82 or 83, wherein the nucleic acid is DNA.

85. The method of claim 83 or 84, wherein the target sequence comprises a sequence associated with a disease or disorder.

86. The method of any one of claims 83-85, wherein the target sequence comprises a sequence in a gene selected from COL31, BR83, NSD1, and NIPBL.

87. The method of any one of claims 83-86, wherein the target sequence comprises a point mutation associated with a disease or disorder.

88. The method of claim 87, wherein the activity of the fusion protein or the complex results in a correction of the point mutation.

89. The method of any one of claims 83-88, wherein the target sequence comprises a G to C point mutation associated with a disease or disorder, and wherein a deamination of the mutant C base and excision of the resulting uracil results in a sequence that is not associated with the disease or disorder.

90. The method of any one of claims 83-88, wherein the target sequence comprises a C to G point mutation associated with a disease or disorder, and wherein a deamination of the C base that is complementary to the G base of the C to G point mutation, and excision of the resulting uracil, results in a sequence that is not associated with the disease or disorder.

91. A method comprising contacting a nucleic acid molecule that comprises a target sequence with a guide RNA and a fusion protein selected from Anc689-nCas9, e3A-nCas9, EE-nCas9, and rAPOBEC1-nCas9;
wherein the target sequence comprises a G to C point mutation associated with a disease or disorder, and wherein a deamination of the mutant C base, and excision of the resulting uracil, results in a sequence that is not associated with the disease or disorder.

92. A method comprising contacting a nucleic acid molecule that comprises a target sequence with a guide RNA and a fusion protein selected from Anc689-nCas9, e3A-nCas9, EE-nCas9, and rAPOBEC1-nCas9;
wherein the target sequence comprises a C to G point mutation associated with a disease or disorder, and wherein a deamination of the C base that is complementary to the G base of the C to G point mutation, and excision of the resulting uracil, results in a sequence that is not associated with the disease or disorder.

93. The method of any one of claims 83-92, wherein the target sequence encodes a protein, and wherein the point mutation is in a codon and results in a change in the amino acid encoded by the mutant codon as compared to a wild-type codon.

94. The method of claim 93, wherein the deamination and excision results in a change of the amino acid encoded by the mutant codon.

95. The method of claim 93 or 94, wherein the deamination and excision generates the codon encoding a wild-type amino acid.

96. The method of any one of claims 82-95, wherein the step of contacting is performed in vivo in a subject.

97. The method of any one of claims 82-95, wherein the step of contacting is performed in vitro or ex vivo.

98. The method of claim 96, wherein the subject has been diagnosed with a disease or disorder.

99. The method of claim 98, wherein the disease or disorder is Ehlers-Danlos syndrome, Sotos syndrome, Cornelia de Lange syndrome, or a cancer.

100. The method of any one of claims 82-99, wherein the target sequence comprises the DNA sequence RCTA or TCR, wherein R may be any nucleotide.

101. The method of claim 100, wherein the target sequence comprises the DNA sequence ACTA.

102. The method of any one of claims 82-101, wherein the product purity of conversion of the C to a G is at least 65%, 70%, 73%, 75%, 77%, 80%, 82%, 83%, 84%, 86%, 88%, 90%, 92.5%, or 95%.

103. The method of claim 102, wherein the product purity is at least 83%.

104. The method of claim 102, wherein the product purity is at least 73%.

105. The method of any one of claims 82-104, wherein the average efficiency of conversion of the C to a G is at least 70%, 73%, 75%, 77%, 80%, 82%, 83%, 84%, 86%, 88%, 90%, 92.5%, 95%, or 98%.

106. The method of any one of claims 82-105, wherein the average off-target editing frequency is less than 6%, less than 5%, less than 4%, less than 3%, less than 2%, less than 1.25%, less than 1%, less than 0.75%, less than 0.5%, less than 0.4%, less than 0.25%, less than 0.2%, less than 0.15%, or less than 0.1%.

107. A method for editing a nucleobase pair of a double-stranded DNA sequence, the method comprising:
contacting a target region of the double-stranded DNA sequence with a complex comprising the fusion protein of any one of claims 1-56, or any of the fusion proteins in accordance with claim 91 or 92, and a guide nucleic acid, wherein the target region comprises a target nucleobase pair; and thereby:
inducing strand separation of the target region;
converting a cytosine of the target nucleobase pair in a single strand of the target region to a uracil;
excising the uracil from the double-stranded DNA sequence to produce an abasic site, wherein a guanine opposite the abasic site is replaced by a cytosine;
cutting no more than one strand of the target region; and
inserting a guanine into the abasic site, and thereby generating an intended edited base pair.

108. The method of claim 107, wherein the method causes less than 20% to less than 1% indel formation.

109. The method of claim 107 or 108, wherein the efficiency of generating the intended edited base pair is at least 73%.

110. The method of claim 107 or 108, wherein the ratio of intended edited basepairs to unintended edited basepairs is between 2:1 and 10:1.

111. The method of claim 107 or 108, wherein the ratio of intended edited basepairs to indel formation is between 2:1 and 200:1.

112. The method of any one of claims 107-111, wherein the intended edited base pair is upstream of a PAM site.

113. The method of any one of claims 107-111, wherein the intended edited base pair is downstream of a PAM site.

114. The method of any one of claims 107-113, wherein the target region comprises a target window, wherein the target window comprises the target nucleobase pair.

115. The method of claim 114, wherein the target window comprises 3-8 nucleotides.

116. The method of claim 114 or 115, wherein the target window is 1-8, 1-7, 1-6, 1-5, 1-4, or 1-3 nucleotides in length.

117. The method of any one of claims 114-116, wherein the target window is 1, 2, 3, 4, 5, 6, 7, 8, 9, 10, or more than 10 nucleotides in length.

118. The method of any one of claims 114-117, wherein the target window comprises the intended edited base pair.

119. A method of using a machine learning model to identify at least one fusion protein from among a set of one or more fusion proteins, for use in a base editing system for introducing a cytosine to guanine change in a nucleotide sequence, the method comprising:
using software executing on at least one computer hardware processor to perform:
obtaining input data indicative of the nucleotide sequence, one or more guide RNAs, and the set of fusion proteins, wherein the at least one fusion protein comprises a napDNAbp domain, a cytidine deaminase domain, and at least one uracil binding protein;
generating first input features from the input data;
applying a first machine learning model to the first input features to obtain first output data indicative, for each fusion protein in the set, of a base editing efficiency at one or multiple locations in the nucleotide sequence, of the base editing system when using the each fusion protein;
generating second input features from the input data;
applying a second machine learning model to the second input features to obtain second output data indicative, for each fusion protein in the set, of a base editing product purity at one or multiple locations in the nucleotide sequence, by the base editing system when using the each fusion protein; and
identifying, using the first output data and the second output data, the at least one fusion protein for use in the base editing system for introducing the cytosine to guanine change in the nucleotide sequence.

120. The method of claim 119 further comprising applying a third machine learning model to the second input features to obtain third output data indicative, for each fusion protein in the set, of a bystander editing efficiency at one or multiple locations in the nucleotide sequence, by the base editing system when using the each fusion protein.

121. The method of claim 119 or 120, wherein the set of fusion proteins comprises the fusion protein of any one of claims 1-56 and any of the fusion proteins in accordance with claim 820 or 821.

122. A method of treating a subject having or suspected of having a disease or disorder comprising administering the fusion protein of any one of claims 1-56, the complex of any one of claims 57-71, the polynucleotide of claim 72, the vector of any one of claims 73-77, the cell of claim 78 or 79, or the pharmaceutical composition of claim 80 or 81 to the subject.

123. The method of claim 122, wherein the subject is a human.

124. The method of claim 122 or 123, wherein the disease or disorder is Ehlers-Danlos syndrome, Sotos syndrome, Cornelia de Lange syndrome, or a cancer.

125. Use of (a) the fusion protein of any one of claims 1-56 and (b) a guide RNA targeting the fusion protein of (a) to a target C:G nucleobase pair in a double-stranded DNA molecule in DNA editing.

126. The use of claim 125, whereby the DNA editing comprises nicking one strand of the double-stranded DNA, wherein the one strand comprises the G of the target C:G nucleobase pair.

127. Use of the fusion protein of any one of claims 1-56, the complex of any one of claims 57-71, the vector of any one of claims 73-77, the cell of claim 78 or 79, or the pharmaceutical composition of claim 80 or 81 as a medicament.

128. A kit comprising a nucleic acid construct, comprising
(a) a nucleic acid sequence encoding the fusion protein of any one of claims 1-56;
(b) a nucleic acid sequence encoding a gRNA; and
(c) one or more heterologous promoters that drive the expression of the sequence of (a) and/or the sequence of (b).
